(19) United States
(12) Patent Application Publication
(10) Pub. No.: US 2023/0183753 A1
(43) Pub. Date: Jun. 15, 2023

(54) Methods of in Vitro Cell Delivery

(71) Applicant: Intellia Therapeutics, Inc., Cambridge, MA (US)

(72) Inventors: Pooja Kyatsandra Narendra, Cambridge, MA (US); Sean Michael Burns, Hingham, MA (US); Paula Gutierrez Martinez, Cambridge, MA (US); Arti Mahendra Prakash Kanjolia, Malden, MA (US); Anthony Monti, Cambridge, MA (US); Aaron Prodeus, Cambridge, MA (US); Mohamed Simo Arredouani, Belmont, MA (US); Özgün Kiliç, Cambridge, MA (US); Reed Walker LaRiviere, Cambridge, MA (US); Palak Sushil Sharma, Cambridge, MA (US); Eleni Stampouloglou, Cambridge, MA (US); Qingzhan Zhang, Cambridge, MA (US)

(73) Assignee: Intellia Therapeutics, Inc., Cambridge, MA (US)

(21) Appl. No.: 18/050,333

(22) Filed: Oct. 27, 2022

Related U.S. Application Data
(63) Continuation of application No. PCT/US2021/029446, filed on Apr. 27, 2021.
(60) Provisional application No. 63/176,221, filed on Apr. 17, 2021.
(60) Provisional application No. 63/165,619, filed on Mar. 24, 2021.
(60) Provisional application No. 63/130,100, filed on Dec. 23, 2020.
(60) Provisional application No. 63/124,058, filed on Dec. 11, 2020.
(60) Provisional application No. 63/121,781, filed on Dec. 4, 2020.
(60) Provisional application No. 63/016,913, filed on Apr. 28, 2020.

(51) Int. Cl. C12N 15/90; C12N 5/0783; C12N 15/87; C12N 15/11; C12N 9/22; A61K 38/17; A61K 35/17; A61P 35/00

(52) U.S. Cl. CPC C12N 15/907; C12N 5/0636; C12N 15/87; C12N 15/11; C12N 9/22; A61K 38/1774; A61K 35/17; A61P 35/00; C12N 2510/00; C12N 2310/20; C12N 2800/80

(57) ABSTRACT

Compositions and methods for multiplex delivery and gene editing in vitro are provided.

This patent application is a continuation application of International Application No. PCT/US2021/029446, filed on Apr. 27, 2021, which claims the benefit of priority to U.S. Provisional Application No. 63/176,221, filed Apr. 17, 2021, U.S. Provisional Application No. 63/165,619, filed Mar. 24, 2021, U.S. Provisional Application No. 63/130,100, filed Dec. 23, 2020, U.S. Provisional Application No. 63/124,058, filed Dec. 11, 2020, U.S. Provisional Application No. 63/121,781, filed Dec. 4, 2020, and U.S. Provisional Application No. 63/016,913, filed Apr. 28, 2020, the contents of each of which are incorporated herein by reference in their entirety for all purposes.

The patent application is filed with a sequence listing in electronic format. The Sequence Listing is provided as a file entitled “2022-10-24_01155-0035-00US,” which was created on Oct. 24, 2022, and which is 288,914 bytes in size. The information in the electronic format of the sequence listing is incorporated herein by reference in its entirety

The ability to introduce multiple genetic edits into a cell in vitro is of interest for gene editing and clinical therapeutic applications. For example, adoptive cell therapy approaches using genetically modified immune cells have become an attractive modality to treat a variety of conditions and diseases, including cancers, to reconstitute cell lineages and immune system defense. However, the clinical application of cell product therapies has been challenging in part due to the complex genetic engineering requirements. The ability to engineer multiple attributes into a single cell depends on the ability to efficiently perform edits in multiple targeted genes, including knockouts and in locus insertions, while retaining viability and the desired cell phenotype.

CRISPR/Cas9 genome editing has been demonstrated to be highly efficient, however, simultaneous edits in different loci have been reported to result in poorer cell survival, increased translocations, which potentially impair the quality and safety of the cell product, and decreased gene editing efficiencies as the number of edits increase. Existing cell engineering technologies, including electroporation, present limitations in providing the necessary cell quality and yield using a sequential editing process due to the cumulative toxicity to the cell. Moreover, certain cell types, including for example, T cells, have proven particularly difficult for permanent multiplex editing in vitro.

Thus, there is a need for safer processes for delivering multiple genome editing tools to a cell and for performing gene editing.

The methods provided herein comprise using lipid nucleic acid assembly compositions (e.g., lipid nanoparticles (“LNPs”)) for safer delivery of genome editing tools and for multiplex genome editing applications providing substantial advantages over traditional methods.

In some embodiments, the methods produce cells with a lower toxicity profile, fewer translocations, and greater survival and expansion, thereby shortening the time required for manufacturing and increasing yield. In some embodiments, the methods provide for highly efficient multiplex editing in T cells in vitro to replace the endogenous T cell receptor (TCR) with a therapeutic TCR, resulting in engineered T cells with increased cytokine production, favorable early-stem cell memory phenotype, and continued proliferation with antigen-specific stimulation.

### BRIEF DESCRIPTION OF THE DRAWINGS

FIG. 1 shows the fold expansion of T cells treated with electroporation (EP) or lipid nanoparticles (LNPs), with and without AAV, after 10 days in culture post-editing.

FIG. 2 shows the percentage of CD3+Vb8+ TCR T cells (gated on CD8+ and CD4+) treated with electroporation (EP) or lipid nanoparticles (LNP), with and without AAV, on day 7 post-editing.

FIG. 3 shows the percentage of residual endogenous TCR expressing (CD3+Vb8−) T cells (gated on CD8+ and CD4+) treated with electroporation (EP) or lipid nanoparticles (LNP), with and without AAV, on day 7 post-editing.

FIG. 4 shows staining for early stem-cell memory phenotype CD8+ T cells by flow cytometry (CD27+, CD45RA+) in EP-treated T cells and LNP-treated T cells.

FIG. 5 shows IL-2 secretion of WT1 TCR engineered T cells (EP-treated v. LNP-treated) in co-culture with OCI-AML2 cells pulsed with VLD peptide.

FIG. 6 shows IFNγ secretion of WT1 TCR engineered T cells (EP-treated v. LNP-treated) in co-culture with K562 HLA-A*02:01 positive cells.

FIG. 7 shows specific lysis by WT1 TCR engineered T cells (EP-treated v. LNP-treated) of K562 HLA-A*02:01 positive cells.

FIG. 8 shows proliferation after repeated stimulations (as cumulative fold change) for EP-treated v. LNP-treated WT1 TCR engineered T cells when co-cultured with OCI-AML3 target cells pulsed with VLD peptide.

FIG. 9 shows expansion of T cells post-editing by electroporation (“EP”), simultaneous LNPs (“SIM”), sequential process 1 (2.5 μg/ml LNPs) (“BF2.5”; TRBC targeted, then TRAC targeted), sequential process 2 (5 μg/ml LNPs) (“BF5”; TRBC targeted, then TRAC targeted), sequential process 3 (2.5 μg/ml LNPs) (“AF”; TRAC targeted, then TRBC targeted).

FIG. 10 shows transgenic TCR (tgTCR) insertion rates (% Vb8+, CD3+) post-editing by electroporation (“EP”), simultaneous LNPs (“SIM”), sequential process 1 (2.5 μg/ml LNPs) (“BF2.5”; TRBC targeted, then TRAC targeted), sequential process 2 (5 μg/ml LNPs) (“BF5”; TRBC targeted, then TRAC targeted), sequential process 3 (2.5 μg/ml LNPs) (“AF”; TRAC targeted, then TRBC targeted).

FIG. 11 shows the percentage of CD8+ T cells retaining endogenous TCR post-editing by electroporation (“EP”), simultaneous LNPs (“SIM”), sequential process 1 (2.5 μg/ml LNPs) (“BF2.5”; TRBC targeted, then TRAC targeted), sequential process 2 (5 μg/ml LNPs) (“BF5”; TRBC targeted, then TRAC targeted), sequential process 3 (2.5 μg/ml LNPs) (“AF”; TRAC targeted, then TRBC targeted).

FIG. 12 shows the percentage of engineered T cells that are associated with memory phenotype (CD27+) post-editing by electroporation (“EP”), simultaneous LNPs (“SIM”), sequential process 1 (2.5 μg/ml LNPs) (“BF2.5”; TRBC targeted, then TRAC targeted), sequential process 2 (5 μg/ml LNPs) (“BF5”; TRBC targeted, then TRAC targeted), sequential process 3 (2.5 μg/ml LNPs) (“AF”; TRAC targeted, then TRBC targeted).

FIGS. 13A-B show the percentage of TRAC-TRBC translocated cells and cells with TCR insertion into the TRBC loci in engineered T cells post-editing by electroporation (“EP”), simultaneous LNPs (“SIM”), sequential process 1 (2.5 μg/ml LNPs) (“BF2.5”; TRBC targeted, then TRAC targeted), sequential process 2 (5 μg/mL LNPs) (“BF5”; TRBC targeted, then TRAC targeted), sequential process 3 (2.5 μg/mL LNPs) (“AF”; TRAC targeted, then TRBC targeted); translocations detected with TRAC probe are shown in FIG. 13A and TRBC probe in FIG. 13B.

FIGS. 14A-B show the percentage of TRBC-TRAC translocated cells and cells with TCR insertion into the TRBC loci in engineered T cells post-editing by electroporation (“EP”), simultaneous LNPs (“SIM”), sequential process 1 (2.5 μg/ml LNPs) (“BF2.5”; TRBC targeted, then TRAC targeted), sequential process 2 (5 μg/mL LNPs) (“BF5”; TRBC targeted, then TRAC targeted), sequential process 3 (2.5 μg/mL LNPs) (“AF”; TRAC targeted, then TRBC targeted); translocations detected with TRAC probe are shown in FIG. 14A and TRBC probe in FIG. 14B.

FIGS. 14C-D show the percentage of TRAC-TRBC translocated cells in engineered T cells post-editing by electroporation (“EP”), simultaneous LNPs (“SIM”), sequential process 1 (2.5 μg/ml LNPs) (“BF2.5”; TRBC targeted, then TRAC targeted), sequential process 2 (5 μg/mL LNPs) (“BF5”; TRBC targeted, then TRAC targeted), sequential process 3 (2.5 μg/mL LNPs) (“AF”; TRAC targeted, then TRBC targeted); translocations detected with TRAC probe are shown in FIG. 14C and TRBC probe in FIG. 14D.

FIGS. 14E-F show the percentage of TRBC-TRAC translocated cells in engineered T cells post-editing by electroporation (“EP”), simultaneous LNPs (“SIM”), sequential process 1 (2.5 μg/ml LNPs) (“BF2.5”; TRBC targeted, then TRAC targeted), sequential process 2 (5 μg/mL LNPs) (“BF5”; TRBC targeted, then TRAC targeted), sequential process 3 (2.5 μg/mL LNPs) (“AF”; TRAC targeted, then TRBC targeted); translocations detected with TRAC probe are shown in FIG. 14E and TRBC probe in FIG. 14F.

FIGS. 15A-F shows T cell mediated cytotoxicity of WT1 TCR engineered T cells as assessed by a luciferase-based target cell killing assay. Engineered T cells were co-cultured with K562 cells (FIG. 15A and FIG. 15D), K562-A2.1 cells (FIG. 15B and FIG. 15E), 697-luc cells (FIG. 15C and FIG. 15F).

FIG. 16 shows tgTCR insertion (Vb8+, CD3+) rates for engineered T cells as assessed by flow cytometry (EP-treated v. LNP-treated).

FIG. 17 shows the percentage of CD8+ T cells with inserted GFP (CD3−, GFP+) or retaining endogenous TCR (CD3+) post-editing as assessed by flow cytometry (EP-treated v. LNP-treated).

FIG. 18 shows the percentage of engineered T cells that are associated with memory phenotype (CD27+, CD45RO−) post-editing (EP-treated v. LNP-treated).

FIG. 19 shows liquid tumor burden in NOG-hIL-2 mice following treatment with engineered T cells; bioluminescence was used as a measure of leukemic tumor burden.

FIG. 20 shows the percent survival of NOG-hIL-2 mice following treatment with engineered T cells.

FIGS. 21A and 21B show the percentage of β-2 microglobulin (B2M) negative cells (FIG. 21A) by flow cytometry and percent B2M editing by NGS (FIG. 21B) in response to LNP dose.

FIGS. 22A and 22B show the percentage of TRAC negative cells (FIG. 22A) by flow cytometry and percent TRAC indel (FIG. 22B) by NGS in response to LNP dose.

FIGS. 23A and 23B show the percentage of editing by NGS before MACS processing (FIG. 23A) and after MACS processing (FIG. 23B).

FIGS. 24A and 24B show the protein expression of engineered T cells by flow cytometry before MACS processing (FIG. 24A) and after MACS processing (FIG. 24B).

FIG. 25 shows the chromosomal structural variations in engineered cells by KromaTiD dGH assay.

FIG. 26 shows the mean editing percentage (expressed as % indels) for T cells edited using mRNA and gRNA delivery with different ionizable lipid formulations.

FIG. 27 shows the time to reach editing plateau in T cells edited using mRNA and gRNA delivery with different ionizable lipid formulations.

FIG. 28 shows the percentage of CD3− cells by flow cytometry in T cells treated with LNPs and different serum factors.

FIG. 29 shows the frequency of B2M negative T cells (treated with lipoplex) by flow cytometry.

FIG. 30 shows editing frequency (indels) of lipoplex-treated T cells.

FIG. 31 shows the effect of media composition on percent editing in activated T cells, indicating delivery of Cas9 mRNA and gRNA by LNPs.

FIG. 32 shows the effect of media composition on percent editing in non-activated T cells, indicating delivery of Cas9 mRNA and gRNA by LNPs.

FIG. 33 shows editing frequency in lymphoblastoid cells treated with LNPs delivering an RNA-guided DNA binding agent mRNA and gRNA.

FIG. 34 shows the percentage of B2M negative lymphoblastoid cells treated with LNPs delivering an RNA-guided DNA binding agent mRNA and gRNA.

FIG. 35 shows the percentage of engineered T cells with multiple insertions (TCR insertion and GFP insertion) by flow cytometry following simultaneous delivery with LNPs.

FIG. 36 shows the percentage of engineered T cells with residual TCR or residual HLA-ABC expression by flow cytometry following simultaneous delivery with LNPs.

FIG. 37 shows a heat map of transcript levels for engineered T cells.

FIGS. 38A-D show an experimental schematic and leukemic blast levels for mice treated with engineered WT1 T cells and controls. FIG. 38A shows a timeline and schematic of the in vivo experiment. FIG. 38B shows AML leukemic blasts outgrowth upon treatment of mice with engineered WT1-T cells generated with an electroporation process or with an LNP process, as compared to T cells transduced with an unrelated MART1-TCR, or another control without any treatment (leukemic blasts only). Leukemia occurrence was measured over time as cells per microliter of blood. FIG. 38C shows the percentage of AML cells per total live cells in bone marrow upon treatment of the groups of mice. FIG. 38D shows the percentage of AML cells per total live cells in spleen upon treatment of the groups of mice.

FIGS. 39A-D show the editing profiles of T cells when treated with varying levels of BC22n (“BC22n,” as used herein, refers to BC22 without UGI) mRNA and Cas9 mRNAs. Cells were edited with individual guide RNAs G015995 (FIG. 39A), G016017 (FIG. 39B), G016206 (FIG. 39C), and G018117 (FIG. 39D).

FIGS. 40A-D show the editing profiles for T cells edited with four guides simultaneously using varying levels of BC22n mRNA or Cas9 mRNAs. The editing profile at each edited locus is represented separately: G015995 (FIG. 40A), G016017 (FIG. 40B), G016206 (FIG. 40C), and G018117 (FIG. 40D).

FIGS. 41A-H show phenotyping results as percent of cells negative for antibody binding with increasing total RNA for both BC22 and Cas9 samples. FIG. 41A shows the percentage of B2M negative cells when B2M guide G015995 was used for editing. FIG. 41B shows the percentage of B2M negative cells when multi guides were used for editing. FIG. 41C shows the percentage of CD3 negative cells when TRAC guide G016017 was used for editing. FIG. 41D shows the percentage of CD3 negative cells when TRBC guide G016206 was used for editing. FIG. 41E shows the percentage of CD3 negative cells when multiple guides were used for editing. FIG. 41F shows the percentage of MHC II negative cells when CIITA guide G018117 was used for editing. FIG. 41G shows the percentage of MEW II negative cells when multiple guides were used for editing. FIG. 41H shows the percentage of triple (B2M, CD3, MEW II) negative cells when multiple guides were used for editing.

FIG. 42 shows the cell viability relative to untreated cells following electroporation or LNP delivery of BC22n or Cas9 editors and single or multiple guides.

FIG. 43 shows the total γH2AX spot intensity per nuclei following electroporation or LNP delivery of BC22n or Cas9 editors and single or multiple guides.

FIG. 44 shows the percentage editing at loci of interest following LNP delivery of BC22n or Cas9 editors and single or multiple guides.

FIG. 45 shows the percentage of negative cells for stated surface proteins following LNP delivery of BC22n or Cas9 editors and single or multiple guides.

FIG. 46 shows the percentage of interchromosomal translocations among total unique molecules following LNP delivery of BC22n or Cas9 editors and multiple guides.

FIGS. 47A-F show results for sequential editing in CD8+ T cells. FIG. 47A shows the percentage of HLA-A positive cells. FIG. 47B shows the percentage of MEW class II positive cells. FIG. 47C shows the percentage of WT1 TCR positive CD3+, Vb8+ cells. FIG. 47D shows the percentage of CD3+, V8_{low} cells displaying mis-paired TCRs. FIG. 47E shows the percentage of CD3+, vb8− cells displaying only endogenous TCRs. FIG. 47F shows the percentage of CD3+, Vb8+, positive for the WT1 TCR and negative for HLA-A and MEW class II.

FIGS. 48A-F show results for sequential editing in CD4+ T cells. FIG. 48A shows the percentage of HLA-A positive cells. FIG. 48B shows the percentage of MEW class II positive cells. FIG. 48C shows the percentage of WT1 TCR positive CD3+, Vb8+ cells. FIG. 48D shows the percentage of CD3+, V8_{low} cells displaying mis-paired TCRs. FIG. 48E shows the percentage of CD3+, vb8− cells displaying only endogenous TCRs. FIG. 48F shows the percentage of CD3+, Vb8+, positive for the WT1 TCR and negative for HLA-A and MEW class II.

FIGS. 49A-D show the percent indels following sequential editing of T cells for CIITA (FIG. 49A), HLA-A (FIG. 49B), TRBC1 (FIG. 49C), and TRBC2 (FIG. 49D) in T cells.

FIG. 50A shows the percent of CD3eta+, Vb8− cells, representing the population of T cells without gene disruption at the TRAC or TRBC1/2 loci.

FIG. 50B shows the percent of CD3eta+, Vb8+ cells, representing the population of T cells with WT1 TCR insertion at the TRAC.

FIG. 50C shows the percent of HLA-A2− cells, representing the population of T cells with effective gene disruption at the HLA locus.

FIG. 50D shows the percent of HLA-DRDPDQ− cells, representing the population of T cells with effective gene disruption at the CIITA locus.

FIG. 50E shows the percent of GFP+ cells, representing the population of T cells with GFP insertion at the AAVS1 locus.

FIG. 50F shows the percent of Vb8+ GFP+ HLA-A− HLA-DRDPDQ− cells, representing the population of T cells harboring 5 genome edits.

FIG. 51A shows the percent CD3 negative cells representing the population of T cells with effective gene disruption at the TRBC1/2 loci after activated T cells were treated with LNPs preincubated with differing levels of Apo protein.

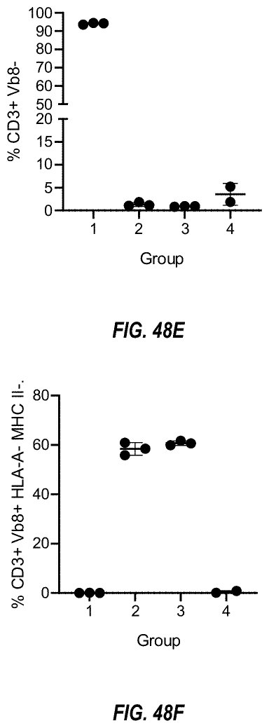

FIG. 51B shows the percent CD3 negative cells representing the population of T cells with effective gene disruption at the TRBC1/2 loci after non-activated T cells were treated with LNPs preincubated with differing levels of Apo protein.

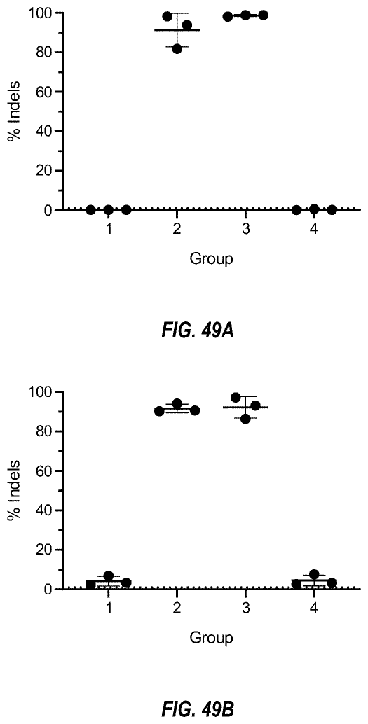

FIG. 52A shows percent CD3 negative cells representing the population of T cells with effective gene disruption at the TRAC locus after non-activated T cell treatment at 0 hours with co-formulated or mRNA-only first LNPs formulated with PEG-2kDMG and treatment with gRNA-only second LNPs at 0 hours or 72 hours.

FIG. 52B shows percent CD3 negative cells representing the population of T cells with effective gene disruption at the TRAC locus after non-activated T cell treatment at 0 hours with co-formulated or mRNA-only first LNPs formulated with PEG-Lipid H and treatment with gRNA-only second LNPs at 0 hours or 72 hours.

FIG. 53A shows the percent of CD3− cells representing the population of T cells with effective gene disruption at the TRAC locus after activated T cell treatment with LNPs formulated with varied lipid molar ratios.

FIG. 53B shows the percent of CD3− cells representing the population of T cells with effective gene disruption at the TRAC locus after non-activated T cell treatment with LNPs formulated with varied lipid molar ratios.

FIG. 54 shows the percent CD3− cells representing the population of T cells with effective gene disruption at the TRAC locus after activated T cells treatment with LNPs formulated with varied w/w ratios of mRNA and sgRNA.

FIGS. 55A-B show the percent CD3− cells representing the population of T cells with effective gene disruption at the TRAC locus after non-activated T cell treatment with LNPs formulated with varied w/w ratios of mRNA and sgRNA. FIG. 55A shows Donor 1. FIG. 55B shows Donor 2.

FIGS. 56A-B show the percentage of CD86+ cells out of CD20+ representing the population of activated B cells after culture under various media conditions. FIG. 56A shows cells cultured in IMDM based media. FIG. 56B shows cells cultured in StemSpan based media.

FIGS. 56C-D show the percentage of LDLR+ cells out of CD20+ B cells after culture under various media conditions. FIG. 56C shows cells cultured in IMDM based media. FIG. 56D shows cells cultured in StemSpan based media.

FIGS. 57A-B show the fold expansion at Day 14 of B cells cultured in media containing 1, 10 or 100 ng/ml CD40L. FIG. 57A shows cells stimulated for primary activation only. FIG. 57B shows cells stimulated for secondary activation (plasmablast differentiation).

FIGS. 58A-B show mean percent editing as determined by NGS in B cells following editing with LNPs formulated with stated lipids. FIG. 58A shows B cells cultured in IMDM.

FIG. 58B shows B cells cultured in StemSpan.

FIG. 59 shows the percent of B2M negative cells representing the population of B cells with effective gene disruption following treatment with LNPs formulated with Lipid A or Lipid D and pre-incubated with ApoE3 or ApoE4.

FIGS. 60A-B show percent B2M negative cells representing the population of B cells with effective gene disruption following treatment with LNPs formulated with Lipid A or Lipid D. FIG. 60A shows LNP treatment from 1 day before activation to 5 days after activation.

FIG. 60B shows treatment with LNP formulated with Lipid A from 6 to 10 days after activation.

FIG. 61 shows the percent of B2M negative cells representing the population of B cells with effective gene disruption following editing with DNAPK inhibitors Compound 1 or Compound 4.

FIG. 62 shows percent editing assessed by NGS in NK cells treated with LNPs formulated with stated lipids.

FIG. 63 shows percent editing assessed by NGS in NK cells treated with varying does of LNP at 14 days post LNP treatment.

FIG. 64 shows the percent of NK cells with high GFP expression (GFP++) following editing to insert GFP at the AAVS1 locus.

FIG. 65A shows the mean percent editing at AAVS1 assessed by NGS following treatment with LNP and varying doses of DNAPK inhibitors Compound 1 or Compound 4.

FIG. 65B shows the percent of NK cells with high GFP expression (GFP++) following editing to insert GFP at the AAVS1 locus with DNAPK inhibitor Compound 1 or Compound 4.

FIG. 66 shows relative Cas9 protein expression in macrophage cells following editing with various lipid compositions relative to Lipid A.

FIG. 67 shows the percent of B2M negative cells representing the population of cells with effective gene disruption following editing in macrophage or monocyte cells.

FIG. 68 shows the percent editing assessed with NGS in macrophage cells following treatment with LNPs 0 to 8 days post thaw.

FIGS. 69A-B shows the mean percent of negative cells following serial LNP treatment. FIG. 69A shows the percent HLA-DR, DP, DQ negative cells representing effective disruption of the CIITA locus. FIG. 69B shows the percent B2M negative cells.

FIG. 70 shows the percentage CD68+, CD11b+, HLA-ABC− cells after editing with LNPs formulated with Lipid A or Lipid D.

### DETAILED DESCRIPTION

The present disclosure provides, e.g., platform methods of using lipid nucleic acid assembly compositions for delivering nucleic acids such as genome editing tools to a cell and for multiplex genome editing in vitro. The methods provide, for example, the ability to deliver multiple genome editing tools to a cell without significant cellular side effects. The methods also provide, for example, multiple in vitro genome edits in a single cell without significant loss of viability of the cell, whereas previous methods, e.g., using electroporation, were hampered by their toxicity to the cells. In some embodiments, the platform relates to manufacturing methods to prepare cells in vitro for subsequent therapeutic administration to a subject. In some embodiments, the platform relates to multiplex genome editing via simultaneous or sequential administration of lipid nucleic acid assembly compositions comprising genome editing tools. The platform is relevant to any cell type but is particularly advantageous in preparing cells that require multiple genome edits for full therapeutic applicability, e.g., in primary immune cells. The methods may exhibit improved properties as compared to prior delivery technologies, for example, the methods provide efficient delivery of nucleic acids such as the genome editing tools, while reducing loss of cell viability and/or cell death caused by the transfection process itself, e.g., due to high levels of DNA damage, including translocations, caused by prior transfection methods. As provided herein, the platform methods apply to “a cell” in vitro or to “a cell population” (or “population of cells”) in vitro. When referring to delivery or gene editing methods for “a cell” herein, it is understood that the methods may be used for delivery or gene editing to “a cell population.”

In some embodiments, provided herein is a method of delivering two or more lipid nucleic acid assembly compositions comprising nucleic acids, e.g., genome editing tools to a cell in vitro. In some embodiments, the method comprises administering the multiple nucleic acid assembly compositions sequentially and/or simultaneously. In some embodiments, the method comprises preincubating a serum factor with the lipid nucleic acid assembly composition. In some embodiments, the lipid nucleic acid assembly composition comprises a nucleic acid, an amine lipid, a helper lipid, a neutral lipid, and a PEG lipid. In some embodiments, the method further comprises contacting the cell with the preincubated lipid nucleic acid assembly composition in vitro. In some embodiments, the method further comprises culturing the cell in vitro. In some embodiments, the method results in the delivery of the genome editing tools to the cell without significant loss of viability of the cell.

In some embodiments, provided herein is a method of producing a genetically engineered primary immune cell, e.g., T cell or B cell, in vitro. In some embodiments, the primary immune cell is cultured in vitro and provided a lipid nucleic acid assembly composition comprising a nucleic acid genome editing tool. In some embodiments, the primary immune cell is provided more than one such composition. In some embodiments, the method results in the production of a genetically engineered primary immune cell. In some embodiments, the method results in the production of a genetically engineered primary immune cell with more than one genetic modification.

In some embodiments, provided herein are methods that utilize lipid nucleic acid assemblies, e.g. lipid nanoparticle (LNP)-based compositions, with useful properties, in particular for delivery of CRISPR-Cas gene editing components. The lipid nucleic acid assembly compositions facilitate delivery of nucleic acids across cell membranes, and in particular embodiments, they introduce components and compositions for gene editing into living cells. In some embodiments, the methods provide delivery of a guide RNA with an RNA-guided DNA binding agent such as the CRISPR-Cas system via, e.g. an LNP composition, to substantially reduce or knockout expression of a specific gene. In some embodiments, the methods provide delivery of a guide RNA with an RNA-guided DNA binding agent, such as the CRISPR-Cas system, via a lipid nucleic acid assembly such as an LNP composition, and a donor nucleic acid (also referred to herein as a “template nucleic acid” or an “exogenous nucleic acid”), e.g. DNA encoding a desired protein that may be inserted into a target sequence. Some embodiments do both.

Methods to deliver components of CRISPR/Cas gene editing systems to immune cells such as mononuclear cells, including lymphocytes, and particularly T cells, in culture are of particular interest. Methods of delivering RNAs, including CRISPR/Cas system components to immune cells such as mononuclear cells, including lymphocytes, and particularly T cells, are provided herein. The methods deliver nucleic acid to the cells, including to lymphocytes, and particularly T cells, cultured in vitro and include contacting the cells with a lipid nanoparticle (LNP) composition that provides an mRNA that encodes the protein. In addition, methods of gene editing in immune cells, e.g. lymphocytes, and particularly T cells, in vitro, and methods of producing an engineered cell are provided.

In some embodiments, provided herein are compositions of cell populations comprising edited cells. In some embodiments, such cell populations comprise edited cells comprising multiple genome edits per cell. The disclosure provides for cell populations comprising edited cells, wherein the population of cells comprises edited cells comprising a single genome edit. In some embodiments, the disclosure provides for cell populations comprising edited cells comprising at least two genome edits. In some embodiments, the cell populations comprising edited cells e.g., have low levels of translocations, e.g., are capable of expansion after initiation of editing, and are suitable as a cell therapy product.

In some embodiments, described herein are compositions and methods for adoptive cell transfer (ACT) therapies, such as for immunooncology, for example, cells modified at one or more specific target sequences in their genome, including as modified by introduction of CRISPR systems that include gRNA molecules which target said target sequences, and methods of making and using thereof. For example, the present disclosure relates to and provides gRNA molecules, CRISPR systems, cells, and methods useful for genome editing of immune cells, e.g., T cells engineered to lack endogenous TCR expression, e.g., T cells suitable for further engineering to insert a nucleic acid of interest, e.g., T cells further engineered to express a TCR, such as a transgenic TCR (tgTCR), and useful for ACT therapies; and for genome editing of B cells, e.g., B cells engineered to lack endogenous B cell receptor (BCR) expression, e.g., B cells suitable for further engineering to insert a nucleic acid of interest, e.g., B cells further engineered to express a BCR, such as a transgenic BCR (tgBCR), or for expression of an antibody; NK cells or monocytes or macrophages or iPSC, or primary cells, or progenitor cells disclosed herein engineered to lack endogenous molecules e.g., for improved suitability for ACT therapies, e.g., NK cells or monocytes or macrophages or iPSC, or primary cells, or progenitor cells disclosed herein suitable for engineering to insert a nucleic acid of interest, e.g., NK cells or monocytes or macrophages or iPSC, or primary cells, or progenitor cells disclosed herein further engineered to express a heterologous protein sequence, and useful for ACT therapies.

In some embodiments, the methods provide new processes for genetically engineering T cells useful as adoptive cell therapies. For example, in some embodiments a T cell is genetically modified in vitro to reduce expression of multiple target genes, including e.g., endogenous T cell receptor genes, among others, and further modified to insert a transgenic TCR in the form of a donor nucleic acid. In some embodiments, T cells particularly desirable for use as adoptive cell therapies require multiple gene edits. The ability to genetically engineer a T cell in vitro with the sort of multitude of modifications to the genome disclosed herein has previously proven a technical challenge. In addition to the hurdles associated with multiplex gene editing discussed above, T cells are particularly challenging to genetically modify in culture and can become exhausted, for example.

Provided herein are methods for genetically engineering T cells in vitro that overcome the hurdles of prior processes. In some embodiments, naïve T cells are contacted in vitro with at least one lipid nucleic acid assembly composition and genetically modified. In some embodiments, non-activated T cells are contacted in vitro with two or more lipid nucleic acid assembly compositions and genetically modified. In some embodiments, activated T cells are contacted in vitro with two or more lipid nucleic acid assembly compositions and genetically modified. In some embodiments, T cells are modified in a pre-activation step, comprising contacting the (non-activated) T cell with one or more lipid nucleic acid assembly compositions, followed by activating the T cell, followed by further modifications to the T cell in a post-activation step, comprising contacting the activated T cell with one or more lipid nucleic acid assembly compositions. In some embodiments, the non-activated T cell is contacted with one, two, or three lipid nucleic acid assembly compositions. In some embodiments, the activated T cell is contacted with one to 12 lipid nucleic acid assembly compositions. In some embodiments, the activated T cell is contacted with one to 8 lipid nucleic acid assembly compositions, optionally 1 to 4 lipid nucleic acid assembly compositions. In some embodiments, the activated T cell is contacted with one to 6 lipid nucleic acid assembly compositions. In some embodiments, the T cell is contacted with two lipid nucleic acid assembly compositions. In some embodiments, the T cell is contacted with three lipid nucleic acid assembly compositions. In some embodiments, the T cell is contacted with four lipid nucleic acid assembly compositions. In some embodiments, the T cell is contacted with five lipid nucleic acid assembly compositions. In some embodiments, the T cell is contacted with six lipid nucleic acid assembly compositions. In some embodiments, the T cell is contacted with seven lipid nucleic acid assembly compositions. In some embodiments, the T cell is contacted with eight lipid nucleic acid assembly compositions. In some embodiments, the T cell is contacted with nine lipid nucleic acid assembly compositions. In some embodiments, the T cell is contacted with ten lipid nucleic acid assembly compositions. In some embodiments, the T cell is contacted with eleven lipid nucleic acid assembly compositions. In some embodiments, the T cell is contacted with twelve lipid nucleic acid assembly compositions. Such exemplary sequential administration (optionally with further sequential or simultaneous administration in the pre-activation step and post-activation step) of lipid nucleic acid assembly compositions takes advantage of the activation status of the T cell and provides for unique advantages and healthier cells post-editing. In some embodiments, the genetically engineered T cells have the advantageous properties of high editing efficiency at each target site, increased post-editing survival rate, low toxicity despite the multiplicity of transfections, low translocations (e.g., no measurable target-target translocations), increased production of cytokines (e.g., IL-2, IFNγ, TNFα), continued proliferation with repeat stimulation (e.g., with repeat antigen stimulation), increased expansion, expression of memory cell phenotype markers, including for examples, early stem cell.

### I. DEFINITIONS

Unless stated otherwise, the following terms and phrases as used herein are intended to have the following meanings:

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “gRNA”, and simply “guide” are used herein interchangeably to refer to the guide that directs an RNA-guided DNA binding agent to a target DNA and can be either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally occurring sequence, or a trRNA sequence with modifications or variations compared to naturally-occurring sequences.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Target sequences for RNA-guided DNA binding agents include both the positive and negative strands of genomic DNA (i.e., the sequence given and the sequence's reverse compliment), as a nucleic acid substrate for an RNA-guided DNA binding agent is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease”, also called “Cas protein” as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015).

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

As used herein, the term “editor” refers to an agent comprising a polypeptide that is capable of making a modification to a base (e.g., A, T, C, G, or U) within a nucleic acid sequence (e.g., DNA or RNA). In some embodiments, the editor is capable of deaminating a base within a nucleic acid. In some embodiments, the editor is capable of deaminating a base within a DNA molecule. In some embodiments, the editor is capable of deaminating a cytosine (C) in DNA. In some embodiments, the editor is a fusion protein comprising an RNA-guided nickase fused to a cytidine deaminase domain. In some embodiments, the editor is a fusion protein comprising an RNA-guided nickase fused to an APOBEC3A deaminase (A3A). In some embodiments, the editor comprises a Cas9 nickase fused to an APOBEC3A deaminase (A3A).

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

As used herein, “indels” refer to insertion/deletion mutations consisting of a number of nucleotides that are either inserted or deleted, e.g., at the site of double-stranded breaks (DSBs) in a target nucleic acid.

As used herein, “knockdown” refers to a decrease in expression of a particular gene product (e.g., protein, mRNA, or both). Knockdown of a protein can be measured by detecting total cellular amount of the protein from a sample, such as a tissue, fluid, or cell population of interest. It can also be measured by measuring a surrogate, marker, or activity for the protein. Methods for measuring knockdown of mRNA are known and include sequencing of mRNA isolated from a sample of interest. In some embodiments, “knockdown” may refer to some loss of expression of a particular gene product, for example a decrease in the amount of mRNA transcribed or a decrease in the amount of protein expressed by a population of cells (including in vivo populations such as those found in tissues).

As used herein, “knockout” refers to a loss of expression from a particular gene or of a particular protein in a cell. Knockout can be measured either by detecting total cellular amount of a protein in a cell, a tissue or a population of cells.

As used herein, a “target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, “treatment” refers to any administration or application of a therapeutic for disease or disorder in a subject, and includes inhibiting the disease, arresting its development, relieving one or more symptoms of the disease, curing the disease, or preventing one or more symptoms of the disease, including reoccurrence of the symptom.

As used herein, a “cell population comprising edited cells,” or a “population of cells comprising edited cells,” or the like refers to a cell population that comprises edited cells, however not all cells in the population must be edited. A cell population comprising edited cells may also include non-edited cells. The percentage of edited cells within a cell population comprising edited cells may be determined by counting the number of cells within the population that are edited in the population as determined by standard cell counting methods. For example, in some embodiments, a cell population comprising edited cells comprising a single genome edit will have at least 20%, 30%, 40%, 50%, 60%, 70%, 80%, 90%, 95%, or 99% of the cells in the population with the single edit. In some embodiments, a cell population comprising edited cells comprising at least two genome edits will have at least 20%, 30%, 40%, 50%, 60%, 70%, 80%, 90%, or 95% of the cells in the population with at least two genome edits.

The term “about” or “approximately” means an acceptable error for a particular value as determined by one of ordinary skill in the art, which depends in part on how the value is measured or determined, or a degree of variation that does not substantially affect the properties of the described subject matter, or within the tolerances accepted in the art, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### II. MULTIPLEX DELIVERY AND GENOME EDITING

A. Multiplex Delivery

In some embodiments, methods of delivering multiple lipid nucleic acid assembly compositions to a cell in vitro are provided. In some embodiments, the multiplex delivery method results in a cell that is capable of expanding into a cell population. In some embodiments, expansion of the cell into a cell population is a marker of successful multiplex delivery. Similarly, methods of delivering multiple lipid nucleic acid assembly compositions to a cell in vitro to produce an expanded cell population having increased survival are provided. Such methods are useful, for example, in producing/manufacturing cells to be used in cell therapy, which, as used herein, refers to the transfer of live, intact cells into a subject to treat a disease or disorder. Cell therapy approaches such as transplantation of therapeutic cells including ACT therapies are included. Cell therapy includes autologous (cells originating from the subject) and allogenic (cells originating from a donor) cell therapy.

In some embodiments, the multiplex delivery method comprises delivering at least two lipid nucleic acid assembly compositions to an in vitro-cultured cell. In some embodiments, a cell in vitro is contacted with at least a first lipid nucleic acid assembly composition comprising a first nucleic acid, thereby producing a contacted cell, the contacted cell is cultured thereby producing a cultured contacted cell, and the cultured contacted cell is contacted with at least a second lipid nucleic acid assembly composition comprising a second nucleic acid, wherein the second nucleic acid is different from the first nucleic acid. The resulting cell is then expanded in vitro. In some embodiments, the delivery method results in an expanded cell population, such as a cell population having increased survival. In some embodiments, the expanded cell has a survival rate of at least 70%. The “first” and “second” nucleic acid may comprise guide RNAs (gRNA).

In some embodiments, methods are provided for delivering lipid nucleic acid assembly compositions to an in vitro-cultured cell, comprising the steps of: a) contacting the cell in vitro with at least a first lipid nucleic acid assembly composition comprising a first nucleic acid, thereby producing a contacted cell; b) culturing the contacted cell in vitro, thereby producing a cultured contacted cell; c) contacting the cultured contacted cell in vitro with at least a second lipid nucleic acid assembly composition comprising a second nucleic acid, wherein the second nucleic acid is different from the first nucleic acid; and d) expanding the cell in vitro; wherein the expanded cell exhibits increased survival. In some embodiments, the expanded cell has a survival rate of at least 70%. In some embodiments, the cell is contacted with 2-12 lipid nucleic acid assembly compositions. In some embodiments, the cell is contacted with 2-8 lipid nucleic acid assembly compositions. In some embodiments, the cell is contacted with 2-6 lipid nucleic acid assembly compositions. In some embodiments, the cell is contacted with 3-8 lipid nucleic acid assembly compositions. In some embodiments, the cell is contacted with 3-6 lipid nucleic acid assembly compositions. In some embodiments, the cell is contacted with 4-6 lipid nucleic acid assembly compositions. In some embodiments, the cell is contacted with 4-12 lipid nucleic acid assembly compositions. In some embodiments, the cell is contacted with 4-8 lipid nucleic acid assembly compositions. In some embodiments, the cell is contacted with 6-12 lipid nucleic acid assembly compositions. In some embodiments, the cell is contacted with 3, 4, 5, or 6 lipid nucleic acid assembly compositions. In some embodiments, the cell is contacted with 6 lipid nucleic acid assembly compositions. In some embodiments, the cell is contacted with no more than 8 lipid nucleic acid assembly compositions simultaneously. In some embodiments, the cell is contacted with no more than 6 lipid nucleic acid assembly compositions simultaneously. In some embodiments, the cell is a T cell. In some embodiments, the cell is a non-activated cell. In some embodiments, the cell is an activated cell. In some embodiments, the cell of (a) is activated after contact with at least one lipid nucleic acid assembly composition.

In some embodiments, an “increased survival” is demonstrated by a post-transfection cell survival rate, or cell survival rate of the expanded cell, or cells, of at least 60%, at least 70%, at least 80%, at least 90%, or at least 95% (referring to the viability of the population of cells comprising edited cells resulting from the expanded cell). In some embodiments, the lipid nucleic acid assembly methods may reduce cell death as compared to known technologies like electroporation. In some embodiments, the lipid nucleic acid assembly methods may cause less than 5%, less than 10%, less than 20%, less than 30%, or less than 40% cell death. In some embodiments, the lipid nucleic acid assembly methods deliver a nucleic acid such as RNA without significant loss of viability of the cell, whereas previous methods, e.g., using electroporation, were hampered by their toxicity to the cells. In some embodiments, the lipid nucleic acid assembly methods result in cell expansion and/or cell phenotype improvements, such as engineered T cell populations with a favorable early-stem cell memory phenotype, cytokine production, proliferation profile following repeated antigen stimulation, and/or chromosomal translocation rate.

In some embodiments, the cell is contacted with 1, 2, 3, 4, 5, 6, 7, 8, 9, 10, or 11 lipid nucleic acid assembly compositions. In some embodiments, the cell is contacted with at least 6 lipid nucleic acid assembly compositions. In some embodiments, the cell is contacted with no more than 12 lipid nucleic acid assembly compositions. In some embodiments, the cell is contacted with 2-12 lipid nucleic acid assembly compositions. In some embodiments, the cell is contacted with 2-8 lipid nucleic acid assembly compositions. In some embodiments, the cell is contacted with 2-6 lipid nucleic acid assembly compositions. In some embodiments, the cell is contacted with 3-8 lipid nucleic acid assembly compositions. In some embodiments, the cell is contacted with 3-6 lipid nucleic acid assembly compositions. In some embodiments, the cell is contacted with 4-6 lipid nucleic acid assembly compositions. In some embodiments, the cell is contacted with 4-12 lipid nucleic acid assembly compositions. In some embodiments, the cell is contacted with 4-8 lipid nucleic acid assembly compositions. In some embodiments, the cell is contacted with 6-12 lipid nucleic acid assembly compositions. In some embodiments, the cell is contacted with 3, 4, 5, or 6 lipid nucleic acid assembly compositions. In some embodiments, the cell is contacted with no more than 8 lipid nucleic acid assembly compositions simultaneously. In some embodiments, the cell is contacted with no more than 6 lipid nucleic acid assembly compositions simultaneously.

In some embodiments, the cell is contacted with two lipid nucleic acid assembly compositions. In some embodiments, the cell is contacted with three lipid nucleic acid assembly compositions. In some embodiments, the cell is contacted with four lipid nucleic acid assembly compositions. In some embodiments, the cell is contacted with five lipid nucleic acid assembly compositions. In some embodiments, the cell is contacted with six lipid nucleic acid assembly compositions.

In some embodiments, the contact between the cell and lipid nucleic acid assembly composition is sequential (one following another). In some embodiments, the contact between the cell and lipid nucleic acid assembly composition is simultaneous (contacts are concurrent or nearly concurrent). In some embodiments, the multiple lipid nucleic acid assembly compositions are administered sequentially. In some embodiments, the lipid nucleic acid assembly compositions are administered simultaneously. In some embodiments, the lipid nucleic acid assembly compositions are administered sequentially and simultaneously. For example, in some embodiments, three lipid nucleic acid compositions are provided and two lipid nucleic acid compositions are administered first simultaneously, the cell is cultured for some period of time, and then the third lipid nucleic acid composition is administered (i.e., sequentially, after the administration of the first two composition). In another embodiment, three lipid nucleic acid compositions are provided and one lipid nucleic acid composition is administered first, the cell is cultured for some period of time, and then two lipid nucleic acid composition are administered simultaneously (and sequentially, after the administration of the first composition). Thus, simultaneous and sequential administration of lipid nucleic acid assembly composition may overlap in certain embodiments.

In some embodiments, methods are provided for delivering lipid nucleic acid assembly compositions to an in vitro-cultured cell, comprising the steps of: a) contacting the cell in vitro with at least a first lipid nucleic acid assembly composition comprising a first nucleic acid, thereby producing a contacted cell; b) culturing the contacted cell in vitro, thereby producing a cultured contacted cell; c) contacting the cultured contacted cell in vitro with at least a second lipid nucleic acid assembly composition comprising a second nucleic acid, wherein the second nucleic acid is different from the first nucleic acid; and d) expanding the cell in vitro; wherein the expanded cell exhibits increased survival, wherein the cell is contacted with at least six lipid nucleic acid assembly compositions. In some embodiments, the expanded cell has a survival rate of at least 70%. In some embodiments, at least four lipid nucleic acid assembly compositions comprise a guide RNA, and at least one lipid nucleic acid assembly composition comprises a first genome editing tool, thereby producing multiple genome edits in the cell. In some embodiments, the at least six lipid nucleic acid assembly compositions are administered simultaneously. In some embodiments, the first genome editing tool is an RNA-guided DNA binding agent. In some embodiments, the RNA-guided DNA binding agent is a Cas9. In some embodiments, the RNA-guided DNA binding agent comprises a APOBEC3A deaminase (A3A) and an RNA-guided nickase. In some embodiments, the method comprises contacting the cell with a lipid nucleic acid composition comprising a second genome editing tool. In some embodiments, the second genome editing tool is a UGI. In some embodiments, the second genome editing tool is a donor nucleic acid. In some embodiments, the method comprises contacting the cell with a lipid nucleic acid composition comprising a third genome editing tool. In some embodiments, the third genome editing tool is an RNA-guided DNA binding agent. In some embodiments, the third genome editing tool is a UGI. In some embodiments, the third genome editing tool is a donor nucleic acid. In some embodiments, the genome editing tool (e.g., first genome editing tool, second genome editing tool, third genome editing tool) is mRNA. In some embodiments, the cell is a T cell. In some embodiments, the cell is a non-activated cell. In some embodiments, the cell is an activated cell. In some embodiments, the cell of (a) is activated after contact with at least one lipid nucleic acid assembly composition.

In some embodiments, methods are provided for delivering lipid nanoparticle (LNP) compositions to a population of in vitro cultured cells, comprising the steps of: a) contacting the population of cells in vitro with at least a first LNP composition comprising a first nucleic acid, thereby producing a contacted population of cells; b) culturing the contacted population of cells in vitro, thereby producing a population of cultured contacted cells; c) contacting the population of cells or the population of cultured contacted cells in vitro with at least a second LNP composition comprising a second nucleic acid, wherein the second nucleic acid is different from the first nucleic acid; and d) expanding the population of cells in vitro; wherein the expanded population of cells exhibits a survival rate of at least 70%. In some embodiments, the expanded population of cells has a survival rate of at least 70% at 24 hours of expansion. In some embodiments, the expanded population of cells has a survival rate of at least 80% at 24 hours of expansion. In some embodiments, the expanded population of cells has a survival rate of at least 90% at 24 hours of expansion. In some embodiments, the expanded population of cells has a survival rate of at least 95% at 24 hours of expansion. In some embodiments, the population of cells and the population of cultured contacted cells is contacted with a total of 2-12 LNP compositions. In some embodiments, the population of cells and the population of cultured contacted cells is contacted with a total of 2-8 LNP compositions. In some embodiments, the population of cells and the population of cultured contacted cells is contacted with a total of 2-6 LNP compositions. In some embodiments, the population of cells and the population of cultured contacted cells is contacted with a total of 3-8 LNP compositions. In some embodiments, the population of cells and the population of cultured contacted cells is contacted with a total of 3-6 LNP compositions. In some embodiments, the population of cells and the population of cultured contacted cells is contacted with a total of 4-6 LNP compositions. In some embodiments, the population of cells and the population of cultured contacted cells is contacted with a total of 6-12 LNP compositions. In some embodiments, the population of cells and the population of cultured contacted cells is contacted with a total of 3 LNP compositions. In some embodiments, the population of cells and the population of cultured contacted cells is contacted with a total of 4 LNP compositions. In some embodiments, the population of cells and the population of cultured contacted cells is contacted with a total of 6 LNP compositions. In some embodiments, the population of cells and the population of cultured contacted cells is contacted with a total of 3 LNP compositions. In some embodiments, the population of cells is contacted with the LNP compositions simultaneously. In some embodiments, the population of cells is contacted with no more than 6 LNP compositions simultaneously. In some embodiments, the population of cells is contacted with no more than 2 LNP compositions simultaneously.

In some embodiments, methods are provided for delivering lipid nanoparticle (LNP) compositions to a population of in vitro cultured cells, comprising the steps of: a) contacting the population of cells in vitro with at least a first LNP composition comprising a first nucleic acid, thereby producing a contacted population of cells; b) culturing the contacted population of cells in vitro, thereby producing a population of cultured contacted cells; c) contacting the population of cells or the population of cultured contacted cells in vitro with at least a second LNP composition comprising a second nucleic acid, wherein the second nucleic acid is different from the first nucleic acid; and d) expanding the population of cells in vitro; wherein at least 70%, 80%, 90%, or 95% of the cells in the population of cells are viable 24 hours after the last contact with an LNP composition. In some embodiments, at least 70% of the cells in the population of cells are viable 24 hours after the last contact with an LNP composition. In some embodiments, at least 80% of the cells in the population of cells are viable 24 hours after the last contact with an LNP composition. In some embodiments, at least 90% of the cells in the population of cells are viable 24 hours after the last contact with an LNP composition. In some embodiments, at least 95% of the cells in the population of cells are viable 24 hours after the last contact with an LNP composition. In some embodiments, the population of cells and the population of cultured contacted cells is contacted with a total of 2-12 LNP compositions. In some embodiments, the population of cells and the population of cultured contacted cells is contacted with a total of 2-8 LNP compositions. In some embodiments, the population of cells and the population of cultured contacted cells is contacted with a total of 2-6 LNP compositions. In some embodiments, the population of cells and the population of cultured contacted cells is contacted with a total of 3-8 LNP compositions. In some embodiments, the population of cells and the population of cultured contacted cells is contacted with a total of 3-6 LNP compositions. In some embodiments, the population of cells and the population of cultured contacted cells is contacted with a total of 4-6 LNP compositions. In some embodiments, the population of cells and the population of cultured contacted cells is contacted with a total of 6-12 LNP compositions. In some embodiments, the population of cells and the population of cultured contacted cells is contacted with a total of 3 LNP compositions. In some embodiments, the population of cells and the population of cultured contacted cells is contacted with a total of 4 LNP compositions. In some embodiments, the population of cells and the population of cultured contacted cells is contacted with a total of 6 LNP compositions. In some embodiments, the population of cells and the population of cultured contacted cells is contacted with a total of 3 LNP compositions. In some embodiments, the population of cells is contacted with the LNP compositions simultaneously. In some embodiments, the population of cells is contacted with no more than 6 LNP compositions simultaneously. In some embodiments, the population of cells is contacted with no more than 2 LNP compositions simultaneously.

In some embodiments, methods are provided for delivering lipid nucleic acid assembly compositions to an in vitro-cultured cell, comprising the steps of: a) contacting the cell in vitro with at least a first lipid nucleic acid assembly composition comprising a first nucleic acid, thereby producing a contacted cell; b) culturing the contacted cell in vitro, thereby producing a cultured contacted cell; c) contacting the cultured contacted cell in vitro with at least a second lipid nucleic acid assembly composition comprising a second nucleic acid, wherein the second nucleic acid is different from the first nucleic acid; and d) expanding the cell in vitro; wherein the expanded cell exhibits increased survival, wherein one of the lipid nucleic acid assembly compositions comprises a gRNA targeting TRAC. In some embodiments, one of the lipid nucleic acid assembly compositions comprises a gRNA targeting TRBC. In some embodiments, one of the lipid nucleic acid assembly compositions comprises a gRNA targeting a gene that reduces or eliminates surface expression of MHC class I. In some embodiments, one of the lipid nucleic acid assembly compositions comprises a gRNA targeting B2M. In some embodiments, one of the lipid nucleic acid assembly compositions comprises a gRNA targeting HLA-A, optionally wherein the cell is homozygous for HLA-B and homozygous for HLA-C. In some embodiments, one of the lipid nucleic acid assembly compositions comprises a gRNA targeting a gene that reduces or eliminates surface expression of MHC class II. In some embodiments, one of the lipid nucleic acid assembly compositions comprises a gRNA targeting CIITA.

In some embodiments, methods are provided for delivering lipid nucleic acid assembly compositions to an in vitro-cultured cell, comprising the steps of: a) contacting the cell in vitro with at least a first lipid nucleic acid assembly composition comprising a first nucleic acid, thereby producing a contacted cell; b) culturing the contacted cell in vitro, thereby producing a cultured contacted cell; c) contacting the cultured contacted cell in vitro with at least a second lipid nucleic acid assembly composition comprising a second nucleic acid, wherein the second nucleic acid is different from the first nucleic acid; and d) expanding the cell in vitro; wherein the expanded cell exhibits increased survival, wherein the first and second lipid nucleic acid compositions each comprise a gRNA selected from a) a gRNA targeting TRAC, b) a gRNA targeting TRBC, c) a gRNA targeting B2M or a gRNA targeting HLA-A, and d) a gRNA targeting CIITA. In some embodiments, a further lipid nucleic acid assembly composition comprises an RNA-guided DNA binding agent. In some embodiments, the RNA-guided DNA binding agent is Cas9. In some embodiments, a further lipid nucleic acid assembly composition comprises a donor nucleic acid.

In some embodiments, methods are provided for delivering lipid nucleic acid assembly compositions to an in vitro-cultured cell, comprising the steps of: a) contacting the cell in vitro with at least a first lipid nucleic acid assembly composition comprising a first nucleic acid, thereby producing a contacted cell; b) culturing the contacted cell in vitro, thereby producing a cultured contacted cell; c) contacting the cultured contacted cell in vitro with at least a second lipid nucleic acid assembly composition comprising a second nucleic acid, wherein the second nucleic acid is different from the first nucleic acid; and d) expanding the cell in vitro; wherein the expanded cell exhibits increased survival, wherein one of the lipid nucleic acid assembly composition comprises a gRNA targeting TRAC, and one of the lipid nucleic acid assembly compositions comprises a gRNA targeting TRBC. In some embodiments, a further lipid nucleic acid assembly composition comprises an RNA-guided DNA binding agent. In some embodiments, the RNA-guided DNA binding agent is Cas9. In some embodiments, a further lipid nucleic acid assembly composition comprises a donor nucleic acid.

In some embodiments, methods are provided for delivering lipid nucleic acid assembly compositions to an in vitro-cultured cell, comprising the steps of: a) contacting the cell in vitro with at least a first lipid nucleic acid assembly composition comprising a first nucleic acid, thereby producing a contacted cell; b) culturing the contacted cell in vitro, thereby producing a cultured contacted cell; c) contacting the cultured contacted cell in vitro with at least a second lipid nucleic acid assembly composition comprising a second nucleic acid, wherein the second nucleic acid is different from the first nucleic acid; and d) expanding the cell in vitro; wherein the expanded cell exhibits increased survival, wherein one of the lipid nucleic acid assembly compositions comprises a gRNA targeting TRAC, one of the lipid nucleic acid assembly compositions comprises a gRNA targeting TRBC, and a further lipid nucleic acid assembly composition comprises a gRNA targeting B2M. In some embodiments, a further lipid nucleic acid assembly composition comprises an RNA-guided DNA binding agent. In some embodiments, the RNA-guided DNA binding agent is Cas9. In some embodiments, a further lipid nucleic acid assembly composition comprises a donor nucleic acid.

In some embodiments, methods are provided for delivering lipid nucleic acid assembly compositions to an in vitro-cultured cell, comprising the steps of: a) contacting the cell in vitro with at least a first lipid nucleic acid assembly composition comprising a first nucleic acid, thereby producing a contacted cell; b) culturing the contacted cell in vitro, thereby producing a cultured contacted cell; c) contacting the cultured contacted cell in vitro with at least a second lipid nucleic acid assembly composition comprising a second nucleic acid, wherein the second nucleic acid is different from the first nucleic acid; and d) expanding the cell in vitro; wherein the expanded cell exhibits increased survival, wherein one of the lipid nucleic acid assembly compositions comprises a gRNA targeting TRAC, one of the lipid nucleic acid assembly compositions comprises a gRNA targeting TRBC, and a further lipid nucleic acid assembly composition comprises a gRNA targeting HLA-A, optionally wherein the cell is homozygous for HLA-B and homozygous for HLA-C. In some embodiments, a further lipid nucleic acid assembly composition comprises an RNA-guided DNA binding agent. In some embodiments, the RNA-guided DNA binding agent is Cas9. In some embodiments, a further lipid nucleic acid assembly composition comprises a donor nucleic acid.

In some embodiments, methods are provided for delivering lipid nucleic acid assembly compositions to an in vitro-cultured cell, comprising the steps of: a) contacting the cell in vitro with at least a first lipid nucleic acid assembly composition comprising a first nucleic acid, thereby producing a contacted cell; b) culturing the contacted cell in vitro, thereby producing a cultured contacted cell; c) contacting the cultured contacted cell in vitro with at least a second lipid nucleic acid assembly composition comprising a second nucleic acid, wherein the second nucleic acid is different from the first nucleic acid; and d) expanding the cell in vitro; wherein the expanded cell exhibits increased survival, wherein one of the lipid nucleic acid assembly compositions comprises a gRNA targeting TRAC, one of the lipid nucleic acid assembly compositions comprises a gRNA targeting TRBC, a further lipid nucleic acid assembly composition comprises a gRNA targeting B2M, and a further lipid nucleic acid assembly composition comprises a gRNA targeting CIITA. In some embodiments, a further lipid nucleic acid assembly composition comprises an RNA-guided DNA binding agent. In some embodiments, the RNA-guided DNA binding agent is Cas9. In some embodiments, a further lipid nucleic acid assembly composition comprises a donor nucleic acid.

In some embodiments, methods are provided for delivering lipid nucleic acid assembly compositions to an in vitro-cultured cell, comprising the steps of: a) contacting the cell in vitro with at least a first lipid nucleic acid assembly composition comprising a first nucleic acid, thereby producing a contacted cell; b) culturing the contacted cell in vitro, thereby producing a cultured contacted cell; c) contacting the cultured contacted cell in vitro with at least a second lipid nucleic acid assembly composition comprising a second nucleic acid, wherein the second nucleic acid is different from the first nucleic acid; and d) expanding the cell in vitro; wherein the expanded cell exhibits increased survival, wherein one of the lipid nucleic acid assembly compositions comprises a gRNA targeting TRAC, one of the lipid nucleic acid assembly compositions comprises a gRNA targeting TRBC, a further lipid nucleic acid assembly composition comprises a gRNA targeting HLA-A, optionally wherein the cell is homozygous for HLA-B and homozygous for HLA-C, and a further lipid nucleic acid assembly composition comprises a gRNA targeting CIITA. In some embodiments, a further lipid nucleic acid assembly composition comprises an RNA-guided DNA binding agent. In some embodiments, the RNA-guided DNA binding agent is Cas9. In some embodiments, a further lipid nucleic acid assembly composition comprises a donor nucleic acid.

In some embodiments, the donor nucleic acid encodes a targeting receptor. A “targeting receptor” is a polypeptide present on the surface of a cell, e.g., a T cell, to permit binding of the cell to a target site, e.g., a specific cell or tissue in an organism. In some embodiments, the targeting receptor is a CAR. In some embodiments, the targeting receptor is a universal CAR (UniCAR). In some embodiments, the targeting receptor is a TCR. In some embodiments, the targeting receptor is a T cell receptor fusion construct (TRuC). In some embodiments, the targeting receptor is a B cell receptor (BCR) (e.g., expressed on a B cell). In some embodiments, the targeting receptor is chemokine receptor. In some embodiments, the targeting receptor is a cytokine receptor.

β2M or B2M are used interchangeably herein and with reference to nucleic acid sequence or protein sequence of β-2 microglobulin; the human gene has accession number NC_000015 (range 44711492 . . . 44718877), reference GRCh38.p13. The B2M protein is associated with MEW class I molecules as a heterodimer on the surface of nucleated cells and is required for MHC class I protein expression.

CITTA or CIITA or C2TA are used interchangeably herein and with reference to the nucleic acid sequence or protein sequence of class II major histocompatibility complex transactivator; the human gene has accession number NC_000016.10 (range 10866208 . . . 10941562), reference GRCh38.p13. The CIITA protein in the nucleus acts as a positive regulator of MEW class II gene transcription and is required for MEW class II protein expression.

MEW or MEW molecule(s) or MEW protein or MHC complex(es), refer to a major histocompatibility complex molecule (or plural), and include e.g., MHC class I and MEW class II molecules. In humans, MEW molecules are referred to as human leukocyte antigen complexes or HLA molecules or HLA protein. The use of terms MEW and HLA are not meant to be limiting; as used herein, the term MHC may be used to refer to human MEW molecules, i.e., HLA molecules. Therefore, the terms MHC and HLA are used interchangeably herein.

HLA-A as used herein in the context of HLA-A protein, refers to the MEW class I protein molecule, which is a heterodimer consisting of a heavy chain (encoded by the HLA-A gene) and a light chain (i.e., beta-2 microglobulin). The terms HLA-A or HLA-A gene, as used herein in the context of nucleic acids refers to the gene encoding the heavy chain of the HLA-A protein molecule. The HLA-A gene is also referred to as HLA class I histocompatibility, A alpha chain; the human gene has accession number NC_000006.12 (29942532 . . . 29945870). The HLA-A gene is known to have hundreds of different versions (also referred to as alleles) across the population (and an individual may receive two different alleles of the HLA-A gene). All alleles of HLA-A are encompassed by the terms HLA-A and HLA-A gene.

HLA-B as used herein in the context of nucleic acids refers to the gene encoding the heavy chain of the HLA-B protein molecule. The HLA-B is also referred to as HLA class I histocompatibility, B alpha chain; the human gene has accession number NC_000006.12 (31353875 . . . 31357179).

HLA-C as used herein in the context of nucleic acids refers to the gene encoding the heavy chain of the HLA-C protein molecule. The HLA-C is also referred to as HLA class I histocompatibility, C alpha chain; the human gene has accession number NC_000006.12 (31268749 . . . 31272092).

The term homozygous refers to having two identical alleles of a particular gene.

Any cell type described herein may be used in the delivery methods. Cells useful for ACT therapies such as stem, progenitor, and primary cells are included.

In some embodiments, the lipid nucleic acid assembly composition is pretreated with a serum factor before contacting the cell. In some embodiments, the lipid nucleic acid assembly composition is pretreated with a human serum before contacting the cell. In some embodiments, the lipid nucleic acid assembly composition is pretreated with ApoE before contacting the cell. In some embodiments, the lipid nucleic acid assembly composition is pretreated with a recombinant ApoE3 or ApoE4 before contacting the cell. In some embodiments, the cell is serum-starved prior to contact with the lipid nucleic acid assembly composition.

In some embodiments, the multiplex methods comprise preincubating a serum factor and the lipid nucleic acid assembly composition for about 30 seconds to overnight. In some embodiments, the preincubation step comprises preincubating a serum factor and the lipid nucleic acid assembly composition for about 1 minute to 1 hour. In some embodiments, it comprises preincubating for about 1-30 minutes. In other embodiments, it comprises preincubating for about 1-10 minutes. Still further embodiments comprise preincubating for about 5 minutes.

In some embodiments, the preincubating step occurs at about 4° C. In some embodiments, the preincubating step occurs at about 25° C. In certain embodiments, the preincubating step occurs at about 37° C. The preincubating step may comprise a buffer such as sodium bicarbonate or HEPES.

B. Multiplex Genome Editing

In some embodiments, a method of producing multiple genome edits in a cell in vitro is provided (sometimes referred to herein and elsewhere as “multiplexing” or “multiplex gene editing” or “multiplex genome editing”). In some embodiments, the method comprises culturing a cell in vitro, contacting the cell with two or more lipid nucleic acid assembly compositions, wherein each lipid nucleic acid assembly composition comprises a nucleic acid genome editing tool capable of editing a target site, and expanding the cell in vitro. The method results in a cell having more than one genome edit, wherein the genome edits differ. In some embodiments, the method results in a cell having a single genome edit.

The terms “genome editing” and “gene editing” are used interchangeably herein. The terms “genome editing tool” and “gene editing tool” are also used interchangeably herein. The terms “nucleic acid genome editing tool” and “genome editing tool” may also be used interchangeably herein.

In some embodiments, methods are provided for producing multiple genome edits in an in vitro-cultured cells, comprising the steps of: a) contacting the cell in vitro with at least a first lipid nanoparticle (LNP) composition and a second LNP composition, wherein the first LNP composition comprises a first guide RNA (gRNA) directed to a first target sequence and optionally a nucleic acid genome editing tool and the second LNP composition comprises a second gRNA directed to a second target sequence different from the first target sequence and optionally a nucleic acid genome editing tool; and b) expanding the cell in vitro; thereby producing multiple genome edits in the cell. In some embodiments, the cell is contacted with at least one LNP composition comprising a genome editing tool. In some embodiments, the genome editing tool comprises a nucleic acid encoding an RNA-guided DNA binding agent. In some embodiments, the cell is further contacted with a donor nucleic acid for insertion in a target sequence. In some embodiments, the LNP compositions are administered sequentially. In some embodiments, the LNP compositions are administered simultaneously. In some embodiments, the population of cells is contacted with 2-12 LNP compositions. In some embodiments, the population of cells is contacted with 2-8 LNP compositions. In some embodiments, the population of cells is contacted with 2-6 LNP compositions. In some embodiments, the population of cells is contacted with 3-8 LNP compositions. In some embodiments, the population of cells is contacted with 3-6 LNP compositions. In some embodiments, the population of cells is contacted with 4-6 LNP compositions. In some embodiments, the population of cells is contacted with 6-12 LNP compositions. In some embodiments, the population of cells is contacted with 3 LNP compositions. In some embodiments, the population of cells is contacted with 4 LNP compositions. In some embodiments, the population of cells is contacted with 6 LNP compositions. In some embodiments, the population of cells is contacted with 3 LNP compositions. In some embodiments, the population of cells is contacted with the LNP compositions simultaneously. In some embodiments, the population of cells is contacted with no more than 6 LNP compositions simultaneously. In some embodiments, the population of cells is contacted with no more than 2 LNP compositions simultaneously.

In some embodiments, methods are provided for producing multiple genome edits in an in vitro-cultured cell, comprising the steps of: contacting the cell in vitro with at least a first lipid nanoparticle (LNP) composition and a second LNP composition, wherein the first lipid LNP composition comprises a first guide RNA (gRNA) directed to a first target sequence and optionally a nucleic acid genome editing tool and the second LNP composition comprises a second gRNA directed to a second target sequence different from the first target sequence and optionally a nucleic acid genome editing tool; and b) culturing the cell ex vivo; thereby producing multiple genome edits in the cell. In some embodiments, the cell is contacted with at least one LNP composition comprising a genome editing tool. In some embodiments, the genome editing tool comprises a nucleic acid encoding an RNA-guided DNA binding agent. In some embodiments, the cell is further contacted with a donor nucleic acid for insertion in a target sequence. In some embodiments, the LNP compositions are administered sequentially. In some embodiments, the LNP compositions are administered simultaneously. In some embodiments, the population of cells is contacted with 2-12 LNP compositions. In some embodiments, the population of cells is contacted with 2-8 LNP compositions. In some embodiments, the population of cells is contacted with 2-6 LNP compositions. In some embodiments, the population of cells is contacted with 3-8 LNP compositions. In some embodiments, the population of cells is contacted with 3-6 LNP compositions. In some embodiments, the population of cells is contacted with 4-6 LNP compositions. In some embodiments, the population of cells is contacted with 6-12 LNP compositions. In some embodiments, the population of cells is contacted with 3 LNP compositions. In some embodiments, the population of cells is contacted with 4 LNP compositions. In some embodiments, the population of cells is contacted with 6 LNP compositions. In some embodiments, the population of cells is contacted with 3 LNP compositions. In some embodiments, the population of cells is contacted with the LNP compositions simultaneously. In some embodiments, the population of cells is contacted with no more than 6 LNP compositions simultaneously. In some embodiments, the population of cells is contacted with no more than 2 LNP compositions simultaneously.

In some embodiments, methods are provided for gene editing in a population of cells, comprising the steps of: a) contacting the population of cells in vitro with a first lipid nanoparticle (LNP) composition comprising a first genome editing tool and a second LNP composition comprising a second genome editing tool; and b) culturing the population of cells in vitro, wherein at least 70%, 80%, 90%, or 95% of the cells in the population of cells are viable 24 hours after the last contact with an LNP composition; thereby editing the population of cells. In some embodiments, at least 70% of the cells in the population of cells are viable 24 hours after the last contact with an LNP composition. In some embodiments, at least 80% of the cells in the population of cells are viable 24 hours after the last contact with an LNP composition. In some embodiments, at least 90% of the cells in the population of cells are viable 24 hours after the last contact with an LNP composition. In some embodiments, at least 95% of the cells in the population of cells are viable 24 hours after the last contact with an LNP composition. In some embodiments, the first genome editing tool comprises a guide RNA. In some embodiments, the method further comprising contacting the cell in vitro with a third LNP composition comprising a genome editing tool, and wherein at least two LNP compositions comprise a gRNA. In some embodiments, at least one LNP composition comprises an RNA-guided DNA binding agent. In some embodiments, the RNA-guided DNA binding agent is Cas9. In some embodiments, the method further comprises contacting the cell with a donor nucleic acid for insertion in a target sequence. In some embodiments, the second genome editing tool is an RNA-guided DNA binding agent. In some embodiments the RNA-guided DNA binding agent is an S. pyogenes Cas9.

In some embodiments, methods are provided for gene editing in a cell, comprising the steps of: a) contacting the cell in vitro with at least six lipid nucleic acid assembly compositions, wherein at least two to four of the lipid nucleic acid assembly compositions each comprise a guide RNA (gRNA), and wherein at least one lipid nucleic acid assembly composition comprises a first genome editing tool; b) expanding the cell in vitro; thereby editing the cell. In some embodiments, the first genome editing tool comprises a guide RNA. In some embodiments, the methods further comprise contacting the cell in vitro with a third lipid nucleic acid assembly composition comprising a genome editing tool, and wherein at least two lipid nucleic acid assembly compositions comprise a gRNA. In some embodiments, at least one lipid nucleic acid assembly composition comprises an RNA-guided DNA binding agent. In some embodiments, the RNA-guided DNA binding agent is a Cas9. In some embodiments, the methods further comprise contacting the cell with a donor nucleic acid. In some embodiments, the second genome editing tool is a Cas9. In some embodiments, the cell is a T cell. In some embodiments, the cell is a non-activated cell. In some embodiments, the cell is an activated cell. In some embodiments, the cell of (a) is activated after contact with at least one lipid nucleic acid assembly composition.

In some embodiments, the cell is contacted with 1, 2, 3, 4, 5, 6, 7, 8, 9, 10, or 11 lipid nucleic acid assembly compositions. In some embodiments, this results in a cell having 1, 2, 3, 4, 5, 6, 7, 8, 9, 10, 11, 12, or more genome edits, e.g., based on differing gRNAs.

In some embodiments, the cell is contacted with one or more lipid nucleic acid assembly compositions having one or more genome editing tools in a single lipid nucleic acid assembly composition. In some embodiments, the single lipid nucleic acid assembly composition comprises multiple guide RNAs. In some embodiments, the single lipid nucleic acid assembly composition comprises 2-8, 2-6, 2-5, 2-4, 3-5, or 3-6 guide RNAs. In some embodiments, the single lipid nucleic acid assembly composition comprises 3-5 or 3-6 guide RNAs. In certain embodiments, the lipid nucleic acid assembly composition comprising more than one guide RNA further comprises an RNA guided-DNA binding agent. In certain embodiments, the lipid nucleic acid assembly composition comprising more than one guide RNA does not comprise an RNA guided-DNA binding agent.

In some embodiments, the contact between the cell and lipid nucleic acid assembly composition is sequential (one following another). In some embodiments, the contact between the cell and lipid nucleic acid assembly composition is simultaneous (contacts are concurrent or nearly concurrent). In some embodiments, the multiple lipid nucleic acid assembly compositions are administered sequentially. In some embodiments, the lipid nucleic acid assembly compositions are administered simultaneously. In some embodiments, the lipid nucleic acid assembly compositions are administered sequentially and simultaneously. In some embodiments, three lipid nucleic acid compositions are provided and two lipid nucleic acid compositions are administered first simultaneously, the cell is cultured for some period of time, and then the third lipid nucleic acid composition is administered (i.e., sequentially, after the administration of the first two composition). In another embodiment, three lipid nucleic acid compositions are provided and one lipid nucleic acid composition is administered first, the cell is cultured for some period of time, and then two lipid nucleic acid composition are administered simultaneously (and sequentially, after the administration of the first composition). Thus, simultaneous and sequential administration of lipid nucleic acid assembly composition may overlap in certain embodiments. In some embodiments, the first and second lipid nucleic acid assembly compositions each comprise a gRNA directed to a target sequence and optionally each also comprise an RNA-guided DNA binding agent. In some embodiments, the first and second lipid nucleic acid assembly compositions each comprise a gRNA directed to a target sequence, and may additionally comprise an RNA-guided DNA binding agent. In other words, the RNA-guided DNA binding agent may be provided to the cell by means other than the gRNA-containing lipid nucleic acid assembly compositions in some embodiments. In some embodiments, a gRNA and RNA-guided DNA binding agent may be co-encapsulated in a lipid nucleic acid assembly composition. In some embodiments, a gRNA and RNA-guided DNA binding agent may be provided to the cell in separate lipid nucleic acid assembly compositions. In some embodiments, the lipid nucleic acid assembly comprising an RNA-guided DNA binding agent is administered at a first time, simultaneously with a guide RNA, either in the same lipid nucleic acid assembly or in a different lipid nucleic acid assembly; followed by sequential administration of a guide RNA without further administration of an RNA-guided DNA binding agent. In some embodiments, the lipid nucleic acid assembly comprising an RNA-guided DNA binding agent is administered at a first time, simultaneously with a guide RNA, either in the same lipid nucleic acid assembly or in a different lipid nucleic acid assembly; followed by sequential administration of a guide RNA with an additional an RNA-guided DNA binding agent, optionally wherein the second RNA-guided DNA binding agent is different from the first RNA-guided DNA binding agent.

In some embodiments, the cells are frozen between sequential contacting or editing steps.

In some embodiments, the lipid nucleic acid assembly composition is pretreated with a serum factor before contacting the cell. In some embodiments, the lipid nucleic acid assembly composition is pretreated with a human serum before contacting the cell. In some embodiments, the lipid nucleic acid assembly composition is pretreated with a serum replacement, e.g., a commercially available serum replacement, preferably wherein the serum replacement is appropriate for ex vivo use. In some embodiments, the lipid nucleic acid assembly composition is pretreated with ApoE before contacting the cell. In some embodiments, the lipid nucleic acid assembly composition is pretreated with a recombinant ApoE3 or ApoE4 before contacting the cell. In some embodiments, the cell is serum-starved prior to contact with the lipid nucleic acid assembly composition.

In some embodiments, the multiplex methods comprise preincubating a serum factor and the lipid nucleic acid assembly composition for about 30 seconds to overnight. In some embodiments, the preincubation step comprises preincubating a serum factor and the lipid nucleic acid assembly composition for about 1 minute to 1 hour. In some embodiments, it comprises preincubating for about 1-30 minutes. In other embodiments, it comprises preincubating for about 1-10 minutes. Still further embodiments comprise preincubating for about 5 minutes.

In some embodiments, the preincubating step occurs at about 4° C. In some embodiments, the preincubating step occurs at about 25° C. In some embodiments, the preincubating step occurs at about 37° C. The preincubating step may comprise a buffer such as sodium bicarbonate or HEPES.

In some embodiments, a lipid nucleic acid assembly composition is provided to a “non-activated” cell. A “non-activated” cell refers to a cell that has not been stimulated in vitro. In some embodiments, a “non-activated” T cell may have been stimulated in vivo (e.g., by antigen) while in the body, however said cell may be referred to as non-activated herein if said cell has not been stimulated in vitro in culture. An “activated” cell is also useful in the methods disclosed herein and can refer to a cell that has been stimulated in vitro. Agents for activating cells in vitro are provided herein and are known in the art, particularly for activation of T cells or B cells.

In some embodiments, a T cell is cultured in culture medium prior to contact with a lipid nucleic acid assembly composition. In some embodiments, the T cell is cultured with one or more proliferative cytokines, for example one or more or all of IL-2, IL-15 and IL-21, and/or one or more agents that provides activation through CD3 and/or CD28.

In some embodiments, the T cell is activated prior to contact with a lipid nucleic acid assembly composition, is activated in between contact with lipid nucleic acid assembly compositions, and/or is activated after contact with a lipid nucleic acid assembly composition.

In some embodiments, the cell is a T cell and the method further comprises an activation step between a first and a second contacting step. In some embodiments, a non-activated T cell is contacted with one, two, or three nucleic acid assembly compositions. In some embodiments, an activated T cell is contacted with one to 8 lipid nucleic acid assembly compositions, optionally 1 to 4 lipid nucleic acid assembly compositions. In some embodiments, the T cell is contacted with at least 6 lipid nucleic acid assembly compositions. In some embodiments, the T cell is contacted with no more than 12 lipid nucleic acid assembly compositions. In some embodiments, the T cell is contacted with 2-12 lipid nucleic acid assembly compositions. In some embodiments, the T cell is contacted with 2-8 lipid nucleic acid assembly compositions. In some embodiments, the T cell is contacted with 2-6 lipid nucleic acid assembly compositions. In some embodiments, the T cell is contacted with 3-8 lipid nucleic acid assembly compositions. In some embodiments, the T cell is contacted with 3-6 lipid nucleic acid assembly compositions. In some embodiments, the T cell is contacted with 4-6 lipid nucleic acid assembly compositions. In some embodiments, the T cell is contacted with 4-12 lipid nucleic acid assembly compositions. In some embodiments, the T cell is contacted with 4-8 lipid nucleic acid assembly compositions. In some embodiments, the T cell is contacted with 6-12 lipid nucleic acid assembly compositions. In some embodiments, the T cell is contacted with 3, 4, 5, or 6 lipid nucleic acid assembly compositions. In some embodiments, the T cell is contacted with no more than 8 lipid nucleic acid assembly compositions simultaneously. In some embodiments, the T cell is contacted with no more than 6 lipid nucleic acid assembly compositions simultaneously. In some embodiments, the activated T cell is contacted with at least 6 lipid nucleic acid assembly compositions. In some embodiments, the activated T cell is contacted with no more than 12 lipid nucleic acid assembly compositions. In some embodiments, the activated T cell is contacted with 2-12 lipid nucleic acid assembly compositions. In some embodiments, the activated T cell is contacted with 4-12 lipid nucleic acid assembly compositions. In some embodiments, the activated T cell is contacted with 4-8 lipid nucleic acid assembly compositions. In some embodiments, the activated T cell is contacted with no more than 8 lipid nucleic acid assembly compositions simultaneously. In some embodiments, the activated T cell is contacted with no more than 6 lipid nucleic acid assembly compositions simultaneously.

In some embodiments, the T cell is contacted with at least a first lipid nucleic acid assembly composition comprising a first nucleic acid genome editing tool targeting a first target sequence, activated, and the activated T cell is contacted with at least a second lipid nucleic acid assembly composition comprising a second nucleic acid genome editing tool targeting a second target sequence. The activated T cell can be further contacted with additional lipid nucleic acid assembly compositions. In some embodiments, the T cell is contacted with two lipid nucleic acid assembly compositions, activated, and the activated is contacted with a third lipid nucleic acid assembly compositions, and optionally the activated cell is contacted with additional lipid nucleic acid assembly compositions. In some embodiments, the T cell is contacted with three lipid nucleic acid assembly compositions, activated, and the activated is contacted with a third lipid nucleic acid assembly compositions, and optionally the activated cell is contacted with additional lipid nucleic acid assembly compositions. The activation step may improve the outcome of the multiple genome edits as compared to the same method without the activation step.

In some embodiments, methods are provided for producing multiple genome edits in an in vitro-cultured T cell, comprising the steps of: a) contacting the T cell in vitro with (i) a first lipid nucleic acid assembly composition comprising a guide RNA (gRNA) directed to a first target sequence and optionally (ii) one or two additional lipid nucleic acid assembly compositions, wherein each additional lipid nucleic acid assembly composition comprises a gRNA directed to a target sequence that differs from the first target sequence and/or a genome editing tool; b) activating the T cell in vitro; c) contacting the activated T cell in vitro with (i) a further nucleic acid assembly composition comprising a further guide RNA directed to a target sequence that differs from the target sequence(s) of (a) and optionally (ii) one or more lipid nucleic acid assembly compositions, wherein each lipid nucleic acid assembly composition comprises a guide RNA directed to a target sequence that differs from the target sequence(s) of (a) and from each other and/or a genome editing tool; d) expanding the cell in vitro; thereby producing multiple genome edits in the T cell. In some embodiments, the method comprises contacting the T cell with 2, 3, 4, 5, 6, 7, 8, 9, 10, or 11 lipid nucleic acid assembly compositions, optionally 4-12 or 4-8 lipid nucleic acid assembly compositions. In some embodiments, the method comprises contacting the cell or T cell with 4-12 or 4-8 lipid nucleic acid assembly compositions. In some embodiments, the T cell of step (a) is contacted with two lipid nucleic acid assembly compositions, wherein the lipid nucleic acid assembly compositions are administered sequentially or simultaneously. In some embodiments, the T cell of step (a) is contacted with three lipid nucleic acid assembly compositions, wherein the lipid nucleic acid assembly compositions are administered: (i) sequentially; (ii) simultaneously; or (iii) simultaneously (two compositions) and sequentially (one composition administered before or after). In some embodiments, the T cell of step (c) is contacted with one to 8 lipid nucleic acid assembly compositions, optionally 1 to 4 lipid nucleic acid assembly compositions, wherein the lipid nucleic acid assembly compositions are administered: (i) sequentially; (ii) simultaneously; or (iii) simultaneously (at least two compositions) and sequentially (at least one composition administered before or after).

In some embodiments, methods are provided for gene editing in a cell, comprising the steps of a) contacting the cell in vitro with a first lipid nucleic acid assembly composition comprising a first genome editing tool and a second lipid nucleic acid assembly composition comprising a second genome editing tool; and b) expanding the cell in vitro; thereby editing the cell, wherein one of the lipid nucleic acid assembly compositions comprises a gRNA targeting TRAC. In some embodiments, one of the lipid nucleic acid assembly compositions comprises a gRNA targeting TRBC. In some embodiments, one of the lipid nucleic acid assembly compositions comprises a gRNA targeting a gene that reduces or eliminates surface expression of MEW class I. In some embodiments, one of the lipid nucleic acid assembly compositions comprises a gRNA targeting B2M. In some embodiments, one of the lipid nucleic acid assembly compositions comprises a gRNA targeting HLA-A, optionally wherein the cell is homozygous for HLA-B and homozygous for HLA-C. In some embodiments, one of the lipid nucleic acid assembly compositions comprises a gRNA targeting a gene that reduces or eliminates surface expression of MEW class II. In some embodiments, one of the lipid nucleic acid assembly compositions comprises a gRNA targeting CIITA.

In some embodiments, methods are provided for gene editing in a cell, comprising the steps of a) contacting the cell in vitro with a first lipid nucleic acid assembly composition comprising a first genome editing tool and a second lipid nucleic acid assembly composition comprising a second genome editing tool; and b) expanding the cell in vitro; thereby editing the cell, wherein the first and second lipid nucleic acid compositions each comprise a gRNA selected from a) a gRNA targeting TRAC, b) a gRNA targeting TRBC, c) a gRNA targeting B2M or a gRNA targeting HLA-A, and d) a gRNA targeting CIITA. In some embodiments, a further lipid nucleic acid assembly composition comprises an RNA-guided DNA binding agent. In some embodiments, the RNA-guided DNA binding agent is Cas9. In some embodiments, a further lipid nucleic acid assembly composition comprises a donor nucleic acid.

In some embodiments, methods are provided for gene editing in a cell, comprising the steps of a) contacting the cell in vitro with a first lipid nucleic acid assembly composition comprising a first genome editing tool and a second lipid nucleic acid assembly composition comprising a second genome editing tool; and b) expanding the cell in vitro; thereby editing the cell, wherein one of the lipid nucleic acid assembly compositions comprises a gRNA targeting TRAC, and one of the lipid nucleic acid assembly compositions comprises a gRNA targeting TRBC. In some embodiments, a further lipid nucleic acid assembly composition comprises an RNA-guided DNA binding agent. In some embodiments, the RNA-guided DNA binding agent is Cas9. In some embodiments, a further lipid nucleic acid assembly composition comprises a donor nucleic acid.

In some embodiments, methods are provided for gene editing in a cell, comprising the steps of a) contacting the cell in vitro with a first lipid nucleic acid assembly composition comprising a first genome editing tool and a second lipid nucleic acid assembly composition comprising a second genome editing tool; and b) expanding the cell in vitro; thereby editing the cell, wherein one of the lipid nucleic acid assembly compositions comprises a gRNA targeting TRAC, one of the lipid nucleic acid assembly compositions comprises a gRNA targeting TRBC, and a further lipid nucleic acid assembly composition comprises a gRNA targeting B2M. In some embodiments, a further lipid nucleic acid assembly composition comprises an RNA-guided DNA binding agent. In some embodiments, the RNA-guided DNA binding agent is Cas9. In some embodiments, a further lipid nucleic acid assembly composition comprises a donor nucleic acid.

In some embodiments, methods are provided for gene editing in a cell, comprising the steps of a) contacting the cell in vitro with a first lipid nucleic acid assembly composition comprising a first genome editing tool and a second lipid nucleic acid assembly composition comprising a second genome editing tool; and b) expanding the cell in vitro; thereby editing the cell, wherein one of the lipid nucleic acid assembly compositions comprises a gRNA targeting TRAC, one of the lipid nucleic acid assembly compositions comprises a gRNA targeting TRBC, and a further lipid nucleic acid assembly composition comprises a gRNA targeting HLA-A, optionally wherein the cell is homozygous for HLA-B and homozygous for HLA-C. In some embodiments, a further lipid nucleic acid assembly composition comprises an RNA-guided DNA binding agent. In some embodiments, the RNA-guided DNA binding agent is Cas9. In some embodiments, a further lipid nucleic acid assembly composition comprises a donor nucleic acid.

In some embodiments, methods are provided for gene editing in a cell, comprising the steps of a) contacting the cell in vitro with a first lipid nucleic acid assembly composition comprising a first genome editing tool and a second lipid nucleic acid assembly composition comprising a second genome editing tool; and b) expanding the cell in vitro; thereby editing the cell, wherein one of the lipid nucleic acid assembly compositions comprises a gRNA targeting TRAC, one of the lipid nucleic acid assembly compositions comprises a gRNA targeting TRBC, a further lipid nucleic acid assembly composition comprises a gRNA targeting B2M, and a further lipid nucleic acid assembly composition comprises a gRNA targeting CIITA. In some embodiments, a further lipid nucleic acid assembly composition comprises an RNA-guided DNA binding agent. In some embodiments, the RNA-guided DNA binding agent is Cas9. In some embodiments, a further lipid nucleic acid assembly composition comprises a donor nucleic acid.

In some embodiments, methods are provided for gene editing in a cell, comprising the steps of a) contacting the cell in vitro with a first lipid nucleic acid assembly composition comprising a first genome editing tool and a second lipid nucleic acid assembly composition comprising a second genome editing tool; and b) expanding the cell in vitro; thereby editing the cell, wherein one of the lipid nucleic acid assembly compositions comprises a gRNA targeting TRAC, one of the lipid nucleic acid assembly compositions comprises a gRNA targeting TRBC, a further lipid nucleic acid assembly composition comprises a gRNA targeting HLA-A, optionally wherein the cell is homozygous for HLA-B and homozygous for HLA-C, and a further lipid nucleic acid assembly composition comprises a gRNA targeting CIITA. In some embodiments, a further lipid nucleic acid assembly composition comprises an RNA-guided DNA binding agent. In some embodiments, the RNA-guided DNA binding agent is Cas9. In some embodiments, a further lipid nucleic acid assembly composition comprises a donor nucleic acid.

In some embodiments, the T cell is activated by polyclonal activation (or “polyclonal stimulation”) (not antigen-specific stimulation). In some embodiments, the T cell is activated by CD3 stimulation (e.g., providing an anti-CD3 antibody). In some embodiments, the T cell is activated by CD3 and CD28 stimulation (e.g., providing an anti-CD3 antibody and an anti-CD28 antibody). In some embodiments, the T cell is activated using a ready-to-use reagent to activate the T cell (e.g., via CD3/CD28 stimulation). In some embodiments, the T cell is activated by via CD3/CD28 stimulation provided by beads. In some embodiments, the T cell is activated by via CD3/CD28 stimulation wherein one or more components is soluble and/or one or more components is bound to a solid surface (e.g., plate or bead). In some embodiments, the T cell is activated by an antigen-independent mitogen (e.g., a lectin, including e.g., concanavalin A (“ConA”), or PHA).

In some embodiments, one or more cytokines are used for activation of T cells. IL-2 is provided for T cell activation. In some embodiments, the cytokine(s) for activation of T cells is a cytokine that binds to the common gamma chain (γc) receptor. In some embodiments, IL-2 is provided for T cell activation. In some embodiments, IL-7 is provided for T cell activation. In some embodiments, IL-7 is provided to promote T cell survival. In some embodiments, IL-15 is provided for T cell activation. In some embodiments, IL-21 is provided for T cell activation. In some embodiments, a combination of cytokines is provided for T cell activation, including e.g., IL-2, IL-7, IL-15, and/or IL-21.

In some embodiments, the T cell is activated by exposing the cell to an antigen (antigen stimulation). A T cell is activated by antigen when the antigen is presented as a peptide in a major histocompatibility complex (“MHC”) molecule (peptide-MHC complex). A cognate antigen may be presented to the T cell by co-culturing the T cell with an antigen-presenting cell (feeder cell) and antigen. In some embodiments, the T cell is activated by co-culture with an antigen-presenting cell that has been pulsed with antigen. In some embodiments, the antigen-presenting cell has been pulsed with a peptide of the antigen.

In some embodiments, the T cell may be activated for 12 to 72 hours. In some embodiments, the T cell may be activated for 12 to 48 hours. In some embodiments, the T cell may be activated for 12 to 24 hours. In some embodiments, the T cell may be activated for 24 to 48 hours. In some embodiments, the T cell may be activated for 24 to 72 hours. In some embodiments, the T cell may be activated for 12 hours. In some embodiments, the T cell may be activated for 48 hours. In some embodiments, the T cell may be activated for 72 hours.

In some embodiments, the methods provided herein do not include a selection step. In some embodiments, a selection step is included, and optionally the selection step is a physical sorting step (e.g., FACS or MACS) or a biochemical selection step (e.g., suicide gene, drug resistant selection, or antibody-toxin conjugate selection).

The lipid nucleic acid assembly compositions disclosed herein may be used in multiplex genome editing methods in vitro. The methods overcome existing problems with such methods by reducing toxicities associated with the transfection process itself. The reduced toxicity of each transfection event allows for multiple transactions and thereby multiple genome edits per cell.

In some embodiments, the genome edit comprises any one or more of an insertion, deletion, or substitution of at least one nucleotide in a target sequence. In some embodiments, the genome edit comprises an insertion of 1, 2, 3, 4 or 5 or more nucleotides in a target sequence. In some embodiments, the genome edit comprises a deletion of 1, 2, 3, 4 or 5 or more nucleotides in a target sequence. In other embodiments, the genome edit comprises an insertion of 1, 2, 3, 4, 5, 6, 7, 8, 9, 10, 15, 20 or 25 or more nucleotides in a target sequence. In other embodiments, the genome edit comprises a deletion of 1, 2, 3, 4, 5, 6, 7, 8, 9, 10, 15, 20 or 25 or more nucleotides in a target sequence. In some embodiments, the genome edit comprises an indel, which is generally defined in the art as an insertion or deletion of less than 1000 base pairs (bp). In some embodiments, the genome edit comprises an indel which results in a frameshift mutation in a target sequence. In some embodiments, the genome edit comprises a substitution of 1, 2, 3, 4, 5, 6, 7, 8, 9, 10, 15, 20 or 25 or more nucleotides in a target sequence. In some embodiments, the genome edit comprises one or more of an insertion, deletion, or substitution of nucleotides resulting from the incorporation of a template nucleic acid. In some embodiments, the genome edit comprises an insertion of a donor nucleic acid in a target sequence. In some embodiments, the edit or modification is not transient.

In some embodiments, one or more donor nucleic acids are provided for insertion in a target sequence. In some embodiments, the target sequence for insertion is a safe harbor locus. A safe harbor locus is a site in the genome able to accommodate the integration of an exogenous sequence without causing adverse alterations in the host genome and are known in the art. In some embodiments, the target sequence for insertion is in the β-2 microglobulin (B2M) gene. In some embodiments, the target sequence for insertion is in the class II major histocompatibility complex transactivator (CIITA) gene. In some embodiments, the target sequence for insertion is in the TRAC gene. In some embodiments, the target sequence for insertion is in AAVS1.

### III. CELL POPULATIONS AND METHODS/USES

A. Cell Populations

In some embodiments, compositions are provided herein comprising a cell population comprising edited cells comprising multiple genome edits per cell. In some embodiments, a cell population comprising edited cells comprising multiple genome edits per cell is provided, wherein at least 50% of the cells in the cell population comprise at least two genome edits and wherein: (i) fewer than 1%, fewer than 0.5%, fewer than 0.2%, or fewer than 0.1% of the cells in the cell population have a target-to-target translocation; or (ii) and the cell population has less than 2 times the background level of reciprocal translocations, complex translocations, or off-target translocations. In some embodiments, at least 50% of the cells in the cell population comprise at least two genome edits and fewer than 1% of the cells in the cell population have a target-to-target translocation. In some embodiments, at least 50% of the cells in the cell population comprise at least two genome edits and fewer than 0.5% of the cells in the cell population have a target-to-target translocation. In some embodiments, at least 50% of the cells in the cell population comprise at least two genome edits and fewer than 0.2% of the cells in the cell population have a target-to-target translocation. In some embodiments, at least 50% of the cells in the cell population comprise at least two genome edits and fewer than 0.1% of the cells in the cell population have a target-to-target translocation. In some embodiments, at least 50% of the cells in the cell population comprise at least two genome edits and the cell population has less than 2 times the background level of reciprocal translocations, complex translocations, or off-target translocations. In some embodiments, the cell population is capable of expansion 20-fold, 30-fold, 40-fold, or 50-fold ex vivo within 14 days in culture after initiation of editing. In some embodiments, the cell population is capable of expansion 20-fold, 30-fold, 40-fold, 50-fold, 60-fold, 70-fold, 80-fold, 90-fold, or 100-fold ex vivo within 14 days in culture after initiation of editing. In some embodiments, the cell population is capable of expansion 20-fold ex vivo within 14 days in culture after initiation of editing. In some embodiments, the cell population is capable of expansion 30-fold ex vivo within 14 days in culture after initiation of editing. In some embodiments, the cell population is capable of expansion 40-fold ex vivo within 14 days in culture after initiation of editing. In some embodiments, the cell population is capable of expansion 50-fold ex vivo within 14 days in culture after initiation of editing. In some embodiments, the cell population is capable of expansion 60-fold ex vivo within 14 days in culture after initiation of editing. In some embodiments, the cell population is capable of expansion 70-fold ex vivo within 14 days in culture after initiation of editing. In some embodiments, the cell population is capable of expansion 80-fold ex vivo within 14 days in culture after initiation of editing. In some embodiments, the cell population is capable of expansion 90-fold ex vivo within 14 days in culture after initiation of editing. In some embodiments, the cell population is capable of expansion 100-fold ex vivo within 14 days in culture after initiation of editing. In some embodiments, at least one genome edit of the multiple genome edits is produced by a genome editing tool comprising an RNA-guided DNA binding agent, wherein the RNA-guided DNA binding agent is optionally a cleavase. In some embodiments, at least two genome edits of the multiple genome edits are produced by a genome editing tool comprising an RNA-guided DNA binding agent, wherein the RNA-guided DNA binding agent is optionally a cleavase. In some embodiments, the multiple genome edits comprise an insertion of a donor nucleic acid, wherein the insertion is optionally a targeted insertion.

In some embodiments, a cell population comprising edited cells comprising multiple genome edits per cell is provided, wherein at least 60% of the cells in the cell population comprise at least two genome edits and wherein: (i) fewer than 1%, fewer than 0.5%, fewer than 0.2%, or fewer than 0.1% of the cells in the cell population have a target-to-target translocation; or (ii) the cell population has less than 2 times the background level of reciprocal translocations, complex translocations, or off-target translocations. In some embodiments, at least 60% of the cells in the cell population comprise at least two genome edits and fewer than 1% of the cells in the cell population have a target-to-target translocation. In some embodiments, at least 60% of the cells in the cell population comprise at least two genome edits and fewer than 0.5% of the cells in the cell population have a target-to-target translocation. In some embodiments, at least 60% of the cells in the cell population comprise at least two genome edits and fewer than 0.2% of the cells in the cell population have a target-to-target translocation. In some embodiments, at least 60% of the cells in the cell population comprise at least two genome edits and fewer than 0.1% of the cells in the cell population have a target-to-target translocation. In some embodiments, at least 60% of the cells in the cell population comprise at least two genome edits and the cell population has less than 2 times the background level of reciprocal translocations, complex translocations, or off-target translocations. In some embodiments, the cell population is capable of expansion 20-fold, 30-fold, 40-fold, or 50-fold ex vivo within 14 days in culture after initiation of editing. In some embodiments, the cell population is capable of expansion 20-fold, 30-fold, 40-fold, 50-fold, 60-fold, 70-fold, 80-fold, 90-fold, or 100-fold ex vivo within 14 days in culture after initiation of editing. In some embodiments, the cell population is capable of expansion 20-fold ex vivo within 14 days in culture after initiation of editing. In some embodiments, the cell population is capable of expansion 30-fold ex vivo within 14 days in culture after initiation of editing. In some embodiments, the cell population is capable of expansion 40-fold ex vivo within 14 days in culture after initiation of editing. In some embodiments, the cell population is capable of expansion 50-fold ex vivo within 14 days in culture after initiation of editing. In some embodiments, the cell population is capable of expansion 60-fold ex vivo within 14 days in culture after initiation of editing. In some embodiments, the cell population is capable of expansion 70-fold ex vivo within 14 days in culture after initiation of editing. In some embodiments, the cell population is capable of expansion 80-fold ex vivo within 14 days in culture after initiation of editing. In some embodiments, the cell population is capable of expansion 90-fold ex vivo within 14 days in culture after initiation of editing. In some embodiments, the cell population is capable of expansion 100-fold ex vivo within 14 days in culture after initiation of editing. In some embodiments, at least one genome edit of the multiple genome edits is produced by a genome editing tool comprising an RNA-guided DNA binding agent, wherein the RNA-guided DNA binding agent is optionally a cleavase. In some embodiments, at least two genome edits of the multiple genome edits are produced by a genome editing tool comprising an RNA-guided DNA binding agent, wherein the RNA-guided DNA binding agent is optionally a cleavase. In some embodiments, the multiple genome edits comprise an insertion of a donor nucleic acid, wherein the insertion is optionally a targeted insertion.

In some embodiments, a cell population comprising edited cells comprising multiple genome edits per cell is provided, wherein at least 70% of the cells in the cell population comprise at least two genome edits and wherein: (i) fewer than 1%, fewer than 0.5%, fewer than 0.2%, or fewer than 0.1% of the cells in the cell population have a target-to-target translocation; or (ii) the cell population has less than 2 times the background level of reciprocal translocations, complex translocations, or off-target translocations. In some embodiments, at least 70% of the cells in the cell population comprise at least two genome edits and fewer than 1% of the cells in the cell population have a target-to-target translocation. In some embodiments, at least 70% of the cells in the cell population comprise at least two genome edits and fewer than 0.5% of the cells in the cell population have a target-to-target translocation. In some embodiments, at least 70% of the cells in the cell population comprise at least two genome edits and fewer than 0.2% of the cells in the cell population have a target-to-target translocation. In some embodiments, at least 70% of the cells in the cell population comprise at least two genome edits and fewer than 0.1% of the cells in the cell population have a target-to-target translocation. In some embodiments, at least 70% of the cells in the cell population comprise at least two genome edits and the cell population has less than 2 times the background level of reciprocal translocations, complex translocations, or off-target translocations. In some embodiments, the cell population is capable of expansion 20-fold, 30-fold, 40-fold, or 50-fold ex vivo within 14 days in culture after initiation of editing. In some embodiments, the cell population is capable of expansion 20-fold, 30-fold, 40-fold, 50-fold, 60-fold, 70-fold, 80-fold, 90-fold, or 100-fold ex vivo within 14 days in culture after initiation of editing. In some embodiments, the cell population is capable of expansion 20-fold ex vivo within 14 days in culture after initiation of editing. In some embodiments, the cell population is capable of expansion 30-fold ex vivo within 14 days in culture after initiation of editing. In some embodiments, the cell population is capable of expansion 40-fold ex vivo within 14 days in culture after initiation of editing. In some embodiments, the cell population is capable of expansion 50-fold ex vivo within 14 days in culture after initiation of editing. In some embodiments, the cell population is capable of expansion 60-fold ex vivo within 14 days in culture after initiation of editing. In some embodiments, the cell population is capable of expansion 70-fold ex vivo within 14 days in culture after initiation of editing. In some embodiments, the cell population is capable of expansion 80-fold ex vivo within 14 days in culture after initiation of editing. In some embodiments, the cell population is capable of expansion 90-fold ex vivo within 14 days in culture after initiation of editing. In some embodiments, the cell population is capable of expansion 100-fold ex vivo within 14 days in culture after initiation of editing. In some embodiments, at least one genome edit of the multiple genome edits is produced by a genome editing tool comprising an RNA-guided DNA binding agent, wherein the RNA-guided DNA binding agent is optionally a cleavase. In some embodiments, at least two genome edits of the multiple genome edits are produced by a genome editing tool comprising an RNA-guided DNA binding agent, wherein the RNA-guided DNA binding agent is optionally a cleavase. In some embodiments, the multiple genome edits comprise an insertion of a donor nucleic acid, wherein the insertion is optionally a targeted insertion.

In some embodiments, a cell population comprising edited cells comprising multiple genome edits per cell is provided, wherein at least 80% of the cells in the cell population comprise at least two genome edits and wherein: (i) fewer than 1%, fewer than 0.5%, fewer than 0.2%, or fewer than 0.1% of the cells in the cell population have a target-to-target translocation; or (ii) the cell population has less than 2 times the background level of reciprocal translocations, complex translocations, or off-target translocations. In some embodiments, at least 80% of the cells in the cell population comprise at least two genome edits and fewer than 1% of the cells in the cell population have a target-to-target translocation. In some embodiments, at least 80% of the cells in the cell population comprise at least two genome edits and fewer than 0.5% of the cells in the cell population have a target-to-target translocation. In some embodiments, at least 80% of the cells in the cell population comprise at least two genome edits and fewer than 0.2% of the cells in the cell population have a target-to-target translocation. In some embodiments, at least 80% of the cells in the cell population comprise at least two genome edits and fewer than 0.1% of the cells in the cell population have a target-to-target translocation. In some embodiments, at least 80% of the cells in the cell population comprise at least two genome edits and the cell population has less than 2 times the background level of reciprocal translocations, complex translocations, or off-target translocations. In some embodiments, the cell population is capable of expansion 20-fold, 30-fold, 40-fold, or 50-fold ex vivo within 14 days in culture after initiation of editing. In some embodiments, the cell population is capable of expansion 20-fold, 30-fold, 40-fold, 50-fold, 60-fold, 70-fold, 80-fold, 90-fold, or 100-fold ex vivo within 14 days in culture after initiation of editing. In some embodiments, the cell population is capable of expansion 20-fold ex vivo within 14 days in culture after initiation of editing. In some embodiments, the cell population is capable of expansion 30-fold ex vivo within 14 days in culture after initiation of editing. In some embodiments, the cell population is capable of expansion 40-fold ex vivo within 14 days in culture after initiation of editing. In some embodiments, the cell population is capable of expansion 50-fold ex vivo within 14 days in culture after initiation of editing. In some embodiments, the cell population is capable of expansion 60-fold ex vivo within 14 days in culture after initiation of editing. In some embodiments, the cell population is capable of expansion 70-fold ex vivo within 14 days in culture after initiation of editing. In some embodiments, the cell population is capable of expansion 80-fold ex vivo within 14 days in culture after initiation of editing. In some embodiments, the cell population is capable of expansion 90-fold ex vivo within 14 days in culture after initiation of editing. In some embodiments, the cell population is capable of expansion 100-fold ex vivo within 14 days in culture after initiation of editing. In some embodiments, at least one genome edit of the multiple genome edits is produced by a genome editing tool comprising an RNA-guided DNA binding agent, wherein the RNA-guided DNA binding agent is optionally a cleavase. In some embodiments, at least two genome edits of the multiple genome edits are produced by a genome editing tool comprising an RNA-guided DNA binding agent, wherein the RNA-guided DNA binding agent is optionally a cleavase. In some embodiments, the multiple genome edits comprise an insertion of a donor nucleic acid, wherein the insertion is optionally a targeted insertion.

In some embodiments, a cell population comprising edited cells comprising multiple genome edits per cell is provided, wherein at least 90% of the cells in the cell population comprise at least two genome edits and wherein: (i) fewer than 1%, fewer than 0.5%, fewer than 0.2%, or fewer than 0.1% of the cells in the cell population have a target-to-target translocation; or (ii) the cell population has less than 2 times the background level of reciprocal translocations, complex translocations, or off-target translocations. In some embodiments, at least 90% of the cells in the cell population comprise at least two genome edits and fewer than 1% of the cells in the cell population have a target-to-target translocation. In some embodiments, at least 90% of the cells in the cell population comprise at least two genome edits and fewer than 0.5% of the cells in the cell population have a target-to-target translocation. In some embodiments, at least 90% of the cells in the cell population comprise at least two genome edits and fewer than 0.2% of the cells in the cell population have a target-to-target translocation. In some embodiments, at least 90% of the cells in the cell population comprise at least two genome edits and fewer than 0.1% of the cells in the cell population have a target-to-target translocation. In some embodiments, at least 90% of the cells in the cell population comprise at least two genome edits and the cell population has less than 2 times the background level of reciprocal translocations, complex translocations, or off-target translocations. In some embodiments, the cell population is capable of expansion 20-fold, 30-fold, 40-fold, or 50-fold ex vivo within 14 days in culture after initiation of editing. In some embodiments, the cell population is capable of expansion 20-fold, 30-fold, 40-fold, 50-fold, 60-fold, 70-fold, 80-fold, 90-fold, or 100-fold ex vivo within 14 days in culture after initiation of editing. In some embodiments, the cell population is capable of expansion 20-fold ex vivo within 14 days in culture after initiation of editing. In some embodiments, the cell population is capable of expansion 30-fold ex vivo within 14 days in culture after initiation of editing. In some embodiments, the cell population is capable of expansion 40-fold ex vivo within 14 days in culture after initiation of editing. In some embodiments, the cell population is capable of expansion 50-fold ex vivo within 14 days in culture after initiation of editing. In some embodiments, the cell population is capable of expansion 60-fold ex vivo within 14 days in culture after initiation of editing. In some embodiments, the cell population is capable of expansion 70-fold ex vivo within 14 days in culture after initiation of editing. In some embodiments, the cell population is capable of expansion 80-fold ex vivo within 14 days in culture after initiation of editing. In some embodiments, the cell population is capable of expansion 90-fold ex vivo within 14 days in culture after initiation of editing. In some embodiments, the cell population is capable of expansion 100-fold ex vivo within 14 days in culture after initiation of editing. In some embodiments, at least one genome edit of the multiple genome edits is produced by a genome editing tool comprising an RNA-guided DNA binding agent, wherein the RNA-guided DNA binding agent is optionally a cleavase. In some embodiments, at least two genome edits of the multiple genome edits are produced by a genome editing tool comprising an RNA-guided DNA binding agent, wherein the RNA-guided DNA binding agent is optionally a cleavase. In some embodiments, the multiple genome edits comprise an insertion of a donor nucleic acid, wherein the insertion is optionally a targeted insertion.

In some embodiments, a cell population comprising edited cells comprising multiple genome edits per cell is provided, wherein at least 95% of the cells in the cell population comprise at least two genome edits and wherein: (i) fewer than 1%, fewer than 0.5%, fewer than 0.2%, or fewer than 0.1% of the cells in the cell population have a target-to-target translocation; or (ii) the cell population has less than 2 times the background level of reciprocal translocations, complex translocations, or off-target translocations. In some embodiments, at least 95% of the cells in the cell population comprise at least two genome edits and fewer than 1% of the cells in the cell population have a target-to-target translocation. In some embodiments, at least 95% of the cells in the cell population comprise at least two genome edits and fewer than 0.5% of the cells in the cell population have a target-to-target translocation. In some embodiments, at least 95% of the cells in the cell population comprise at least two genome edits and fewer than 0.2% of the cells in the cell population have a target-to-target translocation. In some embodiments, at least 95% of the cells in the cell population comprise at least two genome edits and fewer than 0.1% of the cells in the cell population have a target-to-target translocation. In some embodiments, at least 95% of the cells in the cell population comprise at least two genome edits and the cell population has less than 2 times the background level of reciprocal translocations, complex translocations, or off-target translocations. In some embodiments, the cell population is capable of expansion 20-fold, 30-fold, 40-fold, or 50-fold ex vivo within 14 days in culture after initiation of editing. In some embodiments, the cell population is capable of expansion 20-fold, 30-fold, 40-fold, 50-fold, 60-fold, 70-fold, 80-fold, 90-fold, or 100-fold ex vivo within 14 days in culture after initiation of editing. In some embodiments, the cell population is capable of expansion 20-fold ex vivo within 14 days in culture after initiation of editing. In some embodiments, the cell population is capable of expansion 30-fold ex vivo within 14 days in culture after initiation of editing. In some embodiments, the cell population is capable of expansion 40-fold ex vivo within 14 days in culture after initiation of editing. In some embodiments, the cell population is capable of expansion 50-fold ex vivo within 14 days in culture after initiation of editing. In some embodiments, the cell population is capable of expansion 60-fold ex vivo within 14 days in culture after initiation of editing. In some embodiments, the cell population is capable of expansion 70-fold ex vivo within 14 days in culture after initiation of editing. In some embodiments, the cell population is capable of expansion 80-fold ex vivo within 14 days in culture after initiation of editing. In some embodiments, the cell population is capable of expansion 90-fold ex vivo within 14 days in culture after initiation of editing. In some embodiments, the cell population is capable of expansion 100-fold ex vivo within 14 days in culture after initiation of editing. In some embodiments, at least one genome edit of the multiple genome edits is produced by a genome editing tool comprising an RNA-guided DNA binding agent, wherein the RNA-guided DNA binding agent is optionally a cleavase. In some embodiments, at least two genome edits of the multiple genome edits are produced by a genome editing tool comprising an RNA-guided DNA binding agent, wherein the RNA-guided DNA binding agent is optionally a cleavase. In some embodiments, the multiple genome edits comprise an insertion of a donor nucleic acid, wherein the insertion is optionally a targeted insertion.

In some embodiments, a cell population comprising edited cells comprising multiple genome edits per cell is provided, wherein at least 50% of the cells in the cell population comprise at least two genome edits and wherein the cell population is capable of expansion 50-fold ex vivo within 14 days in culture after initiation of editing. In some embodiments, the cell population is capable of expansion 20-fold, 30-fold, 40-fold, or 50-fold ex vivo within 14 days in culture after initiation of editing. In some embodiments, the cell population is capable of expansion 20-fold, 30-fold, 40-fold, 50-fold, 60-fold, 70-fold, 80-fold, 90-fold, or 100-fold ex vivo within 14 days in culture after initiation of editing. In some embodiments, at least 50% of the cells in the cell population comprise at least two genome edits and wherein the cell population is capable of expansion 20-fold ex vivo within 14 days in culture after initiation of editing. In some embodiments, at least 50% of the cells in the cell population comprise at least two genome edits and wherein the cell population is capable of expansion 30-fold ex vivo within 14 days in culture after initiation of editing. In some embodiments, at least 50% of the cells in the cell population comprise at least two genome edits and wherein the cell population is capable of expansion 40-fold ex vivo within 14 days in culture after initiation of editing. In some embodiments, at least 50% of the cells in the cell population comprise at least two genome edits and wherein the cell population is capable of expansion 60-fold ex vivo within 14 days in culture after initiation of editing. In some embodiments, at least 50% of the cells in the cell population comprise at least two genome edits and wherein the cell population is capable of expansion 70-fold ex vivo within 14 days in culture after initiation of editing. In some embodiments, at least 50% of the cells in the cell population comprise at least two genome edits and wherein the cell population is capable of expansion 80-fold ex vivo within 14 days in culture after initiation of editing. In some embodiments, at least 50% of the cells in the cell population comprise at least two genome edits and wherein the cell population is capable of expansion 90-fold ex vivo within 14 days in culture after initiation of editing. In some embodiments, at least 50% of the cells in the cell population comprise at least two genome edits and wherein the cell population is capable of expansion 100-fold ex vivo within 14 days in culture after initiation of editing. In some embodiments, fewer than 1% of the cells in the cell population have a target-to-target translocation. In some embodiments, fewer than 0.5% of the cells in the cell population have a target-to-target translocation. In some embodiments, fewer than 0.2% of the cells in the cell population have a target-to-target translocation. In some embodiments, fewer than 0.1% of the cells in the cell population have a target-to-target translocation. In some embodiments, the cell population has less than 2 times the background level of reciprocal translocations, complex translocations, or off-target translocations. In some embodiments, at least one genome edit of the multiple genome edits is produced by a genome editing tool comprising an RNA-guided DNA binding agent, wherein the RNA-guided DNA binding agent is optionally a cleavase. In some embodiments, at least two genome edits of the multiple genome edits are produced by a genome editing tool comprising an RNA-guided DNA binding agent, wherein the RNA-guided DNA binding agent is optionally a cleavase. In some embodiments, the multiple genome edits comprise an insertion of a donor nucleic acid, wherein the insertion is optionally a targeted insertion.

In some embodiments, a cell population comprising edited cells comprising multiple genome edits per cell is provided, wherein at least 60% of the cells in the cell population comprise at least two genome edits and wherein the cell population is capable of expansion 50-fold ex vivo within 14 days in culture after initiation of editing. In some embodiments, the cell population is capable of expansion 20-fold, 30-fold, 40-fold, or 50-fold ex vivo within 14 days in culture after initiation of editing. In some embodiments, the cell population is capable of expansion 20-fold, 30-fold, 40-fold, 50-fold, 60-fold, 70-fold, 80-fold, 90-fold, or 100-fold ex vivo within 14 days in culture after initiation of editing. In some embodiments, at least 60% of the cells in the cell population comprise at least two genome edits and wherein the cell population is capable of expansion 20-fold ex vivo within 14 days in culture after initiation of editing. In some embodiments, at least 60% of the cells in the cell population comprise at least two genome edits and wherein the cell population is capable of expansion 30-fold ex vivo within 14 days in culture after initiation of editing. In some embodiments, at least 60% of the cells in the cell population comprise at least two genome edits and wherein the cell population is capable of expansion 40-fold ex vivo within 14 days in culture after initiation of editing. In some embodiments, at least 60% of the cells in the cell population comprise at least two genome edits and wherein the cell population is capable of expansion 60-fold ex vivo within 14 days in culture after initiation of editing. In some embodiments, at least 60% of the cells in the cell population comprise at least two genome edits and wherein the cell population is capable of expansion 70-fold ex vivo within 14 days in culture after initiation of editing. In some embodiments, at least 60% of the cells in the cell population comprise at least two genome edits and wherein the cell population is capable of expansion 80-fold ex vivo within 14 days in culture after initiation of editing. In some embodiments, at least 60% of the cells in the cell population comprise at least two genome edits and wherein the cell population is capable of expansion 90-fold ex vivo within 14 days in culture after initiation of editing. In some embodiments, at least 60% of the cells in the cell population comprise at least two genome edits and wherein the cell population is capable of expansion 100-fold ex vivo within 14 days in culture after initiation of editing. In some embodiments, fewer than 1% of the cells in the cell population have a target-to-target translocation. In some embodiments, fewer than 0.5% of the cells in the cell population have a target-to-target translocation. In some embodiments, fewer than 0.2% of the cells in the cell population have a target-to-target translocation. In some embodiments, fewer than 0.1% of the cells in the cell population have a target-to-target translocation. In some embodiments, the cell population has less than 2 times the background level of reciprocal translocations, complex translocations, or off-target translocations. In some embodiments, at least one genome edit of the multiple genome edits is produced by a genome editing tool comprising an RNA-guided DNA binding agent, wherein the RNA-guided DNA binding agent is optionally a cleavase. In some embodiments, at least two genome edits of the multiple genome edits are produced by a genome editing tool comprising an RNA-guided DNA binding agent, wherein the RNA-guided DNA binding agent is optionally a cleavase. In some embodiments, the multiple genome edits comprise an insertion of a donor nucleic acid, wherein the insertion is optionally a targeted insertion.

In some embodiments, a cell population comprising edited cells comprising multiple genome edits per cell is provided, wherein at least 70% of the cells in the cell population comprise at least two genome edits and wherein the cell population is capable of expansion 50-fold ex vivo within 14 days in culture after initiation of editing In some embodiments, the cell population is capable of expansion 20-fold, 30-fold, 40-fold, or 50-fold ex vivo within 14 days in culture after initiation of editing. In some embodiments, the cell population is capable of expansion 20-fold, 30-fold, 40-fold, 50-fold, 60-fold, 70-fold, 80-fold, 90-fold, or 100-fold ex vivo within 14 days in culture after initiation of editing. In some embodiments, at least 70% of the cells in the cell population comprise at least two genome edits and wherein the cell population is capable of expansion 20-fold ex vivo within 14 days in culture after initiation of editing. In some embodiments, at least 70% of the cells in the cell population comprise at least two genome edits and wherein the cell population is capable of expansion 30-fold ex vivo within 14 days in culture after initiation of editing. In some embodiments, at least 70% of the cells in the cell population comprise at least two genome edits and wherein the cell population is capable of expansion 40-fold ex vivo within 14 days in culture after initiation of editing. In some embodiments, at least 70% of the cells in the cell population comprise at least two genome edits and wherein the cell population is capable of expansion 60-fold ex vivo within 14 days in culture after initiation of editing. In some embodiments, at least 70% of the cells in the cell population comprise at least two genome edits and wherein the cell population is capable of expansion 70-fold ex vivo within 14 days in culture after initiation of editing. In some embodiments, at least 70% of the cells in the cell population comprise at least two genome edits and wherein the cell population is capable of expansion 80-fold ex vivo within 14 days in culture after initiation of editing. In some embodiments, at least 70% of the cells in the cell population comprise at least two genome edits and wherein the cell population is capable of expansion 90-fold ex vivo within 14 days in culture after initiation of editing. In some embodiments, at least 70% of the cells in the cell population comprise at least two genome edits and wherein the cell population is capable of expansion 100-fold ex vivo within 14 days in culture after initiation of editing. In some embodiments, fewer than 1% of the cells in the cell population have a target-to-target translocation. In some embodiments, fewer than 0.5% of the cells in the cell population have a target-to-target translocation. In some embodiments, fewer than 0.2% of the cells in the cell population have a target-to-target translocation. In some embodiments, fewer than 0.1% of the cells in the cell population have a target-to-target translocation. In some embodiments, the cell population has less than 2 times the background level of reciprocal translocations, complex translocations, or off-target translocations. In some embodiments, at least one genome edit of the multiple genome edits is produced by a genome editing tool comprising an RNA-guided DNA binding agent, wherein the RNA-guided DNA binding agent is optionally a cleavase. In some embodiments, at least two genome edits of the multiple genome edits are produced by a genome editing tool comprising an RNA-guided DNA binding agent, wherein the RNA-guided DNA binding agent is optionally a cleavase. In some embodiments, the multiple genome edits comprise an insertion of a donor nucleic acid, wherein the insertion is optionally a targeted insertion.

In some embodiments, a cell population comprising edited cells comprising multiple genome edits per cell is provided, wherein at least 80% of the cells in the cell population comprise at least two genome edits and wherein the cell population is capable of expansion 50-fold ex vivo within 14 days in culture after initiation of editing. In some embodiments, the cell population is capable of expansion 20-fold, 30-fold, 40-fold, or 50-fold ex vivo within 14 days in culture after initiation of editing. In some embodiments, the cell population is capable of expansion 20-fold, 30-fold, 40-fold, 50-fold, 60-fold, 70-fold, 80-fold, 90-fold, or 100-fold ex vivo within 14 days in culture after initiation of editing. In some embodiments, at least 80% of the cells in the cell population comprise at least two genome edits and wherein the cell population is capable of expansion 20-fold ex vivo within 14 days in culture after initiation of editing. In some embodiments, at least 80% of the cells in the cell population comprise at least two genome edits and wherein the cell population is capable of expansion 30-fold ex vivo within 14 days in culture after initiation of editing. In some embodiments, at least 80% of the cells in the cell population comprise at least two genome edits and wherein the cell population is capable of expansion 40-fold ex vivo within 14 days in culture after initiation of editing. In some embodiments, at least 80% of the cells in the cell population comprise at least two genome edits and wherein the cell population is capable of expansion 60-fold ex vivo within 14 days in culture after initiation of editing. In some embodiments, at least 80% of the cells in the cell population comprise at least two genome edits and wherein the cell population is capable of expansion 70-fold ex vivo within 14 days in culture after initiation of editing. In some embodiments, at least 80% of the cells in the cell population comprise at least two genome edits and wherein the cell population is capable of expansion 80-fold ex vivo within 14 days in culture after initiation of editing. In some embodiments, at least 80% of the cells in the cell population comprise at least two genome edits and wherein the cell population is capable of expansion 90-fold ex vivo within 14 days in culture after initiation of editing. In some embodiments, at least 80% of the cells in the cell population comprise at least two genome edits and wherein the cell population is capable of expansion 100-fold ex vivo within 14 days in culture after initiation of editing. In some embodiments, fewer than 1% of the cells in the cell population have a target-to-target translocation. In some embodiments, fewer than 0.5% of the cells in the cell population have a target-to-target translocation. In some embodiments, fewer than 0.2% of the cells in the cell population have a target-to-target translocation. In some embodiments, fewer than 0.1% of the cells in the cell population have a target-to-target translocation. In some embodiments, the cell population has less than 2 times the background level of reciprocal translocations, complex translocations, or off-target translocations. In some embodiments, at least one genome edit of the multiple genome edits is produced by a genome editing tool comprising an RNA-guided DNA binding agent, wherein the RNA-guided DNA binding agent is optionally a cleavase. In some embodiments, at least two genome edits of the multiple genome edits are produced by a genome editing tool comprising an RNA-guided DNA binding agent, wherein the RNA-guided DNA binding agent is optionally a cleavase. In some embodiments, the multiple genome edits comprise an insertion of a donor nucleic acid, wherein the insertion is optionally a targeted insertion.

In some embodiments, a cell population comprising edited cells comprising multiple genome edits per cell is provided, wherein at least 90% of the cells in the cell population comprise at least two genome edits and wherein the cell population is capable of expansion 50-fold ex vivo within 14 days in culture after initiation of editing. In some embodiments, the cell population is capable of expansion 20-fold, 30-fold, 40-fold, or 50-fold ex vivo within 14 days in culture after initiation of editing. In some embodiments, the cell population is capable of expansion 20-fold, 30-fold, 40-fold, 50-fold, 60-fold, 70-fold, 80-fold, 90-fold, or 100-fold ex vivo within 14 days in culture after initiation of editing. In some embodiments, at least 90% of the cells in the cell population comprise at least two genome edits and wherein the cell population is capable of expansion 20-fold ex vivo within 14 days in culture after initiation of editing. In some embodiments, at least 90% of the cells in the cell population comprise at least two genome edits and wherein the cell population is capable of expansion 30-fold ex vivo within 14 days in culture after initiation of editing. In some embodiments, at least 90% of the cells in the cell population comprise at least two genome edits and wherein the cell population is capable of expansion 40-fold ex vivo within 14 days in culture after initiation of editing. In some embodiments, at least 90% of the cells in the cell population comprise at least two genome edits and wherein the cell population is capable of expansion 60-fold ex vivo within 14 days in culture after initiation of editing. In some embodiments, at least 90% of the cells in the cell population comprise at least two genome edits and wherein the cell population is capable of expansion 70-fold ex vivo within 14 days in culture after initiation of editing. In some embodiments, at least 90% of the cells in the cell population comprise at least two genome edits and wherein the cell population is capable of expansion 80-fold ex vivo within 14 days in culture after initiation of editing. In some embodiments, at least 90% of the cells in the cell population comprise at least two genome edits and wherein the cell population is capable of expansion 90-fold ex vivo within 14 days in culture after initiation of editing. In some embodiments, at least 90% of the cells in the cell population comprise at least two genome edits and wherein the cell population is capable of expansion 100-fold ex vivo within 14 days in culture after initiation of editing. In some embodiments, fewer than 1% of the cells in the cell population have a target-to-target translocation. In some embodiments, fewer than 0.5% of the cells in the cell population have a target-to-target translocation. In some embodiments, fewer than 0.2% of the cells in the cell population have a target-to-target translocation. In some embodiments, fewer than 0.1% of the cells in the cell population have a target-to-target translocation. In some embodiments, the cell population has less than 2 times the background level of reciprocal translocations, complex translocations, or off-target translocations. In some embodiments, at least one genome edit of the multiple genome edits is produced by a genome editing tool comprising an RNA-guided DNA binding agent, wherein the RNA-guided DNA binding agent is optionally a cleavase. In some embodiments, at least two genome edits of the multiple genome edits are produced by a genome editing tool comprising an RNA-guided DNA binding agent, wherein the RNA-guided DNA binding agent is optionally a cleavase. In some embodiments, the multiple genome edits comprise an insertion of a donor nucleic acid, wherein the insertion is optionally a targeted insertion.

In some embodiments, a cell population comprising edited cells comprising multiple genome edits per cell is provided, wherein at least 95% of the cells in the cell population comprise at least two genome edits and wherein the cell population is capable of expansion 50-fold ex vivo within 14 days in culture after initiation of editing. In some embodiments, the cell population is capable of expansion 20-fold, 30-fold, 40-fold, or 50-fold ex vivo within 14 days in culture after initiation of editing. In some embodiments, the cell population is capable of expansion 20-fold, 30-fold, 40-fold, 50-fold, 60-fold, 70-fold, 80-fold, 90-fold, or 100-fold ex vivo within 14 days in culture after initiation of editing. In some embodiments, at least 95% of the cells in the cell population comprise at least two genome edits and wherein the cell population is capable of expansion 20-fold ex vivo within 14 days in culture after initiation of editing. In some embodiments, at least 95% of the cells in the cell population comprise at least two genome edits and wherein the cell population is capable of expansion 30-fold ex vivo within 14 days in culture after initiation of editing. In some embodiments, at least 95% of the cells in the cell population comprise at least two genome edits and wherein the cell population is capable of expansion 40-fold ex vivo within 14 days in culture after initiation of editing. In some embodiments, at least 95% of the cells in the cell population comprise at least two genome edits and wherein the cell population is capable of expansion 60-fold ex vivo within 14 days in culture after initiation of editing. In some embodiments, at least 95% of the cells in the cell population comprise at least two genome edits and wherein the cell population is capable of expansion 70-fold ex vivo within 14 days in culture after initiation of editing. In some embodiments, at least 95% of the cells in the cell population comprise at least two genome edits and wherein the cell population is capable of expansion 80-fold ex vivo within 14 days in culture after initiation of editing. In some embodiments, at least 95% of the cells in the cell population comprise at least two genome edits and wherein the cell population is capable of expansion 90-fold ex vivo within 14 days in culture after initiation of editing. In some embodiments, at least 95% of the cells in the cell population comprise at least two genome edits and wherein the cell population is capable of expansion 100-fold ex vivo within 14 days in culture after initiation of editing. In some embodiments, fewer than 1% of the cells in the cell population have a target-to-target translocation. In some embodiments, fewer than 0.5% of the cells in the cell population have a target-to-target translocation. In some embodiments, fewer than 0.2% of the cells in the cell population have a target-to-target translocation. In some embodiments, fewer than 0.1% of the cells in the cell population have a target-to-target translocation. In some embodiments, the cell population has less than 2 times the background level of reciprocal translocations, complex translocations, or off-target translocations. In some embodiments, at least one genome edit of the multiple genome edits is produced by a genome editing tool comprising an RNA-guided DNA binding agent, wherein the RNA-guided DNA binding agent is optionally a cleavase. In some embodiments, at least two genome edits of the multiple genome edits are produced by a genome editing tool comprising an RNA-guided DNA binding agent, wherein the RNA-guided DNA binding agent is optionally a cleavase. In some embodiments, the multiple genome edits comprise an insertion of a donor nucleic acid, wherein the insertion is optionally a targeted insertion.

As used herein, the “days in culture” if a cell has been frozen before culture, before editing, or between editing steps, the days in culture measurement starts from the day the cell is thawed and placed into culture. That is, the days in culture may be discontinuous.

As used herein, “after initiation of editing” refers to the time from when the cell or population of cells is contacted with a first LNP composition.

Target-to-target translocations, as described herein, may be detected using standard ddPCR assays.

In some embodiments, the cells of the cell population comprising edited cells are human cells. In some embodiments, the cells of the cell population comprising edited cells are selected from: mesenchymal stem cells; hematopoietic stem cells (HSCs); mononuclear cells; endothelial progenitor cells (EPCs); neural stem cells (NSCs); limbal stem cells (LSCs); tissue-specific primary cells or cells derived therefrom (TSCs), induced pluripotent stem cells (iPSCs); ocular stem cells; pluripotent stem cells (PSCs); embryonic stem cells (ESCs); cells for organ or tissue transplantations, and cells for use in ACT therapy.

In some embodiments, the cells of the cell population comprising edited cells are immune cells. In some embodiments, the cells of the cell population comprising edited cells are immune cells selected from lymphocytes (e.g., T cell, B cell, natural killer cell (“NK cell”, and NKT cell, or iNKT cell)), monocytes, macrophages, mast cells, dendritic cells, granulocytes (e.g., neutrophil, eosinophil, and basophil), primary immune cells, CD3+ cells, CD4+ cells, CD8+ T cells, regulatory T cells (Tregs), B cells, NK cells, and dendritic cells (DC)). In some embodiments, the cells of the cell population comprising edited cells are immune cells selected from peripheral blood mononuclear cell (PBMC), a lymphocyte, a T cell, optionally a CD4+ cell, a CD8+ cell, a memory T cell, a naïve T cell, a stem-cell memory T cell; or a B cell, optionally a memory B cell, a naïve B cell; and a primary cell. In some embodiments, the cells of the cell population comprising edited cells are T cells. In some embodiments, the cells of the cell population comprising edited cells are T cells selected from tumor infiltrating lymphocytes (TILs), T cells expressing an alpha-beta TCR, T cells expressing a gamma-delta TCR, a regulatory T cells (Treg), memory T cells, and early stem cell memory T cells (Tscm, CD27+/CD45+).

In some embodiments, the cells of the cell population comprising edited cells are immune cells isolated from human donor PBMCs or leukopacs before editing. In some embodiments, the cells of the cell population comprising edited cells are immune cells derived from a progenitor cell.

In some embodiments, the cells of the cell population comprising edited cells are non-activated immune cells. In some embodiments, the cells of the cell population comprising edited cells are activated immune cells.

In some embodiments, the cells of the cell population comprising edited cells comprising multiple genome edits comprise a third genome edit.

In some embodiments, the cells of the cell population comprising edited cells are for transfer into a human subject.

In some embodiments, at least 95% of the cells in the cell population comprise a genome edit of an endogenous TCR sequence. In some embodiments, at least 96% of the cells in the cell population comprise a genome edit of an endogenous TCR sequence. In some embodiments, at least 97% of the cells in the cell population comprise a genome edit of an endogenous TCR sequence. In some embodiments, at least 98% of the cells in the cell population comprise a genome edit of an endogenous TCR sequence. In some embodiments, at least 99% of the cells in the cell population comprise a genome edit of an endogenous TCR sequence.

In some embodiments, the cell population comprises edited cells with a genome edit comprising an insertion of an exogenous nucleic acid sequence coding for a targeting ligand or an alternative antigen binding moiety wherein at least 70% of the cells of the cell population comprise an insertion of an exogenous nucleic acid into a target sequence. In some embodiments, the cell population comprises edited cells with a genome edit comprising an insertion of an exogenous nucleic acid sequence coding for a targeting ligand or an alternative antigen binding moiety wherein at least 80% of the cells of the cell population comprise an insertion of an exogenous nucleic acid into a target sequence. In some embodiments, the cell population comprises edited cells with a genome edit comprising an insertion of an exogenous nucleic acid coding for a targeting ligand or an alternative antigen binding moiety wherein at least 90% of the cells of the cell population comprise an insertion of an exogenous nucleic acid into a target sequence. In some embodiments, the cell population comprises edited cells with a genome edit comprising an insertion of an exogenous nucleic acid coding for a targeting ligand or an alternative antigen binding moiety wherein at least 95% of the cells of the cell population comprise an insertion of an exogenous nucleic acid into a target sequence.

In some embodiments, the cell population comprises edited T cells, wherein at least 30%, 40%, 50%, 55%, 60%, or 65% of the cells of the cell population have a memory phenotype (CD27+, CD45RA+). In some embodiments, the cell population comprises edited T cells, wherein at least 30% of the cells of the cell population have a memory phenotype (CD27+, CD45RA+). In some embodiments, the cell population comprises edited T cells, wherein at least 40% of the cells of the cell population have a memory phenotype (CD27+, CD45RA+). In some embodiments, the cell population comprises edited T cells, wherein at least 50% of the cells of the cell population have a memory phenotype (CD27+, CD45RA+). In some embodiments, the cell population comprises edited T cells, wherein at least 55% of the cells of the cell population have a memory phenotype (CD27+, CD45RA+). In some embodiments, the cell population comprises edited T cells, wherein at least 60% of the cells of the cell population have a memory phenotype (CD27+, CD45RA+). In some embodiments, the cell population comprises edited T cells, wherein at least 65% of the cells of the cell population have a memory phenotype (CD27+, CD45RA+).

In some embodiments, the cell population comprising edited cells comprises cells with reduced or eliminated surface expression of MEW class I and/or MEW class II. In some embodiments, the cell population comprising edited cells comprises cells with reduced or eliminated surface expression of both MEW class I and MEW class II. In some embodiments, the cell population comprising edited cells comprises cells with reduced or eliminated surface expression HLA-A and the cells are homozygous for HLA-B and homozygous for HLA-C.

In some embodiments, the cell population comprising edited T cells comprises cells with reduced or eliminated surface expression of MEW class I and/or MEW class II. In some embodiments, the cell population comprising edited T cells comprises cells with reduced or eliminated surface expression of both MEW class I and MEW class II. In some embodiments, the cell population comprising edited T cells comprises cells with reduced or eliminated surface expression HLA-A and the cells are homozygous for HLA-B and homozygous for HLA-C.

In some embodiments, a population of cells is produced according to the provided multiplex delivery and genome editing methods. In some embodiments, at least 50% or more of the cells in the population comprises more than one genome edit. In some embodiments, at least 50%, 55%, 60%, 65%, 70%, 75%, 80%, 85%, 90%, 91%, 92%, 93%, 94%, 95%, 96%, 97%, 98%, 99%, or 100% (i.e., all cells as determined by the method of detection) of the cells in the population comprises more than one genome edit. In some embodiments, a method disclosed herein results in at least 5%, at least 10%, at least 15%, at least 20%, at least 25%, preferably at least 30%, at least 35%, at least 40%, at least 45%, at least 50%, at least 55%, at least 60%, at least 65%, at least 70%, at least 75%, at least 80%, at least 85%, at least 90% or at least 95% of the cells having at least two genome edits. In other embodiments, a method disclosed herein, results in at least 5%, at least 10%, at least 15%, at least 20%, at least 25%, preferably at least 30%, at least 35%, at least 40%, at least 45%, at least 50%, at least 55%, at least 60%, at least 65%, at least 70%, at least 75%, at least 80%, at least 85%, at least 90% or at least 95% of the cells having 2, 3, 4, 5, 6, 7, or 8 genome edits. In some embodiments, a method disclosed herein results in about 5% to about 100%, about 10% to about 50%, about 20 to about 100%, about 20 to about 80%, about 40 to about 100%, or about 40 to about 80% of every cell in a population having at least two genome edits. In some embodiments, the cells have not undergone a selection process, e.g., FACS or a biochemical selection process, at the completion of editing to enrich the population for edited cells.

In some embodiments, the delivery methods and genome editing methods produce expanded cells in vitro with increased survival. In embodiments, the improved survival rate is may be compared to cells treated with electroporation processes. In embodiments, the cell survival rate of an expanded cell is at least 70%, 80%, 90%, or 95%.

In some embodiments, the delivery methods and genome editing methods produced cells in vitro with low toxicity. For example, in embodiments, the resultant cells of the disclosed methods have less than 2%, 1%, 0.5%, 0.2%, 0.1% translocations, including e.g., target-target translocations, and/or off-target translocations. In some embodiments, the resultant cells of the disclosed method have less than 1%, 0.5%, 0.2%, 0.1% target-target translocations. In some embodiments, the resultant cells of the disclosed methods no measurable translocations, including e.g., target-target translocations, and/or off-target translocations. In some embodiments, the resultant cells have no measurable reciprocal translocations as determined, for example, using the methods provided herein. In some embodiments, the resultant cells have no measurable complex translocations as determined, for example, using the methods provided herein. In some embodiments, the resultant cells have no measurable off-target translocations as determined, for example, using the methods provided herein. In some embodiments, the resultant cells have less than 2 times the background level of reciprocal translocations, complex translocations, or off-target translocations, as determined, for example, using the methods provided herein.

In some embodiments, the genome editing methods produce cells with high editing efficiency. A particular advantage of the disclosed methods are the high editing rates observed in cells having multiple genome edits. For example, in some embodiments, the percent editing efficiency is at least 60%, 70%, 80%, 90%, or 95% at each target site.

It is understood that the number of cells in a population needed for any particular use depends, for example, on the type of cell and the intended use of the cell. The number of cells to be edited also depends on the ability to proliferate the cells after editing. It is also understood that the level of editing required, or the level of knockdown required, depends, at least in part, on the particular edit being made and the intended use of the cell population. For example, a population of B cells with genome editing, e.g., of 30% or less, 40% or less, 50% or less, may be useful in a protein expression system. For example, higher levels of knockdown are required of endogenous T cell receptor (TCR) on the surface of a T cell for transplantation into a subject, as low levels of endogenous TCR on the surface of the T cell can result in a severe adverse reaction when transplanted into a subject. Therefore, T cells expressing an endogenous TCR should be present in as low levels as possible in a population of T cells for transplantation purposes. However, editing of a T cell to produce a cytokine or other secreted factor, even for use in transplantation, may not require as high levels of editing as would be required for the endogenous TCR in a population of T cells for transplantation.

Exemplary edited cell population sizes are provided below. It is understood that the number of edited cells required for any particular indication may vary, e.g., therapeutic methods, may vary. Also, larger numbers of cells may be desirable for cell populations for use in allogenic therapies than for autologous therapies.

In certain embodiments, the population of cells comprising edited cells is a population of T cells. In certain embodiments the population of T cells comprises 1×10e9 edited T cells with multiple, i.e., at least 2, edits. In certain embodiments the population of T cells comprises 5×10e9 edited T cells with at least a single edit. In certain embodiments, the population of T cells comprises 1-10×10e9 edited T cells and is useful for TCR-T cell therapy. In certain embodiments, the population of T cells comprises 1×10e8 edited T cells and is useful for CAR-T therapy.

In certain embodiments, the population of cells comprising edited cells is a population of B cells. In certain embodiments, the population of B cells comprises 1-5×10e8 edited B cells with at least a single edit, preferably comprising edited B cells with multiple edits.

In certain embodiments, the population of cells comprising edited cells is a population of NK cells. In certain embodiments, the population of NK cells comprises 3×10e9 NK edited NK cells with at least a single edit. In certain embodiments, the population of NK cells comprises at least 5×10e8 edited NK cells with multiple edits. In certain embodiments, the population of NK cells comprises 1×10e8 to 9×10e9 edited NK cells for use in therapy.

In certain embodiments, the population of cells comprising edited cells is a population of monocytes or macrophages. In certain embodiments, the population of monocytes or macrophages comprising edited cells comprises at least 1×10e9 monocytes or macrophages having at least a single edit, or at least 2×10e8 monocytes or macrophages with multiple edits.

In certain embodiments, the population of cells comprising edited cells are dendritic cells. In certain embodiments, the population of dendritic cells comprises 5×10e6 to 5×10e7 edited dendritic cells.

In some embodiments, the genome editing methods to T cells in vitro have produced high editing efficiency at multiple target sites. In some embodiments, an engineered T cell is produced wherein the endogenous TCR is knocked out. In some embodiments, an engineered T cell is produced wherein expression of the endogenous TCR is reduced. In some embodiments, an engineered T cell is produced wherein three genes have reduced expression and/or are knocked out. In some embodiments, an engineered T cell is produced wherein four genes have reduced expression and/or are knocked out. In some embodiments, an engineered T cell is produced wherein five genes have reduced expression and/or are knocked out. In some embodiments, an engineered T cell is produced wherein six genes have reduced expression and/or are knocked out. In some embodiments, an engineered T cell is produced wherein seven genes have reduced expression and/or are knocked out. In some embodiments, an engineered T cell is produced wherein eight genes have reduced expression and/or are knocked out. In some embodiments, an engineered T cell is produced wherein nine genes have reduced expression and/or are knocked out. In some embodiments, an engineered T cell is produced wherein ten genes have reduced expression and/or are knocked out. In some embodiments, an engineered T cell is produced wherein eleven genes have reduced expression and/or are knocked out.

In some embodiments, an engineered T cell is produced wherein the endogenous TCR is knocked out and a transgenic TCR is inserted and expressed. In some embodiments, the engineered T cell is a primary human T cell. In some embodiments, the tgTCR targets Wilms' Tumor 1 (WT1). In some embodiments, the WT1 tgTCR is inserted into a high proportion of T cells (e.g., greater than 55%, 60%, 65%, 70%, 75%, 80%, 85%, 90%, or 95%) using the disclosed lipid nucleic acid assembly composition.

In some embodiments, the T cells produced by the disclosed methods have increased production of cytokines. In some embodiments, the increase in production of cytokines may be compared to T cells treated with electroporation processes. For example, in some embodiments, the genetically engineered T cells produced increased levels of IL-2. In some embodiments, the genetically engineered T cells produced increased levels of IFNγ. In some embodiments, the genetically engineered T cells produced increased levels of TNFα. Cytokine levels may be determined by standard methods, including e.g., ELISA, intracellular flow cytometry staining.

In some embodiments, the T cells produced by the disclosed methods demonstrate continued proliferation with repeat stimulation. For example, the T cells may proliferate following repeat stimulation in in vitro culture with an agent used to stimulate a T cell. In some embodiments, the T cell may be stimulated and proliferate in response to repeat stimulation with the cognate antigen for the T cell's TCR (e.g., peptide-WIC complexes on a cell that is co-cultured with the T cell). In some embodiments, the T cell may be stimulated and proliferate in response to repeat polyclonal stimulation. In some embodiments, the repeat stimulation is at least twice, three times, four times, five times, or more. In some embodiments, a proliferating the cell is expanded to form a population of cells that comprise the genetic modification.

In some embodiments, the T cells produced by the disclosed methods demonstrate increased expansion. In some embodiments, the increase in expansion may be compared to T cells treated with electroporation processes. Expansion may be evaluated by cell count, proliferation, or other standard methods for measuring expansion of T cells.

In some embodiments, the T cells produced by the disclosed methods exhibit a memory T cell phenotype. In some embodiments, the T cell memory phenotype referred to early stem-cell memory T cells (or “Tscm”) are particularly advantageous and are produced by the disclose methods. In some embodiments, a genetically engineered T cell has the Tscm phenotype (CD27+, CD45RA+).

In some embodiments, the engineered cell (e.g., T cell) produced by the disclosed method has reduced or eliminated surface expression of WIC class I and/or WIC class II. In some embodiments, the engineered cell has reduced or eliminated surface expression of both WIC class I and WIC class II. In some embodiments, the engineered cell has reduced or eliminated surface expression HLA-A and the cell is homozygous for HLA-B and homozygous for HLA-C.

In some embodiments, the engineered T cell produced by the disclosed methods has reduced or eliminated surface expression of MHC class I and/or MHC class II. In some embodiments, the engineered cell has reduced or eliminated surface expression of both MHC class I and MHC class II. In some embodiments, the engineered cell has reduced or eliminated surface expression HLA-A and the cell is homozygous for HLA-B and homozygous for HLA-C.

In some embodiments, one or more of all of the following advantages of the methods, reagents used therefore and products produced thereby are observed as compared to products produced by other methods of genome editing known in the art, e.g., electroporation:
a. improved ability to expand edited cells, e.g., 20-fold, 30-fold, 40-fold, or 50-fold expansion, optionally 60-fold, 70-fold, or 80-fold within 14 days in culture after initiation of editing;
b. comparable insertion rates with alternative methods such as electroporation;
c. reduced number/percentage of unedited cells, including increased percentage of cells having more than one edit, e.g., at least 2, 3, 4, 5, or 6 edits, i.e. due to greater editing efficiency, preferably without selection step to remove unedited cells or enrich for edited cells;
d. more desirable memory cell phenotype, e.g., at least 30%, 40%, preferably at least 50% having a memory T cell phenotype (CD27+, CD45RA+);
e. increased cytokine production (e.g., IL-2, IFNγ, TNFα), or other cytokines dependent on the cell type edited;
f. improved cytotoxicity of the edited cells;
g. improved proliferation and/or proliferative capacity of the edited cells;
h. enhanced durability of response with repeated stimulations, particularly in T cells; and/or
i. decreased rate of undesirable side effects and mutations, such as a decreased translocation rate, e.g., translocation rate of less than 2%, 1%, 0.5%, 0.2%, or 0.1% translocations, preferably target-to-target translocations; or less than twice the number of total translocations as compared to background.

B. Methods/Uses for Treating Disorders

The cell and/or population of cells provided herein produced by the disclosed multiplex methods may be used in methods of treating a variety of diseases and disorders.

In some embodiments, the disclosure provides a method of providing an immunotherapy in a subject, the method including administering to the subject an effective amount of a cell (e.g., a population of cells) as described herein, for example, a cell of any of the aforementioned cell aspects and embodiments.

In some embodiments of the methods, the method includes administering a lymphodepleting agent or immunosuppressant prior to administering to the subject an effective amount of the cell (e.g., a population of cells) as described herein, for example, a cell of any of the aforementioned cell aspects and embodiments. In another aspect, the disclosure provides a method of preparing cells (e.g., a population of cells).

Immunotherapy is the treatment of disease by activating or suppressing the immune system. Immunotherapies designed to elicit or amplify an immune response are classified as activation immunotherapies. Cell-based immunotherapies have been demonstrated to be effective in the treatment of some cancers. Immune effector cells such as lymphocytes, macrophages, dendritic cells, natural killer cells, cytotoxic T lymphocytes (CTLs) can be programmed to act in response to abnormal antigens expressed on the surface of tumor cells. Thus, cancer immunotherapy allows components of the immune system to destroy tumors or other cancerous cells. Cell-based immunotherapies have also been demonstrated to be effective in the treatment of autoimmune diseases or transplant rejection. Immune effector cells such as regulatory T cells (Tregs) or mesenchymal stem cells can be programmed to act in response to autoantigens or transplant antigens expressed on the surface of normal tissues.

In some embodiments, the disclosure provides a population of cells or a method of preparing cells (e.g., a population of cells). The population of cells may be used for immunotherapy.

Cells of the disclosure are suitable for further engineering, e.g., by introduction of further edited, or modified genes or alleles. In some embodiments, the polypeptide is a wild-type or variant TCR. Cells of the disclosure may also be suitable for further engineering by introduction of a heterologous sequence coding for an alternative antigen binding moiety, e.g., by introduction of a heterologous sequence coding for an alternative (non-endogenous) TCR, e.g., a chimeric antigen receptors (CAR) engineered to target a specific protein. CARs are also known as chimeric immunoreceptors, chimeric T cell receptors or artificial T cell receptors.

In some embodiments, the disclosure provides a method of treating a subject in need thereof that includes administering cells (e.g., a population of cells), e.g., cells prepared by a method of preparing cells described herein, for example, a method of any of the aforementioned aspects and embodiments of methods of preparing cells,

In some embodiments, the population of cells or cells produced by the disclosed methods can be used to treat cancer, infectious diseases, inflammatory diseases, autoimmune diseases, cardiovascular diseases, neurological diseases, ophthalmologic diseases, renal diseases, liver diseases, musculoskeletal diseases, red blood cell diseases, or transplant rejections.

In some embodiments, the cancer is lymphoma, breast cancer, lung cancer, multiple myeloma, leukemia, liver cancer, urinary tract cancer, kidney cancer, bladder cancer, melanoma, colorectal cancer, pancreatic cancer, epithelial malignancies, mesothelioma, oropharyngeal cancer, cervical cancer, uterine cancer, ovarian cancer, anogenital cancer, or brain cancer. In some embodiments, the lymphoma is non-Hodgkin's lymphoma, including diffuse large B cell lymphoma (DLBCL), aggressive B cell lymphoma, or high-grade B cell lymphoma, or mantle cell lymphoma. In some embodiments, the breast cancer is a triple negative breast cancer. In some embodiments, the lung cancer is non-small cell lung cancer (NSCLC) or small cell lung cancer (SCLC). In some embodiments, the leukemia is acute lymphoblastic leukemia or acute myeloid leukemia. In some embodiments, the cancer is a solid tumor.

In some embodiments, the infectious disease is caused by human immunodeficiency virus (HIV), Hepatitis A virus, Hepatitis C Virus, Hepatitis B Virus, Human Cytomegalovirus (CMV), Epstein-Barr virus, human papillomavirus, Mycobacterium tuberculosis, a human coronavirus, or invasive Aspergillus fumigatus. In some embodiments, the infectious disease is acquired immunodeficiency syndrome (AIDS), hepatitis A, hepatitis B, hepatitis C, tuberculosis, severe acute respiratory syndrome (SARS), middle east respiratory syndrome (MERS), or coronavirus disease 2019 (COVID-19). In some embodiments, the tuberculosis is multidrug-resistant (MDR) tuberculosis or extensively drug-resistant (XDR) tuberculosis. In some embodiments, the human coronavirus is middle east respiratory syndrome coronavirus (MERS-CoV), severe acute respiratory syndrome coronavirus (SARS-CoV), or severe acute respiratory syndrome coronavirus 2 (SARS-CoV2). In some embodiments, infectious disease is a human papillomavirus-positive cancer, such as uterine cancer, cervical cancer, or oropharyngeal cancer.

In some embodiments, the inflammatory disease is allergy, asthma, celiac disease, glomerulonephritis, inflammatory bowel disease, gout, rheumatoid arthritis (RA), myositis, scleroderma, ankylosing spondylitis (AS), antiphospholipid antibody syndrome (APS), systemic lupus erythematosus (SLE), Sjogren's syndrome, rheumatic heart disease, chronic obstructive pulmonary disease (COPD), or transplant rejection.

In some embodiments, the autoimmune disease is Type 1 diabetes, multiple sclerosis, Crohn's diseases, ulcerative colitis, autoimmune thyroid disease, rheumatoid arthritis (RA), inflammatory bowel disease, antiphospholipid antibody syndrome (APS), Sjogren's syndrome, scleroderma, psoriasis, psoriatic arthritis, Guillain-Barre syndrome, Addison's disease, Graves' disease, Hashimoto's thyroiditis, Myasthenia gravis, autoimmune vasculitis, autoimmune uveitis, autoimmune hepatitis, pernicious anemia, celiac disease, or systemic lupus erythematosus (SLE).

In some embodiments, the cardiovascular disease is ischemic heart disease, coronary heart disease, aorta disease, Marfan syndrome, congenital heart disease, heart valve disease, pericardial disease, rheumatic heart disease, peripheral arterial disease, or stroke.

In some embodiments, the neurological disease is Parkinson's disease, amyotrophic lateral sclerosis, stroke, spinal cord injury, Alzheimer's disease, age-related macular degeneration, traumatic brain injury, multiple sclerosis, Huntington's disease, muscular dystrophy, or Guillain-Barre syndrome.

In some embodiments, the ophthalmologic disease is glaucoma, retinopathy, macular degeneration, or cytomegalovirus (CMV) retinitis. In some embodiments, the ophthalmologic disease is a retinal disease. In some embodiments. The ophthalmologic disease is mediated by VEGF.

In some embodiments, the engineered cells produced by the disclosed methods can be used as a cell therapy comprising an autologous cell therapy. In some embodiments, the engineered cells can be used as a cell therapy comprising an allogeneic stem cell therapy. In some embodiments, the cell therapy comprises induced pluripotent stem cells (iPSCs). iPSCs may be induced to differentiate into other cell types including e.g., beta islet cells, neurons, and blood cells. In some embodiments, the cell therapy comprises hematopoietic stem cells. In some embodiments, the stem cells comprise mesenchymal stem cells that can develop into bone, cartilage, muscle, and fat cells. In some embodiments, the stem cells comprise ocular stem cells. In some embodiments, the allogeneic stem cell transplant comprises allogeneic bone marrow transplant. In some embodiments, the stem cells comprise pluripotent stem cells (PSCs). In some embodiments, the stem cells comprise induced embryonic stem cells (ESCs).

In some embodiments, the cell therapy is a transgenic T cell therapy. In some embodiments, the cell therapy comprises a Wilms' Tumor 1 (WT1) targeting transgenic T cell. In some embodiments, the cell therapy comprises a targeting receptor or a donor nucleic acid encoding a targeting receptor of a commercially available T cell therapy, such as a CAR T cell therapy. There are number of targeting receptors currently approved for cell therapy. The cells and methods provided herein can be used with these known constructs. Commercially approved cell products that include targeting receptor constructs for use as cell therapies include e.g., Kymriah® (tisagenlecleucel); Yescarta® (axicabtagene ciloleucel); Tecartus™ (brexucabtagene autoleucel); Tabelecleucel (Tab-cel®); Viralym-M (ALVR105); and Viralym-C.

C. Exemplary Cell Types

In some embodiments, the cell is an immune cell. As used herein, “immune cell” refers to a cell of the immune system, including e.g., a lymphocyte (e.g., T cell, B cell, natural killer cell (“NK cell”, and NKT cell, or iNKT cell)), monocyte, macrophage, mast cell, dendritic cell, or granulocyte (e.g., neutrophil, eosinophil, and basophil). In some embodiments, the cell is a primary immune cell. In some embodiments, the immune system cell may be selected from CD3⁺, CD4⁺ and CD8⁺ T cells, regulatory T cells (Tregs), B cells, NK cells, and dendritic cells (DC). In some embodiments, the immune cell is allogeneic.

In some embodiments, the cell is a lymphocyte. In some embodiments, the cell is an adaptive immune cell. In some embodiments, the cell is a T cell. In some embodiments, the cell is a B cell. In some embodiments, the cell is a NK cell.

As used herein, a T cell can be defined as a cell that expresses a T cell receptor (“TCR” or “αβ TCR” or “γδ TCR”), however in some embodiments, the TCR of a T cell may be genetically modified to reduce its expression (e.g., by genetic modification to the TRAC or TRBC genes), therefore expression of the protein CD3 may be used as a marker to identify a T cell by standard flow cytometry methods. CD3 is a multi-subunit signaling complex that associates with the TCR. Thus, a T cell may be referred to as CD3+. In some embodiments, a T cell is a cell that expresses a CD3+ marker and either a CD4+ or CD8+ marker.

In some embodiments, the T cell expresses the glycoprotein CD8 and therefore is CD8+ by standard flow cytometry methods and may be referred to as a “cytotoxic” T cell. In some embodiments, the T cell expresses the glycoprotein CD4 and therefore is CD4+ by standard flow cytometry methods and may be referred to as a “helper” T cell. CD4+ T cells can differentiate into subsets and may be referred to as a Th1 cell, Th2 cell, Th9 cell, Th17 cell, Th22 cell, T regulatory (“Treg”) cell, or T follicular helper cells (“Tfh”). Each CD4+ subset releases specific cytokines that can have either proinflammatory or anti-inflammatory functions, survival or protective functions. A T cell may be isolated from a subject by CD4+ or CD8+ selection methods.

In some embodiments, the T cell is a memory T cell. In the body, a memory T cell has encountered antigen. A memory T cell can be located in the secondary lymphoid organs (central memory T cells) or in recently infected tissue (effector memory T cells). A memory T cell may be a CD8+ T cell. A memory T cell may be a CD4+ T cell.

As used herein, a “central memory T cell” can be defined as an antigen-experienced T cell, and for example, may expresses CD62L and CD45RO. A central memory T cell may be detected as CD62L+ and CD45RO+ by Central memory T cells also express CCR7, therefore may be detected as CCR7+ by standard flow cytometry methods.

As used herein, an “early stem-cell memory T cell” (or “Tscm”) can be defined as a T cell that expresses CD27 and CD45RA, and therefore is CD27+ and CD45RA+ by standard flow cytometry methods. A Tscm does not express the CD45 isoform CD45RO, therefore a Tscm will further be CD45RO− if stained for this isoform by standard flow cytometry methods. A CD45RO− CD27+ cell is therefore also an early stem-cell memory T cell. Tscm cells further express CD62L and CCR7, therefore may be detected as CD62L+ and CCR7+ by standard flow cytometry methods. Early stem-cell memory T cells have been shown to correlate with increased persistence and therapeutic efficacy of cell therapy products.

In some embodiments, the cell is a B cell. As used herein, a “B cell” can be defined as a cell that expresses CD19 and/or CD20, and/or B cell mature antigen (“BCMA”), and therefore a B cell is CD19+, and/or CD20+, and/or BCMA+ by standard flow cytometry methods. A B cell is further negative for CD3 and CD56 by standard flow cytometry methods. The B cell may be a plasma cell. The B cell may be a memory B cell. The B cell may be a naïve B cell. The B cell may be IgM+ or has a class-switched B cell receptor (e.g., IgG+, or IgA+).

In some embodiments, the cell is a mononuclear cell, such as from bone marrow or peripheral blood. In some embodiments, the cell is a peripheral blood mononuclear cell (“PBMC”). In some embodiments, the cell is a PBMC, e.g. a lymphocyte or monocyte. In some embodiments, the cell is a peripheral blood lymphocyte (“PBL”).

Cells used in ACT therapy are included, such as mesenchymal stem cells (e.g., isolated from bone marrow (BM), peripheral blood (PB), placenta, umbilical cord (UC) or adipose); hematopoietic stem cells (HSCs; e.g. isolated from BM); mononuclear cells (e.g., isolated from BM or PB); endothelial progenitor cells (EPCs; isolated from BM, PB, and UC); neural stem cells (NSCs); limbal stem cells (LSCs); or tissue-specific primary cells or cells derived therefrom (TSCs). Cells used in ACT therapy further include induced pluripotent stem cells (iPSCs; see e.g., Mahla, International J. Cell Biol. 2016 (Article ID 6940283): 1-24 (2016)) that may be induced to differentiate into other cell types including e.g., islet cells, neurons, and blood cells; ocular stem cells; pluripotent stem cells (PSCs); embryonic stem cells (ESCs); cells for organ or tissue transplantations such as islet cells, cardiomyocytes, thyroid cells, thymocytes, neuronal cells, skin cells, retinal cells, chondrocytes, myocytes, and keratinocytes.

In some embodiments, the cell is a human cell, such as a cell from a subject. In some embodiments, the cell is isolated from a human subject. In some embodiments, the cell is isolated from a patient. In some embodiments, the cell is isolated from a donor. In some embodiments, the cell is isolated from human donor PBMCs or leukopaks. In some embodiments, the cell is from a subject with a condition, disorder, or disease. In some embodiments, the cell is from a human donor with Epstein Barr Virus (“EBV”).

In some embodiments, the cell is homozygous for HLA-B and homozygous for HLA-C. In some embodiments, the cell contains a genetic modification in the HLA-A gene and is homozygous for HLA-B and homozygous for HLA-C.

In some embodiments, the methods are carried out ex vivo. As used herein, “ex vivo” refers to an in vitro method wherein the cell is capable of being transferred into a subject, e.g. as an ACT therapy. In some embodiments, an ex vivo method is an in vitro method involving an ACT therapy cell or cell population.

In some embodiments, the cell is maintained in culture. In some embodiments, the cell is transplanted into a patient. In some embodiments, the cell is removed from a subject, genetically modified ex vivo, and then administered back to the same patient. In some embodiments, the cell is removed from a subject, genetically modified ex vivo, and then administered to a subject other than the subject from which it was removed.

In some embodiments, the cell is from a cell line. In some embodiments, the cell line is derived from a human subject. In some embodiments, the cell line is a lymphoblastoid cell line (“LCL”). The cell may be cryopreserved and thawed. The cell may not have been previously cryopreserved.

In some embodiments, the cell is from a cell bank. In some embodiments, the cell is genetically modified and then transferred into a cell bank. In some embodiments the cell is removed from a subject, genetically modified ex vivo, and transferred into a cell bank. In some embodiments, a genetically modified population of cells is transferred into a cell bank. In some embodiments, a genetically modified population of immune cells is transferred into a cell bank. In some embodiments, a genetically modified population of immune cells comprising a first and second subpopulations, wherein the first and second sub-populations have at least one common genetic modification and at least one different genetic modification are transferred into a cell bank.

### IV. EXEMPLARY GENOME EDITING TOOLS

In some embodiments, the lipid nucleic acid assembly comprises a genome editing tool or a nucleic acid encoding the same.

As used herein, the term “genome editing tool” (or “gene editing tool”) is any component of “genome editing system” (or “gene editing system”) necessary or helpful for producing an edit in the genome of a cell. In some embodiments, the present disclosure provides for methods of delivering genome editing tools of a genome editing system (for example a zinc finger nuclease system, a TALEN system, a meganuclease system or a CRISPR/Cas system) to a cell (or population of cells). Genome editing tools include, for example, nucleases capable of making single or double strand break in the DNA or RNA of a cell, e.g., in the genome of a cell. The genome editing tools, e.g. nucleases, may optionally modify the genome of a cell without cleaving the nucleic acid, or nickases. A genome editing nuclease or nickase may be encoded by an mRNA. Such nucleases include, for example, RNA-guided DNA binding agents, and CRISPR/Cas components. Genome editing tools include fusion proteins, including e.g., a nickase fused to an effector domain such as an editor domain. Genome editing tools include any item necessary or helpful for accomplishing the goal of a genome edit, such as, for example, guide RNA, sgRNA, dgRNA, donor nucleic acid, and the like.

Various suitable gene editing systems comprising genome editing tools for delivery with the lipid nucleic acid assembly compositions are described herein, including but not limited to the CRISPR/Cas system; zinc finger nuclease (ZFN) system; and the transcription activator-like effector nuclease (TALEN) system. Generally, the gene editing systems involve the use of engineered cleavage systems to induce a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA to guide specific cleavage or nicking of a target DNA sequence. Further, targeted nucleases are being developed based on the Argonaute system (e.g., from T. thermophilus, known as ‘TtAgo’, see Swarts et al (2014) Nature 507(7491): 258-261), which also may have the potential for uses in genome editing and gene therapy.

A. CRISPR/Cas Genome Editing Tools

In some embodiments, the genome editing tool is a component of a CRISPR/Cas system.

1. Guide RNA (gRNA)

In some embodiments, the genome editing tool is a guide RNA (gRNA), which can be a dual-guide RNA (dgRNA) or a single-guide RNA (sgRNA). A guide RNA directs an RNA-guided DNA binding agent to a target sequence.

In some embodiments of the present disclosure, the cargo for the lipid nucleic acid assembly formulation includes at least one gRNA or a nucleic acid encoding the same. The gRNA may guide the Cas nuclease or Class 2 Cas nuclease to a target sequence on a target nucleic acid molecule. In some embodiments, a gRNA binds with and provides specificity of cleavage by a Class 2 Cas nuclease. In some embodiments, the gRNA and the Cas nuclease may form a ribonucleoprotein (RNP), e.g., a CRISPR/Cas complex such as a CRISPR/Cas9 complex. In some embodiments, the CRISPR/Cas complex may be a Type-II CRISPR/Cas9 complex. In some embodiments, the CRISPR/Cas complex may be a Type-V CRISPR/Cas complex, such as a Cpf1/guide RNA complex. Cas nucleases and cognate gRNAs may be paired. The gRNA scaffold structures that pair with each Class 2 Cas nuclease vary with the specific CRISPR/Cas system.

In some embodiments, the sgRNA is a “Cas9 sgRNA” capable of mediating RNA-guided DNA cleavage by a Cas9 protein. In some embodiments, the sgRNA is a “Cpf1 sgRNA” capable of mediating RNA-guided DNA cleavage by a Cpf1 protein. In some embodiments, the gRNA comprises a crRNA and tracr RNA sufficient for forming an active complex with a Cas9 protein and mediating RNA-guided DNA cleavage. In some embodiments, the gRNA comprises a crRNA sufficient for forming an active complex with a Cpf1 protein and mediating RNA-guided DNA cleavage. See Zetsche 2015.

Certain embodiments of the disclosure also provide nucleic acids, e.g., expression cassettes, encoding the gRNA described herein. A “guide RNA nucleic acid” is used herein to refer to a guide RNA (e.g. an sgRNA or a dgRNA) and a guide RNA expression cassette, which is a nucleic acid that encodes one or more guide RNAs.

In some embodiments, the nucleic acid may be a DNA molecule. In some embodiments, the nucleic acid may comprise a nucleotide sequence encoding a crRNA. In some embodiments, the nucleotide sequence encoding the crRNA comprises a targeting sequence flanked by all or a portion of a repeat sequence from a naturally-occurring CRISPR/Cas system. In some embodiments, the nucleic acid may comprise a nucleotide sequence encoding a tracr RNA. In some embodiments, the crRNA and the tracr RNA may be encoded by two separate nucleic acids. In other embodiments, the crRNA and the tracr RNA may be encoded by a single nucleic acid. In some embodiments, the crRNA and the tracr RNA may be encoded by opposite strands of a single nucleic acid. In other embodiments, the crRNA and the tracr RNA may be encoded by the same strand of a single nucleic acid. In some embodiments, the gRNA nucleic acid encodes an sgRNA. In some embodiments, the gRNA nucleic acid encodes a Cas9 nuclease sgRNA. In come embodiments, the gRNA nucleic acid encodes a Cpf1 nuclease sgRNA.

The nucleotide sequence encoding the guide RNA may be operably linked to at least one transcriptional or regulatory control sequence, such as a promoter, a 3′ UTR, or a 5′ UTR. In one example, the promoter may be a tRNA promoter, e.g., tRNA^{Lys3}, or a tRNA chimera. See Mefferd et al., RNA. 2015 21:1683-9; Scherer et al., Nucleic Acids Res. 2007 35: 2620-2628. In some embodiments, the promoter may be recognized by RNA polymerase III (Pol III). Non-limiting examples of Pol III promoters also include U6 and H1 promoters. In some embodiments, the nucleotide sequence encoding the guide RNA may be operably linked to a mouse or human U6 promoter. In some embodiments, the gRNA nucleic acid is a modified nucleic acid. In some embodiments, the gRNA nucleic acid includes a modified nucleoside or nucleotide. In some embodiments, the gRNA nucleic acid includes a 5′ end modification, for example a modified nucleoside or nucleotide to stabilize and prevent integration of the nucleic acid. In some embodiments, the gRNA nucleic acid comprises a double-stranded DNA having a 5′ end modification on each strand. In some embodiments, the gRNA nucleic acid includes an inverted dideoxy-T or an inverted abasic nucleoside or nucleotide as the 5′ end modification. In some embodiments, the gRNA nucleic acid includes a label such as biotin, desthiobiotin-TEG, digoxigenin, and fluorescent markers, including, for example, FAM, ROX, TAMRA, and AlexaFluor.

In some embodiments, more than one gRNA nucleic acid, such as a gRNA, can be used with a CRISPR/Cas nuclease system. Each gRNA nucleic acid may contain a different targeting sequence, such that the CRISPR/Cas system cleaves more than one target sequence. In some embodiments, one or more gRNAs may have the same or differing properties such as activity or stability within a CRISPR/Cas complex. Where more than one gRNA is used, each gRNA can be encoded on the same or on different gRNA nucleic acid. The promoters used to drive expression of the more than one gRNA may be the same or different.

Target sequences for Cas proteins include both the positive and negative strands of genomic DNA (i.e., the sequence given and the sequence's reverse compliment), as a nucleic acid substrate for a Cas protein is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

The length of the targeting sequence may depend on the CRISPR/Cas system and components used. For example, different Class 2 Cas nucleases from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence length is 0, 1, 2, 3, 4, or 5 nucleotides longer or shorter than the guide sequence of a naturally-occurring CRISPR/Cas system. In some embodiments, the Cas nuclease and gRNA scaffold will be derived from the same CRISPR/Cas system. In some embodiments, the targeting sequence may comprise or consist of 18-24 nucleotides. In some embodiments, the targeting sequence may comprise or consist of 19-21 nucleotides. In some embodiments, the targeting sequence may comprise or consist of 20 nucleotides.

2. RNA-Guided DNA Binding Agent

In some embodiments, the genome editing tool is a RNA-guided DNA binding agent. In some embodiments, the RNA-guided DNA binding agent is a Cas cleavase/nickase and/or an inactivated forms thereof (dCas DNA binding agents). In some embodiments, the RNA-guided DNA binding agent is a Cas nuclease.

In some embodiments, the genome editing tool is an mRNA encoding an RNA-guided DNA binding agent. In some embodiments, the genome editing tool is an mRNA encoding a Cas nuclease.

In some embodiments, genome editing tool comprises a mRNA such as a Cas nuclease mRNA and a gRNA nucleic acid that are co-encapsulated in the lipid nucleic acid assembly composition. In some embodiments, an mRNA encoding a RNA-guided DNA binding agent is formulated in a first lipid nucleic acid assembly composition and a gRNA nucleic acid is formulated in a second lipid nucleic acid assembly composition. In some embodiments, the first and second lipid nucleic acid assembly compositions are administered simultaneously. In other embodiments, the first and second lipid nucleic acid assembly compositions are administered sequentially. In some embodiments, the first and second lipid nucleic acid assembly compositions are combined prior to the preincubation step. In some embodiments, the first and second lipid nucleic acid assembly compositions are preincubated separately.

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In some embodiments, the Cas nuclease is a Cpf1 nuclease from an Acidaminococcus or Lachnospiraceae.

Wild type Cas9 has two nuclease domains: RuvC and HNH. The RuvC domain cleaves the non-target DNA strand, and the HNH domain cleaves the target strand of DNA. In some embodiments, the Cas9 nuclease comprises more than one RuvC domain and/or more than one HNH domain. In some embodiments, the Cas9 nuclease is a wild type Cas9. In some embodiments, the Cas9 is capable of inducing a double strand break in target DNA. In some embodiments, the Cas nuclease may cleave dsDNA, it may cleave one strand of dsDNA, or it may not have DNA cleavase or nickase activity.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease or Cas nickase may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB—A0Q7Q2 (CPF1_FRATN)).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the RNA-guided DNA binding agent comprises a APOBEC3 deaminase. In some embodiments, a APOBEC3 deaminase is a APOBEC3A (A3A). In some embodiments, the A3A is a human A3A. In some embodiments, the A3A is a wild-type A3A.

In some embodiments, the RNA-guided DNA binding agent comprises an editor. An exemplary editor is BC22n which comprises a H. sapiens APOBEC3A fused to S. pyogenes-D10A Cas9 nickase by an XTEN linker.

In some embodiments, the RNA-guided DNA-binding agent comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be fused at the N-terminus or the C-terminus of the RNA-guided DNA-binding agent sequence. It may also be inserted within the RNA-guided DNA binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In some circumstances, the two NLSs may be the same (e.g., two SV40 NLSs) or different. In some embodiments, the RNA-guided DNA-binding agent is fused to two NLS sequences (e.g., SV40) fused at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 23) or PKKKRRV (SEQ ID NO: 24). In some embodiments, the NLS may be a bipartite sequence, such as the NLS of nucleoplasmin, KRPAATKKAGQAKKKK (SEQ ID NO: 25). In a specific embodiment, a single PKKKRKV (SEQ ID NO: 23) NLS may be fused at the C-terminus of the RNA-guided DNA-binding agent. One or more linkers are optionally included at the fusion site.

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub 1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBLS).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-binding agent to a specific organelle, cell type, tissue, or organ. In some embodiments, the heterologous functional domain may target the RNA-guided DNA-binding agent to mitochondria.

In further embodiments, the heterologous functional domain may be an effector domain such as an editor domain. When the RNA-guided DNA-binding agent is directed to its target sequence, e.g., when a Cas nuclease is directed to a target sequence by a gRNA, the effector domain such as an editor domain may modify or affect the target sequence. In some embodiments, the effector domain such as an editor domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA. In some embodiments, the DNA modification domain is a methylation domain, such as a demethylation or methyltransferase domain. In some embodiments, the effector domain is a DNA modification domain, such as a base-editing domain. In particular embodiments, the DNA modification domain is a nucleic acid editing domain that introduces a specific modification into the DNA, such as a deaminase domain. See, e.g., WO 2015/089406; US 2016/0304846. The nucleic acid editing domains, deaminase domains, and Cas9 variants described in WO 2015/089406 and U.S. 2016/0304846 are hereby incorporated by reference.

The nuclease may comprise at least one domain that interacts with a guide RNA (“gRNA”). Additionally, the nuclease may be directed to a target sequence by a gRNA. In Class 2 Cas nuclease systems, the gRNA interacts with the nuclease as well as the target sequence, such that it directs binding to the target sequence. In some embodiments, the gRNA provides the specificity for the targeted cleavage, and the nuclease may be universal and paired with different gRNAs to cleave different target sequences. Class 2 Cas nuclease may pair with a gRNA scaffold structure of the types, orthologs, and exemplary species listed above.

B. Additional Genome Editing System Tools

In some embodiments, the genome editing tool is a component of a genome editing system chosen from a zinc finger nuclease system, a TALEN system, and a meganuclease system. In some embodiments, the genome editing tool is a nucleic acid encoding one or more components of such genome editing system. Exemplary components of the system include meganucleases, zinc finger nucleases, TALENS, and fragments thereof.

In some embodiments, the gene editing system is a TALEN system. Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to a desired DNA sequence, to promote DNA cleavage at specific locations (see, e.g., Boch, 2011, Nature Biotech). The restriction enzymes can be introduced into cells, for use in gene editing or for genome editing in situ, a technique known as genome editing with engineered nucleases. Such methods and compositions for use therein are known in the art. See, e.g., WO2019147805, WO2014040370, WO2018073393, the contents of which are hereby incorporated in their entireties.

In some embodiments, the gene editing system is a zinc-finger system. Zinc-finger nucleases (ZFNs) are artificial restriction enzymes generated by fusing a zinc finger DNA-binding domain to a DNA-cleavage domain. Zinc finger domains can be engineered to target specific desired DNA sequences to enables zinc-finger nucleases to target unique sequences within complex genomes. The non-specific cleavage domain from the type IIs restriction endonuclease FokI is typically used as the cleavage domain in ZFNs. Cleavage is repaired by endogenous DNA repair machinery, allowing ZFN to precisely alter the genomes of higher organisms. Such methods and compositions for use therein are known in the art. See, e.g., WO2011091324, the contents of which are hereby incorporated in their entireties.

### V. EXEMPLARY NUCLEIC ACIDS FOR LIPID NUCLEIC ACID ASSEMBLY COMPOSITIONS

In some embodiments, the lipid nucleic acid assembly compositions deliver a nucleic acid (or polynucleotide) to a cell. In some embodiments, the nucleic acid comprises nucleosides or nucleoside analogs which have nitrogenous heterocyclic bases or base analogs linked together along a backbone, including conventional RNA, DNA, mixed RNA-DNA, and polymers that are analogs thereof.

A. Modified Nucleic Acids

In some embodiments, the lipid nucleic acid assembly compositions comprise modified RNAs. In some embodiments, the lipid nucleic acid assembly compositions comprise modified DNAs.

Modified nucleosides or nucleotides can be present in an RNA, for example a gRNA or mRNA. A gRNA or mRNA comprising one or more modified nucleosides or nucleotides, for example, is called a “modified” RNA to describe the presence of one or more non-naturally and/or naturally occurring components or configurations that are used instead of or in addition to the canonical A, G, C, and U residues. In some embodiments, a modified RNA is synthesized with a non-canonical nucleoside or nucleotide, here called “modified.”

Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification). Certain embodiments comprise a 5′ end modification to an mRNA, gRNA, or nucleic acid. Certain embodiments comprise a 3′ end modification to an mRNA, gRNA, or nucleic acid. A modified RNA can contain 5′ end and 3′ end modifications. A modified RNA can contain one or more modified residues at non-terminal locations. In some embodiments, a gRNA includes at least one modified residue. In some embodiments, an mRNA includes at least one modified residue.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

In some embodiments, a composition or formulation disclosed herein comprises an mRNA comprising an open reading frame (ORF), such as, e.g. an ORF encoding an RNA-guided DNA binding agent, such as a Cas nuclease, or Class 2 Cas nuclease as described herein. In some embodiments, an mRNA comprising an ORF encoding an RNA-guided DNA binding agent, such as a Cas nuclease or Class 2 Cas nuclease, is provided, used, or administered. In some embodiments, the ORF is codon optimized. In some embodiments, the ORF encoding an RNA-guided DNA binding agent is a “modified RNA-guided DNA binding agent ORF” or simply a “modified ORF,” which is used as shorthand to indicate that the ORF is modified in one or more of the following ways: (1) the modified ORF has a uridine content ranging from its minimum uridine content to 150% of the minimum uridine content; (2) the modified ORF has a uridine dinucleotide content ranging from its minimum uridine dinucleotide content to 150% of the minimum uridine dinucleotide content; (3) the modified ORF has at least 90% identity to any one of any of the Cas ORFs in Table 89; (4) the modified ORF consists of a set of codons of which at least 75% of the codons are minimal uridine codon(s) for a given amino acid, e.g. the codon(s) with the fewest uridines (usually 0 or 1 except for a codon for phenylalanine, where the minimal uridine codon has 2 uridines); or (5) the modified ORF comprises at least one modified uridine. In some embodiments, the modified ORF is modified in at least two, three, or four of the foregoing ways. In some embodiments, the modified ORF comprises at least one modified uridine and is modified in at least one, two, three, or all of (1)-(4) above.

“Modified uridine” is used herein to refer to a nucleoside other than thymidine with the same hydrogen bond acceptors as uridine and one or more structural differences from uridine. In some embodiments, a modified uridine is a substituted uridine, i.e., a uridine in which one or more non-proton substituents (e.g., alkoxy, such as methoxy) takes the place of a proton. In some embodiments, a modified uridine is pseudouridine. In some embodiments, a modified uridine is a substituted pseudouridine, i.e., a pseudouridine in which one or more non-proton substituents (e.g., alkyl, such as methyl) takes the place of a proton. In some embodiments, a modified uridine is any of a substituted uridine, pseudouridine, or a substituted pseudouridine.

“Uridine position” as used herein refers to a position in a polynucleotide occupied by a uridine or a modified uridine. Thus, for example, a polynucleotide in which “100% of the uridine positions are modified uridines” contains a modified uridine at every position that would be a uridine in a conventional RNA (where all bases are standard A, U, C, or G bases) of the same sequence. Unless otherwise indicated, a U in a polynucleotide sequence of a sequence table or sequence listing in, or accompanying, this disclosure can be a uridine or a modified uridine.

Minimal Uridine Codons:

| | Amino Acid | Minimal uridine codon |
|---|---|---|
| A | Alanine | GCA or GCC or GCG |
| G | Glycine | GGA or GGC or GGG |
| V | Valine | GUC or GUA or GUG |
| D | Aspartic acid | GAC |
| E | Glutamic acid | GAA or GAG |
| I | Isoleucine | AUC or AUA or AUG |
| T | Threonine | ACA or ACC or ACG |
| N | Asparagine | AAC |
| K | Lysine | AAG or AAA |
| S | Serine | AGC |
| R | Arginine | AGA or AGG |
| L | Leucine | CUG or CUA or CUC |
| P | Proline | CCG or CCA or CCC |
| H | Histidine | CAC or CAA or CAG |
| Q | Glutamine | CAG or CAA |
| F | Phenylalanine | UUC |
| Y | Tyrosine | UAC |
| C | Cysteine | UGC |
| W | Tryptophan | UGG |
| M | Methionine | AUG |

In any of the foregoing embodiments, the modified ORF may consist of a set of codons of which at least 75%, 80%, 85%, 90%, 95%, 98%, 99%, or 100% of the codons are codons listed in the Table above of minimal uridine codons. In any of the foregoing embodiments, the modified ORF may comprise a sequence with at least 90%, 95%, 98%, 99%, or 100% identity to any one of the Cas ORFs in Table 89.

In any of the foregoing embodiments, the modified ORF may have a uridine content ranging from its minimum uridine content to 150%, 145%, 140%, 135%, 130%, 125%, 120%, 115%, 110%, 105%, 104%, 103%, 102%, or 101% of the minimum uridine content.

In any of the foregoing embodiments, the modified ORF may have a uridine dinucleotide content ranging from its minimum uridine dinucleotide content to 150%, 145%, 140%, 135%, 130%, 125%, 120%, 115%, 110%, 105%, 104%, 103%, 102%, or 101% of the minimum uridine dinucleotide content.

In any of the foregoing embodiments, the modified ORF may comprise a modified uridine at least at one, a plurality of, or all uridine positions. In some embodiments, the modified uridine is a uridine modified at the 5 position, e.g., with a halogen, methyl, or ethyl. In some embodiments, the modified uridine is a pseudouridine modified at the 1 position, e.g., with a halogen, methyl, or ethyl. The modified uridine can be, for example, pseudouridine, N1-methyl-pseudouridine, 5-methoxyuridine, 5-iodouridine, or a combination thereof. In some embodiments, the modified uridine is 5-methoxyuridine. In some embodiments, the modified uridine is 5-iodouridine. In some embodiments, the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, at least 10%, 15%, 20%, 25%, 30%, 35%, 40%, 45%, 50%, 55%, 60%, 65%, 70%, 75%, 80%, 85%, 90%, 95%, 98%, 99%, or 100% of the uridine positions in an mRNA according to the disclosure are modified uridines. In some embodiments, 10%-25%, 15-25%, 25-35%, 35-45%, 45-55%, 55-65%, 65-75%, 75-85%, 85-95%, or 90-100% of the uridine positions in an mRNA according to the disclosure are modified uridines, e.g., 5-methoxyuridine, 5-iodouridine, N1-methyl pseudouridine, pseudouridine, or a combination thereof. In some embodiments, 10%-25%, 15-25%, 25-35%, 35-45%, 45-55%, 55-65%, 65-75%, 75-85%, 85-95%, or 90-100% of the uridine positions in an mRNA according to the disclosure are 5-methoxyuridine. In some embodiments, 10%-25%, 15-25%, 25-35%, 35-45%, 45-55%, 55-65%, 65-75%, 75-85%, 85-95%, or 90-100% of the uridine positions in an mRNA according to the disclosure are pseudouridine. In some embodiments, 10%-25%, 15-25%, 25-35%, 35-45%, 45-55%, 55-65%, 65-75%, 75-85%, 85-95%, or 90-100% of the uridine positions in an mRNA according to the disclosure are N1-methyl pseudouridine. In some embodiments, 10%-25%, 15-25%, 25-35%, 35-45%, 45-55%, 55-65%, 65-75%, 75-85%, 85-95%, or 90-100% of the uridine positions in an mRNA according to the disclosure are 5-iodouridine. In some embodiments, 10%-25%, 15-25%, 25-35%, 35-45%, 45-55%, 55-65%, 65-75%, 75-85%, 85-95%, or 90-100% of the uridine positions in an mRNA according to the disclosure are 5-methoxyuridine, and the remainder are N1-methyl pseudouridine. In some embodiments, 10%-25%, 15-25%, 25-35%, 35-45%, 45-55%, 55-65%, 65-75%, 75-85%, 85-95%, or 90-100% of the uridine positions in an mRNA according to the disclosure are 5-iodouridine, and the remainder are N1-methyl pseudouridine.

In any of the foregoing embodiments, the modified ORF may comprise a reduced uridine dinucleotide content, such as the lowest possible uridine dinucleotide (UU) content, e.g. an ORF that (a) uses a minimal uridine codon (as discussed above) at every position and (b) encodes the same amino acid sequence as the given ORF. The uridine dinucleotide (UU) content can be expressed in absolute terms as the enumeration of UU dinucleotides in an ORF or on a rate basis as the percentage of positions occupied by the uridines of uridine dinucleotides (for example, AUUAU would have a uridine dinucleotide content of 40% because 2 of 5 positions are occupied by the uridines of a uridine dinucleotide). Modified uridine residues are considered equivalent to uridines for the purpose of evaluating minimum uridine dinucleotide content.

In some embodiments, the mRNA comprises at least one UTR from an expressed mammalian mRNA, such as a constitutively expressed mRNA. An mRNA is considered constitutively expressed in a mammal if it is continually transcribed in at least one tissue of a healthy adult mammal. In some embodiments, the mRNA comprises a 5′ UTR, 3′ UTR, or 5′ and 3′ UTRs from an expressed mammalian RNA, such as a constitutively expressed mammalian mRNA. Actin mRNA is an example of a constitutively expressed mRNA.

In some embodiments, the mRNA comprises at least one UTR from Hydroxysteroid 17-Beta Dehydrogenase 4 (HSD17B4 or HSD), e.g., a 5′ UTR from HSD. In some embodiments, the mRNA comprises at least one UTR from a globin mRNA, for example, human alpha globin (HBA) mRNA, human beta globin (HBB) mRNA, or Xenopus laevis beta globin (XBG) mRNA. In some embodiments, the mRNA comprises a 5′ UTR, 3′ UTR, or 5′ and 3′ UTRs from a globin mRNA, such as HBA, HBB, or XBG. In some embodiments, the mRNA comprises a 5′ UTR from bovine growth hormone, cytomegalovirus (CMV), mouse Hba-a1, HSD, an albumin gene, HBA, HBB, or XBG. In some embodiments, the mRNA comprises a 3′ UTR from bovine growth hormone, cytomegalovirus, mouse Hba-a1, HSD, an albumin gene, HBA, HBB, or XBG. In some embodiments, the mRNA comprises 5′ and 3′ UTRs from bovine growth hormone, cytomegalovirus, mouse Hba-a1, HSD, an albumin gene, HBA, HBB, XBG, heat shock protein 90 (Hsp90), glyceraldehyde 3-phosphate dehydrogenase (GAPDH), beta-actin, alpha-tubulin, tumor protein (p53), or epidermal growth factor receptor (EGFR).

In some embodiments, the mRNA comprises 5′ and 3′ UTRs that are from the same source, e.g., a constitutively expressed mRNA such as actin, albumin, or a globin such as HBA, HBB, or XBG.

In some embodiments, the mRNA does not comprise a 5′ UTR, e.g., there are no additional nucleotides between the 5′ cap and the start codon. In some embodiments, the mRNA comprises a Kozak sequence (described below) between the 5′ cap and the start codon, but does not have any additional 5′ UTR. In some embodiments, the mRNA does not comprise a 3′ UTR, e.g., there are no additional nucleotides between the stop codon and the poly-A tail.

In some embodiments, the mRNA comprises a Kozak sequence. The Kozak sequence can affect translation initiation and the overall yield of a polypeptide translated from an mRNA. A Kozak sequence includes a methionine codon that can function as the start codon. A minimal Kozak sequence is NNNRUGN wherein at least one of the following is true: the first N is A or G and the second N is G. In the context of a nucleotide sequence, R means a purine (A or G). In some embodiments, the Kozak sequence is RNNRUGN, NNNRUGG, RNNRUGG, RNNAUGN, NNNAUGG, or RNNAUGG. In some embodiments, the Kozak sequence is rccRUGg with zero mismatches or with up to one or two mismatches to positions in lowercase. In some embodiments, the Kozak sequence is rccAUGg with zero mismatches or with up to one or two mismatches to positions in lowercase. In some embodiments, the Kozak sequence is gccRccAUGG (SEQ ID NO: 26) with zero mismatches or with up to one, two, or three mismatches to positions in lowercase. In some embodiments, the Kozak sequence is gccAccAUG with zero mismatches or with up to one, two, three, or four mismatches to positions in lowercase. In some embodiments, the Kozak sequence is GCCACCAUG. In some embodiments, the Kozak sequence is gccgccRccAUGG (SEQ ID NO: 27) with zero mismatches or with up to one, two, three, or four mismatches to positions in lowercase.

In some embodiments, the mRNA comprising an ORF encoding an RNA-guided DNA binding agent comprises a sequence having at least 90% identity to any of the Cas ORFs in Table 89.

In some embodiments, an mRNA disclosed herein comprises a 5′ cap, such as a Cap0, Cap1, or Cap2. A 5′ cap is generally a 7-methylguanine ribonucleotide (which may be further modified, as discussed below e.g. with respect to ARCA) linked through a 5′-triphosphate to the 5′ position of the first nucleotide of the 5′-to-3′ chain of the mRNA, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3′-O-methyl)GpppG and 7-methyl(3′deoxy)GpppG,” RNA 7: 1486-1495. The ARCA structure is shown below.

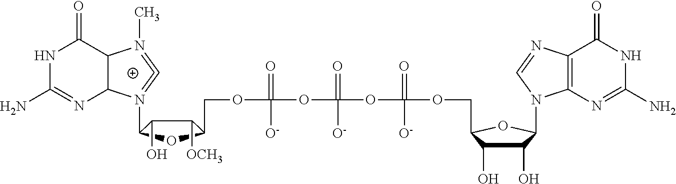

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below.

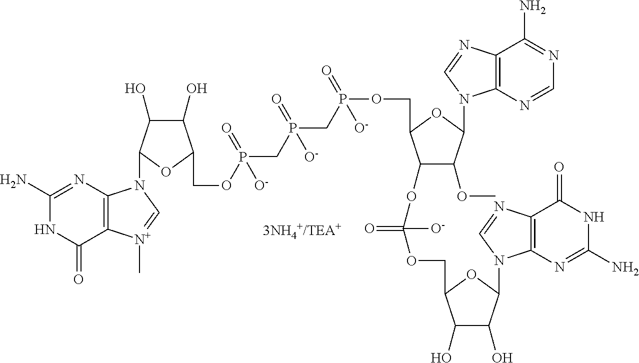

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479.

In some embodiments, the mRNA further comprises a poly-adenylated (poly-A) tail. In some embodiments, the poly-A tail comprises at least 20, 30, 40, 50, 60, 70, 80, 90, or 100 adenines, optionally up to 300 adenines. In some embodiments, the poly-A tail comprises 95, 96, 97, 98, 99, or 100 adenine nucleotides. In some instances, the poly-A tail is “interrupted” with one or more non-adenine nucleotide “anchors” at one or more locations within the poly-A tail. The poly-A tails may comprise at least 8 consecutive adenine nucleotides, but also comprise one or more non-adenine nucleotide. As used herein, “non-adenine nucleotides” refer to any natural or non-natural nucleotides that do not comprise adenine. Guanine, thymine, and cytosine nucleotides are exemplary non-adenine nucleotides. Thus, the poly-A tails on the mRNA described herein may comprise consecutive adenine nucleotides located 3′ to nucleotides encoding an RNA-guided DNA-binding agent or a sequence of interest. In some instances, the poly-A tails on mRNA comprise non-consecutive adenine nucleotides located 3′ to nucleotides encoding an RNA-guided DNA-binding agent or a sequence of interest, wherein non-adenine nucleotides interrupt the adenine nucleotides at regular or irregularly spaced intervals.

As used herein, “non-adenine nucleotides” refer to any natural or non-natural nucleotides that do not comprise adenine. Guanine, thymine, and cytosine nucleotides are exemplary non-adenine nucleotides. Thus, the poly-A tails on the mRNA described herein may comprise consecutive adenine nucleotides located 3′ to nucleotides encoding an RNA-guided DNA-binding agent or a sequence of interest. In some instances, the poly-A tails on mRNA comprise non-consecutive adenine nucleotides located 3′ to nucleotides encoding an RNA-guided DNA-binding agent or a sequence of interest, wherein non-adenine nucleotides interrupt the adenine nucleotides at regular or irregularly spaced intervals.

In some embodiments, the mRNA is purified. In some embodiments, the mRNA is purified using a precipitation method (e.g., LiCl precipitation, alcohol precipitation, or an equivalent method, e.g., as described herein). In some embodiments, the mRNA is purified using a chromatography-based method, such as an HPLC-based method or an equivalent method (e.g., as described herein). In some embodiments, the mRNA is purified using both a precipitation method (e.g., LiCl precipitation) and an HPLC-based method.

In some embodiments, at least one gRNA is provided in combination with an mRNA disclosed herein. In some embodiments, a gRNA is provided as a separate molecule from the mRNA. In some embodiments, a gRNA is provided as a part, such as a part of a UTR, of an mRNA disclosed herein.

B. Chemically Modified Nucleic Acids

In some embodiments, the nucleic acid is an RNA, such as a chemically modified RNA. In some embodiments, the nucleic acid is a DNA, or comprises DNA, such as a chemically modified DNA.

An RNA comprising one or more modified nucleosides or nucleotides is called a “modified” RNA or “chemically modified” RNA, to describe the presence of one or more non-naturally and/or naturally occurring components or configurations that are used instead of or in addition to the canonical A, G, C, and U residues. In some embodiments, a modified RNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

A gRNA comprising one or more modified nucleosides or nucleotides is called a “modified” gRNA or “chemically modified” RNA, to describe the presence of one or more non-naturally and/or naturally occurring components or configurations that are used instead of or in addition to the canonical A, G, C, and U residues. In some embodiments, a modified gRNA is synthesized with a non-canonical nucleoside or nucleotide, is here called “modified.”

Chemical modifications such as those listed above can be combined to provide modified nucleic acids, DNAs, RNAs, or gRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In some embodiments, all, or substantially all, of the phosphate groups of a gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the nucleic acid such as a gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the modified nucleic acids such as the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

In some embodiments, the disclosure comprises a sgRNA comprising one or more modifications within one or more of the following regions: the nucleotides at the 5′ terminus; the lower stem region; the bulge region; the upper stem region; the nexus region; the hairpin 1 region; the hairpin 2 region; and the nucleotides at the 3′ terminus. In some embodiments, the modification comprises a 2′-O-methyl (2′-O-Me) modified nucleotide. In some embodiments, the modification comprises a 2′-fluoro (2′-F) modified nucleotide. In some embodiments, the modification comprises a phosphorothioate (PS) bond between nucleotides.

In some embodiments, the first three or four nucleotides at the 5′ terminus, and the last three or four nucleotides at the 3′ terminus are modified. In some embodiments, the first four nucleotides at the 5′ terminus, and the last four nucleotides at the 3′ terminus are linked with phosphorothioate (PS) bonds. In some embodiments, the modification comprises 2′-O-Me. In some embodiments, the modification comprises 2′-F.

In some embodiments, the first four nucleotides at the 5′ terminus and the last four nucleotides at the 3′ terminus are linked with a PS bond, and the first three nucleotides at the 5′ terminus and the last three nucleotides at the 3′ terminus comprise 2′-O-Me modifications.

In some embodiments, the first four nucleotides at the 5′ terminus and the last four nucleotides at the 3′ terminus are linked with a PS bond, and the first three nucleotides at the 5′ terminus and the last three nucleotides at the 3′ terminus comprise 2′-F modifications.

In some embodiments, the sgRNA comprises the modification pattern of: (mN*mN*mN*GUUUUAGAmGmCmUmAmGmAmAmAm UmAmGmCAAGUUAAAAUAAGGCUAGUCCGUUAUCAmAmCmUmUmGmAmAmA mAmAmGmUmGmGmCmAmCmCmGmAmGmUmCmGmGmUmGmCmU*mU*mU*mU (SEQ ID NO: 28), where N is any natural or non-natural nucleotide. A, C, G, and U are an adenine nucleotide, a cytidine nucleotide, a guanine nucleotide, and a uridine nucleotide, respectively. In certain embodiments, A, C, G, and U are each independently a naturally or non-naturally occurring nucleotide with the indicate base. In certain embodiments, A, C, G, and U are RNA nucleotides. In some embodiments, the sgRNA comprises the sequence disclosed in the sentence preceding this one. In some embodiments, the sgRNA comprises 2′O-methyl modification of the first three residues at its 5′ end, with phosphorothioate linkages between residues 1-2, 2-3, and 3-4 of the RNA.

C. Template Nucleic Acid

The compositions and methods disclosed herein may include a donor nucleic acid, i.e., a template nucleic acid. The template may be used to alter or insert a nucleic acid sequence at or near a target site for a Cas nuclease. In some embodiments, the methods comprise introducing a template to the cell. In some embodiments, a single template may be provided. In other embodiments, two or more templates may be provided such that editing may occur at two or more target sites. For example, different templates may be provided to edit a single gene in a cell, or two different genes in a cell.

In some embodiments, the template may be used in homologous recombination. In some embodiments, the homologous recombination may result in the integration of the template sequence or a portion of the template sequence into the target nucleic acid molecule. In other embodiments, the template may be used in homology-directed repair, which involves DNA strand invasion at the site of the cleavage in the nucleic acid. In some embodiments, the homology-directed repair may result in including the template sequence in the edited target nucleic acid molecule. In yet other embodiments, the template may be used in gene editing mediated by non-homologous end joining. In some embodiments, the template sequence has no similarity to the nucleic acid sequence near the cleavage site. In some embodiments, the template or a portion of the template sequence is incorporated. In some embodiments, the template includes flanking inverted terminal repeat (ITR) sequences.

In some embodiments, the template may comprise a first homology arm and a second homology arm (also called a first and second nucleotide sequence) that are complementary to sequences located upstream and downstream of the cleavage site, respectively. Where a template contains two homology arms, each arm can be the same length or different lengths, and the sequence between the homology arms can be substantially similar or identical to the target sequence between the homology arms, or it can be entirely unrelated. In some embodiments, the degree of complementarity or percent identity between the first nucleotide sequence on the template and the sequence upstream of the cleavage site, and between the second nucleotide sequence on the template and the sequence downstream of the cleavage site, may permit homologous recombination, such as, e.g., high-fidelity homologous recombination, between the template and the target nucleic acid molecule. In some embodiments, the degree of complementarity may be about 50%, 55%, 60%, 65%, 70%, 75%, 80%, 85%, 90%, 95%, 97%, 98%, 99%, or 100%. In some embodiments, the degree of complementarity may be about 95%, 97%, 98%, 99%, or 100%. In some embodiments, the degree of complementarity may be at least 98%, 99%, or 100%. In some embodiments, the degree of complementarity may be 100%. In some embodiments, the percent identity may be about 50%, 55%, 60%, 65%, 70%, 75%, 80%, 85%, 90%, 95%, 97%, 98%, 99%, or 100%. In some embodiments, the percent identity may be about 95%, 97%, 98%, 99%, or 100%. In some embodiments, the percent identity may be at least 98%, 99%, or 100%. In some embodiments, the percent identity may be 100%.

In some embodiments, the template sequence may correspond to, comprise, or consist of an endogenous sequence of a target cell. It may also or alternatively correspond to, comprise, or consist of an exogenous sequence of a target cell. As used herein, the term “endogenous sequence” refers to a sequence that is native to the cell. The term “exogenous sequence” refers to a sequence that is not native to a cell, or a sequence whose native location in the genome of the cell is in a different location. In some embodiments, the endogenous sequence may be a genomic sequence of the cell. In some embodiments, the endogenous sequence may be a chromosomal or extrachromosomal sequence. In some embodiments, the endogenous sequence may be a plasmid sequence of the cell. In some embodiments, the template sequence may be substantially identical to a portion of the endogenous sequence in a cell at or near the cleavage site, but comprise at least one nucleotide change. In some embodiments, editing the cleaved target nucleic acid molecule with the template may result in a mutation comprising an insertion, deletion, or substitution of one or more nucleotides of the target nucleic acid molecule. In some embodiments, the mutation may result in one or more amino acid changes in a protein expressed from a gene comprising the target sequence.

In some embodiments, the mutation may result in one or more nucleotide changes in an RNA expressed from the target insertion site. In some embodiments, the mutation may alter the expression level of a target gene. In some embodiments, the mutation may result in increased or decreased expression of the target gene. In some embodiments, the mutation may result in gene knock-down. In some embodiments, the mutation may result in gene knock-out. In some embodiments, the mutation may result in restored gene function. In some embodiments, editing of the cleaved target nucleic acid molecule with the template may result in a change in an exon sequence, an intron sequence, a regulatory sequence, a transcriptional control sequence, a translational control sequence, a splicing site, or a non-coding sequence of the target nucleic acid molecule, such as DNA.

In other embodiments, the template sequence may comprise an exogenous sequence. In some embodiments, the exogenous sequence may comprise a coding sequence. In some embodiments, the exogenous sequence may comprise a protein or RNA coding sequence (e.g., an ORF) operably linked to an exogenous promoter sequence such that, upon integration of the exogenous sequence into the target nucleic acid molecule, the cell is capable of expressing the protein or RNA encoded by the integrated sequence. In other embodiments, upon integration of the exogenous sequence into the target nucleic acid molecule, the expression of the integrated sequence may be regulated by an endogenous promoter sequence. In some embodiments, the exogenous sequence may provide a cDNA sequence encoding a protein or a portion of the protein. In yet other embodiments, the exogenous sequence may comprise or consist of an exon sequence, an intron sequence, a regulatory sequence, a transcriptional control sequence, a translational control sequence, a splicing site, or a non-coding sequence. In some embodiments, the integration of the exogenous sequence may result in restored gene function. In some embodiments, the integration of the exogenous sequence may result in a gene knock-in. In some embodiments, the integration of the exogenous sequence may result in a gene knock-out.

The template may be of any suitable length. In some embodiments, the template may comprise 10, 15, 20, 25, 50, 75, 100, 150, 200, 500, 1000, 1500, 2000, 2500, 3000, 3500, 4000, 4500, 5000, 5500, 6000, or more nucleotides in length. The template may be a single-stranded nucleic acid. The template can be double-stranded or partially double-stranded nucleic acid. In some embodiments, the single stranded template is 20, 30, 40, 50, 75, 100, 125, 150, 175, or 200 nucleotides in length. In some embodiments, the template may comprise a nucleotide sequence that is complementary to a portion of the target nucleic acid molecule comprising the target sequence (i.e., a “homology arm”). In some embodiments, the template may comprise a homology arm that is complementary to the sequence located upstream or downstream of the cleavage site on the target nucleic acid molecule.

In some embodiments, the template contains ssDNA or dsDNA containing flanking invert-terminal repeat (ITR) sequences. In some embodiments, the template is provided as a vector, plasmid, minicircle, nanocircle, or PCR product.

D. Purification of Nucleic Acids

In some embodiments, the nucleic acid is purified. In some embodiments, the nucleic acid is purified using a precipitation method (e.g., LiCl precipitation, alcohol precipitation, or an equivalent method, e.g., as described herein). In some embodiments, the nucleic acid is purified using a chromatography-based method, such as an HPLC-based method or an equivalent method (e.g., as described herein). In some embodiments, the nucleic is purified using both a precipitation method (e.g., LiCl precipitation) and an HPLC-based method.

E. Target Sequences

In some embodiments, a CRISPR/Cas system of the present disclosure may be directed to and cleave a target sequence on a target nucleic acid molecule. For example, the target sequence may be recognized and cleaved by the Cas nuclease. In some embodiments, a target sequence for a Cas nuclease is located near the nuclease's cognate PAM sequence. In some embodiments, a Class 2 Cas nuclease may be directed by a gRNA to a target sequence of a target nucleic acid molecule, where the gRNA hybridizes with and the Class 2 Cas protein cleaves the target sequence. In some embodiments, the guide RNA hybridizes with and a Class 2 Cas nuclease cleaves the target sequence adjacent to or comprising its cognate PAM. In some embodiments, the target sequence may be complementary to the targeting sequence of the guide RNA. In some embodiments, the degree of complementarity between a targeting sequence of a guide RNA and the portion of the corresponding target sequence that hybridizes to the guide RNA may be about 50%, 55%, 60%, 65%, 70%, 75%, 80%, 85%, 90%, 95%, 97%, 98%, 99%, or 100%. In some embodiments, the percent identity between a targeting sequence of a guide RNA and the portion of the corresponding target sequence that hybridizes to the guide RNA may be about 50%, 55%, 60%, 65%, 70%, 75%, 80%, 85%, 90%, 95%, 97%, 98%, 99%, or 100%. In some embodiments, the homology region of the target is adjacent to a cognate PAM sequence. In some embodiments, the target sequence may comprise a sequence 100% complementary with the targeting sequence of the guide RNA. In other embodiments, the target sequence may comprise at least one mismatch, deletion, or insertion, as compared to the targeting sequence of the guide RNA.

The length of the target sequence may depend on the nuclease system used. For example, the targeting sequence of a guide RNA for a CRISPR/Cas system may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length and the target sequence is a corresponding length, optionally adjacent to a PAM sequence. In some embodiments, the target sequence may comprise 15-24 nucleotides in length. In some embodiments, the target sequence may comprise 17-21 nucleotides in length. In some embodiments, the target sequence may comprise 20 nucleotides in length. When nickases are used, the target sequence may comprise a pair of target sequences recognized by a pair of nickases that cleave opposite strands of the DNA molecule. In some embodiments, the target sequence may comprise a pair of target sequences recognized by a pair of nickases that cleave the same strands of the DNA molecule. In some embodiments, the target sequence may comprise a part of target sequences recognized by one or more Cas nucleases.

The target nucleic acid molecule may be any DNA or RNA molecule that is endogenous or exogenous to a cell. In some embodiments, the target nucleic acid molecule may be an episomal DNA, a plasmid, a genomic DNA, viral genome, mitochondrial DNA, or chromosomal DNA from a cell or in the cell. In some embodiments, the target sequence of the target nucleic acid molecule may be a genomic sequence from a cell or in a cell, including a human cell.

In further embodiments, the target sequence may be a viral sequence. In further embodiments, the target sequence may be a pathogen sequence. In yet other embodiments, the target sequence may be a synthesized sequence. In further embodiments, the target sequence may be a chromosomal sequence. In certain embodiments, the target sequence may comprise a translocation junction, e.g., a translocation associated with a cancer. In some embodiments, the target sequence may be on a eukaryotic chromosome, such as a human chromosome.

In some embodiments, the target sequence may be located in a coding sequence of a gene, an intron sequence of a gene, a regulatory sequence, a transcriptional control sequence of a gene, a translational control sequence of a gene, a splicing site or a non-coding sequence between genes. In some embodiments, the gene may be a protein coding gene. In other embodiments, the gene may be a non-coding RNA gene. In some embodiments, the target sequence may comprise all or a portion of a disease-associated gene. In some embodiments, the target sequence may be located in a non-genic functional site in the genome, for example a site that controls aspects of chromatin organization, such as a scaffold site or locus control region.

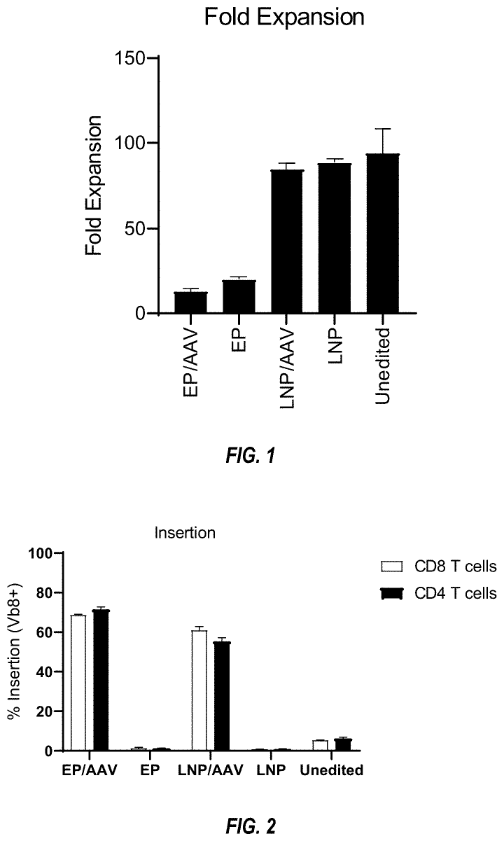

In some embodiments involving a Cas nuclease, such as a Class 2 Cas nuclease, the target sequence may be adjacent to a protospacer adjacent motif (“PAM”). In some embodiments, the PAM may be adjacent to or within 1, 2, 3, or 4, nucleotides of the 3′ end of the target sequence. The length and the sequence of the PAM may depend on the Cas protein used. For example, the PAM may be selected from a consensus or a particular PAM sequence for a specific Cas9 protein or Cas9 ortholog, including those disclosed in FIG. 1 of Ran et al., Nature, 520: 186-191 (2015), and Figure S5 of Zetsche 2015, the relevant disclosure of each of which is incorporated herein by reference. In some embodiments, the PAM may be 2, 3, 4, 5, 6, 7, 8, 9, or 10 nucleotides in length. Non-limiting exemplary PAM sequences include NGG, NGGNG, NG, NAAAAN, NNAAAAW, NNNNACA, GNNNCNNA, TTN, and NNNNGATT (wherein N is defined as any nucleotide, and W is defined as either A or T). In some embodiments, the PAM sequence may be NGG. In some embodiments, the PAM sequence may be NGGNG. In some embodiments, the PAM sequence may be TTN. In some embodiments, the PAM sequence may be NNAAAAW.

### VI. EXEMPLARY LIPID NUCLEIC ACID ASSEMBLIES

Disclosed herein are various embodiments using lipid nucleic acid assemblies comprising genome editing tools, such as RNAs, including CRISPR/Cas components and RNAs that express the same.

As used herein, “lipid nucleic acid assembly composition” refers to lipid-based delivery compositions, including lipid nanoparticles (LNPs) and lipoplexes. In some embodiments, “LNP compositions” are used interchangeably with “LNPs” or “LNP.”

In some embodiments, LNP refers to lipid nanoparticles with a diameter of <100 nM, or a population of LNP with an average diameter of <100 nM. In certain embodiments, an LNP has a diameter of about 1-250 nm, 10-200 nm, about 20-150 nm, about 50-150 nm, about 50-100 nm, about 50-120 nm, about 60-100 nm, about 75-150 nm, about 75-120 nm, or about 75-100 nm, or a population of the LNP with an average diameter of about 10-200 nm, about 20-150 nm, about 50-150 nm, about 50-100 nm, about 50-120 nm, about 60-100 nm, about 75-150 nm, about 75-120 nm, or about 75-100 nm. In preferred embodiments, an LNP composition has a diameter of 75-150 nm.

LNPs are formed by precise mixing a lipid component (e.g., in ethanol) with an aqueous nucleic acid component and LNPs are uniform in size. Lipoplexes are particles formed by bulk mixing the lipid and nucleic acid components and are between about 100 nm and 1 micron in size. In certain embodiments the lipid nucleic acid assemblies are LNPs. As used herein, a “lipid nucleic acid assembly” comprises a plurality of (i.e. more than one) lipid molecules physically associated with each other by intermolecular forces. A lipid nucleic acid assembly may comprise a bioavailable lipid having a pKa value of <7.5 or <7. The lipid nucleic acid assemblies are formed by mixing an aqueous nucleic acid-containing solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer may optionally be comprised in a pharmaceutical formulation comprising the lipid nucleic acid assemblies, e.g., for an ex vivo ACT therapy. In some embodiments, the aqueous solution comprises an RNA, such as an mRNA or a gRNA. In some embodiments, the aqueous solution comprises an mRNA encoding an RNA-guided DNA binding agent, such as Cas9.

In some embodiments, the lipid nucleic acid assembly formulations include an “amine lipid” (sometimes herein or elsewhere described as an “ionizable lipid” or a “biodegradable lipid”), together with an optional “helper lipid”, a “neutral lipid”, and a stealth lipid such as a PEG lipid. In some embodiments, the amine lipids or ionizable lipids are cationic depending on the pH.

A. Amine Lipids

In some embodiments, lipid nucleic acid assembly compositions comprise an “amine lipid”, which is, for example an ionizable lipid such as Lipid A, or Lipid D or their equivalents, including acetal analogs of Lipid A or Lipid D.

In some embodiments, the amine lipid is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

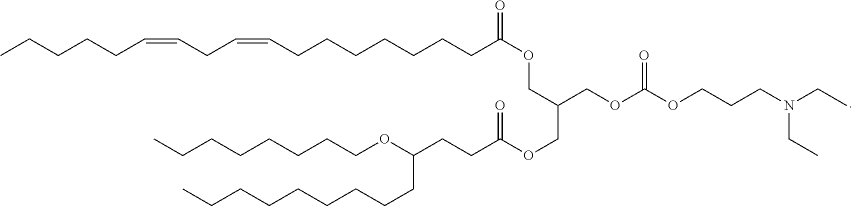 [SMILES: CCCCC/C=C\C/C=C\CCCCCCCC(=O)OCC(COC(=O)CCC(CCCCCCCCC)OCCCCCCCC)COC(=O)OCCCN(CC)CC]

Lipid A may be synthesized according to WO2015/095340 (e.g., pp. 84-86). In some embodiments, the amine lipid is Lipid A, or an amine lipid provided in WO2020/219876, which is hereby incorporated by reference.

In some embodiments, an amine lipid is an analog of Lipid A. In some embodiments, a Lipid A analog is an acetal analog of Lipid A. In particular lipid nucleic acid assembly compositions, the acetal analog is a C4-C12 acetal analog. In some embodiments, the acetal analog is a C5-C12 acetal analog. In additional embodiments, the acetal analog is a C5-C10 acetal analog. In further embodiments, the acetal analog is chosen from a C4, C5, C6, C7, C9, C10, C11, and C12 acetal analog.

In some embodiments, the amine lipid is a compound having a structure of Formula IA

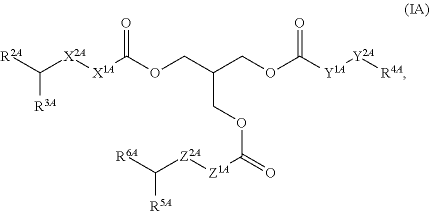

wherein
X1A is O, NH, or a direct bond;
X2A is C2-3 alkylene;
R3A is C1-3 alkyl;
R2A is C1-3 alkyl, or
R2A taken together with the nitrogen atom to which it is attached and 2-3 carbon atoms of X2A form a 5- or 6-membered ring, or
R2A taken together with R3A and the nitrogen atom to which they are attached form a 5-membered ring;
Y1A is C6-10 alkylene;
Y2A is selected from

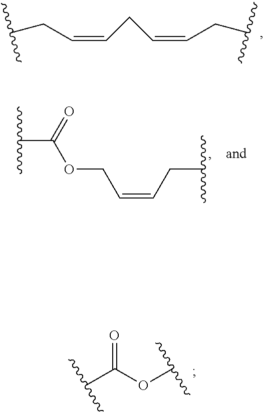 [SMILES: *C/C=C\C/C=C\C* ; *C/C=C\COC(*)=O ; *OC(*)=O]

R4A is C4-11 alkyl;
Z1A is C2-5 alkylene;

### Z2A is

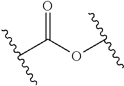 [SMILES: *OC(*)=O]

or absent;
R5A is C6-8 alkyl or C6-8 alkoxy; and
R6A is C6-8 alkyl or C6-8 alkoxy
or a salt thereof.

In some embodiments, the amine lipid is a compound of Formula (IIA)

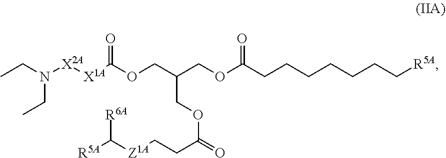

wherein
X1A is O, NH, or a direct bond;
X2A is C2-3 alkylene;
Z1A is C3 alkylene and R5A and R6A are each C6 alkyl, or Z1A is a direct bond and R5A and
R6A are each C8 alkoxy; and

### R8A is

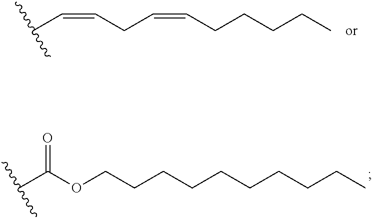 [SMILES: */C=C\C/C=C\CCCCC ; *C(=O)OCCCCCCCCCC]

or a salt thereof.

In certain embodiments, X1A is 0. In other embodiments, X1A is NH. In still other embodiments, X1A is a direct bond.

In certain embodiments, X2A is C3 alkylene. In particular embodiments, X2A is C2 alkylene.

In certain embodiments, Z1A is a direct bond and R5A and R6A are each C8 alkoxy. In other embodiments, Z1A is C3 alkylene and R5A and R6A are each C6 alkyl.

In certain embodiments, R8A is

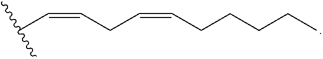 [SMILES: */C=C\C/C=C\CCCCC]

In other embodiments, R8A is

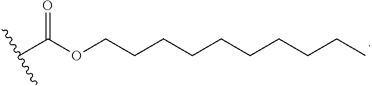 [SMILES: *C(=O)OCCCCCCCCCC]

In certain embodiments, the amine lipid is a salt.

Representative compounds of Formula (IA) include:

| Com- | |
|---|---|
| pound | |
| Num- | |
| ber | Compound |
| 1A | 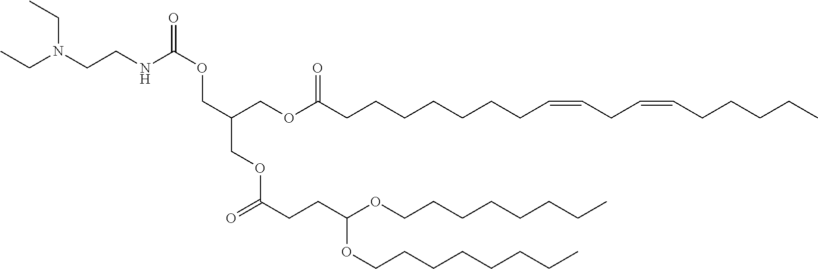 [SMILES: CCCCC/C=C\C/C=C\CCCCCCCC(=O)OCC(COC(=O)CCC(OCCCCCCCC)OCCCCCCCC)COC(=O)NCCN(CC)CC] |
| 2A | 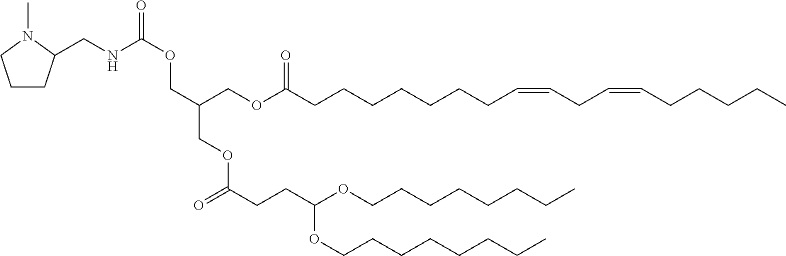 [SMILES: CCCCC/C=C\C/C=C\CCCCCCCC(=O)OCC(COC(=O)CCC(OCCCCCCCC)OCCCCCCCC)COC(=O)NCC1CCCN1C] |
| 3A | 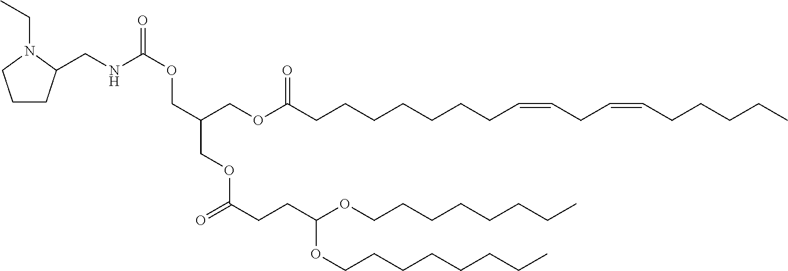 [SMILES: CCCCC/C=C\C/C=C\CCCCCCCC(=O)OCC(COC(=O)CCC(OCCCCCCCC)OCCCCCCCC)COC(=O)NCC1CCCN1CC] |
| 4A | 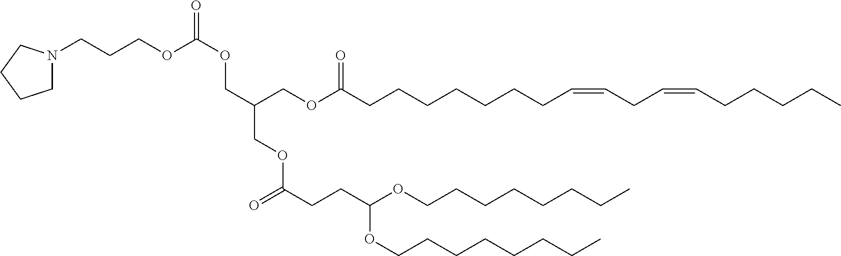 [SMILES: CCCCC/C=C\C/C=C\CCCCCCCC(=O)OCC(COC(=O)CCC(OCCCCCCCC)OCCCCCCCC)COC(=O)OCCCN1CCCC1] |
| 5A | 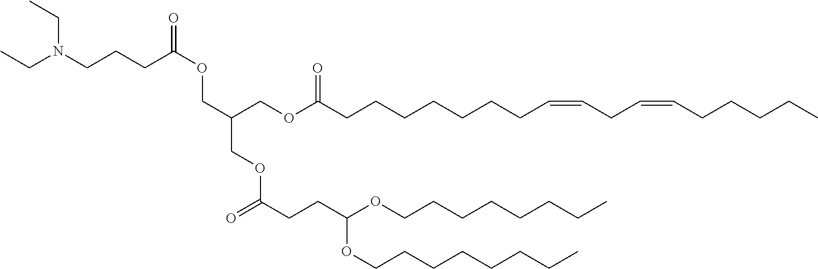 [SMILES: CCCCC/C=C\C/C=C\CCCCCCCC(=O)OCC(COC(=O)CCCN(CC)CC)COC(=O)CCC(OCCCCCCCC)OCCCCCCCC] |
| 6A | 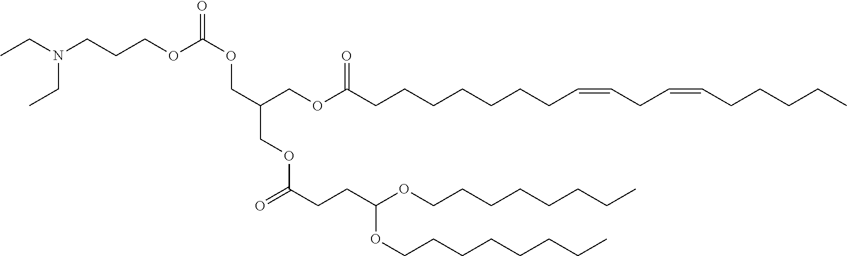 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| 7A | 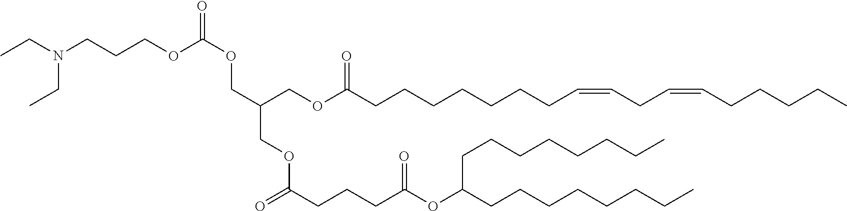 [SMILES: CCCCC/C=C\C/C=C\CCCCCCCC(=O)OCC(COC(=O)CCCC(=O)OC(CCCCCCCC)CCCCCCCC)COC(=O)OCCCN(CC)CC] |
| 8A | 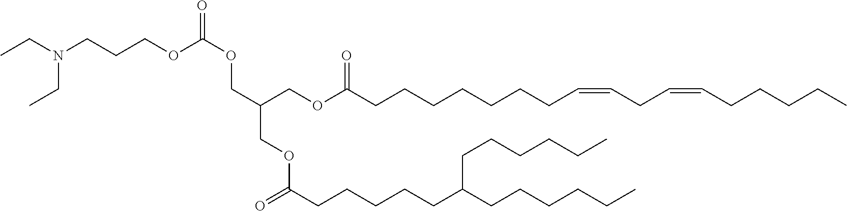 [SMILES: CCCCC/C=C\C/C=C\CCCCCCCC(=O)OCC(COC(=O)CCCCCC(CCCCCC)CCCCCC)COC(=O)OCCCN(CC)CC] |
| 9A | 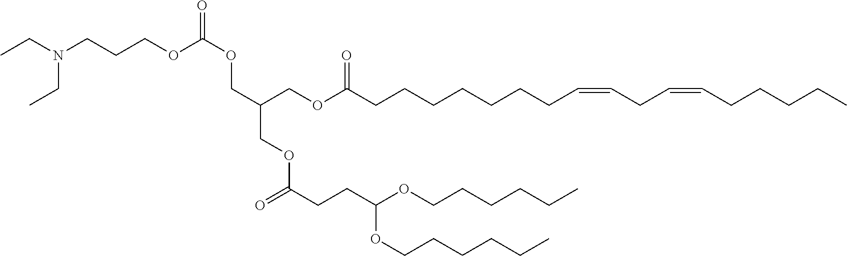 [SMILES: CCCCC/C=C\C/C=C\CCCCCCCC(=O)OCC(COC(=O)CCC(OCCCCCC)OCCCCCC)COC(=O)OCCCN(CC)CC] |
| 10A | 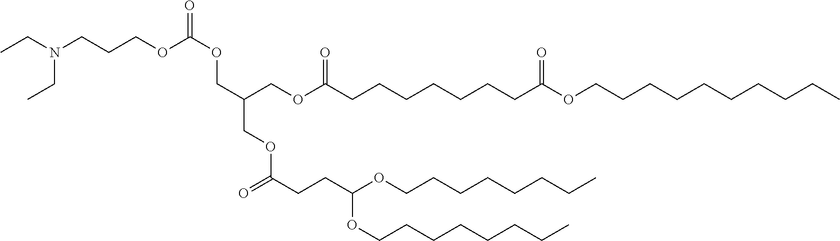 [SMILES: CCCCCCCCCCOC(=O)CCCCCCCC(=O)OCC(COC(=O)CCC(OCCCCCCCC)OCCCCCCCC)COC(=O)OCCCN(CC)CC] |
| 11A | 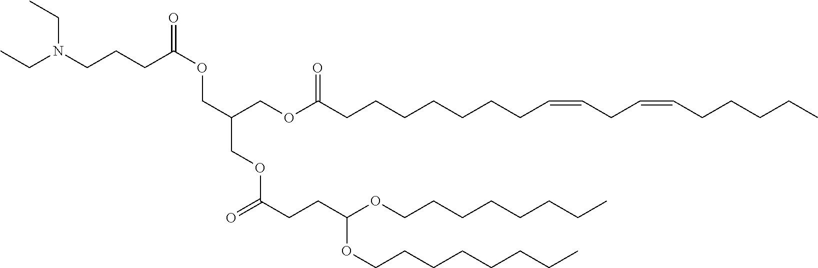 [SMILES: CCCCC/C=C\C/C=C\CCCCCCCC(=O)OCC(COC(=O)CCCN(CC)CC)COC(=O)CCC(OCCCCCCCC)OCCCCCCCC] |
| 12A | 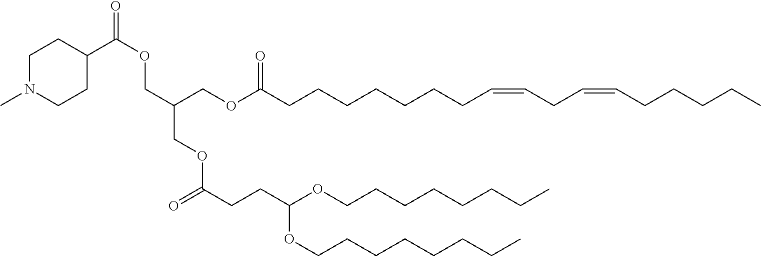 [SMILES: CCCCC/C=C\C/C=C\CCCCCCCC(=O)OCC(COC(=O)CCC(OCCCCCCCC)OCCCCCCCC)COC(=O)C1CCN(C)CC1] |
| 13A | 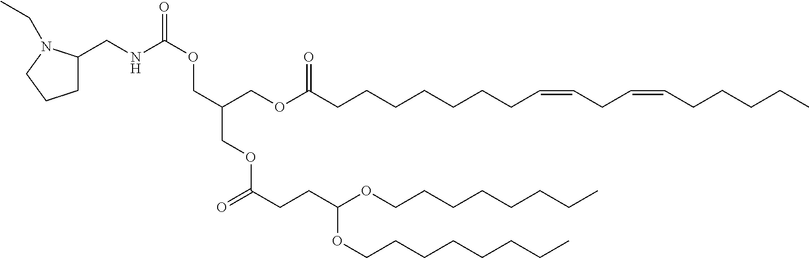 [SMILES: CCCCC/C=C\C/C=C\CCCCCCCC(=O)OCC(COC(=O)CCC(OCCCCCCCC)OCCCCCCCC)COC(=O)NCC1CCCN1CC] |
| 14A | 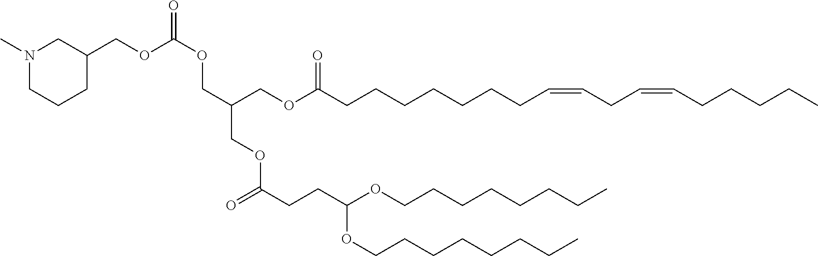 [SMILES: CCCCC/C=C\C/C=C\CCCCCCCC(=O)OCC(COC(=O)CCC(OCCCCCCCC)OCCCCCCCC)COC(=O)OCC1CCCN(C)C1] |
| 15A | 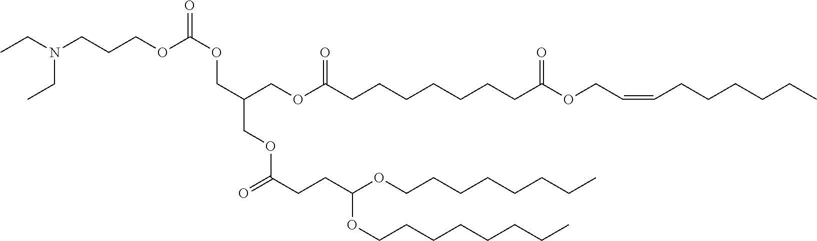 [SMILES: CCCCCC/C=C\COC(=O)CCCCCCCC(=O)OCC(COC(=O)CCC(OCCCCCCCC)OCCCCCCCC)COC(=O)OCCCN(CC)CC] |
| 16A | 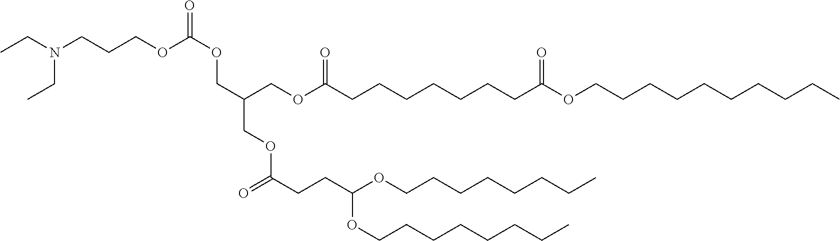 [SMILES: CCCCCCCCCCOC(=O)CCCCCCCC(=O)OCC(COC(=O)CCC(OCCCCCCCC)OCCCCCCCC)COC(=O)OCCCN(CC)CC] |
| 17A | 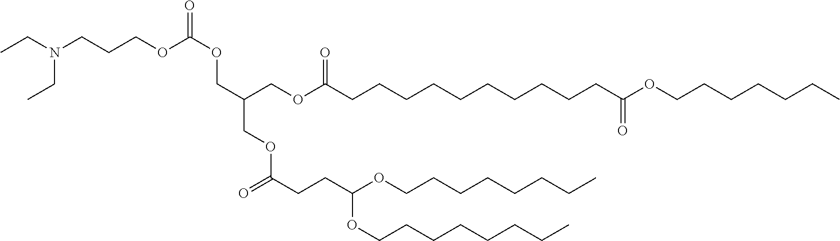 [SMILES: CCCCCCCCOC(CCC(=O)OCC(COC(=O)CCCCCCCCCCC(=O)OCCCCCCC)COC(=O)OCCCN(CC)CC)OCCCCCCCC] |
| 18A | 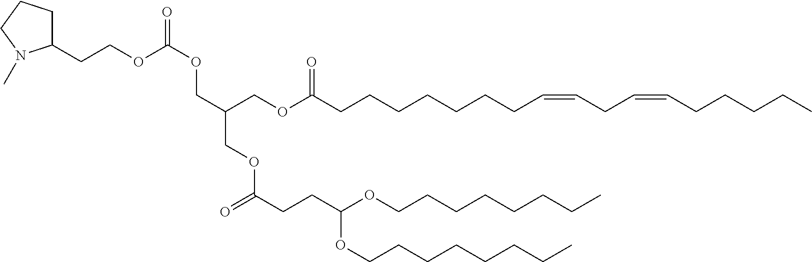 [SMILES: CCCCC/C=C\C/C=C\CCCCCCCC(=O)OCC(COC(=O)CCC(OCCCCCCCC)OCCCCCCCC)COC(=O)OCCC1CCCN1C] |
| 19A | 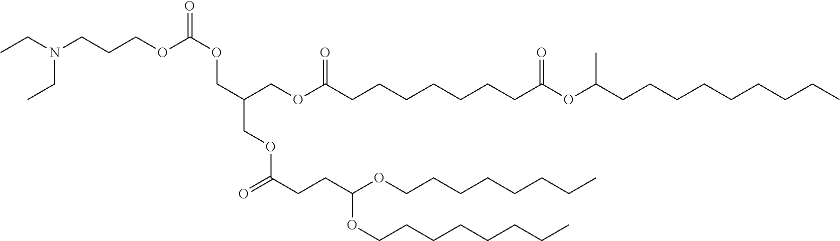 [SMILES: CCCCCCCCCC(C)OC(=O)CCCCCCCC(=O)OCC(COC(=O)CCC(OCCCCCCCC)OCCCCCCCC)COC(=O)OCCCN(CC)CC] |

or a salt thereof, such as a pharmaceutically acceptable salt thereof.

In some embodiments, the amine lipid is Lipid D, which is nonyl 84(7,7-bis(octyloxy)heptyl)(2-hydroxyethyl)amino)octanoate:

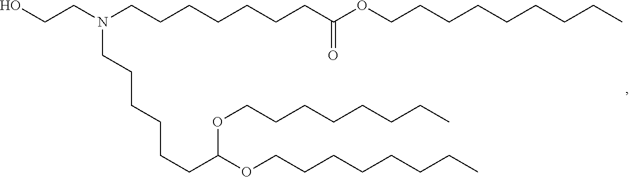 [SMILES: CCCCCCCCCOC(=O)CCCCCCCN(CCO)CCCCCCC(OCCCCCCCC)OCCCCCCCC]

or a salt thereof.

Lipid D may be synthesized according to WO2020072605 and Mol. Ther. 2018, 26(6), 1509-1519 (“Sabnis”), which are incorporated by reference in their entireties. In some embodiments, the amine lipid Lipid D, or an amine lipid provided in WO2020072605, which is hereby incorporated by reference.

In some embodiments, the amine lipid is a compound having a structure of Formula IB:

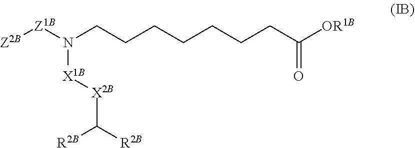

wherein
X^{1B} is C₆₋₇ alkylene;

### X2B is

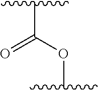 [SMILES: *OC(*)=O]

or absent, provided that if X^{2B} is

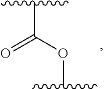 [SMILES: *OC(*)=O]

R^{2B} is not alkoxy;
Z^{1B} is C₂₋₃ alkylene;
Z^{2B} is selected from —OH, —NHC(═O)OCH₃, and —NHS(═O)₂CH₃;
R^{1B} is C₇₋₉ unbranched alkyl; and
each R^{2B} is independently C₈ alkyl or C₈ alkoxy;
or a salt thereof.

In some embodiments, the amine lipid is a compound of Formula (JIB)

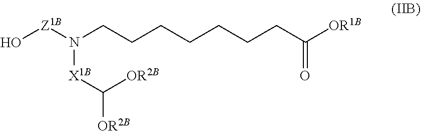

wherein
X^{1B} is C₆₋₇ alkylene;
Z^{1B} is C₂₋₃ alkylene;
R^{1B} is C₇₋₉ unbranched alkyl; and
each R^{2B} is C₈ alkyl;
or a salt thereof.

In certain embodiments, X^{1B} is C₆ alkylene. In other embodiments, X^{1B} is C₇ alkylene.

In certain embodiments, Z^{1B} is a direct bond and R^{5B} and R^{6B} are each C₈ alkoxy. In other embodiments, Z^{1B} is C₃ alkylene and R^{5B} and R^{6B} are each C₆ alkyl.

In certain embodiments, X^{2B} is

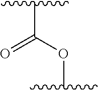 [SMILES: *OC(*)=O]

and R^{2B} is not alkoxy. In other embodiments, X^{2B} is absent.

In certain embodiments, Z^{1B} is C₂ alkylene; In other embodiments, Z^{1B} is C₃ alkylene.

In certain embodiments, Z^{2B} is —OH. In other embodiments, Z^{2B} is —NHC(═O)OCH₃. In other embodiments, Z^{2B} is —NHS(═O)₂CH₃.

In certain embodiments, R^{1B} is C₇ unbranched alkylene. In other embodiments, R^{1B} is C₈ branched or unbranched alkylene. In other embodiments, R^{1B} is C₉ branched or unbranched alkylene.

In certain embodiments, the amine lipid is a salt.

Representative compounds of Formula (IB) include:

| Compound | |
|---|---|
| Number | Compound |
| 1B | 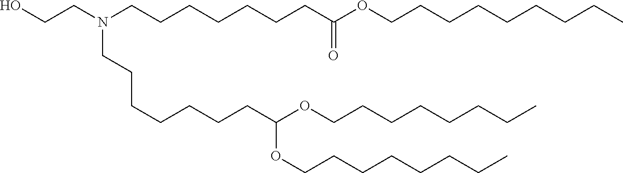 [SMILES: CCCCCCCCCOC(=O)CCCCCCCN(CCO)CCCCCCCC(OCCCCCCCC)OCCCCCCCC] |
| 2B | 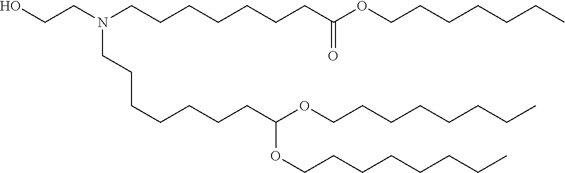 [SMILES: CCCCCCCCOC(CCCCCCCN(CCO)CCCCCCCC(=O)OCCCCCCC)OCCCCCCCC] |
| 3B | 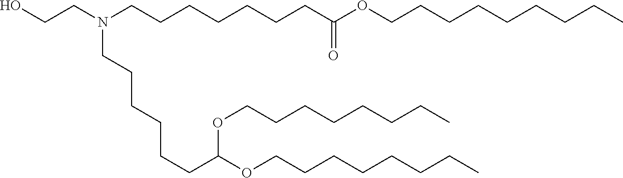 [SMILES: CCCCCCCCCOC(=O)CCCCCCCN(CCO)CCCCCCC(OCCCCCCCC)OCCCCCCCC] |
| 4B | 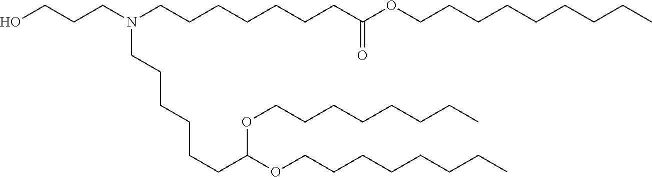 [SMILES: CCCCCCCCCOC(=O)CCCCCCCN(CCCO)CCCCCCC(OCCCCCCCC)OCCCCCCCC] |
| 5B | 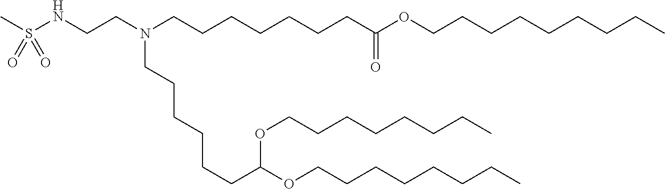 [SMILES: CCCCCCCCCOC(=O)CCCCCCCN(CCCCCCC(OCCCCCCCC)OCCCCCCCC)CCNS(C)(=O)=O] |
| 6B | 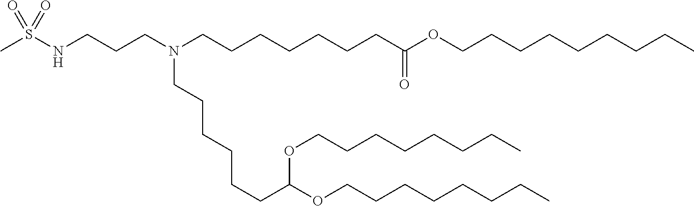 [SMILES: CCCCCCCCCOC(=O)CCCCCCCN(CCCCCCC(OCCCCCCCC)OCCCCCCCC)CCCNS(C)(=O)=O] |
| 7B | 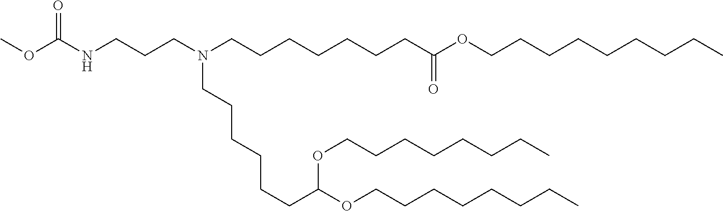 [SMILES: CCCCCCCCCOC(=O)CCCCCCCN(CCCCCCC(OCCCCCCCC)OCCCCCCCC)CCCNC(=O)OC] |

or a salt thereof, such as a pharmaceutically acceptable salt thereof.

Amine lipids and other “biodegradable lipids” suitable for use in the lipid nucleic acid assemblies described herein are biodegradable in vivo or ex vivo. The amine lipids have low toxicity (e.g., are tolerated in animal models without adverse effect in amounts of greater than or equal to 10 mg/kg). In some embodiments, lipid nucleic acid assemblies comprising an amine lipid include those where at least 75% of the amine lipid is cleared from the plasma or the engineered cell within 8, 10, 12, 24, or 48 hours, or 3, 4, 5, 6, 7, or 10 days. In some embodiments, lipid nucleic acid assemblies comprising an amine lipid include those where at least 50% of the nucleic acid, e.g., mRNA or gRNA, is cleared from the plasma within 8, 10, 12, 24, or 48 hours, or 3, 4, 5, 6, 7, or 10 days. In some embodiments, lipid nucleic acid assemblies comprising an amine lipid include those where at least 50% of the lipid nucleic acid assembly is cleared from the plasma within 8, 10, 12, 24, or 48 hours, or 3, 4, 5, 6, 7, or 10 days, for example by measuring a lipid (e.g. an amine lipid), nucleic acid, e.g., RNA/mRNA, or other component. In some embodiments, lipid-encapsulated versus free lipid, RNA, or nucleic acid component of the lipid nucleic acid assembly is measured.

Biodegradable lipids include, for example the biodegradable lipids of WO/2020/219876 (e.g., at pp. 13-33, 66-87), WO/2020/118041, WO/2020/072605 (e.g., at pp. 5-12, 21-29, 61-68, WO/2019/067992, WO/2017/173054, WO2015/095340, and WO2014/136086, and LNPs include LNP compositions described therein, the lipids and compositions of which are hereby incorporated by reference.

Lipid clearance may be measured as described in literature. See Maier, M. A., et al. Biodegradable Lipids Enabling Rapidly Eliminated Lipid Nanoparticles for Systemic Delivery of RNAi Therapeutics. Mol. Ther. 2013, 21(8), 1570-78 (“Maier”). For example, in Maier, LNP-siRNA systems containing luciferases-targeting siRNA were administered to six- to eight-week old male C57Bl/6 mice at 0.3 mg/kg by intravenous bolus injection via the lateral tail vein. Blood, liver, and spleen samples were collected at 0.083, 0.25, 0.5, 1, 2, 4, 8, 24, 48, 96, and 168 hours post-dose. Mice were perfused with saline before tissue collection and blood samples were processed to obtain plasma. All samples were processed and analyzed by LC-MS. Further, Maier describes a procedure for assessing toxicity after administration of LNP-siRNA formulations. For example, a luciferase-targeting siRNA was administered at 0, 1, 3, 5, and 10 mg/kg (5 animals/group) via single intravenous bolus injection at a dose volume of 5 mL/kg to male Sprague-Dawley rats. After 24 hours, about 1 mL of blood was obtained from the jugular vein of conscious animals and the serum was isolated. At 72 hours post-dose, all animals were euthanized for necropsy. Assessments of clinical signs, body weight, serum chemistry, organ weights and histopathology were performed. Although Maier describes methods for assessing siRNA-LNP formulations, these methods may be applied to assess clearance, pharmacokinetics, and toxicity of administration of lipid nucleic acid assembly compositions of the present disclosure.

Ionizable and bioavailable lipids for LNP delivery of nucleic acids known in the art are suitable. Lipids may be ionizable depending upon the pH of the medium they are in. For example, in a slightly acidic medium, the lipid, such as an amine lipid, may be protonated and thus bear a positive charge. Conversely, in a slightly basic medium, such as, for example, blood where pH is approximately 7.35, the lipid, such as an amine lipid, may not be protonated and thus bear no charge.

The ability of a lipid to bear a charge is related to its intrinsic pKa. In some embodiments, the amine lipids of the present disclosure may each, independently, have a pKa in the range of from about 5.1 to about 7.4. In some embodiments, the bioavailable lipids of the present disclosure may each, independently, have a pKa in the range of from about 5.1 to about 7.4, such as from about 5.5 to about 6.6, from about 5.6 to about 6.4, from about 5.8 to about 6.2, or from about 5.8 to about 6.5. For example, the amine lipids of the present disclosure may each, independently, have a pKa in the range of from about 5.8 to about 6.5. Lipids with a pKa ranging from about 5.1 to about 7.4 are effective for delivery of cargo in vivo, e.g. to the liver. Further, it has been found that lipids with a pKa ranging from about 5.3 to about 6.4 are effective for delivery in vivo, e.g. to tumors. See, e.g., WO2014/136086.

B. Additional Lipids

“Neutral lipids” suitable for use in a lipid composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE). In another embodiment, the neutral phospholipid may be distearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In one embodiment, the helper lipid may be cholesterol. In one embodiment, the helper lipid may be cholesterol hemisuccinate.

“Stealth lipids” are lipids that alter the length of time the nanoparticles can exist in vivo (e.g., in the blood). Stealth lipids may assist in the formulation process by, for example, reducing particle aggregation and controlling particle size. Stealth lipids used herein may modulate pharmacokinetic properties of the lipid nucleic acid assembly or aid in stability of the nanoparticle ex vivo. Stealth lipids suitable for use in a lipid composition of the disclosure include, but are not limited to, stealth lipids having a hydrophilic head group linked to a lipid moiety. Stealth lipids suitable for use in a lipid composition of the present disclosure and information about the biochemistry of such lipids can be found in Romberg et al., Pharmaceutical Research, Vol. 25, No. 1, 2008, pg. 55-71 and Hoekstra et al., Biochimica et Biophysica Acta 1660 (2004) 41-52. Additional suitable PEG lipids are disclosed, e.g., in WO 2006/007712.

In one embodiment, the hydrophilic head group of stealth lipid comprises a polymer moiety selected from polymers based on PEG. Stealth lipids may comprise a lipid moiety. In some embodiments, the stealth lipid is a PEG lipid.

In one embodiment, a stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly[N-(2-hydroxypropyl)methacrylamide].

In one embodiment, the PEG lipid comprises a polymer moiety based on PEG (sometimes referred to as poly(ethylene oxide)).

The PEG lipid further comprises a lipid moiety. In some embodiments, the lipid moiety may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. In some embodiments, the alkyl chain length comprises about C10 to C20. The dialkylglycerol or dialkylglycamide group can further comprise one or more substituted alkyl groups. The chain lengths may be symmetrical or asymmetrical.

Unless otherwise indicated, the term “PEG” as used herein means any polyethylene glycol or other polyalkylene ether polymer. In one embodiment, PEG is an optionally substituted linear or branched polymer of ethylene glycol or ethylene oxide. In one embodiment, PEG is unsubstituted. In one embodiment, the PEG is substituted, e.g., by one or more alkyl, alkoxy, acyl, hydroxy, or aryl groups. In one embodiment, the term includes PEG copolymers such as PEG-polyurethane or PEG-polypropylene (see, e.g., J. Milton Harris, Poly(ethylene glycol) chemistry: biotechnical and biomedical applications (1992)); in another embodiment, the term does not include PEG copolymers. In one embodiment, the PEG has a molecular weight of from about 130 to about 50,000, in a sub-embodiment, about 150 to about 30,000, in a sub-embodiment, about 150 to about 20,000, in a sub-embodiment about 150 to about 15,000, in a sub-embodiment, about 150 to about 10,000, in a sub-embodiment, about 150 to about 6,000, in a sub-embodiment, about 150 to about 5,000, in a sub-embodiment, about 150 to about 4,000, in a sub-embodiment, about 150 to about 3,000, in a sub-embodiment, about 300 to about 3,000, in a sub-embodiment, about 1,000 to about 3,000, and in a sub-embodiment, about 1,500 to about 2,500.

In some embodiments, the PEG (e.g., conjugated to a lipid moiety or lipid, such as a stealth lipid), is a “PEG-2K,” also termed “PEG 2000,” which has an average molecular weight of about 2,000 Daltons. PEG-2K is represented herein by the following formula (IV), wherein n is 45, meaning that the number averaged degree of polymerization comprises about 45 subunits

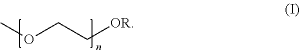

However, other PEG embodiments known in the art may be used, including, e.g., those where the number-averaged degree of polymerization comprises about 23 subunits (n=23), and/or 68 subunits (n=68). In some embodiments, n may range from about 30 to about 60. In some embodiments, n may range from about 35 to about 55. In some embodiments, n may range from about 40 to about 50. In some embodiments, n may range from about 42 to about 48. In some embodiments, n may be 45. In some embodiments, R may be selected from H, substituted alkyl, and unsubstituted alkyl. In some embodiments, R may be unsubstituted alkyl. In some embodiments, R may be methyl.

In any of the embodiments described herein, the PEG lipid may be selected from PEG-dilauroylglycerol, PEG-dimyristoylglycerol (PEG-DMG) (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-distearoylglycamide, PEG-cholesterol (1-[8′-(Cholest-5-en-3 [beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMG) (cat. #880150P from Avanti Polar Lipids, Alabaster, Ala., USA), 1,2-distearoyl-sn-glycero-3-phosphoethanolamine-N-[methoxy(polyethylene glycol)-2000] (PEG2k-DSPE) (cat. #880120C from Avanti Polar Lipids, Alabaster, Ala., USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-2000] (PEG2k-DSA). In one embodiment, the PEG lipid may be PEG2k-DMG. In some embodiments, the PEG lipid may be PEG2k-DSG. In one embodiment, the PEG lipid may be PEG2k-DSPE. In one embodiment, the PEG lipid may be PEG2k-DMA. In one embodiment, the PEG lipid may be PEG2k-C-DMA. In one embodiment, the PEG lipid may be compound 5027, disclosed in WO2016/010840 (paragraphs [00240] to [00244]). In one embodiment, the PEG lipid may be PEG2k-DSA. In one embodiment, the PEG lipid may be PEG2k-C11. In some embodiments, the PEG lipid may be PEG2k-C14. In some embodiments, the PEG lipid may be PEG2k-C16. In some embodiments, the PEG lipid may be PEG2k-C18.

C. Lipid Nucleic Acid Assembly Compositions

The lipid nucleic acid assembly may contain (i) a biodegradable lipid, (ii) an optional neutral lipid, (iii) a helper lipid, and (iv) a stealth lipid, such as a PEG lipid. The lipid nucleic acid assembly may contain a biodegradable lipid and one or more of a neutral lipid, a helper lipid, and a stealth lipid, such as a PEG lipid.

The lipid nucleic acid assembly may contain (i) an amine lipid for encapsulation and for endosomal escape, (ii) a neutral lipid for stabilization, (iii) a helper lipid, also for stabilization, and (iv) a stealth lipid, such as a PEG lipid. The lipid nucleic acid assembly may contain an amine lipid and one or more of a neutral lipid, a helper lipid, also for stabilization, and a stealth lipid, such as a PEG lipid.

A lipid nucleic acid assembly composition may comprise a nucleic acid, e.g., an RNA, component that includes one or more of an RNA-guided DNA-binding agent, a Cas nuclease mRNA, a Class 2 Cas nuclease mRNA, a Cas9 mRNA, and a gRNA. In some embodiments, a lipid nucleic acid assembly composition may include a Class 2 Cas nuclease and a gRNA as the RNA component. In some embodiments, n lipid nucleic acid assembly composition may comprise the RNA component, an amine lipid, a helper lipid, a neutral lipid, and a stealth lipid. In certain lipid nucleic acid assembly compositions, the helper lipid is cholesterol. In other compositions, the neutral lipid is DSPC. In additional embodiments, the stealth lipid is PEG2k-DMG or PEG2k-C11. In some embodiments, the lipid nucleic acid assembly composition comprises Lipid A or an equivalent of Lipid A; a helper lipid; a neutral lipid; a stealth lipid; and an RNA such as a gRNA. In some embodiments, the lipid nucleic acid assembly composition comprises Lipid A or an equivalent of Lipid A; a helper lipid; a stealth lipid; and an RNA such as a gRNA. In some compositions, the amine lipid is Lipid A. In some compositions, the amine lipid is Lipid A or an acetal analog thereof; the helper lipid is cholesterol; the neutral lipid is DSPC; and the stealth lipid is PEG2k-DMG.

In some embodiments, lipid compositions are described according to the respective molar ratios of the component lipids in the formulation. Embodiments of the present disclosure provide lipid compositions described according to the respective molar ratios of the component lipids in the formulation. In one embodiment, the mol % of the amine lipid may be from about 30 mol % to about 60 mol %. In one embodiment, the mol % of the amine lipid may be from about 40 mol % to about 60 mol %. In one embodiment, the mol % of the amine lipid may be from about 45 mol % to about 60 mol %. In one embodiment, the mol % of the amine lipid may be from about 50 mol % to about 60 mol %. In one embodiment, the mol % of the amine lipid may be from about 55 mol % to about 60 mol %. In one embodiment, the mol % of the amine lipid may be from about 50 mol % to about 55 mol %. In one embodiment, the mol % of the amine lipid may be about 50 mol %. In one embodiment, the mol % of the amine lipid may be about 55 mol %. In some embodiments, the amine lipid mol % of the lipid nucleic acid assembly batch will be ±30%, ±25%, ±20%, ±15%, ±10%, ±5%, or ±2.5% of the target mol %. In some embodiments, the amine lipid mol % of the lipid nucleic acid assembly batch will be ±4 mol %, ±3 mol %, ±2 mol %, ±1.5 mol %, ±1 mol %, ±0.5 mol %, or ±0.25 mol % of the target mol %. All mol % numbers are given as a fraction of the lipid component of the lipid nucleic acid assembly compositions. In some embodiments, lipid nucleic acid assembly inter-lot variability of the amine lipid mol % will be less than 15%, less than 10% or less than 5%.

In one embodiment, the mol % of the neutral lipid may be from about 5 mol % to about 15 mol %. In one embodiment, the mol % of the neutral lipid may be from about 7 mol % to about 12 mol %. In one embodiment, the mol % of the neutral lipid may be about 9 mol %. In some embodiments, the neutral lipid mol % of the lipid nucleic acid assembly batch will be ±30%, ±25%, ±20%, ±15%, ±10%, ±5%, or ±2.5% of the target neutral lipid mol %. In some embodiments, lipid nucleic acid assembly inter-lot variability will be less than 15%, less than 10% or less than 5%.

In one embodiment, the mol % of the helper lipid may be from about 20 mol % to about 60 mol %. In one embodiment, the mol % of the helper lipid may be from about 25 mol % to about 55 mol %. In one embodiment, the mol % of the helper lipid may be from about 25 mol % to about 50 mol %. In one embodiment, the mol % of the helper lipid may be from about 25 mol % to about 40 mol %. In one embodiment, the mol % of the helper lipid may be from about 30 mol % to about 50 mol %. In one embodiment, the mol % of the helper lipid may be from about 30 mol % to about 40 mol %. In one embodiment, the mol % of the helper lipid is adjusted based on amine lipid, neutral lipid, and PEG lipid concentrations to bring the lipid component to 100 mol %. In some embodiments, the helper mol % of the lipid nucleic acid assembly batch will be ±30%, ±25%, ±20%, ±15%, ±10%, ±5%, or ±2.5% of the target mol %. In some embodiments, lipid nucleic acid assembly inter-lot variability will be less than 15%, less than 10% or less than 5%.

In one embodiment, the mol % of the PEG lipid may be from about 1 mol % to about 10 mol %. In one embodiment, the mol % of the PEG lipid may be from about 2 mol % to about 10 mol %. In one embodiment, the mol % of the PEG lipid may be from about 1 mol % to about 3 mol %. In one embodiment, the mol % of the PEG lipid may be from about 2 mol % to about 4 mol %. In one embodiment, the mol % of the PEG lipid may be from about 1.5 mol % to about 2 mol %. In one embodiment, the mol % of the PEG lipid may be from about 2.5 mol % to about 4 mol %. In one embodiment, the mol % of the PEG lipid may be about 3 mol %. In one embodiment, the mol % of the PEG lipid may be about 2.5 mol %. In one embodiment, the mol % of the PEG lipid may be about 2 mol %. In one embodiment, the mol % of the PEG lipid may be about 1.5 mol %. In some embodiments, the PEG lipid mol % of the lipid nucleic acid assembly batch will be ±30%, ±25%, ±20%, ±15%, ±10%, ±5%, or ±2.5% of the target PEG lipid mol %. In some embodiments, lipid nucleic acid assembly composition, e.g. the LNP composition, inter-lot variability will be less than 15%, less than 10% or less than 5%.

Embodiments of the present disclosure provide LNP compositions, for example, LNP compositions comprising an ionizable lipid (e.g., Lipid A or one of its analogs), a helper lipid, a helper lipid, and a PEG lipid, described according to the respective molar ratios of the component lipids in the formulation. In certain embodiments, the amount of the ionizable lipid is from about 25 mol % to about 45 mol %; the amount of the neutral lipid is from about 10 mol % to about 30 mol %; the amount of the helper lipid is from about 25 mol % to about 65 mol %; and the amount of the PEG lipid is from about 1.5 mol % to about 3.5 mol %. In certain embodiments, the amount of the ionizable lipid is from about 29-44 mol % of the lipid component; the amount of the neutral lipid is from about 11-28 mol % of the lipid component; the amount of the helper lipid is from about 28-55 mol % of the lipid component; and the amount of the PEG lipid is from about 2.3-3.5 mol % of the lipid component. In certain embodiments, the amount of the ionizable lipid is from about 29-38 mol % of the lipid component; the amount of the neutral lipid is from about 11-20 mol % of the lipid component; the amount of the helper lipid is from about 43-55 mol % of the lipid component; and the amount of the PEG lipid is from about 2.3-2.7 mol % of the lipid component. In certain embodiments, the amount of the ionizable lipid is from about 25-34 mol % of the lipid component; the amount of the neutral lipid is from about 10-20 mol % of the lipid component; the amount of the helper lipid is from about 45-65 mol % of the lipid component; and the amount of the PEG lipid is from about 2.5-3.5 mol % of the lipid component. In certain embodiments, the ionizable lipid is about 30-43 mol % of the lipid component; the amount of the neutral lipid is about 10-17 mol % of the lipid component; the amount of the helper lipid is about 43.5-56 mol % of the lipid component; and the amount of the PEG lipid is about 1.5-3 mol % of the lipid component. In certain embodiments, the ionizable lipid is about 33 mol % of the lipid component; the amount of the neutral lipid is about 15 mol % of the lipid component; the amount of the helper lipid is about 49 mol % of the lipid component; and the amount of the PEG lipid is about 3 mol % of the lipid component. In certain embodiments, the amount of the ionizable lipid is about 32.9 mol % of the lipid component; the amount of the neutral lipid is about 15.2 mol % of the lipid component; the amount of the helper lipid is about 49.2 mol % of the lipid component; and the amount of the PEG lipid is about 2.7 mol % of the lipid component.

In certain embodiments, the amount of the ionizable lipid (e.g., Lipid A or one of its analogs) is about 20-50 mol %, about 25-34 mol %, about 25-38 mol %, about 25-45 mol %, about 29-38 mol %, about 29-43 mol %, about 29-34 mol %, about 30-34 mol %, about 30-38 mol %, about 30-43 mol %, about 30-43 mol %, or about 33 mol %. In certain embodiments, the amount of the neutral lipid is about 10-30 mol %, about 11-30 mol %, about 11-20 mol %, about 13-17 mol %, or about 15 mol %. In certain embodiments, the amount of the helper lipid is about 35-50 mol %, about 35-65 mol %, about 35-55 mol %, about 38-50 mol %, about 38-55 mol %, about 38-65 mol %, about 40-50 mol %, about 40-65 mol %, about 43-65 mol %, about 43-55 mol %, or about 49 mol %. In certain embodiments, the amount of the PEG lipid is about 1.5-3.5 mol %, about 2.0-2.7 mol %, about 2.0-3.5 mol %, about 2.3-3.5 mol %, about 2.3-2.7 mol %, about 2.5-3.5 mol %, about 2.5-2.7 mol %, about 2.9-3.5 mol %, or about 2.7 mol %.

Other embodiments of the present disclosure provide LNP compositions, for example, LNP compositions comprising an ionizable lipid (e.g., Lipid D or one of its analogs), a helper lipid, a helper lipid, and a PEG lipid, described according to the respective molar ratios of the component lipids in the formulation. In certain embodiments, the amount of the ionizable lipid is from about 25 mol % to about 50 mol %; the amount of the neutral lipid is from about 7 mol % to about 25 mol %; the amount of the helper lipid is from about 39 mol % to about 65 mol %; and the amount of the PEG lipid is from about 0.5 mol % to about 1.8 mol %. In certain embodiments, the amount of the ionizable lipid is from about 27-40 mol % of the lipid component; the amount of the neutral lipid is from about 10-20 mol % of the lipid component; the amount of the helper lipid is from about 50-60 mol % of the lipid component; and the amount of the PEG lipid is from about 0.9-1.6 mol % of the lipid component. In certain embodiments, the amount of the ionizable lipid is from about 30-45 mol % of the lipid component; the amount of the neutral lipid is from about 10-15 mol % of the lipid component; the amount of the helper lipid is from about 39-59 mol % of the lipid component; and the amount of the PEG lipid is from about 1-1.5 mol % of the lipid component. In certain embodiments, the amount of the ionizable lipid is from about 30-45 mol % of the lipid component; the amount of the neutral lipid is from about 10-15 mol % of the lipid component; the amount of the helper lipid is from about 39-59 mol % of the lipid component; and the amount of the PEG lipid is from about 1-1.5 mol % of the lipid component. In certain embodiments, the ionizable lipid is about 30 mol % of the lipid component; the amount of the neutral lipid is about 10 mol % of the lipid component; the amount of the helper lipid is about 59 mol % of the lipid component; and the amount of the PEG lipid is about 1-1.5 mol % of the lipid component. In certain embodiments, the amount of the ionizable lipid is about 40 mol % of the lipid component; the amount of the neutral lipid is about 15 mol % of the lipid component; the amount of the helper lipid is about 43.5 mol % of the lipid component; and the amount of the PEG lipid is about 1.5 mol % of the lipid component. In certain embodiments, the amount of the ionizable lipid is about 50 mol % of the lipid component; the amount of the neutral lipid is about 10 mol % of the lipid component; the amount of the helper lipid is about 39 mol % of the lipid component; and the amount of the PEG lipid is about 1 mol % of the lipid component.

In certain embodiments, the amount of the ionizable lipid (e.g., Lipid D or one of its analogs) is about 20-55 mol %, about 20-45 mol %, about 20-40 mol %, about 27-40 mol %, about 27-45 mol %, about 27-55 mol %, about 30-40 mol %, about 30-45 mol %, about 30-55 mol %, about 30 mol %, about 40 mol %, or about 50 mol %. In certain embodiments, the amount of the neutral lipid is about 7-25 mol %, about 10-25 mol %, about 10-20 mol %, about 15-20 mol %, about 8-15 mol %, about 10-15 mol %, about 10 mol %, or about 15 mol %. In certain embodiments, the amount of the helper lipid is about 39-65 mol %, about 39-59 mol %, about 40-60 mol %, about 40-65 mol %, about 40-59 mol %, about 43-65 mol %, about 43-60 mol %, about 43-59 mol %, or about 50-65 mol %, about 50-59 mol %, about 59 mol %, or about 43.5 mol %. In certain embodiments, the amount of the PEG lipid is about 0.5-1.8 mol %, about 0.8-1.6 mol %, about 0.8-1.5 mol %, 0.9-1.8 mol %, about 0.9-1.6 mol %, about 0.9-1.5 mol %, 1-1.8 mol %, about 1-1.6 mol %, about 1-1.5 mol %, about 1 mol %, or about 1.5 mol %.

In some embodiments, the cargo includes an mRNA encoding an RNA-guided DNA-binding agent (e.g. a Cas nuclease, a Class 2 Cas nuclease, or Cas9), or a gRNA or a nucleic acid encoding a gRNA, or a combination of mRNA and gRNA. In one embodiment, a lipid nucleic acid assembly composition may comprise a Lipid A or its equivalents, or an amine lipid as provided in WO2020219876; or Lipid D or an amine lipid provided in WO2020/072605. In some aspects, the amine lipid is Lipid A, or Lipid D. In some aspects, the amine lipid is a Lipid A equivalent, e.g. an analog of Lipid A, or an amine lipid provided in WO2020/219876. In certain aspects, the amine lipid is an acetal analog of Lipid A, optionally, an amine lipid provided in WO2020/219876. In some aspects, the amine lipid is a Lipid D or an amine lipid found in in W2020072605. In various embodiments, a lipid nucleic acid assembly composition comprises an amine lipid, a neutral lipid, a helper lipid, and a PEG lipid. In some embodiments, the helper lipid is cholesterol. In some embodiments, the neutral lipid is DSPC. In specific embodiments, PEG lipid is PEG2k-DMG. In some embodiments, a lipid nucleic acid assembly composition may comprise a Lipid A, a helper lipid, a neutral lipid, and a PEG lipid. In some embodiments, a lipid nucleic acid assembly composition comprises an amine lipid, DSPC, cholesterol, and a PEG lipid. In some embodiments, the lipid nucleic acid assembly composition comprises a PEG lipid comprising DMG. In some embodiments, the amine lipid is selected from Lipid A, and an equivalent of Lipid A, including an acetal analog of Lipid A, or an amine lipid provided in WO2020/219876; or Lipid D or an amine lipid provided in WO2020/072605. In additional embodiments, a lipid nucleic acid assembly composition comprises Lipid A, cholesterol, DSPC, and PEG2k-DMG. In additional embodiments, a lipid nucleic acid assembly composition comprises Lipid D, cholesterol, DSPC, and PEG2k-DMG.

Embodiments of the present disclosure also provide lipid compositions described according to the molar ratio between the positively charged amine groups of the amine lipid (N) and the negatively charged phosphate groups (P) of the nucleic acid to be encapsulated. This may be mathematically represented by the equation N/P. In some embodiments, a lipid nucleic acid assembly composition may comprise a lipid component that comprises an amine lipid, a helper lipid, a neutral lipid, and a helper lipid; and a nucleic acid component, wherein the N/P ratio is about 3 to 10. In some embodiments, the LNPs comprise molar ratios of an amine lipid to RNA/DNA phosphate (N:P) of about 4.5, 5.0, 5.5, 6.0, or 6.5. In some embodiments, a lipid nucleic acid assembly composition may comprise a lipid component that comprises an amine lipid, a helper lipid, a neutral lipid, and a helper lipid; and an RNA component, wherein the N/P ratio is about 3 to 10. In one embodiment, the N/P ratio may about 5-7. In one embodiment, the N/P ratio may about 4.5-8. In one embodiment, the N/P ratio may about 6. In one embodiment, the N/P ratio may be 6±1. In one embodiment, the N/P ratio may about 6±0.5. In some embodiments, the N/P ratio will be ±30%, ±25%, ±20%, ±15%, ±10%, ±5%, or ±2.5% of the target N/P ratio. In some embodiments, lipid nucleic acid assembly inter-lot variability will be less than 15%, less than 10% or less than 5%.

In some embodiments, the lipid nucleic acid assembly comprises an RNA component, which may comprise an mRNA, such as an mRNA encoding a Cas nuclease. In one embodiment, RNA component may comprise a Cas9 mRNA. In some compositions comprising an mRNA encoding a Cas nuclease, the lipid nucleic acid assembly further comprises a gRNA nucleic acid, such as a gRNA. In some embodiments, the RNA component comprises a Cas nuclease mRNA and a gRNA. In some embodiments, the RNA component comprises a Class 2 Cas nuclease mRNA and a gRNA.

In some embodiments, a lipid nucleic acid assembly composition may comprise an mRNA encoding a Cas nuclease such as a Class 2 Cas nuclease, an amine lipid, a helper lipid, a neutral lipid, and a PEG lipid. In certain lipid nucleic acid assembly compositions comprising an mRNA encoding a Cas nuclease such as a Class 2 Cas nuclease, the helper lipid is cholesterol. In other compositions comprising an mRNA encoding a Cas nuclease such as a Class 2 Cas nuclease, the neutral lipid is DSPC. In additional embodiments comprising an mRNA encoding a Cas nuclease such as a Class 2 Cas nuclease, the PEG lipid is PEG2k-DMG or PEG2k-C11. In specific compositions comprising an mRNA encoding a Cas nuclease such as a Class 2 Cas nuclease, the amine lipid is selected from Lipid A and its equivalents, such as an acetal analog of Lipid A, or amine lipids provided in WO2020/219876; or Lipid D and amine lipids provided in WO2020/072605.

In some embodiments, a lipid nucleic acid assembly composition may comprise a gRNA. In some embodiments, a lipid nucleic acid assembly composition may comprise an amine lipid, a gRNA, a helper lipid, a neutral lipid, and a PEG lipid. In certain lipid nucleic acid assembly compositions comprising a gRNA, the helper lipid is cholesterol. In some compositions comprising a gRNA, the neutral lipid is DSPC. In additional embodiments comprising a gRNA, the PEG lipid is PEG2k-DMG or PEG2k-C11. In some embodiments, the amine lipid is selected from Lipid A and its equivalents, such as an acetal analog of Lipid A, or amine lipids provided in WO2020/219876 and their equivalents; or Lipid D and amine lipids provided in WO2020/072605 and their equivalents.

In one embodiment, a lipid nucleic acid assembly composition may comprise an sgRNA. In one embodiment, a lipid nucleic acid assembly composition may comprise a Cas9 sgRNA. In one embodiment, a lipid nucleic acid assembly composition may comprise a Cpf1 sgRNA. In some compositions comprising an sgRNA, the lipid nucleic acid assembly includes an amine lipid, a helper lipid, a neutral lipid, and a PEG lipid. In certain compositions comprising an sgRNA, the helper lipid is cholesterol. In other compositions comprising an sgRNA, the neutral lipid is DSPC. In additional embodiments comprising an sgRNA, the PEG lipid is PEG2k-DMG or PEG2k-C11. In some embodiments, the amine lipid is selected from Lipid A and its equivalents, such as acetal analogs of Lipid A, or amine lipids provided in WO2020/219876; or Lipid D and amine lipids provided in WO2020/072605.

In some embodiments, a lipid nucleic acid assembly composition comprises an mRNA encoding a Cas nuclease and a gRNA, which may be an sgRNA. In one embodiment, a lipid nucleic acid assembly composition may comprise an amine lipid, an mRNA encoding a Cas nuclease, a gRNA, a helper lipid, a neutral lipid, and a PEG lipid. In certain compositions comprising an mRNA encoding a Cas nuclease and a gRNA, the helper lipid is cholesterol. In some compositions comprising an mRNA encoding a Cas nuclease and a gRNA, the neutral lipid is DSPC. In additional embodiments comprising an mRNA encoding a Cas nuclease and a gRNA, the PEG lipid is PEG2k-DMG or PEG2k-C11. In some embodiments, the amine lipid is selected from Lipid A and its equivalents, such as acetal analogs of Lipid A, or amine lipids provided in WO2020/219876; or Lipid D and amine lipids provided in WO2020/072605.

In some embodiments, the lipid nucleic acid assembly compositions include a Cas nuclease mRNA, such as a Class 2 Cas mRNA and at least one gRNA. In some embodiments, the lipid nucleic acid assembly composition includes a ratio of gRNA to Cas nuclease mRNA, such as Class 2 Cas nuclease mRNA from about 25:1 to about 1:25 wt/wt. In some embodiments, the lipid nucleic acid assembly formulation includes a ratio of gRNA to Cas nuclease mRNA, such as Class 2 Cas nuclease mRNA from about 10:1 to about 1:10. In some embodiments, the lipid nucleic acid assembly formulation includes a ratio of gRNA to Cas nuclease mRNA, such as Class 2 Cas nuclease mRNA from about 8:1 to about 1:8. As measured herein, the ratios are by weight. In some embodiments, the lipid nucleic acid assembly formulation includes a ratio of gRNA to Cas nuclease mRNA, such as Class 2 Cas mRNA from about 5:1 to about 1:5. In some embodiments, ratio range is about 3:1 to 1:3, about 2:1 to 1:2, about 5:1 to 1:2, about 5:1 to 1:1, about 3:1 to 1:2, about 3:1 to 1:1, about 3:1, about 2:1 to 1:1. In some embodiments, the gRNA to mRNA ratio is about 3:1 or about 2:1. In some embodiments the ratio of gRNA to Cas nuclease mRNA, such as Class 2 Cas nuclease is about 1:1. In some embodiments the ratio of gRNA to Cas nuclease mRNA, such as Class 2 Cas nuclease is about 1:2. The ratio may be about 25:1, 10:1, 5:1, 3:1, 2:1, 1:1, 1:2, 1:3, 1:5, 1:10, or 1:25.

The lipid nucleic acid assembly compositions disclosed herein may include a template nucleic acid. The template nucleic acid may be co-formulated with an mRNA encoding a Cas nuclease, such as a Class 2 Cas nuclease mRNA. In some embodiments, the template nucleic acid may be co-formulated with a guide RNA. In some embodiments, the template nucleic acid may be co-formulated with both an mRNA encoding a Cas nuclease and a guide RNA. In some embodiments, the template nucleic acid may be formulated separately from an mRNA encoding a Cas nuclease or a guide RNA. The template nucleic acid may be delivered with, or separately from the lipid nucleic acid assembly compositions. In some embodiments, the template nucleic acid may be single- or double-stranded, depending on the desired repair mechanism. The template may have regions of homology to the target DNA, or to sequences adjacent to the target DNA.

In some embodiments, a lipid nucleic acid assemblies are formed by mixing an aqueous RNA solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer, e.g., for in vivo administration of lipid nucleic acid assemblies, may be used. In some embodiments, a buffer is used to maintain the pH of the composition comprising lipid nucleic acid assemblies at or above pH 6.5. In some embodiments, a buffer is used to maintain the pH of the composition comprising lipid nucleic acid assemblies at or above pH 7.0. In some embodiments, the composition has a pH ranging from about 7.2 to about 7.7. In additional embodiments, the composition has a pH ranging from about 7.3 to about 7.7 or ranging from about 7.4 to about 7.6. In further embodiments, the composition has a pH of about 7.2, 7.3, 7.4, 7.5, 7.6, or 7.7. The pH of a composition may be measured with a micro pH probe. In some embodiments, a cryoprotectant is included in the composition. Non-limiting examples of cryoprotectants include sucrose, trehalose, glycerol, DMSO, and ethylene glycol. Exemplary compositions may include up to 10% cryoprotectant, such as, for example, sucrose. In some embodiments, the lipid nucleic acid assembly composition may include about 1, 2, 3, 4, 5, 6, 7, 8, 9, or 10% cryoprotectant. In some embodiments, the lipid nucleic acid assembly composition may include about 1, 2, 3, 4, 5, 6, 7, 8, 9, or 10% sucrose. In some embodiments, the lipid nucleic acid assembly composition may include a buffer. In some embodiments, the buffer may comprise a phosphate buffer (PBS), a Tris buffer, a citrate buffer, and mixtures thereof. In some exemplary embodiments, the buffer comprises NaCl. In some embodiments, NaCl is omitted. Exemplary amounts of NaCl may range from about 20 mM to about 45 mM. Exemplary amounts of NaCl may range from about 40 mM to about 50 mM. In some embodiments, the amount of NaCl is about 45 mM. In some embodiments, the buffer is a Tris buffer. Exemplary amounts of Tris may range from about 20 mM to about 60 mM. Exemplary amounts of Tris may range from about 40 mM to about 60 mM. In some embodiments, the amount of Tris is about 50 mM. In some embodiments, the buffer comprises NaCl and Tris. Certain exemplary embodiments of the lipid nucleic acid assembly compositions contain 5% sucrose and 45 mM NaCl in Tris buffer. In other exemplary embodiments, compositions contain sucrose in an amount of about 5% w/v, about 45 mM NaCl, and about 50 mM Tris at pH 7.5. The salt, buffer, and cryoprotectant amounts may be varied such that the osmolality of the overall formulation is maintained. For example, the final osmolality may be maintained at less than 450 mOsm/L. In further embodiments, the osmolality is between 350 and 250 mOsm/L. Certain embodiments have a final osmolality of 300+/−20 mOsm/L.

In some embodiments, microfluidic mixing, T-mixing, or cross-mixing is used. In certain aspects, flow rates, junction size, junction geometry, junction shape, tube diameter, solutions, and/or RNA and lipid concentrations may be varied. Lipid nucleic acid assemblies or lipid nucleic acid assembly compositions may be concentrated or purified, e.g., via dialysis, tangential flow filtration, or chromatography. The lipid nucleic acid assemblies may be stored as a suspension, an emulsion, or a lyophilized powder, for example. In some embodiments, a lipid nucleic acid assembly composition is stored at 2-8° C., in certain aspects, the lipid nucleic acid assembly compositions are stored at room temperature. In additional embodiments, a lipid nucleic acid assembly composition is stored frozen, for example at −20° C. or −80° C. In other embodiments, a lipid nucleic acid assembly composition is stored at a temperature ranging from about 0° C. to about −80° C. Frozen lipid nucleic acid assembly compositions may be thawed before use, for example on ice, at 4° C., at room temperature, or at 25° C. Frozen lipid nucleic acid assembly compositions may be maintained at various temperatures, for example on ice, at 4° C., at room temperature, at 25° C., or at 37° C.

In some embodiments, the concentration of the LNPs in the LNP composition is about 1-10 ug/mL, about 2-10 ug/mL, about 2.5-10 ug/mL, about 1-5 ug/mL, about 2-5 ug/mL, about 2.5-5 ug/mL, about 0.04 ug/mL, about 0.08 ug/mL, about 0.16 ug/mL, about 0.25 ug/mL, about 0.63 ug/mL, about 1.25 ug/mL, about 2.5 ug/mL, or about 5 ug/mL.

In some embodiments, the lipid nucleic acid assembly composition comprises a stealth lipid, optionally wherein:

(i) the lipid nucleic acid assembly composition comprises a lipid component and the lipid component comprises: about 50-60 mol % amine lipid such as Lipid A or Lipid D, about 8-10 mol % neutral lipid; and about 2.5-4 mol % stealth lipid (e.g., a PEG lipid), wherein the remainder of the lipid component is helper lipid, and wherein the N/P ratio of the lipid nucleic acid assembly composition is about 6;
(ii) the lipid nucleic acid assembly composition comprises about 50-60 mol % amine lipid such as Lipid A or Lipid D; about 27-39.5 mol % helper lipid; about 8-10 mol % neutral lipid; and about 2.5-4 mol % stealth lipid (e.g., a PEG lipid), wherein the N/P ratio of the lipid nucleic acid assembly composition is about 5-7 (e.g., about 6);
(iii) the lipid nucleic acid assembly composition comprises a lipid component and the lipid component comprises: about 50-60 mol % amine lipid such as Lipid A or Lipid D; about 5-15 mol % neutral lipid; and about 2.5-4 mol % stealth lipid (e.g., a PEG lipid), wherein the remainder of the lipid component is helper lipid, and wherein the N/P ratio of the lipid nucleic acid assembly composition is about 3-10;
(iv) the lipid nucleic acid assembly composition comprises a lipid component and the lipid component comprises: about 40-60 mol % amine lipid such as Lipid A or Lipid D; about 5-15 mol % neutral lipid; and about 2.5-4 mol % stealth lipid (e.g., a PEG lipid), wherein the remainder of the lipid component is helper lipid, and wherein the N/P ratio of the lipid nucleic acid assembly composition is about 6;
(v) the lipid nucleic acid assembly composition comprises a lipid component and the lipid component comprises: about 50-60 mol % amine lipid such as Lipid A or Lipid D; about 5-15 mol % neutral lipid; and about 1.5-10 mol % stealth lipid (e.g., a PEG lipid), wherein the remainder of the lipid component is helper lipid, and wherein the N/P ratio of the lipid nucleic acid assembly composition is about 6;
(vi) the lipid nucleic acid assembly composition comprises a lipid component and the lipid component comprises: about 40-60 mol % amine lipid such as Lipid A or Lipid D; about 0-10 mol % neutral lipid; and about 1.5-10 mol % stealth lipid (e.g., a PEG lipid), wherein the remainder of the lipid component is helper lipid, and wherein the N/P ratio of the lipid nucleic acid assembly composition is about 3-10;
(vii) the lipid nucleic acid assembly composition comprises a lipid component and the lipid component comprises: about 40-60 mol % amine lipid such as Lipid A or Lipid D; less than about 1 mol % neutral lipid; and about 1.5-10 mol % stealth lipid (e.g., a PEG lipid), wherein the remainder of the lipid component is helper lipid, and wherein the N/P ratio of the lipid nucleic acid assembly composition is about 3-10;
(viii) the lipid nucleic acid assembly composition comprises a lipid component and the lipid component comprises: about 40-60 mol % amine lipid such as Lipid A or Lipid D; and about 1.5-10 mol % stealth lipid (e.g., a PEG lipid), wherein the remainder of the lipid component is helper lipid, wherein the N/P ratio of the LNP composition is about 3-10, and wherein the lipid nucleic acid assembly composition is essentially free of or free of neutral phospholipid; or
(ix) the lipid nucleic acid assembly composition comprises a lipid component and the lipid component comprises: about 50-60 mol % amine lipid such as Lipid A or Lipid D; about 8-10 mol-% neutral lipid; and about 2.5-4 mol % stealth lipid (e.g., a PEG lipid), wherein the remainder of the lipid component is helper lipid, and wherein the N/P ratio of the lipid nucleic acid assembly composition is about 3-7.

In some embodiments, the lipid nucleic acid assembly composition comprises a lipid component and the lipid component comprises: about 50 mol % amine lipid such as Lipid A or Lipid D; about 9 mol % neutral lipid such as DSPC; about 3 mol % of stealth lipid such as a PEG lipid, such as PEG2k-DMG, and the remainder of the lipid component is helper lipid such as cholesterol wherein the N/P ratio of the lipid nucleic acid assembly composition is about 6.

In some embodiments, the lipid nucleic acid assembly composition comprises a lipid component and the lipid component comprises: about 50 mol % Lipid A; about 9 mol % DSPC; about 3 mol % of PEG2k-DMG, and the remainder of the lipid component is cholesterol wherein the N/P ratio of the lipid nucleic acid assembly composition is about 6.

In some embodiments, the lipid nucleic acid assembly composition comprises a lipid component and the lipid component comprises: about 35 mol % Lipid A; about 15 mol % neutral lipid; about 47.5 mol % helper lipid; and about 2.5 mol % stealth lipid (e.g., PEG lipid), and wherein the N/P ratio of the LNP composition is about 3-7.

In some embodiments, the lipid nucleic acid assembly composition comprises a lipid component and the lipid component comprises: about 35 mol % Lipid D; about 15 mol % neutral lipid; about 47.5 mol % helper lipid; and about 2.5 mol % stealth lipid (e.g., PEG lipid), and wherein the N/P ratio of the LNP composition is about 3-7.

In some embodiments, the lipid nucleic acid assembly composition comprises a lipid component and the lipid component comprises: about 25-45 mol % amine lipid, such as Lipid A; about 10-30 mol % neutral lipid; about 25-65 mol % helper lipid; and about 1.5-3.5 mol % stealth lipid (e.g., PEG lipid), and wherein the N/P ratio of the LNP composition is about 3-7.

In some embodiments, the lipid nucleic acid assembly composition comprises a lipid component, wherein:
a. the amount of the amine lipid is about 29-44 mol % of the lipid component; the amount of the neutral lipid is about 11-28 mol % of the lipid component; the amount of the helper lipid is about 28-55 mol % of the lipid component; and the amount of the PEG lipid is about 2.3-3.5 mol % of the lipid component
b. the amount of the amine lipid is about 29-38 mol % of the lipid component; the amount of the neutral lipid is about 11-20 mol % of the lipid component; the amount of the helper lipid is about 43-55 mol % of the lipid component; and the amount of the PEG lipid is about 2.3-2.7 mol % of the lipid component;
c. the amount of the amine lipid is about 25-34 mol % of the lipid component; the amount of the neutral lipid is about 10-20 mol % of the lipid component; the amount of the helper lipid is about 45-65 mol % of the lipid component; and the amount of the PEG lipid is about 2.5-3.5 mol % of the lipid component; or
d. the amount of the amine lipid is about 30-43 mol % of the lipid component; the amount of the neutral lipid is about 10-17 mol % of the lipid component; the amount of the helper lipid is about 43.5-56 mol % of the lipid component; and the amount of the PEG lipid is about 1.5-3 mol % of the lipid component.

In some embodiments, the lipid nucleic acid assembly composition comprises a lipid component and the lipid component comprises: about 25-50 mol % amine lipid, such as Lipid D; about 7-25 mol % neutral lipid; about 39-65 mol % helper lipid; and about 0.5-1.8 mol % stealth lipid (e.g., PEG lipid), and wherein the N/P ratio of the LNP composition is about 3-7.

In some embodiments, the lipid nucleic acid assembly composition comprises a lipid component wherein the amount of the amine lipid is about 30-45 mol % of the lipid component; or about 30-40 mol % of the lipid component; optionally about 30 mol %, 40 mol %, or 50 mol % of the lipid component. In some embodiments, the lipid nucleic acid assembly composition comprises a lipid component wherein the amount of the neutral lipid is about 10-20 mol % of the lipid component; or about 10-15 mol % of the lipid component; optionally about 10 mol % or 15 mol % of the lipid component. In some embodiments, the lipid nucleic acid assembly composition comprises a lipid component wherein the amount of the helper lipid is about 50-60 mol % of the lipid component; about 39-59 mol % of the lipid component; or about 43.5-59 mol % of the lipid component; optionally about 59 mol % of the lipid component; about 43.5 mol % of the lipid component; or about 39 mol % of the lipid component. In some embodiments, the lipid nucleic acid assembly composition comprises a lipid component wherein the amount of the PEG lipid is about 0.9-1.6 mol % of the lipid component; or about 1-1.5 mol % of the lipid component; optionally about 1 mol % of the lipid component or about 1.5 mol % of the lipid component

In some embodiments, the lipid nucleic acid assembly composition comprises a lipid component, wherein:
a. the amount of the ionizable lipid is about 27-40 mol % of the lipid component; the amount of the neutral lipid is about 10-20 mol % of the lipid component; the amount of the helper lipid is about 50-60 mol % of the lipid component; and the amount of the PEG lipid is about 0.9-1.6 mol % of the lipid component;
b. the amount of the ionizable lipid is from about 30-45 mol % of the lipid component; the amount of the neutral lipid is from about 10-15 mol % of the lipid component; the amount of the helper lipid is from about 39-59 mol % of the lipid component; and the amount of the PEG lipid is from about 1-1.5 mol % of the lipid component;
c. the amount of the ionizable lipid is about 30 mol % of the lipid component; the amount of the neutral lipid is about 10 mol % of the lipid component; the amount of the helper lipid is about 59 mol % of the lipid component; and the amount of the PEG lipid is about 1-1.5 mol % of the lipid component;
d. the amount of the ionizable lipid is about 40 mol % of the lipid component; the amount of the neutral lipid is about 15 mol % of the lipid component; the amount of the helper lipid is about 43.5 mol % of the lipid component; and the amount of the PEG lipid is about 1.5 mol % of the lipid component; or
e. the amount of the ionizable lipid is about 50 mol % of the lipid component; the amount of the neutral lipid is about 10 mol % of the lipid component; the amount of the helper lipid is about 39 mol % of the lipid component; and the amount of the PEG lipid is about 1 mol % of the lipid component.

In some embodiments, the LNP has a diameter of about 1-250 nm, 10-200 nm, about 20-150 nm, about 50-150 nm, about 50-100 nm, about 50-120 nm, about 60-100 nm, about 75-150 nm, about 75-120 nm, or about 75-100 nm. In some embodiments, the LNP has a diameter of less than 100 nm. In some embodiments, the LNP composition comprises a population of the LNP with an average diameter of about 10-200 nm, about 20-150 nm, about 50-150 nm, about 50-100 nm, about 50-120 nm, about 60-100 nm, about 75-150 nm, about 75-120 nm, or about 75-100 nm. In some embodiments, the LNP has an average diameter of less than 100 nm.

In some embodiments, the lipid nucleic acid assembly composition comprises: about 40-60 mol-% amine lipid; about 5-15 mol-% neutral lipid; and about 1.5-10 mol-% PEG lipid, wherein the remainder of the lipid component is helper lipid, and wherein the N/P ratio of the LNP composition is about 3-10. In some embodiments, the lipid nucleic acid assembly composition comprises: about 50-60 mol-% amine lipid; about 8-10 mol-% neutral lipid; and about 2.5-4 mol-% PEG lipid, wherein the remainder of the lipid component is helper lipid, and wherein the N/P ratio of the LNP composition is about 3-8. In some embodiments, the lipid nucleic acid assembly composition comprises: about 50-60 mol-% amine lipid; about 5-15 mol-% DSPC; and about 2.5-4 mol-% PEG lipid, wherein the remainder of the lipid component is cholesterol, and wherein the N/P ratio of the LNP composition is 3-8±0.2.

In embodiments, the average diameter is a Z-average diameter. In certain embodiments, the Z-average diameter is measured by dynamic light scattering (DLS) using methods known in the art. For example, average particle size and polydispersity can be measured by dynamic light scattering (DLS) using a Malvern Zetasizer DLS instrument. LNP samples are diluted with PBS buffer prior to being measured by DLS. Z-average diameter and number average diameter along with a polydispersity index (pdi) can be determined. The Z average is the intensity weighted mean hydrodynamic size of the ensemble collection of particles. The number average is the particle number weighted mean hydrodynamic size of the ensemble collection of particles. A Malvern Zetasizer instrument can also be used to measure the zeta potential of the LNP using methods known in the art.

D. DNA-Dependent Protein Kinase Inhibitors

DNA-dependent protein kinase (DNA-PK) is a nuclear serine/threonine kinase that has been shown to be essential in DNA double stranded break repair machinery. In mammals, the predominant pathway for repair of double stranded DNA breaks is the non-homologous end joining (NHEJ) pathway which is functional regardless of the phase of the cell cycle and acts by removing non-ligatable ends and ligating ends of double strand breaks. DNA-PK inhibitors (DNA-PKi) are a structurally diverse class of inhibitors of DNA-PK, and the NHEJ pathway. Exemplary DNA-PKi are provided, for example, in WO03024949, WO2014159690A1, and WO2018114999.

DNA-dependent protein kinase (DNA-PK) is a nuclear serine/threonine kinase that has been shown to be essential in DNA double stranded break repair machinery. In mammals, the predominant pathway for repair of double stranded DNA breaks is the non-homologous end joining (NHEJ) pathway which is functional regardless of the phase of the cell cycle and acts by removing non-ligatable ends and ligating ends of double strand breaks. DNA-PK inhibitors (DNA-PKi) are a structurally diverse class of inhibitors of DNA-PK, and the NHEJ pathway. Exemplary DNA-PKi are provided, for example, in WO03024949, WO2014159690A1, and WO2018114999.

In preferred embodiments, the disclosure relates to a DNAPKI Compound 1 that is

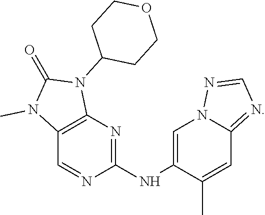 [SMILES: Cc1cc2ncnn2cc1Nc1ncc2c(n1)n(C1CCOCC1)c(=O)n2C]

In preferred embodiments, the disclosure relates to a DNAPKI Compound 3 that is

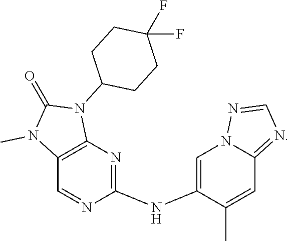 [SMILES: Cc1cc2ncnn2cc1Nc1ncc2c(n1)n(C1CCC(F)(F)CC1)c(=O)n2C]

In preferred embodiments, the disclosure relates to a DNAPKI Compound 4 that is

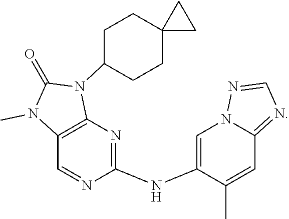 [SMILES: Cc1cc2ncnn2cc1Nc1ncc2c(n1)n(C1CCC3(CC1)CC3)c(=O)n2C]

In certain embodiments, the disclosure relates to any of the compositions described herein, wherein the concentration of the DNAPKI in the composition is about 1 μM or less, for example, about 0.25 μM or less, such as about 0.1-1 preferably about 0.1-0.5 μM.

In some embodiments, the DNAPKI is formed according to the methods set forth in WO2018114999, which is incorporated by reference.

Exemplary DNA-PKi include, but are not limited to, Compound 1, Compound 3 and Compound 4. In some embodiments, the DNAPKi is Compound 1. In some embodiments, the DNAPKI is Compound 3. In some embodiments, the DNAPKi is Compound 4.

1. Synthesis of DNA-Dependent Protein Kinase Inhibitors

a) Compound 1

### Intermediate 1a: (E)-N,N-dimethyl-N′-(4-methyl-5-nitropyridin-2-yl)formimidamide

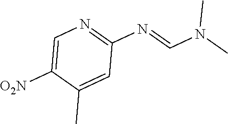

To a solution of 4-methyl-5-nitro-pyridin-2-amine (5 g, 1.0 equiv.) in toluene (0.3 M) was added DMF-DMA (3.0 equiv.). The mixture was stirred at 110° C. for 2 h. The reaction mixture was concentrated under reduced pressure to give a residue and purified by column chromatography to afford product as a yellow solid (59%). 1H NMR (400 MHz, (CD3)2SO) δ 8.82 (s, 1H), 8.63 (s, 1H), 6.74 (s, 1H), 3.21 (m, 6H).

### Intermediate 1b: (E)-N-hydroxy-N′-(4-methyl-5-nitropyridin-2-yl)formimidamide

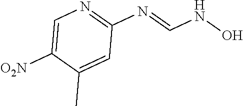

To a solution of Intermediate 1a (4 g, 1.0 equiv.) in MeOH (0.2 M) was added NH2OH·HCl (2.0 equiv.). The reaction mixture was stirred at 80° C. for 1 h. The reaction mixture was filtered, and the filtrate was concentrated under reduced pressure to give a residue. The residue was partitioned between H2O and EtOAc, followed by 2× extraction with EtOAc. The organic phases were concentrated under reduced pressure to give a residue and purified by column chromatography to afford product as a white solid (66%). 1H NMR (400 MHz, (CD3)2SO) δ 10.52 (d, J=3.8 Hz, 1H), 10.08 (dd, J=9.9, 3.7 Hz, 1H), 8.84 (d, J=3.8 Hz, 1H), 7.85 (dd, J=9.7, 3.8 Hz, 1H), 7.01 (d, J=3.9 Hz, 1H), 3.36 (s, 3H).

### Intermediate 1c: 7-methyl-6-nitro-[1,2,4]triazolo[1,5-a]pyridine

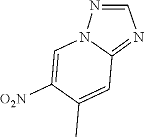

To a solution of Intermediate 1b (2.5 g, 1.0 equiv.) in THF (0.4 M) was added trifluoroacetic anhydride (1.0 equiv.) at 0° C. The mixture was stirred at 25° C. for 18 h. The reaction mixture was filtered, and the filtrate was concentrated under reduced pressure to give a residue. The residue was purified by column chromatography to afford product as a white solid (44%). 1H NMR (400 MHz, CDCl₃) δ 9.53 (s, 1H), 8.49 (s, 1H), 7.69 (s, 1H), 2.78 (d, J=1.0 Hz, 3H).

### Intermediate 1d: 7-methyl-[1,2,4]triazolo[1,5-a]pyridin-6-amine

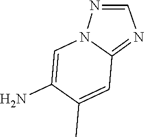

To a mixture of Pd/C (10% w/w, 0.2 equiv.) in EtOH (0.1 M) was added Intermediate 1c (1.0 equiv. and ammonium formate (5.0 equiv.). The mixture was heated at 105° C. for 2 h. The reaction mixture was filtered, and the filtrate was concentrated under reduced pressure to give a residue. The residue was purified by column chromatography to afford product as a pale brown solid. 1H NMR (400 MHz, (CD3)2SO) δ 8.41 (s, 2H), 8.07 (d, J=9.0 Hz, 2H), 7.43 (s, 1H), 2.22 (s, 3H).

### Intermediate 1e: ethyl 2-chloro-4-((tetrahydro-2H-pyran-4-yl)amino)pyrimidine-5-carboxylate

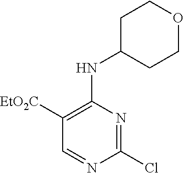 [SMILES: CCOC(=O)c1cnc(Cl)nc1NC1CCOCC1]

To a solution of tetrahydropyran-4-amine (5 g, 1.0 equiv.) and ethyl 2,4-dichloropyrimidine-5-carboxylate (1.0 equiv.) in MeCN (0.25-2.0 M) was added K2CO3 (1.0-3.0 equiv.). The mixture was stirred at 20-25° C. for at least 12 h. The reaction mixture was filtered, and the filtrate was concentrated under reduced pressure to give a residue. The residue was purified by column chromatography to afford product as a pale yellow solid (21%). 1H NMR (400 MHz, (CD3)2SO) δ 8.60 (s, 1H), 8.29 (d, J=7.7 Hz, 1H), 4.28 (q, J=7.1 Hz, 2H), 4.14 (dtt, J=11.3, 8.3, 4.0 Hz, 1H), 3.82 (dt, J=12.1, 3.6 Hz, 2H), 3.57 (s, 1H), 1.87-1.78 (m, 2H), 1.76-1.67 (m, 1H), 1.54 (qd, J=10.9, 4.3 Hz, 2H), 1.28 (t, J=7.1 Hz, 3H).

### Intermediate 1f: 2-chloro-4-((tetrahydro-2H-pyran-4-yl)amino)pyrimidine-5-carboxylic acid

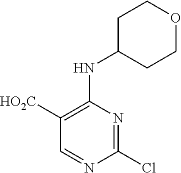 [SMILES: O=C(O)c1cnc(Cl)nc1NC1CCOCC1]

To a solution of LiOH (2.5 equiv.) in 1:1 THF/H2O (0.25-1.0 M) was added Intermediate 1e (3.0 g, 1.0 equiv.). The mixture was stirred at 25° C. for 12 h. The mixture was concentrated under reduced pressure to remove THF. The residue was adjusted pH to 2 by 2 M HCl, and the resulting precipitate was collected by filtration, washed with water, and dried under vacuum to get a residue. The residue was purified by column chromatography to afford product as a white solid (74%) or used directly as a crude product.

### Intermediate 1g: 2-chloro-9-(tetrahydro-2H-pyran-4-yl)-7,9-dihydro-8H-purin-8-one

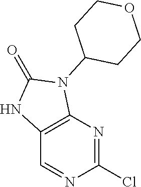 [SMILES: O=c1[nH]c2cnc(Cl)nc2n1C1CCOCC1]

To a solution of Intermediate if (2 g, 1.0 equiv.) in MeCN (0.2-0.5 M) was added Et3N (1.0 equiv.). The mixture was stirred at 25° C. for 30 min. Then DPPA (1.0 equiv.) was added to the mixture. The mixture was stirred at 100° C. for at least 7 h. The reaction mixture was poured into water, and the resulting precipitate was collected by filtration, washed with water, and dried under vacuum to get a residue. The residue was purified by column chromatography to afford product as a white solid (56%). 1H NMR (400 MHz, CDCl₃) δ 9.50 (s, 1H), 8.09 (s, 1H), 4.53 (tt, J=12.4, 4.2 Hz, 1H), 4.07 (dt, J=9.5, 4.8 Hz, 2H), 3.48 (td, J=12.1, 1.9 Hz, 2H), 2.69 (qd, J=12.5, 4.7 Hz, 2H), 1.67 (dd, J=12.1, 3.9 Hz, 2H).

### Intermediate 1h: 2-chloro-7-methyl-9-(tetrahydro-2H-pyran-4-yl)-7,9-dihydro-8H-purin-8-one

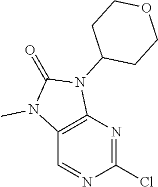 [SMILES: Cn1c(=O)n(C2CCOCC2)c2nc(Cl)ncc21]

To a mixture of Intermediate 1g (300 mg, 1.0 equiv.) and NaOH (5.0 equiv.) in 1:1 THF/H2O (0.25-1.0 M) was added iodomethane (2.0 equiv.). The reaction mixture was stirred at 25° C. for 12 h. The reaction mixture was concentrated under reduced pressure to give a residue and purified by column chromatography to afford product as a white solid (47%). 1H NMR (400 MHz, (CD3)2SO) δ 8.34 (s, 1H), 4.43 (ddt, J=12.2, 8.5, 4.2 Hz, 1H), 3.95 (dd, J=11.5, 4.6 Hz, 2H), 3.43 (td, J=12.1, 1.9 Hz, 2H), 2.45 (s, 3H), 2.40 (td, J=12.5, 4.7 Hz, 2H), 1.66 (ddd, J=12.2, 4.4, 1.9 Hz, 2H).

### Compound 1: 7-methyl-2-((7-methyl-[1,2,4]triazolo[1,5-a]pyridin-6-yl)amino)-9-(tetrahydro-2H-pyran-4-yl)-7,9-dihydro-8H-purin-8-one (Compound 1)

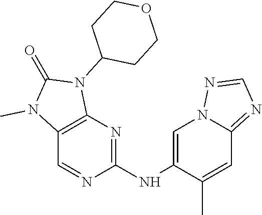 [SMILES: Cc1cc2ncnn2cc1Nc1ncc2c(n1)n(C1CCOCC1)c(=O)n2C]

A mixture of Intermediate 1h (1.3 g, 1.0 equiv.), Intermediate 1d (1.0 equiv.), Pd(dppf)Cl2 (0.1-0.2 equiv.), XantPhos (0.1-0.2 equiv.) and Cs2CO3 (2.0 equiv.) in DMF (0.05-0.3 M) was degassed and purged 3× with N2 and the mixture was stirred at 100-130° C. for at least 12 h under N2 atmosphere. The reaction mixture was then poured into water and extracted 3× with DCM. The combined organic phase was washed with brine, dried with anhydrous Na2SO4, filtered, and the filtrate was concentrated in vacuum. The residue was purified by column chromatography to afford product as a pale yellow solid. 1H NMR (400 MHz, (CD3)2SO) δ 9.13 (s, 1H), 8.69 (s, 1H), 8.39 (s, 1H), 8.10 (s, 1H), 7.72 (s, 1H), 4.50-4.36 (m, 1H), 3.98 (dd, J=11.6, 4.4 Hz, 2H), 3.44 (d, J=11.9 Hz, 2H), 3.32 (s, 3H), 2.44-2.38 (m, 3H), 1.69 (d, J=11.6 Hz, 2H). MS: 381.3 m/z [M+H].

b) Compound 3

### Intermediate 3a: ethyl 2-chloro-4-(4,4-difluorocyclohexyl)amino)pyrimidine-5-carboxylate

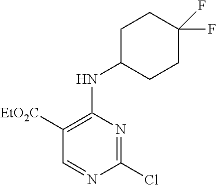 [SMILES: CCOC(=O)c1cnc(Cl)nc1NC1CCC(F)(F)CC1]

Intermediate 3a was synthesized from ethyl 2,4-dichloropyrimidine-5-carboxylate and 4,4-difluorocyclohexanamine hydrochloride using the method employed in Intermediate 1e. 1H NMR (400 MHz, (CD3)2SO) δ 8.61 (s, 1H), 8.30 (d, J=7.7 Hz, 1H), 4.29 (q, J=7.1 Hz, 2H), 4.19-4.09 (m, 1H), 2.09-1.90 (m, 6H), 1.69-1.58 (m, 2H), 1.29 (t, J=7.1 Hz, 3H).

### Intermediate 3b: 2-chloro-4-((4,4-difluorocyclohexyl)amino)pyrimidine-5-carboxylic acid

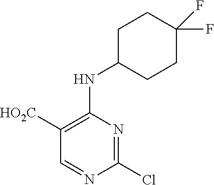 [SMILES: O=C(O)c1cnc(Cl)nc1NC1CCC(F)(F)CC1]

Intermediate 3b was synthesized (78%) from Intermediate 3a using the method employed in Intermediate 1f. 1H NMR (400 MHz, (CD3)2SO) δ 13.77 (s, 1H), 8.57 (s, 1H), 8.53 (d, J=7.8 Hz, 1H), 4.12 (d, J=10.2 Hz, 1H), 2.14-1.89 (m, 6H), 1.62 (ddt, J=17.0, 10.3, 6.0 Hz, 2H).

### Intermediate 3c: 2-chloro-9-(4,4-difluorocyclohexyl)-7,9-dihydro-8H-purin-8-one

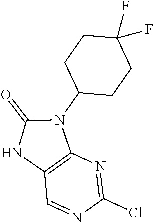 [SMILES: O=c1[nH]c2cnc(Cl)nc2n1C1CCC(F)(F)CC1]

Intermediate 3c was synthesized (56%) from Intermediate 3b using the method employed in Intermediate 1g. 1H NMR (400 MHz, (CD3)2SO) δ 11.76-11.65 (m, 1H), 8.20 (s, 1H), 4.47 (dq, J=12.6, 6.2, 4.3 Hz, 1H), 2.34-1.97 (m, 6H), 1.90 (d, J=12.9 Hz, 2H).

### Intermediate 3d: 2-chloro-9-(4,4-difluorocyclohexyl)-7-methyl-7,9-dihydro-8H-purin-8-one

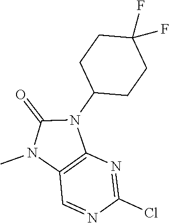 [SMILES: Cn1c(=O)n(C2CCC(F)(F)CC2)c2nc(Cl)ncc21]

To a mixture of Intermediate 3c (1.4 g, 1.0 equiv.), NaOH (5.0 equiv.) in 5:1 THF/H2O (0.3 M) was added MeI (2.0 equiv.). The mixture was stirred at 20° C. for 12 h under N2 atmosphere. The reaction mixture was concentrated under reduced pressure to give a residue and purified by column chromatography to afford product as a yellow solid (47%). 1H NMR (400 MHz, CDCl₃) δ 8.01 (s, 1H), 4.53-4.39 (m, 1H), 3.43 (s, 3H), 2.73 (qd, J=12.7, 12.1, 3.8 Hz, 2H), 2.32-2.20 (m, 2H), 2.03-1.82 (m, 4H).

### Compound 3: 9-(4,4-difluorocyclohexyl)-7-methyl-2-((7-methyl-[1,2,4]triazolo[1,5-a]pyridin-6-yl)amino)-7,9-dihydro-8H-purin-8-one (Compound 3)

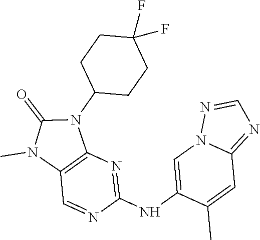 [SMILES: Cc1cc2ncnn2cc1Nc1ncc2c(n1)n(C1CCC(F)(F)CC1)c(=O)n2C]

Compound 3 was synthesized from Intermediate 1d and Intermediate 3d using the method employed for Compound 1, followed by a purification by reverse-phase HPLC. 1H NMR (400 MHz, (CD3)2SO) δ 9.03 (s, 1H), 8.66 (s, 1H), 8.38 (s, 1H), 8.10 (s, 1H), 7.71 (d, J=1.4 Hz, 1H), 4.36 (d, J=12.3 Hz, 1H), 3.31 (s, 3H), 2.38 (d, J=1.0 Hz, 3H), 2.11-1.96 (m, 4H), 1.81 (d, J=12.6 Hz, 2H). MS: 415.5 m/z [M+H].

c) Compound 4

### Intermediate 4a: 8-methylene-1,4-dioxaspiro[4.5]decane

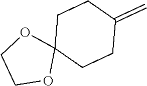

To a solution of methyl(triphenyl)phosphonium bromide (1.15 equiv.) in THF (0.6 M) was added n-BuLi (1.1 equiv.) at −78° C. dropwise, and the mixture was stirred at 0° C. for 1 h. Then, 1,4-dioxaspiro[4.5]decan-8-one (50 g, 1.0 equiv.) was added to the reaction mixture. The mixture was stirred at 25° C. for 12 h. The reaction mixture was poured into aq. NH4Cl at 0° C., diluted with H2O, and extracted 3× with EtOAc. The combined organic layers were concentrated under reduced pressure to give a residue and purified by column chromatography to afford product as a colorless oil (51%). 1H NMR (400 MHz, CDCl₃) δ 4.67 (s, 1H), 3.96 (s, 4H), 2.82 (t, J=6.4 Hz, 4H), 1.70 (t, J=6.4 Hz, 4H).

### Intermediate 4b: 7,10-dioxadispiro[2.2.46.23]dodecane

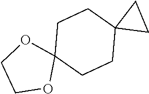 [SMILES: C1COC2(CCC3(CC3)CC2)O1]

To a solution of Intermediate 4a (5 g, 1.0 equiv.) in toluene (3 M) was added ZnEt2 (2.57 equiv.) dropwise at −40° C. and the mixture was stirred at −40° C. for 1 h. Then diiodomethane (6.0 equiv.) was added dropwise to the mixture at −40° C. under N2. The mixture was then stirred at 20° C. for 17 h under N2 atmosphere. The reaction mixture was poured into aq. NH4Cl at 0° C. and extracted 2× with EtOAc. The combined organic phases were washed with brine (20 mL), dried with anhydrous Na2SO4, filtered, and the filtrate was concentrated in vacuum. The residue was purified by column chromatography to afford product as a pale-yellow oil (73%).

### Intermediate 4c: spiro[2.5]octan-6-one

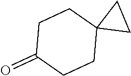

To a solution of Intermediate 4b (4 g, 1.0 equiv.) in 1:1 THF/H₂O (1.0 M) was added TFA (3.0 equiv.). The mixture was stirred at 20° C. for 2 h under N2 atmosphere. The reaction mixture was concentrated under reduced pressure to remove THF, and the residue adjusted pH to 7 with 2 M NaOH (aq.). The mixture was poured into water and 3× extracted with EtOAc. The combined organic phase was washed with brine, dried with anhydrous Na2SO4, filtered, and the filtrate was concentrated in vacuum. The residue was purified by column chromatography to afford product as a pale-yellow oil (68%). 1H NMR (400 MHz, CDCl3) δ 2.35 (t, J=6.6 Hz, 4H), 1.62 (t, J=6.6 Hz, 4H), 0.42 (s, 4H).

### Intermediate 4d: N-(4-methoxybenzyl)spiro[2.5]octan-6-amine

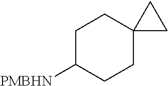

To a mixture of Intermediate 4c (2 g, 1.0 equiv.) and (4-methoxyphenyl)methanamine (1.1 equiv.) in DCM (0.3 M) was added AcOH (1.3 equiv.). The mixture was stirred at 20° C. for 1 h under N2 atmosphere. Then, NaBH(OAc)3 (3.3 equiv.) was added to the mixture at 0° C., and the mixture was stirred at 20° C. for 17 h under N2 atmosphere. The reaction mixture was concentrated under reduced pressure to remove DCM, and the resulting residue was diluted with H2O and extracted 3× with EtOAc. The combined organic layers were washed with brine, dried over Na₂SO4, filtered, and the filtrate was concentrated under reduced pressure to give a residue. The residue was purified by column chromatography to afford product as a gray solid (51%). 1H NMR (400 MHz, (CD3)2SO) δ 7.15-7.07 (m, 2H), 6.77-6.68 (m, 2H), 3.58 (s, 3H), 3.54 (s, 2H), 2.30 (ddt, J=10.1, 7.3, 3.7 Hz, 1H), 1.69-1.62 (m, 2H), 1.37 (td, J=12.6, 3.5 Hz, 2H), 1.12-1.02 (m, 2H), 0.87-0.78 (m, 2H), 0.13-0.04 (m, 2H).

### Intermediate 4e: spiro[2.5]octan-6-amine

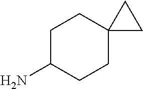

To a suspension of Pd/C (10% w/w, 1.0 equiv.) in MeOH (0.25 M) was added Intermediate 4d (2 g, 1.0 equiv.) and the mixture was stirred at 80° C. at 50 Psi for 24 h under H2 atmosphere. The reaction mixture was filtered, and the filtrate was concentrated under reduced pressure to give a residue that was purified by column chromatography to afford product as a white solid. 1H NMR (400 MHz, (CD3)2SO) δ 2.61 (tt, J=10.8, 3.9 Hz, 1H), 1.63 (ddd, J=9.6, 5.1, 2.2 Hz, 2H), 1.47 (td, J=12.8, 3.5 Hz, 2H), 1.21-1.06 (m, 2H), 0.82-0.72 (m, 2H), 0.14-0.05 (m, 2H).

### Intermediate 4f: ethyl 2-chloro-4-(spiro[2.5]octan-6-ylamino)pyrimidine-5-carboxylate

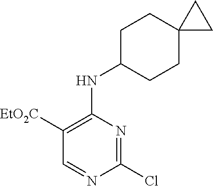 [SMILES: CCOC(=O)c1cnc(Cl)nc1NC1CCC2(CC1)CC2]

Intermediate 4f was synthesized (54%) from Intermediate 4e using the method employed in Intermediate 1e. 1H NMR (400 MHz, (CD3)2SO) δ 8.64 (s, 1H), 8.41 (d, J=7.9 Hz, 1H), 4.33 (q, J=7.1 Hz, 2H), 4.08 (d, J=9.8 Hz, 1H), 1.90 (dd, J=12.7, 4.8 Hz, 2H), 1.64 (t, J=12.3 Hz, 2H), 1.52 (q, J=10.7, 9.1 Hz, 2H), 1.33 (t, J=7.1 Hz, 3H), 1.12 (d, J=13.0 Hz, 2H), 0.40-0.21 (m, 4H).

### Intermediate 4g: 2-chloro-4-(spiro[2.5]octan-6-ylamino)pyrimidine-5-carboxylic acid

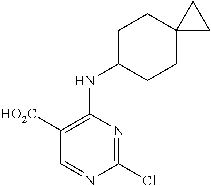 [SMILES: O=C(O)c1cnc(Cl)nc1NC1CCC2(CC1)CC2]

Intermediate 4g was synthesized (82%) from Intermediate 4f using the method employed in Intermediate 1f. 1H NMR (400 MHz, (CD3)2SO) δ 13.54 (s, 1H), 8.38 (d, J=8.0 Hz, 1H), 8.35 (s, 1H), 3.82 (qt, J=8.2, 3.7 Hz, 1H), 1.66 (dq, J=12.8, 4.1 Hz, 2H), 1.47-1.34 (m, 2H), 1.33-1.20 (m, 2H), 0.86 (dt, J=13.6, 4.2 Hz, 2H), 0.08 (dd, J=8.3, 4.8 Hz, 4H).

### Intermediate 4h: 2-chloro-9-(spiro[2.5]octan-6-yl)-7,9-dihydro-8H-purin-8-one

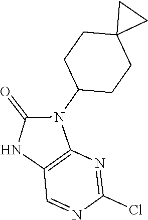 [SMILES: O=c1[nH]c2cnc(Cl)nc2n1C1CCC2(CC1)CC2]

Intermediate 4h was synthesized (67%) from Intermediate 4g using the method employed in Intermediate 1g. 1H NMR (400 MHz, (CD3)2SO) δ 11.68 (s, 1H), 8.18 (s, 1H), 4.26 (ddt, J=12.3, 7.5, 3.7 Hz, 1H), 2.42 (qd, J=12.6, 3.7 Hz, 2H), 1.95 (td, J=13.3, 3.5 Hz, 2H), 1.82-1.69 (m, 2H), 1.08-0.95 (m, 2H), 0.39 (tdq, J=11.6, 8.7, 4.2, 3.5 Hz, 4H).

### Intermediate 4i: 2-chloro-7-methyl-9-(spiro[2.5]octan-6-yl)-7,9-dihydro-8H-purin-8-one

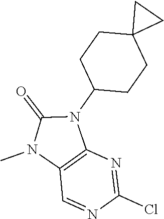 [SMILES: Cn1c(=O)n(C2CCC3(CC2)CC3)c2nc(Cl)ncc21]

Intermediate 4i was synthesized (67%) from Intermediate 4h using the method employed in Intermediate 1h. 1H NMR (400 MHz, CDCl₃) δ 7.57 (s, 1H), 4.03 (tt, J=12.5, 3.9 Hz, 1H), 3.03 (s, 3H), 2.17 (qd, J=12.6, 3.8 Hz, 2H), 1.60 (td, J=13.4, 3.6 Hz, 2H), 1.47-1.34 (m, 2H), 1.07 (s, 1H), 0.63 (dp, J=14.0, 2.5 Hz, 2H), −0.05 (s, 4H).

### Compound 4: 7-methyl-2-((7-methyl-[1,2,4]triazolo[1,5-a]pyridin-6-yl)amino)-9-(spiro[2.5]octan-6-yl)-7,9-dihydro-8H-purin-8-one (Compound 4)

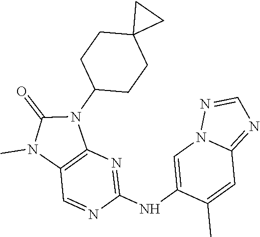 [SMILES: Cc1cc2ncnn2cc1Nc1ncc2c(n1)n(C1CCC3(CC1)CC3)c(=O)n2C]

Compound 4 was synthesized from Intermediate 4i and Intermediate 1d using the method employed in Compound 1. 1H NMR (400 MHz, (CD3)2SO) δ 9.09 (s, 1H), 8.73 (s, 1H), 8.44 (s, 1H), 8.16 (s, 1H), 7.78 (s, 1H), 4.21 (t, J=12.5 Hz, 1H), 3.36 (s, 3H), 2.43 (s, 3H), 2.34 (dt, J=13.0, 6.5 Hz, 2H), 1.93-1.77 (m, 2H), 1.77-1.62 (m, 2H), 0.91 (d, J=13.2 Hz, 2H), 0.31 (t, J=7.1 Hz, 2H). MS: 405.5 m/z [M+H].

### VII. FURTHER EXEMPLARY EMBODIMENTS

While the invention is described in conjunction with the illustrated embodiments, it is understood that they are not intended to limit the invention to those embodiments. On the contrary, the invention is intended to cover all alternatives, modifications, and equivalents, including equivalents of specific features, which may be included within the invention as defined by the appended claims.

Both the foregoing general description and detailed description, as well as the following examples, are exemplary and explanatory only and are not restrictive of the teachings. The section headings used herein are for organizational purposes only and are not to be construed as limiting the desired subject matter in any way. In the event that any literature incorporated by reference contradicts any term defined in this specification, this specification controls. All ranges given in the application encompass the endpoints unless stated otherwise.

The following non-limiting embodiments are also encompassed:
Embodiment 1. A method of producing multiple genome edits in an in vitro-cultured cell, comprising the steps of:
   a. contacting the cell in vitro with at least first and second lipid nucleic acid assembly compositions, wherein the first lipid nucleic acid assembly composition comprises a first guide RNA (gRNA) directed to a first target sequence and optionally a first nucleic acid genome editing tool and the second lipid nucleic acid assembly composition comprises a second gRNA directed to a second target sequence different from the first target sequence and optionally a nucleic acid genome editing tool;
   b. expanding the cell in vitro;
      thereby producing multiple genome edits in the cell.
Embodiment 2. The method of embodiment 1, wherein the cell is further contacted with at least one lipid nucleic acid assembly composition comprising a genome editing tool.
Embodiment 3. The method of embodiment 2, wherein the genome editing tool comprises a nucleic acid encoding an RNA-guided DNA binding agent.
Embodiment 4. The method of embodiment 1, wherein the cell is further contacted with a donor nucleic acid for insertion in a target sequence.
Embodiment 5. The method of any one of embodiments 1-4, wherein the lipid nucleic acid assembly compositions are administered sequentially.
Embodiment 6. The method any one of embodiment 1-4, wherein the lipid nucleic acid assembly compositions are administered simultaneously.
Embodiment 7. A method of delivering lipid nucleic acid assembly compositions to an in vitro-cultured cell, comprising the steps of:
   a. contacting the cell in vitro with at least a first lipid nucleic acid assembly composition comprising a first nucleic acid, thereby producing a contacted cell;
   b. culturing the contacted cell in vitro, thereby producing a cultured contacted cell;
   c. contacting the cultured contacted cell in vitro with at least a second lipid nucleic acid assembly composition comprising a second nucleic acid, wherein the second nucleic acid is different from the first nucleic acid; and
   d. expanding the cell in vitro;
      wherein the expanded cell exhibits increased survival.
Embodiment 8. The method of any one of embodiments 1-7, wherein the in vitro-cultured cell is a non-activated cell.
Embodiment 9. The method of any one of embodiments 1-7, wherein the in vitro-cultured cell is an activated cell.
Embodiment 10. The method of any one of embodiments 1-9, wherein the cell of (a) is activated after contact with at least one lipid nucleic acid assembly composition.
Embodiment 11. A method of producing multiple genome edits in an in vitro-cultured T cell, comprising the steps of:
   a. contacting the T cell in vitro with (i) a first lipid nucleic acid assembly composition comprising a guide RNA (gRNA) directed to a first target sequence and optionally (ii) one or two additional lipid nucleic acid assembly compositions, wherein each additional lipid nucleic acid assembly composition comprises a gRNA directed to a target sequence that differs from the first target sequence and/or a genome editing tool;
   b. activating the T cell in vitro;
   c. contacting the activated T cell in vitro with (i) a further nucleic acid assembly composition comprising a further guide RNA directed to a target sequence that differs from the target sequence(s) of (a) and optionally (ii) one or more further lipid nucleic acid assembly compositions, wherein each further lipid nucleic acid assembly composition comprises guide RNA directed to a target sequence that differs from the first and further target sequences and/or a genome editing tool;
   d. expanding the cell in vitro;
      thereby producing multiple genome edits in the cell.
Embodiment 12. The method of any one of the preceding embodiments, wherein the method comprises contacting the cell or T cell with at least 1, 2, 3, 4, 5, 6, 7, 8, 9, 10, or 11 lipid nucleic acid assembly compositions.
Embodiment 13. The method of any one of embodiments 11-12, wherein the cell or T cell of step (a) is contacted with two lipid nucleic acid assembly compositions, wherein the lipid nucleic acid assembly compositions are administered sequentially or simultaneously.
Embodiment 14. The method of any one of embodiments 11-12, wherein the cell or T cell of step (a) is contacted with three lipid nucleic acid assembly compositions, wherein the lipid nucleic acid assembly compositions are administered: (i) sequentially; (ii) simultaneously; or (iii) simultaneously (two compositions) and sequentially (one composition administered before or after).
Embodiment 15. The method of any one of embodiments 11-14, wherein the cell or T cell of step (c) is contacted with one to 8 lipid nucleic acid assembly compositions, optionally 1 to 4 lipid nucleic acid assembly compositions, wherein the lipid nucleic acid assembly compositions are administered: (i) sequentially; (ii) simultaneously; or (iii) simultaneously (at least two compositions) and sequentially (at least one composition administered before or after).
Embodiment 16. A method of genetically modifying a primary immune cell, comprising
   a. culturing a primary immune cell in a cell culture medium;
   b. providing a lipid nucleic acid assembly composition comprising a nucleic acid;
   c. combining in vitro the immune cell of (a) with the lipid nucleic acid assembly composition of (b);
   d. optionally, confirming the immune cell has been genetically modified; and
   e. optionally, proliferating the immune cell.
Embodiment 17. The method of embodiment 16, comprising carrying out the combining step (c) on a non-activated immune cell.
Embodiment 18. The method of embodiment 16 or 17, comprising carrying out the combining step (c) on an activated immune cell.
Embodiment 19. The method of embodiment 16, further comprising activating the immune cell after step (c).
Embodiment 20. The method of embodiment 16, further comprising
   (b2) providing a second lipid nucleic acid assembly composition comprising a second nucleic acid;
   (c2) combining in vitro the genetically modified immune cell of step (c) with the second lipid nucleic acid assembly composition;
   (d2) optionally, confirming the immune cell has been genetically modified using the second nucleic acid for genetic modification; and optionally, proliferating the immune cell.
Embodiment 21. The method of embodiment 20, further comprising
   (b3) providing a third lipid nucleic acid assembly composition comprising a third nucleic acid;
   (c3) combining in vitro the genetically modified immune cell of step (c2) with the third lipid nucleic acid assembly composition;
   (d2) optionally, confirming the immune cell has been genetically modified using the third nucleic acid for genetic modification; and
   (e) optionally, proliferating the immune cell.
Embodiment 22. The method of any one of embodiments 20-21, wherein steps (c) and (c2), and when present step (c3), are carried out sequentially.
Embodiment 23. The method of any one of embodiments 20-21, wherein steps (c) and (c2), and when present step (c3), are carried out simultaneously.
Embodiment 24. The method of any one of the preceding embodiments, wherein the nucleic acid or nucleic acid genome editing tool or gRNA comprises an RNA.
Embodiment 25. The method of any one of the preceding embodiments, wherein the nucleic acid or nucleic acid genome editing tool comprises a guide RNA (gRNA).
Embodiment 26. The method of any one of the preceding embodiments, wherein the nucleic acid or nucleic acid genome editing tool or gRNA comprises an sgRNA.
Embodiment 27. The method of any one of the preceding embodiments, wherein the nucleic acid or nucleic acid genome editing tool or gRNA comprises a dgRNA.
Embodiment 28. The method of any one of the preceding embodiments, wherein the nucleic acid or nucleic acid genome editing tool comprises an mRNA encoding a genome editing tool.
Embodiment 29. The method of any one of the preceding embodiments, wherein the nucleic acid or nucleic acid genome editing tool comprises a donor nucleic acid.
Embodiment 30. The method of any one of the preceding embodiments, wherein the nucleic acid or nucleic acid genome editing tool comprises an RNA-guided DNA binding agent.
Embodiment 31. The method of any one of the preceding embodiments, wherein the nucleic acid or nucleic acid genome editing tool comprises an RNA-guided DNA binding agent, and wherein the RNA-guided DNA binding agent is a Cas nuclease.
Embodiment 32. The method of any one of the preceding embodiments, wherein the nucleic acid or nucleic acid genome editing tool comprises an RNA-guided DNA binding agent, and wherein the RNA-guided DNA binding agent is Cas9.
Embodiment 33. The method of any one of the preceding embodiments, wherein the nucleic acid or nucleic acid genome editing tool comprises an RNA-guided DNA binding agent, and wherein the RNA-guided DNA binding agent is S. pyogenes Cas9.
Embodiment 34. The method of any one of the preceding embodiments, wherein the nucleic acid or nucleic acid genome editing tool comprises an RNA-guided DNA binding agent, and wherein the RNA-guided DNA binding agent is Cpf1.
Embodiment 35. The method of any one of the preceding embodiments, wherein the cell is a human cell.
Embodiment 36. The method of any one of the preceding embodiments, wherein the cell is a human peripheral blood mononuclear cell (PBMC).
Embodiment 37. The method of any one of the preceding embodiments, wherein the cell is a lymphocyte.
Embodiment 38. The method of any one of the preceding embodiments, wherein the cell is a T cell.
Embodiment 39. The method of any one of the preceding embodiments, wherein the cell is a CD4+ T cell.
Embodiment 40. The method of any one of the preceding embodiments, wherein the cell is a CD8+ T cell.
Embodiment 41. The method of any one of the preceding embodiments, wherein the cell is a memory T cell, or a naïve T cell.
Embodiment 42. The method of any one of the preceding embodiments, wherein the cell is a Tscm cell.
Embodiment 43. The method of any one of the preceding embodiments, wherein the cell is a B cell.
Embodiment 44. The method of any one of the preceding embodiments, wherein the cell is a memory B cell, or a naïve B cell.
Embodiment 45. The method of any one of the preceding embodiments, wherein the cell is a primary cell.
Embodiment 46. The method of any one of the preceding embodiments, wherein the lipid nucleic acid assembly composition is pretreated with a serum factor before contacting the cell, optionally wherein the serum factor is a primate serum factor, optionally a human serum factor.
Embodiment 47. The method of any one of the preceding embodiments, wherein the lipid nucleic acid assembly composition is pretreated with a human serum before contacting the cell.
Embodiment 48. The method of any one of the preceding embodiments, wherein the lipid nucleic acid assembly composition is pretreated with an ApoE before contacting the cell, optionally wherein the ApoE is a human ApoE.
Embodiment 49. The method of any one of the preceding embodiments, wherein the lipid nucleic acid assembly composition is pretreated with a recombinant ApoE3 or ApoE4 before contacting the cell, optionally wherein the ApoE3 or ApoE4 is a human ApoE3 or ApoE4.
Embodiment 50. The method of any one of the preceding embodiments, wherein the cell is serum-starved prior to contact with the lipid nucleic acid assembly composition or with the first lipid nucleic acid assembly composition.
Embodiment 51. The method of any one of the preceding embodiments, wherein the cell is cultured in a cell culture medium comprising one or more proliferative cytokines.
Embodiment 52. The method of any one of the preceding embodiments, wherein the cell is cultured in a cell culture medium comprising IL-2.
Embodiment 53. The method of any one of the preceding embodiments, wherein the cell is cultured in a cell culture medium comprising IL-7.
Embodiment 54. The method of any one of the preceding embodiments, wherein the cell is cultured in a cell culture medium comprising one or more or all of IL-2, IL-7, IL-15 and IL-21, and optionally one or more of an agent that provides activation through CD3 and/or CD28.
Embodiment 55. The method of any one of the preceding embodiments, wherein the cell is activated by exposing the cell to an antigen.
Embodiment 56. The method of any one of the preceding embodiments, wherein the cell is activated by polyclonal stimulation.
Embodiment 57. The method of any one of the preceding embodiments, wherein the method is carried out ex vivo.
Embodiment 58. The method of any one of the preceding embodiments, wherein the method further comprises contacting the cell with one or more donor nucleic acids.
Embodiment 59. The method of any one of the preceding embodiments, wherein the method further comprises contacting the cell with one or more donor nucleic acids, wherein the one or more donor nucleic acids comprise a vector.
Embodiment 60. The method of any one of the preceding embodiments, wherein the method further comprises contacting the cell with one or more donor nucleic acids, wherein the one or more donor nucleic acids comprise a viral vector.
Embodiment 61. The method of any one of the preceding embodiments, wherein the method further comprises contacting the cell with one or more donor nucleic acids, wherein the one or more donor nucleic acids comprise a lentiviral vector.
Embodiment 62. The method of any one of the preceding embodiments, wherein the method further comprises contacting the cell with one or more donor nucleic acids, wherein the one or more donor nucleic acids comprise an AAV.
Embodiment 63. The method of any one of the preceding embodiments, wherein the method further comprises contacting the cell with one or more donor nucleic acids, wherein at least one of the one or more donor nucleic acids is provided in a lipid nucleic acid assembly composition.
Embodiment 64. The method of any one of the preceding embodiments, wherein the method further comprises contacting the cell with one or more donor nucleic acids, wherein at least one of the one or more donor nucleic acids is integrated by homologous recombination.
Embodiment 65. The method of any one of the preceding embodiments, wherein the method further comprises contacting the cell with one or more donor nucleic acids, wherein at least one of the one or more donor nucleic acids comprise flanking nucleic acid regions homologous to all or part of the target sequence.
Embodiment 66. The method of any one of the preceding embodiments, wherein the method further comprises contacting the cell with one or more donor nucleic acids, wherein at least one of the one or more donor nucleic acids is integrated by blunt end insertion.
Embodiment 67. The method of any one of the preceding embodiments, wherein the method further comprises contacting the cell with one or more donor nucleic acids, wherein at least one of the one or more donor nucleic acids is integrated by non-homologous end joining.
Embodiment 68. The method of any one of the preceding embodiments, wherein the method further comprises contacting the cell with one or more donor nucleic acids, wherein the one or more donor nucleic acids is inserted into a safe harbor locus.
Embodiment 69. The method of any one of the preceding embodiments, wherein the method further comprises contacting the cell with one or more donor nucleic acids, wherein at least one of the one or more donor nucleic acids comprises regions having homology with corresponding regions of a T cell receptor sequence.
Embodiment 70. The method of any one of the preceding embodiments, wherein the method further comprises contacting the cell with one or more donor nucleic acids, wherein at least one of the one or more donor nucleic acids comprises regions having homology with corresponding regions of a TRAC locus, a B2M locus, an AAVS1 locus, and/or CIITA locus.
Embodiment 71. The method of any one of the preceding embodiments, wherein one of the lipid nucleic acid assembly compositions comprises a gRNA targeting TRAC.
Embodiment 72. The method of any one of the preceding embodiments, wherein one of the lipid nucleic acid assembly compositions comprises a gRNA targeting TRBC.
Embodiment 73. The method of any one of the preceding embodiments, wherein one of the lipid nucleic acid assembly compositions comprises a gRNA targeting B2M.
Embodiment 74. The method of any one of the preceding embodiments, wherein one of the lipid nucleic acid assembly compositions comprises a gRNA targeting TRAC, and one of the lipid nucleic acid assembly compositions comprises a gRNA targeting TRBC.
Embodiment 75. The method of any one of the preceding embodiments, wherein one of the lipid nucleic acid assembly compositions comprises a gRNA targeting TRAC, one of the lipid nucleic acid assembly compositions comprises a gRNA targeting TRBC, and one of the lipid nucleic acid assembly compositions comprises a gRNA targeting B2M.
Embodiment 76. The method of any one of the preceding embodiments, wherein the cell is a T cell, and wherein the method comprises reducing expression of an endogenous T cell receptor.
Embodiment 77. The method of any one of the preceding embodiments, wherein the cell is a T cell, and wherein the method comprises genetically modifying the T cell so as to express a genetically modified T cell receptor (TCR).
Embodiment 78. The method of any one of the preceding embodiments, wherein the method comprises contacting the cell with a donor nucleic acid, wherein the donor nucleic acid encodes a T cell receptor (TCR).
Embodiment 79. The method of any one of the preceding embodiments, wherein the method comprises contacting the cell with a donor nucleic acid, wherein the donor nucleic acid encodes the TCR WT1.
Embodiment 80. The method of any one of the preceding embodiments, wherein one of the lipid nucleic acid assembly compositions comprises a gRNA targeting TRAC, and one of the lipid nucleic acid assembly compositions comprises a gRNA targeting TRBC; wherein the method further comprises contacting the cell with a donor nucleic acid, wherein the donor nucleic acid encodes a TCR.
Embodiment 81. The method of the immediately preceding embodiment, wherein the TCR is the TCR WT1.
Embodiment 82. The method of any one of the preceding embodiments, wherein the lipid nucleic acid assembly composition is a lipid nanoparticle (LNP).
Embodiment 83. The method of any one of the preceding embodiments, wherein the lipid nucleic acid assembly composition is a lipoplex.
Embodiment 84. The method of any one of the preceding embodiments, wherein the lipid nucleic acid assembly composition comprises an ionizable lipid.
Embodiment 85. The method of any one of the preceding embodiments, wherein the ionizable lipid comprises a biodegradable ionizable lipid.
Embodiment 86. The method of any one of the preceding embodiments, wherein the ionizable lipid has a PK value in the range of pKa in the range of from about 5.1 to about 7.4, such as from about 5.5 to about 6.6, from about 5.6 to about 6.4, from about 5.8 to about 6.2, or from about 5.8 to about 6.5.
Embodiment 87. The method of any one of the preceding embodiments, wherein the lipid nucleic acid assembly composition comprises an amine lipid.
Embodiment 88. The method of any one of the preceding embodiments, wherein the lipid nucleic acid assembly composition comprises an amine lipid, wherein the amine lipid is Lipid A or its acetal analog.
Embodiment 89. The method of any one of the preceding embodiments, wherein the lipid nucleic acid assembly composition comprises a helper lipid.
Embodiment 90. The method of any one of the preceding embodiments, wherein the lipid nucleic acid assembly composition comprises a stealth lipid, optionally wherein:
   (i) the lipid nucleic acid assembly composition comprises a lipid component and the lipid component comprises: about 50-60 mol % amine lipid such as Lipid A, about 8-10 mol % neutral lipid; and about 2.5-4 mol % stealth lipid (e.g., a PEG lipid), wherein the remainder of the lipid component is helper lipid, and wherein the N/P ratio of the lipid nucleic acid assembly composition is about 6;
   (ii) the lipid nucleic acid assembly composition comprises about 50-60 mol % amine lipid such as Lipid A; about 27-39.5 mol % helper lipid; about 8-10 mol % neutral lipid; and about 2.5-4 mol % stealth lipid (e.g., a PEG lipid), wherein the N/P ratio of the lipid nucleic acid assembly composition is about 5-7 (e.g., about 6);
   (iii) the lipid nucleic acid assembly composition comprises a lipid component and the lipid component comprises: about 50-60 mol % amine lipid such as Lipid A; about 5-15 mol % neutral lipid; and about 2.5-4 mol % Stealth lipid (e.g., a PEG lipid), wherein the remainder of the lipid component is helper lipid, and wherein the N/P ratio of the lipid nucleic acid assembly composition is about 3-10;
   (iv) the lipid nucleic acid assembly composition comprises a lipid component and the lipid component comprises: about 40-60 mol % amine lipid such as Lipid A; about 5-15 mol % neutral lipid; and about 2.5-4 mol % Stealth lipid (e.g., a PEG lipid), wherein the remainder of the lipid component is helper lipid, and wherein the N/P ratio of the lipid nucleic acid assembly composition is about 6;
   (v) the lipid nucleic acid assembly composition comprises a lipid component and the lipid component comprises: about 50-60 mol % amine lipid such as Lipid A; about 5-15 mol % neutral lipid; and about 1.5-10 mol % Stealth lipid (e.g., a PEG lipid), wherein the remainder of the lipid component is helper lipid, and wherein the N/P ratio of the lipid nucleic acid assembly composition is about 6;
   (vi) the lipid nucleic acid assembly composition comprises a lipid component and the lipid component comprises: about 40-60 mol % amine lipid such as Lipid A; about 0-10 mol % neutral lipid; and about 1.5-10 mol % Stealth lipid (e.g., a PEG lipid), wherein the remainder of the lipid component is helper lipid, and wherein the N/P ratio of the lipid nucleic acid assembly composition is about 3-10;
   (vii) the lipid nucleic acid assembly composition comprises a lipid component and the lipid component comprises: about 40-60 mol % amine lipid such as Lipid A; less than about 1 mol % neutral lipid; and about 1.5-10 mol % Stealth lipid (e.g., a PEG lipid), wherein the remainder of the lipid component is helper lipid, and wherein the N/P ratio of the lipid nucleic acid assembly composition is about 3-10;
   (viii) the lipid nucleic acid assembly composition comprises a lipid component and the lipid component comprises: about 40-60 mol % amine lipid such as Lipid A; and about 1.5-10 mol % Stealth lipid (e.g., a PEG lipid), wherein the remainder of the lipid component is helper lipid, wherein the N/P ratio of the LNP composition is about 3-10, and wherein the lipid nucleic acid assembly composition is essentially free of or free of neutral phospholipid; or
   (ix) the lipid nucleic acid assembly composition comprises a lipid component and the lipid component comprises: about 50-60 mol % amine lipid such as Lipid A; about 8-10 mol % neutral lipid; and about 2.5-4 mol % Stealth lipid (e.g., a PEG lipid), wherein the remainder of the lipid component is helper lipid, and wherein the N/P ratio of the lipid nucleic acid assembly composition is about 3-7.
Embodiment 91. The method of any one of the preceding embodiments, wherein the lipid nucleic acid assembly composition comprises a neutral lipid.
Embodiment 92. The method of any one of the preceding embodiments, wherein the neutral lipid is present in the lipid nucleic acid assembly composition at about 9 mol %.
Embodiment 93. The method of any one of the preceding embodiments, wherein the amine lipid is present in the lipid nucleic acid assembly composition at about 50 mol %.
Embodiment 94. The method of any one of the preceding embodiments, wherein the stealth lipid is present in the lipid nucleic acid assembly composition at about 3 mol %.
Embodiment 95. The method of any one of the preceding embodiments, wherein the helper lipid is present in the lipid nucleic acid assembly composition at about 38 mol %.
Embodiment 96. The method of any one of the preceding embodiments wherein the N/P ratio of the lipid nucleic acid assembly composition is about 6.
Embodiment 97. The method of any one of the preceding embodiments, wherein the lipid nucleic acid assembly composition comprises an amine lipid, a helper lipid, and a PEG lipid.
Embodiment 98. The method of any one of the preceding embodiments, wherein the lipid nucleic acid assembly composition comprises an amine lipid, a helper lipid, a neutral lipid, and a PEG lipid.
Embodiment 99. The method of any one of the preceding embodiments, wherein the lipid nucleic acid assembly composition comprises a lipid component and the lipid component comprises: about 50 mol % amine lipid such as Lipid A; about 9 mol % neutral lipid such as DSPC; about 3 mol % of stealth lipid such as a PEG lipid, such as PEG2k-DMG, and the remainder of the lipid component is helper lipid such as cholesterol wherein the N/P ratio of the lipid nucleic acid assembly composition is about 6.
Embodiment 100. The method of any one of the preceding embodiments, wherein the amine lipid is Lipid A.
Embodiment 101. The method of any one of the preceding embodiments, wherein the neutral lipid is DSPC.
Embodiment 102. The method of any one of the preceding embodiments, wherein the stealth lipid is PEG2k-DMG.
Embodiment 103. The method of any one of the preceding embodiments, wherein the helper lipid is cholesterol.
Embodiment 104. The method of any one of the preceding embodiments, wherein the lipid nucleic acid assembly composition comprises a lipid component and the lipid component comprises: about 50 mol % Lipid A; about 9 mol % DSPC; about 3 mol % of PEG2k-DMG, and the remainder of the lipid component is cholesterol wherein the N/P ratio of the lipid nucleic acid assembly composition is about 6.
Embodiment 105. The method of any one of the preceding embodiments, wherein the LNP has a diameter of about 1-250 nm, 10-200 nm, about 20-150 nm, about 50-150 nm, about 50-100 nm, about 50-120 nm, about 60-100 nm, about 75-150 nm, about 75-120 nm, or about 75-100 nm.
Embodiment 106. The method of any one of the preceding embodiments, wherein the LNP composition comprises a population of the LNP with an average diameter of about 10-200 nm, about 20-150 nm, about 50-150 nm, about 50-100 nm, about 50-120 nm, about 60-100 nm, about 75-150 nm, about 75-120 nm, or about 75-100 nm.
Embodiment 107. The method of any one of the preceding embodiments, wherein the lipid nucleic acid assembly composition comprises:
   a. about 40-60 mol % amine lipid;
   b. about 5-15 mol % neutral lipid; and
   c. about 1.5-10 mol % PEG lipid,

wherein the remainder of the lipid component is helper lipid, and wherein the N/P ratio of the LNP composition is about 3-10.
Embodiment 108. The method of any one of the preceding embodiments, wherein the lipid nucleic acid assembly composition comprises:
   a. about 50-60 mol % amine lipid;
   b. about 8-10 mol % neutral lipid; and
   c. about 2.5-4 mol % PEG lipid,
      wherein the remainder of the lipid component is helper lipid, and wherein the N/P ratio of the LNP composition is about 3-8.
Embodiment 109. The method of any one of the preceding embodiments, wherein the lipid nucleic acid assembly composition comprises:
   a. about 50-60 mol % amine lipid;
   b. about 5-15 mol % DSPC; and
   c. about 2.5-4 mol % PEG lipid,
      wherein the remainder of the lipid component is cholesterol, and
      wherein the N/P ratio of the LNP composition is 3-8±0.2.
Embodiment 110. The method of any one of the preceding embodiments wherein the average diameter is a Z-average diameter.
Embodiment 111. The method of any one of the preceding embodiments, wherein the genetically modified cell:
   a. comprises a genetic modification to decrease expression of a gene;
   b. comprises a genetic modification comprising insertion of a donor nucleic acid;
   c. exhibits increased secretion of cytokines (IL-2, IFNγ, and/or TNFα);
   d. exhibits increased cytotoxicity;
   e. exhibits increased memory cell phenotype;
   f. exhibits increased expansion;
   g. exhibits longer duration of proliferation to repeated stimulation; and/or
   h. exhibits decreased translocation events.
Embodiment 112. The method of any one of the preceding embodiments, wherein the contacted cell exhibits increased survival, wherein increased survival is a post-transfection cell survival rate of at least 70%.
Embodiment 113. The method of any one of the preceding embodiments, wherein the contacted cell exhibits increased survival, wherein increased survival is a post-transfection cell survival rate of at least 80%.
Embodiment 114. The method of any one of the preceding embodiments, wherein the contacted cell exhibits increased survival, wherein increased survival is a post-transfection cell survival rate of at least 90%.
Embodiment 115. The method of any one of the preceding embodiments, wherein the contacted cell exhibits increased survival, wherein increased survival is a post-transfection cell survival rate of at least 95%.
Embodiment 116. The method of any one of the preceding embodiments, wherein the contacted cell has fewer than 1% translocations post-editing.
Embodiment 117. The method of any one of the preceding embodiments, wherein the percent editing efficiency rate is at least 60% for each gRNA target site.
Embodiment 118. The method of any one of the preceding embodiments, wherein the percent editing efficiency rate is at least 70% for each gRNA target site.
Embodiment 119. The method of any one of the preceding embodiments, wherein the percent editing efficiency rate is at least 80% for each gRNA target site.
Embodiment 120. The method of any one of the preceding embodiments, wherein the percent editing efficiency rate is at least 90% for each gRNA target site.
Embodiment 121. The method of any one of the preceding embodiments, wherein the percent editing efficiency rate is at least 95% for each gRNA target site.
Embodiment 122. The method of any one of the preceding embodiments, wherein the contacted cell is a T cell, and wherein the contacted T cell expresses CD27 and CD45RA by standard flow cytometry methods.
Embodiment 123. The method of any one of the preceding embodiments, further comprising proliferating the cell to form a population of cells that comprise the genetic modification.
Embodiment 124. The method of any one of the preceding embodiments, wherein the edit or modification is not transient.
Embodiment 125. The method of any one of the preceding embodiments, wherein the genetically modified cell is for use in therapy.
Embodiment 126. The method of any one of the preceding embodiments, wherein the genetically modified cell is for use in cancer therapy.
Embodiment 127. An immune cell which has been genetically modified, obtainable using the method of any one of embodiments 1 to 124.
Embodiment 128. A composition, comprising the cell of embodiment 127.
Embodiment 129. A method of therapy, comprising administering to a patient the cell according to claim 127 or a composition according to embodiment 128.
Embodiment 130. A method of therapy according to embodiment 129, for treatment of cancer.
Embodiment 131. The method of embodiment 130, wherein the cell expresses a TCR with specificity for a polypeptide expressed by cells of the cancer.
Embodiment 132. A method of therapy, comprising carrying out an ex vivo method according to any of embodiments 1-124.
Embodiment 133. A method of therapy, comprising carrying out a method according to any of embodiments 1-124.
Embodiment 134. A method of therapy according to embodiment 132 or 133, for treatment of cancer.
Embodiment 135. A method of creating a cell bank, comprising genetically modifying a cell, e.g., an immune cell using a method according to any of embodiments 1 to 126 to obtain a population of genetically modified cells, and transferring the genetically modified cells into a cell bank.
Embodiment 136. A method according to embodiment 135, comprising creating a first population of cells, e.g., immune cells, comprising a first genetic modification; dividing the first population into at least first and second sub-populations and carrying out further, different genetic modification of each according to any of claims preceding claims so that the first and second sub-populations have at least one common genetic modification and at least one different genetic modification.
Embodiment 137. A method according to embodiment 136, comprising transferring the first and second sub-populations into the cell bank.
Embodiment 138. A cell or population of cells produced by the method of any one of embodiments 1 to 124 for use as an adoptive cell transfer (ACT) therapy.
Embodiment 139. A cell or population of cells produced by the method of any one of embodiments 1 to 124 for use as an ACT therapy, wherein the cell or population of cells has increased post-editing survival rate.
Embodiment 140. A cell or population of cells produced by the method of any one of embodiments 1 to 124 for use as an ACT therapy, wherein the cell or population of cells has low toxicity.
Embodiment 141. A cell or population of cells produced by the method of any one of embodiments 1 to 124 for use as an ACT therapy, wherein the cell or population of cells has fewer than 2% translocations.
Embodiment 142. A cell or population of cells produced by the method of any one of embodiments 1 to 124 for use as an ACT therapy, wherein the cell or population of cells has no measurable target-target translocations.
Embodiment 143. A cell or population of cells produced by the method of any one of embodiments 1 to 124 for use as an ACT therapy, wherein the cell or population of cells has increased production of cytokines (IL-2, IFNγ, and/or TNFα).
Embodiment 144. A cell or population of cells produced by the method of any one of embodiments 1 to 124 for use as an ACT therapy, wherein the cell or population of cells has enhanced durability of response with repeated stimulations.
Embodiment 145. A cell or population of cells produced by the method of any one of embodiments 1 to 124 for use as an ACT therapy, wherein the cell or population of cells has increased expansion.
Embodiment 146. A cell or population of cells produced by the method of any one of embodiments 1 to 124 for use as an ACT therapy, wherein the cell or population of cells has memory cell phenotype.
Embodiment 147. A cell or population of cells produced by the method of any one of embodiments 1 to 124 for use as an ACT therapy, wherein the cell or population of cells has comparable insertion rates with alternative methods such as electroporation.
Embodiment 148. A cell or population of cells produced by the method of any one of embodiments 1 to 124 for use as an ACT therapy, wherein the cell or population of cells has reduced number or percentage of unedited cells.
Embodiment 149. A cell or population of cells produced by the method of any one of embodiments 1 to 124 for use as an ACT therapy, wherein the cell or population of cells has improved cytotoxicity.
Embodiment 150. A cell or population of cells produced by the method of any one of embodiments 1 to 124 for use as an ACT therapy, wherein the cell or population of cells has improved proliferation.
Embodiment 151. A pharmaceutical composition comprising the cell or cell population of any one of embodiments 138-150.
Embodiment 152. A method of adoptive cell therapy (ACT) in a subject in need thereof, comprising administering the cell or population of any one of embodiments 138-150

The following non-limiting embodiments are also encompassed:

Embodiment 01 A method of genetically modifying a primary immune cell, comprising
a. culturing a primary immune cell in a cell culture medium;
b. providing a lipid nucleic acid assembly composition comprising a nucleic acid;
c. combining in vitro the immune cell of (a) with the lipid nucleic acid assembly composition of (b);
d. optionally, confirming the immune cell has been genetically modified; and
e. optionally, proliferating the immune cell.
   Embodiment 02 A method according to embodiment 1, comprising carrying out the combining step (c) on a non-activated immune cell.
   Embodiment 03 A method according to embodiment 1, comprising carrying out the combining step (c) on an activated immune cell.
   Embodiment 04 A method according to any previous embodiment, further comprising activating the immune cell after step (c).
   Embodiment 05 A method according to embodiment 4, wherein the activating step comprises exposing the immune cell to antigen.
   Embodiment 06 A method according to any previous embodiment, wherein the culturing step comprises one or more proliferative cytokines, for example one or more or all of IL-2, IL-15 and IL-21, and/or one or more agents that provides activation through CD3 and/or CD28.
   Embodiment 07 A method according to any previous embodiment, further comprising proliferating the immune cell to form a population of immune cells that comprise the genetic modification.
   Embodiment 08 A method according to any previous embodiment, wherein the cell:
a. comprises a genetic modification to decrease expression of a gene;
b. comprises a genetic modification comprising insertion of a donor
nucleic acid construct;
c. exhibits increased secretion of cytokines (IL-2, interferon-gamma, TNF-α, etc.);
d. exhibits increased cytotoxicity;
e. exhibits increase memory cell phenotype;
f. exhibits increased expansion;
g. exhibits longer duration of proliferation to repeated stimulation; and/or
h. exhibits decreased translocation events.
   Embodiment 09 A method according to any previous embodiment, wherein the immune cell is a lymphocyte, such as a T cell or a B cell.
   Embodiment 10 A method according to any previous embodiment, further comprising
(b2) providing a second lipid nucleic acid assembly composition comprising a second nucleic acid;
(c2) combining in vitro the genetically modified immune cell of step (c) with the second lipid nucleic acid assembly composition;
(d2) optionally, confirming the immune cell has been genetically modified using the second nucleic acid for genetic modification; and optionally, proliferating the immune cell.
   Embodiment 11 A method according to embodiment 10, further comprising
(b3) providing a third lipid nucleic acid assembly composition comprising a third nucleic acid;
(c3) combining in vitro the genetically modified immune cell of step (c2) with the third lipid nucleic acid assembly composition;
(d2) optionally, confirming the immune cell has been genetically modified using the third nucleic acid for genetic modification; and
(e) optionally, proliferating the immune cell.
   Embodiment 12 A method according to any of embodiments 10 to 11, wherein steps (c) and (c2), and when present step (c3), are carried out sequentially.
   Embodiment 13 A method according to any of embodiments 10 to 11, wherein steps (c) and (c2), and when present step (c3), are carried out simultaneously.
   Embodiment 14 A method according to any previous embodiment, wherein the nucleic acid is a guide sequence for a genetic modification carried out by an RNA-guided DNA binding agent.
   Embodiment 15 A method according to embodiment 14, wherein the RNA-guided DNA binding agent is a CRISPR/Cas9 protein.
   Embodiment 16 A method according to any previous embodiment, wherein the lipid nucleic acid assembly composition further comprises a vector encoding a donor template.
   Embodiment 17 A method according to embodiment 16, wherein the donor template comprises regions having homology with corresponding regions of a T cell receptor locus.
   Embodiment 18 A method according to any of embodiments 16 to 17, wherein the donor template comprises regions having homology with corresponding regions of a TRAC locus, a B2M locus, an AAVS1 locus, and/or CIITA locus.
   Embodiment 19 A method according to any previous embodiment, wherein a plurality of genetic modifications are carried out on the immune cell prior to activation of the immune cell.
   Embodiment 20 A method according to any previous embodiment, wherein the immune cell is a human cell.
   Embodiment 21 A method according to any previous embodiment, wherein the immune cell is a memory T cell, or a naïve T cell.
   Embodiment 22 A method according to any previous embodiment, wherein the immune cell is a CD4+ T cell.
   Embodiment 23 A method according to any previous embodiment, wherein the immune cell is a CD8+ T cell.
   Embodiment 24 A method according to any previous embodiment, wherein the immune cell is a B cell.
   Embodiment 25 A method according to any previous embodiment, wherein the method is an ex vivo method.
   Embodiment 26 A method according to any previous embodiment, further comprising combining the lipid nucleic acid assembly composition with a serum factor.
   Embodiment 27 A method according to embodiment 26, wherein combining the lipid nucleic acid assembly composition with a serum factor occurs before combining the composition with the immune cell.
   Embodiment 28 A method according to embodiment 26 or 27, wherein the serum factor is ApoE.
   Embodiment 29 A method according to embodiment 28, wherein the serum factor is a recombinant ApoE3 or ApoE4.
   Embodiment 30 A method according any of embodiments 26 to 27, wherein the serum factor is comprised by primate serum, such as human serum.
   Embodiment 31 A method according to any previous embodiment, comprising genetically modifying a T cell so as to express a genetically modified T cell receptor.
   Embodiment 32 A method according to any previous embodiment, comprising reducing expression of an endogenous T cell receptor.
   Embodiment 33 A method according to any previous embodiment, wherein the genetically modified immune cell is for use in therapy.
   Embodiment 34 A method according to any previous embodiment, wherein the genetically modified immune cell is for use in cancer therapy.
   Embodiment 35 A method of creating a cell bank, comprising genetically modifying an immune cell using a method according to any previous embodiment to obtain a population of genetically modified cells, and transferring the genetically modified cells into a cell bank.
   Embodiment 36 A method according to embodiment 35, comprising creating a first population of immune cells comprising a first genetic modification; dividing the first population into at least first and second sub-populations and carrying out further, different genetic modification of each according to any of embodiments 1 to 34 so that the first and second sub-populations have at least one common genetic modification and at least one different genetic modification.
   Embodiment 37 A method according to embodiment 36, comprising transferring the first and second sub-populations into the cell bank.
   Embodiment 38 An immune cell which has been genetically modified, obtainable using the method of any of embodiments 1 to 34.
   Embodiment 39 An immune cell according to embodiment 38, which has been genetically modified to introduce at least 3 separate genetic modifications.
   Embodiment 40 A composition, comprising an immune cell according to embodiments 38 or 39.
   Embodiment 41 A method of therapy, comprising administering to a patient an immune cell according to any of embodiments 38 to 39 or a composition according to embodiment 40.
   Embodiment 42 A method of therapy according to embodiment 41, for treatment of cancer.
   Embodiment 43 A method of therapy, comprising carrying out an ex vivo method according to any of embodiments 1 to 34.
   Embodiment 44 A method of therapy, comprising carrying out a method according to any of embodiments 1 to 34.
   Embodiment 45 A method of therapy according to embodiment 43 or 44, for treatment of cancer.

The following non-limiting embodiments are also encompassed:
Embodiment_A 1. A method of producing multiple genome edits in an in vitro-cultured cell, comprising the steps of:
   a. contacting the cell in vitro with at least first and second lipid nucleic acid assembly compositions, wherein the first lipid nucleic acid assembly composition comprises a first guide RNA (gRNA) directed to a first target sequence and optionally a nucleic acid genome editing tool and the second lipid nucleic acid assembly composition comprises a second gRNA directed to a second target sequence different from the first target sequence and optionally a nucleic acid genome editing tool;
   b. expanding the cell in vitro;
      thereby producing multiple genome edits in the cell.
Embodiment_A 2. The method of embodiment_A 1, wherein the cell is contacted with at least one lipid nucleic acid assembly composition comprising a genome editing tool.
Embodiment_A 3. The method of embodiment_A 2, wherein the genome editing tool comprises a nucleic acid encoding an RNA-guided DNA binding agent.
Embodiment_A 4. The method of embodiment_A 1, wherein the cell is further contacted with a donor nucleic acid for insertion in a target sequence.
Embodiment_A 5. The method of any one of embodiments_A 1-4, wherein the lipid nucleic acid assembly compositions are administered sequentially.
Embodiment_A 6. The method any one of embodiments_A 1-4, wherein the lipid nucleic acid assembly compositions are administered simultaneously.
Embodiment_A 7. A method of delivering lipid nucleic acid assembly compositions to an in vitro-cultured cell, comprising the steps of:
   a. contacting the cell in vitro with at least a first lipid nucleic acid assembly composition comprising a first nucleic acid, thereby producing a contacted cell;
   b. culturing the contacted cell in vitro, thereby producing a cultured contacted cell;
   c. contacting the cultured contacted cell in vitro with at least a second lipid nucleic acid assembly composition comprising a second nucleic acid, wherein the second nucleic acid is different from the first nucleic acid; and
   d. expanding the cell in vitro;
      wherein the expanded cell exhibits increased survival.
Embodiment_A 8. The method of embodiment 7, wherein the expanded cell has a survival rate of at least 70%, optionally the survival rate is at least 70% at 24 hours of expansion.
Embodiment_A 9. The method of any one of embodiments_A 1-8, wherein the cell is contacted with 2-12 lipid nucleic acid assembly compositions.
Embodiment_A 10. The method of any one of embodiments_A 1-8, wherein the cell is contacted with 2-8 lipid nucleic acid assembly compositions.
Embodiment_A 11. The method of any one of embodiments_A 1-8, wherein the cell is contacted with 2-6 lipid nucleic acid assembly compositions.
Embodiment_A 12. The method of any one of embodiments_A 1-8, wherein the cell is contacted with 3-8 lipid nucleic acid assembly compositions.
Embodiment_A 13. The method of any one of embodiments_A 1-8, wherein the cell is contacted with 3-6 lipid nucleic acid assembly compositions.
Embodiment_A 14. The method of any one of embodiments_A 1-8, wherein the cell is contacted with 4-6 lipid nucleic acid assembly compositions.
Embodiment_A 15. The method of any one of embodiments_A 1-8, wherein the cell is contacted with 6-12 lipid nucleic acid assembly compositions.
Embodiment_A 16. The method of any one of embodiments_A 1-8, wherein the cell is contacted with 3, 4, 5, or 6 lipid nucleic acid assembly compositions.
Embodiment_A 17. The method of any one of embodiments_A 1-8, wherein the cell is contacted with the lipid nucleic acid assembly compositions simultaneously.
Embodiment_A 18. The method of any one of embodiments_A 1-8, wherein the cell is contacted with no more than 6 lipid nucleic acid assembly compositions simultaneously.
Embodiment_A 19. The method of any one of embodiments_A 1-8, wherein the cell is contacted with no more than 2 lipid nucleic acid assembly compositions simultaneously.
Embodiment_A 20. A method of gene editing in a cell, comprising the steps of:
   a. contacting the cell in vitro with a first lipid nucleic acid assembly composition comprising a first genome editing tool and a second lipid nucleic acid assembly composition comprising a second genome editing tool; and
   b. expanding the cell in vitro;
      thereby editing the cell.
Embodiment_A 21. The method of embodiment_A 20, wherein the first genome editing tool comprises a guide RNA.
Embodiment_A 22. The method of any one of embodiments_A 20-21, further comprising contacting the cell in vitro with a third lipid nucleic acid assembly composition comprise a genome editing tool, and wherein at least two lipid nucleic acid assembly compositions comprise a gRNA.
Embodiment_A 23. The method of any one of embodiments_A 20-22, wherein at least one lipid nucleic acid assembly composition comprises an RNA-guided DNA binding agent.
Embodiment_A 24. The method of embodiment_A 23, wherein the RNA-guided DNA binding agent is a Cas9.
Embodiment_A 25. The method of any one of embodiments_A 20-24, further comprising contacting the cell with a donor nucleic acid.
Embodiment_A 26. The method of any one of embodiments_A 20-25, wherein the second genome editing tool is an RNA-guided DNA binding agent, such as an S. pyogenes Cas9.
Embodiment_A 27. The method of any one of embodiment_A 1-26, wherein the cell is an immune cell.
Embodiment_A 28. The method of any one of embodiment_A 1-27, wherein the cell is a lymphocyte.
Embodiment_A 29. The method of any one of embodiments_A 1-28, wherein the cell is a T cell.
Embodiment_A 30. The method of any one of embodiments_A 1-29, wherein the cell is a non-activated cell.
Embodiment_A 31. The method of any one of embodiments_A 1-29, wherein the cell is an activated cell.
Embodiment_A 32. The method of any one of embodiments_A 1-31, wherein the cell of (a) is activated after contact with at least one lipid nucleic acid assembly composition.
Embodiment_A 33. A method of producing multiple genome edits in an in vitro-cultured T cell, comprising the steps of:
   a. contacting the T cell in vitro with (i) a first lipid nucleic acid assembly composition comprising a guide RNA (gRNA) directed to a first target sequence and optionally (ii) one or two additional lipid nucleic acid assembly compositions, wherein each additional lipid nucleic acid assembly composition comprises a gRNA directed to a target sequence that differs from the first target sequence and/or a genome editing tool;
   b. activating the T cell in vitro;
   c. contacting the activated T cell in vitro with (i) a further nucleic acid assembly composition comprising a further guide RNA directed to a target sequence that differs from the target sequence(s) of (a) and optionally (ii) one or more lipid nucleic acid assembly compositions, wherein each lipid nucleic acid assembly composition comprises a guide RNA directed to a target sequence that differs from the target sequence(s) of (a) and from each other and/or a genome editing tool;
   d. expanding the cell in vitro;
      thereby producing multiple genome edits in the T cell.
Embodiment_A 34. The method of any one of the preceding embodiments_A, wherein the method comprises contacting the cell or T cell with at least 1, 2, 3, 4, 5, 6, 7, 8, 9, 10, or 11 lipid nucleic acid assembly compositions
Embodiment_A 35. The method of any one of the preceding embodiments_A, wherein the method comprises contacting the cell or T cell with 4-12 or 4-8 lipid nucleic acid assembly compositions.
Embodiment_A 36. The method of any one of embodiments_A 33-35, wherein the cell or T cell of step (a) is contacted with two lipid nucleic acid assembly compositions, wherein the lipid nucleic acid assembly compositions are administered sequentially or simultaneously.
Embodiment_A 37. The method of any one of embodiments_A 33-36, wherein the cell or T cell of step (a) is contacted with three lipid nucleic acid assembly compositions, wherein the lipid nucleic acid assembly compositions are administered: (i) sequentially; (ii) simultaneously; or (iii) simultaneously (two compositions) and sequentially (one composition administered before or after).
Embodiment_A 38. The method of any one of embodiments_A 33-37, wherein the cell or T cell of step (c) is contacted with one to 8 lipid nucleic acid assembly compositions, optionally 1 to 4 lipid nucleic acid assembly compositions, wherein the lipid nucleic acid assembly compositions are administered: (i) sequentially; (ii) simultaneously; or (iii) simultaneously (at least two compositions) and sequentially (at least one composition administered before or after).
Embodiment_A 39. The method of any one of the preceding embodiments_A, wherein one of the lipid nucleic acid assembly compositions comprises a gRNA targeting a gene that reduces or eliminates surface expression of MHC class I.
Embodiment_A 40. The method of any one of the preceding embodiments_A, wherein one of the lipid nucleic acid assembly compositions comprises a gRNA targeting B2M.
Embodiment_A 41. The method of any one of the preceding embodiments_A, wherein one of the lipid nucleic acid assembly compositions comprises a gRNA targeting a gene that reduces or eliminates surface expression of HLA-A.
Embodiment_A 42. The method of any one of the preceding embodiments_A, wherein one of the lipid nucleic acid assembly compositions comprises a gRNA targeting HLA-A.
Embodiment_A 43. The method of embodiment 98, wherein the cell is homozygous for HLA-B and homozygous for HLA-C.
Embodiment_A 44. The method of any one of the preceding embodiments_A, wherein one of the lipid nucleic acid assembly compositions comprises a gRNA targeting a gene that reduces or eliminates surface expression of MHC class II.
Embodiment_A 45. The method of any one of the preceding embodiments_A, wherein one of the lipid nucleic acid assembly compositions comprises a gRNA targeting CIITA.
Embodiment_A 46. The method of any one of the preceding embodiments_A, wherein one of the lipid nucleic acid assembly compositions comprises a gRNA targeting TRAC, and one of the lipid nucleic acid assembly compositions comprises a gRNA targeting TRBC.
Embodiment_A 47. The method of any one of the preceding embodiments_A, wherein one of the lipid nucleic acid assembly compositions comprises a gRNA targeting TRAC, one of the lipid nucleic acid assembly compositions comprises a gRNA targeting TRBC, and a further lipid nucleic acid assembly composition comprises a gRNA targeting B2M.
Embodiment_A 48. The method of any one of the preceding embodiments_A, wherein one of the lipid nucleic acid assembly compositions comprises a gRNA targeting TRAC, one of the lipid nucleic acid assembly compositions comprises a gRNA targeting TRBC, and a further lipid nucleic acid assembly composition comprises a gRNA targeting HLA-A.
Embodiment_A 49. The method of any one of the preceding embodiments_A, wherein one of the lipid nucleic acid assembly compositions comprises a gRNA targeting TRAC, one of the lipid nucleic acid assembly compositions comprises a gRNA targeting TRBC, a further lipid nucleic acid assembly composition comprises a gRNA targeting B2M, and a further lipid nucleic acid assembly composition comprises a gRNA targeting CIITA.
Embodiment_A 50. The method of any one of the preceding embodiments_A, wherein one of the lipid nucleic acid assembly compositions comprises a gRNA targeting TRAC, one of the lipid nucleic acid assembly compositions comprises a gRNA targeting TRBC, a further lipid nucleic acid assembly composition comprises a gRNA targeting HLA-A, and a further lipid nucleic acid assembly composition comprises a gRNA targeting CIITA.
Embodiment_A 51. The method of any one of embodiments_A 94-106, wherein a further lipid nucleic acid assembly composition comprises an RNA guided DNA binding agent, optionally Cas9.
Embodiment_A 52. The method of any one of embodiments_A 94-107, wherein a further lipid nucleic acid assembly composition comprises a donor nucleic acid.
Embodiment_A 53. The method of embodiment 108, wherein the donor nucleic acid comprises a targeting receptor.
Embodiment_A 54. The method of any one of the preceding embodiments_A, wherein the lipid nucleic acid assembly composition comprises an amine lipid, wherein the amine lipid is Lipid A or its acetal analog; an amine lipid provided in WO2020219876, or wherein the amine lipid is Lipid D or an amine lipid provided in WO2020072605
Embodiment_A 55. The method of any one of the preceding embodiments_A, wherein the lipid nucleic acid assembly composition comprises a helper lipid.
Embodiment_A 56. The method of any one of the preceding embodiments_A, wherein the lipid nucleic acid assembly composition comprises a stealth lipid, optionally wherein:
   (i) the lipid nucleic acid assembly composition comprises a lipid component and the lipid component comprises: about 50-60 mol % amine lipid such as Lipid A [or Lipid D], about 8-10 mol % neutral lipid; and about 2.5-4 mol % stealth lipid (e.g., a PEG lipid), wherein the remainder of the lipid component is helper lipid, and wherein the N/P ratio of the lipid nucleic acid assembly composition is about 6;
   (ii) the lipid nucleic acid assembly composition comprises about 50-60 mol % amine lipid such as Lipid A [or Lipid D]; about 27-39.5 mol % helper lipid; about 8-10 mol % neutral lipid; and about 2.5-4 mol % stealth lipid (e.g., a PEG lipid), wherein the N/P ratio of the lipid nucleic acid assembly composition is about 5-7 (e.g., about 6);
   (iii) the lipid nucleic acid assembly composition comprises a lipid component and the lipid component comprises: about 50-60 mol % amine lipid such as Lipid A; about 5-15 mol % neutral lipid; and about 2.5-4 mol % stealth lipid (e.g., a PEG lipid), wherein the remainder of the lipid component is helper lipid, and wherein the N/P ratio of the lipid nucleic acid assembly composition is about 3-10;
   (iv) the lipid nucleic acid assembly composition comprises a lipid component and the lipid component comprises: about 40-60 mol % amine lipid such as Lipid A [or Lipid D]; about 5-15 mol % neutral lipid; and about 2.5-4 mol % stealth lipid (e.g., a PEG lipid), wherein the remainder of the lipid component is helper lipid, and wherein the N/P ratio of the lipid nucleic acid assembly composition is about 6;
   (v) the lipid nucleic acid assembly composition comprises a lipid component and the lipid component comprises: about 50-60 mol % amine lipid such as Lipid A [or Lipid D]; about 5-15 mol % neutral lipid; and about 1.5-10 mol % stealth lipid (e.g., a PEG lipid), wherein the remainder of the lipid component is helper lipid, and wherein the N/P ratio of the lipid nucleic acid assembly composition is about 6;
   (vi) the lipid nucleic acid assembly composition comprises a lipid component and the lipid component comprises: about 40-60 mol % amine lipid such as Lipid A [or Lipid D]; about 0-10 mol % neutral lipid; and about 1.5-10 mol % stealth lipid (e.g., a PEG lipid), wherein the remainder of the lipid component is helper lipid, and wherein the N/P ratio of the lipid nucleic acid assembly composition is about 3-10;
   (vii) the lipid nucleic acid assembly composition comprises a lipid component and the lipid component comprises: about 40-60 mol % amine lipid such as Lipid A; less than about 1 mol % neutral lipid; and about 1.5-10 mol % stealth lipid (e.g., a PEG lipid), wherein the remainder of the lipid component is helper lipid, and wherein the N/P ratio of the lipid nucleic acid assembly composition is about 3-10;
   (viii) the lipid nucleic acid assembly composition comprises a lipid component and the lipid component comprises: about 40-60 mol % amine lipid such as Lipid A [or Lipid D]; and about 1.5-10 mol % Stealth lipid (e.g., a PEG lipid), wherein the remainder of the lipid component is helper lipid, wherein the N/P ratio of the LNP composition is about 3-10, and wherein the lipid nucleic acid assembly composition is essentially free of or free of neutral phospholipid;
   (ix) the lipid nucleic acid assembly composition comprises a lipid component and the lipid component comprises: about 50-60 mol % amine lipid such as Lipid A [or Lipid D]; about 8-10 mol % neutral lipid; and about 2.5-4 mol % stealth lipid (e.g., a PEG lipid), wherein the remainder of the lipid component is helper lipid, and wherein the N/P ratio of the lipid nucleic acid assembly composition is about 3-7;
   (x) the lipid nucleic acid assembly composition comprises a lipid component and the lipid component comprises: about 25-45 mol % amine lipid such as Lipid A; about 10-30 mol % neutral lipid; about 25-65 mol % helper lipid; and about 1.5-3.5 mol-% stealth lipid (e.g., a PEG lipid);
   (xi) the lipid nucleic acid assembly composition comprises a lipid component and the lipid component comprises: about 29-44 mol % amine lipid such as Lipid A; about 11-28 mol % neutral lipid; about 28-55 mol % helper lipid; and about 2.3-3.5 mol-% stealth lipid (e.g., a PEG lipid);
   (xii) the lipid nucleic acid assembly composition comprises a lipid component and the lipid component comprises: about 29-38 mol % amine lipid such as Lipid A; about 11-20 mol % neutral lipid; about 43-55 mol % helper lipid; and about 2.3-2.7 mol % stealth lipid (e.g., a PEG lipid);
   (xiii) the lipid nucleic acid assembly composition comprises a lipid component and the lipid component comprises: about 25-34 mol % amine lipid such as Lipid A; about 10-20 mol % neutral lipid; about 45-65 mol % helper lipid; and about 2.5-3.5 mol % stealth lipid (e.g., a PEG lipid);
   (xiv) the lipid nucleic acid assembly composition comprises a lipid component and the lipid component comprises: about 30-43 mol % amine lipid such as Lipid A; about 10-17 mol % neutral lipid; about 43.5-56 mol % helper lipid; and about 1.3-3 mol % stealth lipid (e.g., a PEG lipid);
   (xv) the lipid nucleic acid assembly composition comprises a lipid component and the lipid component comprises: about 25-50 mol % amine lipid such as Lipid D; about 7-25 mol % neutral lipid; about 39-65 mol % helper lipid; and about 0.5-1.8 mol % stealth lipid (e.g., a PEG lipid);
   (xvi) the lipid nucleic acid assembly composition comprises a lipid component and the lipid component comprises: about 27-40 mol % amine lipid such as Lipid D; about 10-20 mol % neutral lipid; about 50-60 mol % helper lipid; and about 0.9-1.6 mol % stealth lipid (e.g., a PEG lipid); or
   (xvii) the lipid nucleic acid assembly composition comprises a lipid component and the lipid component comprises: about 30-45 mol % amine lipid such as Lipid D; about 10-15 mol % neutral lipid; about 39-59 mol % helper lipid; and about 1-1.5 mol % stealth lipid (e.g., a PEG lipid).
Embodiment_A 57. The method of any one of the preceding embodiments_A, wherein the amine lipid is Lipid A or Lipid D.
Embodiment_A 58. The method of any one of the preceding embodiments_A, wherein the LNP has a diameter of about 1-250 nm, 10-200 nm, about 20-150 nm, about 50-150 nm, about 50-100 nm, about 50-120 nm, about 60-100 nm, about 75-150 nm, about 75-120 nm, or about 75-100 nm; or wherein the LNP has a diameter of less than 100 nm.
Embodiment_A 59. The method of any one of the preceding embodiments_A, wherein the LNP composition comprises a population of the LNP with an average diameter of about 10-200 nm, about 20-150 nm, about 50-150 nm, about 50-100 nm, about 50-120 nm, about 60-100 nm, about 75-150 nm, about 75-120 nm, or about 75-100 nm, or wherein the population of the LNP with an average diameter of less than 100 nm.
Embodiment_A 60. The method of any one of the preceding embodiments_A, wherein the lipid nucleic acid assembly composition comprises:
   a. about 50-60 mol-% amine lipid;
   b. about 5-15 mol-% DSPC; and
   c. about 2.5-4 mol-% PEG lipid,
      wherein the remainder of the lipid component is cholesterol, and
      wherein the N/P ratio of the LNP composition is 3-8±0.2.
Embodiment_A 61. The method of any one of the preceding embodiments_A wherein the average diameter is a Z-average diameter.
Embodiment_A 62. The method of any one of the preceding embodiments_A, wherein the genetically modified cell:
   a. comprises a genetic modification to decrease expression of a gene;
   b. comprises a genetic modification comprising insertion of a donor nucleic acid;
   c. exhibits increased secretion of cytokines (IL-2, IFNγ, and/or TNFα);
   d. exhibits increased cytotoxicity;
   e. exhibits increased memory cell phenotype;
   f. exhibits increased expansion;
   g. exhibits longer duration of proliferation to repeated stimulation; and/or
   h. exhibits decreased translocation events;
   optionally wherein the properties are relative to genetically modified cells made by methods other than the claimed methods.
Embodiment_A 63. The method of any one of the preceding embodiments_A, wherein the contacted cell exhibits increased survival, wherein increased survival is a post-transfection cell survival rate of at least 70%, for example at 24 hours after the last contact with an LNP composition.
Embodiment_A 64. The method of any one of the preceding embodiments_A, wherein the contacted cell exhibits increased survival, wherein increased survival is a post-transfection cell survival rate of at least 80%, for example at 24 hours after the last contact with an LNP composition.
Embodiment_A 65. The method of any one of the preceding embodiments_A, wherein the contacted cell exhibits increased survival, wherein increased survival is a post-transfection cell survival rate of at least 90%, for example at 24 hours after the last contact with an LNP composition.
Embodiment_A 66. The method of any one of the preceding embodiments_A, wherein the contacted cell exhibits increased survival, wherein increased survival is a post-transfection cell survival rate of at least 95%, for example at 24 hours after the last contact with an LNP composition.
Embodiment_A 67. The method of any one of the preceding embodiments_A, wherein the contacted cell has fewer than 1% translocations, fewer than 0.5% translocations, fewer than 0.1% translocations, or fewer than twice the background number of translocations post-editing, for example, when the translocation is a target-to-target translocation.
Embodiment_A 68. The method of any one of the preceding embodiments_A, wherein the genetically modified cell is for use in cancer therapy, or optionally autoimmune therapy.
Embodiment_A 69. An immune cell which has been genetically modified, obtainable using the method of any one of the preceding embodiments_A.
Embodiment_A 70. A composition, comprising the cell of embodiment_A 69.
Embodiment_A 71. A method of therapy, comprising administering to a patient the cell according to embodiment_A 69 or a composition according to embodiment_A70.
Embodiment_A 72. A method of therapy according to embodiment_A71, for treatment of cancer, or optionally autoimmune therapy.
Embodiment_A 73. The method of embodiment_A72, wherein the cell expresses a TCR with specificity for a polypeptide expressed by cells of the cancer.
Embodiment_A 74. A cell or population of cells produced by the method of any one of embodiments_A 1 to 159 for use as an ACT therapy, wherein the cell or population of cells has low toxicity, i.e., the method used to make the cell or population of cells has a low level of toxicity to the cells resulting in a cell or cells that have a high level of viability.
Embodiment_A 75. A cell or population of cells produced by the method of any one of embodiments_A 1 to 67 for use as an ACT therapy, wherein the cell or population of cells has fewer than 2% translocations, fewer than 1% translocations, fewer than 0.5% translocations, or fewer than 0.1% translocations, e.g., target-to-target translocations; or fewer than twice the background level of translocations.

The following non-limiting embodiments are also encompassed:
Embodiment_B 1. A method of producing a population of B cells comprising edited B cells, comprising culturing a population of B cells in vitro and contacting the population with one or more lipid nanoparticles (LNPs) comprising a genome editing tool.
Embodiment_B 2. A method of producing a population of B cells comprising edited B cells, comprising culturing a population of B cells in vitro and contacting the population with i) one or more lipid nanoparticles (LNPs) comprising a genome editing tool; and ii) a DNA-PK inhibitor.
Embodiment_B 3. The method of any one of the preceding embodiments_B, wherein the edited B cells comprise multiple genome edits per cell.
Embodiment_B 4. The method of any one of the preceding embodiments_B, further comprising activating the population of B cells prior to the contacting step.
Embodiment_B 5. A method of producing a population of B cells comprising edited B cells, comprising culturing a population of B cells in vitro and activating the B cells prior to contacting the population with one or more lipid nanoparticles (LNPs) comprising a genome editing tool, wherein the population is contacted with the one or more LNP on the same day as activation or up to 10 days after activation.
Embodiment_B 6. A method of producing a population of B cells comprising edited B cells, comprising the steps of:
   a. culturing a population of B cells in vitro;
   b. activating the population of B cells in vitro;
   c. contacting the population of B cells of b) in vitro with one or more lipid nanoparticles (LNPs), wherein the LNP comprises a genome editing tool; and
   d. contacting the population of B cells with a DNA-PK inhibitor;
      thereby producing a population of edited B cells.
Embodiment_B 7. The method of any one embodiments_B 5 or 6, wherein the edited B cells comprise multiple genome edits per cell.
Embodiment_B 8. The method of any of the preceding embodiments_B, wherein the population of B cells is activated, and wherein the population of B cells are activated using an agent comprising CD40L.
Embodiment_B 9. The method of any of the preceding embodiments_B, wherein the population of B cells is activated, and wherein the population of B cells are activated with CpG.
Embodiment_B 10. The method of any of the preceding embodiments_B, wherein the population of B cells is activated, and wherein the population of B cells are activated in media comprising human serum.
Embodiment_B 11. The method of any of the preceding embodiments_B, wherein the population of B cells is activated, and wherein the population of B cells is contacted in vitro on the same day or up to 10 days after activation with the one or more LNPs.
Embodiment_B 12. The method of any of the preceding embodiments_B, wherein the population of B cells is activated, and wherein the population of B cells is contacted in vitro on the same day as activation with the one or more LNPs.
Embodiment_B 13. The method of any of the preceding embodiments_B, wherein the population of B cells is activated, and wherein the population of B cells is contacted in vitro 1 day after activation with the one or more LNPs.
Embodiment_B 14. The method of any of the preceding embodiments_B, wherein the population of B cells is activated, and wherein the population of B cells is contacted in vitro 2 days after activation with the one or more LNPs.
Embodiment_B 15. The method of any of the preceding embodiments_B, wherein the population of B cells is activated, and wherein the population of B cells is contacted in vitro 3 days after activation with the one or more LNPs.
Embodiment_B 16. The method of any of the preceding embodiments_B, wherein the population of B cells is activated, and wherein the population of B cells is contacted in vitro 4 days after activation with the one or more LNPs.
Embodiment_B 17. The method of any of the preceding embodiments_B, wherein the population of B cells is activated, and wherein the population of B cells is contacted in vitro 5 days after activation with the one or more LNPs.
Embodiment_B 18. The method of any of the preceding embodiments_B, wherein the population of B cells is activated, and wherein the population of B cells is contacted in vitro 6 days after activation with the one or more LNPs.
Embodiment_B 19. The method of any of the preceding embodiments_B, wherein the population of B cells is activated, and wherein the population of B cells is contacted in vitro 7 days after activation with the one or more LNPs.
Embodiment_B 20. The method of any of the preceding embodiments_B, wherein the population of B cells is activated, and wherein the population of B cells is contacted in vitro 8 days after activation with the one or more LNPs.
Embodiment_B 21. The method of any of the preceding embodiments_B, wherein the population of B cells is activated, and wherein the population of B cells is contacted in vitro 9 days after activation with the one or more LNPs.
Embodiment_B 22. The method of any of the preceding embodiments_B, wherein the population of B cells is activated, and wherein the population of B cells is contacted in vitro 10 days after activation with the one or more LNPs.
Embodiment_B 23. The method of any of the preceding embodiments_B, wherein the LNPs are preincubated with ApoE prior contacting the population of B cells with the LNPs.
Embodiment_B 24. The method of any of the preceding embodiments_B, wherein the LNPs are preincubated with ApoE3 prior contacting the population of B cells with the LNPs.
Embodiment_B 25. The method of any of the preceding embodiments_B, wherein the LNPs are preincubated with ApoE4 prior contacting the population of B cells with the LNPs.
Embodiment_B 26. The method of any of the preceding embodiments_B, wherein the population of B cells are contacted with LNPs comprising 2.5-10 μg/mL total RNA cargo.
Embodiment_B 27. The method of any of the preceding embodiments_B, wherein the population of B cells is contacted with 2-10 LNPs, e.g., two lipid nanoparticles (LNPs).
Embodiment_B 28. The method of any of the preceding embodiments_B, wherein the population of B cells is contacted with three lipid nanoparticles (LNPs).
Embodiment_B 29. The method of any of the preceding embodiments_B, wherein the population of B cells is contacted with four lipid nanoparticles (LNPs).
Embodiment_B 30. The method of any of the preceding embodiments_B, wherein the population of B cells is contacted with five lipid nanoparticles (LNPs).
Embodiment_B 31. The method of any of the preceding embodiments_B, wherein the population of B cells is contacted with six lipid nanoparticles (LNPs).
Embodiment_B 32. The method of any of the preceding embodiments_B, further comprising contacting the population of B cells with a donor nucleic acid for insertion into a target sequence.
Embodiment_B 33. The method of any of the preceding embodiments_B, wherein the method produces a population of B cells comprising at least 20%, 30%, 40%, 50%, 60%, 70%, or 80% of cells comprising a genome edit.
Embodiment_B 34. The method of embodiment_B 33, wherein the genome edit comprises an indel or a base edit and the population of B cells comprises at least 40%, 50%, 60%, 70%, or 80% of cells comprising a genome edit.
Embodiment_B 35. The method embodiments_B 33 or 34, wherein the genome edit comprises an insertion of an exogenous nucleic acid sequence into a target sequence and the population of B cells comprises at least 20%, 30%, or 40% of cells comprising a genome edit.
Embodiment_B 36. The method of embodiments_B 33-35, wherein the population of cells comprises edited B cells comprising at least two genome edits, wherein at least 20%, 30%, 40%, 50%, or 60% of cells comprise both genome edits.
Embodiment_B 37. The method of any of the preceding embodiments_B, wherein the method produces a population of B cells comprising edited B cells comprising multiple genome edits per cell, wherein fewer than 1% of the cells have a target-to-target translocations.
Embodiment_B 38. The method of any of the preceding embodiments_B, wherein the method produces a population of B cells comprising edited B cells comprising multiple genome edits per cell, wherein fewer than 0.5% of the cells have a target-to-target translocations.
Embodiment_B 39. The method of any of the preceding embodiments_B, wherein the method produces a population of B cells comprising edited B cells comprising multiple genome edits per cell, wherein fewer than 0.2% of the cells have a target-to-target translocations.
Embodiment_B 40. The method of any of the preceding embodiments_B, wherein the method produces a population of B cells comprising edited B cells comprising multiple genome edits per cell, wherein fewer than 0.1% of the cells have a target-to-target translocations.
Embodiment_B 41. The method of any of the preceding embodiments_B, wherein the method produces a population of B cells comprising edited B cells comprising multiple genome edits per cell, wherein the edited cells have less than 2 times the background level of reciprocal translocations, complex translocations, or off-target translocations.
Embodiment_B 42. The method of any of the preceding embodiments_B, wherein the edited B cells comprise memory B cells.
Embodiment_B 43. The method of any of the preceding embodiments_B, wherein the edited B cells comprise plasma blasts.
Embodiment_B 44. The method of any of the preceding embodiments_B, wherein the edited B cells comprise plasma cells.
Embodiment_B 45. The method of any of the preceding embodiments_B, wherein one of the LNP compositions comprises a gRNA targeting a gene that reduces surface expression of MHC class I.
Embodiment_B 46. The method of any of the preceding embodiments_B, wherein one of the LNP compositions comprises a gRNA targeting B2M.
Embodiment_B 47. The method of any of the preceding embodiments_B, wherein the LNP composition comprises a guide RNA and an mRNA encoding the RNA guided-DNA binding agent, such as a Cas9, optionally an S. pyogenes Cas9.
Embodiment_B 48. The method of any of the preceding embodiments_B, wherein the LNP composition comprises a guide RNA and an mRNA encoding the RNA guided-DNA binding agent, wherein the RNA guided-DNA binding agent is a nickase.
Embodiment_B 49. The method of any of the preceding embodiments_B, wherein the LNP composition comprises a guide RNA and an mRNA encoding the RNA guided-DNA binding agent, wherein the RNA guided-DNA binding agent is a cleavase.
Embodiment_B 50. The method of any preceding embodiments_B, wherein the method does not comprise a selection step, optionally a physical selection step or a biochemical selection step.
Embodiment_B 51. The method of any preceding embodiments_B, wherein the methods are performed ex vivo.
Embodiment_B 52. A composition comprising a population of B cells comprising edited B cells, wherein the population of B cells comprises at least 20%, 30%, 40%, 50%, 60%, 70%, 80% of cells comprising a genome edit.
Embodiment_B 53. A composition comprising a population of B cells comprising edited B cells, wherein the population of B cells comprises at least 40%, 50%, 60%, 70%, or 80% of cells comprising a genome edit, wherein the genome edit comprises an indel or a base edit.
Embodiment_B 54. A composition comprising a population of B cells comprising edited B cells, wherein the population of B cells comprises at least 20%, 30%, or 40% of cells comprising a genome edit, wherein the genome edit comprises an insertion of an exogenous nucleic acid into a target sequence.
Embodiment_B 55. A composition comprising a population of B cells comprising edited B cells, wherein the population of B cells comprises at least 20%, 30%, 40%, 50%, or 60% of cells comprising at least two genome edits.
Embodiment_B 56. The composition of any of embodiments_B52-55, wherein fewer than 1% of the cells have a target-to-target translocations.
Embodiment_B 57. The composition of any of embodiments_B52-55, wherein fewer than 0.5% of the cells have a target-to-target translocations.
Embodiment_B 58. The composition of any of embodiments_B 52-55, wherein fewer than 0.2% of the cells have a target-to-target translocations.
Embodiment_B 59. The composition of any of embodiments_B52-55, wherein fewer than fewer than 0.1% of the cells have a target-to-target translocations.
Embodiment_B 60. The composition of any of embodiments_B52-59, wherein the cells have less than 2 times the background level of reciprocal translocations, complex translocations, or off-target translocations.
Embodiment_B 61. The composition of any of embodiments_B52-60, wherein the edited B cells comprise memory B cells.
Embodiment_B 62. The composition of any of embodiments_B52-61, wherein the edited B cells comprise plasma blasts.
Embodiment_B 63. The composition of any of embodiments_B52-62, wherein the edited B cells comprise plasma cells.
Embodiment_B 64. A composition comprising a population of B cells comprising edited B cells, wherein the edited B cells are obtained by or obtainable by the method of any of embodiments_B1-51.

The following non-limiting embodiments are also encompassed:
Embodiment_C 1. A method of producing a population of NK cells comprising edited NK cells, comprising culturing a population of NK cells in vitro and contacting the population with one or more lipid nanoparticles (LNPs) comprising a genome editing tool.
Embodiment_C 2. A method of producing a population of NK cells comprising edited NK cells, comprising culturing a population of NK cells in vitro and contacting the population with i) one or more lipid nanoparticles (LNPs) comprising a genome editing tool; and ii) a DNA-PK inhibitor.
Embodiment_C 3. The method of any one of the preceding embodiments_C, wherein the edited NK cells comprise multiple genome edits per cell.
Embodiment_C 4. The method of any one of the preceding embodiments_C, further comprising activating the population of NK cells prior to the contacting step.
Embodiment_C 5. The method of any one of the preceding embodiments_C, further comprising activating the population of NK cells prior to the contacting step, wherein the population is contacted with the one or more LNP at least 3 days after activation.
Embodiment_C 6. A method of producing a population of NK cells comprising edited NK cells comprising multiple genome edits per cell, comprising the steps of:
   a. culturing a population of NK cells in vitro;
   b. activating the population of NK cells in vitro;
   c. contacting the population of NK cells of b) in vitro with one or more lipid nanoparticles (LNPs), wherein the LNP comprises a genome editing tool; and
   d. contacting the population of NK cells with a DNA-PK inhibitor;
      thereby producing a population of edited NK cells.
Embodiment_C 7. The method of embodiment_C 6, wherein the edited NK cells comprise multiple genome edits per cell.
Embodiment_C 8. The method of any of the preceding embodiments_C, wherein the population of NK cells is activated, and wherein the population of NK cells are activated with feeder cells and cytokines.
Embodiment_C 9. The method of any of the preceding embodiments_C, wherein the population of NK cells is activated, and wherein the population of NK cells are activated with feeder cells and cytokines, and wherein the ratio of NK cells to feeder cells in step a) is 1:1.
Embodiment_C 10. The method of any of the preceding embodiments_C, wherein the population of NK cells is activated, and wherein the population of NK cells are activated with feeder cells and cytokines, and wherein the cytokines include IL-2.
Embodiment_C 11. The method of any of the preceding embodiments_C, wherein the population of NK cells is activated, and wherein the population of NK cells are activated with feeder cells and cytokines, and wherein the cytokines include IL-15.
Embodiment_C 12. The method of any of the preceding embodiments_C, wherein the population of NK cells is activated, and wherein the population of NK cells are activated with feeder cells and cytokines, and wherein the cytokines include IL-21.
Embodiment_C 13. The method of any of the preceding embodiments_C, wherein the population of NK cells is activated, and wherein the population of NK cells are activated at least 3 days prior to the contacting step.
Embodiment_C 14. The method of any of the preceding embodiments_C, wherein the LNPs are preincubated with ApoE prior contacting the population of NK cells with the LNPs.
Embodiment_C 15. The method of any of the preceding embodiments_C, wherein the LNPs are preincubated with ApoE3 prior contacting the population of NK cells with the LNPs.
Embodiment_C 16. The method of any of the preceding embodiments_C, wherein the LNPs are preincubated with ApoE4 prior contacting the population of NK cells with the LNPs.
Embodiment_C 17. The method of any of the preceding embodiments_C, wherein the population of NK cells are contacted with LNPs comprising 2.5-10 μg/mL total RNA cargo.
Embodiment_C 18. The method of any of the preceding embodiments_C, wherein the population of NK cells is contacted with 2-10 LNP, e.g., two lipid nanoparticles (LNPs).
Embodiment_C 19. The method of any of the preceding embodiments_C, wherein the population of NK cells is contacted with three lipid nanoparticles (LNPs).
Embodiment_C 20. The method of any of the preceding embodiments_C, wherein the population of NK cells is contacted with four lipid nanoparticles (LNPs).
Embodiment_C 21. The method of any of the preceding embodiments_C, wherein the population of NK cells is contacted with five lipid nanoparticles (LNPs).
Embodiment_C 22. The method of any of the preceding embodiments_C, wherein the population of NK cells is contacted with six lipid nanoparticles (LNPs).
Embodiment_C 23. The method of any of the preceding embodiments_C, further comprising contacting the population of NK cells with a donor nucleic acid for insertion into a target sequence.
Embodiment_C 24. The method of any of the preceding embodiments_C, wherein the method produces a population of NK cells comprising at least 40%, 50%, 60%, 70%, 80%, 90%, or 95% of cells comprising a genome edit.
Embodiment_C 25. The method of embodiment_C 24, wherein the genome edit comprises an indel or a base edit and the population of NK cells comprises at least 40%, 50%, 60%, 70%, 80%, 90%, or 95% of cells comprising a genome edit.
Embodiment_C 26. The method of embodiments_C 24 or 25, wherein the genome edit comprises an insertion of as exogenous nucleic acid sequence into a target sequence and the population of NK cells comprises at least 40%, 50%, 60%, 70%, 80%, 90%, or 95% of cells comprising a genome edit.
Embodiment_C 27. The method of embodiments_C 24-26, wherein the population of cells comprises edited NK cells comprising at least two genome edits, wherein at least 40%, 50%, 60%, 70%, or 80% of cells comprise both genome edits.
Embodiment_C 28. The method of any of the preceding embodiments_C, wherein the method produces a population of NK cells comprising edited NK cells comprising multiple genome edits per cell, wherein fewer than 1% of the cells have a target-to-target translocations.
Embodiment_C 29. The method of any of the preceding embodiments_C, wherein the method produces a population of NK cells comprising edited NK cells comprising multiple genome edits per cell, wherein fewer than 0.5% of the cells have a target-to-target translocations.
Embodiment_C 30. The method of any of the preceding embodiments_C, wherein the method produces a population of NK cells comprising edited NK cells comprising multiple genome edits per cell, wherein fewer than 0.2% of the cells have a target-to-target translocations.
Embodiment_C 31. The method of any of the preceding embodiments, embodiments_C, wherein the method produces a population of NK cells comprising edited NK cells comprising multiple genome edits per cell, wherein fewer than 0.1% of the cells have a target-to-target translocations.
Embodiment_C 32. The method of any of the preceding embodiments, embodiments_C, wherein the method produces a population of NK cells comprising edited NK cells comprising multiple genome edits per cell, wherein the edited cells have less than 2 times the background level of reciprocal translocations, complex translocations, or off-target translocations.
Embodiment_C 33. The method of any of the preceding embodiments_C, wherein the LNP composition comprises a guide RNA and an mRNA encoding the RNA guided-DNA binding agent, such as a Cas9, optionally an S. pyogenes Cas9
Embodiment_C 34. The method of any of the preceding embodiments_C, wherein the LNP composition comprises a guide RNA and an mRNA encoding the RNA guided-DNA binding agent, wherein the RNA guided-DNA binding agent is a nickase.
Embodiment_C 35. The method of any of the preceding embodiments_C, wherein the LNP composition comprises a guide RNA and an mRNA encoding the RNA guided-DNA binding agent, wherein the RNA guided-DNA binding agent is a cleavase.
Embodiment_C 36. The method of any preceding embodiments_C, wherein the methods are performed ex vivo.
Embodiment_C 37. A composition comprising a population of NK cells comprising edited NK cells, wherein the population of NK cells comprises at least 40%, 50%, 60%, 70%, 80%, 90%, or 95% of cells comprising a genome edit.
Embodiment_C 38. A composition comprising a population of NK cells comprising edited NK cells, wherein the population of NK cells comprises at least 40%, 50%, 60%, 70%, 80%, 90%, or 95% of cells comprising a genome edit, wherein the genome edit comprises an indel or a base edit.
Embodiment_C 39. A composition comprising a population of NK cells comprising edited NK cells, wherein the population of NK cells comprises at least 40%, 50%, 60%, 70%, 80%, 90%, or 95% of cells comprising a genome edit, wherein the genome edit comprises an insertion of an exogenous nucleic acid into a target sequence.
Embodiment_C 40. A composition comprising a population of NK cells comprising edited NK cells, wherein the population of NK cells comprises at least 40%, 50%, 60%, 70%, or 80% of cells comprising at least two genome edits.
Embodiment_C 41. The composition of any of embodiments_C37-40, wherein fewer than 1% of the cells have a target-to-target translocations.
Embodiment_C 42. The composition of any of embodiments_C37-40, wherein fewer than 0.5% of the cells have a target-to-target translocations.
Embodiment_C 43. The composition of any of embodiments_C37-40, wherein fewer than 0.2% of the cells have a target-to-target translocations.
Embodiment_C 44. The composition of any of embodiments_C37-40, wherein fewer than 0.1% of the cells have a target-to-target translocations.
Embodiment_C 45. The composition of any of embodiments_C37-44, wherein the cells have less than 2 times the background level of reciprocal translocations, complex translocations, or off-target translocations.
Embodiment_C 46. A composition comprising a population of NK cells comprising edited NK cells, wherein the edited NK cells are obtained by or obtainable by the method of any of embodiments_C1-C₃₆.

The following non-limiting embodiments are also encompassed:
Embodiment_D 1. A method of producing a population of monocytes comprising edited cells, comprising culturing a population of monocytes in vitro and contacting the population with one or more lipid nanoparticles (LNPs) comprising a genome editing tool.
Embodiment_D 2. A method of producing a population of monocytes comprising edited cells, comprising culturing a population of monocytes in vitro and contacting the population with i) one or more lipid nanoparticles (LNPs) comprising a genome editing tool; and ii) a DNA-PK inhibitor.
Embodiment_D 3. The method of any one of the preceding embodiments_D, wherein the edited cells comprise multiple genome edits per cell.
Embodiment_D 4. The method of any one of the preceding embodiments_D, further comprising differentiating the population of monocytes cells prior to the contacting step.
Embodiment_D 5. A method of producing a population of monocytes comprising edited cells, comprising culturing a population of monocytes in vitro and differentiating the population of monocytes prior to contacting the population with one or more lipid nanoparticles (LNPs) comprising a genome editing tool, wherein the population of monocytes is differentiated for 0-8 days prior to contact with the one or more LNPs.
Embodiment_D 6. A method of producing a population of monocytes comprising edited cells, comprising the steps of:
   a. culturing a population of monocytes in vitro;
   b. differentiating the monocytes in vitro;
   c. contacting the population of cells of b) in vitro with one or more lipid nanoparticles (LNPs), wherein the LNP comprises a genome editing tool; and
   d. contacting the population of cells of b) with a DNA-PK inhibitor;

thereby producing a population of edited cells.
Embodiment_D 7. The method of any one embodiments_D 5 or 6, wherein the edited cells comprise multiple genome edits per cell.
Embodiment_D 8. The method of any of the preceding embodiments_D, wherein the population of monocytes are differentiated with GM-CSF.
Embodiment_D 9. The method of any of the preceding embodiments_D, wherein the monocytes are differentiated for 0-8 days prior to the contacting step.
Embodiment_D 10. The method of any of the preceding embodiments_D, wherein the monocytes are differentiated for 0-5 days prior to the contacting step.
Embodiment_D 11. The method of any of the preceding embodiments_D, wherein the monocytes are differentiated for 0 days prior to the contacting step.
Embodiment_D 12. The method of any of the preceding embodiments_D, wherein the monocytes are differentiated for 1 day prior to the contacting step.
Embodiment_D 13. The method of any of the preceding embodiments_D, wherein the monocytes are differentiated for 2 days prior to the contacting step.
Embodiment_D 14. The method of any of the preceding embodiments_D, wherein the monocytes are differentiated for 3 days prior to the contacting step.
Embodiment_D 15. The method of any of the preceding embodiments_D, wherein the monocytes are differentiated for 4 days prior to the contacting step.
Embodiment_D 16. The method of any of the preceding embodiments_D, wherein the monocytes are differentiated for 5 days prior to the contacting step.
Embodiment_D 17. The method of any of the preceding embodiments_D, wherein the monocytes are differentiated for 6 days prior to the contacting step.
Embodiment_D 18. The method of any of the preceding embodiments_D, wherein the monocytes are differentiated for 7 days prior to the contacting step.
Embodiment_D 19. The method of any of the preceding embodiments_D, wherein the monocytes are differentiated for 8 days prior to the contacting step.
Embodiment_D 20. The method of any of the preceding embodiments_D, wherein the LNPs are preincubated with ApoE prior contacting the population of monocytes or macrophages with the LNPs.
Embodiment_D 21. The method of any of the preceding embodiments_D, wherein the LNPs are preincubated with ApoE3 prior contacting the population of monocytes with the LNPs.
Embodiment_D 22. The method of any of the preceding embodiments_D, wherein the LNPs are preincubated with ApoE4 prior contacting the population of monocytes with the LNPs.
Embodiment_D 23. The method of any of the preceding embodiments_D, wherein the LNPs are preincubated with serum prior contacting the population of monocytes with the LNPs.
Embodiment_D 24. The method of any of the preceding embodiments_D, wherein the monocytes are cultured in media comprising serum prior to and/or during the contacting step.
Embodiment_D 25. The method of any of the preceding embodiments_D, wherein the population of monocytes are contacted with LNPs comprising 2.5-10 μg/mL total RNA cargo.
Embodiment_D 26. The method of any of the preceding embodiments_D, wherein the population of monocytes is contacted with two lipid nanoparticles (LNPs).
Embodiment_D 27. The method of any of the preceding embodiments_D, wherein the population of monocytes is contacted with three lipid nanoparticles (LNPs).
Embodiment_D 28. The method of any of the preceding embodiments_D, wherein the population of monocytes is contacted with four lipid nanoparticles (LNPs).
Embodiment_D 29. The method of any of the preceding embodiments_D, wherein the population of monocytes is contacted with five lipid nanoparticles (LNPs).
Embodiment_D 30. The method of any of the preceding embodiments_D, wherein the population of monocytes is contacted with six lipid nanoparticles (LNPs).
Embodiment_D 31. The method of any of the preceding embodiments_D, further comprising contacting the population of monocytes with a donor nucleic acid for insertion into a target sequence.
Embodiment_D 32. The method of any of the preceding embodiments_D, wherein the method produces a population of monocytes comprising edited cells comprising at least 50%, 60%, 70%, 80%, 90%, 95%, or 96% of cells comprising a genome edit.
Embodiment_D 33. The method of embodiment_D 32, wherein the genome edit comprises an indel or a base edit and the population of monocytes comprises at least 40%, 50%, 60%, 70%, 80%, 90%, or 95% of cells comprising a genome edit.
Embodiment_D 34. The method embodiments_D 32 or 33, wherein the genome edit comprises an insertion of an exogenous nucleic acid sequence into a target sequence and the population of monocytes comprises at least 40%, 50%, 60%, 70%, 80%, 90%, or 95% of cells comprising a genome edit.
Embodiment_D 35. The method of embodiments_D 32-34, wherein the population of cells comprises edited cells comprising at least two genome edits, wherein at least 40%, 50%, 60%, 70%, or 80% of cells comprise both genome edits.
Embodiment_D 36. The method of any of the preceding embodiments_D, wherein the method produces a population of monocytes comprising edited cells comprising multiple genome edits per cell, wherein fewer than 1% of the cells have a target-to-target translocations.
Embodiment_D 37. The method of any of the preceding embodiments_D, wherein the method produces a population of monocytes comprising cells comprising multiple genome edits per cell, wherein fewer than 0.5% of the cells have a target-to-target translocations.
Embodiment_D 38. The method of any of the preceding embodiments_D, wherein the method produces a population of monocytes comprising cells comprising multiple genome edits per cell, wherein fewer than 0.2% of the cells have a target-to-target translocations.
Embodiment_D 39. The method of any of the preceding embodiments_D, wherein the method produces a population of monocytes comprising cells comprising multiple genome edits per cell, wherein fewer than 0.1% of the cells have a target-to-target translocations.
Embodiment_D 40. The method of any of the preceding embodiments_D, wherein the method produces a population of monocytes comprising cells comprising multiple genome edits per cell, wherein the cells have less than 2 times the background level of reciprocal translocations, complex translocations, or off-target translocations.
Embodiment_D 41. The method of any of the preceding embodiments_D, wherein one of the LNP compositions comprises a gRNA targeting a gene that reduces or eliminates surface expression of MHC class I.
Embodiment_D 42. The method of any of the preceding embodiments_D, wherein one of the LNP compositions comprises a gRNA targeting B2M.
Embodiment_D 43. The method of any of the preceding embodiments_D, wherein one of the LNP compositions comprises a gRNA targeting a gene that reduces or eliminates surface expression of MHC class II.
Embodiment_D 44. The method of any of the preceding embodiments_D, wherein one of the LNP compositions comprises a gRNA targeting CIITA.
Embodiment_D 45. The method of any of the preceding embodiments_D, wherein the LNP composition comprises a guide RNA and an mRNA encoding the RNA guided-DNA binding agent, such as a Cas9, optionally an S. pyogenes Cas9
Embodiment_D 46. The method of any of the preceding embodiments_D, wherein the LNP composition comprises a guide RNA and an mRNA encoding the RNA guided-DNA binding agent, wherein the RNA guided-DNA binding agent is a nickase.
Embodiment_D 47. The method of any of the preceding embodiments_D, wherein the LNP composition comprises a guide RNA and an mRNA encoding the RNA guided-DNA binding agent, wherein the RNA guided-DNA binding agent is a cleavase.
Embodiment_D 48. The method of any preceding embodiments_D, wherein the methods are performed ex vivo.
Embodiment_D 49. A composition comprising a population of monocytes comprising edited cells, wherein the population of monocytes comprises at least 40%, 50%, 60%, 70%, 80%, 90%, or 95% of cells comprising a genome edit.
Embodiment_D 50. A composition comprising a population of monocytes comprising edited cells, wherein the population of monocytes comprises at least 40%, 50%, 60%, 70%, 80%, 90%, or 95% of cells comprising a genome edit, wherein the genome edit comprises an indel or a base edit.
Embodiment_D 51. A composition comprising a population of monocytes comprising edited cells, wherein the population of monocytes comprises at least 40%, 50%, 60%, 70%, 80%, 90%, or 95% of cells comprising a genome edit, wherein the genome edit comprises an insertion of an exogenous nucleic acid sequence into a target sequence.
Embodiment_D 52. A composition comprising a population of monocytes comprising edited cells, wherein the population of monocytes comprises at least 40%, 50%, 60%, 70%, or 80% of cells comprising at least two genome edits.
Embodiment_D 53. The composition of any of embodiments_D49-52, wherein fewer than 1% of the cells have a target-to-target translocations.
Embodiment_D 54. The composition of any of embodiments_D49-52, wherein fewer than 0.5% of the cells have a target-to-target translocations.
Embodiment_D 55. The composition of any of embodiments_D49-52, wherein fewer than 0.2% of the cells have a target-to-target translocations.
Embodiment_D 56. The composition of any of embodiments_D49-55, wherein fewer than 0.1% of the cells have a target-to-target translocations.
Embodiment_D 57. The composition of any of embodiments_D49-56, wherein the cells have less than 2 times the background level of reciprocal translocations, complex translocations, or off-target translocations.
Embodiment_D 58. The composition of any of embodiments_D49-57, wherein the edited cells comprise macrophages.
Embodiment_D 59. A composition comprising a population of monocytes or macrophages comprising edited monocytes or macrophages, wherein the edited monocytes or macrophages are obtained by or obtainable by the method of any of embodiments_D1-48.

The following non-limiting embodiments are also encompassed:
Embodiment_E 1. A method of producing multiple genome edits in an in vitro-cultured iPSC, comprising the steps of:
   a. contacting the iPSC in vitro with at least first lipid nanoparticle (LNP) composition and second LNP composition, wherein the first lipid nucleic acid assembly composition comprises a first guide RNA (gRNA) directed to a first target sequence and optionally a nucleic acid genome editing tool and the second lipid nucleic acid assembly composition comprises a second gRNA directed to a second target sequence different from the first target sequence and optionally a nucleic acid genome editing tool; and
   b. optionally, expanding the cell in vitro;
      thereby producing multiple genome edits in the cell.
Embodiment_E 2. The method of embodiment_E 1, wherein the iPSC is expanded in vitro.
Embodiment_E 3. The method of embodiments_E 1 or 2, wherein the method is performed on a population of iPSC.
Embodiment_E 4. A method of producing a population of iPSC comprising edited iPSCs comprising a genome edit comprising culturing a population of iPSC in vitro with one or more lipid nanoparticles (LNP) comprising a genome editing tool.
Embodiment_E 5. A method of producing a population of iPSC comprising edited iPSCs comprising a genome edit comprising culturing a population of iPSC in vitro with (i) one or more lipid nanoparticles (LNP) comprising a genome editing tool and (ii) a DNA-PK inhibitor.
Embodiment_E 6. A method of producing a population of iPSC comprising edited iPSCs comprising multiple genome edits comprising culturing a population of iPSC in vitro with (i) two or more lipid nanoparticles (LNP) comprising a genome editing tool and (ii) a DNA-PK inhibitor.
Embodiment_E 7. The method of any one of the preceding embodiments_E, further comprising identifying an edited iPSC in a population of iPSC.
Embodiment_E 8. The method of any one of the preceding embodiments_E, further comprising isolating an edited iPSC.
Embodiment_E 9. The method of embodiment_E 8, further comprising expanding the isolated cell in vitro.
Embodiment_E 10. The method of any one of the preceding embodiments_E, comprising contacting the cell in vitro with up to 10 LNPs.
Embodiment_E 11. The method of any one of the preceding embodiments_E, wherein the method produces a population of cells comprising edited cells comprising multiple genome edits per cell, wherein fewer than 1% of the cells have a target-to-target translocations.
Embodiment_E 12. The method of any one of the preceding embodiments_E, wherein the method produces a population of cells comprising edited cells comprising multiple genome edits per cell, wherein fewer than 0.5% of the cells have a target-to-target translocations.
Embodiment_E 13. The method of any one of the preceding embodiments_E, wherein the method produces a population of cells comprising edited cells comprising multiple genome edits per cell, wherein fewer than 0.2% of the cells have a target-to-target translocations.
Embodiment_E 14. The method of any one of the preceding embodiments_E, wherein the method produces a population of cells comprising edited cells comprising multiple genome edits per cell, wherein fewer than 0.1% of the cells have a target-to-target translocations.
Embodiment_E 15. The method of any of the preceding embodiments_E, wherein the method produces a population of cells comprising edited cells comprising multiple genome edits per cell, wherein the cells have less than 2 times the background level of reciprocal translocations, complex translocations, or off-target translocations.
Embodiment_E 16. The method of any of the preceding embodiments_E, wherein the method produces a population of cells comprising edited cells comprising at least 20%, 30%, 40%, or 50% of cells comprising a genome edit.
Embodiment_E 17. The method of any of the preceding embodiments_E, wherein the LNP composition comprises a guide RNA and an mRNA encoding the RNA guided-DNA binding agent, such as a Cas9, optionally an S. pyogenes Cas9.
Embodiment_E 18. The method of any of the preceding embodiments_E, wherein the LNP composition comprises a guide RNA and an mRNA encoding the RNA guided-DNA binding agent, wherein the RNA guided-DNA binding agent is a nickase.
Embodiment_E 19. The method of any of the preceding embodiments_E, wherein the LNP composition comprises a guide RNA and an mRNA encoding the RNA guided-DNA binding agent, wherein the RNA guided-DNA binding agent is a cleavase.
Embodiment_E 20. The composition of any of the preceding embodiments_E, wherein the population of cells is further contacted with a DNA-PK inhibitor.
Embodiment_E 21. The composition of any of the preceding embodiments_E, wherein the cells are human cells.
Embodiment_E 22. The method of any preceding embodiments_E, wherein the methods are performed ex vivo.
Embodiment_E 23. The method of any of the preceding embodiments_E, wherein one of the LNP compositions comprises a gRNA targeting a gene that reduces or eliminates surface expression of MHC class I.
Embodiment_E 24. The method of any of the preceding embodiments_E, wherein one of the LNP compositions comprises a gRNA targeting B2M.
Embodiment_E 25. The method of any of the preceding embodiments_E, wherein one of the LNP compositions comprises a gRNA targeting a gene that reduces or eliminates surface expression of MHC class II.
Embodiment_E 26. The method of any of the preceding embodiments_E, wherein one of the LNP compositions comprises a gRNA targeting CIITA.
Embodiment_E 27. A composition comprising a population of iPSC comprising edited cells, wherein the population comprises at least 20%, 30%, 40%, or 50% of cells comprising a genome edit.
Embodiment_E 28. The composition of embodiment_E27, wherein fewer than 1% of the cells have a target-to-target translocations.
Embodiment_E 29. The composition of embodiment_E27, wherein fewer than 0.5% of the cells have a target-to-target translocations.
Embodiment_E 30. The composition of embodiment_E27, wherein fewer than 0.2% of the cells have a target-to-target translocations.
Embodiment_E 31. The composition of embodiment_E27, wherein fewer than 0.1% of the cells have a target-to-target translocations.
Embodiment_E 32. The composition of any of embodiments_E27-31, wherein the cells have less than 2 times the background level of reciprocal translocations, complex translocations, or off-target translocations.
Embodiment_E 33. A composition comprising a population of iPSCs comprising edited iPSCs, wherein the edited iPSCs are obtained by or obtainable by the method of any of embodiments_E1-26.

The following non-limiting embodiments are also encompassed:
Embodiment_F 1. A method of producing a population of T cells comprising edited T cells, comprising culturing a population of T cells in vitro and contacting the population with one or more lipid nanoparticles (LNPs) comprising a genome editing tool.
Embodiment_F 2. A method of producing a population of B cells comprising edited T cells, comprising culturing a population of T cells in vitro and contacting the population with i) one or more lipid nanoparticles (LNPs) comprising a genome editing tool; and ii) a DNA-PK inhibitor.
Embodiment_F 3. The method of any one of the preceding embodiments_F, wherein the edited T cells comprise multiple genome edits per cell.
Embodiment_F 4. The method of any one of the preceding embodiments_F, further comprising activating the population of T cells prior to the contacting step.
Embodiment_F 5. A method of producing a population of T cells comprising edited T cells, comprising the steps of:
   a. culturing a population of T cells in vitro;
   b. activating the population of T cells in vitro;
   c. contacting the population of T cells of b) in vitro with one or more lipid nanoparticles (LNPs), wherein the LNP comprises a genome editing tool; and
   d. contacting the population of T cells with a DNA-PK inhibitor;
      thereby producing a population of edited T cells.
Embodiment_F 6. The method of embodiment_F 5, wherein the edited T cells comprise multiple genome edits per cell.
Embodiment_F 7. The method of any of the preceding embodiments_F, wherein the LNPs are preincubated with ApoE prior contacting the population of T cells with the LNPs.
Embodiment_F 8. The method of any of the preceding embodiments_F, wherein the LNPs are preincubated with ApoE3 prior contacting the population of T cells with the LNPs.
Embodiment_F 9. The method of any of the preceding embodiments_F, wherein the LNPs are preincubated with ApoE4 prior contacting the population of T cells with the LNPs.
Embodiment_F 10. The method of any of the preceding embodiments_F, wherein the population of T cells are contacted with LNPs comprising 2.5-10 μg/mL total RNA cargo.
Embodiment_F 11. The method of any of the preceding embodiments_F, wherein the population of T cells is contacted with 2-10 LNPs, e.g., two lipid nanoparticles (LNPs).
Embodiment_F 12. The method of any of the preceding embodiments_F, further comprising contacting the population of B cells with a donor nucleic acid for insertion into a target sequence.
Embodiment_F 13. The method of any of the preceding embodiments_F, wherein the method produces a population of T cells comprising at least 50%, 60%, 70%, 80%, 90%, 95%, 96%, 97%, 98%, or 99% of cells comprising a genome edit.
Embodiment_F 14. The method of embodiment_F 13, wherein the genome edit comprises an indel or a base edit and the population of T cells comprises at least 50%, 60%, 70%, 80%, 90%, 95%, 96%, 97%, 98%, or 99% of cells comprising a genome edit.
Embodiment_F 15. The method embodiments_F 13 and 14, wherein the genome edit comprises an insertion of an exogenous nucleic acid sequence into a target sequence and the population of T cells comprises at least 50%, 60%, 70%, 80%, 90%, or 95% of cells comprising a genome edit.
Embodiment_F 16. The method of embodiments_F 13-15, wherein the population of cells comprises edited T cells comprising at least two genome edits, wherein at least 50%, 60%, 70%, 80%, or 85% of cells comprise both genome edits.
Embodiment_F 17. The method of any of the preceding embodiments_F, wherein the method produces a population of T cells comprising edited T cells comprising multiple genome edits per cell, wherein fewer than 1% of the cells have a target-to-target translocations.
Embodiment_F 18. The method of any of the preceding embodiments_F, wherein the method produces a population of T cells comprising edited T cells comprising multiple genome edits per cell, wherein fewer than 0.5% of the cells have a target-to-target translocations.
Embodiment_F 19. The method of any of the preceding embodiments_F, wherein the method produces a population of T cells comprising edited T cells comprising multiple genome edits per cell, wherein fewer than 0.2% of the cells have a target-to-target translocations.
Embodiment_F 20. The method of any of the preceding embodiments_F, wherein the method produces a population of T cells comprising edited T cells comprising multiple genome edits per cell, wherein fewer than 0.1% of the cells have a target-to-target translocations.
Embodiment_F 21. The method of any of the preceding embodiments_F, wherein the method produces a population of T cells comprising edited T cells comprising multiple genome edits per cell, wherein the edited cells have less than 2 times the background level of reciprocal translocations, complex translocations, or off-target translocations.
Embodiment_F 22. The method of any of the preceding embodiments_F, wherein the edited T cells comprise CD4+ T cells.
Embodiment_F 23. The method of any of the preceding embodiments_F, wherein the edited T cells comprise CD8+ T cells.
Embodiment_F 24. The method of any of the preceding embodiments_F, wherein the edited T cells comprise memory T cells.
Embodiment_F 25. The method of any of the preceding embodiments_F, wherein the LNP composition comprises a guide RNA and an mRNA encoding the RNA guided-DNA binding agent, such as a Cas9, optionally an S. pyogenes Cas9.
Embodiment_F 26. The method of any of the preceding embodiments_F, wherein the LNP composition comprises a guide RNA and an mRNA encoding the RNA guided-DNA binding agent, wherein the RNA guided-DNA binding agent is a nickase.
Embodiment_F 27. The method of any of the preceding embodiments_F, wherein the LNP composition comprises a guide RNA and an mRNA encoding the RNA guided-DNA binding agent, wherein the RNA guided-DNA binding agent is a cleavase.
Embodiment_F 28. The method of any preceding embodiments_F, wherein the method does not comprise a selection step, optionally a physical selection step or a biochemical selection step.
Embodiment_F 29. The method of any preceding embodiments_F, wherein the methods are performed ex vivo.
Embodiment_F 30. A composition comprising a population of T cells comprising edited T cells, wherein the population of T cells comprises at least 50%, 60%, 70%, 80%, 90%, 95%, 96%, 97%, 98%, or 99% of cells comprising a genome edit.
Embodiment_F 31. A composition comprising a population of T cells comprising edited T cells, wherein the population of T cells comprises at least 50%, 60%, 70%, 80%, 90%, 95%, 96%, 97%, 98%, or 99% of cells comprising a genome edit, wherein the genome edit comprises an indel or a base edit.
Embodiment_F 32. A composition comprising a population of T cells comprising edited T cells, wherein the population of T cells comprises at least 50%, 60%, 70%, 80%, 90%, or 95% of cells comprising a genome edit, wherein the genome edit comprises an insertion of an exogenous nucleic acid sequence into a target sequence.
Embodiment_F 33. A composition comprising a population of T cells comprising edited T cells, wherein the population of T cells comprises at least 50%, 60%, 70%, 80%, or 85% of cells comprising at least two genome edits.
Embodiment_F 34. The composition of any of embodiments_F30-33, wherein fewer than 1% of the cells have a target-to-target translocations.
Embodiment_F 35. The composition of any of the preceding embodiments_F30-33, wherein fewer than 0.5% of the cells have a target-to-target translocations.
Embodiment_F 36. The composition of any of the preceding embodiments_F30-33, wherein fewer than 0.2% of the cells have a target-to-target translocations.
Embodiment_F 37. The composition of any of the preceding embodiments_F30-33, wherein fewer than fewer than 0.1% of the cells have a target-to-target translocations.
Embodiment_F 38. The composition of any of the preceding embodiments_F30-37, wherein the cells have less than 2 times the background level of reciprocal translocations, complex translocations, or off-target translocations.
Embodiment_F 39. A composition comprising a population of T cells comprising edited T cells, wherein the edited T cells are obtained by or obtainable by the method of any of embodiments_F1-29.

### VIII. EXAMPLES

### Example 1. General Methods

### Example 1.1. Preparation of Lipid Nucleic Acid Assemblies

In general, the lipid components were dissolved in 100% ethanol at various molar ratios. The RNA cargos (e.g., Cas9 mRNA and sgRNA) were dissolved in 25 mM citrate, 100 mM NaCl, pH 5.0, resulting in a concentration of RNA cargo of approximately 0.45 mg/mL.

Unless otherwise specified, the lipid nucleic acid assemblies contained ionizable Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG in a 50:38:9:3 molar ratio, respectively. The lipid nucleic acid assemblies were formulated with a lipid amine to RNA phosphate (N:P) molar ratio of about 6, and a ratio of gRNA to mRNA of 1:1 by weight, unless otherwise specified. In Examples 15-34, a ratio of gRNA to mRNA of 1:2 by weight was used, unless otherwise specified.

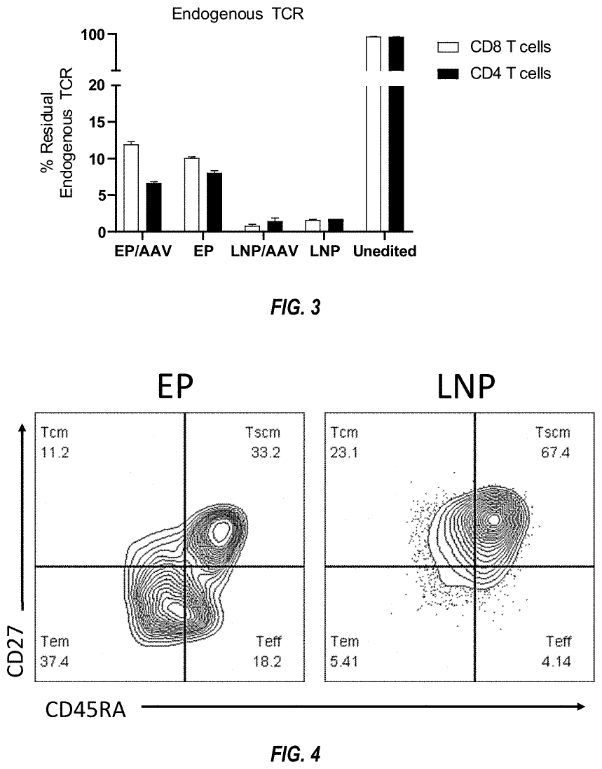

LNPs were prepared using a cross-flow technique utilizing impinging jet mixing of the lipid in ethanol with two volumes of RNA solutions and one volume of water. The lipids in ethanol were mixed through a mixing cross with the two volumes of RNA solution. A fourth stream of water was mixed with the outlet stream of the cross through an inline tee (See WO2016010840 FIG. 2). The LNPs were held for 1 hour at room temperature, and further diluted with water (approximately 1:1 v/v). LNPs were concentrated using tangential flow filtration, e.g. on a flat sheet cartridge (Sartorius, 100 kD MWCO) and buffer exchanged using PD-10 desalting columns (GE) into 50 mM Tris, 45 mM NaCl, 5% (w/v) sucrose, pH 7.5 (TSS). Alternatively, the LNP's were optionally concentrated using 100 kDa Amicon spin filter and buffer exchanged using PD-10 desalting columns (GE) into TSS. The resulting mixture was then filtered using a 0.2 μm sterile filter. The final LNP was stored at 4° C. or −80° C. until further use.

### Example 1.2. In Vitro Transcription (“IVT”) of Nuclease mRNA

Capped and polyadenylated mRNA containing N1-methyl pseudo-U was generated by in vitro transcription using a linearized plasmid DNA template and T7 RNA polymerase. Plasmid DNA containing a T7 promoter, a sequence for transcription, and a polyadenylation region was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts. The IVT reaction to generate modified mRNA was performed by incubating at 37° C. for 1.5-4 hours in the following conditions: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. TURBO DNase (ThermoFisher) was added to a final concentration of 0.01 U/μL, and the reaction was incubated for an additional 30 minutes to remove the DNA template. The mRNA was purified using a MegaClear Transcription Clean-up kit (ThermoFisher) or an RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA is purified using LiCl precipitation, ammonium acetate precipitation and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Streptococcus pyogenes (“Spy”) Cas9 mRNA was generated from plasmid DNA encoding an open reading frame according to SEQ ID Nos: 1-3 (see sequences in Table 89). BC22n mRNA was generated from plasmid DNA encoding an open reading frame according to SEQ ID Nos: 18. UGI mRNA was generated from plasmid DNA encoding an open reading frame according to SEQ ID Nos: 21. When the sequences cited in this paragraph are referred to below with respect to RNAs, it is understood that Ts should be replaced with Us (which were N1-methyl pseudouridines as described above). Messenger RNAs used in the Examples include a 5′ cap and a 3′ polyadenylation sequence e.g., up to 100 nts and are identified by in Table 89. Guide RNAs were chemically synthesized by methods known in the art.

### Example 1.3. Next-Generation Sequencing (“NGS”) and Analysis for On-Target Cleavage Efficiency

Genomic DNA was extracted using QuickExtract™ DNA Extraction Solution (Lucigen, Cat. QE09050) according to manufacturer's protocol.

To quantitatively determine the efficiency of editing at the target location in the genome, deep sequencing was utilized to identify the presence of insertions and deletions introduced by gene editing. PCR primers were designed around the target site within the gene of interest (e.g., TRAC) and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the human reference genome (e.g., hg38) after eliminating those having low quality scores. Reads that overlapped the target region of interest were re-aligned to the local genome sequence to improve the alignment. Then the number of wild type reads versus the number of reads which contain C-to-T mutations, C-to-A/G mutations or indels was calculated. Insertions and deletions were scored in a 20 bp region centered on the predicted Cas9 cleavage site. Indel percentage is defined as the total number of sequencing reads with one or more base inserted or deleted within the 20 bp scoring region divided by the total number of sequencing reads, including wild type. C-to-T mutations or C-to-A/G mutations were scored in a 40 bp region including 10 bp upstream and 10 bp downstream of the 20 bp sgRNA target sequence. The C-to-T editing percentage is defined as the total number of sequencing reads with either one or more C-to-T mutations within the 40 bp region divided by the total number of sequencing reads, including wild type. The percentage of C-to-A/G mutations are calculated similarly.

### Example 1.4. T Cell Culture Media Preparation

T cell culture media compositions used below are described here and in Table 2. “X-VIVO Base Media” consists of X-VIVO™ 15 Media, 1% Penstrep, 50 μM Beta-Mercaptoethanol, 10 mM NAC. “RPMI Base Media” consists of RPMI Media, 1% Penstrep, 2 mM L-Glutamine, 100 μM Non-essential amino acids, 1 mM Sodium Pyruvate, 10 mM HEPES Buffer, and 55 μM Beta-Mercaptoethanol. “CTS OpTmizer Base Media” consists of CTS OpTmizer Media, entire contents of supplements provided with the media, 1× Glutamax and 10 mM HEPES. In addition to above mentioned components, few variable media components used here are; 1. Serum (Fetal Bovine Serum (FBS) or Human Serum AB, and 2. Cytokines (IL-2, IL-7, IL-15) also described in Table 1. Media components are described in Table 2 below.

TABLE 1
Media components

| Media components | | Concentration | | Vendor | Catalog # |
|---|---|---|---|---|---|
| Base | RPMI Media | | | Corning | |
| Base | L-glutamine | 2 | mM | Corning | |
| optional | Pen-Strep | 1% | | Corning | 30-002-CI |
| Base | Non-essential | 100 | μM | Gibco | |
| | amino acids | | | | |
| Base | Sodium pyruvate | 1 | mM | Gibco | |
| Base | HEPES buffer | 10 | mM | Gibco | |
| Base | Beta- | 55 | μM | Fisher | ICN19470583 |
| | mercaptoethanol | | | | |
| Variable | FBS (Fetal | 10% | | Gibco | |
| | Bovine Serum) | | | | |
| Base | X-VIVO ™ | | | LONZA | BE02-060Q |
| | 15 Media | | | | |
| Base | Human Serum | 5% | | Gemini Bio | 100-512 |
| | AB | | | Products | |
| optional | Pen-Strep | 1% | | Corning | 30-002-CI |
| Base | Beta- | 50 | μM | Fisher | ICN19470583 |
| | mercaptoethanol | | | | |
| Base | N-Acetyl L- | 10 | mM | Fisher | ICN19460325 |
| | Cysteine (NAC) | | | | |
| Variable | rh IL-15 | 200 | Units/mL | Peprotech | 200-15 |
| Variable | rh-IL7 | 5 | ng/mL | Peprotech | 200-07 |
| Variable | rh-IL-2 | 5 | ng/mL | Peprotech | 200-02 |
| Base | CTS Optimizer | | | Thermo- | |
| | Media | | | Fisher | |
| Base | Supplement | All of the | | Thermo- | |
| | | contents | | Fisher | |
| | | supplied | | | |
| Variable | Human Serum | 5% | | Gemini Bio | 100-512 |
| | AB | | | Products | |
| Base | Glutamax | 1X | | | |
| Base | HEPES buffer | 10 | mM | Gibco | |
| Variable | rh IL-15 | 5 | ng/mL | Peprotech | 200-15 |
| Variable | rh-IL7 | 5 | ng/mL | Peprotech | 200-07 |
| Variable | rh-IL-2 | 200 | Units/mL | Peprotech | 200-02 |

T cells were thawed or cultured in T cell culture media as described by media numbers in Table 2 below, unless otherwise mentioned.

TABLE 2
T cell media compositions

| Media | | |
|---|---|---|
| Number | Media Composition | |
| 1 | X-VIVO Base media + IL-2 + IL-15 + IL7 | With Human |
| 2 | X-VIVO Base media + IL-2 + IL-15 | Serum AB |
| 3 | X-VIVO Base media + IL-2 + IL7 | |
| 4 | X-VIVO Base media + IL-2 | |
| 5 | X-VIVO Base media | |
| 6 | X-VIVO Base media | No Serum |
| 7 | X-VIVO Base media + IL-2 + IL-15 + IL7 | |
| 8 | X-VIVO Base media + IL-2 + IL-15 | |
| 9 | X-VIVO Base media + IL-2 + IL7 | |
| 10 | X-VIVO Base media + IL-2 | |
| 11 | RPMI Base media + IL-2 | With Fetal |
| 12 | RPMI Base media + IL-2 + IL-15 | Bovine |
| 13 | RPMI Base media + IL-2 + IL7 | Serum (FBS) |
| 14 | RPMI Base media + IL-2 + IL-15 + IL7 | |
| 15 | RPMI Base Media | |
| 16 | CTS Optimizer Base media + IL-2, IL7, | No Serum. |
| | IL-15 | |
| 17 | CTS Optimizer Base media with Serum + | With Human |
| | IL-2, IL7, IL-15 | Serum AB |

### Example 2. In Vitro Functional Characterization of T Cells Engineered with LNPs and Electroporation

To determine if the method of T cell engineering impacts the properties of resulting cells, we compared the in vitro characteristics of T cells genetically engineered via electroporation (EP) or lipid nanoparticles (LNP).

### Example 2.1. T Cell Preparation

Healthy human donor apheresis was obtained commercially (Hemacare), and cells were washed and re-suspended in CliniMACS PBS/EDTA buffer (Miltenyi cat. 130-070-525) on the LOVO device. T cells were isolated via positive selection using CD4 and CD8 magnetic beads (Miltenyi BioTec cat. 130-030-401/130-030-801) using the CliniMACS Plus and CliniMACS LS disposable kit. T cells were aliquoted into vials and cryopreserved in a 1:1 formulation of Cryostor CS10 (StemCell Technologies cat. 07930) and Plasmalyte A (Baxter cat. 2B2522X) for future use. Upon thaw, T cells were rested overnight at a density of 1. 5×10e6 cells/mL in Media Number 1, as described in Table 2. After overnight rest T cells were activated with TransAct (1:100 dilution, Miltenyi) for 48 hours prior to editing.

### Example 2.2. LNP Treatment of T Cells

LNPs containing Cas9 mRNA and a sgRNA targeting TRAC (G013006) (SEQ ID NO: 708) or TRBC (G016239) (SEQ ID NO: 707) in a ratio of gRNA to mRNA of 1:2 by weight were separately incubated in in Media Number 1, as described in Table 2 supplemented with 6% cynomolgus monkey serum (BioreclamationlVT, Cat. CYN220760) for 5 minutes at 37° C. Forty eight hours post activation, T cells were washed and suspended in in Media Number 1, as described in Table 2. Pre-incubated LNP mix was added to the each well to yield a final concentration of 1 μg/mL per LNP and 1×10e6 cells/mL T cells. AAV6 was used to deliver homology directed repair template (HDRT) encoding a WT1 targeting transgenic T cell receptor (tgTCR) flanked by homology arms for site-specific integration into the TRAC locus. AAV was added at a multiplicity of infection (MOI) of 3×10e5 genome copy units (GCU)/cell. Control groups including unedited T cells (no LNP or AAV) and T cells transfected with LNPs but not transduced with AAV were also included. After 24 hours, T cells were collected, centrifuged, and transferred to G-REX® plates (Wilson Wolf) in Media Number 1, as described in Table 2. T cells were cultured for 7 days, with media exchanges every other day, before being evaluated for expansion, tgTCR insertion and endogenous TCR knockout by flow cytometry. All groups were done with replicate wells (n=2). Expanded T cells were cryopreserved for functional assays as described below.

### Example 2.3. RNP Electroporation of T Cells

RNPs were formed at a 20 μM stock concentration by mixing Cas9 protein with heat denatured sgRNAs targeting TRAC (G013006) (SEQ ID NO: 708) or TRBC (G016239) (SEQ ID NO: 707) at a 2:1 guide:Cas9 ratio for 15 minutes. RNP stocks were stored at −80° C. until used. Forty-eight hours post activation, T cells were harvested, centrifuged, and resuspended at a concentration of 10-20×10e6 T cells/100 μL in P3 electroporation buffer (Lonza). The cell suspension was mixed with RNPs to achieve a final RNP concentration of 2 before being transferred to a Nucleofector Cuvette and electroporated using the manufacturer's pulse code. Electroporated T cells were immediately rested in 400 μL in Media Number 5, as described in Table 2 without cytokines for 10 minutes before being plated at a density of 1×10e6 cells/well/1 mL in Media Number 1, as described in Table 2 with AAV encoding a WT1 TCR at a MOI of 3×10e5 GCU/cell. After 24 hours T cells were harvested, washed, and added to G-REX® plates (Wilson Wolf) in Media Number 1, as described in Table 2. T cells were cultured for 7 days, with media exchanges every other day, before being evaluated for expansion, tgTCR insertion and endogenous TCR knockout by flow cytometry. Electroporation treated T cells were subsequently cultured for 4 additional days prior to being cryopreserved before being analyzed by flow cytometry again and evaluated in T cell functional assays.

### Example 2.4.1 T Cell Expansion

Cells were counted using Vi-CELL cell counter (Beckman Coulter) and fold expansion was calculated by dividing cell yield by the starting cell count at the time of insertion. Cells treated with LNPs showed levels of T cell expansion post-editing comparable to unedited T cells and a more than 2-fold greater expansion than cells treated with electroporation as shown in Table 3 and FIG. 1. The more rapid expansion of LNP treated cells compared to electroporated cells permitted a shorter manufacturing time (10 vs 14 days) to yield a level of expansion that is desirable for clinical manufacturing (>50-fold increase post edit).

TABLE 3
Fold expansion after 10 or 14 days of total culture

| | | Fold Expansion | Fold Expansion |
|---|---|---|---|
| Group | Day | (mean) | (SD) |
| EP + AAV | 10 | 13 | 1 |
| EP | 10 | 20 | 1 |
| LNP +AAV | 10 | 84.5 | 2.5 |
| LNP | 10 | 88.5 | 1.5 |
| Unedited | 10 | 94 | 10 |
| EP + AAV | 14 | 37 | 1.4 |

### Example 2.4.2. Flow Cytometry

On day 7 post-edit T cells were phenotyped by flow cytometry to determine endogenous TCR knockout and tgTCR insertion rates as well as memory and exhaustion status. Briefly, T cells were incubated in cocktails of antibodies targeting CD3, CD4, CD8, Vb8, CD62L, CD45RO. Cells were subsequently washed, processed on a Cytoflex instrument (Beckman Coulter) and analyzed using the FlowJo software package. T cells were gated on size, CD4/CD8 status, and WT1 tgTCR expression (Vb8+CD3+). Vb8 identifies expression of the WT1 tgTCR.

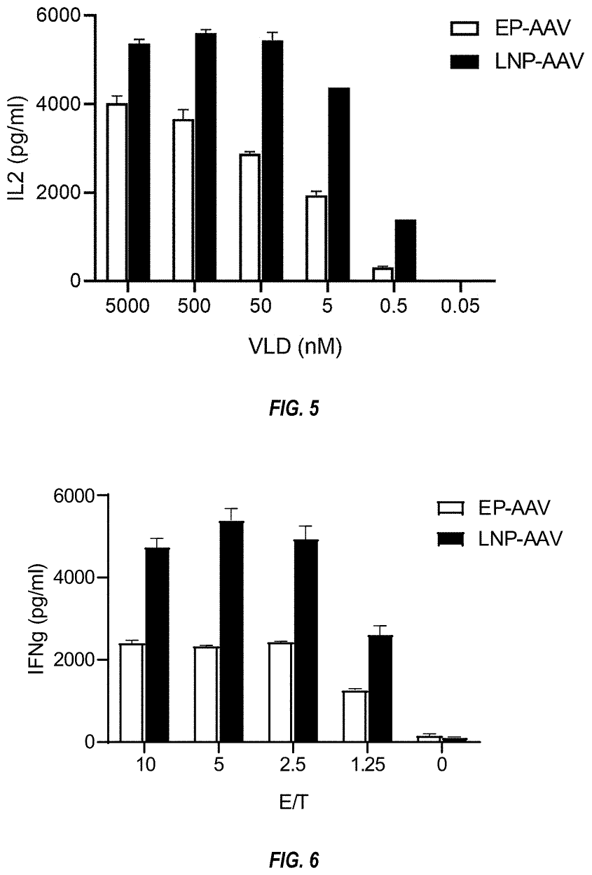

Endogenous TCR gene disruption and WT1 tgTCR insertion rates were assessed by flow cytometry. Table 4 and FIG. 2 show percent of CD3+Vb8+ TCR T cells. Table 5 and FIG. 3 show residual endogenous TCR (CD3+Vb8−).

TABLE 4
Transgenic TCR insertion rates in engineered T cells

| | | Insertion | |
|---|---|---|---|
| | | % | Insertion |
| Cell Type | Group | (mean) | (SD) |
| CD8 | EP + AAV | 68.5 | 0.4 |
| | EP only | 1.2 | 0.4 |
| | LNP + AAV | 60.9 | 1.4 |
| | LNP only | 0.8 | 0.0 |
| | Unedited | 5.3 | 0.1 |
| CD4 | EP + AAV | 71.55 | 0.85 |
| | EP only | 1.33 | 0.02 |
| | LNP + AAV | 55.4 | 1.3 |
| | LNP only | 0.855 | 0.115 |
| | Unedited | 6.295 | 0.395 |

TABLE 5
Residual endogenous TCR in engineered T cells

| | | Endogenous | Endogenous |
|---|---|---|---|
| | | TCR | TCR |
| Cell Type | Group | % (mean) | % (SD) |
| CD8 | EP + AAV | 12.0 | 0.3 |
| | EP only | 10.1 | 0.1 |
| | LNP + AAV | 0.8 | 0.1 |
| | LNP only | 1.6 | 0.0 |
| | Unedited | 94.5 | 0.1 |
| CD4 | EP + AAV | 6.67 | 0.11 |
| | EP only | 8.045 | 0.215 |
| | LNP + AAV | 1.46 | 0.29 |
| | LNP only | 1.765 | 0.005 |
| | Unedited | 93.55 | 0.35 |

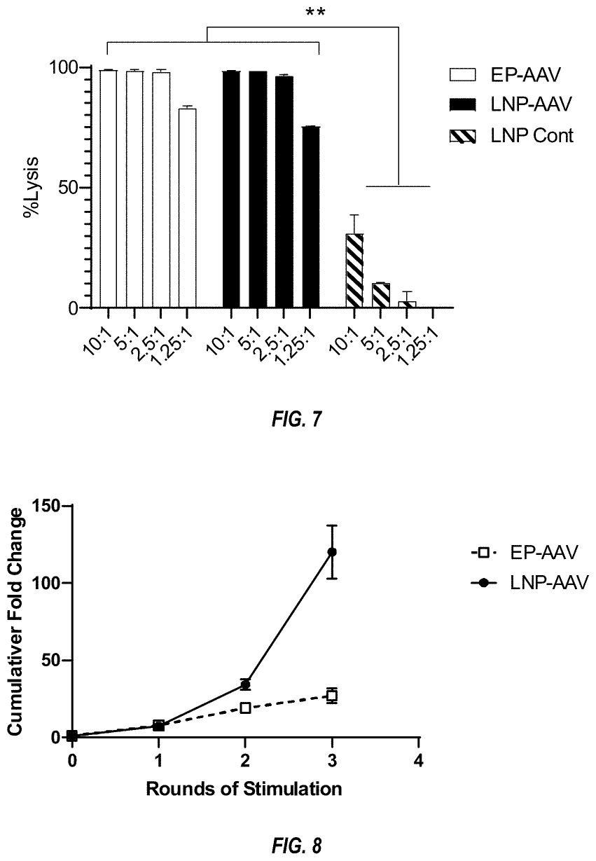

For a phenotypic analysis after expansion (Day 7 post edit for LNP group and Day 11 post edit for EP group) cryopreserved T cells were thawed, rested overnight in Media Number 1, as described in Table 2, and subsequently stained with CD3, CD4, CD8, CD45RA, IL-7R, CD45RO, CD95, LAG3, CD27, CD62L, TIM3, PD1, LAG3, for 20 mins in U-bottom 96-well plates. LNP engineered T cells harvested at day 10 vs RNP electroporated T cells harvested at day 14 show an increased CD45RA+CD27+ early stem-cell memory phenotype as shown in FIG. 4 and Table 6. The CD45RA+CD27+ early stem-cell memory phenotype which has been shown to correlate with increased persistence and therapeutic efficacy of cell therapy products was analyzed in the cell products.

TABLE 6
Memory phenotype of CD8+ T cells engineered
with LNP or electroporation

| CD8+ T cells | EP (Day 14) | LNP (Day 10) |
|---|---|---|
| % CD45RA+ CD27+ | 33.2 | 67.4 |
| % CD45RA− CD27+ | 11.2 | 23.1 |
| % CD54RA− CD27− | 37.4 | 18.2 |
| % CD45RA+ CD27− | 18.2 | 4.1 |

### Example 2.5. T Cell Functional Assays: Cytotoxicity and Cytokine Release

### Example 2.5.1. OCI-AML3 Co-Culture

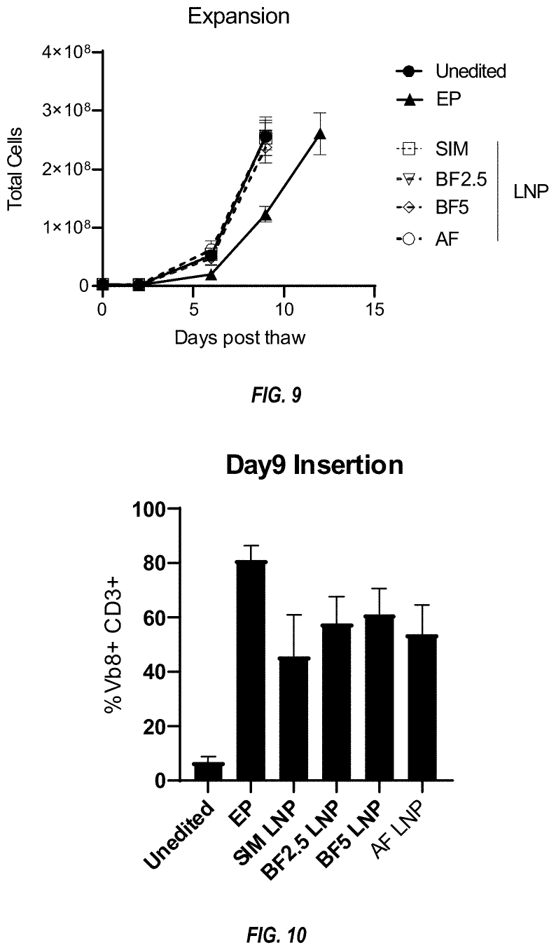

T cells engineered using the LNP and electroporation Cas9/sgRNA delivery processes were further evaluated for functional reactivity by measuring IL-2 secretion following co-culture with OCI-AML3 target cells pulsed with a titrated amount of WT1 peptide (VLDFAPPGA, hereafter referred to as the VLD peptide). OCI-AML3 cells were seeded at a density of 40,000 cells/well and incubated with titrated amounts of VLD peptide as shown in Table 7. Engineered T cells were added to pulsed OCI-AML3 cells at a 2.5:1 effector T cell:target cell (E:T) ratio in Media Number 5, as described in Table 2. After 24 hours of co-culture supernatants were harvested and IL-2 secretion quantified by ELISA according to manufacturer's protocol (R&D Duoset, Catalog #. DY202-5). In Table 7 and FIG. 5, LNP engineered T cells, relative to RNP engineered T cells, showed increased IL-2 production when co-cultured with VLD peptide pulsed OCI-AML3 cells.

TABLE 7
IL-2 secretion in co-cultures of LNP or RNP/EP engineered WT1 TCR T
cells with OCI-AML3 cells pulsed with titrated amounts of VLD peptide

| Group | VLD Peptide (nM) | IL-2 (mean pg/mL) | IL-2 (SD) |
|---|---|---|---|
| LNP + AAV | 5000 | 5369 | 65 |
| LNP + AAV | 500 | 5604 | 54 |
| LNP + AAV | 50 | 5436 | 130 |
| LNP + AAV | 5 | 4372 | nd |
| LNP + AAV | 0.5 | 1390 | nd |
| LNP + AAV | 0.05 | <LLOD | <LLOD |
| EP + AAV | 5000 | 4022 | 117 |
| EP + AAV | 500 | 3661 | 155 |
| EP + AAV | 50 | 2875 | 32 |
| EP + AAV | 5 | 1943 | 65 |
| EP + AAV | 0.5 | 308 | 16 |
| EP + AAV | 0.05 | <LLOD | <LLOD |

Not determined = nd; lower-limit of detection = LLOD

### Example 2.5.2. K562 Co-Culture

K562 cells transduced with HLA-A*02:01 and a luciferase reporter gene were treated with Mitomycin C (Tocris Biosciences, Catalog #3258) at 25 pg/mL for 1 hour to arrest cell division and subsequently co-cultured in duplicate with WT1 tgTCR T cells or TCR-null (LNP only) control T cells. After 24 hours, cytokine release (IFNγ) was quantified by ELISA (R&D Systems Cat. #DY285). After 48 hours T cell mediated cytotoxicity of target cells was quantified using Bright-GLO reagent according to manufacturer's protocol (Promega, E2610). Percent specific lysis was determined by the formula:

% Specific lysis=100−((experimental wells/target only control wells)×100)

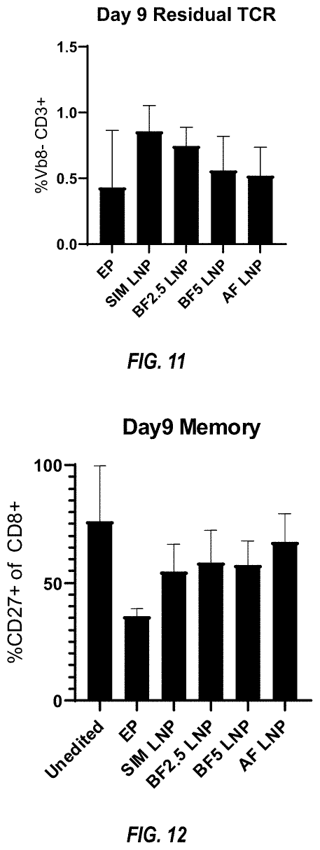

Table 8 and FIG. 6 shows interferon-gamma (IFNγ) release by engineered T cells in response to co-culture with K562 HLA-A*02:01 positive cells.

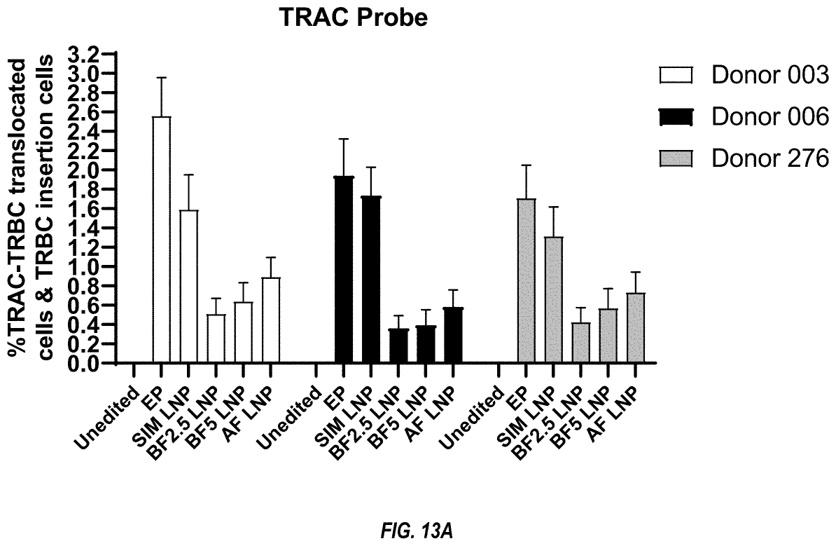

Table 9 and FIG. 7 shows specific lysis of K562 HLA-A*02:01 positive cells when co-cultured with engineered T cells.

TABLE 8
IFNγ release by engineered T cells co-cultured with K562 HLA-
A*02:01 positive cells

| Group | E:T | IFNγ (mean pg/mL) | IFNγ (SD) |
|---|---|---|---|
| LNP + AAV | 10 | 4742 | 151 |
| LNP + AAV | 5 | 5398 | 209 |
| LNP + AAV | 2.5 | 4937 | 227 |
| LNP + AAV | 1.25 | 2610 | 150 |
| LNP + AAV | T cell only | 106 | 9 |
| EP + AAV | 10 | 2405 | 46 |
| EP + AAV | 5 | 2324 | 25 |
| EP + AAV | 2.5 | 2420 | 30 |
| EP + AAV | 1.25 | 1263 | 19 |
| EP + AAV | T cell only | 150 | 35 |

TABLE 9
Specific lysis of K562 HLA-A*02:01 positive cells

| Group | E:T | % Specific Lysis | SD |
|---|---|---|---|
| LNP + AAV | 10 | 98.51 | 0.12 |
| LNP + AAV | 5 | 98.28 | 0.02 |
| LNP + AAV | 2.5 | 96.38 | 0.53 |
| LNP + AAV | 1.25 | 75.27 | 0.06 |
| EP + AAV | 10 | 98.92 | 0.14 |
| EP + AAV | 5 | 98.48 | 0.27 |
| EP + AAV | 2.5 | 98.20 | 0.50 |
| EP + AAV | 1.25 | 83.05 | 0.60 |
| LNP | 10 | 31.08 | 5.53 |
| LNP | 5 | 10.40 | 0.02 |
| LNP | 2.5 | 2.90 | 2.90 |
| LNP | 1.25 | 0.00 | 0.00 |

### Example 2.6. Targeted Cell-Mediated T Cell Re-Stimulation Assay

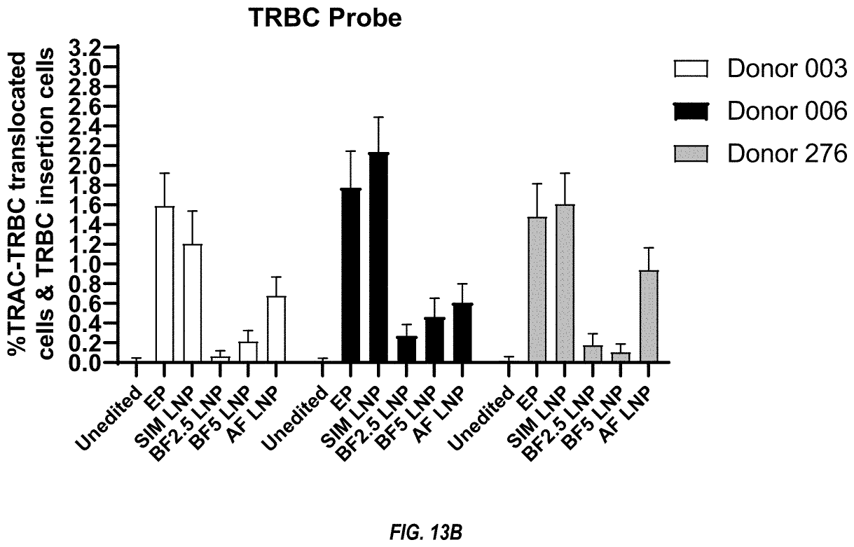

Briefly, effector T cells were co-cultured with OCI-AML3 target cells pulsed with 500 nM VLD peptide at 2.5:1 Effector T cell:target cell (E:T) ratio (Stimulation 1) in Media Number 5, as described in Table 2. After 5 days, effector T cell counts were recorded, and the cells were reseeded as for Stimulation 1. Five days after the second stimulation, cell counts were recorded, and samples were taken for flow cytometry analysis. The remaining cells were re-stimulated a third time as for Stimulation 1 with the exception of using a 5:1 E:T ratio. Five days after the third stimulation, cell counts were recorded, and samples were taken for flow cytometry analysis. Long term re-stimulation assays where LNP or RNP engineered T cells were co-cultured with VLD-peptide pulsed OCI-AML3 cells showed increased proliferation of LNP-engineered T cells over the course of multiple stimulations whereas RNP electroporated T cells have diminished proliferation after repeated stimulation (FIG. 8, Table 10).

TABLE 10
T cell expansion during three successive re-stimulations
with VLD peptide-pulsed OCI-AML3 cells
Data are shown as fold change in T cell number relative to
amount prior to stimulation 1.

| Group | Post Stimulation # | Cumulative Fold Change | SD |
|---|---|---|---|
| LNP + AAV | 1 | 7.2 | 0.4 |
| LNP + AAV | 2 | 34.2 | 3.5 |
| LNP + AAV | 3 | 120.1 | 17.2 |
| EP + AAV | 1 | 7.9 | 0.4 |
| EP + AAV | 2 | 19.0 | 1.0 |
| EP + AAV | 3 | 27.1 | 4.8 |

### Example 3. Structural Genomic Characterization of Electroporation and LNP Engineered T Cells

T cells were assayed for chromosomal translocations and in vitro functional characteristics following engineering by electroporation or LNP processes.

### Example 3.1. T Cell Engineering

T cells were isolated and cultured as in Example 2 with the exception that the T cell culture media was Media Number 17, as described in Table 2.

Electroporation treatment of T cells was performed as in Example 2, with the exception that T cells were electroporated at a density of 3-5×10e6 cells/100 uL in P3 buffer (Lonza X Kit L, Cat. V4X9-3012), and the entire contents of the electroporation cuvettes transferred to GREX plates (Wilson Wolf).

LNP treatment and activation of T cells was performed as in Example 2 with the following modifications. LNPs were generally prepared as described in Example 1 at a ratio of 50/9/39.5/1.5 Lipid A, cholesterol, DSPC, and PEG2k-DMG. LNPs contained either Cas9 mRNA and sgRNA G013006 (SEQ ID NO: 708) targeting TRAC or Cas9 mRNA and sgRNA G016239 (SEQ ID NO: 707) targeting TRBC. LNPs were prepared with a ratio of gRNA to mRNA of 1:2 by weight. LNPs were preincubated at a 2× concentration of 5 μg/mL (unless otherwise stated) in Media Number 17, as described in Table 2 supplemented with recombinant human ApoE3 (Peprotech, Catalog #350-02) at a concentration of 1 μg/mL for 15 minutes at 37° C. T cells were washed and suspended in in Media Number 16, as described in Table 2. Pre-incubated LNP was added to the each well to yield a final concentration of LNP as indicated in Table 11 with 0.5×10e6 cells/mL T cells. AAV6 was used to deliver homology directed repair template (HDRT) encoding a WT1 targeting tgTCR flanked by homology arms for site-specific integration into the TRAC locus. Post editing all T cells were expanded in a GREX plate.

Table 11 describes the editing steps for each sample. In some instances, T cells were edited in a sequential manner with LNPs as scheduled in Table 11. Briefly for the LNP Sequential 1 process (BF), T cells were treated with LNPs targeting TRBC as described above, with the exception of cells were kept at a density of 1×10e6 cells/mL and activated with a 1:100 dilution of TransAct as described in Example 2. LNPs were incubated with either 2.5% (BF2.5) or 5% (BF5) or 5% (AF) human AB serum (HABS). On Day 3 these edited T cells were treated with TRAC LNP and AAV as described above. For LNP AF, T cells were activated for 48 hours and treated with TRAC LNP and AAV as described above. The following day T cells were collected, washed, and treated with TRBC LNP for 24 hours before being transferred to a GREX plate. Simultaneous samples (LNP SIM) were edited on Day 3 with TRAC LNP, TRBC LNP and AAV.

TABLE 11
T cell engineering conditions

| Condition | Day 1 | Day 2 | Day 3 | Day 4 | Day 5 |
|---|---|---|---|---|---|
| Unedited | Thaw, Activate | | Wash | Expansion | |
| EP | Thaw, Activate | | RNP + AAV | Expansion | |
| LNP SIM LNP | Thaw, Activate | | TRAC LNP + | Expansion | |
| | | | TRBC LNP + | | |
| | | | AAV | | |
| BF2.5 LNP | Thaw, Activate, | | TRAC LNP + | Expansion | |
| | TRBC LNP 2.5% | | AAV | | |
| | HABS, 2.5 μg/ml | | | | |
| | LNP | | | | |
| BF5LNP | Thaw, Activate, | | TRAC LNP + | Expansion | |
| | TRBC LNP 5% | | AAV | | |
| | HABS, 5 μg/ml LNP | | | | |
| LNP AF | Thaw, Activate | | TRAC LNP + | TRBC LNP | Expansion |
| | | | AAV | | |

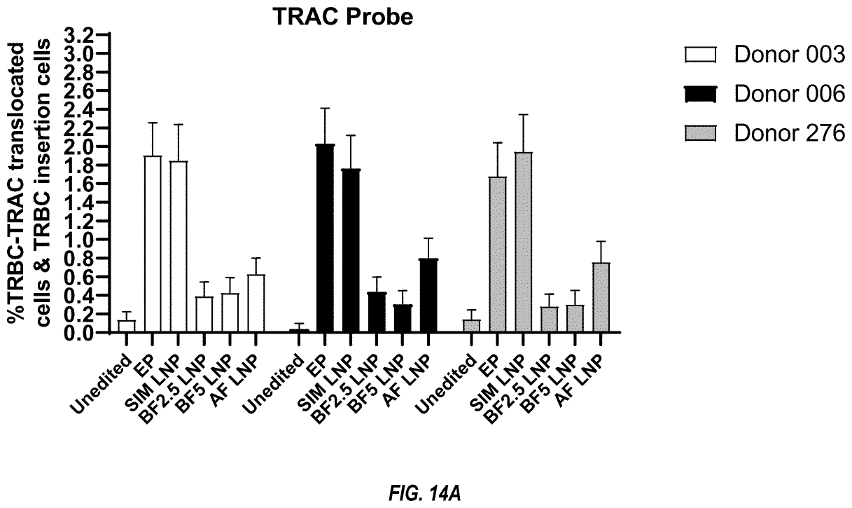

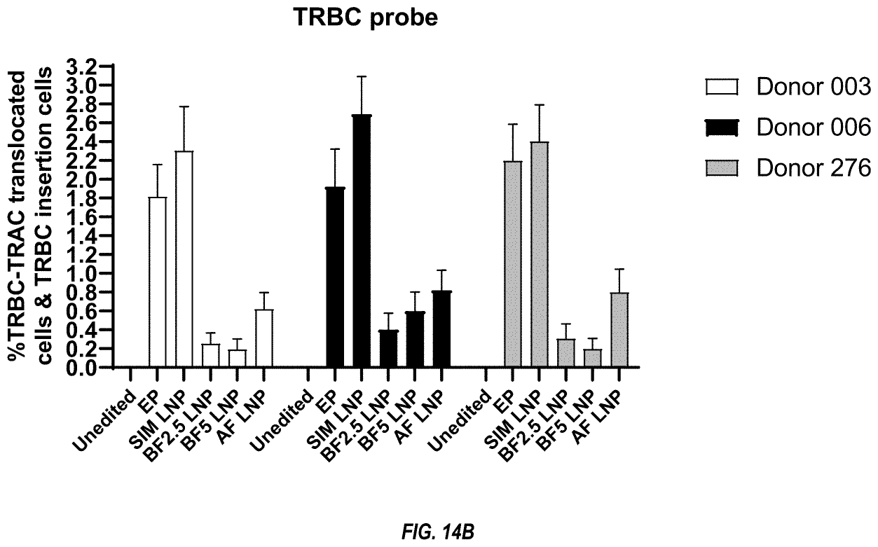

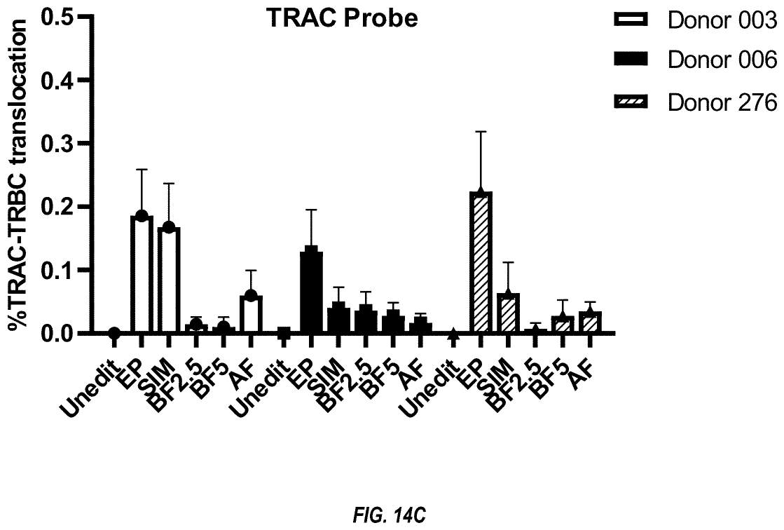

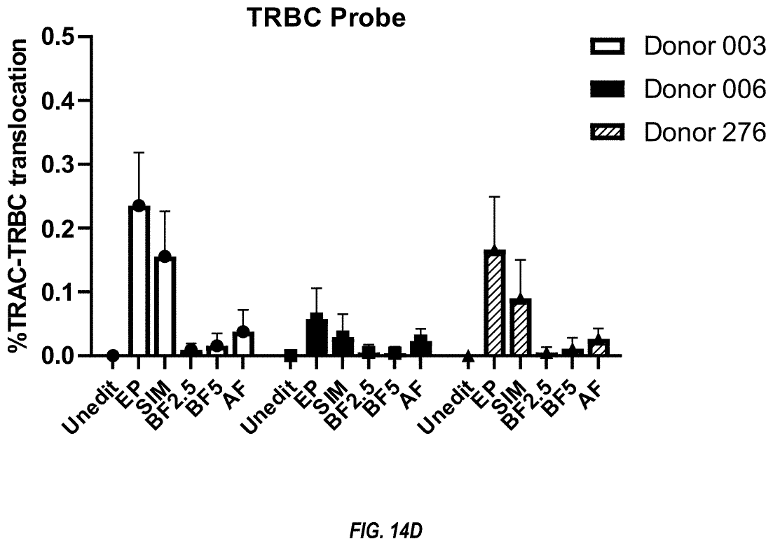

Following treatment and growth, T cells were harvested and assayed by flow cytometry as described in Example 2 using antibodies targeting CD3, Vb8, CD4, CD8, CD45RO, and CD27. T cells were preserved in Cryostore® CS10 media. Table 12 and FIG. 9 show expansion of T cell cultures post engineering. Fold expansion across the experiment is calculated by dividing the total cells at day 9 by the number of cells at day 0 (3 million) for each donor or for the group. In general, fold expansion can be calculated by dividing the total cell number by the number of cells seeded, e.g. counting nuclei by confocal microscopy. Table 13 and FIG. 10 show tgTCR insertion rates for engineered CD8+ T cells. Table 14 and FIG. 11 show the percentage of CD8+ T cells retaining endogenous TCR post treatment. Table 15 and FIG. 12 show the percentage of engineered T cells that are CD27+, a phenotype associated with memory cell phenotype.

TABLE 12
T cell expansion, total cells

| | | Mean | SD | | Repli- | Repli- | Repli- | Fold |
|---|---|---|---|---|---|---|---|---|
| Group | Day | (×10e6) | (×10e6) | N | cate | cate | cate | Change |
| Un- | 0 | 3 | 0 | 3 | 3.0 | 3.0 | 3.0 | |
| edited | 2 | 2.5 | 0.8 | 3 | 3.1 | 1.6 | 2.9 | |
| | 6 | 52 | 13.6 | 2 | 62.2 | 0.0 | 43.0 | |
| | 9 | 255 | 12.5 | 3 | 241.0 | 262.0 | 263.0 | 85 |
| EP | 0 | 3 | 0 | 3 | 3.0 | 3.0 | 3.0 | |
| | 2 | 3.0 | 5.4 | 3 | 3.0 | 2.5 | 3.5 | |
| | 6 | 20.4 | 7.1 | 3 | 28.0 | 19.3 | 13.9 | |
| | 9 | 123 | 22.8 | 3 | 149.0 | 109.0 | 110.0 | 41 |
| | 12 | 261 | 61.7 | 3 | 325.0 | 255.0 | 202.0 | 87 |
| SIM | 0 | 3 | 0 | 3 | 3.0 | 3.0 | 3.0 | |
| LNP | 2 | 2.8 | 1.0 | 3 | 3.9 | 1.9 | 2.8 | |
| | 6 | 54 | 15.6 | 3 | 71.7 | 51.1 | 41.1 | |
| | 9 | 253 | 51.5 | 3 | 312.0 | 230.0 | 217.0 | 84.3 |
| BF2.5 | 0 | 3 | 0 | 3 | 3.0 | 3.0 | 3.0 | |
| LNP | 2 | 2.42 | 0.3 | 3 | 2.7 | 2.1 | 2.4 | |
| | 6 | 51.1 | 23.9 | 3 | 78.4 | 41.4 | 33.5 | |
| | 9 | 256 | 57.0 | 3 | 317.0 | 247.0 | 204.0 | 85.3 |
| BF5 | 0 | 3 | 0 | 3 | 3.0 | 3.0 | 3.0 | |
| LNP | 2 | 1.94 | 0.5 | 3 | 2.2 | 1.4 | 2.3 | |
| | 6 | 48.1 | 20.9 | 3 | 71.3 | 42.5 | 30.6 | |
| | 9 | 238 | 47.9 | 3 | 293.0 | 210.0 | 210.0 | 79.3 |
| AF | 0 | 3 | 0 | 3 | 3.0 | 3.0 | 3.0 | |
| LNP | 2 | 2.52 | 0.5 | 3 | 3.1 | 2.0 | 2.5 | |
| | 6 | 62.5 | 25.8 | 3 | 92.3 | 48.1 | 47.1 | |
| | 9 | 256 | 40.9 | 3 | 293.0 | 263.0 | 212.0 | 85.3 |

TABLE 13
Transgenic TCR insertion rates into CD8+ T cells

| | Insertion | | | | | |
|---|---|---|---|---|---|---|
| | % | | | Repli- | Repli- | Repli- |
| Group | (mean) | SD | N | cate | cate | cate |
| Unedited | 6.7 | 2.0 | 3 | 4.9 | 6.5 | 8.8 |
| EP | 81.1 | 5.4 | 3 | 76.7 | 87.2 | 79.6 |
| SIM LNP | 45.7 | 15.2 | 3 | 30.3 | 60.7 | 46.2 |
| BF2.5 LNP | 57.7 | 9.8 | 3 | 46.8 | 66.1 | 60.2 |
| BF5 LNP | 61.2 | 9.5 | 3 | 51.8 | 70.8 | 61.2 |
| AF LNP | 53.9 | 10.7 | 3 | 41.6 | 61.7 | 58.4 |

TABLE 14
Residual endogenous TCR in CD8+ T cells

| | Endogenous | | | | | |
|---|---|---|---|---|---|---|
| | TCR % | | | | | |
| Group | (mean) | SD | N | Replicate | Replicate | Replicate |
| Unedited | 93.1 | 1.61 | 3 | 94.90 | 93.40 | 91.00 |
| EP | 0.43 | 0.43 | 3 | 0.93 | 0.22 | 0.14 |
| SIM LNP | 0.85 | 0.19 | 3 | 0.98 | 0.63 | 0.96 |
| BF2.5 LNP | 0.75 | 0.14 | 3 | 0.90 | 0.62 | 0.72 |
| BF5 LNP | 0.56 | 0.26 | 3 | 0.77 | 0.64 | 0.27 |
| AF LNP | 0.52 | 0.22 | 3 | 0.75 | 0.49 | 0.32 |

TABLE 15
Memory phenotype

| | % CD27 + | | | | | |
|---|---|---|---|---|---|---|
| Group | (mean) | SD | N | Replicate | Replicate | Replicate |
| Unedited | 76.1 | 23.6 | 3 | 91.4 | 88.0 | 48.9 |
| EP | 35.8 | 3.4 | 3 | 36.1 | 39.0 | 32.3 |
| SIM LNP | 54.9 | 11.6 | 3 | 64.4 | 58.4 | 42.0 |
| BF2.5 LNP | 58.7 | 13.5 | 3 | 68.6 | 64.3 | 43.3 |
| BF5 LNP | 57.5 | 10.4 | 3 | 66.7 | 59.6 | 46.2 |
| AF LNP | 67.4 | 11.8 | 3 | 76.4 | 71.7 | 54.0 |

### Example 3.2. Translocation Analysis and Insertion into TRBC Loci by Droplet Digital™ PCR

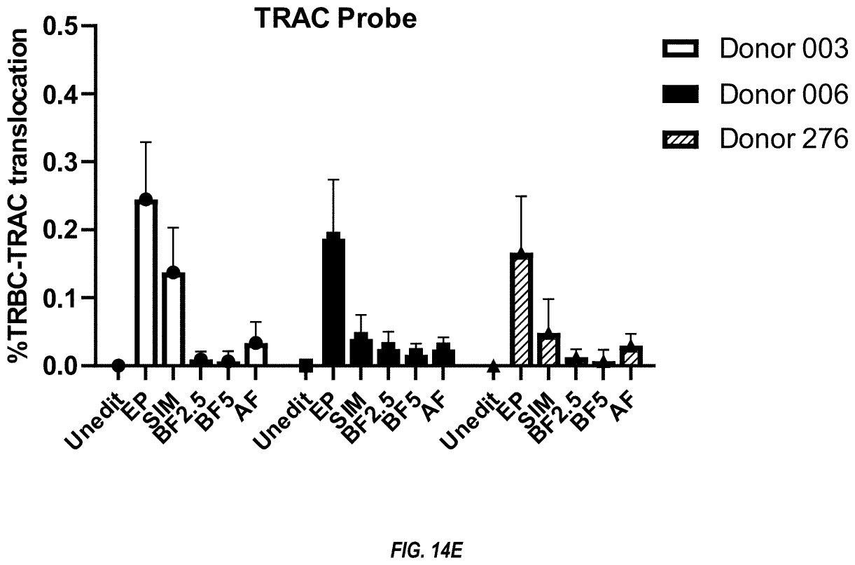

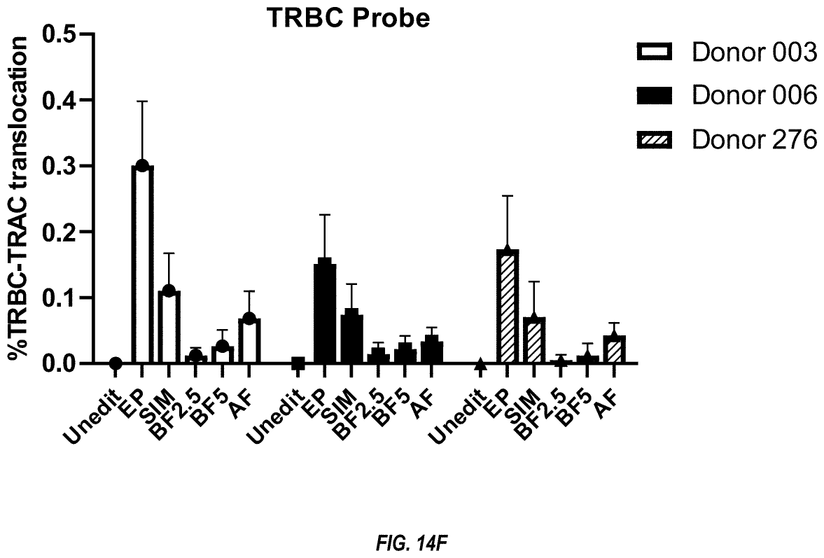

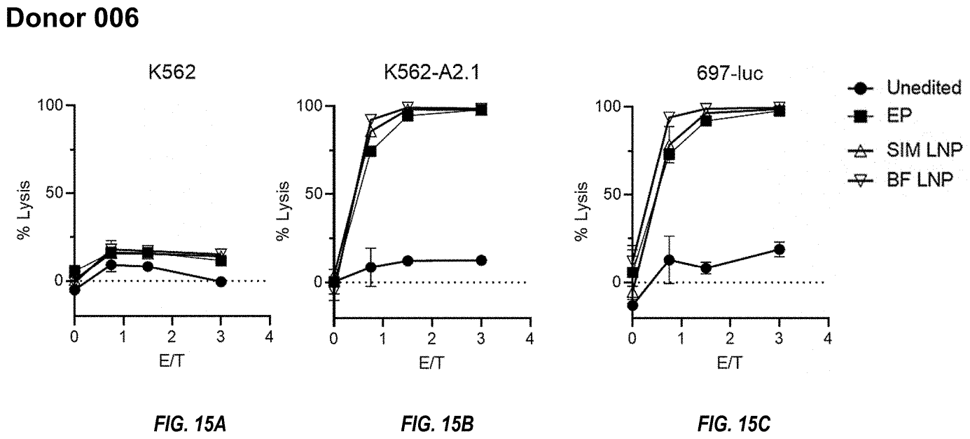

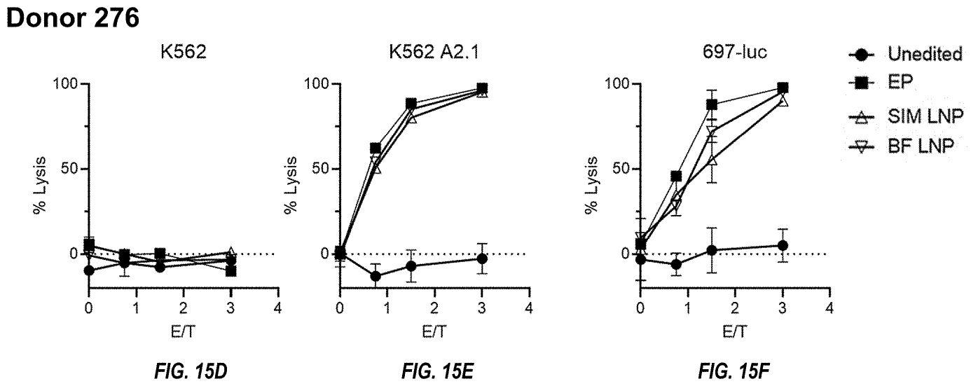

Translocations between the TRAC locus and the TRBC loci and insertion into TRBC loci was assayed using Droplet Digital™ PCR (ddPCR). Briefly, gDNA was isolated from T cell samples using DNeasy Blood and Tissue Kits (Qiagen, Cat. 69506) according to the manufacturer's protocols. ddPCR primers were selected to amplify TRAC-TRBC and TRBC-TRAC junctions, which detect TRAC-TRBC and TRBC-TRAC translocations as well as insertion of the selected TCR AAV construct into the TRBC loci by homology-independent, random integration. The ddPCR assay was carried out according to manufacturer's protocols. Briefly 100 ng of gDNA was prepared with 2× ddPCR Supermix for Probes (Biorad, Cat. 1863024) and Hind III HF (New England Biolabs, R3104S), validated primers at 900 nM and probes at 250 nM. The samples were processed with the QX200™ Droplet Generator (Biorad, Cat. 1864002), subjected to thermocycling. The cycle parameters were as follows: enzymatic activation for 10 min at 95° C.; 50 cycles of denaturation for 30 s at 94° C., annealing for 1 min at 60° C., and extension for 4 min at 72° C.; enzymatic deactivation for 10 min at 98° C., and hold at 4° C. Droplet fluorescence was measured using QX200TM Droplet Reader (Biorad, Cat. 1864003) and data analyzed with the QuantaSoft™ Software, Regulatory Edition (Biorad, Cat. 1864011). Percentage of TRAC-TRBC translocated and TRBC insertion cells (FIG. 13A (TRAC probe) and FIG. 13B (TRBC probe)) and TRBC-TRAC (FIG. 14A (TRAC probe) and FIG. 14B (TRBC probe)) translocated and TRBC insertion cells are shown in Table 16A).

TABLE 16A
Percent translocated and TRBC insertion cells

| | | Donor 003 | | | Donor 006 | | | Donor 276 | | |
|---|---|---|---|---|---|---|---|---|---|---|
| ddPCR | | | Pois- | Pois- | | Pois- | Pois- | | Pois- | Pois- |
| con- | | | son | son | | son | son | | son | son |
| ditions | Sample | Mean | Max | Min | Mean | Max | Min | Mean | Max | Min |
| TRAC- | Unedited | 0.00 | 0.00 | 0.00 | 0.00 | 0.00 | 0.00 | 0.00 | 0.00 | 0.00 |
| TRBC | EP | 2.56 | 2.96 | 2.16 | 1.94 | 2.32 | 1.56 | 1.71 | 2.05 | 1.35 |
| TRAC | SIM LNP | 1.59 | 1.95 | 1.22 | 1.73 | 2.03 | 1.43 | 1.31 | 1.62 | 1.00 |
| probe | BF2.5 | 0.51 | 0.67 | 0.34 | 0.36 | 0.49 | 0.23 | 0.42 | 0.57 | 0.27 |
| | LNP | | | | | | | | | |
| | BF5 LNP | 0.63 | 0.83 | 0.44 | 0.39 | 0.55 | 0.23 | 0.57 | 0.77 | 0.36 |
| | AF LNP | 0.89 | 1.09 | 0.68 | 0.58 | 0.76 | 0.41 | 0.73 | 0.94 | 0.51 |
| TRAC- | Unedited | 0.01 | 0.05 | 0.00 | 0.01 | 0.04 | 0.00 | 0.02 | 0.06 | 0.00 |
| TRBC | EP | 1.59 | 1.92 | 1.25 | 1.77 | 2.15 | 1.39 | 1.48 | 1.81 | 1.14 |
| TRBC | SIM LNP | 1.20 | 1.54 | 0.87 | 2.14 | 2.49 | 1.78 | 1.61 | 1.92 | 1.30 |
| probe | BF2.5 | 0.06 | 0.12 | 0.01 | 0.27 | 0.39 | 0.16 | 0.17 | 0.29 | 0.06 |
| | LNP | | | | | | | | | |
| | BF5 LNP | 0.21 | 0.32 | 0.10 | 0.46 | 0.65 | 0.28 | 0.10 | 0.19 | 0.02 |
| | AF LNP | 0.68 | 0.87 | 0.48 | 0.61 | 0.80 | 0.41 | 0.94 | 1.16 | 0.71 |
| TRBC- | Unedited | 0.00 | 0.00 | 0.00 | 0.00 | 0.00 | 0.00 | 0.00 | 0.00 | 0.00 |
| TRAC | EP | 1.81 | 2.16 | 1.47 | 1.92 | 2.32 | 1.53 | 2.20 | 2.59 | 1.81 |
| TRAC | SIM LNP | 2.30 | 2.77 | 1.83 | 2.69 | 3.09 | 2.29 | 2.40 | 2.79 | 2.01 |
| probe | BF2.5 | 0.25 | 0.37 | 0.13 | 0.40 | 0.58 | 0.23 | 0.31 | 0.46 | 0.15 |
| | LNP | | | | | | | | | |
| | BF5 LNP | 0.19 | 0.30 | 0.08 | 0.60 | 0.80 | 0.40 | 0.20 | 0.31 | 0.08 |
| | AF LNP | 0.62 | 0.80 | 0.45 | 0.82 | 1.03 | 0.60 | 0.80 | 1.04 | 0.56 |
| TRBC- | Unedited | 0.13 | 0.22 | 0.04 | 0.04 | 0.10 | 0.00 | 0.14 | 0.24 | 0.03 |
| TRAC | EP | 1.90 | 2.25 | 1.55 | 2.03 | 2.41 | 1.66 | 1.68 | 2.04 | 1.30 |
| TRBC | SIM LNP | 1.84 | 2.23 | 1.46 | 1.76 | 2.12 | 1.42 | 1.94 | 2.34 | 1.54 |
| probe | BF2.5 | 0.39 | 0.54 | 0.23 | 0.44 | 0.60 | 0.28 | 0.28 | 0.41 | 0.14 |
| | LNP | | | | | | | | | |
| | BF5 LNP | 0.43 | 0.59 | 0.26 | 0.30 | 0.45 | 0.16 | 0.30 | 0.45 | 0.15 |
| | AF LNP | 0.63 | 0.80 | 0.45 | 0.80 | 1.01 | 0.58 | 0.75 | 0.98 | 0.53 |

To specifically quantify the translocation rates between TRAC loci and TRBC loci and avoid the detection of homology-independent, random TRBC insertion, a set of new primers were designed to amplify the amplicon spanning the junction of TRAC-TRBC or TRBC-TRAC translocation site. The forward and reverse primers were either in the TRAC loci (outside of the AAV homology arm) or TRBC loci, respectively. Probes targeting either TRAC or TRBC loci were designed to recognize the amplified translocation amplicon. The new set of primer and probes allow the specific detection of translocations between TRAC loci and TRBC loci but will not detect the homology-independent, random integration in the TRBC loci as described above. The translocations between TRAC loci and TRBC loci were assayed using the new set of primers and probes. The ddPCR process was carried out as described above. Percentage of TRAC-TRBC translocated cells (FIG. 14C (TRAC probe) and FIG. 14D (TRBC probe)) and TRBC-TRAC (FIG. 14E (TRAC probe) and FIG. 14F (TRBC probe)) translocated cells are shown in Table 16B).

TABLE 16B
Percent translocated cells

| | | Donor 003 | | | Donor 006 | | | Donor 276 | | |
|---|---|---|---|---|---|---|---|---|---|---|
| ddPCR | | | Pois- | Pois- | | Pois- | Pois- | | Pois- | Pois- |
| con- | | | son | son | | son | son | | son | son |
| ditions | Sample | Mean | Max | Min | Mean | Max | Min | Mean | Max | Min |
| TRAC- | Unedited | 0.00 | 0.00 | 0.00 | 0.00 | 0.00 | 0.00 | 0.00 | 0.00 | 0.00 |
| TRBC | EP | 0.19 | 0.26 | 0.11 | 0.13 | 0.20 | 0.06 | 0.22 | 0.32 | 0.13 |
| TRAC | SIM LNP | 0.17 | 0.24 | 0.10 | 0.04 | 0.07 | 0.01 | 0.06 | 0.11 | 0.02 |
| probe | BF2.5 | 0.01 | 0.03 | 0.00 | 0.04 | 0.07 | 0.01 | 0.01 | 0.02 | 0.00 |
| | LNP | | | | | | | | | |
| | BF5 LNP | 0.01 | 0.03 | 0.00 | 0.03 | 0.05 | 0.01 | 0.03 | 0.05 | 0.00 |
| | AF LNP | 0.06 | 0.10 | 0.02 | 0.02 | 0.03 | 0.00 | 0.03 | 0.05 | 0.02 |
| TRAC- | Unedited | 0.00 | 0.00 | 0.00 | 0.00 | 0.00 | 0.00 | 0.00 | 0.00 | 0.00 |
| TRBC | EP | 0.24 | 0.32 | 0.15 | 0.06 | 0.11 | 0.01 | 0.17 | 0.25 | 0.08 |
| TRBC | SIM LNP | 0.16 | 0.23 | 0.08 | 0.03 | 0.07 | 0.00 | 0.09 | 0.15 | 0.03 |
| probe | BF2.5 | 0.01 | 0.02 | 0.00 | 0.01 | 0.02 | 0.00 | 0.01 | 0.01 | 0.00 |
| | LNP | | | | | | | | | |
| | BF5 LNP | 0.02 | 0.03 | 0.00 | 0.00 | 0.01 | 0.00 | 0.01 | 0.03 | 0.00 |
| | AF LNP | 0.04 | 0.07 | 0.00 | 0.02 | 0.04 | 0.00 | 0.03 | 0.04 | 0.01 |
| TRBC- | Unedited | 0.00 | 0.00 | 0.00 | 0.00 | 0.00 | 0.00 | 0.00 | 0.00 | 0.00 |
| TRAC | EP | 0.30 | 0.40 | 0.20 | 0.15 | 0.23 | 0.08 | 0.17 | 0.25 | 0.09 |
| TRBC | SIM LNP | 0.11 | 0.17 | 0.05 | 0.07 | 0.12 | 0.03 | 0.07 | 0.12 | 0.02 |
| probe | BF2.5 | 0.01 | 0.02 | 0.00 | 0.01 | 0.03 | 0.00 | 0.01 | 0.01 | 0.00 |
| | LNP | | | | | | | | | |
| | BF5 LNP | 0.03 | 0.05 | 0.00 | 0.02 | 0.04 | 0.00 | 0.01 | 0.03 | 0.00 |
| | AF LNP | 0.07 | 0.11 | 0.03 | 0.03 | 0.05 | 0.01 | 0.04 | 0.06 | 0.02 |
| TRBC- | Unedited | 0.00 | 0.00 | 0.00 | 0.00 | 0.00 | 0.00 | 0.00 | 0.00 | 0.00 |
| TRAC | EP | 0.24 | 0.33 | 0.16 | 0.19 | 0.27 | 0.10 | 0.17 | 0.25 | 0.08 |
| TRAC | SIM LNP | 0.14 | 0.20 | 0.07 | 0.04 | 0.07 | 0.00 | 0.05 | 0.10 | 0.00 |
| probe | BF2.5 | 0.01 | 0.02 | 0.00 | 0.02 | 0.05 | 0.00 | 0.01 | 0.02 | 0.00 |
| | LNP | | | | | | | | | |
| | BF5 LNP | 0.01 | 0.02 | 0.00 | 0.02 | 0.03 | 0.00 | 0.01 | 0.02 | 0.00 |
| | AF LNP | 0.03 | 0.06 | 0.00 | 0.02 | 0.04 | 0.01 | 0.03 | 0.05 | 0.01 |

### Example 3.3. Luciferase-Based Target Cell Killing Assay

T cells were further characterized for in vitro functional characteristics. B cell acute lymphoblastic leukemia cell line 697 (ACC 42) was obtained from the Deutsche Zammlung von Mikroorganismen and Zellkulteren GmbH (DSMZ) (Braunschweig Germany). Cells were transduced with LV-SFFV-Luc2-P2A-EmGFP lentiviral vector (Imanis Bioscience, Catalog #LV050-L) in the presence of polybrene (Millipore Sigma, Catalog #TR-1003) following the manufacturer's protocol. Clonal populations were screened for luciferase activity by measurement of bioluminescence intensity. 697-Luc2 cells were cultured at 37° C., 95% humidity, 5% CO2 in RPMI-1640 medium (Corning/Cellgro, Catalog #10-040-CM) supplemented with 10% fetal bovine serum (Gibco, Catalog #A38402-01), 5% Penicillin/Streptomycin (Gibco, Catalog #15140-122) and Glutamax (Gibco, Catalog #35050-061).

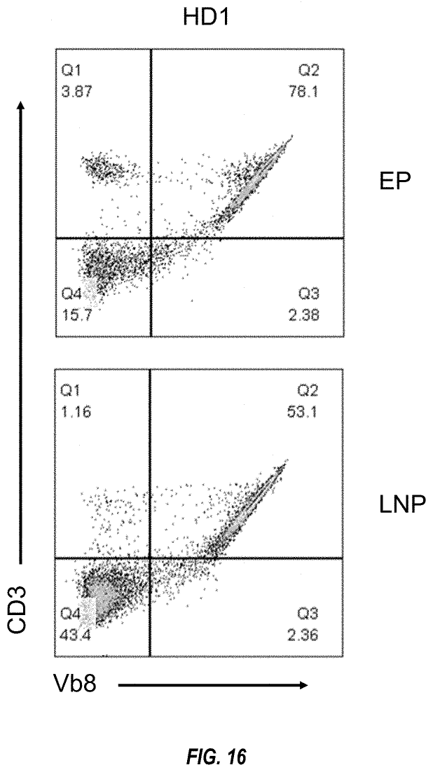

TCR-T cell mediated cytotoxicity of the WT1 expressing HLA-A02:01 targets (697-Luc2, K562 HLA-A*02:01-Luc2) and a negative control K562-Luc2 was assayed. For this the LNP edited WT1 TCR T cells or Unedited control T cells were co-cultured with the above target cell lines at effector-to-target ratios of 3:1, 1.5:1, and 0.75:1 for 48 hours. Luciferase signal was then detected using Bright-GLO reagent and analyzed as described in Example 2. Specific lysis is shown in Table 17 and FIG. 15A-F.

TABLE 17
Specific lysis of target cells by engineered T cells

| | | | Unedited | | | EP | | | SIM LNP | | | BF2.5 LNP | | |
|---|---|---|---|---|---|---|---|---|---|---|---|---|---|---|
| Donor | Target | E/T | Mean | SD | N | Mean | SD | N | Mean | SD | N | Mean | SD | N |
| 006 | K562 | 3 | −0.4 | 1.4 | 2 | 11.7 | 1.4 | 2 | 14.1 | 2.1 | 2 | 15.3 | 1.7 | 2 |
| | | 1.5 | 8.3 | 0.2 | 2 | 16.3 | 2.1 | 2 | 15.7 | 1.6 | 2 | 17.2 | 1.4 | 2 |
| | | 0.75 | 9.3 | 4.0 | 2 | 16.4 | 1.0 | 2 | 16.0 | 1.7 | 2 | 18.0 | 5.1 | 2 |
| | | 0 | −5.1 | 2.8 | 2 | 5.5 | 3.6 | 2 | 0.5 | 4.5 | 2 | −1.0 | 4.3 | 2 |
| | K562 | 3 | 12.7 | 1.5 | 2 | 97.9 | 0.0 | 2 | 97.8 | 0.0 | 2 | 98.6 | 0.4 | 2 |
| | HLA | 1.5 | 12.4 | 2.1 | 2 | 94.6 | 0.2 | 2 | 98.1 | 0.4 | 2 | 99.1 | 0.4 | 2 |
| | A*02:01 | 0.75 | 8.7 | 10.9 | 2 | 74.6 | 1.7 | 2 | 85.8 | 1.5 | 2 | 92.3 | 0.5 | 2 |
| | | 0 | 0.5 | 6.9 | 2 | 0.4 | 0.1 | 2 | 4.1 | 2.1 | 2 | −4.9 | 5.2 | 2 |
| | 697-luc | 3 | 19.0 | 4.2 | 2 | 97.8 | 1.3 | 2 | 98.8 | 0.7 | 2 | 99.5 | 0.1 | 2 |
| | | 1.5 | 8.3 | 3.4 | 2 | 92.0 | 0.2 | 2 | 96.4 | 3.8 | 2 | 98.9 | 0.4 | 2 |
| | | 0.75 | 13.0 | 13.5 | 2 | 73.0 | 3.2 | 2 | 78.4 | 10.3 | 2 | 93.9 | 0.3 | 2 |
| | | 0 | −12.8 | 2.1 | 2 | 5.9 | 15.3 | 2 | −5.2 | 3.1 | 2 | 12.2 | 6.5 | 2 |
| 276 | K562 | 3 | −3.8 | 3.5 | 2 | −9.8 | 1.6 | 2 | 1.2 | 1.5 | 2 | −3.2 | 2.5 | 2 |
| | | 1.5 | −7.7 | 2.6 | 2 | 0.4 | 0.5 | 2 | −4.7 | 3.5 | 2 | −3.5 | 1.0 | 2 |
| | | 0.75 | −5.2 | 7.7 | 2 | −0.4 | 1.0 | 2 | 0.2 | 1.9 | 2 | −5.3 | 1.8 | 2 |
| | | 0 | −9.6 | 11.7 | 2 | 5.4 | 4.7 | 2 | 4.8 | 4.0 | 2 | −0.7 | 8.8 | 2 |
| | K562 | 3 | −2.6 | 8.8 | 2 | 97.7 | 0.2 | 2 | 95.4 | 0.7 | 2 | 96.3 | 0.9 | 2 |
| | HLA | 1.5 | −7.0 | 9.3 | 2 | 88.7 | 0.4 | 2 | 80.2 | 2.7 | 2 | 85.3 | 1.2 | 2 |
| | A*02:01 | 0.75 | −12.9 | 7.1 | 2 | 62.2 | 0.3 | 2 | 50.6 | 2.3 | 2 | 54.1 | 1.7 | 2 |
| | | 0 | 0.4 | 3.1 | 2 | −0.1 | 4.0 | 2 | 1.0 | 3.4 | 2 | −1.4 | 6.1 | 2 |
| | 697-luc | 3 | 5.1 | 9.8 | 2 | 98.0 | 0.9 | 2 | 90.1 | 0.4 | 2 | 95.5 | 0.9 | 2 |
| | | 1.5 | 2.2 | 13.2 | 2 | 88.0 | 8.6 | 2 | 55.6 | 13.5 | 2 | 72.2 | 6.6 | 2 |
| | | 0.75 | −5.9 | 6.7 | 2 | 45.7 | 2.5 | 2 | 34.9 | 12.4 | 2 | 28.3 | 1.8 | 2 |
| | | 0 | −3.1 | 12.3 | 2 | 5.8 | 2.0 | 2 | 2.9 | 18.2 | 2 | 9.7 | 0.0 | 1 |

### Example 4. In Vivo Efficacy of LNP Engineered T Cells

LNP engineered T cells were assayed for in vivo efficacy to impact cancer cell growth and mortality in mice engrafted with B cell acute lymphoblastic leukemia cell line 697.

### Example 4.1. 697 Cell Preparation

Prior to engraftment, 697 cells as described in Example 3 were cultured at 37° C., 95% humidity, 5% CO2 in RPMI-1640 medium (Corning/Cellgro, Catalog #10-040-CM) supplemented with 10% fetal bovine serum (Gibco, Catalog #A38402-01), 5% Penicillin/Streptomycin (Gibco, Catalog #15140-122) and Glutamax (Gibco, Catalog #35050-061).

### Example 4.2. T Cell Engineering

T cells were isolated and prepared as in Example 2. LNPs were generally prepared as described in Example 1 at a ratio of 50/9/39.5/1.5 Lipid A, cholesterol, DSPC, and PEG2k-DMG. LNPs contained either mRNA encoding Cas9 (SEQ ID NO: 6) and sgRNA G013006 (SEQ ID NO: 708) targeting TRAC or Cas9 mRNA and sgRNA G016239 (SEQ ID NO: 707) targeting TRBC. LNP treatment of T cells was performed as in Example 2 with the following modifications. Forty-eight hours post activation, T cells were washed and suspended in Media Number 7, as described in Table 2. LNPs containing Cas9 mRNA and a sgRNA targeting TRAC or TRBC in a ratio of gRNA to mRNA of 1:2 by weight were incubated together (5 pg/mL each) in Media Number 1, as described in Table 2 supplemented to a final concentration of 1 pg/mL recombinant human ApoE3 (Peprotech, Catalog #350-02) for 15 minutes at 37° C. Pre-incubated LNP mix was added to the each well to yield a final concentration of 2.5 pg/mL per LNP and 0.5×10e6 cells/mL T cells. AAV6 was used to deliver homology directed repair template (HDRT) encoding either a WT1 targeting tgTCR (SEQ ID NO: 9) or GFP (Vigene; SEQ ID NO: 8), each flanked by homology arms for site-specific integration into the TRAC locus.

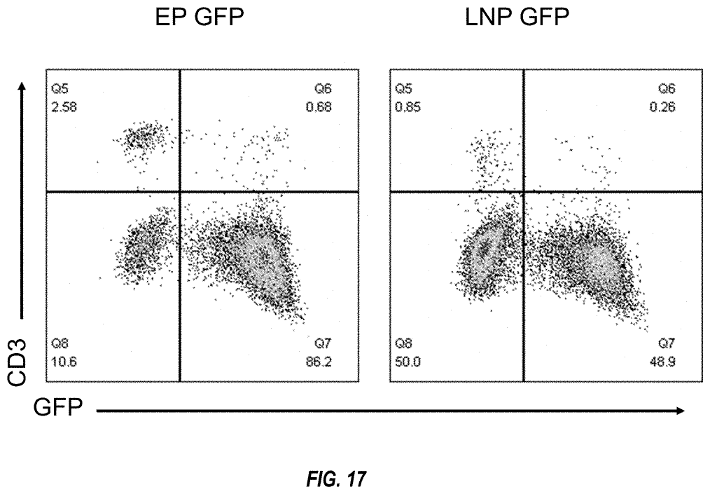

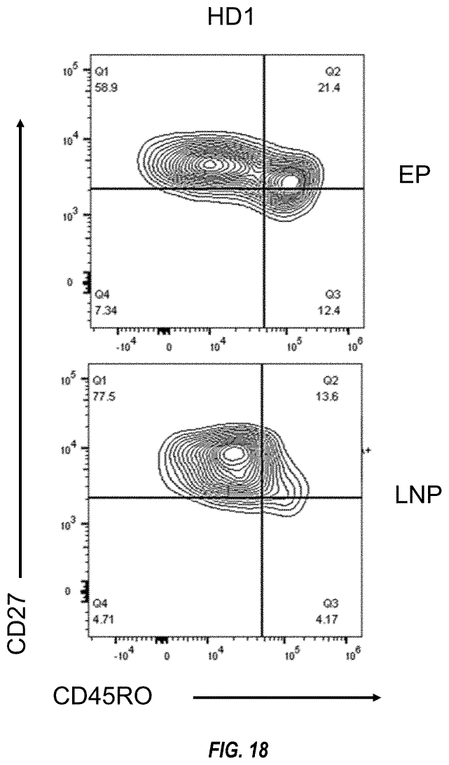

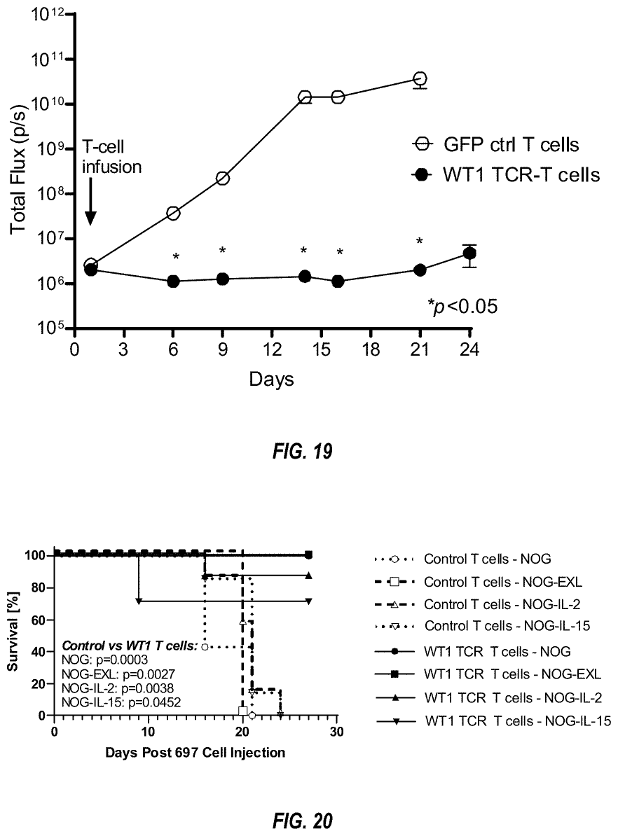

Following treatment and growth, T cells were harvested and assayed by flow cytometry as described in Example 2 using antibodies targeting CD3, CD4, CD8, CD45RO, and CD27. T cells were preserved in CryoStore® CS10 media. Table 18 and FIG. 16 show tgTCR insertion rates for engineered T cells. Table 19 and FIG. 17 show the percentage of CD8+ T cells retaining endogenous TCR post treatment. Table 20 and FIG. 18 show the percentage of engineered T cells that are CD45RO+CD27+, a phenotype associated with memory cell phenotype.

TABLE 18
Transgenic TCR insertion into CD8+ T cells

| T cells | EP | LNP |
|---|---|---|
| % CD3+ Vb8+ | 78.2 | 53.1 |
| % CD3− Vb8+ | 2.38 | 2.36 |
| % CD3− Vb8− | 15.7 | 43.4 |
| % CD3+ Vb8− | 3.87 | 1.16 |

TABLE 19
Retention of endogenous TCR

| T cells | EP | LNP |
|---|---|---|
| % CD3+ GFP+ | 0.68 | 0.26 |
| % CD3− GFP+ | 86.2 | 48.9 |
| % CD3− GFP− | 10.6 | 50.0 |
| % CD3+ GFP− | 2.58 | 0.85 |

TABLE 20
Memory phenotype CD8+ T cells

| CD8+ T cells | EP | LNP |
|---|---|---|
| % CD45RO+ CD27+ | 21.4 | 13.6 |
| % CD45RO− CD27+ | 58.9 | 77.5 |
| % CD54RO− CD27− | 7.34 | 4.71 |
| % CD45RO+ CD27− | 12.4 | 4.17 |

### Example 4.3. Engineered T Cell Efficacy In Vivo

Four humanized, immunodeficient mouse lines obtained from Taconic Biosciences were engrafted with 697 cells: NOG-h IL-2 (Model #: 13440-F), NOG-IL-15 (Model #: 13683-F), NOG (Model #: NOG-F) and NOG-EXL (Model 13395-F). Twenty-four hours after sub-lethal irradiation of 200 rad mice were inoculated intravenously with 0.2×10e6 697-Luc2 leukemia cells. Mice were warmed under a heating lamp for 3-5 minutes and transplanted intravenously via tail vein with human leukemia cells suspended in HBSS (Gibco, Catalog #14025-092). Two days after leukemia inoculation, mice were intravenously inoculated via tail vein with 15×10e6 TCR+ T cells after being warmed under a heating lamp for 3-5 minutes for tail vein visualization.

Treated mice underwent in vivo bioluminescence imaging, and body weight monitoring twice per week throughout experiment. Mice were anesthetized with inhaled isoflurane (2%) during imaging procedures. Luciferase-based bioluminescent imaging was performed with an IVIS Spectrum system. Animals were imaged following an intraperitoneal injection of 150 mg/kg D-luciferin (Perkin-Elmer, Part #122799) dissolved in phosphate buffered saline (PBS). Animals were imaged five minutes after injection with the camera set to automatic exposure. Images were taken and bioluminescent signal was recorded using Living Image acquisition and analysis software (caliper Life Sciences, Hopkinton, Mass.). Identical regions of interest (ROI) were drawn over each mouse in order to determine total flux value, measured in photons(p)/second(s). Animals were clinically monitored three times per week and were euthanized upon leukemia dissemination and clinical manifestation (weight loss >18%, hind limb paralysis). Body weights decreased due to disease progression.

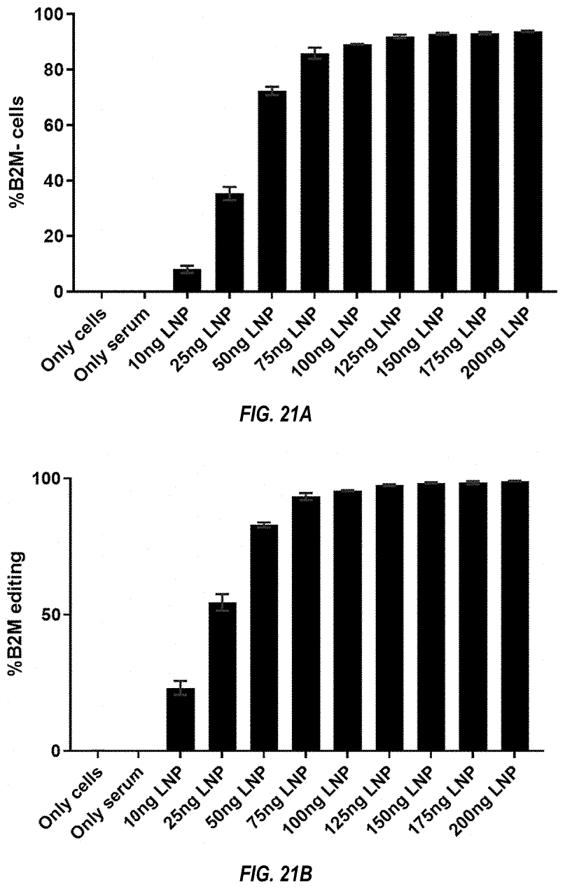

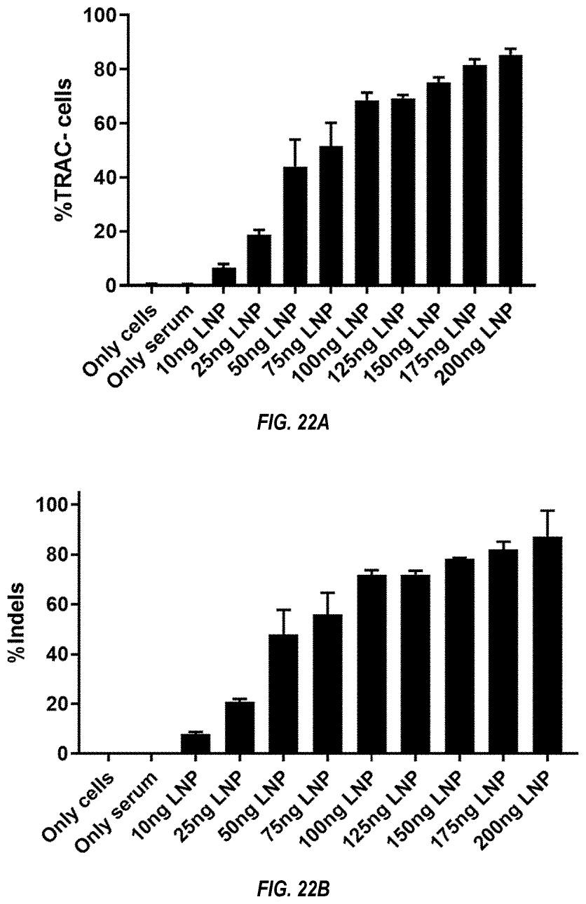

Table 21 shows mean bioluminescence as a measure of ALL liquid tumor burden for all samples and FIG. 19 depicts bioluminescence for NOG-hIL-2 mice, Table 22 shows percent survival for T cell treated mice for all samples and FIG. 20 depicts percent survival for NOG-hIL-2 mice.

TABLE 21
Bioluminescence

| | | Mean Bioluminescence (Tumor Burden) | | | | | | | |
|---|---|---|---|---|---|---|---|---|---|
| | | WT1-tgTCR | | | | GFP-Control T cells | | | |
| | | NOG- | NOG- | | | NOG- | NOG- | | |
| | | h | h | | NOG- | h | h | | NOG- |
| | | IL-2 | IL-15 | NOG | EXL | IL-2 | IL-15 | NOG | EXL |
| | N | 7 | 7 | 7 | 5 | 7 | 7 | 7 | 5 |
| Days | 1 | 2.03E+ | 2.23E+ | 1.48E+ | 1.84E+ | 2.59E+ | 1.55E+ | 1.56E+ | 2.12E+ |
| post | | 06 | 06 | 06 | 06 | 06 | 06 | 06 | 06 |
| ALL | 6 | 1.12E+ | 1.10E+ | 1.21E+ | 1.00E+ | 3.68E+ | 3.89E+ | 6.40E+ | 1.30E+ |
| inocu- | | 06 | 06 | 06 | 06 | 07 | 07 | 07 | 07 |
| lation | 9 | 1.26E+ | 1.23E+ | 1.82E+ | 1.22E+ | 2.21E+ | 2.31E+ | 5.58E+ | 3.96E+ |
| | | 06 | 06 | 06 | 06 | 08 | 08 | 08 | 08 |
| | 14 | 1.43E+ | 1.71E+ | 2.40E+ | 5.42E+ | 1.45E+ | 1.26E+ | 6.20E+ | 8.19E+ |
| | | 06 | 06 | 07 | 06 | 10 | 10 | 09 | 09 |
| | 16 | 1.12E+ | 2.24E+ | 4.76E+ | 6.94E+ | 1.44E+ | 2.41E+ | 2.92E+ | 3.08E+ |
| | | 06 | 06 | 07 | 06 | 10 | 10 | 10 | 10 |
| | 21 | 2.00E+ | 3.20E+ | 6.67E+ | 3.46E+ | 3.76E+ | 3.28E+ | | |
| | | 06 | 07 | 08 | 08 | 10 | 10 | | |
| | 24 | 4.74E+ | 8.19E+ | 2.41E+ | 1.34E+ | | | | |
| | | 06 | 06 | 09 | 09 | | | | |

TABLE 22
Percent survival for T cell treated mice

| | | EF1a-HD1 | | | | GFP-KO | | | |
|---|---|---|---|---|---|---|---|---|---|
| | | NOG- | NOG- | | | NOG- | NOG- | | |
| | | h IL- | h IL- | | NOG- | h IL- | h IL- | | NOG- |
| | | 2 | 15 | NOG | EXL | 2 | 15 | NOG | EXL |
| N | | 7 | 7 | 7 | 5 | 7 | 7 | 7 | 5 |
| Day Post | 0 | 100 | 100 | 100 | 100 | 100 | 100 | 100 | 100 |
| ALL | 2 | 100 | 100 | 100 | 100 | 100 | 100 | 100 | 100 |
| Inoculation | 6 | 100 | 100 | 100 | 100 | 100 | 100 | 100 | 100 |
| | 9 | 100 | 71 | 100 | 100 | 100 | 100 | 100 | 100 |
| | 14 | 100 | 71 | 100 | 100 | 100 | 100 | 100 | 100 |
| | 16 | 86 | 71 | 100 | 100 | 86 | 86 | 43 | 100 |
| | 20 | 86 | 71 | 100 | 100 | 57 | 86 | 43 | 0 |
| | 21 | 86 | 71 | 100 | 100 | 14 | 14 | 0 | |
| | 24 | 86 | 71 | 100 | 100 | 0 | 0 | | |
| | 27 | 86 | 71 | 100 | 100 | | | | |

### Example 5. LNP Dose Response Study in T Cells

T cells were either obtained commercially (e.g. Human Peripheral Blood CD4⁺CD45RA⁺ T Cells, Frozen, Stem Cell Technology, Cat. 70029) or prepared internally from a leukopak. For internal preparation, T cells were isolated by negative selection using the EasySep Human T cell Isolation Kit (Stem Cell Technology, Cat. 17951) following the manufacturers protocol. T cells were cryopreserved in Cryostor CS10 freezing media (Cat. 07930) for future use. Isolated T cells were thawed in in Media Number 11, as described in Table 2. Upon thaw, the cells were activated by addition of 3:1 ratio of CD3/CD28 beads (Dynabeads, Life Technologies) and cultured at 37° C. for 48 hours prior to LNP addition.

Post activation, LNPs delivering Cas9 mRNA and sgRNAs G000529 (SEQ ID NO: 701) and G012086 (SEQ ID NO: 703), targeting B2M and TRAC respectively, were delivered to T cells.

In this example, LNPs were formulated with a cationic lipid amine to RNA phosphate (N:P) molar ratio of about 4.5. The lipid nanoparticle components were dissolved in 100% ethanol with the following molar ratios: 45 mol-% (12.7 mM) cationic lipid (e.g., (9Z,12Z)-3-((4,4bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3 (diethylamino)propoxy)carbonyl)oxy)methyl)propyl (9Z,12Z)-octadeca-9,12-dienoate, referred to herein as Lipid A); 44 mol % (12.4 mM) helper lipid (e.g., cholesterol); 9 mol % (2.53 mM) neutral lipid (e.g., DSPC); and 2 mol % (0.563 mM) PEG (e.g., PEG2k-DMG). The RNA cargo was prepared in 25 mM Sodium citrate, 100 mM NaCl buffer, pH 5, resulting in a concentration of RNA cargo of approximately 0.45 mg/ml. The LNPs were formed by microfluidic mixing of the lipid and RNA solutions using a Precision Nanosystems NanoAssemblr™ Benchtop Instrument, according to the manufacturer's protocol. The formulations were buffer exchanged using PD-10 desalting columns (GE) into 50 mM Tris-HCl, 45 mM NaCl, 5% (w/v) sucrose pH 7.5 (TSS) and filtered through a 0.2 um membrane filter.

LNPs were preincubated at 37° C. for about 5 minutes with M. fascicularis (cynomolgus monkey) serum (BioReclamationlVT, CYN197452) at 6% (v/v). The pre-incubated LNPs were added to T cells at various amounts of total RNA cargo as indicated in Table 23 and Table 24. After 24 hours LNP exposure, cells were washed and transferred to a 24 well plate. Five days post LNP transfection, cells were collected for flow cytometric analysis and NGS sequencing. DNA samples were subjected to PCR and subsequent NGS analysis, as described in Example 1.

### Example 5.1. Flow cytometry analysis

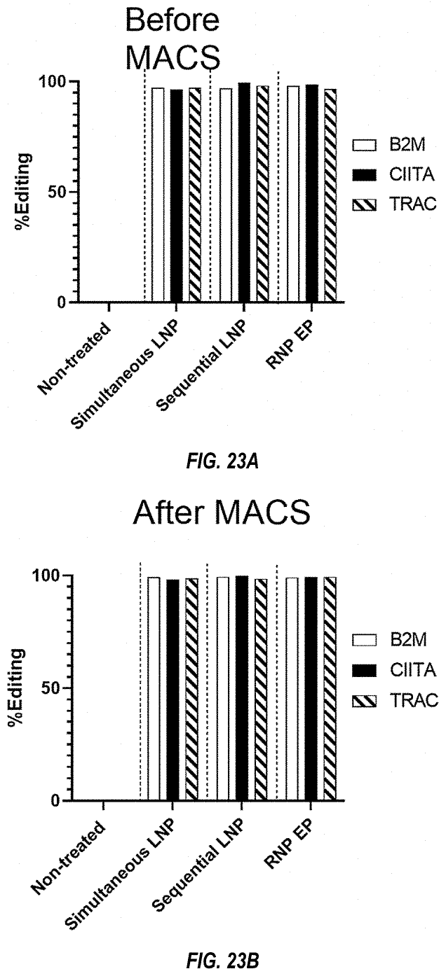

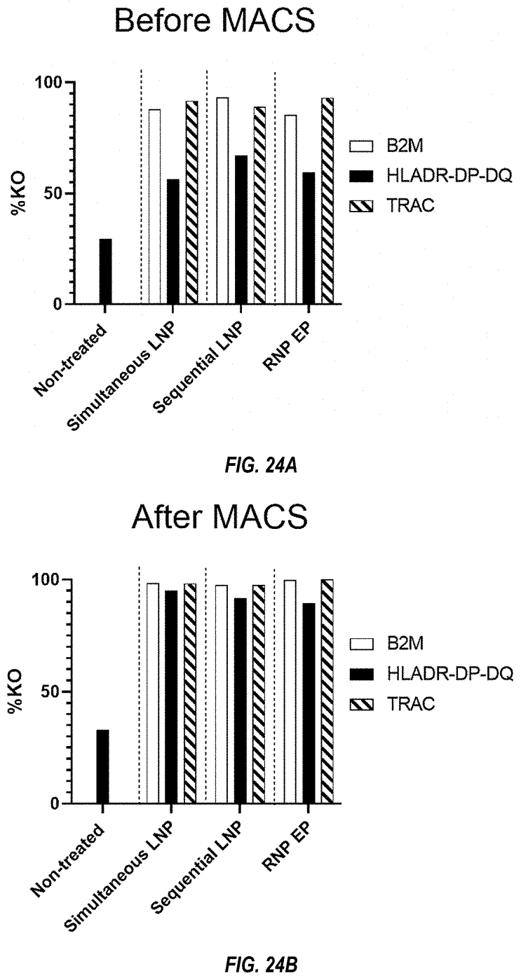

For flow cytometric analysis, cells were washed in FACS buffer (PBS+2% FBS+ 2 mM EDTA). Then the cells were blocked with Human TruStain FcX (Biolegend®, Cat. 422302) at room temperature (RT) for 5 minutes and incubated with APC-conjugated anti-human B2M antibody (Biolegend®, 316312) or PE-conjugated TRAC antibody (Biolegend®, Cat. 304120) at 1:200 dilution for 30 mins at 4° C. After the incubation, the cells were washed and resuspended buffer containing live-dead marker 7AAD (1:1000 dilution; Biolegend®; 420404). The cells processed by flow cytometry, for example using a Beckman Coulter CytoflexS, and analyzed using the FlowJo software package. Table 23 and FIGS. 21A-21B show the percentage of B2M negative cells and percent editing at each LNP dose. Table 24 and FIGS. 22A-22B show the percentage of TRAC negative cells and percent editing at each LNP dose.

TABLE 23
Dose response study of B2M editing

| LNP Dose (ng | Mean % | | Mean % | | |
|---|---|---|---|---|---|
| total RNA) | Edit | SD | B2M negative | SD | N |
| 10 | 23.1 | 2.6 | 8.1 | 1.3 | 3 |
| 25 | 54.5 | 3.0 | 35.3 | 2.4 | 3 |
| 50 | 83.0 | 0.9 | 72.4 | 1.5 | 3 |
| 75 | 93.4 | 1.3 | 86.0 | 2.0 | 3 |
| 100 | 95.5 | 0.2 | 89.2 | 0.1 | 3 |
| 125 | 97.6 | 0.3 | 92.0 | 0.6 | 3 |
| 150 | 98.4 | 0.2 | 93.0 | 0.3 | 3 |
| 175 | 98.5 | 0.6 | 93.1 | 0.4 | 3 |
| 200 | 99.1 | 0.1 | 93.9 | 0.2 | 3 |

TABLE 24
Dose response study of TRAC editing

| LNP Dose | Mean | | | Mean % | | |
|---|---|---|---|---|---|---|
| (ng | % | | | TRAC | | |
| total RNA) | Edit | SD | N | negative | SD | N |
| 10 | 23.1 | 2.6 | 3 | 7.7 | 0.2 | 3 |
| 25 | 54.5 | 3.0 | 3 | 18.6 | 2.0 | 2 |
| 50 | 83.0 | 0.9 | 3 | 44.1 | 9.9 | 3 |
| 75 | 93.4 | 1.3 | 3 | 51.6 | 8.7 | 3 |
| 100 | 95.5 | 0.2 | 3 | 68.6 | 2.8 | 3 |
| 125 | 97.6 | 0.3 | 3 | 69.3 | 1.3 | 3 |
| 150 | 98.4 | 0.2 | 3 | 75.2 | 1.8 | 3 |
| 175 | 98.5 | 0.6 | 3 | 81.5 | 2.3 | 3 |
| 200 | 99.1 | 0.1 | 3 | 85.4 | 2.3 | 3 |

### Example 6. Directional Genomic Hybridization Analysis for Chromosomal Translocation Following Gene Editing

T cells treated with electroporation or lipid nanoparticles to deliver Cas9 mRNA and guides were analyzed for chromosomal structural variations including translocations by directional Genomic Hybridization (dGH™) by KromaTiD (Longmont, Colo.).

### Example 6.1. Electroporation Treatments

For the electroporation treatment, T cells were isolated and cryopreserved as in Example 5. Cryopreserved T cells were thawed and rested overnight in Media Number 1, as described in Table 2.

Rested T cells were electroporated to deliver ribonucleoprotein (RNP) complexes containing guides G013674 (SEQ ID NO: 702) or G000529 (SEQ ID NO: 701), targeting CIITA and B2M genes respectively. Briefly stock RNPs were prepared by incubating recombinant Cas9-NLS protein (50 μM stock) with sgRNA (100 μM) to a final concentration of 20 μM Cas9 with 40 μM sgRNA (1:2 Cas9 protein to guide ratio). Cultured T cells were harvested at 10e6 cells resuspended in 100 μL Buffer P3 (Lonza, Cat. V4SP-3960) and incubated with 12.5 μL of RNPs to a final concentration of 2 μM each. T cells were subsequently electroporated using the Lonza 4D nucleofector 5. Electroporated cells were collected and rested for 48 hours in Media Number 1, as described in Table 2. Subsequently, T cells were harvested, resuspended to a density of 1×10e6 cells/mL in Media Number 1, as described in Table 2 and activated with T cell TransAct reagent (Miltenyi, Cat. 130-111-160) at a 1/100 dilution. Forty-eight hours after T cell activation, T cells were electroporated as described above with Cas9-RNPs including G012086 (SEQ ID NO: 703) targeting TRAC. Triple edited T cells was transferred back to Media Number 1, as described in Table 2 and expanded for future analysis.

After expansion, the cells were passed through the Magnetic-Activated Cell Sorting (MACS) depletion process for selecting the triple knockout cells using the Anti-Biotin microbeads (Miltenyi Biotec, Cat. 130-090-485) protocol for MHC Class I (Miltenyi Biotec, Cat. 130-120-431), MHC Class II (Miltenyi Biotec, 130-104-823) and CD3-biotin (Miltenyi Biotec, Cat. 130-098-612) as per the manufacturer's protocol. The negatively selected cells were collected for flow cytometry analysis and NGS analysis. The protocols described in Example 5 were used for these analyses.

### Example 6.2. Sequential and Simultaneous LNP Treatment

For the LNP treatment, T cells were isolated and cryopreserved as in Example 5. Upon thaw, T cells were activated with T cell TransAct (Miltenyi Biotec, Cat. 130-111-160) as recommended by the manufacturer's protocol and cultured at 37° C. for 24-72 hours as specified below.

For the simultaneous LNP treatment, T cells were treated 72 hours post activation with three LNPs delivering Cas9 mRNA and sgRNAs G000529 (SEQ ID NO: 701), G012086 (SEQ ID NO: 703), and G013674 (SEQ ID NO: 702) targeting B2M, TRAC and CIITA respectively. LNPs were formulated with ionizable lipid nonyl 8-((8,8-bis(octyloxy)octyl)(2-hydroxyethyl)amino)octanoate, referred to herein as Lipid B, as described in Example 1 at a ratio of 50/10/38.5/1.5 ionizable lipid, cholesterol, DSPC, and PEG2k-DMG. LNPs were pre-incubated in 6% cynomologus serum at 37° C. for 5 mins and dosed at 100 ng of total RNA cargo per 100,000 T cells. After 24 hours LNP exposure, the cells were washed and resuspended in Media Number 11, as described in Table 2, and cultured at 37° C. for 5 days.

For sequential LNP treatment, T cells were treated 24 hours post activation with a single LNP delivering Cas9 mRNA and G000529 (SEQ ID NO: 701) targeting B2M as described for simultaneous LNP treatment above. Following wash and resuspension, a single LNP delivering Cas9 mRNA and G013674 (SEQ ID NO: 702) targeting CIITA was added at 48 hours post activation. Lastly, following wash and resuspension, a single LNP delivering Cas9 mRNA and G012086 (SEQ ID NO: 703) targeting TRAC was added at 72 hours post activation. After 24 hours exposure to the final LNP, cells were washed and resuspended in Media Number 11, as described in Table 2, and cultured at 37° C. for 5 days.

LNP treated T cells were passed through the MACS triple negative selection process and further flow cytometry analysis and NGS analysis were performed on these samples as described for electroporation treated cells above.

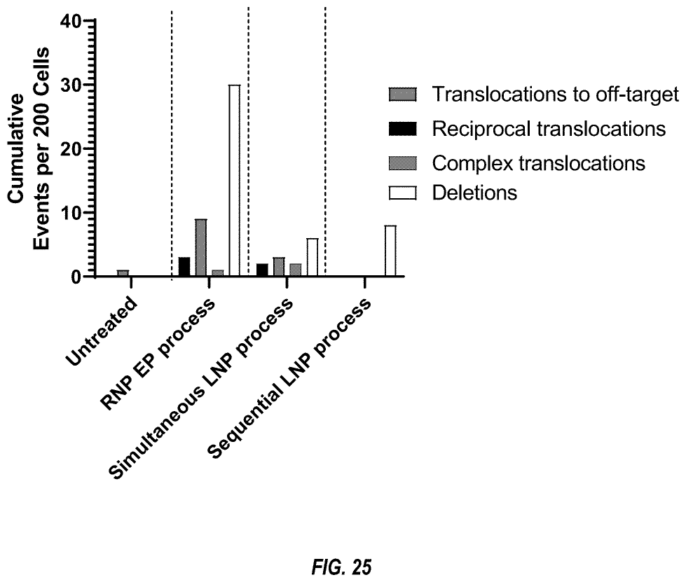

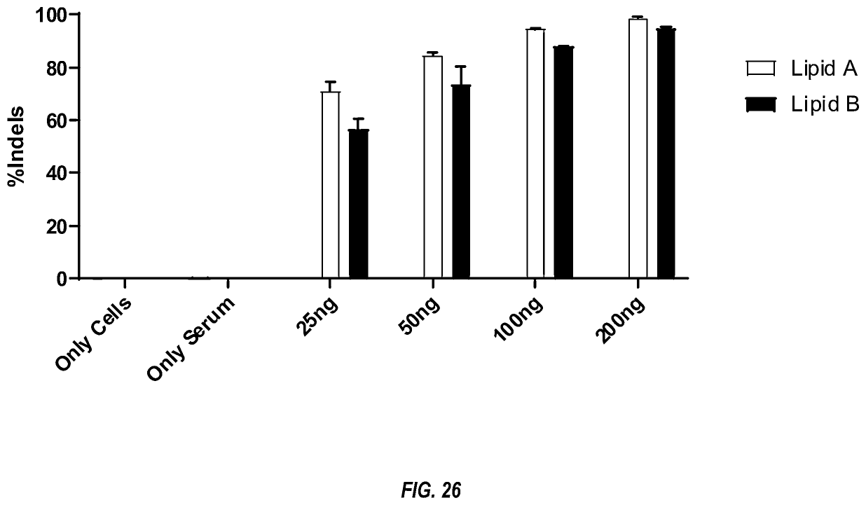

Treated and non-treated cells were assayed for percent editing by NGS and protein expression by flow cytometry as described in Example 5 both before and after MACS processing. The following flow cytometry reagents were used as phenotypic readouts of gene editing for B2M, CIITA and TRAC, respectively: FITC anti-human β2-microglobulin Antibody (Biolegend®, Cat. 316304), APC anti-human CD3 Antibody (Biolegend®, Cat. 300412), PE anti-human HLA-DR, DP, DQ Antibody (Biolegend®, Cat. 361716). NGS editing results are shown in Table 25 and FIGS. 23A-23B. Flow cytometry results are shown in Table 26 and FIGS. 24A-24B. Reduced expression of human MHC class II protein (e.g., HLA-DR, HLA-DP, and HLA-DR) indicates editing of the CIITA gene. CIITA is a transcriptional regulator of MHC class II molecules.

TABLE 25
Editing analysis by NGS

| | Condition | | | | | |
|---|---|---|---|---|---|---|
| | B2M | | CIITA | | TRAC | |
| | % edit | | % edit | | % edit | |
| MACS | Before | After | Before | After | Before | After |
| Non-treated | 0.1 | 0.1 | 0.2 | 0.2 | 0.2 | 0.1 |
| Simultaneous | 97.3 | 99.3 | 96.5 | 98.2 | 97.3 | 98.6 |
| LNP | | | | | | |
| Sequential | 97.0 | 99.4 | 99.6 | 99.8 | 98.2 | 98.5 |
| LNP | | | | | | |
| RNP EP | 98.0 | 99.1 | 98.7 | 99.4 | 96.7 | 99.4 |

TABLE 26
Flow cytometry analysis

| | Condition | | | | | |
|---|---|---|---|---|---|---|
| | | | HLA-DR- | | | |
| | B2M % | | DP-DQ % | | CD3 % | |
| | negative | | negative | | negative | |
| MACS | Before | After | Before | After | Before | After |
| Non-treated | 0.2 | 0.2 | 29.4 | 32.8 | 0.3 | 0.2 |
| Simultaneous | 87.9 | 98.4 | 56.5 | 95.0 | 91.7 | 98.3 |
| LNP | | | | | | |
| Sequential | 93.2 | 97.6 | 67.0 | 91.8 | 89.0 | 97.6 |
| LNP | | | | | | |
| RNP EP | 85.4 | 99.9 | 59.6 | 89.4 | 93.1 | 100 |

### Example 6.3. Kromatid dGH™ Analysis for Chromosomal Structural Rearrangements

Engineered T cells were prepared for the dGH procedure according to the KromaTiD's protocol. Briefly, T cells were cultured for 17 hours with the addition of 5 BrdU and 1 μM BrdC as provided by KromaTiD. Colcemid was added at a concentration of 10 μl/m1 for an additional 4 hours. Cells were harvested by centrifugation, incubated in 75 mM KCl hypotonic solution for 30 minutes at room temperature, and fixed in a 3:1 methanol to acetic acid solution.

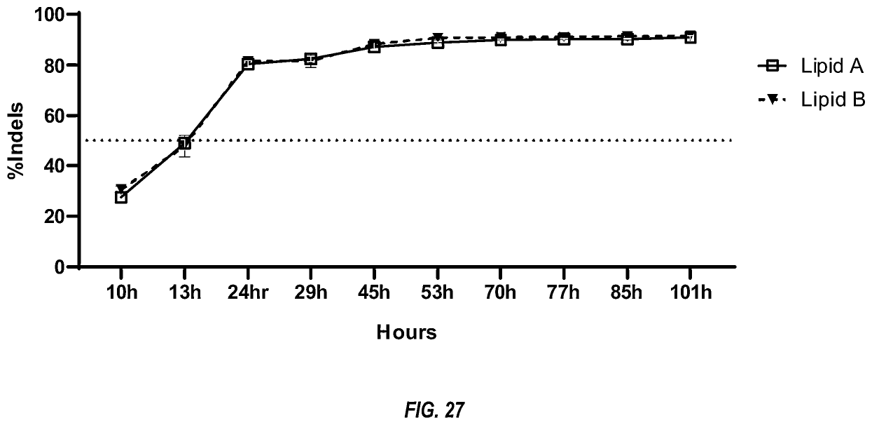

Three sets of fluorescence in situ hybridization (FISH) probes were designed to bracket the genomic target sites of the guides used to engineer these T cells, which are located on separate chromosomes. KromaTiD imaged 200 metaphase spreads per sample using their proprietary dGH FISH and scored the spreads for chromosomal structural rearrangements. Cells without chromosomal structural rearrangements showed 3 matched-color, adjacent pairs of FISH signals. “Deletions” were scored when zero FISH signals for a target site were identified in the cell, indicating chromosomal rearrangement where fragments were lost during the cell replication cycle due to the editing event occurring. “Reciprocal translocations” were scored for each pair of adjacent, color-mismatched FISH signals, indicating a translocation between two Cas9-targeted cleavages (e.g. between B2M and TRAC target sites). “Translocations to off-target chromosomes” showed a single FISH signal, indicating a fusion between a Cas9-targeted cleavage site and unlabeled chromosomal site. “Complex translocations” denote FISH signals not included in reciprocal translocations and translocations to off-target sites. Total translocations were calculated as a sum total of the reciprocal translocations, translocations to off-target chromosomes/sites in the genome and complex translocations. Table 27 and FIG. 25 show the chromosomal rearrangements identified by this method for each condition.

TABLE 27
Translocations analysis by Kromatid dGH assay

| Chromosomal | | | | |
|---|---|---|---|---|
| rearrangements | | Sequential | Simultaneous | RNP |
| events: | Untreated | LNP | LNP | EP |
| Total | 1 | 0 | 7 | 13 |
| Translocations | | | | |
| Reciprocal | 0 | 0 | 2 | 3 |
| translocations | | | | |
| Translocations to | 1 | 0 | 3 | 9 |
| off-target | | | | |
| chromosomes | | | | |
| Complex | 0 | 0 | 2 | 1 |
| Translocations | | | | |
| Deletions | 0 | 8 | 6 | 30 |

### Example 7. LNP Delivery to T Cell with Different Ionizable Lipid Formulations

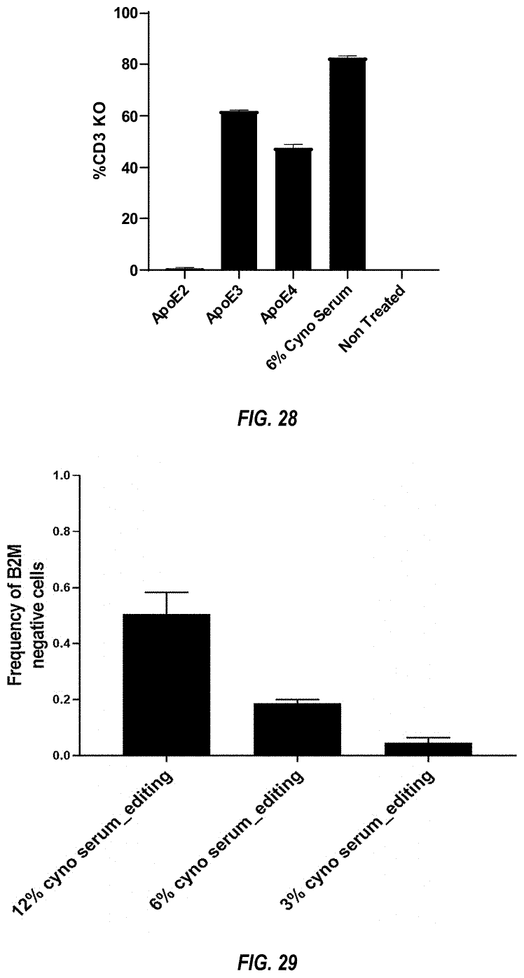

LNPs formulated with different ionizable lipids were tested for T cell delivery efficacy. T cells were prepared, thawed and activated as in Example 5. Forty-eight hours post activation, T cells were treated with LNPs delivering Cas9 mRNA and gRNA G000529 (SEQ ID NO: 701) targeting B2M. LNPs were generally prepared as Example 1. Lipid A formulations were prepared at a ratio of 50/9/38/3 ionizable Lipid A, cholesterol, DSPC, and PEG2k-DMG. Lipid B compositions were formulated at a ratio of 50/10/38.5/1.5 ionizable lipid nonyl 8-((8,8-bis(octyloxy)octyl)(2-hydroxyethyl)amino)octanoate, cholesterol, DSPC, and PEG2k-DMG. LNPs were preincubated at 37° C. for about 5 minutes with M. fascicularis (cynomolgus monkey) serum (BioreclamationlVT, CYN197452) at 3% final (v/v). The pre-incubated LNPs were added to T cells at amounts of total RNA cargo as indicated in Table 28. After 24 hours LNP exposure, cells were washed and transferred to a 24 well plate. Five days post LNP treatment, cells were collected and NGS analysis was performed as described in Example 1. Efficient editing was evident using LNPs formulated with both Lipid A and Lipid B, as shown in FIG. 26 and Table 28.

TABLE 28
Mean editing percentage of dose response study by NGS

| Sample | Dose (ng total RNA) | Mean % Edit | SD | N |
|---|---|---|---|---|
| Only Cells | 0 | 0.1 | 0.1 | 3 |
| Only Serum | 0 | 0.2 | 0.1 | 3 |
| Lipid A | 25 | 71.1 | 3.5 | 3 |
| | 50 | 84.7 | 0.9 | 2 |
| | 100 | 94.4 | 0.2 | 3 |
| | 200 | 98.6 | 0.6 | 3 |
| Lipid B | 25 | 56.4 | 4.1 | 3 |
| | 50 | 73.6 | 6.6 | 2 |
| | 100 | 87.9 | 0.2 | 3 |
| | 200 | 94.9 | 0.4 | 3 |

### Example 8. Editing Kinetics for LNP-Engineered T Cells

To determine minimum LNP exposure time for maximum editing in LNP-engineered T cells, percent indel rates at different time points post LNP contact were determined.

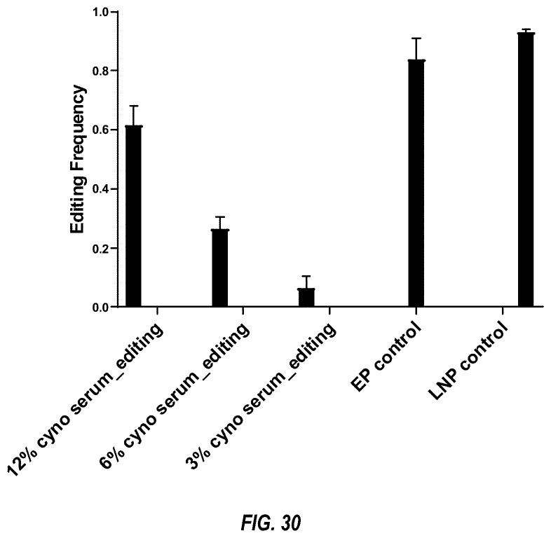

CD3+ T cells were prepared, thawed, and activated as described in Example 5. Post activation, LNPs delivering Cas9 mRNA and sgRNA G000529 (SEQ ID NO: 701) targeting B2M were delivered to T cells. LNPs were generally prepared as Example 1. Lipid A LNPs were prepared at a ratio of 50/9/38/3 ionizable lipid, cholesterol, DSPC, and PEG2k-DMG. The Lipid B LNPs were formulated at a ratio of 50/10/38.5/1.5 ionizable lipid B, cholesterol, DSPC, and PEG2k-DMG. LNPs were pre-incubated with 6% cynomolgus (cyno) serum (v/v) at 37° C. for 60 minutes. The pre-incubated LNPs were dosed at fifty nanograms of total RNA cargo on to T cells. At time points post LNP contact as indicated in Table 31, 250 μL of T cells were collected and analyzed by NGS as described in Example 1. Editing results at each time point are shown in Table 29 and FIG. 27.

TABLE 29
Editing kinetic study NGS editing data

| | Time from LNP | Mean % | | |
|---|---|---|---|---|
| Sample | addition (hours) | Editing | SD | N |
| Lipid A | 10 | 27.5 | 1.0 | 3 |
| | 13 | 49.0 | 2.5 | 3 |
| | 24 | 80.3 | 1.2 | 3 |
| | 29 | 82.5 | 1.1 | 3 |
| | 45 | 87.0 | 2.0 | 3 |
| | 53 | 88.8 | 0.4 | 3 |
| | 70 | 89.9 | 0.3 | 3 |
| | 77 | 90.1 | 0.5 | 3 |
| | 85 | 90.3 | 0.2 | 3 |
| | 101 | 90.9 | 0.6 | 3 |
| Lipid B | 10 | 30.6 | 1.5 | 3 |
| | 13 | 47.8 | 4.3 | 3 |
| | 24 | 81.4 | 1.1 | 3 |
| | 29 | 81.6 | 2.5 | 3 |
| | 45 | 88.2 | 0.5 | 3 |
| | 53 | 90.7 | 0.7 | 3 |
| | 70 | 90.9 | 0.4 | 3 |
| | 77 | 91.0 | 0.3 | 3 |
| | 85 | 91.3 | 0.2 | 3 |
| | 101 | 91.4 | 1.1 | 3 |

### Example 9. LNP Delivery to T Cells with Various Serum Factor Sources

T cells were engineered with LNPs preincubated with various sources of serum or recombinant ApoE isoforms. The study was performed as an 8-point dose response assay using human serum, cyno serum and ApoE isoforms ApoE2, ApoE3 and ApoE4.T cells were prepared and cryopreserved as in Example 5. Upon thaw in Media Number 1, as described in Table 2, T cells were activated with TransAct (1:100 dilution, Milteni Biotec, Catalog #130-111-160) for 48 hours prior to editing.

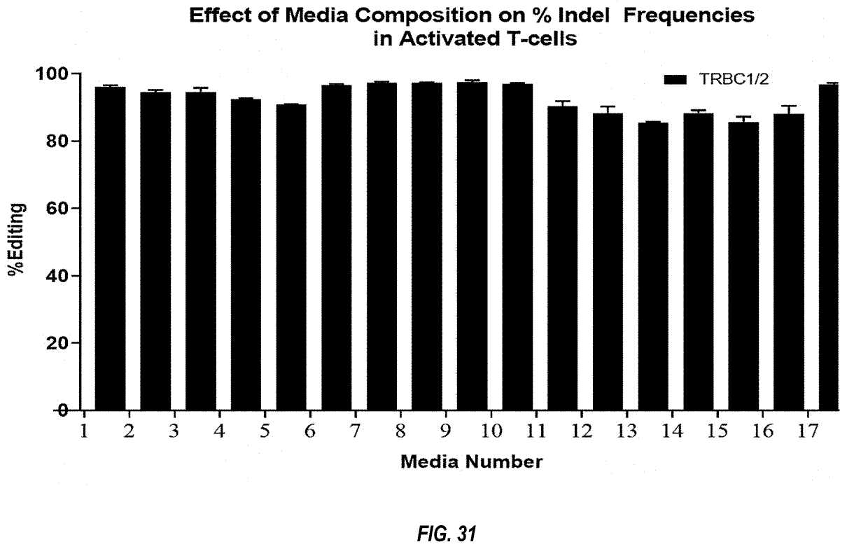

Post activation, LNPs delivering Cas9 mRNA and sgRNAs targeting TRAC (G013006, SEQ ID NO: 708) were delivered to T cells. LNPs were formulated as described in Example 1 with a ratio of gRNA to mRNA of 1:2 by weight. LNPs were preincubated at 37° C. for about 5 minutes with the different ApoE isoforms ApoE2 (Biovision, Cat. #4760), ApoE3 (R&D systems, Catalog #4144-AE-500) and ApoE4 (Novus Biologicals, Catalog #NBP1-99634) as described in Table 30. The pre-incubated LNPs were added to T cells at 100 ng of total RNA cargo. Five days post LNP transfection, cells were collected for flow cytometric analyses as described in Example 5. Results are shown in FIG. 28 and Table 30. Reduced expression of MEC class I protein (e.g., HLA-A, HLA-B, and HLA-C) indicates editing of the B2M gene. The B2M protein is a component of MEC class I proteins, therefore, if B2M is knocked out, MEC class I protein will not be detected.

TABLE 30
Mean % CD3 KO in different doses of ApoE isoforms

| | Dose | Mean % CD3 | Standard |
|---|---|---|---|
| Sample | (μg/mL) | negative cells | Deviation |
| 6% cyno | n/a | 82.46 | 0.74 |
| serum | | | |
| Untreated | 0 | 0.00 | 0.00 |
| ApoE2 | 1 | 0.70 | 0.16 |
| | 0.5 | 0.72 | 0.09 |
| | 0.25 | 0.98 | 0.20 |
| | 0.125 | 1.30 | 0.25 |
| | 0.0625 | 2.69 | 0.23 |
| | 0.0312 | 0.81 | 0.18 |
| | 0.015 | 1.26 | 0.03 |
| | 0.007 | 1.97 | 0.59 |
| ApoE3 | 1 | 61.87 | 0.38 |
| | 0.5 | 56.33 | 1.19 |
| | 0.25 | 67.80 | 0.28 |
| | 0.125 | 70.50 | 0.29 |
| | 0.0625 | 70.83 | 2.36 |
| | 0.0312 | 71.77 | 4.37 |
| | 0.015 | 67.90 | 4.68 |
| | 0.007 | 69.47 | 4.01 |
| ApoE4 | 1 | 47.40 | 1.31 |
| | 0.5 | 66.23 | 0.49 |
| | 0.25 | 67.50 | 0.86 |
| | 0.125 | 69.03 | 1.10 |
| | 0.0625 | 72.53 | 0.87 |
| | 0.0312 | 73.73 | 0.59 |
| | 0.015 | 64.93 | 3.14 |
| | 0.007 | 60.30 | 2.12 |

### Example 10. Lipoplex Treatment with Various Concentrations of Serum for Lipofection Delivery to T Cells

To determine the conditions for high efficiency lipoplex delivery to T cells, mRNA encoding SpyCas9 along with single guide RNA was delivered by using lipofection reagent.

### Example 10.1. Cell Culture

Healthy human donor PBMCs or leukopaks were obtained commercially (Hemacare) and T cells were isolated by CD4/CD8 positive selection using the StraightFrom® Leukopak® CD4/CD8 MicroBeads (Milteni Biotec, Catalog #130-122-352) following the manufacturers protocol on MultiMACS™ Cell24 Separator Plus instrument. T cells were aliquoted into vials and cryopreserved in Cryostor CS10 freezing media (Catalog #07930) for future use. Vials were subsequently thawed as needed for experiments. These T cells were then thawed in water bath and transferred into 10 mL of prewarmed Media Number 5, as described in Table 2. Upon thaw, T cells were activated by addition of 1:100 dilution of TransAct (Miltenyi Biotech, Cat. 130-111-160) in Media Number 1, as described in Table 2. The cells are activated for 48 hours at 37° C. before T cell engineering treatment.

### Example 10.2. Lipofection of Human T Cells

After 48 hours of T cell culturing, T cells were treated in biological replicates with lipoplexes. Lipofection reagent was prepared as mixture of lipids at a ratio of 50/9/38/3 Lipid A, cholesterol, DSPC, and PEG2k-DMG as described in Example 1. Lipofection reagent was combined by bulk mixing with Cas9mRNA and gRNA G000529 (SEQ ID NO: 701) targeting B2M. The materials were combined at a lipid amine to RNA phosphate (N:P) molar ratio of about 6, and a w/w ratio of mRNA to gRNA of 1:2. The resulting bulk-mixed lipoplex material (lipid kits) was pre-incubated with 12%, 6%, 3% or 0% cyno serum (Bioreclamation IVT; CYN220760) in Media Number 1, as described in Table 2 for 15 min before addition to T cells.

T cells were treated with lipoplex in biological duplicates at dose of 100 ng of Cas9 mRNA with 200 ng guide sgRNA per 100,000 T cells. T cells were washed 48 hours post lipoplex contact and replaced with fresh complete T cell media. Four days post lipofection half of the cells were collected for NGS sequencing and a day later the other half of the cells for flow cytometry analyses.

### Example 10.3. LNP Treatment of Human T Cells

For transfection control, LNP formulation containing Cas9 mRNA and gRNA G000529 (SEQ ID NO: 701) was added to 100,000 activated T cells. The LNP was preincubated at 37° C. for about 15 minutes with non-human primate serum at 6% (v/v) (M. fascicularis (cynomolgus monkey) serum, BioReclamationlVT, CYN220760) and Media Number 1, as described in Table 2. The pre-incubated LNPs were added to T cells at 100 ng of total RNA cargo (1:2 w/w ratio of Cas9 mRNA and single guide). Cells were washed 48 hours post LNP treatment and replaced with Media Number 1, as described in Table 2. Four days post LNP treatment half of the cells were collected for NGS sequencing analyses.

### Example 10.4. Electroporation of Human T Cells

For electroporation control, RNP was electroporated into 100,000 activated T cells. RNP was formed at a 20 uM stock concentration by mixing Cas9 protein with heat denatured gRNA G000529 (SEQ ID NO: 701) targeting B2M at a 2:1 guide: Cas9 ratio for 15 minutes. Forty-eight hours post activation, T cells were harvested, centrifuged, and resuspended at a concentration of 10×10e6 T-cells/100 uL in P3 electroporation buffer (Lonza). The cell suspension was mixed with RNPs to achieve a final RNP concentration of 2 uM, before being transferred to a Nucleofector plate and electroporated using manufacturer's pulse code. Electroporated T cells were immediately rested in 100 uL Media Number 1, as described in Table 2. Four days post LNP treatment half of the cells were collected for NGS sequencing analyses.

### Example 10.5. NGS and Flow Cytometry

Four days post treatment, T cells were lysed for NGS analysis which was conducted as described in Example 1. Five days post T cells treatment, T cells were phenotyped by flow cytometry to determine B2M protein knockout. Briefly, T cells were incubated in antibody targeting B2M (Anti-human B2M Antibody FITC Labelled, Catalog #316304, Biolegend®). Cells were subsequently washed, analyzed on a CytoFLEX S instrument (Beckman Coulter) using the FlowJo software package. T cells were gated on size, B2M FITC expression.

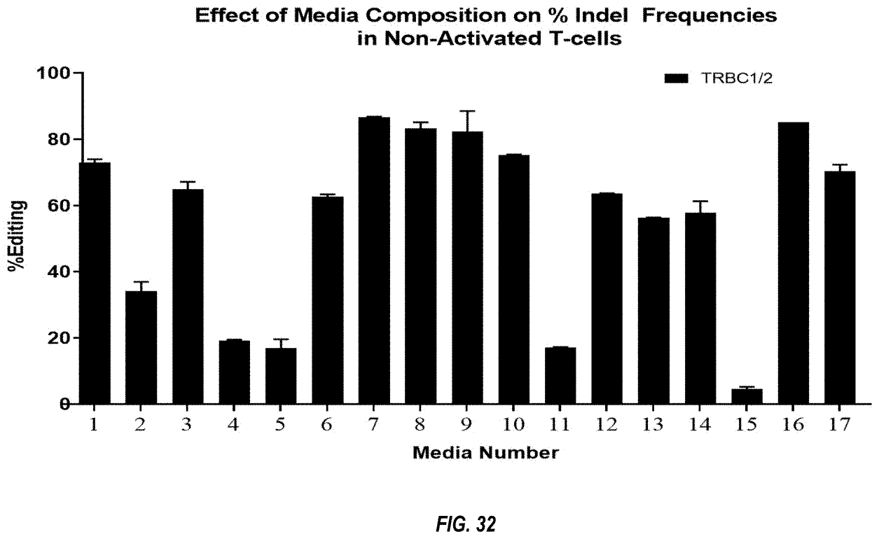

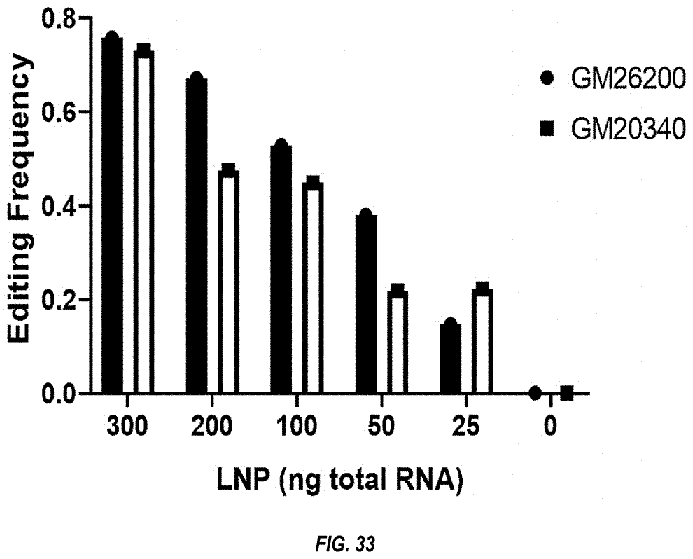

B2M protein knockout frequencies are shown in Table 31 and FIGS. 29, and B2M indel frequencies are shown in Table 32 and FIG. 30.

TABLE 31
B2M protein knockout frequencies with lipid kit treated T cells with
100 ng Cas9 mRNA and 200 nM gRNA.

| | Protein(B2M) Knockout Frequency | |
|---|---|---|
| Serum Conditions | Biological Replicate 1 | Biological Replicate2 |
| 12% cyno serum | 0.56 | 0.448 |
| 6% cyno serum | 0.196 | 0.177 |
| 3% cyno serum | 0.032 | 0.059 |
| Electroporation Control | 0.865 | 0.894 |
| LNP Control | 0.92 | 0.931 |

TABLE 32
B2M indel frequencies with lipid kit treated T cells with 100
ng Cas9 mRNA and 200 nM gRNA

| | B2M Indel Frequency | |
|---|---|---|
| Serum Conditions | Biological Replicate 1 | Biological Replicate2 |
| 12% cyno serum | 0.663 | 0.568 |
| 6% cyno serum | 0.293 | 0.234 |
| 3% cyno serum | 0.033 | 0.092 |

### Example 11. Editing Efficiency in Activated and Non-Activated T Cells

To determine the conditions for high efficiency LNP delivery to both activated and non-activated T cells, deep sequencing was used to assay editing efficiency in T cells following delivery of Cas9 mRNA and sgRNA. T cells were cultured as follows under the conditions listed in Table 33.

### Example 11.1. Cell culture

Healthy human donor PBMCs or leukopaks were obtained commercially (Hemacare) and T cells were isolated by CD4/CD8 positive selection using the StraightFrom® Leukopak® CD4/CD8 MicroBeads (Milteni, Catalog #130-122-352) following the manufacturers protocol on MultiMACS™ Cell24 Separator Plus instrument. T cells were aliquoted into vials and cryopreserved in Cryostor CS10 freezing media (Catalog #07930) for future use. T cells were thawed in Media Number 5 as described in Table 2.

Upon thaw, T cells were activated by addition of 1:100 dilution of TransAct (Milteny Biotech, Catalog #130-111-160) or left non-activated in T cell media as described in Table 2. T cells were cultured at 37 C for 24-48 hr prior to LNP treatment.

### Example 11.2. LNP Treatment and Effect of Culture Media on Human T Cells

Twenty-four hours after initial culture, T cells were treated with an LNP containing Cas9 mRNA and guide G016239 (SEQ ID NO: 707) targeting TRBC. LNPs were prepared with a ratio of gRNA to mRNA of 1:2 by weight. The LNPs were preincubated at 37° C. for about 5 minutes with non-human primate serum at 6% (v/v) (M. fascicularis (cynomolgus monkey) serum, BioreclamationIVT, CYN220760) for a final 3% (v/v) on cells.

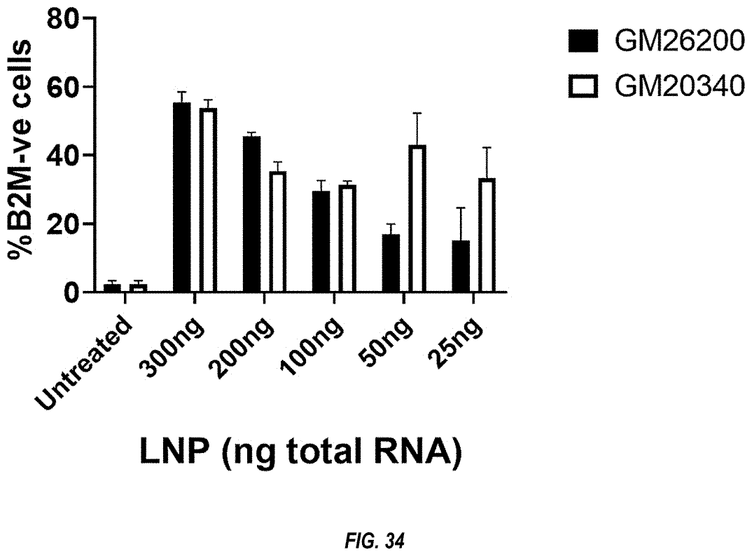

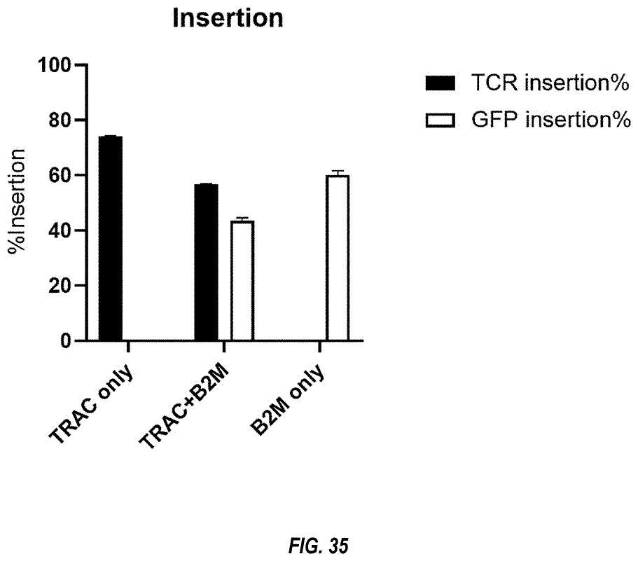

The pre-incubated LNPs were added to T cells at a dose of 100 ng of total RNA cargo in biological replicates. Cells were washed 48 hours post LNP treatment with respective T cells media and replaced with respective fresh T cells media. Five days post LNP treatment, cells were collected for flow cytometric analyses and NGS sequencing. Table 33 show results for indel frequency following editing at both TRBC1 & TRBC2 cut sites in activated T cells. Table 34 results for indel frequency following editing at both TRBC1 & TRBC2 cut sites in non-activated T cells. Effect of media composition on editing is shown in FIG. 31 and FIG. 32.

TABLE 33
Effect of media composition on % indels in activated T cells

| Media Number | % Editing | | | |
|---|---|---|---|---|
| (as described | TRBC1 | | TRBC2 | |
| in Table 2) | Replicate A | Replicate B | Replicate A | Replicate B |
| 1 | 95.5 | 94.8 | 96.4 | 95.8 |
| 2 | 94.4 | 91.8 | 95 | 94.2 |
| 3 | 93.5 | 94.2 | 95.5 | 93.8 |
| 4 | 90.6 | 92 | 92.4 | 92.7 |
| 5 | 88.6 | 89.4 | 91 | 90.9 |
| 6 | 95.4 | 95.4 | 96.9 | 96.5 |
| 7 | 96.5 | 96.3 | 97.1 | 97.6 |
| 8 | 96.5 | 95.4 | 97.3 | 97.4 |
| 9 | 95.3 | 94.9 | 97.1 | 97.9 |
| 10 | 94.9 | 94.6 | 97.2 | 96.7 |
| 11 | 88.2 | 84.4 | 91.4 | 89.2 |
| 12 | 86 | 85.4 | 87 | 89.7 |
| 13 | 84.9 | 83.7 | 85.7 | 85.3 |
| 14 | 84.5 | 83.2 | 87.8 | 88.9 |
| 15 | 85 | 82.4 | 86.8 | 84.6 |
| 16 | 87.3 | 86.1 | 89.8 | 86.5 |
| 17 | 94.8 | 94.5 | 97.1 | 96.4 |

TABLE 24
Effect of media composition on % indels in non-activated T cells

| Media Number | % Editing | | | |
|---|---|---|---|---|
| (as described | TRBC1 | | TRBC2 | |
| in Table 2) | Replicate A | Replicate B | Replicate A | Replicate B |
| 1 | 73.4 | 76.5 | 73.7 | 72.3 |
| 2 | 37.6 | 39.5 | 32.1 | 36.1 |
| 3 | 60.8 | 64.8 | 63.3 | 66.5 |
| 4 | 21.3 | 13 | 19.1 | 19.4 |
| 5 | 15.3 | 25.3 | 18.8 | 14.9 |
| 6 | 55.7 | 58.1 | 62.2 | 63.2 |
| 7 | 89 | 87.1 | 86.7 | 86.8 |
| 8 | 81.2 | 82.9 | 82 | 84.6 |
| 9 | 81.1 | 85.7 | 77.9 | 86.7 |
| 10 | 68.1 | 67.8 | 75.4 | 75.3 |
| 11 | 11.1 | 12 | 17 | 17.2 |
| 12 | 43.3 | 44.8 | 63.4 | 63.7 |
| 13 | 31.7 | 26.2 | 56.2 | 56.4 |
| 14 | 52.4 | 63.5 | 55.3 | 60.3 |
| 15 | 2.2 | 3 | 4.1 | 5 |
| 16 | | | | 85.1 |
| 17 | | 86.7 | 71.8 | 69.1 |

### Example 12. LNP Delivery to Lymphoblastoid Cell Lines

Lipid nanoparticles targeting B2M were used to edit two lymphoblastoid cell lines (LCLs). LCLs are developed by infecting peripheral blood lymphocytes (PBL) from human donors with Epstein Barr Virus (EBV). This process has been demonstrated to immortalize human resting B cells in vitro giving rise to an actively proliferating B cell population positive for B cell marker CD19 and negative for T cell marker CD3 as well as for NK cell marker CD56 (Neitzel H. A routine method for the establishment of permanent growing lymphoblastoid cell lines. Hum Genet. 1986; 73(4):320-6).

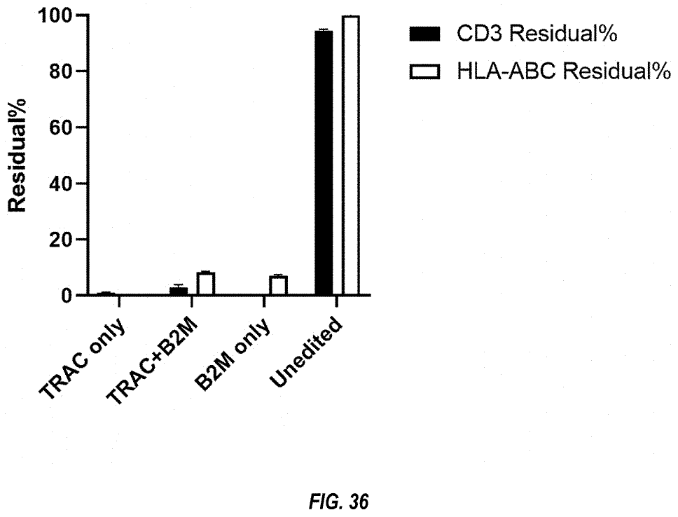

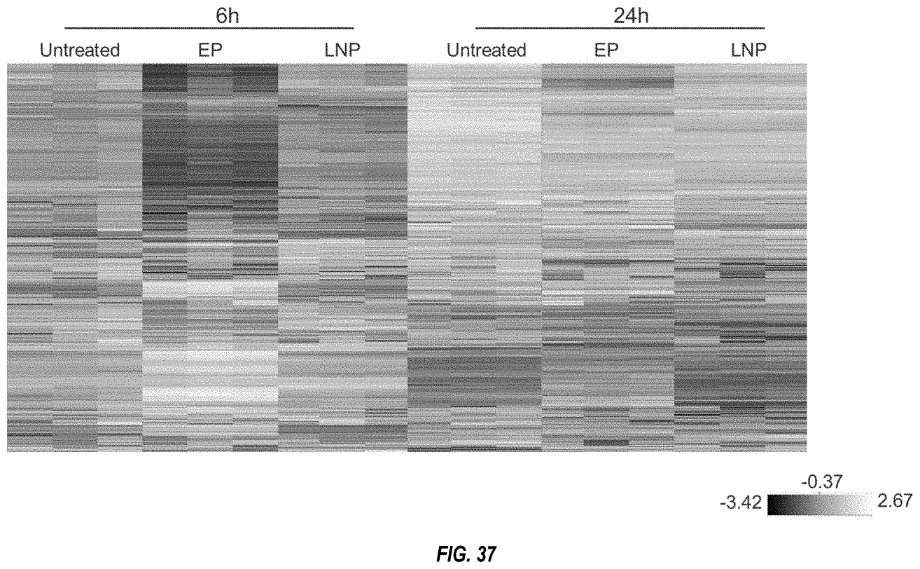

Lymphoblastoid cell lines GM26200 and GM20340 were obtained from the Coriell Institute for Medical Research (Camden, N.J., USA). LCLs were grown in RPMI-1640 with L-glutamine and 15% FBS. At the time of LNP contact, cells were activated with 4 ng/ml IL-4 (R&D System Catalog #204-IL-010), 1 ng/ml IL-40 (R&D System Catalog #6245-CL-050), 25 ng/ml BAFF (R&D System Catalog #2149-BF-010). The LNP was formulated at a ratio of 50/10/38.5/1.5 ionizable Lipid B (nonyl 8-((8,8-bis(octyloxy)octyl)(2-hydroxyethyl)amino)octanoate), cholesterol, DSPC, and PEG2k-DMG as described in Example 1. LNPs formulated with Cas9 mRNA and gRNA G000529 (SEQ ID NO: 701) targeting B2M were pre-incubated with 6% cynomologus serum (v/v) as described in Example 5 and delivered to lymphoblastoid cells at the doses indicated in Table 35 and Table 36. Media was changed every 2 days. Six days post LNP treatment, half of the cells were collected for NGS sequencing and a day later the other half of the cells for flow cytometry analyses. NGS analysis was performed as in Example 1. Flow cytometry was performed as in Example 5 using anti-human B2M antibody (Biolegend (Catalog #316312). Table 35 and FIG. 33 show editing by LNP in two LCLs. Table 36 and FIG. 34 show the percentage of B2M negative cells after LNP treatment.

TABLE 35
Dose response
study of B2M editing in LCLs

| | GM26200 | | | GM20340 | | |
|---|---|---|---|---|---|---|
| Dose | Mean | | | Mean | | |
| (ng total | % | | | % | | |
| RNA) | Edit | SD | N | Edit | SD | N |
| Untreated | 0.0% | 0.0% | 3 | 0.0% | 0.0% | 3 |
| 300 | 75.0% | 0.9% | 3 | 70.4% | 2.6% | 3 |
| 200 | 67.6% | 0.6% | 2 | 46.1% | 1.5% | 3 |
| 100 | 50.3% | 2.3% | 3 | 44.6% | 0.9% | 3 |
| 50 | 34.5% | 3.3% | 3 | 21.8% | 0.2% | 3 |
| 25 | 21.1% | 5.4% | 3 | 21.3% | 1.2% | 3 |

TABLE 36
Dose response study of B2M
protein expression after editing in LCLs

| | GM26200 | | | GM20340 | | |
|---|---|---|---|---|---|---|
| Dose | Mean % | | | Mean % | | |
| (ng total | B2M | | | B2M | | |
| RNA) | negative | SD | N | negative | SD | N |
| Untreated | 2.4 | 1.0 | 3 | 2.4 | 1.0 | 3 |
| 300 | 55.4 | 3.1 | 3 | 53.7 | 2.4 | 3 |
| 200 | 45.5 | 1.2 | 3 | 35.3 | 2.7 | 3 |
| 100 | 29.5 | 3.1 | 3 | 31.4 | 1.2 | 3 |
| 50 | 16.8 | 3.2 | 3 | 43.0 | 9.2 | 3 |
| 25 | 15.1 | 9.5 | 3 | 33.3 | 8.9 | 3 |

### Example 13. Engineering T Cells with Multiple Insertions

T cells were engineered first to knock out protein expression at the TRBC loci followed by simultaneous insertion of a tgTCR to the TRAC locus and GFP to the B2M locus.

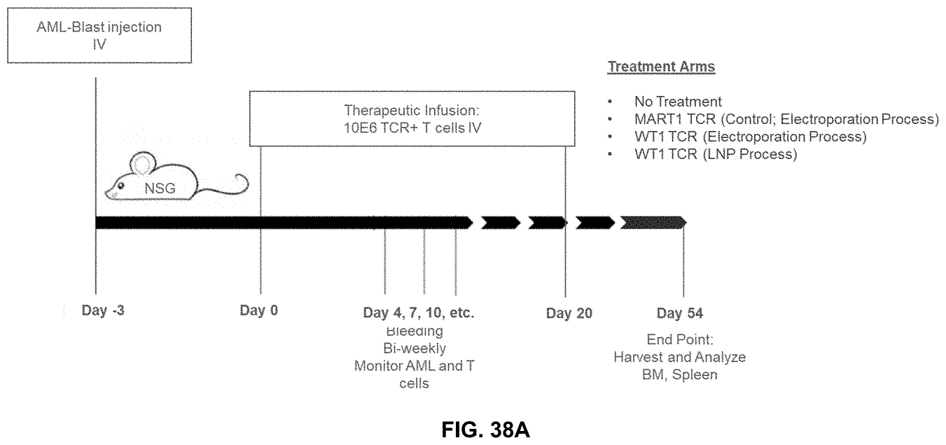

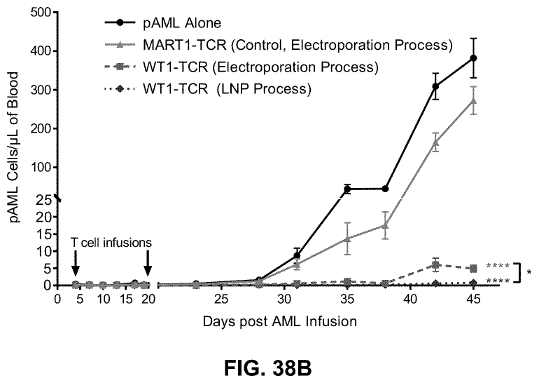

T cells were isolated and cultured in Media Number 17 as described in Table 2. LNPs were generally prepared as described in Example 1. TRAC and TRBC LNPs were prepared at a ratio of 50/9/39.5/1.5 Lipid A, cholesterol, DSPC, and PEG2k-DMG. B2M LNP were prepared at a ratio of 50/10/38.5/1.5 Lipid A, cholesterol, DSPC, and PEG2k-DMG. LNPs were prepared with a ratio of gRNA to mRNA of 1:2 by weight. LNP containing Cas9 mRNA and gRNA G016239 (SEQ ID NO: 707) targeting TRBC was preincubated at a concentration of 5 μg/mL in Media Number 17 as described in Table 2, supplemented with recombinant human ApoE3 (Peprotech, Cat. 350-02) at a concentration of 1 μg/mL for 15 minutes at 37° C. T cells were treated with LNPs targeting TRBC and activated as described for LNP BF2.5 in Example 3. On day 3, these edited T cells were treated with TRAC LNP with gRNA G013006 (SEQ ID NO: 708) and B2M LNP with gRNA G000529 (SEQ ID NO: 701) which were pre-incubated with recombinant human ApoE3 (Peprotech, Cat. 350-02) at a concentration of 20 μg/mL for 15 minutes at 37° C. as described in Example 3. Two HDRT templates were delivered via AAV6 at 300,000 MOI to cells. One HDRT construct contained a WT1 targeting tgTCR with homology arms flanking the TRAC guide cut site. The other HDRT construct contained a GFP sequence with homology arms flanking the B2M guide cut site. Twenty-four hours post LNP and AAV addition, T cells were washed and re-suspended in Media Number 17 as described in Table 2, and expanded in a GREX plate. Six days following treatment and growth, T cells were harvested and assayed by flow cytometry as described in Example 1 using antibodies targeting CD3 (APC-Cy7, Biolegend, Cat. 300318), Vb8 (PE, Biolegend, Cat. 348104), HLA-ABC (BV605, Biolegend, Cat. 311432), CD4 (APC, Biolegend, Cat. 300537) and CD8 (PE/Cy7, Biolegend, Cat. 344712). Table 37 and FIG. 35 show insertion rates. Table 38 and FIG. 36 show percentage of treated cells with residual endogenous protein following insertion.

TABLE 37
Percentage of treated cells with tgTCR insertion and GFP rates

| | | TCR | GFP | GFP |
|---|---|---|---|---|
| | TCR insertion | insertion | insertion | insertion |
| Group | % (mean) | % (SD) | %(mean) | %(SD) |
| TRAC | 74.1 | 0.2 | N/A | N/A |
| TRAC+B2M | 56.8 | 0.1 | 43.5 | 1.1 |
| B2M | N/A | N/A | 60.1 | 1.6 |

TABLE 38
Percentage of treated cells with residual endogenous TCR or
residual HLA-ABC expression

| | Residual | Residual | Residual | Residual |
|---|---|---|---|---|
| | CD3% | CD3% | HLA-ABC | HLA-ABC |
| Group | (mean) | (SD) | %(mean) | %(SD) |
| TRAC | 1.2 | 0.2 | N/A | N/A |
| TRAC + B2M | 3.0 | 1.0 | 8.4 | 0.2 |
| B2M | N/A | N/A | 7.1 | 0.4 |
| Non-Treated | 94.5 | 0.5 | 100.0 | 0.1 |

### Example 14. Transcriptome Profiling of Engineered T Cells

Transcriptome profiling was used to directly compare the impact of electroporation (EP) and lipid nanoparticle (LNP) engineering methods on the T cell transcriptome, The NanoString nCounter® CAR-T Characterization Panel (measures eight essential components of T cell biology with 780 human genes). Genes included in the CAR-T Characterization Panel are organized and linked to various advanced analysis modules to allow for efficient exploration of the eight essential aspects of T cell biology including activation, exhaustion, metabolism, phenotype, TCR diversity, toxicity, cell types, and persistence. Cells were edited at the AAVS1 locus because knockout of the AAVS1 locus does not induce alterations of T cell transcriptome.

### Example 14.1. T Cell Preparation

Healthy human donor apheresis was obtained commercially (Hemacare), and cells were washed and re-suspended in CliniMACS® PBS/EDTA buffer (Miltenyi Biotec Cat. No. 130-070-525) on the LOVO device. T cells were isolated via negative selection using EasySep™ Human T Cell Isolation Kit (StemCell Technologies, Cat. No. 17951). T cells were aliquoted into vials and cryopreserved in a 1:1 formulation of Cryostor® CS10 (StemCell Technologies Cat. No. 07930) and Plasmalyte A (Baxter Cat. No. 2B2522X) for future use.

Upon thaw, T cells were plated at a density of 1.0×10\hat{ }6 cells/mL in OpTmizer-based media containing CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15). T cells were activated with TransAct™ (1:100 dilution, Miltenyi Biotec) in this media for 24 hours, at which time they were washed and plated in triplicate for editing.

### Example 14.2. T Cell Editing with Lipid Nanoparticles

LNPs were generally prepared as described in Example 1 at a ratio of 50/10/38.5/1.5 Lipid A, cholesterol, DSPC, and PEG2k-DMG. LNPs were prepared with a ratio of gRNA to mRNA of 1:2 by weight. LNPs containing Cas9 mRNA and a sgRNA G000562 (SEQ ID NO: 710) targeting AAVS1 were formulated as described in Example 1. Each LNP preparation was incubated in OpTmizer-based media with cytokines as described above supplemented with 10 ug/ml recombinant human ApoE3 (Peprotech, Cat. 350-02) for 15 minutes at 37° C. Twenty-four hours post activation, T cells were washed and suspended in OpTmizer media with cytokines as described but without human serum. Pre-incubated LNP mix was added to the each well of 100,000 cells to yield a final concentration of 2.5 ug/ml. A control group including unedited T cells (no LNP) was also included. At 6 hours post-delivery, cell pellets were collected for RNA extraction.

### Example 14.3. RNP Electroporation of T Cells

Electroporation was performed 24 hours post activation. AAVS1 targeting sgRNA G000562 (SEQ ID NO: 710) was denatured for 2 minutes at 95° C. before cooling at room temperature for 10 minutes. RNP mixture of 20 uM sgRNA and 10 uM Cas9-NLS protein (SEQ ID NO. 16) was prepared and incubated at 25° C. for 10 minutes. 12.5 μL of RNP mixture was combined with 10,000,000 cells in 87.5 μL P3 electroporation Buffer (Lonza). 100 μL of RNP/cell mix was transferred to the corresponding cuvette. Cells were electroporated in duplicate with the manufacturer's pulse code EH 115. T cell base media was added to the cells immediately post electroporation. At 6 and 24 hours post-delivery, cell pellets were collected for RNA extraction.

### Example 14.4 Transcriptome Profiling

Messenger RNA isolation was performed with RNeasy Mini Kit (Qiagen, Cat. 74106) and transcript profiling was performed with nCounter Human CAR-T Characterization Panel (NanoString, Cat. XT-CSO-CART1-12) according to the manufacturer's protocols. Briefly, extracted mRNA was diluted to 20 ng/μl. Samples and diluted standards were hybridized with Reporter Codeset and Capture Codeset in a 15 μl reaction volume at 65° C. for at least 16 hours. After hybridization, sample cartridges, prep plates, and other consumables were loaded to the NanoString Prep Station (NanoString, Cat. NCT-PREP-120). The samples were then processed onto the cartridges and scanned with the Digital Analyzer.

The scanned RCC files passed all four Quality Control (QC) checks. Data were analyzed using NanoString nSolver 4.0 software. The Gene expression heatmap was generated in the Basic Analysis module. The statistical significance of differential gene expression and pathway scoring was determined by t test in nSolver 4.0 software.

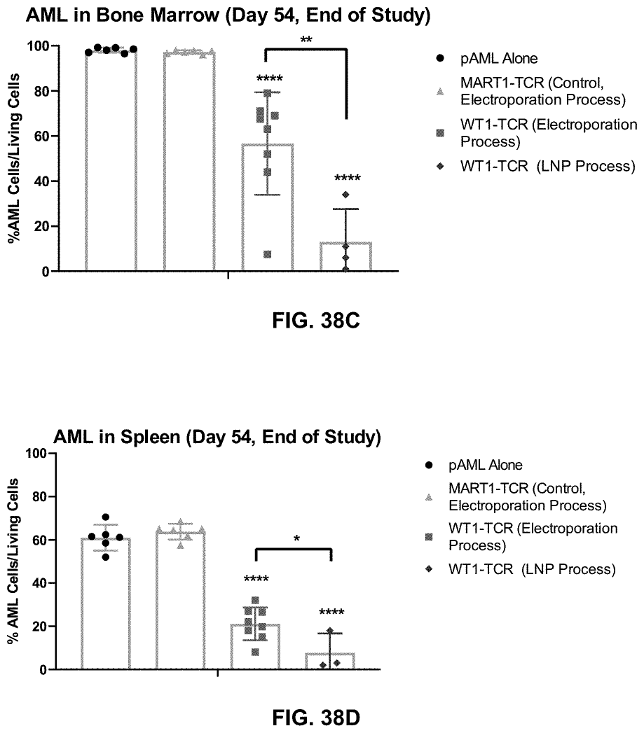

FIG. 37 shows a heat map of transcript expression levels. We found that at 6 hours post-treatment, EP-mediated delivery of RNP significantly (p<0.05) alters T cell expression of a larger gene set, compared to LNP delivery of Cas9 mRNA and gRNA (196 genes vs 75 genes), spanning most T cell-centered cellular pathways represented on this Nanostring array. Perturbations due to LNP delivery are not statistically distinguishable from control delivery (vehicle).

### Example 15. In Vivo Efficacy of Engineered T Cells in AML Model

WT1 specific tgTCR-T cells were engineered using an AAV donor template (see SEQ ID NO: 9) and introducing CRISPR/Cas9 components targeting the genes encoding TCRα and TRBCβ (TRAC and TRBC1/2 respectively) by electroporation of Cas9/sgRNA ribonucleoproteins (RNPs) or by transfection with LNPs containing Cas9 mRNA and sgRNAs.

### Example 15.1. T Cell Preparation

Healthy human donor apheresis was obtained commercially (Hemacare), washed and re-suspended in CliniMACS PBS/EDTA buffer on the LOVO device. T cells were isolated via positive selection using CD4 and CD8 magnetic beads using the CliniMACS Plus and CliniMACS LS disposable kit. T cells were aliquoted into vials and cryopreserved in a 1:1 formulation of Cryostor CS10 and Plasmalyte A for future use. Cryopreserved T cells were thawed and rested overnight at a density of 1.5×10\hat{ }6 cells/ml in complete T cell growth media (TCGM, XVIVO-15 media or CTS Optimizer media, supplemented 5% human AB serum, 2 mM L-Glutamine, 1% Penicillin/Streptomycin, 1×2-Mercaptoethanol, IL-2 (200 U/mL), IL7 (5 ng/mL), IL-15 (5 ng/mL). The following day, T cells were activated with T cell TransAct Reagent (1:100 dilution) for 48 hours prior to editing.

### Example 15.2. T Cell Editing with Ribonucleoprotein Electroporation

RNPs were formed at a 20 μM stock concentration by mixing Cas9-NLS protein (SEQ ID NO: 16) with heat denatured sgRNAs targeting either TRAC (G013006) (SEQ ID NO: 708) or TRBC (G016239) (SEQ ID NO: 707) at a 2:1 guide:Cas9 ratio by weight for 15 minutes. Forty-eight hours post activation, T cells were harvested, centrifuged, and resuspended at a concentration of 20×10\hat{ }6 T cells/100 μL in P3 electroporation buffer (Lonza). The cell suspension was mixed with RNPs to achieve a final RNP concentration of 2 before being transferred to a Nucleofector Cuvette and electroporated. Electroporated T cells were immediately rested in 400 μL TCGM without cytokines for 10 minutes. Cells were plated at a density of 5×10\hat{ }6 cells/well/5 mL in complete TCGM media with AAV6 with a homology directed repair template encoding a WT1 TCR (SEQ ID NO. 9) or MART 1 specific TCR (Journal of Immunology. 2006. 177 (9) 6548-6559) at a MOI of 3×10\hat{ }5 vg/cell. After 24 hours T cells were harvested, washed, and added to a GRex® cell culture system (Wilson Wolf) in complete TCGM media. T cells were cultured for 9-days, with media exchanges every other day, before being evaluated for expansion, tgTCR insertion and endogenous TCR knockout by flow cytometry. T cells were subsequently cryopreserved in CryoStor® CS10 media.

### Example 15.3. T Cell Editing by Lipid Nanoparticle

The T cells engineered in Example 4 by the LNP process were used in this experiment.

### Example 15.4. Flow Cytometry

Engineered T cells were incubated in a cocktail of antibodies targeting CD3, CD4, CD8, along with an anti-Vβ8 antibody (which binds to the TRBC used by the WT1 tgTCR) or a MART1 tetramer in FACS Buffer (PBS pH 7.4, 2% FBS, 1 mM EDTA). T cells were subsequently washed and analyzed on a Cytoflex instrument (Beckman Coulter). Data analysis was performed using FlowJo software package (v.10.6.1). T cells were gated on size, CD4 or CD8 expression, and analyzed for WT1 tgTCR (Vβ8+CD3+) or MART1 tgTCR (MART1 tetramer+ & CD3+).

### Example 15.5 Engineered T Cell Efficacy in AML In Vivo Model

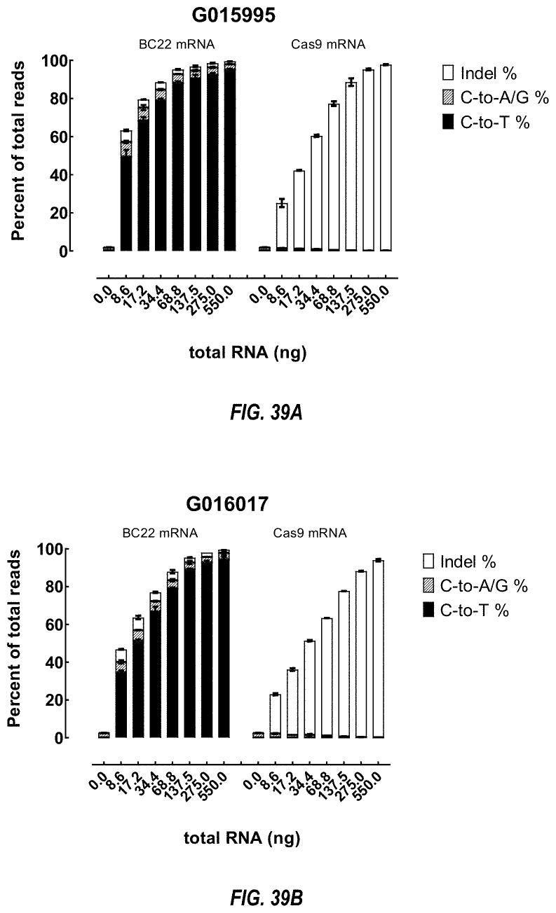

To evaluate the efficacy and specificity of WT1-TCR T cells made by EP and LNP processes, primary leukemic blasts harvested from an HLA-A*02:01+ patient were infused into immunodeficient mice. The mice were treated with the EP or LNP-engineered T cells, and leukemia growth was monitored. FIG. 38A shows a timeline of an in vivo experiment for mice treated with engineered WT1 T cells and controls. FIG. 38B shows AML leukemic blasts outgrowth measured over time as cells per microliter of blood upon treatment of the four groups of mice of FIG. 38A. Briefly, mice treated with engineered WT1-TCR T cells made by EP and LNP processes, T cells transduced with an unrelated MART1-TCR, or another control without any treatment (leukemic blasts only) were compared. Leukemic blasts outgrowth in bone marrow (FIG. 38C) and in spleen (FIG. 38D) was measured as percentage of AML cells per total live cells upon treatment of mice as in FIG. 38A.

### Example 16A. LNP Titration in T Cells with Fixed Ratio of BC22n:UGI

Using LNP delivery to activated human T cells, the potency of single-target and multi-target editing was assessed with either Cas9 or a deaminase (BC22n).

### Example 16.A.1. T Cell Preparation

Healthy human donor apheresis was obtained commercially (Hemacare), and cells were washed and re-suspended in CliniMACS® PBS/EDTA buffer (Miltenyi Biotec Cat. No. 130-070-525) on the LOVO device. T cells were isolated via positive selection using CD4 and CD8 magnetic beads (Miltenyi Biotec Cat. No. 130-030-401/130-030-801) using the CliniMACS® Plus and CliniMACS® LS disposable kit. T cells were aliquoted into vials and cryopreserved in a 1:1 formulation of Cryostor® CS10 (StemCell Technologies Cat. No. 07930) and Plasmalyte A (Baxter Cat. No. 2B2522X) for future use. Upon thaw, T cells were plated at a density of 1.0×10e6 cells/mL in T cell basal media composed of X-VIVO 15™ serum-free hematopoietic cell medium (Lonza Bioscience) containing 5% (v/v) of fetal bovine serum, 50 μM of 2-Mercaptoethanol, 10 mM of N-Acetyl-L-(+)-cysteine, 10 U/mL of Penicillin-Streptomycin, in addition to 1× cytokines (200 U/mL of recombinant human interleukin-2, 5 ng/mL of recombinant human interleukin-7 and 5 ng/mL of recombinant human interleukin-15). T cells were activated with TransAct™ (1:100 dilution, Miltenyi Biotec). Cells were expanded in T cell basal media for 72 hours prior to LNP transfection.

### Example 16.A.2. T Cell Editing

Each RNA species, i.e., UGI mRNA, Guide RNA or editor mRNA, was formulated separately in an LNP as described in Example 1. Editor mRNAs encoded either BC22n (SEQ ID NO: 18) or Cas9. Guides targeting B2M (G015995) (SEQ ID NO: 711), TRAC (G016017) (SEQ ID NO: 712), TRBC1/2 (G016206) (SEQ ID NO: 713), and CIITA (G018117) (SEQ ID NO: 714) were used either singly or in combination. Messenger RNA encoding UGI (SEQ ID NO: 21) was delivered in both Cas9 and BC22n arms of the experiment to normalize lipid amounts. Previous experiments have established UGI mRNA does not impact total editing or editing profile when used with Cas9 mRNA. LNPs were mixed to fixed total mRNA weight ratios of 6:3:2 for editor mRNA, guide RNA, and UGI mRNA respectively as described in Table 12. In the 4-guide experiment described in Table 39, individual guides were diluted 4-fold to maintain the overall 6:3 editor mRNA: guide weight ratio and to allow comparison to individual guide potency based on total lipid delivery. LNP mixtures were incubated for 5 minutes at 37° C. in T cell basal media substituting 6% cynomolgus monkey serum (Bioreclamation IVT, Cat. CYN220760) for fetal bovine serum.

Seventy-two hours post activation, T cells were washed and suspended in basal T cell media. Pre-incubated LNP mix was added to the each well with 1×10e5 Tcells/well. T cells were incubated at 37° C. with 5% CO2 for the duration of the experiment. T cell media was changed 6 days and 8 days after activation and on tenth day post activation, cells were harvested for analysis by NGS and flow cytometry. NGS was performed as in Example 1.4.

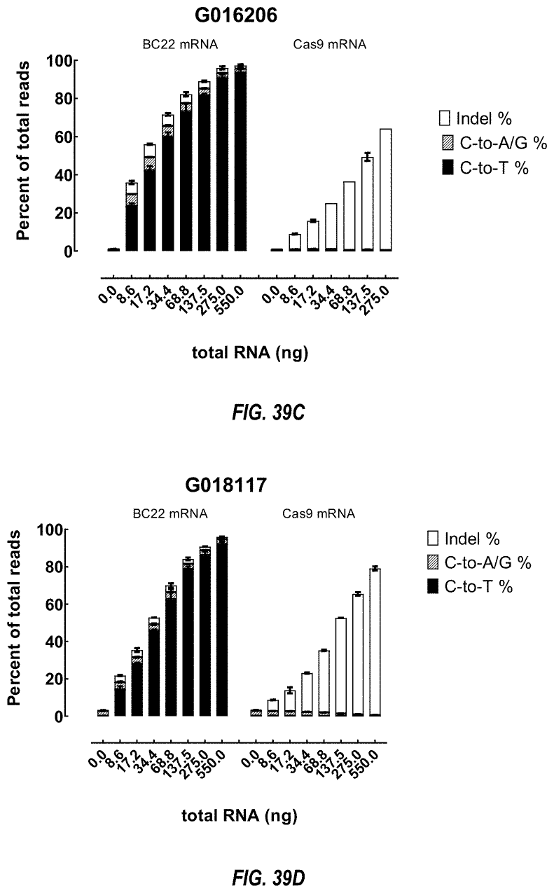

Table 39 and FIGS. 39A-D describe the editing profile of T cells when an individual guide was used for editing. Total editing and C to T editing showed direct, dose responsive relationships to increasing amounts of BC22n mRNA, UGI mRNA and guide across all guides tested. Indel and C conversions to A or G are in an inverse relationship with dose where lower doses resulted in a higher percentage of these mutations. In samples edited with Cas9, total editing and indel activity increase with the total RNA dose.

TABLE 39
Editing as a percent of total reads—single guide delivery.

| | | Total | C-to- | | C-to- | | | | |
|---|---|---|---|---|---|---|---|---|---|
| | | (ng) | T % | | A/G % | | Indel % | | |
| Guide | Editor | RNA | mean | SD | mean | SD | mean | SD | N |
| G015995 | BC22n | 0.0 | 0.3 | 0.0 | 1.5 | 0.1 | 0.2 | 0.0 | 2 |
| B2M | | 8.6 | 49.5 | 3.5 | 7.7 | 0.6 | 6.0 | 0.4 | 2 |
| | | 17.2 | 68.5 | 1.7 | 6.7 | 1.3 | 4.3 | 0.1 | 2 |
| | | 34.4 | 79.0 | 0.9 | 5.7 | 0.3 | 3.8 | 0.0 | 2 |
| | | 68.8 | 88.2 | 0.8 | 4.6 | 0.0 | 2.5 | 0.2 | 2 |
| | | 137.5 | 90.6 | 1.8 | 4.1 | 0.4 | 2.2 | 0.5 | 2 |
| | | 275.0 | 92.6 | 0.8 | 3.7 | 0.3 | 2.2 | 0.3 | 2 |
| | | 550.0 | 95.2 | 0.4 | 2.8 | 0.0 | 1.6 | 0.2 | 2 |
| | Cas9 | 0.0 | 0.3 | 0.0 | 1.5 | 0.2 | 0.2 | 0.0 | 2 |
| | | 8.6 | 0.3 | 0.0 | 1.2 | 0.1 | 23.7 | 2.1 | 2 |
| | | 17.2 | 0.3 | 0.0 | 0.9 | 0.1 | 41.1 | 0.2 | 2 |
| | | 34.4 | 0.3 | 0.0 | 0.6 | 0.0 | 59.4 | 0.6 | 2 |
| | | 68.8 | 0.2 | 0.1 | 0.4 | 0.0 | 76.8 | 1.2 | 2 |
| | | 137.5 | 0.1 | 0.1 | 0.2 | 0.0 | 88.2 | 2.0 | 2 |
| | | 275.0 | 0.1 | 0.0 | 0.1 | 0.1 | 95.1 | 0.5 | 2 |
| | | 550.0 | 0.1 | 0.0 | 0.1 | 0.0 | 97.5 | 0.3 | 2 |
| G016017 | BC22n | 0.0 | 0.2 | 0.0 | 2.2 | 0.1 | 0.2 | 0.1 | 2 |
| TRAC | | 8.6 | 34.6 | 1.1 | 5.6 | 0.8 | 6.6 | 0.2 | 2 |
| | | 17.2 | 51.3 | 0.8 | 5.7 | 0.1 | 6.7 | 1.0 | 2 |
| | | 34.4 | 66.9 | 2.6 | 5.4 | 0.2 | 4.7 | 0.4 | 2 |
| | | 68.8 | 79.0 | 0.6 | 4.4 | 0.7 | 4.5 | 0.9 | 2 |
| | | 137.5 | 89.2 | 0.4 | 3.6 | 0.9 | 2.5 | 0.2 | 2 |
| | | 275.0 | 92.8 | 0.9 | 2.9 | 0.0 | 2.3 | 0.0 | 2 |
| | | 550.0 | 94.5 | 1.3 | 3.4 | 1.0 | 1.6 | 0.2 | 2 |
| | Cas9 | 0.0 | 0.2 | 0.0 | 2.3 | 0.1 | 0.1 | 0.0 | 2 |
| | | 8.6 | 0.2 | 0.0 | 2.1 | 0.2 | 20.7 | 0.5 | 2 |
| | | 17.2 | 0.1 | 0.0 | 1.4 | 0.0 | 34.6 | 0.7 | 2 |
| | | 34.4 | 0.1 | 0.0 | 1.5 | 0.4 | 49.8 | 0.4 | 2 |
| | | 68.8 | 0.1 | 0.0 | 1.0 | 0.0 | 62.3 | 0.1 | 2 |
| | | 137.5 | 0.1 | 0.0 | 0.6 | 0.1 | 77.0 | 0.1 | 2 |
| | | 275.0 | 0.0 | 0.0 | 0.3 | 0.0 | 87.8 | 0.2 | 2 |
| | | 550.0 | 0.0 | 0.0 | 0.2 | 0.0 | 93.8 | 0.6 | 2 |
| G016206 | BC22n | 0.0 | 0.4 | 0.1 | 0.6 | 0.1 | 0.1 | 0.1 | 2 |
| TRBC1/2 | | 8.6 | 23.7 | 1.3 | 6.1 | 0.0 | 6.1 | 0.8 | 2 |
| | | 17.2 | 42.4 | 2.2 | 6.8 | 0.1 | 6.8 | 0.3 | 2 |
| | | 34.4 | 60.1 | 2.2 | 5.7 | 0.3 | 5.9 | 0.7 | 2 |
| | | 68.8 | 73.2 | 4.2 | 4.3 | 0.1 | 4.7 | 1.1 | 2 |
| | | 137.5 | 81.7 | 0.8 | 3.6 | 0.2 | 3.7 | 0.4 | 2 |
| | | 275.0 | 91.0 | 1.7 | 2.3 | 0.1 | 2.8 | 0.8 | 2 |
| | | 550.0 | 93.6 | 1.9 | 2.0 | 0.2 | 1.7 | 0.6 | 2 |
| | Cas9 | 0.0 | 0.3 | 0.0 | 0.5 | 0.0 | 0.1 | 0.0 | 1 |
| | | 8.6 | 0.3 | 0.2 | 0.5 | 0.1 | 8.1 | 0.2 | 2 |
| | | 17.2 | 0.3 | 0.1 | 0.7 | 0.1 | 14.9 | 0.6 | 2 |
| | | 34.4 | 0.2 | 0.0 | 0.8 | 0.0 | 24.1 | 0.0 | 1 |
| | | 68.8 | 0.2 | 0.0 | 0.4 | 0.0 | 35.9 | 0.0 | 1 |
| | | 137.5 | 0.2 | 0.0 | 0.5 | 0.0 | 48.6 | 2.1 | 2 |
| | | 275.0 | 0.1 | 0.0 | 0.4 | 0.0 | 63.8 | 0.0 | 1 |
| | | 550.0 | Not assayed | | | | | | |
| G018117 | BC22n | 0.0 | 0.3 | 0.0 | 2.7 | 0.1 | 0.3 | 0.0 | 2 |
| CIITA | | 8.6 | 14.5 | 1.5 | 3.8 | 0.3 | 3.5 | 0.3 | 2 |
| | | 17.2 | 28.1 | 0.6 | 3.5 | 0.3 | 3.9 | 1.0 | 2 |
| | | 34.4 | 45.9 | 0.4 | 3.3 | 0.4 | 3.6 | 0.0 | 2 |
| | | 68.8 | 62.8 | 5.3 | 3.6 | 0.1 | 3.7 | 1.2 | 2 |
| | | 137.5 | 78.9 | 1.3 | 2.7 | 0.1 | 2.7 | 0.7 | 2 |
| | | 275.0 | 86.3 | 1.8 | 2.6 | 0.1 | 2.0 | 0.1 | 2 |
| | | 550.0 | 92.3 | 1.2 | 2.6 | 0.2 | 1.1 | 0.2 | 2 |
| | Cas9 | 0.0 | 0.2 | 0.0 | 2.8 | 0.1 | 0.3 | 0.0 | 2 |
| | | 8.6 | 0.3 | 0.0 | 2.5 | 0.0 | 6.0 | 0.2 | 2 |
| | | 17.2 | 0.2 | 0.0 | 2.4 | 0.1 | 11.2 | 1.6 | 2 |
| | | 34.4 | 0.2 | 0.0 | 2.1 | 0.0 | 20.8 | 0.3 | 2 |
| | | 68.8 | 0.2 | 0.0 | 1.9 | 0.1 | 33.2 | 0.4 | 2 |
| | | 137.5 | 0.1 | 0.0 | 1.3 | 0.1 | 51.2 | 0.0 | 2 |
| | | 275.0 | 0.1 | 0.0 | 0.9 | 0.2 | 64.5 | 0.9 | 2 |
| | | 550.0 | 0.1 | 0.0 | 0.6 | 0.0 | 78.4 | 1.1 | 2 |

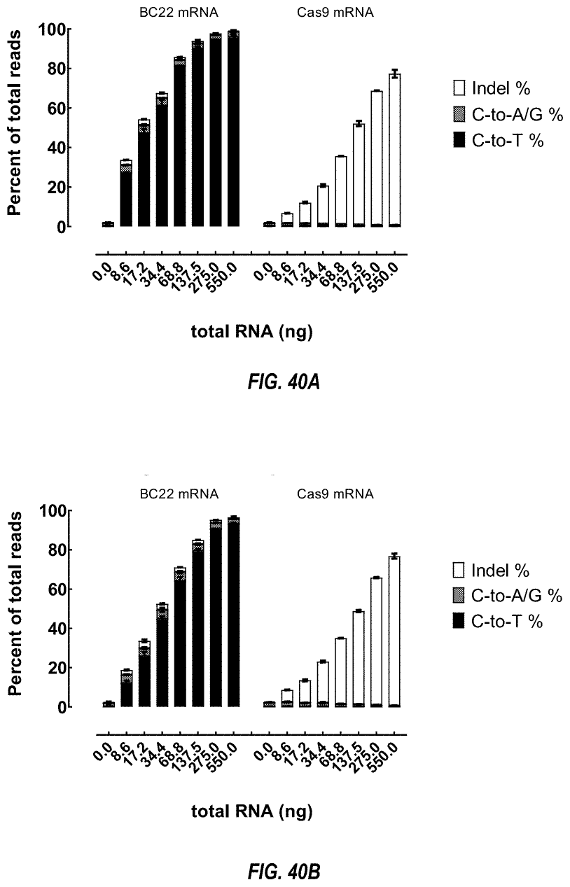

Table 40 and FIGS. 40A-D describe the editing profile for T cells in percent of total reads when four guides were used simultaneously for editing. In this arm of the experiment, each guide was used at 25% the concentration compared to the single guide editing experiment. In total, T cells were exposed to 6 different LNPs simultaneously (editor mRNA, UGI mRNA, 4 guides). Editing with BC22n and trans UGI lead to higher percentages of maximum total editing for each locus compared to editing with Cas9.

TABLE 40
Editing as a percentage of
total reads—multiple guide delivery.

| | | Total | C-to- | | C-to- | | | | |
|---|---|---|---|---|---|---|---|---|---|
| Locus | | RNA | T % | | A/G % | | Indel % | | |
| Assayed | Editor | (ng) | mean | SD | mean | SD | mean | SD | N |
| G015995 | BC22n | 0.0 | 0.3 | 0.0 | 1.5 | 0.2 | 0.2 | 0.0 | 2 |
| B2M | | 8.6 | 27.3 | 0.2 | 3.8 | 0.1 | 2.6 | 0.1 | 2 |
| | | 17.2 | 47.2 | 2.2 | 4.1 | 0.4 | 3.0 | 0.1 | 2 |
| | | 34.4 | 61.2 | 3.0 | 3.9 | 0.1 | 2.6 | 0.3 | 2 |
| | | 68.8 | 81.4 | 0.1 | 2.9 | 0.1 | 1.4 | 0.1 | 2 |
| | | 137.5 | 90.0 | 1.1 | 2.6 | 0.3 | 1.3 | 0.5 | 2 |
| | | 275.0 | 94.7 | 0.1 | 2.2 | 0.1 | 0.8 | 0.0 | 2 |
| | | 550.0 | 95.9 | 0.9 | 2.9 | 1.0 | 0.4 | 0.3 | 2 |
| | Cas9 | 0.0 | 0.3 | 0.0 | 1.4 | 0.1 | 0.2 | 0.0 | 2 |
| | | 8.6 | 0.3 | 0.0 | 1.4 | 0.0 | 5.0 | 0.1 | 2 |
| | | 17.2 | 0.3 | 0.0 | 1.3 | 0.0 | 10.5 | 0.4 | 2 |
| | | 34.4 | 0.3 | 0.0 | 1.1 | 0.0 | 19.3 | 0.6 | 2 |
| | | 68.8 | 0.3 | 0.0 | 0.9 | 0.0 | 34.4 | 0.1 | 2 |
| | | 137.5 | 0.2 | 0.0 | 0.7 | 0.0 | 51.1 | 1.3 | 2 |
| | | 275.0 | 0.2 | 0.1 | 0.5 | 0.0 | 68.0 | 0.1 | 2 |
| | | 550.0 | 0.3 | 0.1 | 0.4 | 0.1 | 76.7 | 2.0 | 2 |
| G016017 | BC22n | 0.0 | 0.1 | 0.1 | 1.9 | 0.6 | 0.2 | 0.0 | 2 |
| TRAC | | 8.6 | 12.1 | 1.3 | 4.3 | 0.2 | 2.4 | 0.2 | 2 |
| | | 17.2 | 25.7 | 2.2 | 4.2 | 0.5 | 3.8 | 0.7 | 2 |
| | | 34.4 | 44.7 | 1.4 | 4.7 | 1.0 | 3.0 | 0.3 | 2 |
| | | 68.8 | 64.2 | 1.9 | 4.4 | 0.6 | 2.5 | 0.1 | 2 |
| | | 137.5 | 79.3 | 1.1 | 3.6 | 0.4 | 2.1 | 0.1 | 2 |
| | | 275.0 | 90.7 | 0.0 | 3.0 | 0.1 | 1.5 | 0.0 | 2 |
| | | 550.0 | 93.3 | 0.6 | 2.4 | 0.1 | 0.9 | 0.4 | 2 |
| | Cas9 | 0.0 | 0.1 | 0.1 | 2.1 | 0.2 | 0.1 | 0.0 | 2 |
| | | 8.6 | 0.2 | 0.1 | 2.3 | 0.2 | 6.1 | 0.2 | 2 |
| | | 17.2 | 0.1 | 0.0 | 1.8 | 0.2 | 11.5 | 0.5 | 2 |
| | | 34.4 | 0.1 | 0.0 | 2.0 | 0.4 | 21.0 | 0.4 | 2 |
| | | 68.8 | 0.1 | 0.0 | 1.4 | 0.0 | 33.5 | 0.1 | 2 |
| | | 137.5 | 0.1 | 0.0 | 1.2 | 0.1 | 47.5 | 0.5 | 2 |
| | | 275.0 | 0.1 | 0.0 | 0.9 | 0.1 | 64.8 | 0.2 | 2 |
| | | 550.0 | 0.1 | 0.0 | 0.6 | 0.1 | 76.1 | 1.3 | 2 |
| G016206 | BC22n | 0.0 | No data | | | | | | |
| TRBC1/2 | | 8.6 | 11.6 | 0.3 | 2.6 | 0.2 | 2.8 | 0.3 | 2 |
| | | 17.2 | 23.4 | 0.4 | 3.6 | 0.3 | 2.6 | 0.5 | 2 |
| | | 34.4 | 38.5 | 1.4 | 3.7 | 0.2 | 2.9 | 0.7 | 2 |
| | | 68.8 | 55.6 | 1.7 | 2.3 | 0.4 | 2.4 | 0.0 | 2 |
| | | 137.5 | 72.4 | 1.2 | 1.8 | 0.5 | 1.7 | 0.5 | 2 |
| | | 275.0 | 85.1 | 1.0 | 1.9 | 0.5 | 1.7 | 0.6 | 2 |
| | | 550.0 | 89.8 | 2.8 | 2.2 | 0.1 | 0.9 | 0.3 | 2 |
| | Cas9 | 0.0 | 0.2 | 0.0 | 0.6 | 0.0 | 0.1 | 0.0 | 1 |
| | | 8.6 | 0.2 | 0.1 | 0.7 | 0.1 | 2.3 | 0.3 | 2 |
| | | 17.2 | 0.3 | 0.0 | 0.7 | 0.3 | 4.2 | 0.4 | 2 |
| | | 34.4 | 0.1 | 0.0 | 0.5 | 0.1 | 6.6 | 0.5 | 2 |
| | | 68.8 | 0.4 | 0.0 | 0.5 | 0.0 | 12.3 | 0.0 | 1 |
| | | 137.5 | 0.2 | 0.0 | 0.5 | 0.0 | 17.8 | 0.0 | 1 |
| | | 275.0 | 0.1 | 0.0 | 0.5 | 0.0 | 33.0 | 0.0 | 1 |
| | | 550.0 | 0.3 | 0.2 | 0.3 | 0.0 | 43.3 | 1.7 | 2 |
| G018117 | BC22n | 0.0 | 0.2 | 0.0 | 2.6 | 0.1 | 0.3 | 0.0 | 2 |
| CIITA | | 8.6 | 4.6 | 0.9 | 3.1 | 0.2 | 0.8 | 0.2 | 2 |
| | | 17.2 | 10.5 | 0.2 | 2.9 | 0.1 | 1.1 | 0.2 | 2 |
| | | 34.4 | 18.8 | 0.3 | 2.9 | 0.2 | 1.6 | 0.2 | 2 |
| | | 68.8 | 35.1 | 0.6 | 2.7 | 0.2 | 1.6 | 0.7 | 2 |
| | | 137.5 | 52.9 | 0.2 | 2.9 | 0.3 | 1.5 | 0.0 | 2 |
| | | 275.0 | 71.9 | 2.4 | 2.5 | 0.3 | 1.3 | 0.1 | 2 |
| | | 550.0 | 81.1 | 1.9 | 2.6 | 0.1 | 1.1 | 0.6 | 2 |
| | Cas9 | 0.0 | 0.3 | 0.0 | 2.7 | 0.1 | 0.3 | 0.0 | 2 |
| | | 8.6 | 0.2 | 0.0 | 2.6 | 0.2 | 1.4 | 0.0 | 2 |
| | | 17.2 | 0.2 | 0.0 | 2.5 | 0.0 | 2.1 | 0.3 | 2 |
| | | 34.4 | 0.3 | 0.0 | 2.5 | 0.0 | 3.9 | 0.1 | 2 |
| | | 68.8 | 0.2 | 0.0 | 2.5 | 0.2 | 7.7 | 0.6 | 2 |
| | | 137.5 | 0.2 | 0.0 | 2.2 | 0.1 | 13.3 | 0.2 | 2 |
| | | 275.0 | 0.1 | 0.0 | 1.9 | 0.0 | 26.7 | 1.3 | 2 |
| | | 550.0 | 0.1 | 0.0 | 1.7 | 0.1 | 42.3 | 0.3 | 2 |

On day 10 post-activation, T cells were phenotyped by flow cytometry to determine if editing resulted in loss of cell surface proteins. Briefly, T cells were incubated in a mix of the following antibodies: B2M-FITC (BioLegend, Cat. 316304), CD3-AF700 (BioLegend, Cat. 317322), HLA DR DQ DP-PE (BioLegend, Cat 361704) and DAPI (BioLegend, Cat 422801). A subset of unedited cells was incubated with Isotype Control-PE (BioLegend® Cat. No. 400234). Cells were subsequently washed, processed on a Cytoflex instrument (Beckman Coulter) and analyzed using the FlowJo software package. T cells were gated based on size, shape, viability, and antigen expression.

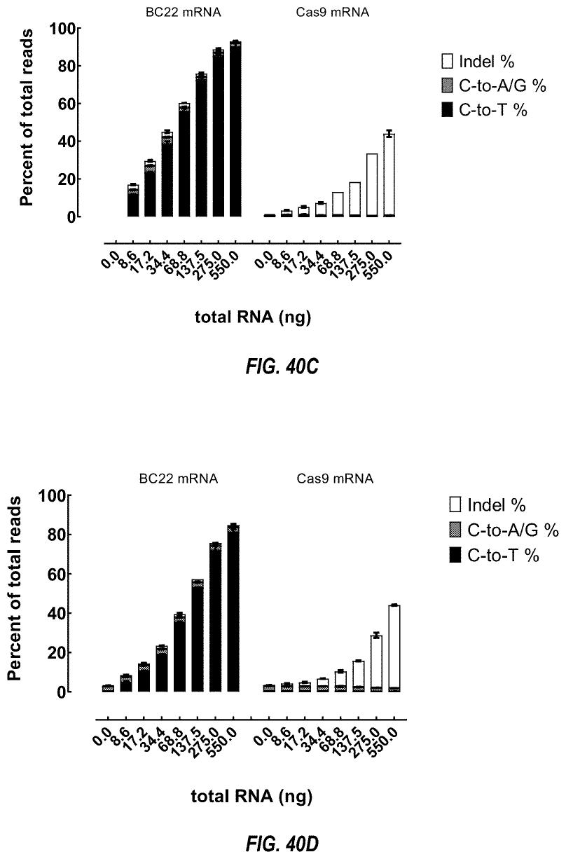

Table 41 and FIGS. 41A-H report phenotyping results as percent of cells negative for antibody binding. The percentage of antigen negative cells increased in a dose responsive manner with increasing total RNA for both BC22n and Cas9 samples. Cells edited with BC22n showed comparable or higher protein knockout compared to cells edited with Cas9 for all guides tested. In multi-edited cells, BC22n with trans UGI showed substantially higher percentages of antigen negative cells than Cas9 with trans UGI. For example, BC22 edited samples at the highest total RNA dose of 550 ng showed 84.2% of cells lacking all three antigens, while Cas9 editing led to only 46.8% such triple knockout cells. For samples treated with one guide only, the correlation between DNA editing and antigen reduction was robust. BC22n had an R square measurement of 0.93 when comparing C to T conversions to antigen knockout. Cas9 had an R square measurement of 0.95 when comparing indels to antigen knockout.

TABLE 41
Flow cytometry data—percent
cells negative for antigen (n = 2).

| | | Total | BC22n | | Cas9 | |
|---|---|---|---|---|---|---|
| | Pheno- | RNA | Mean | | Mean | |
| Guide(s) | type | (ng) | % | SD | % | SD |
| G015995 | B2M | 550.0 | 95.7 | 0.1 | 91.3 | 0.6 |
| B2M | neg | 275.0 | 94.4 | 0.4 | 89.3 | 0.1 |
| | | 137.5 | 91.2 | 0.1 | 82.1 | 3.3 |
| | | 68.8 | 83.9 | 0.4 | 68.7 | 3.3 |
| | | 34.4 | 75.7 | 1.4 | 53.4 | 0.2 |
| | | 17.2 | 60.8 | 2.0 | 30.7 | 1.3 |
| | | 8.6 | 44.0 | 2.3 | 13.9 | 2.0 |
| | | 0.0 | 14.1 | 4.1 | 9.9 | 1.9 |
| G015995 | B2M | 550.0 | 94.4 | 0.1 | 74.2 | 0.4 |
| G016017 | neg | 275.0 | 91.3 | 0.1 | 65.2 | 0.1 |
| G016206 | | 137.5 | 84.3 | 0.2 | 45.4 | 1.9 |
| G018117 | | 68.8 | 72.7 | 0.4 | 24.5 | 0.8 |
| | | 34.4 | 56.2 | 1.2 | 14.1 | 2.3 |
| | | 17.2 | 38.5 | 0.2 | 9.9 | 0.8 |
| | | 8.6 | 20.6 | 0.7 | 7.6 | 2.4 |
| | | 0.0 | 14.1 | 4.1 | 9.9 | 1.9 |
| G016017 | CD3 | 550.0 | 97.3 | 0.3 | 94.8 | 0.4 |
| TRAC | neg | 275.0 | 96.0 | 0.2 | 87.0 | 4.9 |
| | | 137.5 | 91.9 | 0.2 | 72.7 | 0.9 |
| | | 68.8 | 85.7 | 0.5 | 65.6 | 0.1 |
| | | 34.4 | 76.6 | 0.8 | 51.7 | 3.0 |
| | | 17.2 | 61.8 | 1.7 | 35.7 | 1.1 |
| | | 8.6 | 42.1 | 0.7 | 20.1 | 1.5 |
| | | 0.0 | 1.0 | 0.1 | 0.9 | 0.1 |
| G016206 | CD3 | 550.0 | 97.9 | 0.1 | 86.6 | 0.3 |
| TRBC1/2 | neg | 275.0 | 96.0 | 0.1 | 77.3 | 0.1 |
| | | 137.5 | 90.4 | 0.8 | 59.4 | 0.4 |
| | | 68.8 | 82.9 | 0.1 | 40.6 | 1.2 |
| | | 34.4 | 71.9 | 1.5 | 27.0 | 1.6 |
| | | 17.2 | 53.4 | 0.3 | 16.1 | 0.1 |
| | | 8.6 | 32.6 | 0.6 | 7.9 | 0.4 |
| | | 0.0 | 0.8 | 0.0 | 0.9 | 0.4 |
| G015995 | CD3 | 550.0 | 98.3 | 0.2 | 84.2 | 0.1 |
| G016017 | neg | 275.0 | 96.3 | 0.1 | 74.6 | 0.5 |
| G016206 | | 137.5 | 90.4 | 0.3 | 57.4 | 1.0 |
| G018117 | | 68.8 | 81.3 | 0.3 | 39.4 | 0.1 |
| | | 34.4 | 66.3 | 1.6 | 25.6 | 0.8 |
| | | 17.2 | 48.2 | 1.0 | 15.3 | 0.5 |
| | | 8.6 | 27.3 | 0.7 | 8.6 | 0.5 |
| | | 0.0 | 0.9 | 0.1 | 0.9 | 0.2 |
| G018117 | HLA DR | 550.0 | 95.7 | 0.4 | 72.0 | 0.1 |
| CIITA | DP DQ | 275.0 | 92.5 | 1.1 | 65.6 | 0.4 |
| | neg | 137.5 | 85.2 | 0.6 | 55.5 | 0.6 |
| | | 68.8 | 74.5 | 1.1 | 48.9 | 0.0 |
| | | 34.4 | 65.8 | 3.7 | 40.7 | 0.6 |
| | | 17.2 | 49.9 | 0.1 | 36.2 | 0.6 |
| | | 8.6 | 41.6 | 0.8 | 34.2 | 1.3 |
| | | 0.0 | 30.1 | 1.6 | 35.2 | 0.4 |
| G015995 | HLA DR | 550.0 | 88.0 | 0.2 | 52.8 | 1.1 |
| G016017 | DP DQ | 275.0 | 81.2 | 0.2 | 46.4 | 0.4 |
| G016206 | neg | 137.5 | 70.4 | 1.3 | 39.9 | 1.8 |
| G018117 | | 68.8 | 60.0 | 0.4 | 39.1 | 3.3 |
| | | 34.4 | 48.8 | 0.6 | 37.7 | 2.9 |
| | | 17.2 | 43.0 | 4.2 | 37.5 | 0.6 |
| | | 8.6 | 37.8 | 2.1 | 35.0 | 0.0 |
| | | 0.0 | 33.0 | 1.9 | 37.3 | 2.1 |
| G015995 | B2M neg | 550.0 | 84.2 | 0.0 | 46.8 | 1.1 |
| G016017 | CD3 neg | 275.0 | 76.2 | 0.0 | 37.8 | 0.2 |
| G016206 | HLA DR | 137.5 | 63.0 | 1.3 | 23.4 | 2.4 |
| G018117 | DP DQ | 68.8 | 48.2 | 0.2 | 10.8 | 0.9 |
| | neg | 34.4 | 31.5 | 1.1 | 3.6 | 0.9 |
| | | 17.2 | 17.8 | 1.7 | 1.1 | 0.2 |
| | | 8.6 | 6.4 | 0.0 | 0.4 | 0.1 |
| | | 0.0 | 0.1 | 0.0 | 0.1 | 0.0 |

### Example 16.B. Simultaneous Quadruple Edits with BC22n or Cas9 in T Cells after Delivery Via Electroporation or LNP

To assess the amount of structural genomic changes associated with delivery conditions and editing by Cas9 or base editor, T cells treated with electroporation to deliver RNP or lipid nanoparticles (LNP) to deliver four guides and either Cas9 or BC22n were analyzed for cell viability, DNA double-stranded breaks, editing, surface protein expression, and chromosomal structural.

### Example 16.B.1. T Cell Preparation

Healthy human donor apheresis was obtained commercially (Hemacare), and cells were washed and re-suspended in CliniMACS® PBS/EDTA buffer (Miltenyi Biotec Cat. No. 130-070-525) on the LOVO device. T cells were isolated via negative selection using EasySep™ Human T Cell Isolation Kit (StemCell Technologies Cat. No. 17951). T cells were aliquoted into vials and cryopreserved in a 1:1 formulation of Cryostor® CS10 (StemCell Technologies Cat. No. 07930) and Plasmalyte A (Baxter Cat. No. 2B2522X) for future use.

Upon thaw, T cells were plated at a density of 1.0×10\hat{ }6 cells/mL in OpTmizer-based media containing CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15). T cells were activated with TransAct™ (1:100 dilution, Miltenyi Biotec) in this media for 72 hours, at which time they were washed and plated in quadruplicate for editing either by electroporation or lipid nanoparticle.

### Example 16.B.2. Single gRNA and 4 gRNA T Cell Editing with Lipid Nanoparticles

LNPs were formulated generally as in Example 1 with a single RNA species cargo. Cargo was selected from an mRNA encoding BC22n, an mRNA encoding Cas9, an mRNA encoding UGI, sgRNA G015995 (SEQ ID NO: 711) targeting B2M, sgRNA G016017 (SEQ ID NO: 712) targeting TRAC, sgRNA G016200 targeting TRBC or sgRNA G016086 targeting CIITA. Each LNP was incubated in OpTmizer-based media with cytokines as described above supplemented with 20 ug/ml recombinant human ApoE3 (Peprotech, Cat. 350-02) for 15 minutes at 37° C. Seventy-two hours post activation, T cells were washed and suspended in OpTmizer media with cytokines without human serum. For single sgRNA editing conditions, pre-incubated LNP mix was added to the each well of 100,000 cells to yield a final concentration of 2.3 μg/mL editor mRNA (BC22n or Cas9), 1.1 μg/mL UGI and 4.6 μg/μL G016017 (SEQ ID NO: 712). For four-plex sgRNA editing LNP mix was added to the each well of 100,000 cells to yield a final concentration of 2.3 μg/mL editor mRNA (BC22n or Cas9), 1.1 μg/mL UGI, 1.15 μg/μL G015995 (SEQ ID NO: 711), 1.15 μg/μL G016017 (SEQ ID NO: 712), 1.15 μg/μL G016200 and 1.15 μg/μL G016086. A control group including unedited T cells (no LNP) was also included. At 16 hours post-delivery, a subset of cells was used to measure cell viability and another subset of cells was processed for imaging of γH2AX foci. The remaining T cells continued to expand in culture. Media was changed 5 days and 8 days after activation and on the eleventh day post activation, cells were harvested for analysis by NGS, flow cytometry and UnIT. NGS was performed as in Example 1.

### Example 16.B.3. Single gRNA and 4 gRNA T Cell Editing with mRNA Electroporation

Electroporation was performed 72 hours post activation. sgRNA G015995 (SEQ ID NO: 711) targeting B2M, sgRNA G016017 (SEQ ID NO: 712) targeting TRAC, sgRNA G016200 (SEQ ID NO: 718) targeting TRBC and sgRNA G016086 (SEQ ID NO: 719) were denatured for 2 minutes at 95° C. before cooling at room temperature for 10 minutes. T cells were harvested, centrifuged, and resuspended at a concentration of 12.5×10e6 T cells/mL in P3 electroporation buffer (Lonza). For single sgRNA editing conditions, 1×10e5 T cells were mixed with 40 ng/μL of editor mRNA (BC22n or Cas9), 10 ng/μL of UGI mRNA and 80 pmols of sgRNA in a final volume of 20 μL of P3 electroporation buffer. For four-plex sgRNA editing conditions, 1×10e5 T cells were mixed with 40 ng/μL of editor mRNA (BC22n or Cas9), 10 ng/μL of UGI mRNA and 20 pmols of the four individual sgRNA in a final volume of 20 μL of P3 electroporation buffer. This mix was transferred in quadruplicate to a 96-well Nucleofector™ plate and electroporated using a manufacturer's pulse code. Electroporated T cells were rested in 80 μL of OpTmizer-based media with cytokines before being transferred to a new flat-bottom 96-well plate. A control group including unedited T cells (no EP) was also included. At 16 hours post-delivery, a subset of cells was used to measure cell viability and another subset of cells was processed for imaging of γH2AX foci.

### Example 16.B.4. Relative Viability Via Cell Titer Glo

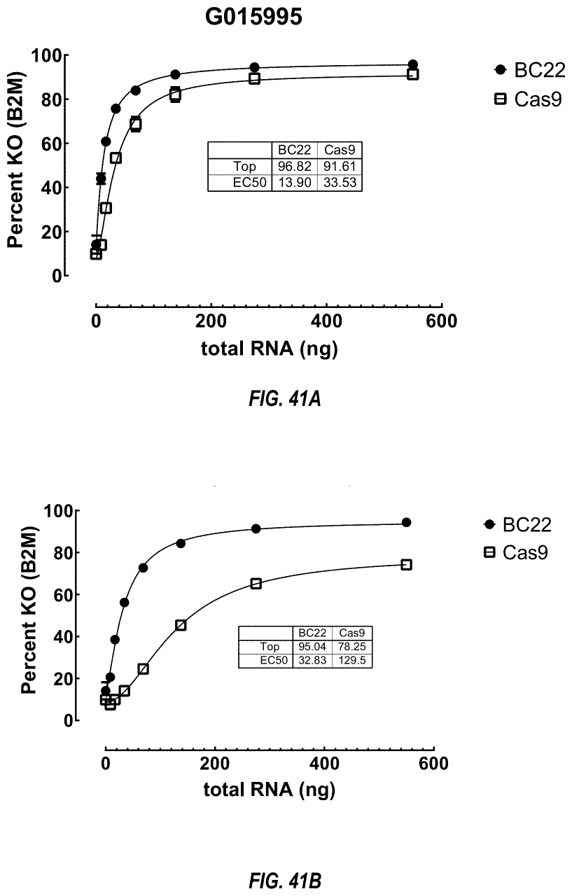

Sixteen hours post electroporation or lipid nanoparticle delivery 20 μL of control or edited cells were removed from original plate and added to a new flat-bottom 96-well plate with black walls (Corning Cat. 3904). CellTiter-Glo® 2.0 (Promega Cat. G9241) was added and samples were processed according to manufacturer's protocol. Relative luminescence units (RLU) were readout by the CLARIstar plus (BMG Labtech) plate reader with gain set at 3600. Relative viability as shown in Table 42 and FIG. 42 was calculated by dividing all sample RLU by the average of untreated control RLU. All electroporation conditions had a greater than 5-fold viability drop from untreated control levels whereas LNP treatment, even with 4 guides editing simultaneously, maintained cell viability at close to untreated control samples.

TABLE 42
Relative cell viability 16 hours following treatment with various
editing and delivery conditions

| | | EP | | LNP | |
|---|---|---|---|---|---|
| Editor | Guide(s) | Mean | SD | Mean | SD |
| No Editor | No guide | 100.00 | 2.52 | 100.00 | 5.90 |
| No Editor | G016017 | 13.88 | 1.05 | 89.48 | 1.77 |
| Cas9 | G016017 | 13.25 | 1.18 | 96.43 | 9.82 |
| BC22n | G016017 | 14.78 | 1.37 | 91.20 | 4.67 |
| Cas9 | 4 guides | 13.45 | 0.65 | 98.30 | 4.80 |
| BC22n | 4 guides | 14.38 | 1.49 | 103.40 | 2.75 |

### Example 16.B.5. Staining, Imaging and Quantification of γH2AX Foci

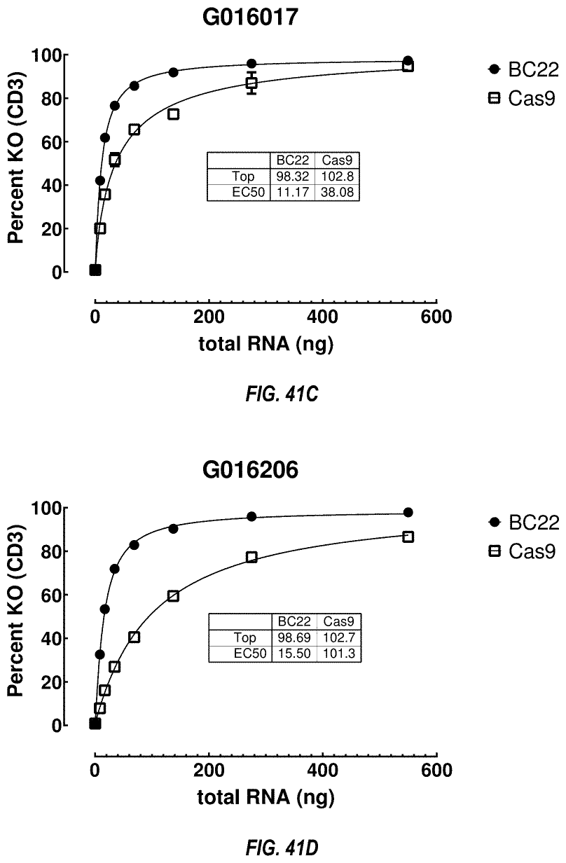

16 hours post electroporation or lipid nanoparticle delivery T cells were cytospun to a slide using Cytospin 4 (Thermo Fisher). After 5 min pre-extraction in PBS/0.5% Trion X-100 on ice, cells were fixed in 4% paraformaldehyde for 10 min. Then, cells were washed in PBS several times and blocked in PBS/0.1% TX-100/1% BSA for 30 min. Primary antibody (Mouse anti-phospho-Histone H2A.X (Ser139) (Millipore Cat. 05-636) was incubated in the blocking buffer at 4° C. overnight. After washed in PBS/0.05% Tween-20 three times, secondary antibody (Goat anti-Mouse IgG Alexa 568 (Thermo Fisher Cat. A31556) was incubated in the blocking buffer at room temperature for 30 min. Cells were washed in PBS/0.05% Tween-20 and nuclei were counter stained with Hoechst 33342. Images were generated by confocal imaging with the Leica SP8. Image analysis was performed via a custom protocol on Thermo Scientific HCS Studio Cell Analysis Software Spot Detector module. Table 43 and FIG. 43 show total γH2AX spot intensity per nuclei following treatment with stated editing and delivery conditions. EP Cas9 with 4 guides samples showed a significant increase in gH2AX foci per nuclei over LNP Cas9-4 guide samples.

TABLE 43
Mean total YH2AX spot intensity per nuclei following treatment
with various editing and delivery conditions

| | No editor | | | Cas9, 4 guides | | | BC22n, 4 guides | | |
|---|---|---|---|---|---|---|---|---|---|
| Delivery | Mean | SD | N | Mean | SD | N | Mean | SD | N |
| Untreated | 134.33 | 95.78 | 5 | | | | | | |
| EP | | | | 21386.62 | 4336.69 | 3 | Not done | | |
| LNP | | | | 2550.88 | 562.77 | 3 | 1770.11 | 291.97 | 5 |

### Example 16.B.6. Flow Cytometry and NGS Sequencing

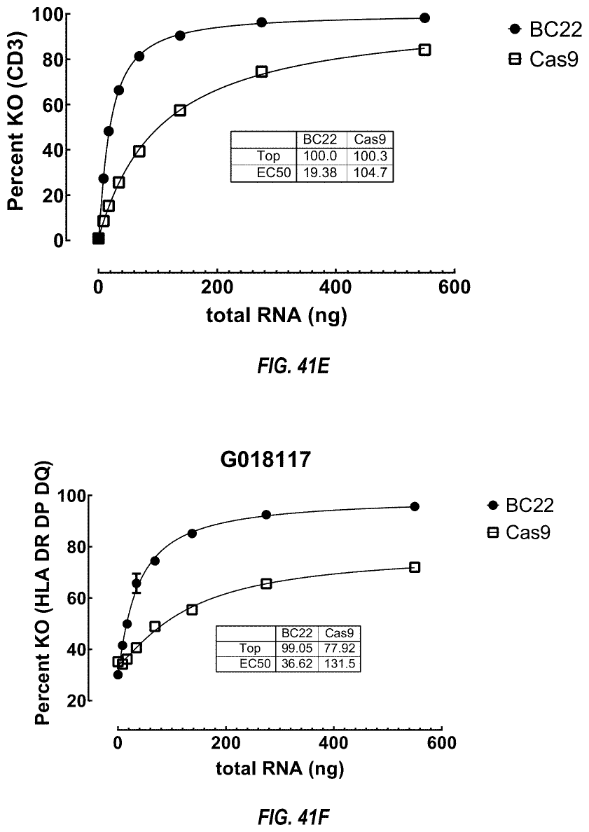

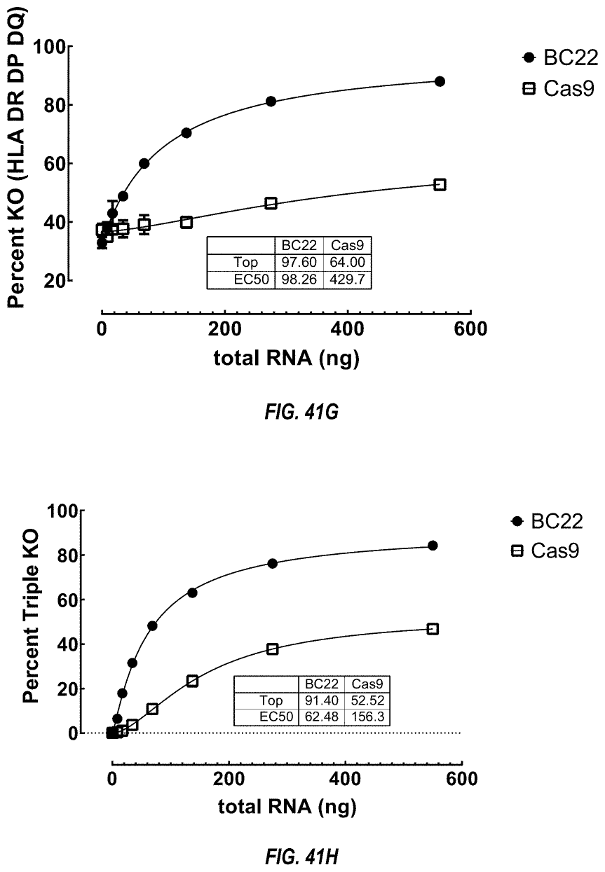

On day 8 post-editing, T cells were phenotyped by flow cytometry to determine B2M, CD3 and HLA II− DR, DP, DQ protein expression. Briefly, T cells were incubated in a cocktail of antibodies targeting B2M-APC/Fire™ 750 (BioLegend® Cat. No. 316314), CD3-BV605 (BioLegend® Cat. No. 316314) and HLA II− DR, DP, DQ-PE (BioLegend® Cat. No. 361716). Cells were subsequently washed, processed on a Cytoflex flow cytometer (Beckman Coulter) and analyzed using the FlowJo software package. T cells were gated based on size, shape, viability, and MHC II expression. DNA samples were subjected to PCR and subsequent NGS analysis, as described in Example 1. Table 44 and FIG. 44 show percent editing at loci of interest following treatment with LNPs. In the condition where 4 guides were delivered by LNP, percent editing is higher at each locus with BC22n than with Cas9. Table 45 and FIG. 45 show surface protein expression of interest following LNP treatment. Editing with BC22n resulted in a greater percentage of triple knockout cells than editing with Cas9.

TABLE 44
Mean percent editing following treatment with stated
editing schemes by LNP delivery.

| | | Readout | C-to-T % | | | C-to-A/G % | | | Indel % | | |
|---|---|---|---|---|---|---|---|---|---|---|---|
| Editor | Guide(s) | Locus | Mean | SD | N | Mean | SD | N | Mean | SD | N |
| No | No | B2M | 0.20 | 0.00 | 2 | 0.41 | 0.01 | 2 | 0.13 | 0.01 | 2 |
| Editor | guide | TRAC | 0.05 | 0.07 | 2 | 0.53 | 0.01 | 2 | 0.09 | 0.01 | 2 |
| | | TRBC1 | 0.25 | 0.07 | 2 | 0.86 | 0.09 | 2 | 0.24 | 0.02 | 2 |
| | | TRBC2 | 0.30 | 0.14 | 2 | 0.85 | 0.04 | 2 | 0.27 | 0.01 | 2 |
| | | CIITA | 0.15 | 0.07 | 2 | 0.26 | 0.03 | 2 | 0.06 | 0.00 | 2 |
| Cas9 | G016017 | TRAC | 0.00 | 0.00 | 2 | 0.05 | 0.00 | 2 | 96.59 | 0.52 | 2 |
| | 4 guides | B2M | 0.30 | 0.14 | 2 | 0.37 | 0.21 | 2 | 83.61 | 2.72 | 2 |
| | | TRAC | 0.00 | 0.00 | 2 | 0.06 | 0.04 | 2 | 89.45 | 0.25 | 2 |
| | | TRBC1 | 0.00 | 0.00 | 2 | 0.35 | 0.01 | 2 | 75.98 | 2.09 | 2 |
| | | TRBC2 | 0.00 | 0.00 | 2 | 0.38 | 0.03 | 2 | 78.07 | 1.51 | 2 |
| | | CIITA | 0.20 | 0.14 | 2 | 0.38 | 0.04 | 2 | 55.23 | 0.92 | 2 |
| BC22n | G016017 | TRAC | 93.90 | 0.57 | 2 | 2.29 | 0.29 | 2 | 2.34 | 0.08 | 2 |
| | 4 guides | B2M | 95.65 | 0.07 | 2 | 1.42 | 0.23 | 2 | 1.07 | 0.16 | 2 |
| | | TRAC | 95.00 | 0.57 | 2 | 1.65 | 0.07 | 2 | 1.44 | 0.43 | 2 |
| | | TRBC1 | 92.40 | 0.00 | 2 | 1.55 | 0.18 | 2 | 1.91 | 0.10 | 2 |
| | | TRBC2 | 92.50 | 0.00 | 2 | 1.62 | 0.02 | 2 | 1.87 | 0.33 | 2 |
| | | CIITA | 91.35 | 0.35 | 2 | 0.93 | 0.04 | 2 | 0.64 | 0.04 | 2 |

TABLE 45
Mean percentage of cells expressing surface following treatment with
stated editing schemes by LNP delivery.

| Editor | Guide | Surface protein | Mean | SD | N |
|---|---|---|---|---|---|
| No | No guide | CD3- | 2.30 | 2.12 | 2 |
| Editor | | B2M- | 4.00 | 2.26 | 2 |
| | | HLA DP DQ DR- | 60.40 | 1.70 | 2 |
| Cas9 | G016017 | CD3- | 97.05 | 0.16 | 2 |
| | 4 guides | B2M- | 77.90 | 4.67 | 2 |
| | | CD3- | 94.54 | 1.34 | 2 |
| | | HLA DP DQ DR- | 70.10 | 4.81 | 2 |
| | | CD3- B2M- HLA DP DQ DR- | 61.58 | 5.42 | 2 |
| BC22n | G016017 | CD3- | 96.20 | 0.13 | 2 |
| | 4 guides | B2M- | 97.35 | 0.37 | 2 |
| | | CD3- | 97.34 | 0.08 | 2 |
| | | HLA DP DQ DR- | 93.46 | 0.89 | 2 |
| | | CD3- B2M- HLA DP DQ DR- | 90.63 | 0.89 | 2 |

### Example 16.B.7. Measuring Structural Variation and Translocations by UnIT

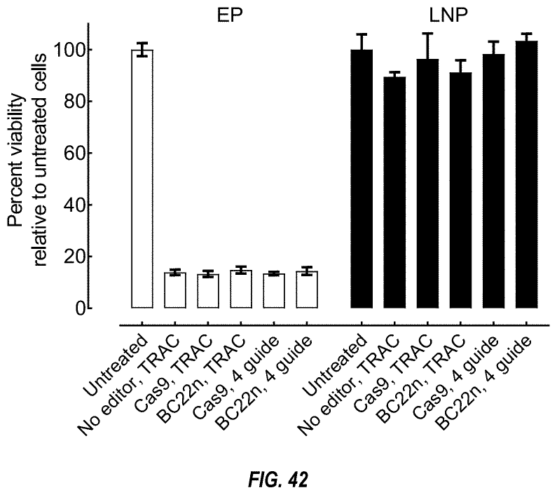

On day 8 post-editing a subset of T cells from the untreated, LNP-Cas9-4 guides and LNP-BC22n-4 guides samples were collected, spun down and resuspended in 100 μL of PBS. gDNA was isolated from the cells using DNeasy Blood & Tissue Kit (Qiagen Cat. 69504). The UnIT structural variant characterization assay was applied to these gDNA samples. High molecular weight genomic DNA is simultaneously fragmented and sequence-tagged (‘tagmented’) with the Tn5 transposase and an adapter with a partial Illumina P5 sequence and a 12 bp unique molecular identifier (UMI). Two sequential PCRs using a primer to P5 and hemi-nested gene specific primers (GSP) imparting the Illumina the P7 sequence to create two Illumina compatible NGS libraries per sample. Sequencing across both directions of the CRISPR/Cas9 targeted cut site with the two libraries allows the inference and quantification of structural variants in DNA repair outcomes after genome editing. If the two fragments were aligned to different chromosomes, the SV was classified as an “inter-chromosomal translocation.” Structural variation results show that interchromosomal translocations are reduced to background levels when multiplex editing is being conducted by BC22n whereas Cas9 multiplex editing leads to significant increases in structural variation, as shown in Table 46 and FIG. 46.

TABLE 46
Mean percent interchromosomal translocations among total unique
molecule identifiers following treatment with stated editing
schemes by LNP delivery.

| Edit | Locus | Mean | SD | N |
|---|---|---|---|---|
| Untreated | B2M | 0.16 | 0.09 | 2 |
| | TRAC | 0.21 | 0.13 | 2 |
| | CIITA | 0.10 | 0.04 | 2 |
| Cas9, 4 guides | B2M | 2.71 | 0.52 | 2 |
| | TRAC | 1.55 | 0.24 | 2 |
| | CIITA | 0.92 | 0.37 | 2 |
| BC22n, 4 guides | B2M | 0.14 | 0.06 | 2 |
| | TRAC | 0.21 | 0.08 | 2 |
| | CIITA | 0.14 | 0.08 | 2 |

### Example 17. Multi-Editing WT1 T Cells with Sequential LNP Delivery

T cells were engineered with a series of gene disruptions and insertions. Healthy donor cells were treated sequentially with four LNPs, each LNP co-formulated with mRNA encoding Cas9 (SEQ ID NO: 6) and a sgRNA targeting either TRAC (G013006) (SEQ ID NO: 708), TRBC (G016239) (SEQ ID NO: 707), CIITA (G013676) (SEQ ID NO: 715), or HLA-A (G018995) (SEQ ID NO: 716). A transgenic T cell receptor targeting Wilms' tumor antigen (WT1 TCR) (SEQ ID NO: 717) was integrated into the TRAC cut site by delivering a homology directed repair template using AAV.

### Example 17.1. T Cell Preparation

T cells were isolated from the leukapheresis products of three healthy HLA-A2+ donors (STEMCELL Technologies). T cells were isolated using EasySep Human T cell Isolation kit (STEMCELL Technologies, Cat. 17951) following manufacturers protocol and cryopreserved using Cryostor CS10 (STEMCELL Technologies, Cat. 07930). The day before initiating T cell editing, cells were thawed and rested overnight in T cell activation media (TCAM): CTS OpTmizer (Thermofisher, Cat. A3705001) supplemented with 2.5% human AB serum (Gemini, Cat. 100-512), 1× GlutaMAX (Thermofisher, Cat. 35050061), 10 mM HEPES (Thermofisher, Cat. 15630080), 200 U/mL IL-2 (Peprotech, Cat. 200-02), IL-7 (Peprotech, Cat. 200-07), IL-15 (Peprotech, Cat. 200-15).

### Example 17.2. LNP Treatment and Expansion of T Cells

LNPs were generally prepared as described in Example 1 at a ratio of 50/10/38.5/1.5 Lipid A, cholesterol, DSPC, and PEG2k-DMG. LNPs were prepared with a ratio of gRNA to mRNA of 1:2 by weight. LNPs were prepared each day in ApoE containing media and delivered to T cells as described in Table 47 and below.

TABLE 47
Order of editing for T cell engineering

| Group | Day 1 | Day 2 | Day 3 | Day 4 |
|---|---|---|---|---|
| 1 | Unedited | Unedited | Unedited | Unedited |
| 2 | TRBC | CIITA | TRAC | HLA-A |
| 3 | TRBC | HLA-A | TRAC | CIITA |
| 4 | TRBC | | TRAC | |

On day 1, LNPs as indicated in Table 47 were incubated at a concentration of 5 ug/mL in TCAM containing 5 ug/mL rhApoE3 (Peprotech, Cat. 350-02). Meanwhile, T cells were harvested, washed, and resuspended at a density of 2×10⁶ cells/mL in TCAM with a 1:50 dilution of T Cell TransAct, human reagent (Miltenyi, Cat. 130-111-160). T cells and LNP-ApoE media were mixed at a 1:1 ratio and T cells plated in culture flasks overnight.

On day 2, LNPs as indicated in Table 47 were incubated at a concentration of 25 ug/mL in TCAM containing 20 ug/mL rhApoE3 (Peprotech, Cat. 350-02). LNP-ApoE solution was then added to the appropriate culture at a 1:10 ratio.

On day 3, TRAC-LNPs were incubated at a concentration of 5 ug/mL in TCAM containing 10 ug/mL rhApoE3 (Peprotech, Cat. 350-02). T cells were harvested, washed, and resuspended at a density of 1×10⁶ cells/mL in TCAM. T cells and LNP-ApoE media were mixed at a 1:1 ratio and T cells plated in culture flasks. WT1 AAV (SEQ ID NO: 717) was then added to each group at a MOI of 3×10⁵ genome copies/cell.

On day 4, LNPs as indicated in Table 47 were incubated at a concentration of 5 ug/mL in TCAM containing 5 ug/mL rhApoE3 (Peprotech, Cat. 350-02). LNP-ApoE solution was then added to the appropriate culture at a 1:1 ratio.

On days 5-11, T cells were transferred to a 24-well GREX plate (Wilson Wolf, Cat. 80192) in T cell expansion media (TCEM): CTS OpTmizer (Thermofisher, Cat. A3705001) supplemented with 5% CTS Immune Cell Serum Replacement (Thermofisher, Cat.

A2596101), 1× GlutaMAX (Thermofisher, Cat. 35050061), 10 mM HEPES (Thermofisher, Cat. 15630080), 200 U/mL IL-2 (Peprotech, Cat. 200-02), IL-7 (Peprotech, Cat. 200-07), and IL-15 (Peprotech, Cat. 200-15). Cells were expanded per manufacturers protocols. T cells were expanded for 6-days, with media exchanges every other day. Cells were counted using a Vi-CELL cell counter (Beckman Coulter) and fold expansion was calculated by dividing cell yield by the starting material as shown in Table 48.

TABLE 48
Fold expansion following multi-edit T cell engineering

| Group | Donor A | Donor B | Donor C | Mean | SD |
|---|---|---|---|---|---|
| 1 | 331.40 | 362.24 | 533.18 | 408.94 | 108.69 |
| 2 | 61.82 | 72.15 | 116.13 | 83.37 | 28.84 |
| 3 | 64.08 | 76.29 | 157.75 | 99.37 | 50.92 |
| 4 | No data | 146.78 | 331.67 | 239.22 | 130.74 |

### Example 17.3. Quantification of T Cell Editing by Flow Cytometry and NGS

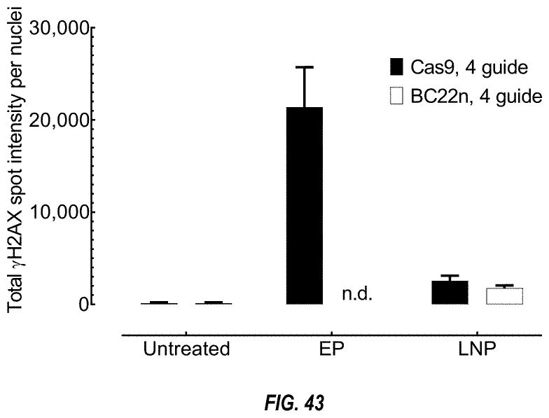

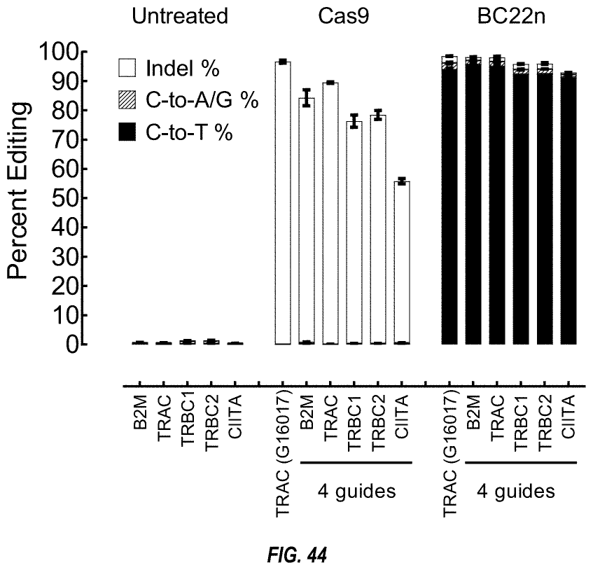

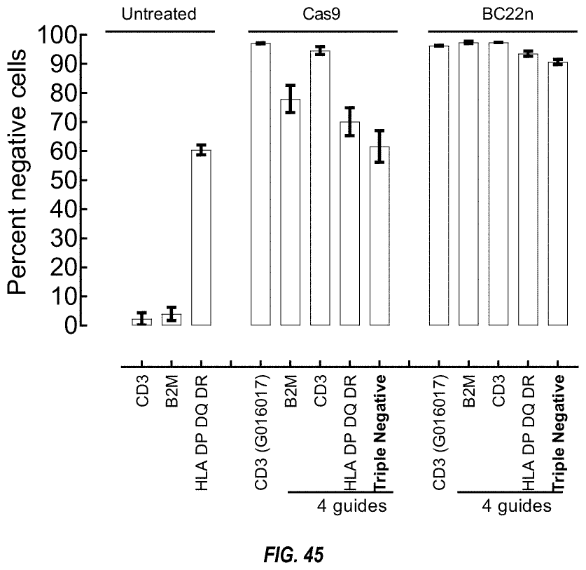

Post expansion, edited T cells were assayed by flow cytometry to determine HLA-A2 expression (HLA-A⁺), HLA-DR-DP-DQ expression (MHC II⁺) following knockdown CIITA, WT1-TCR expression (CD3⁺ Vb8⁺), and the expression of residual endogenous TCRs (CD3⁺ Vb8⁻) or mispaired TCRs (CD3⁺ Vb8^{low}). T cells were incubated with an antibody cocktail targeting the following molecules: CD4 (Biolegend, Cat. 300524), CD8 (Biolegend, Cat. 301045), Vb8 (Biolegend, Cat. 348106), CD3 (Biolegend, Cat. 300327), HLA-A2 (Biolegend, Cat. 343306), HLA-DRDPDQ (Biolegend, Cat 361706), CD62L (Biolegend, Cat. 304844), CD45RO (Biolegend, Cat. 304230). Cells were subsequently washed, analyzed on a Cytoflex LX instrument (Beckman Coulter) using the FlowJo software package. T cells were gated on size and CD4/CD8 status, before expression of editing and insertion markers was determined. The percentage of cells expressing relevant cell surface proteins following sequential T cell engineering are shown in Table 49 and FIGS. 47A-F for CD8⁺ T cells and Table 50 and FIGS. 48A-F for CD4⁺ T cells. The percent of fully edited CD4⁺ or CD8⁺ T cells was gated as % CD3⁺ Vb8⁺ HLA-A⁻ WIC IP. High levels of HLA-A and MHC II knockdown, as well as WT1-TCR insertion and endogenous TCR KO are observed in edited samples. In addition to flow cytometry analysis, genomic DNA was prepared and NGS analysis performed as described in Example 1 to determine editing rates at each target site. Table 51 and FIGS. 49A-D show results for percent editing at the CIITA, HLA-A, and TRBC1/2 loci, with patterns across the groups consistent with what was identified by flow cytometry. TRBC1/2 loci were edited to >90-95% in all groups.

TABLE 49
Percentage of CD8+ cell with cell surface
phenotype following sequential T cell engineering

| | | | | | | % | % Fully |
|---|---|---|---|---|---|---|---|
| | | | | | | Resi- | edited |
| | | | % | | % | dual | CD3⁺ |
| | | % | MHC | % | Mis- | endo- | Vb8⁺ |
| | | HLA- | II* | WT1 | paired | genous | HLA-A2 |
| | | A2⁺ | HLA- | TCR | TCR | TCR | HLA- |
| | | HLA- | DR- | CD3⁺ | CD3⁺ | CD3⁺ | DR- |
| Donor | Group | A2⁺ | DP-DQ⁺ | Vb8⁺ | Vb8^{low} | Vb8⁻ | DP-DQ |
| A | 1 | 100.0 | 60.9 | 6.7 | 0.8 | 93.2 | 0.0 |
| B | Un- | 99.7 | 71.0 | 3.4 | 0.6 | 96.1 | 0.2 |
| C | edited | 99.7 | 52.2 | 5.7 | 0.8 | 94.0 | 0.0 |
| A | 2 | 2.7 | 1.2 | 68.9 | 1.3 | 0.4 | 66.7 |
| B | | 1.3 | 21.0 | 50.4 | 3.1 | 4.5 | 43.3 |
| C | | 1.8 | 2.9 | 62.2 | 2.6 | 2.7 | 60.3 |
| A | 3 | 1.3 | 0.8 | 66.0 | 1.4 | 0.3 | 64.4 |
| B | | 1.4 | 2.2 | 56.8 | 2.2 | 2.0 | 55.1 |
| C | | 1.2 | 5.7 | 63.3 | 1.0 | 0.9 | 60.6 |
| B | 4 | 99.8 | 64.8 | 62.3 | 2.0 | 2.5 | 0.1 |
| C | | 99.0 | 51.5 | 71.0 | 1.0 | 0.5 | 0.4 |

TABLE 50
Percentage of CD4+ cells with cell surface
phenotype following sequential T cell engineering

| | | | | | | % | |
|---|---|---|---|---|---|---|---|
| | | | | | | Resi- | % Fully |
| | | | % | | % | dual | edited |
| | | % | MHC | % | Mis- | endo- | CD3⁺ |
| | | HLA- | II⁺ | WT1 | paired | genous | Vb8⁺ |
| | | A⁺ | HLA- | TCR | TCR | TCR | HLA-A2⁻ |
| | | HLA- | DR- | CD3⁺ | CD3⁺ | CD3⁺ | HLA-DR- |
| Donor | Group | A2⁺ | DP-DQ⁺ | Vb8⁺ | Vb8^{low} | Vb8⁻ | DP-DQ |
| A | 1 | 100.0 | 36.3 | 5.4 | 0.4 | 94.5 | 0.0 |
| B | Un- | 98.7 | 27.6 | 5.6 | 0.4 | 94.3 | 0.0 |
| C | edited | 99.3 | 32.3 | 6.2 | 0.3 | 93.6 | 0.1 |
| A | 2 | 2.6 | 0.7 | 62.4 | 2.4 | 1.1 | 60.9 |
| B | | 1.8 | 0.5 | 59.7 | 2.2 | 1.0 | 58.5 |
| C | | 1.7 | 3.2 | 58.6 | 1.6 | 1.8 | 55.8 |
| A | 3 | 1.3 | 0.8 | 63.0 | 3.4 | 0.8 | 61.7 |
| B | | 1.1 | 1.1 | 61.8 | 2.6 | 0.9 | 60.6 |
| C | | 1.1 | 0.4 | 60.9 | 1.7 | 1.0 | 59.9 |
| B | 8 | 99.5 | 25.1 | 61.9 | 1.9 | 5.2 | 0.1 |
| C | | 97.9 | 40.1 | 69.5 | 4.7 | 1.9 | 0.8 |

TABLE 51
Percent indels at CIITA, HLA-A, TRBC1 and TRBC2 following
sequential T cell editing

| | CIITA | | | HLA-A | | | TRBC1 | | | TRBC2 | | |
|---|---|---|---|---|---|---|---|---|---|---|---|---|
| | (G013676) | | | (G018995) | | | (G016239) | | | (G016239) | | |
| | Don | Don | Don | Don | Don | Don | Don | Don | Don | Don | Don | Don |
| Group | or A | or B | or C | or A | or B | or C | or A | or B | or C | or A | or B | or C |
| 1 | 0.2 | 0.2 | 0.2 | 6.9 | 3.3 | 2.3 | 0.1 | 0.3 | 0.2 | 0.3 | 0.3 | 0.3 |
| 2 | 98.2 | 81.8 | 93.8 | 94.1 | 90.2 | 90.6 | 97.6 | 89.9 | 91.4 | 98.7 | 86.8 | 94.9 |
| 3 | 98.9 | 98.1 | 98.9 | 97.2 | 86.4 | 93.1 | 98.6 | 94.4 | 94.7 | 98.6 | 94.2 | 96.6 |
| 4 | 0.1 | 0.2 | 0.6 | 7.6 | 2.7 | 3.2 | 98.9 | 94 | 95 | 98.6 | 93.2 | 97.4 |

### Example 18. Multi-Editing with Two Insertions in T Cells

To demonstrate engineering of T cells with five distinct Cas9 edits, healthy donor cells were treated sequentially with five LNPs co-formulated with an mRNA encoding Cas9 (SEQ ID NO. 6) and a sgRNA targeting either TRAC (G013006) (SEQ ID NO: 708), TRBC (G016239) (SEQ ID NO: 707), CIITA (G013676) (SEQ ID NO: 715), HLA-A (G018995) (SEQ ID NO: 716), or AAVS1(G000562) (SEQ ID NO: 710). A transgenic WT1 targeting TCR was site-specifically integrated into the TRAC cut site by delivering a homology directed repair template (SEQ ID NO. 717) using AAV. As a proof-of-concept GFP was site-specifically integrated into the AAVS1 target site using a second homology repair template (SEQ ID NO. 720).

T cells were isolated from the leukapheresis products of two healthy HLA-A*02:01+ donors (STEMCELL Technologies). T cells were isolated using EasySep Human T cell Isolation kit (STEMCELL Technologies, 17951) following manufacturer's protocol and cryopreserved using Cryostor CS10 (STEMCELL Technologies, 07930). The day before initiating T cell editing, cells were thawed and rested overnight in T cell activation media (TCAM: CTS OpTmizer (Thermofisher, A3705001) supplemented with 2.5% human AB serum (Gemini, 100-512), 1× GlutaMAX (Thermofisher, 35050061), 10 mM HEPES (Thermofisher, 15630080), 200 U/mL IL-2 (Peprotech, 200-02), 5 ng/mL IL7 (Peprotech, 200-07), and 5 ng/mL IL-15 (Peprotech, 200-15).

### Example 18.1. LNP Treatment and Expansion of T Cells

LNPs were generally prepared as described in Example 1 with the lipid composition of 50/10/38.5/1.5, expressed as the molar ratio of ionizable lipid/cholesterol/DSPC/PEG, respectively. Immediately prior to exposure to T cells, LNPs were preincubated in ApoE containing media. Experimental design of the sequential editing steps and control groups is found in Table 52.

TABLE 52
Experimental Design

| Group | Day 1 | Day 2 | Day 3 | Day 4 | Day 5 | Purpose |
|---|---|---|---|---|---|---|
| Unedited | None | None | None | None | None | Negative |
| | | | | | | control |
| GFP- | None | None | None | AAV- | None | Control for |
| AAV | | | | GFP | | GFP |
| only | | | | | | episomal |
| | | | | | | expression |
| TCR | None | None | TRAC | None | TRBC | Control |
| edits | | | LNP + | | | for TCR |
| only | | | WT1 | | | replacement |
| | | | AAV | | | |
| Quintuple | CIITA | HLA- | TRAC | AAVS1 + | TRBC | Ex- |
| Edit | | A | LNP + | AAV- | | perimental |
| | | | WT1 | GFP | | |
| | | | AAV | | | |

Day 1: LNPs targeting CIITA as indicated in Table 52 were incubated at a concentration of 5 ug/mL in TCAM containing 5 ug/mL rhApoE3 (Peprotech 350-02). T cells were harvested, washed, and resuspended at a density of 2×10\hat{ }6 cells/mL in TCAM with a 1:50 dilution of T Cell TransAct, human reagent (Miltenyi, 130-111-160). T cells and LNP-ApoE solutions were then mixed at a 1:1 ratio and T cells plated in culture flasks overnight.

Day 2: LNPs targeting HLA-A as indicated in Table 52 were incubated at a concentration of 25 ug/mL in TCAM containing 20 ug/mL rhApoE3 (Peprotech 350-02). LNP-ApoE solution was then added to the appropriate culture at a 1:10 ratio by volume.

Day 3: LNPs targeting TRAC were incubated at a concentration of 5 ug/mL in TCAM containing 5 ug/mL rhApoE3 (Peprotech 350-02). T cells were harvested, washed, and resuspended at a density of 1×10\hat{ }6 cells/mL in TCAM. T cells and LNP-ApoE media were mixed at a 1:1 ratio by volume and T cells plated in culture flasks. WT1 AAV was then added to each group at a MOI of 3×10\hat{ }5 GCU/cell. The DNA-PK inhibitor Compound 4 was added to each group at a concentration of 0.25 μM

Day 4: LNPs targeting AAVS1 were incubated at a concentration of 5 ug/mL in TCAM containing 5 ug/mL rhApoE3 (Peprotech 350-02). Meanwhile, T cells were harvested, washed, and resuspended at a density of 1×10\hat{ }6 cells/mL in TCAM. T cells and LNP-ApoE media were mixed at a 1:1 ratio by volume was added to each group at a concentration of 0.25

Day 5: LNPs targeting TRBC as indicated in Table 52 were incubated at a concentration of 5 ug/mL in TCAM containing 5 ug/mL rhApoE3 (Peprotech 350-02). T cells were harvested, washed, and resuspended at a density of 1×10\hat{ }6 cells/mL in TCAM. LNP-ApoE solution was then added to the appropriate culture at a 1:1 ratio by volume.

Day 6-11: T cells were transferred to a 24-well GREX plate (Wilson Wolf, 80192) in T cell expansion media (TCEM: CTS OpTmizer (Thermofisher, A3705001) supplemented with 5% CTS Immune Cell Serum Replacement (Thermofisher, A2596101), 1× GlutaMAX (Thermofisher, 35050061), 10 mM HEPES (Thermofisher, 15630080), 200 U/mL IL-2 (Peprotech, 200-02), 5 ng/ml IL7 (Peprotech, 200-07), 5 ng/ml IL-15 (Peprotech, 200-15)) and expanded per manufacturer's protocols. Briefly, T cells were expanded for 6 days, with media exchanges every other day.

### Example 18.2. Quantification of T Cell Editing by Flow Cytometry and NGS

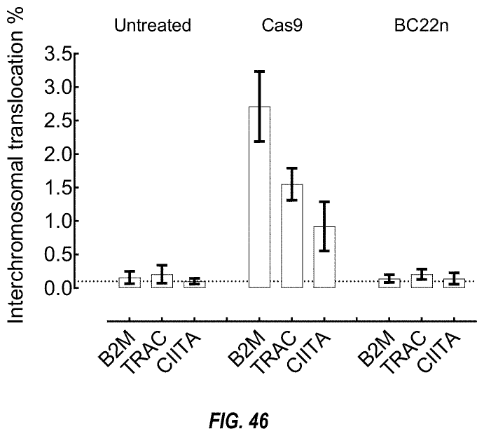

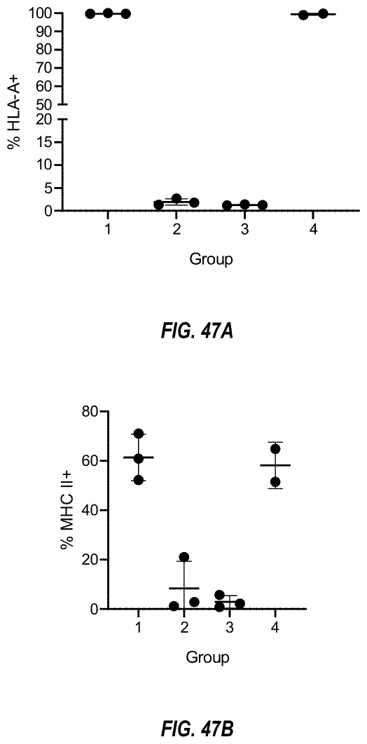

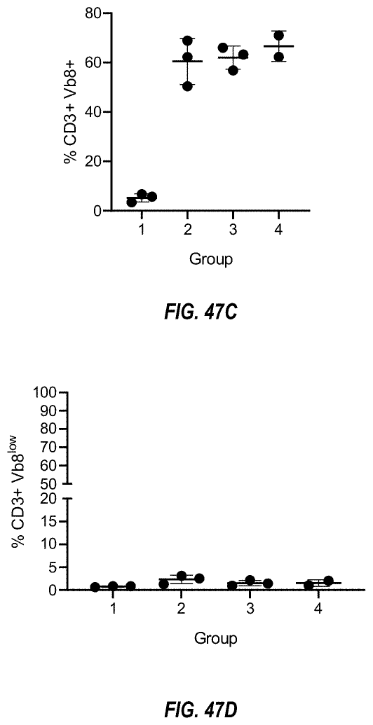

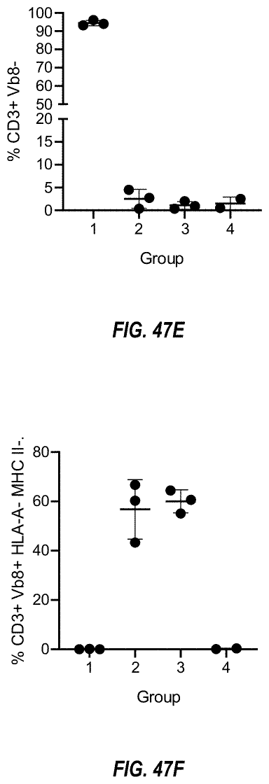

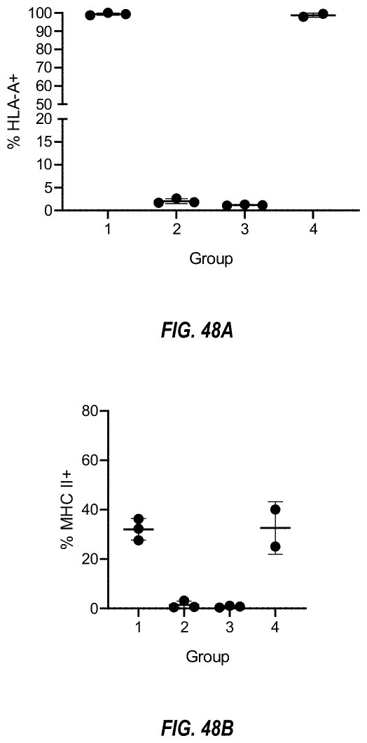

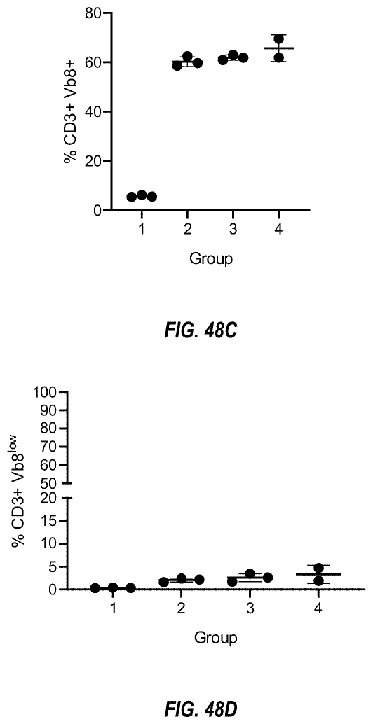

Post expansion, edited T cells were stained with antibodies targeting HLA-A*02:01 (Biolegend 343307), HLA-DR-DP-DQ (Biolegend 361712), WT1-TCR (Vb8+, Biolegend 348104), CD3e (Biolegend 300328), CD4 (Biolegend 317434), CD8 (Biolegend 301046), and Viakrome 808 Live/Dead (Cat. C36628). This cocktail was used to determine HLA-A*02:01 knockout (HLA-A2−), HLA-DR-DP-DQ knockdown via CIITA knockout (HLA-DRDPDQ-), WT1-TCR insertion (CD3+Vb8+), and the percentage of cells expressing residual endogenous TCR (CD3+Vb8−),Insertion into the AAVS1 site was tracked by monitoring GFP expression. Following antibody incubation, cells were washed, processed on a Cytoflex LX instrument (Beckman Coulter) and analyzed using the FlowJo software package. T cells were gated on size and CD4/CD8 status prior to examining editing and insertion markers. Editing and insertion rates can be found in Tables 53 & 54 for CD8+ and CD4+ T cells, respectively. FIGS. 50A-F show graphs of the editing rates of all targets in CD8+ T cells. The percent of T cells with all intended edits (i.e., insertion of the WT1-TCR and GFP, combined with knockout of HLA-A and CIITA) was gated as % CD3+Vb8+ GFP+ HLA-A− HLA-DRDPDQ-. High levels of HLA-A and CIITA knockout, as well as GFP and WT1-TCR insertion were observed in quintuple edited samples from both donors, yielding >75% of fully edited CD8+ T cells and >85% of fully edited CD4+ T cells.

TABLE 53
Editing rates in CD8+ T cells in Donors A and B

| | | | GFP- | | TCR | | | |
|---|---|---|---|---|---|---|---|---|
| | | | AAV | | edits | | Quintuple | |
| | Unedited | | only | | only | | Edit | |
| Marker | A | B | A | B | A | B | A | B |
| GFP+ | 0.0 | 0.0 | 0.3 | 0.7 | 0.0 | 0.0 | 88.4 | 93.1 |
| HLA-A2− | 0.1 | 0.0 | 0.0 | 0.0 | 0.3 | 0.2 | 99.7 | 99.4 |
| HLA-DRDPDQ− | 31.9 | 29.6 | 30.0 | 32.0 | 40.5 | 56.7 | 99.0 | 98.7 |
| CD3+Vb8− | 93.3 | 96.4 | 94.1 | 95.8 | 0.2 | 0.3 | 1.0 | 0.8 |
| CD3+Vb8+ | 6.0 | 3.5 | 5.5 | 4.0 | 92.3 | 94.3 | 86.7 | 92.1 |
| CD3+Vb8+ | 0.0 | 0.0 | 0.0 | 0.0 | 0.0 | 0.0 | 76.7 | 84.8 |
| GFP+ HLA-A−, | | | | | | | | |
| HLA-DRDPDQ− | | | | | | | | |

TABLE 54
Editing rates in CD4+ T cells in Donors A and B

| | | | GFP- | | TCR | | | |
|---|---|---|---|---|---|---|---|---|
| | | | AAV | | edits | | Quintuple | |
| | Unedited | | only | | only | | Edit | |
| Marker | A | B | A | B | A | B | A | B |
| GFP+ | 0.0 | 0.0 | 0.9 | 1.3 | 0.0 | 0.0 | 86.4 | 91.3 |
| HLA-A2− | 0.1 | 0.0 | 0.0 | 0.1 | 0.2 | 0.2 | 99.5 | 99.2 |
| HLA-DRDPDQ− | 77.0 | 73.6 | 71.3 | 75.0 | 93.1 | 96.1 | 99.2 | 99.1 |
| CD3+Vb8− | 94.6 | 94.6 | 94.8 | 94.2 | 0.3 | 0.4 | 1.3 | 1.3 |
| CD3+Vb8+ | 5.1 | 5.1 | 4.9 | 5.4 | 86.3 | 90.9 | 80.7 | 91.0 |
| Vb8+ GFP+ | 0.6 | 0.0 | 0.1 | 0.0 | 0.0 | 0.0 | 86.1 | 90.6 |
| HLA-A− | | | | | | | | |
| HLA-DRDPDQ− | | | | | | | | |

### Example 19. Editing Efficiency with Various APO Proteins in Activated and Non-Activated T Cells

To evaluate editing efficacy, LNPs targeting TRBC were pre-incubated with ApoE3, ApoE4 or ApoA1 in varying concentrations prior to exposure to activated or non-activated T cells. Editing was assayed by an increase in the percentage of CD3 negative cells following editing. The T cell receptor beta chain encoded by TRBC and CD3 are both required parts of the T cell receptor complex at the cell surface. Accordingly, disruption of the TRBC gene by genome editing leads to a loss of CD3 protein on the cell surface of T cells.

Healthy human donor leukopak was obtained commercially (Hemacare) and T cells were isolated by CD4/CD8 positive selection using the StraightFrom® Leukopak® CD4/CD8 MicroBeads (Miltenyi, Catalog, 130-122-352) following the manufacturer's protocol on MultiMACS Cell24 Separator Plus instrument. T cells were aliquoted into vials and cryopreserved in Cryostor CS10 freezing media (Catalog, 07930) for future use.

Upon thaw, T cells were cultured in complete T cell media: T cell base media composed of XVIVO-15 media (Fisher, BE02-060Q), 1% Pen-Strep (Corning, 30-002-CI), 50 uM beta-mercaptoethanol, and N-Acetyl L-Cysteine (Fisher, ICN19460325), which was further supplemented with 5% human AB serum (Gemini Bio Products, 100-512), 200 U/mL IL-2 (Peprotech, 200-02), 5 ng/mL IL7 (Peprotech, 200-07), 5 ng/mL IL-15 (Peprotech, 200-15)). At this stage, a portion of cells were activated by addition of 1:100 dilution of TransAct (Miltenyi Biotech, Catalog #130-111-160). All cells were cultured at 37 C for 48 hours. 100,000 T cells were resuspended in complete T cell media without human serum for 15-30 min prior to LNP transfection.

After 48 hours in culture, activated and non-activated T cells were treated with LNPs delivering mRNA encoding Cas9 (SEQ ID NO. 6) and sgRNA targeting TRBC (G016239) (SEQ ID NO: 707). LNPs were generally prepared as Example 1 with the lipid composition of 50/9/39.5/1.5, expressed as the molar ratio of ionizable lipid A/cholesterol/DSPC/PEG, respectively. Immediately prior to exposure to T cells, LNPs were preincubated at 37 C for about 5 to 15 minutes with Recombinant Human ApoE3 (Peprotech, Cat #350-02), Recombinant Human ApoE4 (Novus Biologicals, Cat #NBP1-99634-1000 ug), or Recombinant Human ApoA1 (Novus Biologicals, Cat #NBP2-34869-500 ug) in concentrations of 10, 5, 2.5, 1.25, 0.63, 0.31, 0.16, and 0.08 ug/mL in T cell media without serum After 5-15 minutes of incubation with recombinant Apo protein, LNPs were added to 100,000 T cells at a dose of 4 ug/mL of total RNA cargo (1:2 w/w ratio of Cas9 mRNA and single guide). Cells were washed 48 hr post LNP treatment with T cell media to wash and replaced with fresh complete T cell media.

Five days post LNP treatment, T cells were phenotyped by flow cytometry to determine CD3 protein surface expression. Briefly, T cells were incubated in antibody targeting CD3 (Biolegend, 300441). Cells were subsequently washed, analyzed on a CytoFLEX S instrument (Beckman Coulter) using the FlowJo software package. T cells were gated on size and CD3 expression. Table 55 shows the percent of CD3 negative cells following LNP treatment of activated T cells. Table 56 shows percent CD3 negative cells following LNP treatment of non-activated T cells. In both activated and non-activated T cells, ApoE3 and ApoE4 exposure led to efficient editing in a dose-dependent manner. Conversely, none of the tested concentrations of ApoA1 protein led to efficient editing and subsequent decrease in CD3 surface expression.

TABLE 55
Percent CD3 negative cells after activated T
cells were treated with LNPs preincubated
with stated levels of Apo protein.

| | ApoE3 | | ApoE4 | | ApoA1 | |
|---|---|---|---|---|---|---|
| Apo | Bio- | Bio- | Bio- | Bio- | Bio- | Bio- |
| proteins | logical | logical | logical | logical | logical | logical |
| (ug/ | Repli- | Repli- | Repli- | Repli- | Repli- | Repli- |
| mL) | cate1 | cate2 | cate1 | cate2 | cate1 | cate2 |
| 10.00 | 90.2 | 90.8 | 89.8 | 91.7 | 3.6 | 3.6 |
| 5.00 | 90.2 | 90.7 | 91.0 | 91.2 | 2.5 | 3.2 |
| 2.50 | 90.2 | 91 | 91.4 | 91.0 | 4.0 | 2.9 |
| 1.25 | 89.5 | 89.7 | 90.4 | 89.4 | 2.8 | 3.0 |
| 0.63 | 86.4 | 88.0 | 85.9 | 87.5 | 3.1 | 2.6 |
| 0.31 | 82.6 | 82.2 | 72.3 | 75.0 | 2.9 | 2.1 |
| 0.16 | 70.9 | 69.4 | 37.2 | 39.2 | 2.5 | 2.7 |
| 0.08 | 51.1 | 49.0 | 17.6 | 18.8 | 2.9 | 2.5 |

TABLE 56
Percent CD3 negative cells after non-activated T
cells were treated with LNPs preincubated with
stated levels of Apo protein.

| Apo | ApoE3 | | ApoE4 | | ApoA1 | |
|---|---|---|---|---|---|---|
| pro- | Bio- | Bio- | Bio- | Bio- | Bio- | Bio- |
| teins | logical | logical | logical | logical | logical | logical |
| (ug/ | Repli- | Repli- | Repli- | Repli- | Repli- | Repli- |
| mL) | cate1 | cate2 | cate1 | cate2 | cate1 | cate2 |
| 10.00 | 78.7 | 76.8 | 82.8 | 79.9 | 5.4 | 3.8 |
| 5.00 | 69.0 | 66.5 | 76.3 | 78.9 | 6.1 | 4.8 |
| 2.50 | 60.3 | 61.6 | 73.6 | 77.4 | 6.8 | 3.9 |
| 1.25 | 58.4 | 63.5 | 74.3 | 78.5 | 4.7 | 4.2 |
| 0.63 | 58.3 | 58.4 | 72.2 | 72.5 | 6.5 | 6.0 |
| 0.31 | 60.6 | 61.8 | 63.5 | 67.0 | 4.4 | 3.8 |
| 0.16 | 58.5 | 56.6 | 52.7 | 54.5 | 5.2 | 3.8 |
| 0.08 | 54.7 | 56.3 | 49.9 | 43.4 | 4.2 | 6.2 |

### Example 20. Editing Efficiency with Different Ionizable Lipids in Activated and Non-Activated T Cells

To assess efficient nucleic acid delivery, activated and non-activated T cells were treated with LNPs formulated with different ionizable lipids and Cas9 protein expression or the percent of CD3 negative cells was measured.

T cells were isolated as in Example 19. Upon thaw, T cells were cultured in T cell base media composed media composed of CTS OpTmizer (Thermofisher, A10485-01), 1% pen-strep (Corning, 30-002-CI) 1× GlutaMAX (Thermofisher, 35050061), 1% pen-strep (Corning, 30-002-CI) 1× GlutaMAX (Thermofisher, 35050061), 10 mM HEPES (Thermofisher, 15630080)) which was further supplemented with 5% human AB serum (Gemini, 100-512), 200 U/mL IL-2 (Peprotech, 200-02), 5 ng/ml IL7 (Peprotech, 200-07), 5 ng/ml IL-15 (Peprotech, 200-15). At this stage, a portion of cells were activated by addition of 1:100 dilution of TransAct (Miltenyi Biotech, Catalog #130-111-160) All cells were cultured at 37 C for 24 hours. One hundred thousand T cells were resuspended in T cell base media composed of CTS OpTmizer (Thermofisher, A10485-01), 1% pen-strep (Corning, 30-002-CI) 1× GlutaMAX (Thermofisher, 35050061), 10 mM HEPES (Thermofisher, 15630080)) which was further supplemented 200 U/mL IL-2 (Peprotech, 200-02), 5 ng/mL IL7 (Peprotech, 200-07), 5 ng/mL IL-15 (Peprotech, 200-15) without Human Serum for 15-30 min prior to LNP transfection.

### Example 20.1. Cas9 Expression in Activated and Non-Activated T Cells

After 24 hours in culture, activated and non-activated T cells were treated with LNPs delivering mRNA encoding Hibit-Cas9 (SEQ ID NO. 7) and no sgRNA. LNPs were generally prepared as in Example 1 with the ionizable lipids indicated in Table 57 in a lipid composition of 50/10/38.5/1.5, expressed as the molar ratio of ionizable lipid/cholesterol/DSPC/PEG, respectively. Immediately prior to exposure to T cells, LNPs were preincubated at 37 C for about 5-15 minutes at a LNP concentration of 20 ug/ml total RNA cargo with 20 ug/mL ApoE3 (Peprotech, Cat #350-02) in T cell base media composed of CTS OpTmizer (Thermofisher, A10485-01), 1% pen-strep (Corning, 30-002-CI) 1× GlutaMAX (Thermofisher, 35050061), 10 mM HEPES (Thermofisher, 15630080)) which was further supplemented with 5% human AB serum (Gemini, 100-512), 200 U/mL IL-2 (Peprotech, 200-02), 5 ng/mL IL7 (Peprotech, 200-07), 5 ng/mL IL-15 (Peprotech, 200-15). After preincubation, LNPs were added to 100,000 T cells. Forty-eight hours post LNP treatment, T cells were harvested for protein expression.

Harvested T cells were lysed by Nano-Glo® HiBiT Lytic Assay (Promega). Cas9 protein levels were determined by using Nano-Glo® HiBiT Extracellular Detection System (Promega, Cat. N2420) following the manufacturer's protocol. Luminescence was measured using the Biotek Neo2 plate reader. Linear regression was plotted on GraphPad using the protein number and luminescence readouts from the standard controls, forcing the line to go through X=0, Y=0. Used the Y=ax+0 equation to calculate number of proteins per lysate. Samples were normalized to the mean of activated cell, 1.25 ug/ml LIPID A formulation samples. Table 57 shows the relative Cas9 protein expression in activated and non-activated cells when mRNA is delivered with LNPs composed with different ionizable lipids. Cas9 was expressed in a dose dependent manner under both formulation conditions and in activated and non-activated cells. Protein expression was higher in activated cells for both formulations tested.

TABLE 57
Relative Cas9 protein expression

| Cell | LNP | Lipid A | | Lipid D | |
|---|---|---|---|---|---|
| Condition | (ug/ml) | Mean | SD | Mean | SD |
| Activated | 20.00 | 28.62 | 6.98 | 12.00 | 2.90 |
| | 10.00 | 20.73 | 0.29 | 5.92 | 0.32 |
| | 5.00 | 14.91 | 0.17 | 2.79 | 0.21 |
| | 2.50 | 6.33 | 1.05 | 0.93 | 0.08 |
| | 1.25 | 1.00 | 0.05 | 0.34 | 0.04 |
| | 0.63 | 0.19 | 0.04 | 0.15 | 0.01 |
| | 0.31 | 0.05 | 0.01 | 0.08 | 0.01 |
| | 0.00 | 0.00 | 0.00 | 0.00 | 0.00 |
| Non- | 20.00 | 0.81 | 0.12 | 0.23 | 0.00 |
| activated | 10.00 | 0.69 | 0.02 | 0.11 | 0.02 |
| | 5.00 | 0.68 | 0.07 | 0.09 | 0.01 |
| | 2.50 | 0.35 | 0.00 | 0.03 | 0.00 |
| | 1.25 | 0.08 | 0.01 | 0.01 | 0.00 |
| | 0.63 | 0.01 | 0.00 | 0.01 | 0.00 |
| | 0.31 | 0.00 | 0.00 | 0.01 | 0.00 |
| | 0.00 | 0.01 | 0.01 | 0.00 | 0.00 |

### Example 20.2. Evaluating Editing with mRNA and Guide RNA Delivery Separated in Time in Non-Activated T Cells

To evaluate editing efficacy when Cas9 mRNA and sgRNA are delivered at separate times, T cells were treated with LNPs in which Cas9 mRNA and a sgRNA targeting TRAC were formulated separately. Editing was assayed by an increase in the percentage of CD3 negative cells following editing. The T cell receptor alpha chain encoded by TRAC is required for T cell receptor/CD3 complex assembly and translocation to the cell surface. Accordingly, disruption of the TRAC gene by genome editing leads to a loss of CD3 protein on the cell surface of T cells.

T cells were isolated and prepared as in Example 19. After 24 hours in culture, non-activated T cells were treated with LNPs delivering either mRNA encoding Cas9 (SEQ ID NO. 7) only or LNP co-formulated to deliver both mRNA encoding Cas9 and sgRNA G013006 (SEQ ID NO: 708) targeting TRAC. Subsequently, engineered T cells were treated at 0 hours or 72 hours after the initial LNP treatment with a second LNP formulated with Lipid A and PEG-DMG delivering only sgRNA G013006 (SEQ ID NO: 708). LNPs were generally prepared as in Example 1 with the PEG lipids indicated in Table 58 in a lipid composition of 50/10/38.5/1.5, expressed as the molar ratio of ionizable lipid A/cholesterol/DSPC/PEG, respectively. Immediately prior to exposure to T cells at doses indicated in Table 58, LNPs were preincubated at 37 C for about 5-15 minutes with 20 ug/mL ApoE3 (Peprotech, Cat #350-02) in T cell base media with 5% human serum. T cells were plated as indicated in Example 20.1 prior to LNP treatment. Following LNP treatment, complete T cell media was replaced every 48h at each respective time points and were collected for Flow Cytomtery analyses for CD3 surface expression 7 days post LNP treatment for cells treated at 0 hours and 4 days post second LNP treatment for cells treated at 72 hours.

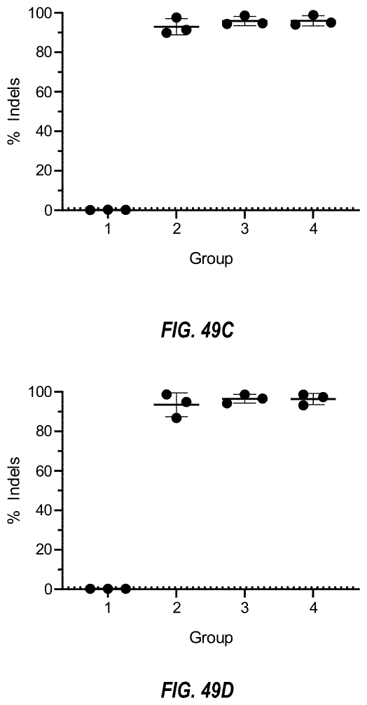

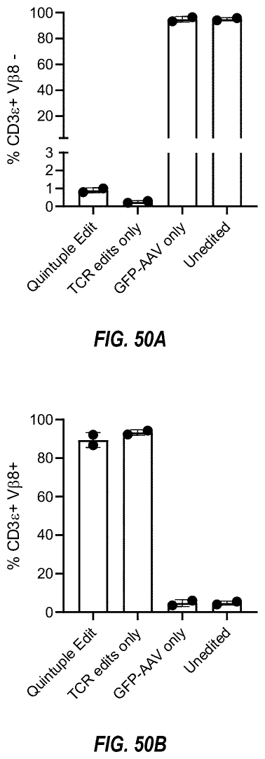

Table 58 and FIGS. 52A and 52B show the percentage of CD3 negative cells following non-activated T cell treatment as described. Cells treated with co-formulated LNPs exhibited higher percentage of CD3 negative cells than cells treated first with mRNA-only LNPs. Higher CD3 negative percentage was seen for co-formulated cargo with both PEG-2kDMG or PEG Lipid H lipid formulations. Dose dependent editing was observed when sgRNA-only cargo was delivered 0 hours or 72 hours after mRNA-only cargo. Similar dose-dependent editing response was observed with both first lipid formulations when the second gRNA-only LNP was added 24 hours or 48 hours after the initial LNP treatment.

TABLE 58
Percent CD3 negative cells after non-activated T cell treatment
with co-formulated or mRNA-only first LNPs at 0 hours and
gRNA-only second LNPs at 0 hours or 72 hours.

| | First LNP: | | | | | | | | |
|---|---|---|---|---|---|---|---|---|---|
| | | Co-formulated | | | | | | | |
| | Cargo | mRNA + gRNA | | | | mRNA ONLY | | | |
| | PEG | Lipid H | | 2k-DMG | | Lipid H | | 2k-DMG | |
| Sec- | LNP | Bio | Bio | Bio | Bio | Bio | Bio | Bio | Bio |
| ond | (ug/ | rep | rep | rep | rep | rep | rep | rep | rep |
| LNP | mL) | 1 | 2 | 1 | 2 | 1 | 2 | 1 | 2 |
| 0 h | 20.00 | 60.0 | 54.8 | 67.6 | 68.4 | 58.2 | 61.3 | 58.4 | 59.3 |
| | 10.00 | 67.2 | 58.4 | 61.6 | 62.8 | 59.2 | 54.1 | 57.5 | 55.0 |
| | 5.00 | 62.2 | 56.8 | 64.9 | 59.9 | 54.3 | 48.1 | 45.3 | 52.6 |
| | 2.50 | 58.8 | 50.4 | 45.9 | 53.0 | 36.8 | 42.7 | 36.8 | 37.8 |
| | 1.25 | 42.5 | 32.7 | 32.1 | 33.6 | 13.3 | 14.3 | 12.5 | 18.6 |
| | 0.63 | 16.8 | 11.4 | 9.1 | 11.7 | 3.2 | 4.1 | 3.3 | 5.4 |
| | 0.31 | 3.5 | 3.1 | 3.2 | 3.4 | 2.9 | 3.5 | 3.2 | 2.1 |
| 72 h | 0.00 | 1.2 | 1.9 | 1.8 | 1.5 | 1.6 | 1.4 | 2.2 | 1.6 |
| | 20.00 | 64.9 | 63.9 | 76.8 | 70.1 | 50.3 | 51.9 | 42.9 | 52.9 |
| | 10.00 | 70.7 | 69.4 | 71.6 | 76.7 | 57.4 | 47.6 | 51.5 | 46.3 |
| | 5.00 | 67.4 | 63.3 | 71.4 | 73.1 | 43.3 | 43.9 | 39.9 | 33.0 |
| | 2.50 | 75.4 | 70.4 | 65.5 | 62.6 | 20.3 | 27.8 | 18.7 | 22.6 |
| | 1.25 | 67.5 | 50.3 | 39.5 | 38.6 | 10.6 | 11.7 | 12.2 | 8.7 |
| | 0.63 | 32.5 | 26.6 | 14.2 | 16.9 | 6.6 | 7.4 | 5.0 | 9.7 |
| | 0.31 | 11.4 | 8.3 | 6.5 | 5.9 | 4.8 | 4.8 | 3.2 | 7.3 |
| | 0.00 | 7.4 | 7.1 | 5.6 | 7.5 | 8.9 | 4.7 | 4.0 | 5.7 |

### Example 21. Lipid a Composition Screens in T Cells

To evaluate editing efficacy, T cells were treated with LNP compositions with varied molar ratios of lipid components encapsulating Cas9 mRNA and a sgRNA targeting the TRAC gene. Editing was assayed by an increase in the percentage of CD3 negative cells following editing. The T cell receptor alpha chain encoded by TRAC is required for T cell receptor/CD3 complex assembly and translocation to the cell surface. Accordingly, disruption of the TRAC gene by genome editing leads to a loss of CD3 protein on the cell surface of T cells.

Healthy human donor apheresis was obtained commercially (Hemacare). T cells were isolated by negative selection using the EasySep Human T cell Isolation Kit (Stem Cell Technology, Cat. 17951) or by CD4/CD8 positive selection using the StraightFrom® Leukopak® CD4/CD8 MicroBeads (Miltenyi, Catalog, 130-122-352) on the MultiMACS Cell24 Separator Plus instrument following manufacturers instruction. T cells were cryopreserved in Cryostor CS10 freezing media (Cat., 07930) for future use.

Upon thaw, T cells to be activated were plated in complete T cell growth media composed of CTS OpTmizer Base Media (CTS OpTmizer Media (Gibco, A3705001) supplemented with 1× GlutaMAX, 10 mM HEPES buffer (10 mM), and 1% Penicillin/Streptomycin) further supplemented 200 U/ml IL-2, 5 ng/ml IL7 and 5 ng/ml IL-15 and 2.5% human serum (Gemini, 100-512). After overnight rest, T cells at a density of 1e6/mL were activated with T cell TransAct Reagent (1:100 dilution, Miltenyi) if indicated and incubated for 48 hours. Post incubation, activated cells at a density of 0.5e6 cells/mL were used for editing applications.

The same process was used for non-activated T cells with the following exceptions. Upon thaw, non-Activated T cells were cultured in the CTS complete growth with 5% Human Serum for 24 hrs without activation. T cells were then plated at a cell density of 1e6/mL in 100 uL of complete T cell growth media for editing applications.

T cells were transfected with LNPs formulated as described in Example 1 with lipid compositions as indicated in Table 59, expressed as the molar ratio of ionizable Lipid A/cholesterol/DSPC/PEG. LNPs delivered mRNA encoding Cas9 (SEQ ID NO. 6) and sgRNA (G013006) (SEQ ID NO: 708) targeting TRAC at doses indicated tin Table 59. The cargo ratio of sgRNA to Cas9 mRNA was 1:2 by weight. N:P ratio was about 6 unless otherwise indicated.

The LNP dose response curves (DRCs) transfection was performed on the Hamilton Microlab STAR liquid handling system. The liquid handler was provided with the following: (a) 4× the desired highest LNP dose in the top row of a 96-deep well plate, (b) ApoE3 diluted in media at 20 ug/mL, (c) complete T cell growth media composed of CTS OpTmizer Base Media (CTS OpTmizer Media (Gibco, A3705001) supplemented with 1× GlutaMAX, 10 mM HEPES buffer (10 mM), and 1% Penicillin/Streptomycin) further supplemented 200 IU/ml IL-2, 5 ng/ml IL7 and 5 ng/ml IL-15 and 2.5% human serum (Gemini, 100-512). and (d) T cells plated at 1 e6/m1 density in 100 uL in 96-well flat bottom tissue culture plates. The liquid handler first performed an 8-point two-fold serial dilution of the LNPs starting from the 4× LNP dose in the deep well plate. After this, equal volume of ApoE3 media was added to each well resulting in a 1:1 dilution of both LNP and ApoE3. Subsequently 100 uL of the LNP-ApoE mix was added to each T cell plate. The final concentration of LNPs at the top dose was set to be 5 ug/mL. Final concentrations of ApoE3 at 5 ug/mL and T cells were at a final density of 0.5e6 cells/mL. Plates were incubated at 37 C with 5% CO2 for 7 days and then harvested for flow cytometry analysis.

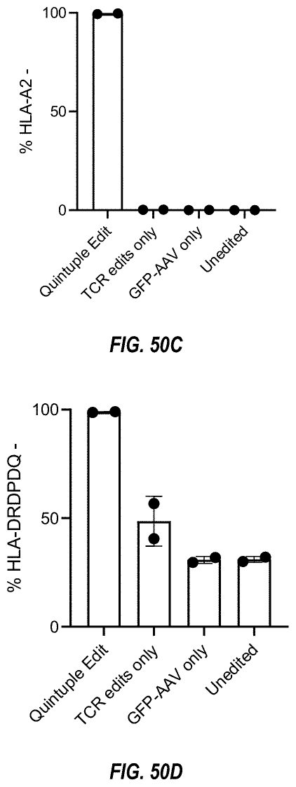

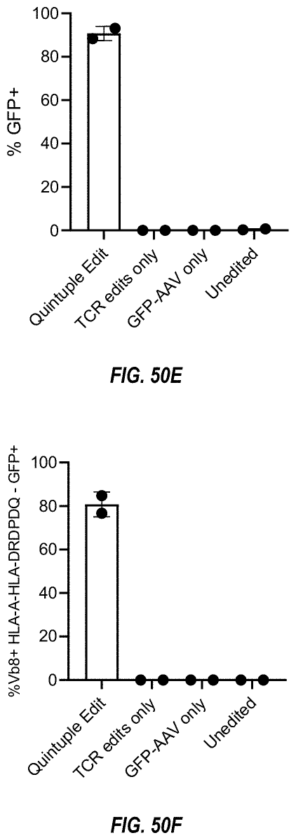

To assay cell surface proteins by flow cytometry, T cells were incubated with antibodies targeting CD3 (Biolegend, Cat. 300441), CD4 (Biolegend, Cat. 300538), and CD8a (Biolegend, Cat. 301049). T cells were subsequently processed on a Cytoflex instrument (Beckman Coulter). Data analysis was performed using FlowJo software package (v.10.6.1 or v.10.7.1). Briefly, T cells were gated on lymphocytes followed by single cells. These single cells were gated on CD4+/CD8+ status from which CD8+/CD3− cells were selected. Table 59 and FIG. 53A show CD3 negative cells after activated T cell treatment with indicated LNP compositions. Table 60 and FIG. 53B show CD3 negative cells after non-activated T cell treatment with indicated LNP compositions.

TABLE 59
Mean percent CD3 negative cells following activated T cell
treatment with indicated LNP formulations.

| LNP | LNP | | | | |
|---|---|---|---|---|---|
| Composition | (ug/mL) | Mean | SD | N | EC50 |
| 50/10/38.5/1.5 | 5.00 | 95.3 | 0.5 | 2 | 1.55 |
| | 2.50 | 81.7 | 0.8 | 2 | |
| | 1.25 | 31.8 | 3.1 | 2 | |
| | 0.63 | 5.8 | 0.6 | 2 | |
| | 0.31 | 1.4 | 0.0 | 2 | |
| | 0.16 | 0.6 | 0.0 | 2 | |
| | 0.08 | 0.4 | 0.0 | 2 | |
| | 0.04 | 0.3 | 0.1 | 2 | |
| 50/5/43.5/1.5 | 5.00 | 88.8 | 0.2 | 2 | 1.41 |
| | 2.50 | 77.8 | 0.7 | 2 | |
| | 1.25 | 37.4 | 1.6 | 2 | |
| | 0.63 | 8.2 | 0.8 | 2 | |
| | 0.31 | 2.9 | 0.7 | 2 | |
| | 0.16 | 0.6 | 0.0 | 2 | |
| | 0.08 | 0.3 | 0.0 | 2 | |
| | 0.04 | 0.3 | 0.0 | 2 | |
| 45/15/38.5/1.5 | 5.00 | 97.1 | 0.2 | 2 | 1.41 |
| | 2.50 | 87.6 | 0.8 | 2 | |
| | 1.25 | 38.6 | 0.6 | 2 | |
| | 0.63 | 6.6 | 0.7 | 2 | |
| | 0.31 | 1.3 | 0.2 | 2 | |
| | 0.16 | 0.4 | 0.1 | 2 | |
| | 0.08 | 0.3 | 0.1 | 2 | |
| | 0.04 | 0.2 | 0.1 | 2 | |
| 45/5/48.5/1.5 | 5.00 | 90.2 | 1.1 | 2 | 1.42 |
| | 2.50 | 76.7 | 0.0 | 2 | |
| | 1.25 | 38.5 | 0.9 | 2 | |
| | 0.63 | 8.5 | 0.5 | 2 | |
| | 0.31 | 1.5 | 0.3 | 2 | |
| | 0.16 | 0.5 | 0.0 | 2 | |
| | 0.08 | 0.5 | 0.1 | 2 | |
| | 0.04 | 0.5 | 0.2 | 2 | |
| 40/10/48.5/1.5 | 5.00 | 94.0 | 1.7 | 2 | 0.93 |
| | 2.50 | 89.7 | 1.2 | 2 | |
| | 1.25 | 66.5 | 0.8 | 2 | |
| | 0.63 | 24.5 | 0.2 | 2 | |
| | 0.31 | 6.8 | 0.2 | 2 | |
| | 0.16 | 2.2 | 0.2 | 2 | |
| | 0.08 | 0.8 | 0.1 | 2 | |
| | 0.04 | 0.6 | 0.0 | 2 | |
| 30/10/58.5/1.5 | 5.00 | 4.7 | 0.2 | 2 | 6.43 |
| | 2.50 | 3.1 | 0.8 | 2 | |
| | 1.25 | 1.6 | 0.3 | 2 | |
| | 0.63 | 1.1 | 0.0 | 2 | |
| | 0.31 | 0.7 | 0.1 | 2 | |
| | 0.16 | 0.6 | 0.0 | 2 | |
| | 0.08 | 0.3 | 0.0 | 2 | |
| | 0.04 | 0.3 | 0.0 | 2 | |
| 30/5/63.5/1.5 | 5.00 | 52.0 | 1.6 | 2 | 3.19 |
| | 2.50 | 34.3 | 2.7 | 2 | |
| | 1.25 | 26.0 | 0.4 | 2 | |
| | 0.63 | 15.6 | 0.2 | 2 | |
| | 0.31 | 7.3 | 0.4 | 2 | |
| | 0.16 | 2.9 | 0.2 | 2 | |
| | 0.08 | 1.3 | 0.1 | 2 | |
| | 0.04 | 0.4 | 0.1 | 2 | |
| 55/5/38.5/1.5 | 5.00 | 67.0 | 2.0 | 2 | 2.68 |
| | 2.50 | 36.7 | 0.5 | 2 | |
| | 1.25 | 9.8 | 1.1 | 2 | |
| | 0.63 | 2.6 | 0.1 | 2 | |
| | 0.31 | 0.6 | 0.2 | 2 | |
| | 0.16 | 0.4 | 0.1 | 2 | |
| | 0.08 | 0.1 | 0.1 | 2 | |
| | 0.04 | 0.3 | 0.1 | 2 | |
| 55/10/33.5/1.5 | 5.00 | 89.2 | 0.6 | 2 | 2.30 |
| | 2.50 | 54.6 | 1.1 | 2 | |
| | 1.25 | 7.3 | 0.2 | 2 | |
| | 0.63 | 1.4 | 0.1 | 2 | |
| | 0.31 | 0.5 | 0.2 | 2 | |
| | 0.16 | 0.3 | 0.1 | 2 | |
| | 0.08 | 0.2 | 0.1 | 2 | |
| | 0.04 | 0.2 | 0.1 | 2 | |
| 65/5/28.5/1.5 | 5.00 | 0.2 | 0.1 | 2 | 0.50 |
| | 2.50 | 0.2 | 0.0 | 2 | |
| | 1.25 | 0.3 | 0.0 | 2 | |
| | 0.63 | 0.4 | 0.1 | 2 | |
| | 0.31 | 0.2 | 0.0 | 2 | |
| | 0.16 | 0.1 | 0.1 | 2 | |
| | 0.08 | 0.3 | 0.0 | 2 | |
| | 0.04 | 0.3 | 0.1 | 2 | |
| 50/10/38.5/1.5 | 5.00 | 95.6 | 0.1 | 2 | 1.17 |
| (N/P: 5.0) | 2.50 | 90.0 | 1.0 | 2 | |
| | 1.25 | 53.4 | 1.3 | 2 | |
| | 0.63 | 16.4 | 1.7 | 2 | |
| | 0.31 | 4.5 | 0.2 | 2 | |
| | 0.16 | 1.8 | 0.1 | 2 | |
| | 0.08 | 0.5 | 0.0 | 2 | |
| | 0.04 | 0.6 | 0.1 | 2 | |
| 50/10/38.5/1.5 | 5.00 | 97.7 | 0.1 | 2 | 0.90 |
| (N/P: 7.0) | 2.50 | 93.5 | 0.2 | 2 | |
| | 1.25 | 73.0 | 0.9 | 2 | |
| | 0.63 | 24.0 | 0.2 | 2 | |
| | 0.31 | 5.5 | 0.1 | 2 | |
| | 0.16 | 1.5 | 0.5 | 2 | |
| | 0.08 | 0.9 | 0.3 | 2 | |
| | 0.04 | 0.6 | 0.1 | 2 | |

TABLE 60
Mean percent CD3 negative cells following non-activated T cell
treatment with indicated LNP formulations.

| | LNP | Mean | | | |
|---|---|---|---|---|---|
| | Dose | % | | | |
| LNP Composition | (ug/mL) | CD3- | SD | N | EC50 |
| 50/10/38.5/1.5 | 5.00 | 69.7 | 0.9 | 2 | 1.21 |
| | 2.50 | 65.1 | 1.3 | 2 | |
| | 1.25 | 41.3 | 8.9 | 2 | |
| | 0.63 | 11.1 | 4.0 | 2 | |
| | 0.31 | 10.2 | 0.5 | 2 | |
| | 0.16 | 8.1 | 1.5 | 2 | |
| | 0.08 | 9.9 | 2.0 | 2 | |
| | 0.04 | 7.2 | 2.5 | 2 | |
| 50/5/43.5/1.5 | 5.00 | 47.6 | 3.6 | 2 | 4.71 |
| | 2.50 | 30.0 | 5.0 | 2 | |
| | 1.25 | 20.1 | 2.9 | 2 | |
| | 0.63 | 11.4 | 2.3 | 2 | |
| | 0.31 | 9.8 | 1.3 | 2 | |
| | 0.16 | 9.1 | 0.0 | 2 | |
| | 0.08 | 9.3 | 0.8 | 2 | |
| | 0.04 | 7.9 | 3.6 | 2 | |
| 45/15/38.5/1.5 | 5.00 | 83.1 | 1.5 | 2 | 1.36 |
| | 2.50 | 73.0 | 3.1 | 2 | |
| | 1.25 | 43.4 | 6.8 | 2 | |
| | 0.63 | 19.4 | 3.4 | 2 | |
| | 0.31 | 11.7 | 2.4 | 2 | |
| | 0.16 | 8.9 | 1.3 | 2 | |
| | 0.08 | 9.0 | 1.6 | 2 | |
| | 0.04 | 9.5 | 2.9 | 2 | |
| 45/5/48.5/1.5 | 5.00 | 49.0 | 1.7 | 2 | 2.99 |
| | 2.50 | 33.7 | 6.3 | 2 | |
| | 1.25 | 25.8 | 2.3 | 2 | |
| | 0.63 | 14.7 | 1.0 | 2 | |
| | 0.31 | 9.8 | 0.7 | 2 | |
| | 0.16 | 11.2 | 3.9 | 2 | |
| | 0.08 | 10.2 | 1.9 | 2 | |
| | 0.04 | 9.5 | 2.5 | 2 | |
| 40/10/48.5/1.5 | 5.00 | 60.5 | 3.2 | 2 | 0.66 |
| | 2.50 | 61.6 | 4.4 | 2 | |
| | 1.25 | 57.9 | 6.7 | 2 | |
| | 0.63 | 34.0 | 7.9 | 2 | |
| | 0.31 | 12.1 | 0.8 | 2 | |
| | 0.16 | 10.4 | 1.2 | 2 | |
| | 0.08 | 13.7 | 0.4 | 2 | |
| | 0.04 | 10.4 | 4.7 | 2 | |
| 30/10/58.5/1.5 | 5.00 | 11.0 | 0.1 | 2 | Not |
| | 2.50 | 11.3 | 2.9 | 2 | available |
| | 1.25 | 8.0 | 0.5 | 2 | |
| | 0.63 | 11.3 | 0.5 | 2 | |
| | 0.31 | 10.3 | 0.7 | 2 | |
| | 0.16 | 9.3 | 1.3 | 2 | |
| | 0.08 | 10.8 | 0.8 | 2 | |
| | 0.04 | 10.7 | 5.1 | 2 | |
| 30/5/63.5/1.5 | 5.00 | 26.7 | 1.0 | 2 | 0.37 |
| | 2.50 | 18.9 | 0.1 | 2 | |
| | 1.25 | 19.6 | 1.5 | 2 | |
| | 0.63 | 23.0 | 1.2 | 2 | |
| | 0.31 | 15.4 | 0.5 | 2 | |
| | 0.16 | 11.8 | 3.5 | 2 | |
| | 0.08 | 12.5 | 0.3 | 2 | |
| | 0.04 | 9.9 | 3.4 | 2 | |
| 55/5/38.5/1.5 | 5.00 | 24.6 | 1.0 | 2 | 2.50 |
| | 2.50 | 18.0 | 2.4 | 2 | |
| | 1.25 | 10.1 | 2.2 | 2 | |
| | 0.63 | 12.4 | 3.2 | 2 | |
| | 0.31 | 9.5 | 3.2 | 2 | |
| | 0.16 | 10.4 | 1.3 | 2 | |
| | 0.08 | 11.4 | 1.5 | 2 | |
| | 0.04 | 12.4 | 2.7 | 2 | |
| 55/10/33.5/1.5 | 5.00 | 61.0 | 3.7 | 2 | 1.57 |
| | 2.50 | 53.6 | 6.5 | 2 | |
| | 1.25 | 26.5 | 7.3 | 2 | |
| | 0.63 | 12.4 | 2.4 | 2 | |
| | 0.31 | 13.1 | 5.7 | 2 | |
| | 0.16 | 9.9 | 2.9 | 2 | |
| | 0.08 | 0.4 | 1.5 | 2 | |
| | 0.04 | 10.1 | 3.0 | 2 | |
| 65/5/28.5/1.5 | 5.00 | 14.7 | 0.8 | 2 | 6.05 |
| | 2.50 | 8.6 | 1.6 | 2 | |
| | 1.25 | 10.9 | 2.9 | 2 | |
| | 0.63 | 10.7 | 1.1 | 2 | |
| | 0.31 | 12.0 | 3.8 | 2 | |
| | 0.16 | 10.4 | 4.8 | 2 | |
| | 0.08 | 10.6 | 0.6 | 2 | |
| | 0.04 | 8.9 | 2.2 | 2 | |
| 50/10/38.5/1.5 | 5.00 | 74.4 | 2.3 | 2 | 1.30 |
| (N/P: 5.0) | 2.50 | 69.6 | 7.9 | 2 | |
| | 1.25 | 40.5 | 7.2 | 2 | |
| | 0.63 | 19.5 | 0.3 | 2 | |
| | 0.31 | 8.9 | 3.2 | 2 | |
| | 0.16 | 9.3 | 1.1 | 2 | |
| | 0.08 | 9.9 | 0.2 | 2 | |
| | 0.04 | 14.3 | 14.3 | 2 | |
| 50/10/38.5/1.5 | 5.00 | 76.0 | 0.5 | 2 | 0.77 |
| (N/P: 7.0) | 2.50 | 68.4 | 2.1 | 2 | |
| | 1.25 | 65.4 | 3.8 | 2 | |
| | 0.63 | 27.9 | 4.9 | 2 | |
| | 0.31 | 12.1 | 1.9 | 2 | |
| | 0.16 | 9.3 | 0.5 | 2 | |
| | 0.08 | 10.5 | 2.2 | 2 | |
| | 0.04 | 0.0 | 0.0 | 2 | |

### Example 22. Cargo Ratio Evaluation of Selected LNP Compositions

To evaluate editing efficacy, T cells were treated with LNP compositions with varying ratios of Cas9 mRNA and sgRNA targeting the TRAC gene. Editing was assayed by an increase in the percentage of CD3 negative cells following editing. The T cell receptor alpha chain encoded by TRAC is required for T cell receptor/CD3 complex assembly and translocation to the cell surface. Accordingly, disruption of the TRAC gene by genome editing leads to a loss of CD3 protein on the cell surface of T cells.

### Example 22.1. Cargo Ratio Evaluation in Activated T Cells

LNP compositions were tested in vitro to evaluate the effect of varied cargo ratios on the editing efficiency of LNPs in CD3+ T cells. LNPs delivered mRNA encoding Cas9 (SEQ ID NO: 7) and the sgRNA targeting human TRAC (G013006) (SEQ ID NO: 708). LNPs were formulated as described in Example 1 at lipid compositions of 50/10/38/1.5 or 35/15/47.5/2.5 expressed as the molar ratio of ionizable Lipid A/cholesterol/DSPC/PEG, respectively. The cargo ratio of sgRNA to Cas9 mRNA was 1:2, 1:1, 2:1, or 4:1 by weight.

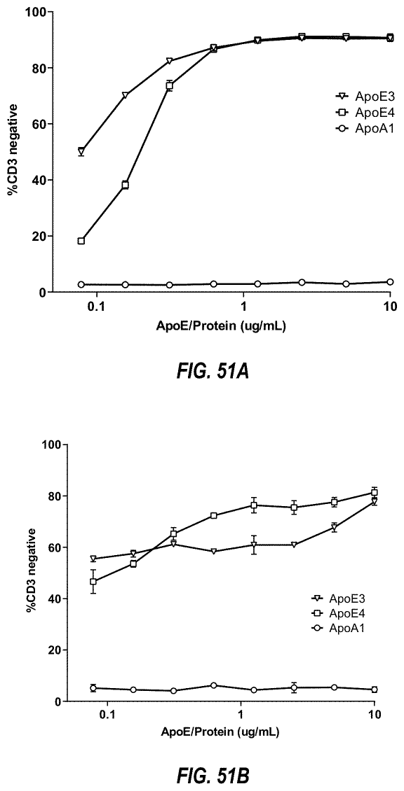

T cells were cultured, prepared, and activated as described in Example 21. Forty-eight hours post activation, the activated T cells were transfected with pre-incubated LNP as described in Example 21. Seven days post transfection, T cells were phenotyped by flow cytometry analysis as described in Example 21. Results are shown in Table 61 and FIG. 54. Dose dependent editing was seen in activated T cells treated with LNPs of both lipid formulations.

TABLE 61
Percent CD3 negative cells following
activated T cell treatment
with LNPs with varied cargo ratios

| Lipid | Cargo | LNP | | | | |
|---|---|---|---|---|---|---|
| Composition | ratio | (ug/mL) | Mean | SD | N | EC50 |
| 50/10/38.5/1.5 | 1:2 | 5 | 95.4 | 0.6 | 2 | 0.98 |
| | | 2.5 | 93.0 | 0.9 | 2 | |
| | | 1.25 | 68.4 | 2.9 | 2 | |
| | | 0.63 | 17.1 | 2.2 | 2 | |
| | | 0.31 | 4.7 | 0.7 | 2 | |
| | | 0.16 | 1.3 | 0.1 | 2 | |
| | | 0.08 | 0.9 | 0.0 | 2 | |
| | | 0.04 | 0.3 | 0.0 | 2 | |
| | 1:1 | 5 | 95.7 | 0.3 | 2 | 0.89 |
| | | 2.5 | 92.8 | 1.0 | 2 | |
| | | 1.25 | 74.4 | 1.0 | 2 | |
| | | 0.63 | 22.1 | 2.6 | 2 | |
| | | 0.31 | 5.4 | 0.3 | 2 | |
| | | 0.16 | 1.8 | 0.4 | 2 | |
| | | 0.08 | 0.8 | 0.1 | 2 | |
| | | 0.04 | 0.4 | 0.1 | 2 | |
| | 2:1 | 5 | 95.4 | 0.6 | 2 | 0.89 |
| | | 2.5 | 92.5 | 1.0 | 2 | |
| | | 1.25 | 75.2 | 2.4 | 2 | |
| | | 0.63 | 21.5 | 2.7 | 2 | |
| | | 0.31 | 5.0 | 0.1 | 2 | |
| | | 0.16 | 1.8 | 0.5 | 2 | |
| | | 0.08 | 0.8 | 0.2 | 2 | |
| | | 0.04 | 0.6 | 0.1 | 2 | |
| | 4:1 | 5 | 94.3 | 0.4 | 2 | 1.18 |
| | | 2.5 | 89.2 | 0.2 | 2 | |
| | | 1.25 | 52.4 | 0.2 | 2 | |
| | | 0.63 | 11.3 | 1.2 | 2 | |
| | | 0.31 | 2.4 | 0.2 | 2 | |
| | | 0.16 | 0.7 | 0.1 | 2 | |
| | | 0.08 | 0.7 | 0.2 | 2 | |
| | | 0.04 | 0.4 | 0.1 | 2 | |
| 35/15/47.5/2.5 | 1:2 | 5 | 97.3 | 0.5 | 2 | 0.29 |
| | | 2.5 | 96.3 | 0.3 | 2 | |
| | | 1.25 | 93.8 | 0.6 | 2 | |
| | | 0.63 | 84.7 | 0.8 | 2 | |
| | | 0.31 | 53.8 | 2.3 | 2 | |
| | | 0.16 | 22.2 | 0.4 | 2 | |
| | | 0.08 | 8.6 | 0.2 | 2 | |
| | | 0.04 | 2.6 | 0.0 | 2 | |
| | 1:1 | 5 | 97.2 | 0.8 | 2 | 0.19 |
| | | 2.5 | 97.1 | 1.0 | 2 | |
| | | 1.25 | 96.9 | 0.2 | 2 | |
| | | 0.63 | 93.7 | 0.4 | 2 | |
| | | 0.31 | 76.8 | 2.9 | 2 | |
| | | 0.16 | 42.1 | 0.7 | 2 | |
| | | 0.08 | 15.9 | 0.7 | 2 | |
| | | 0.04 | 5.8 | 0.1 | 2 | |
| | 2:1 | 5 | 97.2 | 0.7 | 2 | 0.24 |
| | | 2.5 | 97.1 | 0.4 | 2 | |
| | | 1.25 | 96.0 | 0.4 | 2 | |
| | | 0.63 | 92.4 | 0.8 | 2 | |
| | | 0.31 | 67.1 | 1.2 | 2 | |
| | | 0.16 | 25.4 | 1.3 | 2 | |
| | | 0.08 | 7.3 | 0.3 | 2 | |
| | | 0.04 | 2.4 | 0.1 | 2 | |
| | 4:1 | 5 | 97.6 | 0.3 | 2 | 0.21 |
| | | 2.5 | 97.3 | 0.5 | 2 | |
| | | 1.25 | 96.2 | 0.6 | 2 | |
| | | 0.63 | 94.9 | 0.3 | 2 | |
| | | 0.31 | 73.8 | 1.6 | 2 | |
| | | 0.16 | 33.1 | 0.9 | 2 | |
| | | 0.08 | 9.5 | 0.1 | 2 | |
| | | 0.04 | 3.3 | 0.5 | 2 | |

### Example 22.2. Cargo Ratio Evaluation in Non-Activated T Cells

The effect of varied cargo ratios on the editing efficiency of LNPs was tested in non-activated CD3+ T cells. The selected LNP compositions described in Example 22.1 were used in this study. T cells were obtained from two donors and samples from each donor were prepared as described in Example 21. Non-activated T cells were cultured for twenty-four hours before they were transfected with pre-incubated LNP as described in Example 21. Seven days post transfection, T cells were phenotyped by flow cytometry analysis as described in Example 21.

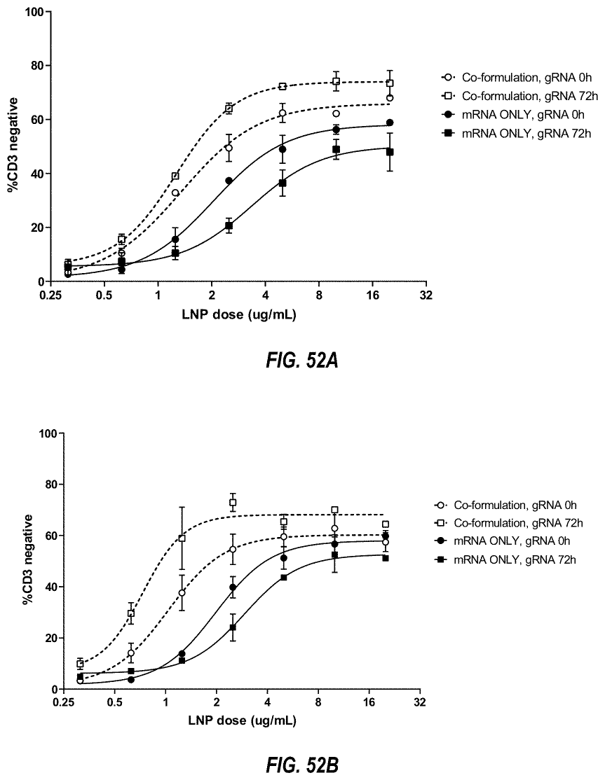

Edited T cells were phenotyped by flow cytometry as described in Example 21 to evaluate the impact of each cargo ratio on the editing efficiency of the LNP compositions. Results are shown in Table 62 and FIGS. 55A-B. Dose dependent editing was seen in non-activated T cells treated with LNPs of both lipid formulations.

TABLE 62
Percent CD3 negative cells following non-activated T cell
treatment with LNPs with varied cargo ratios

| Lipid | Car- | LNP | | | | | | | | |
|---|---|---|---|---|---|---|---|---|---|---|
| Compo- | go | (ug/ | Donor 1 | | | | Donor 2 | | | |
| sition | ratio | mL) | Mean | SD | N | EC50 | Mean | SD | N | EC50 |
| 50/10/ | 1:2 | 5 | 61.3 | 0.1 | 2 | 1.99 | 86.6 | 0.0 | 2 | 1.63 |
| 38.5/ | | 2.5 | 44.1 | 0.1 | 2 | | 70.2 | 0.0 | 2 | |
| 1.5 | | 1.25 | 13.3 | 0.0 | 2 | | 36.7 | 0.0 | 2 | |
| | | 0.63 | 4.9 | 0.0 | 2 | | 11.5 | 0.0 | 2 | |
| | | 0.31 | 2.1 | 0.0 | 2 | | 12.3 | 0.1 | 2 | |
| | | 0.16 | 1.5 | 0.0 | 2 | | 13.5 | 0.0 | 2 | |
| | | 0.08 | 1.5 | 0.0 | 2 | | 11.4 | 0.0 | 2 | |
| | | 0.04 | 1.6 | 0.0 | 2 | | 7.8 | 0.0 | 2 | |
| | 1:1 | 5 | 65.5 | 0.0 | 2 | 1.86 | 84.1 | 0.0 | 2 | 1.70 |
| | | 2.5 | 50.7 | 0.1 | 2 | | 70.6 | 0.0 | 2 | |
| | | 1.25 | 14.9 | 0.0 | 2 | | 29.2 | 0.0 | 2 | |
| | | 0.63 | 5.2 | 0.0 | 2 | | 14.9 | 0.1 | 2 | |
| | | 0.31 | 1.9 | 0.0 | 2 | | 12.5 | 0.0 | 2 | |
| | | 0.16 | 2.1 | 0.0 | 2 | | 8.4 | 0.0 | 2 | |
| | | 0.08 | 2.3 | 0.0 | 2 | | 8.2 | 0.0 | 2 | |
| | | 0.04 | 1.9 | 0.0 | 2 | | 8.2 | 0.0 | 2 | |
| | 2:1 | 5 | 59.3 | 0.0 | 2 | 1.94 | 79.8 | 0.0 | 2 | 1.82 |
| | | 2.5 | 43.5 | 0.1 | 2 | | 63.1 | 0.1 | 2 | |
| | | 1.25 | 13.9 | 0.0 | 2 | | 25.6 | 0.0 | 2 | |
| | | 0.63 | 2.4 | 0.0 | 2 | | 9.8 | 0.0 | 2 | |
| | | 0.31 | 2.4 | 0.0 | 2 | | 7.7 | 0.0 | 2 | |
| | | 0.16 | 2.5 | 0.0 | 2 | | 9.9 | 0.0 | 2 | |
| | | 0.08 | 2.1 | 0.0 | 2 | | 10.2 | 0.0 | 2 | |
| | | 0.04 | 3.1 | 0.0 | 2 | | 9.7 | 0.0 | 2 | |
| | 4:1 | 5 | 42.6 | 0.1 | 2 | 2.96 | 64.1 | 0.0 | 2 | 2.62 |
| | | 2.5 | 18.1 | 0.0 | 2 | | 38.6 | 0.0 | 2 | |
| | | 1.25 | 4.0 | 0.0 | 2 | | 16.0 | 0.1 | 2 | |
| | | 0.63 | 2.0 | 0.0 | 2 | | 9.6 | 0.0 | 2 | |
| | | 0.31 | 1.7 | 0.0 | 2 | | 9.1 | 0.0 | 2 | |
| | | 0.16 | 2.7 | 0.0 | 2 | | 7.9 | 0.0 | 2 | |
| | | 0.08 | 2.7 | 0.0 | 2 | | 10.6 | 0.0 | 2 | |
| | | 0.04 | 1.9 | 0.0 | 2 | | 11.0 | 0.0 | 2 | |
| 35/15/ | 1:2 | 5 | 71.4 | 0.0 | 2 | 0.65 | 91.3 | 0.0 | 2 | 0.51 |
| 47.5/ | | 2.5 | 69.6 | 0.0 | 2 | | 86.4 | 0.0 | 2 | |
| 2.5 | | 1.25 | 61.8 | 0.0 | 2 | | 80.5 | 0.0 | 2 | |
| | | 0.63 | 34.7 | 0.0 | 2 | | 60.6 | 0.0 | 2 | |
| | | 0.31 | 10.8 | 0.0 | 2 | | 25.5 | 0.0 | 2 | |
| | | 0.16 | 3.5 | 0.0 | 2 | | 11.6 | 0.0 | 2 | |
| | | 0.08 | 2.0 | 0.0 | 2 | | 12.6 | 0.0 | 2 | |
| | | 0.04 | 2.3 | 0.0 | 2 | | 7.4 | 0.0 | 2 | |
| | 1:1 | 5 | 76.5 | 0.1 | 2 | 0.57 | 94.1 | 0.0 | 2 | 0.39 |
| | | 2.5 | 80.0 | 0.0 | 2 | | 91.4 | 0.0 | 2 | |
| | | 1.25 | 70.9 | 0.0 | 2 | | 90.7 | 0.0 | 2 | |
| | | 0.63 | 45.8 | 0.0 | 2 | | 72.4 | 0.0 | 2 | |
| | | 0.31 | 15.9 | 0.0 | 2 | | 40.0 | 0.0 | 2 | |
| | | 0.16 | 5.3 | 0.0 | 2 | | 17.9 | 0.0 | 2 | |
| | | 0.08 | 3.9 | 0.0 | 2 | | 9.7 | 0.0 | 2 | |
| | | 0.04 | 2.8 | 0.0 | 2 | | 8.8 | 0.0 | 2 | |
| | 2:1 | 5 | 69.2 | 0.1 | 2 | 0.66 | 92.6 | 0.0 | 2 | 0.61 |
| | | 2.5 | 66.5 | 0.0 | 2 | | 84.3 | 0.0 | 2 | |
| | | 1.25 | 53.8 | 0.1 | 2 | | 82.2 | 0.0 | 2 | |
| | | 0.63 | 36.5 | 0.1 | 2 | | 51.7 | 0.0 | 2 | |
| | | 0.31 | 7.2 | 0.0 | 2 | | 22.3 | 0.1 | 2 | |
| | | 0.16 | 3.0 | 0.0 | 2 | | 17.7 | 0.1 | 2 | |
| | | 0.08 | 2.5 | 0.0 | 2 | | 9.8 | 0.0 | 2 | |
| | | 0.04 | 1.9 | 0.0 | 2 | | 11.5 | 0.0 | 2 | |
| | 4:1 | 5 | 66.1 | 0.0 | 2 | 0.53 | 89.4 | 0.0 | 2 | 0.50 |
| | | 2.5 | 65.0 | 0.1 | 2 | | 87.1 | 0.0 | 2 | |
| | | 1.25 | 58.8 | 0.0 | 2 | | 79.9 | 0.0 | 2 | |
| | | 0.63 | 44.4 | 0.1 | 2 | | 64.5 | 0.0 | 2 | |
| | | 0.31 | 7.8 | 0.0 | 2 | | 22.8 | 0.1 | 2 | |
| | | 0.16 | 2.9 | 0.0 | 2 | | 9.7 | 0.0 | 2 | |
| | | 0.08 | 2.4 | 0.0 | 2 | | 14.5 | 0.1 | 2 | |
| | | 0.04 | 2.9 | 0.0 | 2 | | 10.5 | 0.0 | 2 | |

### Example 23. Editing in B Cell Using Lipid Nanoparticles

### Example 23.1. B Cell Activation

To determine optimal B cell culture and activation conditions compatible with efficient lipid transfection for gene editing, we compared surface expression of CD86 and low density lipoprotein receptor (LDLR) in B cells cultured under various conditions. CD86 is a costimulatory receptor upregulated on B cells upon their activation, while LDLR has been shown to be involved in ApoE-mediated LNP uptake.

Healthy human donor PBMCs were obtained commercially (Hemacare) and B cells were isolated by CD19 positive selection using CD19 MicroBeads (Milteni Biotec, Catalog, 130-050-301) following the manufacturer's protocol using LS columns (Milteni Biotec, 130-042-401) on a QuadroMACS separator (Milteni Biotec, Catalog, 130-091-051).

### Example 23.1.1. B Cell Culture Media Preparation

B cell culture media compositions used below are described in Tables 63 and 64. “IMDM Base Media” consists of IMDM Media, supplemented with 1% Penicillin/Streptomycin. “StemSpan SFEM Base Media” consists of StemSpan SFEM Media, supplemented with 1% Penicillin/Streptomycin. In addition to above mentioned components, media may contain serum, cytokines and activation factors. Media components are described in Table 63. and B cell culture media compositions are described in Table 64.

TABLE 63
Media components

| Media Components | | Concentration | | Vendor | Catalog# |
|---|---|---|---|---|---|
| Base | IMDM | | | Corning | 10-016-CV |
| | Penicillin/Streptomycin | 1% | | Corning | 30-002-CI |
| Base | Stem Span SFEM | | | StemCell | 9650 |
| | | | | Technologies | |
| | Penicillin/Streptomycin | 1% | | Corning | 30-002-CI |
| Serums | Fetal Bovine Serum (FBS) | 10% | | Gibco | A3 840201 |
| | Human AB Serum (HABS) | 5% | | Gemini Bio- | 100-512 |
| | | | | Products | |
| Cytokines | hIL-2 | 50 | ng/ml | Peprotech | 200-02 |
| | hIL-4 | 200 | U/ml | Stemcell | 78147.1 |
| | hIL-10 | 50 | ng/ml | Peprotech | 200-10 |
| | hIL-15 | 10 | ng/ml | Peprotech | 200-15 |
| | hIL-21 | 20 | ng/mL | Peprotech | 200-21 |
| Activation | BAFF | 10 | ng/ml | R&D | 2149-BF-010 |
| Factors | rhinsulin | 5 | μg/ml | Sigma | 91077C |
| | rhTransferrin | 50 | μg/mL | Sigma | T8158 |
| | | transferrin | | | |
| | MEGACD40L | 1 or 10 or 100 | | Enzo Life | ALX-522- |
| | | ng/ml | | Sciences | 110-C010 |
| | CpG ODN 2006 | 1 | μg/mL | Invivogen | TLR-2006 |

TABLE 64
B cell media compositions

| | | | Supplements | |
|---|---|---|---|---|
| B Cell | Base Media | | & Activation | |
| Media | (+pen/strep) | Cytokines | Factors | Serum |
| 1 | Stem Span | none | none | 10% FBS |
| 2 | Stem Span | IL-4 | BAFF | 10% FBS |
| 3 | Stem Span | IL-2, IL-10, IL-15 | CpG ODN | 10% FBS |
| 4 | Stem Span | IL-2, IL-10, IL-15, | insulin, | 10% FBS |
| | | IL-21 | transferrin | |
| 5 | IMDM | none | none | 10% FBS |
| 6 | IMDM | IL-4 | BAFF | 10% FBS |
| 7 | IMDM | IL-2, IL-10, IL-15 | CpG ODN | 10% FBS |
| 8 | IMDM | IL-2, IL-10, IL-15, | insulin, | 10% FBS |
| | | IL-21 | transferrin | |
| 9 | Stem Span | IL-2, IL-10, IL-15 | CpG ODN | 5% HABS |

Following MACS isolation, B cells were activated by culturing in duplicate at 100,000 cells/well in B cell media 1, 2, 3, 5, 6 or 7 as described in Table 64 supplemented with 1, 10, or 100 ng/ml MEGACD40L.

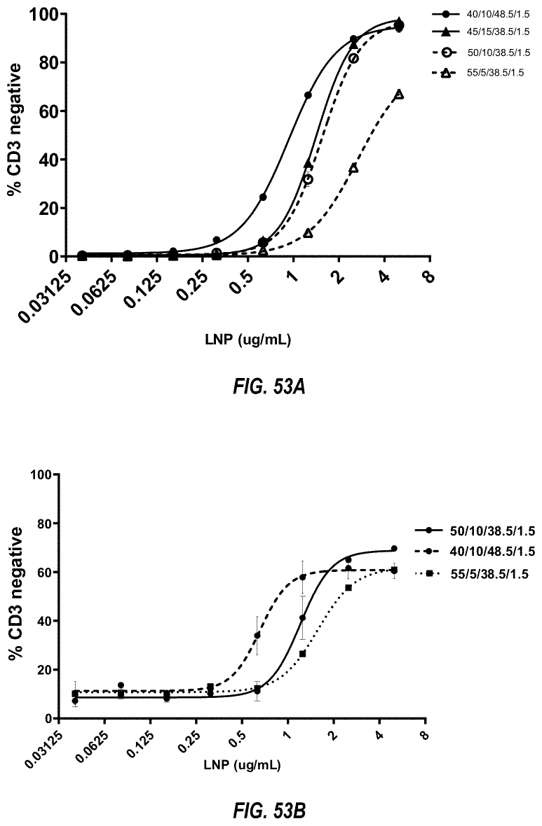

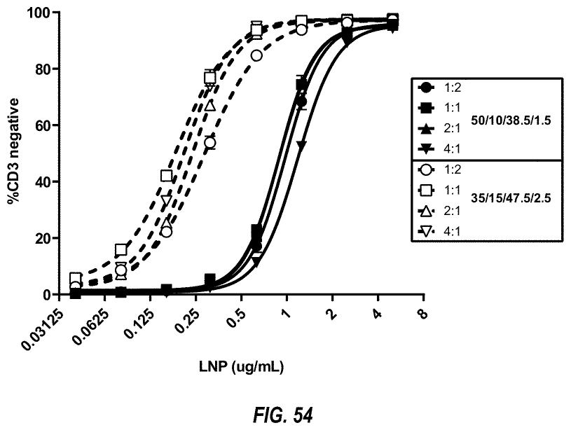

On day 5 post activation, B cells were phenotyped by flow cytometry to determine surface expression of CD86 and LDLR. Briefly, B cells were incubated with antibodies targeting CD20 (Biolegend, 302322), CD86 (Biolegend, 374216), and LDLR (BD, 565653). Cells were subsequently stained with a viability dye (DAPI, Biolegend, 422801), washed, processed on a Cytoflex instrument (Beckman Coulter) and analyzed using the FlowJo software package. B cells were gated on size and viability status, followed by CD20 expression, followed by CD86 and LDLR expression on the CD20+ cells. Table 65 and FIGS. 56A-D show the percentage of CD86+ cells and the percentage of LDLR+ cells among B cells.

MEGACD40L at 100 ng/m in base media led to upregulation of CD86 or LDLR without additional activators or cytokines. CD86+ and LDLR+ cells increased with addition of IL-4 and BAFF in a MEGACD40L dependent manner. Supplementation with CpG ODN, IL-2, IL-10 and IL-15 lead to high percentage of CD86+ and LDLR+ cells regardless of CD40L levels. These trends were consistent across IMDM and StemSpan media.

TaBLE 65
CD86 and LDLR expression in B cells

| | CD40L | % CD86+ | | % LDLR+ | |
|---|---|---|---|---|---|
| B Cell Media | (ng/ml) | Mean | SD | Mean | SD |
| #1 (StemSpan) | 1 | 13.05 | 4.17 | 8.46 | 2.07 |
| | 10 | 18.50 | 0.57 | 13.90 | 0.99 |
| | 100 | 59.15 | 6.58 | 55.10 | 5.09 |
| #2 (StemSpan + IL-4 + BAFF) | 1 | 64.65 | 4.60 | 33.90 | 3.11 |
| | 10 | 74.50 | 3.11 | 37.95 | 1.06 |
| | 100 | 84.15 | 7.57 | 57.00 | 5.09 |
| #3 (StemSpan + IL-2/10/15 + | 1 | 73.20 | 1.27 | 89.50 | 3.96 |
| CpG ODN) | 10 | 77.10 | 4.81 | 83.85 | 12.94 |
| | 100 | 82.70 | 2.83 | 78.95 | 3.46 |
| #5 (IMDM) | 1 | 8.71 | 1.16 | 5.60 | 0.52 |
| | 10 | 10.87 | 2.45 | 8.23 | 2.14 |
| | 100 | 33.55 | 1.63 | 30.95 | 4.45 |
| #6 (IMDM + IL-4 + BAFF) | 1 | 52.65 | 1.06 | 33.15 | 0.64 |
| | 10 | 60.35 | 4.74 | 38.80 | 2.69 |
| | 100 | 88.15 | 0.78 | 62.55 | 2.33 |
| #7 (IMDM + IL-2/10/15 + | 1 | 81.85 | 1.06 | 76.35 | 1.63 |
| CpG ODN) | 10 | 80.40 | 0.57 | 74.65 | 0.21 |
| | 100 | 77.45 | 1.20 | 71.70 | 2.26 |

### Example 23.2. B Cell Expansion

Following primary activation, B cells must undergo differentiation into plasmablasts and then into plasma cells to acquire the ability to secrete large amounts of protein. Once B cells differentiate into plasma cells, expansion halts. Therefore, during the engineering process, it is important to maximize B cell expansion during the phases of primary B cell activation and plasmablast differentiation (secondary activation). To determine media conditions for improved B cell expansion and differentiation to plasma cells, we cultured B cells in various media and measured fold expansion 7 and 14 days after initial culturing.

Briefly, B cells were isolated from PBMC as described in Example 23.1. Following MACS isolation, CD19+ B cells were cultured in duplicate at 1,000,000 cells/well in B cell media 3 or B cell media 7 as described in Table 64 supplemented with 1, 10, or 100 ng/ml MEGACD40L. To induce B cell differentiation into plasmablasts and measure expansion, B cells were cultured at 100,000 cells/well in B cell media 4 or B cell media 8 supplemented with 1, 10, or 100 ng/ml MEGACD40L. Cells were counted using a Vi-CELL cell counter (Beckman Coulter) on Day 7 and Day 14 post activation, and fold expansion was calculated by dividing cell yield by the starting cell count at the time of activation.

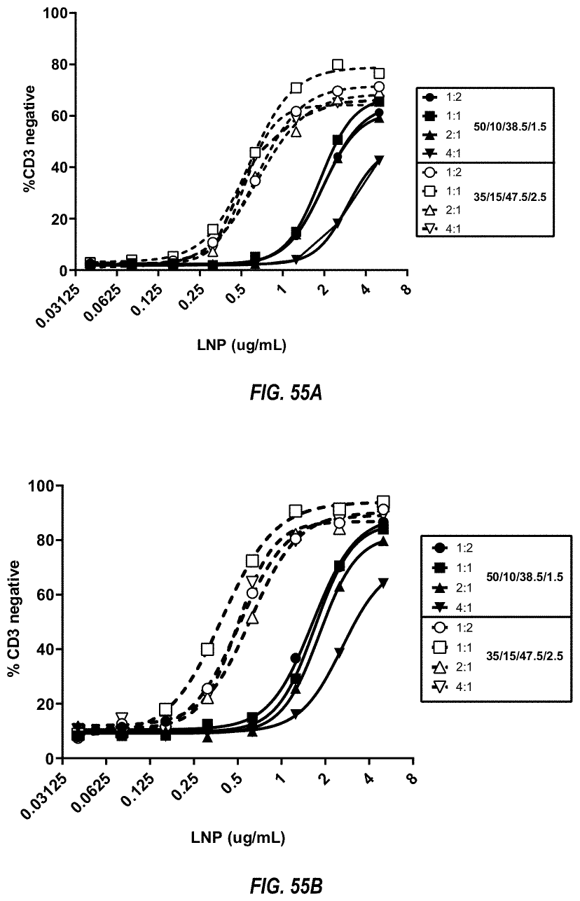

Expansion results are shown in Tables 66 and 67 and FIGS. 57A-B. For primary expansion, fold expansion was highest for B cells cultured in StemSpan in 100 ng/ml MEGACD40L as seen in Table 66 and FIG. 57A. Expansion is significantly lower with lower amounts of MEGACD40L regardless of media. Culture in IMDM resulted in lower expansion rates across all conditions tested compared to StemSpan. B cell culture and plasmablast differentiation in StemSpan resulted in higher fold expansion compared to IMDM, as did culture in higher MEGACD40L concentration. In an additional test, approximately 20-fold expansion was achieved using Stemspan base media and human serum in place of FBS.

TABLE 66
B cell fold expansion after primary activation

| | | Stemspan | | IMDM | |
|---|---|---|---|---|---|
| | CD40L | Replicate | Replicate | Replicate | Replicate |
| Expansion | (ng/ml) | 1 | 2 | 1 | 2 |
| Primary Expansion | 100 | 21.42 | 24.08 | 5.81 | 5.32 |
| Day 7 | 10 | 10.5 | 8.12 | 3.22 | 2.59 |
| | 1 | 3.5 | 5.04 | 2.87 | 1.96 |
| Primary Expansion | 100 | 33.81 | 41.58 | 14.21 | 17.5 |
| Day 14 | 10 | 23.38 | 17.99 | 6.51 | 6.02 |
| | 1 | 8.82 | 10.43 | 2.94 | 2.8 |

TABLE 67
B cell fold expansion after secondary activation

| | | Stemspan | | IMDM | |
|---|---|---|---|---|---|
| | CD40L | Replicate | Replicate | Replicate | Replicate |
| Expansion | (ng/ml) | 1 | 2 | 1 | 2 |
| Secondary | 100 | 18.08 | 15.04 | 7.36 | 8.32 |
| Expansion | 10 | 7.68 | 5.76 | 4.8 | 3.52 |
| Day 7 | 1 | 2.24 | 4.96 | 2.88 | 2.4 |
| Secondary | 100 | 42.68 | 32.34 | 10.78 | 15.4 |
| Expansion | 10 | 13.2 | 15.4 | 3.96 | 5.5 |
| Day 14 | 1 | 14.08 | 2.64 | 2.42 | 1.738 |

### Example 23.3. Lipid Screen in Activated B Cells

LNPs formulated with different ionizable or PEG lipids were tested for B cell editing efficacy.

A leukopak from a healthy human donor was obtained commercially (Hemacare) and B cells were isolated by CD19 positive selection using the StraightFrom Leukopak CD19 MicroBead kit (Miltenyi, 130-117-021) on a MultiMACS Cell24 Separator Plus instrument. Following MACS isolation, CD19+ B cells were activated in B Cell Media 3 or B Cell Media 7, each supplemented with 100 ng/ml MEGACD40L. Two days following activation, B cells were treated with LNPs delivering Cas9 mRNA and gRNA G013006 (SEQ ID NO: 708) targeting TRAC. LNPs were generally prepared as Example 1 using the ionizable and PEG lipids described in Table 68 with the lipid composition expressed as the molar ratio of ionizable lipid/cholesterol/DSPC/PEG, respectively. LNPs were pre-incubated with 1 μg/ml ApoE3 (Peprotech, 350-02) at 10 μg/ml total RNA cargo for 15 minutes in IMDM or StemSpan base media supplemented with 10% FBS (Gibco, A3840201). The pre-incubated LNPs were added at a 1:1 ratio v/v to B cells resulting in a final concentration of 100 ng/ml MEGACD40L in B cell Media 3 or 7 at an LNP dose of total RNA cargo of 5 μg/ml as indicated in Table 68.

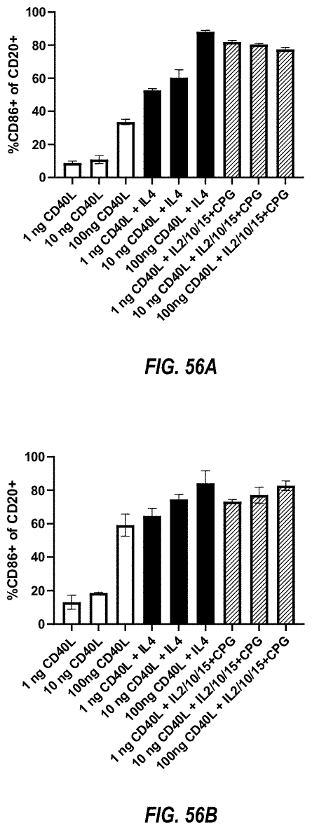

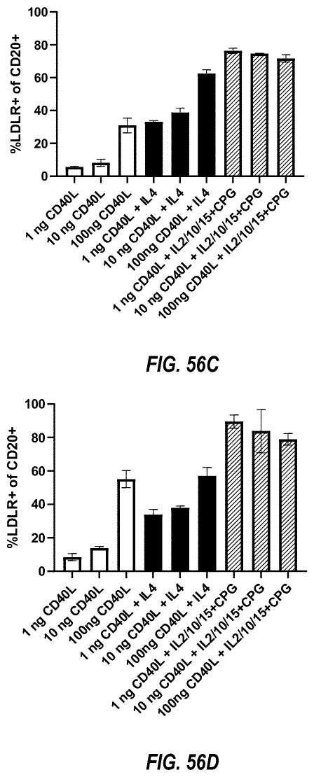

Five days post LNP treatment, cells were collected and NGS analysis was performed as described in Example 1. Table 68 and FIGS. 58A-B show percent editing following LNP treatment in various media. Efficient editing was evident using LNPs formulated with Lipid A, Lipid C or Lipid D. See Table 90 below for lipid structures. Editing was more efficient in B cells cultured in StemSpan compared to IMDM.

TABLE 68
Mean percent editing in B cells following editing with described
lipid compositions.

| | LNP | | | | |
|---|---|---|---|---|---|
| | Ionizable | | | 5 ug/ml | |
| Media | lipid | PEG lipid | Lipid composition | Mean | SD |
| IMDM | Lipid A | 2kDMG | 50/10/38.5/1.5 | 5.50 | 0.71 |
| | Lipid C | 2kDMG | 50/10/38.5/1.5 | 13.00 | 0.00 |
| | Lipid D | 2kDMG | 50/10/38.5/1.5 | 32.00 | 0.00 |
| | Lipid E | 2kDMG | 50/10/38.5/1.5 | 1.00 | 0.00 |
| | Lipid F | 2kDMG | 50/10/38.5/1.5 | 0.50 | 0.71 |
| | Lipid G | 2kDMG | 50/10/38.5/1.5 | 1.00 | 0.00 |
| | Lipid A | 2kDMG | 50/9/38/3 | 5.50 | 0.71 |
| | Lipid E | 2kDMG | 50/9/38/3 | 0.00 | 0.00 |
| | Lipid G | 2kDMG | 50/9/38/3 | 1.00 | 0.00 |
| | Lipid A | Lipid H | 50/10/38.5/1.5 | 16.50 | 0.71 |
| | Lipid A | Lipid J | 50/10/38.5/1.5 | 7.00 | 0.00 |
| | Untreated | | | 0.00 | 0.00 |
| SFEM | Lipid A | 2kDMG | 50/10/38.5/1.5 | 17.50 | 3.54 |
| | Lipid C | 2kDMG | 50/10/38.5/1.5 | 43.50 | 2.12 |
| | Lipid D | 2kDMG | 50/10/38.5/1.5 | 59.00 | 1.41 |
| | Lipid E | 2kDMG | 50/10/38.5/1.5 | 6.50 | 0.71 |
| | Lipid F | 2kDMG | 50/10/38.5/1.5 | 1.00 | 0.00 |
| | Lipid G | 2kDMG | 50/10/38.5/1.5 | 1.00 | 0.00 |
| | Lipid A | 2kDMG | 50/9/38/3 | 13.00 | 1.41 |
| | Lipid E | 2kDMG | 50/9/38/3 | 1.00 | 0.00 |
| | Lipid G | 2kDMG | 50/9/38/3 | 0.00 | 0.00 |
| | Lipid A | Lipid H | 50/10/38.5/1.5 | 30.00 | 1.41 |
| | Lipid A | Lipid J | 50/10/38.5/1.5 | 10.00 | 0.00 |

| | | |
|---|---|---|
| Untreated | 0.00 | 0.00 |

### Example 23.4. ApoE Conditions for B Cell Editing with LNPs

To determine editing efficacy using varying doses of LNP preincubated with either ApoE3 or ApoE4, surface expression of B2M protein was assessed following editing in B cells with guides targeting B2M.

B cells (Hemacare) were thawed and activated in Stemspan SFEM media with 1 ug/ml CpG ODN 2006 (Invivogen, cat. tlrl-2006-1), 50 ng/ml IL-2 (Peprotech, cat. 200-02), 50 ng/ml IL-10 (Peprotech, cat. 200-10), 10 ng/ml IL-15 (Peprotech, cat. 200-15), 1 ng/ml MegaCD40L (Enzo Life Sciences, cat. ALX-522-110-0000), 1% penicillin-streptomycin and 5% human AB serum. B cells were considered activated in the presence of 1 ng/mL MegaCD40L (Enzo Life Sciences, cat. ALX-522-110-0000) and 1 ug/mL CpG ODN 2006 (Invivogen, cat. tlrl-2006-1). Two days following activation, B cells were treated with LNPs delivering Cas9 mRNA and gRNA G000529 (SEQ ID NO: 701) targeting B2M. LNPs were generally prepared as Example 1 using the ionizable lipids described in Table 69 with the lipid composition of 50/10/38.5/1.5, expressed as the molar ratio of ionizable lipid/cholesterol/DSPC/PEG, respectively. LNPs were preincubated at 37° C. for about 5 minutes with either ApoE3 (Peprotech 350-02) or ApoE4 (Peprotech 350-04) at 1.25 ng/ml in Table 69. The pre-incubated LNPs were added to B cells at amounts of total RNA cargo as indicated in Table 69. Five days post LNP treatment, cells were phenotyped by flow cytometry. Briefly, B cells were incubated with antibodies targeting B2M (Biolegend, cat. 395806), CD19 (Biolegend, cat. 302205), CD20 (Biolegend, cat. 302322), CD86 (Biolegend cat. 305420). Cells were subsequently washed in DAPI (Thermo Fisher, cat. D1306) diluted 1:3703 in PBS, processed on a Cytoflex instrument (Beckman Coulter) and analyzed using the FlowJo software package. B cells were gated on size, singlets, and live cells.

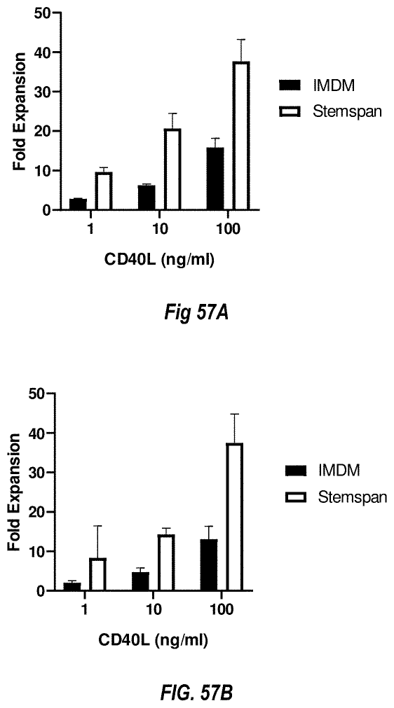

Table 69 and FIG. 59 show percent B2M negative B cells following editing with LNPs formulated with Lipid A or Lipid D that had been preincubated with ApoE3 or ApoE4. B cells edited with LNPs with formulated with either Lipid A or Lipid D ionizable lipids showed increased percentage of B2M negative cells.

TABLE 69
Mean percent B2M negative B cells following editing with
different LNP formulation preincubated with ApoE3 or ApoE4.

| LNP | Total RNA | Lipid A | | Lipid D | |
|---|---|---|---|---|---|
| Preincubation | ug/ml | Mean | SD | Mean | SD |
| ApoE3 | 0 | 1.84 | 0.54 | 1.84 | 0.14 |
| | 2.5 | 53.50 | 1.56 | 37.00 | 0.42 |
| | 5 | 53.05 | 0.92 | 30.30 | 1.56 |
| | 10 | 50.00 | 0.57 | 20.50 | 1.13 |
| ApoE4 | 0 | 1.27 | 0.30 | 1.91 | 0.44 |
| | 2.5 | 60.30 | 0.14 | 40.80 | 1.98 |
| | 5 | 51.45 | 3.32 | 27.75 | 2.62 |
| | 10 | 47.55 | 0.92 | 19.50 | 0.28 |

### Example 24. Editing Time Course in B Cell Using Lipid Nanoparticles

To determine an efficacious interval between B cell activation and editing with LNP, surface expression of B2M protein was assessed following editing in B cells with guides targeting B2M.

B cells (Hemacare) were thawed and activated in Stemspan SFEM media with 1 ug/ml CpG ODN 2006 (Invivogen, cat. tlrl-2006-1), 50 ng/ml IL-2 (Peprotech, cat. 200-02), 50 ng/ml IL-10 (Peprotech, cat. 200-10), 10 ng/ml IL-15 (Peprotech, cat. 200-15), 1 ng/ml MegaCD40L (Enzo Life Sciences, cat. ALX-522-110-0000), 1% penicillin-streptomycin and 5% human AB serum. B cells were considered activated in the presence of 1 ng/mL MegaCD40L (Enzo Life Sciences, cat. ALX-522-110-0000) and 1 ug/mL CpG ODN 2006 (Invivogen, cat. tlrl-2006-1). B cells were treated with LNPs delivering mRNA encoding Cas9 and gRNA G000529 (SEQ ID NO: 701) targeting B2M at intervals across two independent experiments. The first (shown in Table 70) included an edit on thaw (day −1), activating the cells the following day, and an edit on day 0 and at each subsequent day until day 5 post activation. The second (shown in Table 71) included thawing and activating cells on the same day (day 0) and performing the edit on day 6 through day 10.

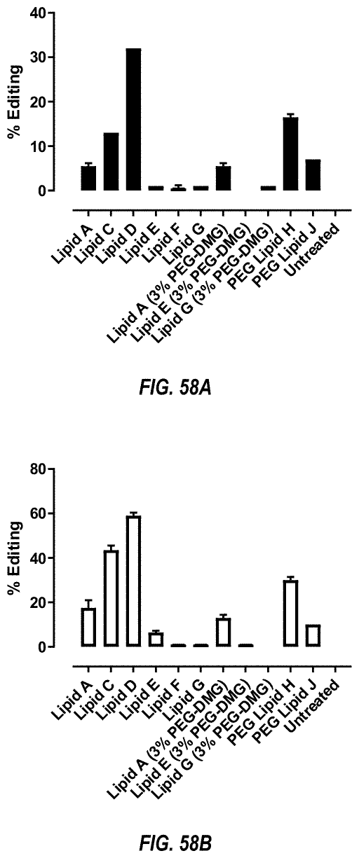

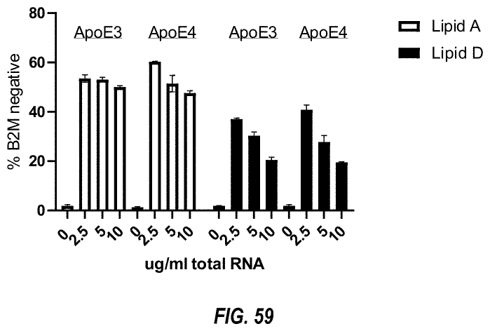

Flow cytometry analysis was performed separately—6 days after each edit. LNPs were generally prepared as Example 1 using the ionizable lipids described in Tables 70-71 with the lipid composition of 50/10/38.5/1.5, expressed as the molar ratio of ionizable lipid/cholesterol/DSPC/PEG, respectively. LNPs were preincubated at 37° C. for about 5 minutes with ApoE4 (Peprotech 350-04) at 1.25 ng/ml. The pre-incubated LNPs were added to B cells at final concentration 2.5 ug/ml total RNA cargo, and human serum final concentration of 2.5%. Six days post LNP treatment, cells were phenotyped by flow cytometry. Briefly, B cells were incubated with antibody targeting B2M (Biolegend, cat. 395806) for 20 minutes at 4c. Cells were subsequently washed in DAPI (Thermo Fisher, cat. D1306) diluted (3.8 uM) in PBS, processed on a Cytoflex instrument (Beckman Coulter) and analyzed using the FlowJo software package. B cells were gated on singlets, and live cells, and compared to the no LNP negative control for loss of B2M. Tables 70-71 and FIGS. 60A-B show the percent of B2M negative cells following LNP treatment. Effective editing was observed in B cells from the same day of activation through 10 days post editing, with a peak between days 3 and 6.

TABLE 70
Mean B2M negative cells following editing at intervals from one
day before to 5 days after activation

| | Lipid A | | Lipid D | |
|---|---|---|---|---|
| Timepoint | Mean | SD | Mean | SD |
| Day-1 | 5.19 | 0.77 | 1.30 | 0.17 |
| Day 0 | 55.93 | 4.20 | 27.87 | 3.69 |
| Day 1 | 80.27 | 0.99 | 38.17 | 0.99 |
| Day 2 | 81.20 | 2.80 | 57.53 | 1.66 |
| Day 3 | 84.47 | 2.86 | 61.23 | 2.63 |
| Day 4 | 85.00 | 0.20 | 63.20 | 1.91 |
| Day 5 | 87.03 | 4.69 | 70.53 | 3.34 |

TABLE 71
Mean B2M negative cells following editing
at intervals from 6 to 10 days after activation

| Timepoint | Mean | SD |
|---|---|---|
| Day 6 | 86.10 | 2.55 |
| Day 7 | 79.77 | 1.70 |
| Day 8 | 75.07 | 6.82 |
| Day 9 | 62.03 | 7.17 |
| Day 10 | 52.47 | 5.15 |

### Example 25. Editing in B Cells Using DNA Protein Kinase Inhibitors

The effect of DNA protein kinase inhibitors (DNAPKi) on editing efficiency in B cells was assessed.

B cells were isolated as in Example 23.3 and frozen until needed. B cells were thawed and cultured in in B cell media 9 as described in Table 64 supplemented with 1 ng/ml MEGACD40L. Following two days of culture, cells were harvested and resuspended at 100,000 cells/100 μl in StemSpan base media without human serum, supplemented with 2× the final concentration of the cytokine and activation factor cocktail used in B cell media 9 prior to treatment with LNPs delivering mRNA encoding Cas9 (SEQ ID NO: 6) and gRNA G000529 (SEQ ID NO: 701) targeting B2M.

LNPs were generally prepared as Example 1 with the lipid composition of 50/10/38.5/1.5, expressed as the molar ratio of ionizable lipid/cholesterol/DSPC/PEG, respectively. LNPs were preincubated at a concentration of 5 μg/ml total RNA cargo with 1.25 μg/ml ApoE4 (Peprotech, 350-04) at 37° C. for about 15 minutes in StemSpan base media supplemented with 5% human AB serum (Gemini Bio-Products, 100-512). The pre-incubated LNPs were added to B cells at a final concentration 2.5 ug/ml total RNA cargo followed by addition of 0.25 ug/ml DNAPK inhibitor Compound 1, Compound 3, or Compound 4.

B cells were phenotyped for the presence of B2M surface protein on day 7 post LNP treatment. For this, B cells were incubated with antibodies targeting CD86 (Biolegend, 374216) and B2M (Biolegend, 316312). Cells were subsequently stained with a viability dye (Biolegend, 422801), washed, processed on a Cytoflex instrument (Beckman Coulter) and analyzed using the FlowJo software package. B cells were gated on size and viability status, followed by B2M expression on the total live population. Percent B2M negative cells is shown in Table 72. Increased percentage of B2M negative B cells were observed in the presence of DNAPKi compared to no DNAPKi, indicating increased gene editing.

TABLE 72
Percentage of B2M negative cells following editing with
DNAPKi and LNP targeting B2M.

| | % B2M− | |
|---|---|---|
| Sample | Mean | SD |
| No Inhibitor | 11.2 | 1.5 |
| Compound 1 | 19.2 | 2.3 |
| Compound 3 | 27.4 | 2.6 |
| Compound 4 | 24.1 | 1.0 |
| No edit | 2.5 | 0.5 |

### Example 25.1. Editing in B Cells from Multiple Donors Using DNAPK Inhibitors

B cells were isolated from PBMC derived from 3 donors as described in Example 23.1. Following MACS isolation, CD19+ B cells were activated in Stemspan base media with 1 ug/ml CpG ODN 2006 (Invivogen, TLR-2006), 2.5% human AB serum (Gemini Bio-Products, 100-512), 1% penicillin-streptomycin (ThermoFisher, 15140122), 50 ng/ml IL-2 (Peprotech, 200-02), 50 ng/ml IL-10 (Peprotech, 200-10), and 10 ng/ml IL-15 (Peprotech, 200-15) and 1 ng/ml CD40L (Enzo Life Sciences, ALX-522-110-0010). Two days following activation, B cells were treated with LNPs delivering mRNA encoding Cas9 (SEQ ID NO: 6) and gRNA G000529 (SEQ ID NO: 701) targeting B2M. B cells were plated at 50,000 cells per well in triplicate as indicated in Table 73 in complete Stemspan media as described above.

LNPs were generally prepared as in Example 1 with the lipid composition of 50/10/38.5/1.5, expressed as the molar ratio of ionizable lipid/cholesterol/DSPC/PEG, respectively. LNPs were preincubated at 37° C. for 15 minutes with Stemspan media containing 1 ug/ml CpG ODN 2006, 2.5% human AB serum, 1% penicillin-streptomycin, 50 ng/ml IL-2, 50 ng/ml IL-10, and 10 ng/ml IL-15, 1 ng/ml CD40L, and 1.25 ug/mL ApoE4. The pre-incubated LNPs were added to B cells at a final concentration 2.5 ug/ml total RNA cargo followed by addition of 0.25 ug/ml DNAPK inhibitor Compound 1 or Compound 4. Seventy-two hours post-LNP addition, cells were washed, resuspended in Stemspan media containing 1 ug/ml CpG ODN 2006, 2.5% human AB serum, 1% penicillin-streptomycin, 50 ng/ml IL-2, 50 ng/ml IL-10, and 10 ng/ml IL-15, and 100 ng/ml CD40L and transferred to a 48-well plate.

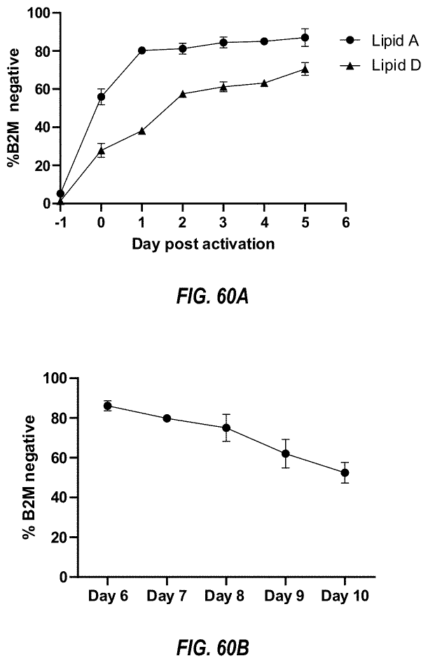

Seven days post-LNP treatment, cells were phenotyped by flow cytometry. Briefly, B cells were incubated with antibodies targeting CD19 (Biolegend, 363010A), CD20 (Biolegend, 302322), CD86 (Biolegend, 374216) and B2M (Biolegend, 395806) followed by viability dye DAPI (Biolegend, 422801). Cells were subsequently washed and processed on a Cytoflex instrument (Beckman Coulter) and analyzed using the FlowJo software package. B cells were gated on size and viability status, followed by B2M expression on the total live population. Table 73 and FIG. 61 show mean percent of B2M negative cells following editing with DNAPK inhibitors. Addition of DNAPK inhibitors moderately improved editing efficiency.

TABLE 73
Mean percent B2M negative cells following
editing with DNAPK inhibitors

| | Unedited, | | | | | | | | | | | |
|---|---|---|---|---|---|---|---|---|---|---|---|---|
| | No inhibitor | | | No inhibitor | | | Compound 1 | | | Compound 4 | | |
| Donor | Mean | SD | N | Mean | SD | N | Mean | SD | N | Mean | SD | N |
| Donor | 3.74 | 1.62 | 3 | 50.57 | 1.54 | 3 | 56.41 | 4.39 | 3 | 59.42 | 4.16 | 3 |
| 150 | | | | | | | | | | | | |
| Donor | 5.11 | 0.06 | 2 | 27.60 | 4.16 | 3 | 36.77 | 1.79 | 3 | 34.88 | 8.44 | 3 |
| 200 | | | | | | | | | | | | |
| Donor | 0.70 | 0.43 | 3 | 45.61 | 3.23 | 3 | 56.28 | 3.01 | 3 | 57.59 | 3.52 | 3 |
| 340 | | | | | | | | | | | | |

### Example 26. Insertion into B Cells Using LNP Delivery

B cells were assessed for insertion efficacy using a combination of LNPs and AAV nucleic acid delivery.

B cells were isolated as Example 23.3. and were activated in B cell media 9 as described in Table 64, supplemented with 1 ng/ml MEGACD40L. Two days following activation, B cells were treated with LNPs delivering mRNA encoding Cas9 (SEQ ID NO. 6) and gRNA G000529 (SEQ ID NO: 701) targeting the B2M locus as well as with AAV6 for GFP template insertion into the B2M locus (SEQ ID NO. 722) driven by the EF1α promoter. LNPs were generally prepared as described in Example 1 using Lipid A and Lipid D as the ionizable lipids with the lipid composition of 50/10/38.5/1.5, expressed as the molar ratio of ionizable lipid/cholesterol/DSPC/PEG, respectively. LNP was added to StemSpan base media supplemented with 5% human AB serum (Gemini Bio-Products, 100-512) together with 1 μg/ml APOE3 (Peprotech, 350-02) One hundred thousand cells/well were cultured in StemSpan base media with no human serum and 4× the concentration of the cytokine/activation factor cocktail detailed above. The LNP mixture was incubated at 37° C. for 15 mins before mixing 1:1 v/v with B cells. Immediately after combining cells and LNP, AAV6 was added at an MOI of 1.5×10\hat{ }5 genome copies, 1:1 v/v with the B cell-LNP mixture, resulting in a final concentration of 5 μg/ml LNP.

B cells were phenotyped for GFP expression on Day 7. For B cell phenotyping by flow cytometry, B cells were stained with antibodies targeting CD19 (Biolegend, 302218), CD20 (Biolegend, 302322), CD86 (Biolegend, 374216) and B2M (Biolegend, 316312). Cells were subsequently stained with a viability dye (Biolegend, 422801), washed, and processed on a Cytoflex instrument (Beckman Coulter). Results were analyzed using the FlowJo software package. B cells were gated based on size and viability, followed by GFP expression on the total live population. As shown in Table 74, the percentage of B cells expressing GFP was 29.5% on Day 7 post-treatment with Lipid A, and 14.5% with Lipid D. Minimal GFP expression was observed in negative control conditions that received no treatment, LNP only, or AAV only.

TABLE 74
Percent B2M negative and percent GFP positive following LNP
and AAV treatment

| | % B2M negative | | % GFP positive | |
|---|---|---|---|---|
| Sample | Mean | SD | Mean | SD |
| Untreated | 1.83 | 0.25 | 0.00 | 0.00 |
| AAV Only | 1.46 | 0.50 | 2.28 | 0.66 |
| Lipid A Only | 62.35 | 0.35 | 0.00 | 0.00 |
| Lipid A + AAV | 42.45 | 0.92 | 29.50 | 5.52 |
| Lipid D Only | 58.45 | 0.21 | 0.00 | 0.00 |
| Lipid D + AAV | 27.05 | 2.76 | 14.25 | 0.07 |

### Example 27. Editing in NK Cell Using Lipid Nanoparticles

LNPs formulated with different ionizable or PEG lipids were tested for NK cell editing efficacy.

NK cells were isolated from a commercially obtained leukopak using the EasySep Human NK Cell Isolation Kit (STEMCELL, Cat. No. 17955) according to the manufacturers protocol. Following isolation, NK cells were cultured at 1:1 ratio with K562-41BBL feeder cells in RPMI 1640 media with 10% FBS, 500 U/mL IL-2, 5 ng/ml IL-15, and 10 ng/ml IL-21 for 7 days.

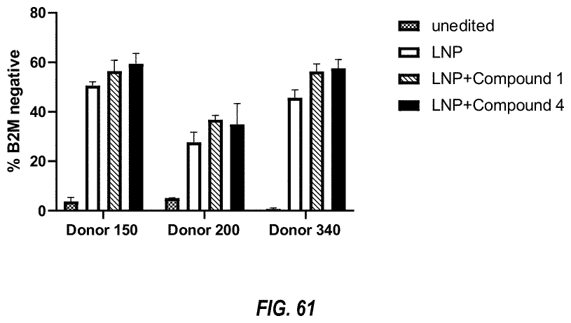

Seven days following activation, NK cells were treated with LNPs delivering Cas9 HiBiT mRNA and gRNA G013006 (SEQ ID NO: 708) targeting TRAC. LNPs were generally prepared as Example 1 using the ionizable and PEG lipids described in Table 75 with the lipid composition expressed as the molar ratio of ionizable lipid/cholesterol/DSPC/PEG, respectively. LNPs were preincubated at 37° C. for about 15 minutes in RPMI 1640 with 10% FBS. The pre-incubated LNPs were added to NK cells at 2.5 ug of total RNA cargo in triplicate. At seven days post LNP treatment, cells were collected and NGS analysis was performed as described in Example 1. Table 75 and FIG. 62 show percent indels for NK cells treated with indicated LNP formulations. Editing was achieved with multiple lipid compositions. See Table 90 below for lipid structures.

TABLE 75
Mean percent editing in NK cells following editing with various
lipid compositions.

| LNP | | | % Editing | |
|---|---|---|---|---|
| Ionizable lipid | PEG lipid | Lipid composition | Mean | SD |
| Lipid A | 2kDMG | 50/10/38.5/1.5 | 57.93 | 2.02 |
| Lipid C | 2kDMG | 50/10/38.5/1.5 | 34.33 | 3.41 |
| Lipid D | 2kDMG | 50/10/38.5/1.5 | 38.90 | 1.39 |
| Lipid E | 2kDMG | 50/10/38.5/1.5 | 18.50 | 6.45 |
| Lipid F | 2kDMG | 50/10/38.5/1.5 | 0.20 | 0.10 |
| Lipid G | 2kDMG | 50/10/38.5/1.5 | 0.13 | 0.06 |
| Lipid A | 2kDMG | 50/9/38/3 | 19.83 | 2.83 |
| Lipid E | 2kDMG | 50/9/38/3 | 16.10 | 2.77 |
| Lipid G | 2kDMG | 50/9/38/3 | 4.20 | 0.20 |
| Lipid A | LIPID H | 50/10/38.5/1.5 | 17.23 | 4.31 |
| Lipid A | LIPID J | 50/10/38.5/1.5 | 41.80 | 5.99 |

| | | |
|---|---|---|
| Untreated | 0.00 | 0.00 |

### Example 28. Editing with Insertion Time Course in NK Cell Using Lipid Nanoparticles

To assess genomic insertion in NK cells, cells were treated with LNPs delivering mRNA encoding Cas9 (SEQ ID NO. 6) and gRNA G000562 (SEQ ID NO: 710) targeting AAVS1 followed by AAV encoding a GFP coding sequence flanked by regions of homology to the AAVS1 edit site (SEQ ID NO. 720 or SEQ ID NO. 721).

NK cells were isolated as in Example 27. For Donor 2, human primary NK cells were activated, expanded using K562-41BBL cells as feeder cells at the ratio of 1:1 in RPMI 1640 media with 10% FBS, 500 U/mL IL-2, and 5 ng/ml IL-15 for 7 days, cryopreserved, then thawed at the time of the experiment. For Donor 3, NK cells were isolated, activated and expanded using K562-41BBL cells at the ratio of 1:1 in RPMI 1640 media with 10% FBS, 500 U/mL IL-2, and 5 ng/ml IL-15 for 7 days and then used directly for editing. For Donor 4, NK cells were isolated, activated and expanded using K562-41BBL cells at the ratio of 1:1 in OpTmizer media with 5% human AB serum, 500 U/mL IL-2, and 5 ng/ml IL-15 for 7 days and then used directly for editing. NK cells were plated at 100,000 cells per well in triplicate in OpTmizer media with 2.5% human AB serum, 1% penicillin and streptomycin, 500 U/mL IL-2 and 5 ng/ml IL-15. For editing in RPMI 1640 medium, LNPs were preincubated with 10 ug/ml APOE3 at 37° C. for about 15 minutes in RPMI 1640 with 10% FBS, 500 U/mL IL-2 and 5 ng/ml IL-15. For editing in OpTmizer media, LNPs were preincubated with 10 ug/ml APOE3 at 37° C. for about 15 minutes in OpTmizer media with 2.5% human AB serum, 500 U/mL IL-2 and 5 ng/ml IL-15. The pre-incubated LNPs were added to NK cells suspended in the same media at a final concentration of 2.5 ug/ml, 5 ug/ml, or 10 ug/ml of total RNA cargo in triplicate. For a subset of samples, AAV at a multiplicity of infection (MOI) of 300,000 or 600,000 genome copies was added following editing. Cells were incubated for up to 14 days, replacing with fresh media at day 6 post editing.

### Example 28.1. Editing Efficiency in NK Cells

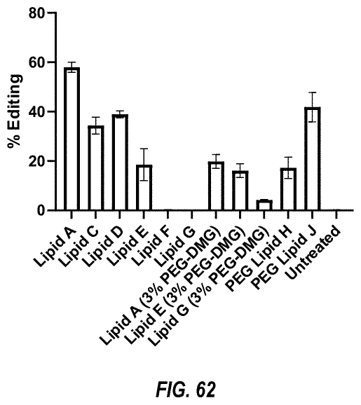

Each day post LNP treatment, cells were collected and NGS analysis was performed as described in Example 1. Editing was seen to substantially plateau by Day 8 in fresh cells (Donors 3 and 4) and Day 11 in frozen cells (Donor 2) at all three LNP doses. Endpoint editing at Day 14 post LNP treatment is shown in Table 76 and FIG. 63.

TABLE 76
Mean editing percentage in NK cells treated with varying doses
of LNP at 14 days post LNP treatment

| | | 2.5 ug/ml | | 5 ug/ml | | 10 ug/ml | | Unedited | |
|---|---|---|---|---|---|---|---|---|---|
| Donor | Condition | Mean | SD | Mean | SD | Mean | SD | Mean | SD |
| 2 | Expanded, | 84.23 | 0.91 | 85.83 | 0.49 | 85.63 | 0.32 | 0.27 | 0.12 |
| | frozen | | | | | | | | |
| 3 | Fresh, | 94.03 | 0.31 | 96.60 | 0.36 | 96.20 | 0.17 | 0.20 | 0.00 |
| | expanded | | | | | | | | |
| 4 | Fresh, | 94.87 | 0.35 | 95.90 | 0.17 | 95.43 | 0.21 | 0.10 | 0.00 |
| | expanded | | | | | | | | |

### Example 28.2. Insertion Efficiency in NK Cells

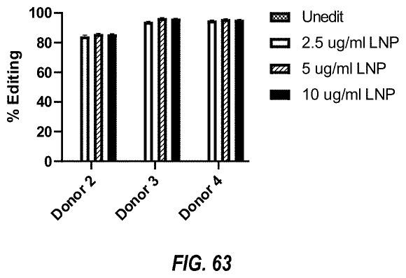

Seven days post LNP treatment, cells were assayed by flow cytometry to measure GFP insertion rates. Briefly, NK cells were incubated with antibodies targeting CD3 (Biolegend, Cat. No. 317336) and CD56 (Biolegend, Cat. No. 318310). Cells were subsequently washed, processed on a Cytoflex instrument (Beckman Coulter) and analyzed using the FlowJo software package. NK cells were gated on size, CD3/CD56 status, and GFP expression. High GFP-expressing cells were gated as targeted GFP insertion in AAVS1 locus and low GFP-expressing cells were gated as episomal retention. Table 77 and FIG. 64 show percent of NK cells with high GFP expression, indicating targeted insertion. In further assays, sequential gene disruption and sequence insertion edits were achieved in NK cells using LNPs.

TABLE 77
Percent of NK cells with high GFP expression seven days
following editing with LNP and AAV.

| Donor | Condition | Mean | SD | N |
|---|---|---|---|---|
| 2 | No AAV | 0.00 | 0.00 | 3 |
| | AAV only, no LNP | 1.47 | 0.41 | 3 |
| | 300K MOI | 40.03 | 2.05 | 3 |
| | 600K MOI | 40.63 | 3.91 | 3 |
| 3 | No AAV | 0.00 | 0.00 | 2 |
| | AAV only, no LNP | 1.24 | 0.48 | 2 |
| | 300K MOI | 53.77 | 0.85 | 3 |
| | 600K MOI | 58.40 | 0.92 | 3 |
| 4 | No AAV | 0.00 | 0.00 | 3 |
| | AAV only, no LNP | 1.63 | 0.18 | 3 |
| | 300K MOI | 63.20 | 0.87 | 3 |
| | 600K MOI | 67.13 | 1.55 | 3 |

### Example 29. Insertion into NK Cells Using DNAPK Inhibitors

NK cells were assessed for the impact of DNA protein kinase inhibitors (DNAPKi) on indel and insertion rates. NK cells were treated with LNPs delivering mRNA encoding Cas9 (SEQ ID NO. 6) and gRNA G000562 (SEQ ID NO: 710) targeting AAVS1 in the presence of DNA protein kinase inhibitors. A subset of samples was also treated with AAV encoding a GFP coding sequence flanked by regions of homology to the AAVS1 edit site (SEQ ID No. 721).

NK cells were isolated as in Example 27. Human primary NK cells were activated and expanded using K562-41BBL cells as feeder cells in OpTmizer media with 5% human AB serum, 500 U/mL IL-2, and 5 ng/ml IL-15 for 3 days. NK cells were plated at 50,000 cells per well in triplicate in OpTmizer media supplemented as described above with DNAPKi at concentrations indicated in Tables 78 and 79. LNPs were preincubated with 10 ug/ml APOE3 at 37° C. for about 15 minutes in OpTmizer media with 2.5% human AB serum, 500 U/mL IL-2 and 5 ng/ml IL-15. The pre-incubated LNPs were added to NK cells suspended in the same media at a final concentration of 10 ug/ml of total RNA cargo in triplicate. For a subset of samples, AAV encoding GFP flanked by regions homologous to the AAVS1 edit site were added at a multiplicity of infection (MOI) of 600,000 genome copies following editing. At seven days post LNP treatment, cells were phenotyped by flow cytometry as described in Example 28 and collected for NGS analysis as described in Example 1.

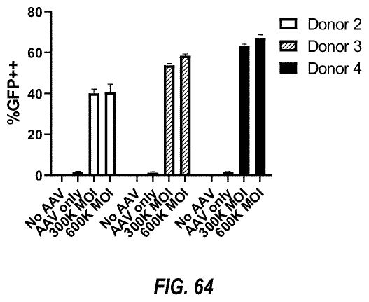

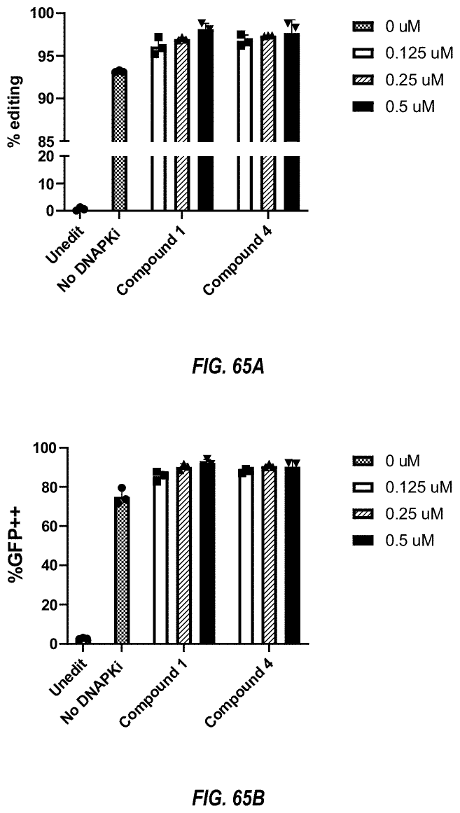

Tables 78 and 79 and FIGS. 65A and 65B show percent editing following treatment with LNP, AAV, and varying concentrations of the DNAPK inhibitors Compound 1 and Compound 4. Both indel formation and insertion increased in the presence of DNAPK inhibitors.

TABLE 78
Mean percent editing at AAVS1
with varying doses of DNAPKi

| | 0 uM | | 0.125 uM | | 0.25 uM | | 0.5 uM | |
|---|---|---|---|---|---|---|---|---|
| Sample | Mean | SD | Mean | SD | Mean | SD | Mean | SD |
| Unedited | 0.67 | 0.60 | | | | | | |
| No DNAPKi | 93.17 | 0.12 | | | | | | |
| Compound 1 | | | 96.10 | 1.01 | 96.97 | 0.21 | 98.13 | 0.65 |
| Compound 4 | | | 96.77 | 0.67 | 97.37 | 0.06 | 97.67 | 1.55 |

TABLE 79
Percent of NK cells with high GFP expression seven days
following editing with LNP, AAV and DNAPKi.

| | 0 uM | | 0.125 uM | | 0.25 uM | | 0.5 uM | |
|---|---|---|---|---|---|---|---|---|
| Sample | Mean | SD | Mean | SD | Mean | SD | Mean | SD |
| AAV only | 2.68 | 0.37 | | | | | | |
| No DNAPKi | 74.93 | 4.09 | | | | | | |
| Compound 1 | | | 85.47 | 2.48 | 90.23 | 1.66 | 92.30 | 1.66 |
| Compound 4 | | | 88.10 | 1.00 | 90.63 | 1.01 | 90.27 | 3.09 |

### Example 30. Cas9 Expression in Macrophage Cells after Lipid Nanoparticle Delivery

LNPs formulated with different ionizable or PEG lipids were tested for macrophage cell delivery efficacy.

Healthy human donor PBMCs were obtained commercially (Hemacare) and monocytes were isolated by CD14 positive selection using the CD14 MicroBeads, human (Miltenyi Biotec, Cat. 130-050-201) following the manufacturer's protocol. Following isolation, CD14+ monocyte cells were cultured and differentiated to macrophage cells in triplicate at 100,000 cells/well in RPMI-1640 media with 10 ng/mL GM-CSF (Stemcell, 78140.1) in tissue culture plates (Falcon, 353072).

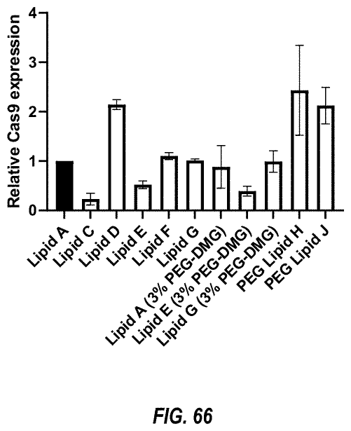

Five days following differentiation, cells were treated with LNPs delivering mRNA encoding Hibit-tagged Cas9 (SEQ ID NO. 7) and gRNA G013006 (SEQ ID NO: 708) targeting TRAC. LNPs were generally prepared as Example 1 using the ionizable and PEG lipids described in Table 80 with the lipid composition expressed as the molar ratio of ionizable lipid/cholesterol/DSPC/PEG, respectively. LNPs were preincubated at 37° C. at 5 ug/mL concentration for about 15 minutes in RPMI media containing 10% FBS and 10 ug/mL ApoE3. Media in cell plate was removed carefully and replaced with fresh RPMI media supplemented with 10% FBS, 1× Glutamax, 1× HEPES, 1% Penicillin/Streptomycin and 10 ng/mL GM-CSF. The pre-incubated LNPs were added to macrophage cells in 1:1 v/v ratio yielding a final LNP dose of 2.5 ug/mL total RNA cargo in triplicate. Cells were harvested 24 hours post LNP treatment and Cas9 protein levels were determined using the Nano-Glo® HiBiT Lytic Detection System (Promega, Cat. N3030) following manufacturer's direction. Luminescence was measured using the Biotek Neo2 plate reader. Linear regression was plotted on GraphPad using the protein number and luminescence readouts from the standard controls, forcing the line to go through X=0, Y=0. Used the Y=ax+0 equation to calculate number of proteins per cell lysate. Table 80 and FIG. 66 show Cas9 protein expression in macrophage cells transfected with various lipid compositions relative to the Lipid A with 1.5% PEG 2kDMG composition. Editing was achieved with multiple lipid compositions in macrophage cells. See Table 90 below for lipid structures.

TABLE 80
Mean molecules of Cas9 protein per cell in macrophage cells
following editing with various lipid compositions relative to Lipid A,
1.5% 2kDMG PEG composition.

| LNP | | | Relative | |
|---|---|---|---|---|
| | PEG | | molecules/cell | |
| Ionizable lipid | lipid | Lipid composition | Mean | SD |
| Lipid A | 2kDMG | 50/10/38.5/1.5 | 1.00 | n/a |
| Lipid C | 2kDMG | 50/10/38.5/1.5 | 0.23 | 0.12 |
| Lipid D | 2kDMG | 50/10/38.5/1.5 | 2.14 | 0.10 |
| Lipid E | 2kDMG | 50/10/38.5/1.5 | 0.52 | 0.08 |
| Lipid F | 2kDMG | 50/10/38.5/1.5 | 1.10 | 0.07 |
| Lipid G | 2kDMG | 50/10/38.5/1.5 | 1.01 | 0.03 |
| Lipid A | 2kDMG | 50/9/38/3 | 0.88 | 0.43 |
| Lipid E | 2kDMG | 50/9/38/3 | 0.39 | 0.10 |
| Lipid G | 2kDMG | 50/9/38/3 | 0.99 | 0.22 |
| Lipid A | Lipid H | 50/10/38.5/1.5 | 2.43 | 0.91 |
| Lipid A | Lipid J | 50/10/38.5/1.5 | 2.12 | 0.37 |

### Example 31. Editing in Macrophages and Monocytes

To determine LNP editing efficiency, time of editing for monocyte-derived macrophage cells was examined. Surface expression of B2M protein was assessed with a guide targeting B2M either at the beginning of differentiation, thereby editing monocytes at Day 0, or towards the end of differentiation to macrophages at Day 5.

CD14+ cells were isolated from a leukopak obtained commercially (Hemacare) using StraightFrom® Leukopak® CD14 MicroBead Kit, human (Miltenyi Biotec, Catalog, 130-117-020) following the manufacturers protocol on MultiMACS™ Cell24 Separator Plus instrument. Following MACS isolation, CD14+ cells were cultured in triplicate at 100,000 cells/well in RPMI-1640 media with 10 ng/mL GM-CSF (Stemcell, 78140.1) on tissue culture plates (Falcon, 353072) for editing at Day 0 or non-tissue culture plates (Falcon, 351172) for editing at Day 5 post-CD14+ isolation. Due to the increased plate adherence of macrophages throughout differentiation and maturation process, non-tissue culture plates were used for macrophage samples for ease of detachment, which is needed for further flow analysis.

Cells were treated with LNPs delivering mRNA encoding Cas9 (SEQ ID NO. 6) sgRNA G00529 (SEQ ID NO: 701) targeting B2M the day of isolation or 5 days post-CD14+ cell isolation. LNPs were generally prepared as described in Example 1 with the lipid composition of 50/10/38.5/1.5, expressed as the molar ratio of ionizable lipid/cholesterol/DSPC/PEG, respectively. LNPs were serially diluted to final concentration range of 1.25-10 ug/mL and pre-incubated at 37° C. for 15 minutes with ApoE3 (Peprotech 350-02) at 10 ug/ml. The pre-incubated LNPs were added to cells at a total RNA cargo dose indicated in Table 81.

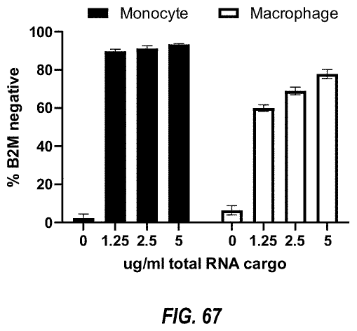

Five days post LNP treatment, cells were phenotyped by flow cytometry. Briefly, monocyte-derived macrophages were detached from culture plates by incubating the cells with 0.05% trypsin and 0.53 mM EDTA (Corning, 25-051-CI) at 37 C for 30 minutes or until cells are detached from the plate as visually determined by microscope verification. Detached cells were transferred to a new plate (Corning, 3799) and washed with PBS and media to inactivate the trypsin. Cells were further stained with LIVE/DEAD Violet (Life Technologies, L34955) in PBS for 15 minutes in the dark at room temperature. Cells were washed and incubated with antibodies targeting CD11b (Biolegend, 301306), B2M (Biolegend, 316312) and CD86 (Biolegend, 305420) for 30 minutes in dark on ice. Cells were washed and fixed and permeabilized by initially incubating cells with Reagent A (ThermoFisher, GAS001S100) for 20 minutes at room temperature followed by incubating cells with Reagent B (ThermoFisher, GAS002S100) containing antibody targeting CD68 (Biolegend, 333806) for 30 minutes at room temperature in dark. Cells were subsequently washed, resuspended in FACS buffer and processed on a Cytoflex instrument (Beckman Coulter) and analyzed using the FlowJo software package. Cells were gated on size, CD11b/CD68 positive status, and B2M negative population. Table 81 and FIG. 67 show increased percentage of B2M negative cell population in cells treated with LNPs, demonstrating editing is effective in both monocytes and macrophages. Editing is increased under these conditions in monocytes compared to macrophages. Editing in monocytes and macrophages was also observed when either cells or LNPs were prepared with serum.

TABLE 81
Mean B2M negative cells following editing with LNP

| Total Final | | | | |
|---|---|---|---|---|
| RNA | Monocyte | | Macrophage | |
| (ug/ml) | Mean | SD | Mean | SD |
| 0 | 2.27 | 2.20 | 6.36 | 2.43 |
| 1.25 | 89.71 | 1.17 | 60.02 | 1.68 |
| 2.5 | 91.23 | 1.50 | 68.99 | 2.00 |
| 5 | 93.34 | 0.41 | 77.84 | 2.36 |

### Example 32. Editing Time Course in Monocyte-Derived Macrophages Using Lipid Nanoparticles

In this study the editing efficiency at different days of monocyte differentiation into macrophages was monitored utilizing two serum-conditions (serum-free and 5% human serum, yielding to final 2.5% human serum concentration).

CD14+ cells were isolated as described in Example 31 and were frozen for later use. CD14+ cells were thawed and cultured in triplicate at 100,000 cells/well in OpTmizer base media as described in Table 2, 10 ng/mL GM-CSF (Stemcell, 78140.1) and with or without 2.5% human AB serum, on non-tissue culture plates (Falcon, 351172). Cells were treated with LNPs delivering Cas9 mRNA and gRNA G000529(SEQ ID NO: 701) targeting B2M at intervals ranging from the day of thaw through 8 days post-thaw and in either serum-free or final 2.5% human AB serum-containing media. LNPs were generally prepared as described in Example 1 with the lipid composition of 50/10/38.5/1.5, expressed as the molar ratio of ionizable lipid/cholesterol/DSPC/PEG, respectively. LNPs were pre-incubated at 37° C. for 15 minutes with ApoE3 (Peprotech 350-02) at 10 ug/ml. The pre-incubated LNPs were added to cells at 5 ug/ml total RNA cargo.

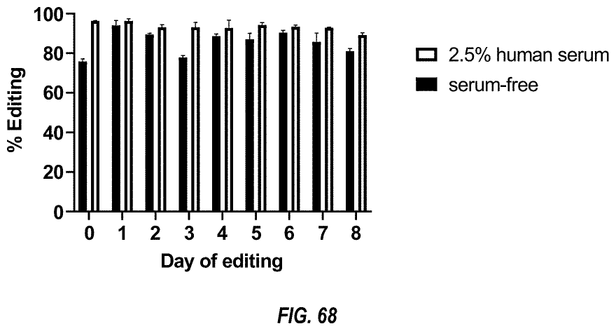

Six days post each LNP treatment, cells were collected and NGS analysis was performed as described in Example 1. As shown in Table 82 and FIG. 68, robust editing was achieved throughout macrophage differentiation and maturation with editing observed from zero to eight days post thaw of CD14+ cells. Editing was effective using media with or without human serum.

TABLE 82
Mean percent editing with LNP treatment at intervals post thaw

| | | | 2.5% Human | |
|---|---|---|---|---|
| Timepoint | Serum-free | | serum | |
| (days) | Mean | SD | Mean | SD |
| 0 | 75.80 | 1.31 | 96.27 | 0.21 |
| 1 | 94.03 | 2.47 | 96.27 | 1.10 |
| 2 | 89.43 | 0.64 | 93.10 | 1.35 |
| 3 | 77.90 | 0.99 | 93.13 | 2.41 |
| 4 | 88.63 | 1.07 | 92.80 | 3.91 |
| 5 | 87.03 | 3.00 | 94.17 | 1.31 |
| 6 | 90.40 | 1.15 | 93.33 | 0.97 |
| 7 | 85.73 | 4.50 | 92.90 | 0.26 |
| 8 | 81.00 | 1.37 | 89.17 | 1.11 |

### Example 33. Serial Editing in Macrophage Cell Using Lipid Nanoparticles

In this study serial editing capabilities in differentiating monocytes were demonstrated using LNPs with guides targeting CIITA or B2M. Isolated CD14+ monocytes were edited on Day 1 and Day 2 following thaw with either CIITA or B2M LNP formulations. Results were analyzed by flow cytometry.

CD14+ cells were isolated as described in Example 31 and were frozen for later use. At Day 0 of the study, frozen cells were thawed and cultured in triplicate wells at 100,000 cells/well in X-VIVO15 media with 10 ng/mL GM-CSF (Stemcell, 78140.1) on non-tissue culture plates (Falcon, 351172). Cells were treated with LNPs delivering mRNA encoding Cas9 (SEQ ID No. 6) and either sgRNA G000529 (SEQ ID NO: 701) targeting B2M or sgRNA G013674 (SEQ ID NO: 702) targeting CIITA on day after thaw as indicated in Table 83. Two days after thaw, cells were washed and treated with a second LNP delivering mRNA encoding Cas9 (SEQ ID NO. 6) and either sgRNA G000529 (SEQ ID NO: 701) targeting B2M or sgRNA G013674 (SEQ ID NO: 702) targeting CIITA as indicated in Table 83. LNPs were generally prepared as Example 1 with the lipid composition of 50/10/38.5/1.5, expressed as the molar ratio of ionizable lipid/cholesterol/DSPC/PEG, respectively. LNPs at 5 ug/mL total RNA cargo were preincubated at 37° C. for 15 minutes with 10 ug/ml ApoE3 (Peprotech 350-02) in either serum-free or 5% human serum media. The pre-incubated LNPs were added to cells yielding a final total RNA cargo concentration of 2.5 ug/mL and final serum concentrations of 0% or 2.5%.

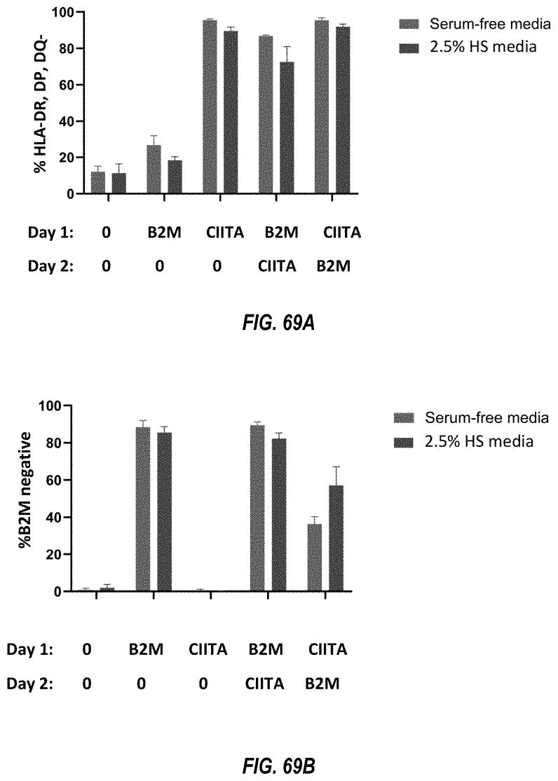

Eight days post thaw, cells were phenotyped by flow cytometry. Briefly, monocyte-derived macrophages were detached from culture plates by incubating the cells with 0.05% trypsin and 0.53 mM EDTA (Corning, 25-051-CI) at 37 C for 30 minutes or until cells are detached from the plate as determined by microscope verification. Detached cells were transferred to a new plate (Corning, 3799) and washed to inactivate the trypsin. Cells were incubated with antibodies targeting CD11b (Biolegend, 301306), B2M (Biolegend, 316312) and HLA-DR, DP, DQ (Biolegend, 361706) for 30 minutes in dark, on ice. Cells were subsequently washed, processed on a Cytoflex instrument (Beckman Coulter) and analyzed using the FlowJo software package. Cells were gated on size, viability, CD11b+ population followed by B2M negative or HLA-DR, DP, DQ negative population. Cytometry data is shown in Table 83 and FIGS. 69A-B. Dual editing was successful as both substantial populations of HLA-DR, DP, DQ negative and of B2M negative cells were observed following editing with two LNPs in both serum-free and 5% human serum media conditions.

TABLE 83
Mean percent B2M negative cells or HLA-DR, DP, DQ
negative cells following with serial LNP treatment

| | | | % B2M | | % HLA-DR, DP, | |
|---|---|---|---|---|---|---|
| | Day 1 | Day 2 | negative | | DQ negative | |
| Serum | edit | edit | Mean | SD | Mean | SD |
| Serum | 0 | 0 | 1.95 | 1.84 | 11.28 | 5.12 |
| free | B2M | 0 | 85.56 | 3.18 | 18.36 | 1.94 |
| media | CIITA | 0 | 0.22 | 0.15 | 89.56 | 2.18 |
| | B2M | CIITA | 82.27 | 3.00 | 72.60 | 8.33 |
| | CIITA | B2M | 57.10 | 10.06 | 91.85 | 1.53 |
| 2.5% | 0 | 0 | 0.51 | 0.52 | 8.16 | 2.05 |
| human | B2M | 0 | 81.42 | 0.87 | 28.75 | 9.31 |
| serum | CIITA | 0 | 0.00 | 0.00 | 77.75 | 4.39 |
| | B2M | CIITA | 86.77 | 1.65 | 54.88 | 3.62 |
| | CIITA | B2M | 63.56 | 2.93 | 69.04 | 4.44 |

### Example 34. Editing in Monocytes and Macrophages with Select Ionizable Lipids

To assess editing efficacy using LNPs formulated with selected ionizable lipids, editing was assessed by NGS and flow cytometry in monocytes and macrophages.

CD14+ cells were isolated as in Example 31 and frozen for future use. CD14+ cells were thawed and cultured in triplicate at 100,000 cells/well in OpTmizer base media as described in Table 2 with 10 ng/mL GM-CSF (Stemcell, 78140.1) on non-tissue culture plates (Falcon, 351172) for ease of detachment. Cells were treated with LNPs delivering mRNA encoding Cas9 (SEQ ID NO. 6) and gRNA G000529 (SEQ ID NO. 701) targeting B2M. For monocytes, LNP addition occurred on the same day as plating on tissue culture plates. For macrophages, LNP addition occurred after 5 days incubation on non-tissue culture plates (Falcon, 351172). LNPs were generally prepared as Example 1 using the ionizable lipids indicated in Tables 84 and 85 with the lipid composition of 50/10/38.5/1.5, expressed as the molar ratio of ionizable lipid/cholesterol/DSPC/PEG, respectively. LNPs were preincubated at 37° C. for 15 minutes with ApoE3 (Peprotech 350-02) at 10 ug/ml. The pre-incubated LNPs were added to cells in 1:1 v/v ratio, yielding a final total RNA cargo dose of 2.5 ug/mL or 5 ug/mL.

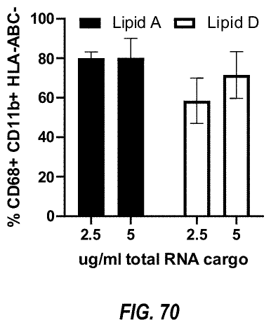

Six days post LNP treatment, monocyte-engineered cells were phenotyped by flow cytometry and both monocyte and macrophage-engineered cells were collected for NGS as described in Example 1. Briefly, cells were incubated with antibodies targeting CD68, CD11b and HLA-ABC (Biolegend, 311432) as generally as described in Example 31. HLA-ABC targeting antibody was used in place of B2M. Cells were subsequently washed, processed on a Cytoflex instrument (Beckman Coulter) and analyzed using the FlowJo software package. Cells were gated on size, CD68+, CD11b+ followed by HLA-ABC− population. Cytometry data is shown in Table 84 and FIG. 70. NGS editing data is shown is Table 85. Both Lipid A and Lipid D formulations showed effective editing at Day 0 and Day 5 post thaw. Editing was also observed when cells were cultured in RPMI or XVIVO-15 media.

TABLE 84
Mean percentage of cells displaying surface protein knockout in
cells after editing monocytes with LNPs with varied ionizable lipids

| | | % CD68 + CD11b + | |
|---|---|---|---|
| Ionizable | Total RNA | HLA-ABC− | |
| lipid | (ug/ml) | Mean | SD |
| Unedited | 0 | 3.07 | 1.02 |
| Lipid A | 2.5 | 80.08 | 3.14 |
| Lipid A | 5 | 80.23 | 9.81 |
| Lipid D | 2.5 | 58.48 | 11.52 |
| Lipid D | 5 | 71.57 | 11.82 |

TABLE 85
Mean percentage editing in cells after treatment with LNPs with
varied ionizable lipids

| Ionizable | Total RNA | Day 0 Edit | | Day 5 Edit | |
|---|---|---|---|---|---|
| lipid | (ug/mL) | Mean | SD | Mean | SD |
| Lipid A | 2.5 | 85.70 | 3.51 | 93.13 | 3.15 |
| Lipid A | 5 | 87.03 | 5.14 | 96.90 | 0.42 |
| Lipid D | 2.5 | 81.77 | 1.50 | 77.77 | 3.23 |
| Lipid D | 5 | 89.37 | 0.50 | 84.13 | 1.61 |

### Example 35. Editing in iPSC Using Lipid Nanoparticles

To determine editing efficacy via LNPs, induced pluripotent stem cells (iPSCs) are treated with LNPs delivering mRNA encoding Cas9 and a sgRNA targeting B2M.

Human iPSC cells edited at the TRBC1/2 loci are obtained commercially. TRBC-edited cells are thawed, washed and resuspend in media. Cells are cultured on Geltrex (Thermo Fisher) coated plates with media refreshed daily. Five days post-thaw, iPSC cells are dissociated and replated to a Geltrex-coated 96-well plate. Twenty-four hours after replating, cells are washed and resuspended in media. LNPs delivering mRNA encoding Cas9 and a sgRNA targeting B2M are prepared by preincubating with ApoE3 at 37 C for 15 minutes. The pre-incubated LNP mixture is transferred to iPSC cells. Cells are washed 24 hours after initial LNP exposure and media is refreshed daily thereafter. Cells are collected 3, 5 and 7 days after LNP editing and analyzed by NGS for genome editing at the B2M locus.

### Example 36. LNP Composition Activity Evaluated in Serum Media Conditions

To evaluate LNP editing efficacy, LNP compositions were evaluated the effect of alternative media conditions on insertion efficiency in CD3 positive T cells. T cells were treated with LNP compositions with varied molar ratios of lipid components encapsulating Cas9 mRNA and a sgRNA targeting the TRAC gene. An AAV6 viral construct delivered a homology directed repair template (HDRT) that encoded a GFP reporter flanked by homology arms for site-specific integration into the TRAC locus (Vigene; SEQ ID NO: 8). TRAC gene disruption was assessed by flow cytometry for loss of T cell receptor surface proteins. Insertion was assessed by flow cytometry for GFP luminescence.

LNPs were generally prepared as described in Example 1 with the lipid composition as indicated in Table 86, expressed as the molar ratio of ionizable lipid A/cholesterol/DSPC/PEG, respectively. LNP delivered mRNA encoding Cas9 (SEQ ID NO. 6) and sgRNA G013006 targeting human TRAC. The cargo ratio of sgRNA to Cas9 mRNA was 1:2 by weight. LNPs were preincubated with ApoE3 as in Example 21.

T cells from a single donor were prepared as described in Example 21 with the following media modifications. T cells were plated with media supplemented with either 2.5% human AB serum (HABS), 2.5% CTS Immune Cell SR (Gibco, Cat #A25961-01) serum replacement (SR), 5% serum replacement (SR), or the combination of 2.5% human AB serum and 2.5% serum replacement. T cells were activated 24 hours post thaw as described in Example 21. Two days post activation, T cells were transfected with LNPs as described in Example 21 at LNP concentrations of 0.31 μg/ml, 0.63 μg/ml, 1.25 μg/ml, and 2.5 μg/ml. AAV6 encoding a GFP reporter flanked by homology arms for site-specific integration into the TRAC locus (Vigene; SEQ ID NO: 8) was added to each well at a multiplicity of infection (MOI) of 3×10e5 viral particles/cell. Compound 4, a small molecule inhibitor of DNA-dependent protein kinase, was added at 0.25 uM. After 24 hours, all cells were split into media containing 5% HABS.

Five days post transfection, T cells were phenotyped by flow cytometry analysis as described in Example 21 to evaluate the insertion efficiency of the LNP compositions. Table 86 shows the percent of CD3 negative cells. The T cell receptor alpha chain encoded by TRAC is required for T cell receptor/CD3 complex assembly and translocation to the cell surface. Accordingly, disruption of the TRAC gene by genome editing leads to a loss of CD3 protein on the cell surface of T cells. The mean percentage of GFP positive T cells for each media condition is shown in Table 87. Cells expressing GFP protein indicate successful insertion into genome.

TABLE 86
Percent CD3 negative T cells following treatment of activated
T cells with AAV and indicated LNP formulation.

| | | | | | | | | 2.5% | |
|---|---|---|---|---|---|---|---|---|---|
| Com- | LNP | 2.5% | | | | | | HABS & | |
| po- | (ug/ | HABS | | 2.5% SR | | 5% SR | | 2.5% SR | |
| sition | ml) | Mean | SD | Mean | SD | Mean | SD | Mean | SD |
| 50/10/ | 2.5 | 94.55 | 0.07 | 99.00 | 0.14 | 97.95 | 0.07 | 99.25 | 0.07 |
| 38.5/ | 1.25 | 92.25 | 0.35 | 96.05 | 1.20 | 92.10 | 2.55 | 95.95 | 0.21 |
| 1.5 | 0.63 | 76.55 | 0.21 | 76.60 | 3.11 | 63.55 | 2.47 | 74.70 | 1.27 |
| | 0.31 | 48.35 | 1.34 | 25.95 | 1.20 | 16.55 | 2.47 | 41.55 | 1.20 |
| 35/15/ | 2.5 | 99.40 | 0.00 | 98.80 | 0.42 | 98.65 | 0.07 | 98.70 | 0.14 |
| 47.5/ | 1.25 | 98.85 | 0.07 | 98.95 | 0.64 | 98.50 | 0.14 | 97.25 | 0.35 |
| 2.5 | 0.63 | 94.10 | 0.28 | 96.80 | 0.42 | 95.25 | 0.35 | 84.60 | 1.41 |
| | 0.31 | 75.20 | 0.14 | 68.75 | 9.97 | 64.20 | 0.57 | 50.30 | 1.56 |

TABLE 87
Percent GFP+ cells following treatment of activated T cells
with AAV and indicated LNP formulations.

| | LNP | 2.5% | | | | | | 2.5% HABS | |
|---|---|---|---|---|---|---|---|---|---|
| Com- | (ug/ | HABS | | 2.5% SR | | 5% SR | | and 2.5% SR | |
| position | mL) | Mean | SD | Mean | SD | Mean | SD | Mean | SD |
| 50/10/ | 2.5 | 94.6 | 0.0 | 95.4 | 0.4 | 93.7 | 0.6 | 90.0 | 0.1 |
| 38.5/1.5 | 1.25 | 92.3 | 0.3 | 92.3 | 0.5 | 87.7 | 1.8 | 75.1 | 1.1 |
| | 0.63 | 76.6 | 0.1 | 76.3 | 1.3 | 63.1 | 1.8 | 31.2 | 2.6 |
| | 0.31 | 48.4 | 0.9 | 30.5 | 1.0 | 19.5 | 2.5 | 5.0 | 0.7 |
| 35/15/ | 2.5 | 95.6 | 0.0 | 96.3 | 0.5 | 95.6 | 0.1 | 94.7 | 0.4 |
| 47.5/2.5 | 1.25 | 94.8 | 0.5 | 96.0 | 1.4 | 95.6 | 0.0 | 91.1 | 0.2 |
| | 0.63 | 89.6 | 0.6 | 93.3 | 0.5 | 91.1 | 0.2 | 78.8 | 1.0 |
| | 0.31 | 74.7 | 0.1 | 75.0 | 0.0 | 64.4 | 0.4 | 48.8 | 1.2 |

### Example 37. Editing iPSC with Lipid Nanoparticles

To determine editing efficacy via LNPs, induced pluripotent stem cells (iPSCs) were treated with LNPs delivering mRNA encoding Cas9 and a sgRNA targeting B2M.

Human iPSC cells (Alstem, iPS11) edited at the TRBC1 and TRBC2 loci by electroporation with Cas9 RNP using guide G014832 (SEQ ID NO: 723) were produced by the Centre for Commercialization of Regenerative Medicine. Geltrex (Thermo Fisher, A1413302) was thawed overnight, 1:100 diluted with DMEM/F-12 (Thermo Fisher, 11330032) and applied at 1 mL/well of a 6-well plate (Falcon, 140675). The Geltrex treated plate was incubated at 37° C. for 1 hour and washed prior to use.

TRBC-edited iPSCs were thawed, washed, and resuspended in room temperature Essential 8 (E8) media (Themo Fisher, A1517001), and cultured at 37° C. in two wells of Geltrex-coated 6-well plates. Media was refreshed daily with room temperature E8.

Five days post-thaw, cell media was refreshed three hours prior to splitting cells. iPSCs were washed with 2 mL/well PBS (Corning, 21-040-CM). Gentle Cell Dissociation Reagent (StemCell Technologies, 07174) was added at 0.5 mL/well and distributed evenly. Cells were incubated at 37° C. for 12 minutes, the plate was tapped firmly to dissociate cells, and cells were resuspended in 1 mL of room temperature E8 media. The cells were collected from the plate by pipetting up and down. An additional 1 mL media was used to wash the plate and recover all the cells. Total cell count was obtained by hemacytometer. A 96-well plate (Thermo Fisher, 353072) was prepared as above using 60 uL/well diluted Ggeltrex. Cells were centrifuged at 200G for 3 minutes and plated to the Geltrex-coated 96-well plate at a cell density of 15,000 cells/well in 80 uL room temperature E8 media with 10 uM Rock inhibitor Y-27632 dihydrochloride (Tocris, 1254). After twenty-four hours incubation at 37° C., cells were washed and resuspended in 40 uL room temperature E8 media.

Cells were treated with LNPs delivering mRNA encoding Cas9 (SEQ ID NO. 6) and gRNA G000529 (SEQ ID NO. 701) targeting B2M. LNPs were generally prepared as Example 1 with the lipid composition of 50/10/38.5/1.5, expressed as the molar ratio of ionizable lipid/cholesterol/DSPC/PEG, respectively. LNPs were prepared with a ratio of gRNA to mRNA of 1:2 by weight. LNPs were preincubated at 5 ug/mL with 10 ug/mL ApoE3 at 37 C for 15 minutes. LNP mixture was transferred to iPSC cells yielding a final dose 2.5 ug/mL total RNA cargo per well. Cells were washed 24 hours after LNP addition and media was refreshed daily thereafter. Cells were harvested 3, 5, and 7 days after LNP addition and analyzed by NGS as described in Example 1. Mean editing percentage in iPSC following LNP treatment in shown in Table 88.

TABLE 88
Percent editing at indicated timepoint following iPSC
treatment with LNP.

| | Unedited cells | | 2.5 ug/mL LNP | | |
|---|---|---|---|---|---|
| Day | Mean | SD | Mean | SD | N |
| Day 3 | 0.07 | 0.06 | 96.03 | 1.46 | 3 |
| Day 5 | 0.03 | 0.06 | 95.10 | 1.47 | 3 |
| Day 7 | 0.20 | 0.26 | 98.60 | 0.44 | 3 |

In the following table and throughout, the terms “mA,” “mC,” “mU,” or “mG” are used to denote a nucleotide that has been modified with 2′-O-Me.

In the following table, a “*” is used to depict a PS modification. In this application, the terms A*, C*, U*, or G* may be used to denote a nucleotide that is linked to the next (e.g., 3′) nucleotide with a PS bond.

It is understood that if a DNA sequence (comprising Ts) is referenced with respect to an RNA, then Ts should be replaced with Us (which may be modified or unmodified depending on the context), and vice versa.

In the following table, single amino acid letter code is used to provide peptide sequences.

TABLE 89
List of sequences

| | SEQ ID | |
|---|---|---|
| Description | NO | Sequence |
| gRNA | 701 | mG*mG*mC*CACGGAGCGAGACAUCUGUUUUAGAmGmCmUmAmGm |
| G000529 | | AmAmAmUmAmGmCAAGUUAAAAUAAGGCUAGUCCGUUAUCAmAm |
| | | CmUmUmGmAmAmAmAmAmGmUmGmGmCmAmCmCmGmAmGmUmC |
| | | mGmGmUmGmCmU*mU*mU*mU |
| gRNA | 702 | mU*mU*mC*UAGGGGCCCCAACUCCAGUUUUAGAmGmCmUmAmGm |
| G013674 | | AmAmAmUmAmGmCAAGUUAAAAUAAGGCUAGUCCGUUAUCAmAm |
| | | CmUmUmGmAmAmAmAmAmGmUmGmGmCmAmCmCmGmAmGmUmC |
| | | mGmGmUmGmCmU*mU*mU*mU |
| gRNA | 703 | mA*mG*mA*GUCUCUCAGCUGGUACAGUUUUAGAmGmCmUmAmGm |
| G012086 | | AmAmAmUmAmGmCAAGUUAAAAUAAGGCUAGUCCGUUAUCAmAm |
| | | CmUmUmGmAmAmAmAmAmGmUmGmGmCmAmCmCmGmAmGmUmC |
| | | mGmGmUmGmCmU*mU*mU*mU |
| gRNA | 707 | mG*mG*mC*CUCGGCGCUGACGAUCUGUUUUAGAmGmCmUmAmGm |
| G016239 | | AmAmAmUmAmGmCAAGUUAAAAUAAGGCUAGUCCGUUAUCAmAm |
| | | CmUmUmGmAmAmAmAmAmGmUmGmGmCmAmCmCmGmAmGmUmC |
| | | mGmGmUmGmCmU*mU*mU*mU |
| gRNA | 708 | mC*mU*mC*UCAGCUGGUACACGGCAGUUUUAGAmGmCmUmAmGm |
| G013006 | | AmAmAmUmAmGmCAAGUUAAAAUAAGGCUAGUCCGUUAUCAmAm |
| | | CmUmUmGmAmAmAmAmAmGmUmGmGmCmAmCmCmGmAmGmUmC |
| | | mGmGmUmGmCmU*mU*mU*mU |
| gRNA | 709 | mG*mG*mC*CACGGAGCGAGACAUCUGUUUUAGAmGmCmUmAmGm |
| G012738 | | AmAmAmUmAmGmCAAGUUAAAAUAAGGCUAGUCCGUUAUCAmAm |
| | | CmUmUmGmAmAmAmAmAmGmUmGmGmCmAmCmCmGmAmGmUmC |
| | | mGmGmUmGmCmU*mU*mU*mU |
| gRNA | 710 | mC*mC*mA*AUAUCAGGAGACUAGGAGUUUUAGAmGmCmUmAmGm |
| G000562 | | AmAmAmUmAmGmCAAGUUAAAAUAAGGCUAGUCCGUUAUCAmAm |
| | | CmUmUmGmAmAmAmAmAmGmUmGmGmCmAmCmCmGmAmGmUmC |
| | | mGmGmUmGmCmU*mU*mU*mU |
| gRNA | 711 | mU*mU*mA*CCCCACUUAACUAUCUUGUUUUAGAmGmCmUmAmGm |
| G015995 | | AmAmAmUmAmGmCAAGUUAAAAUAAGGCUAGUCCGUUAUCAmAm |
| | | CmUmUmGmAmAmAmAmAmGmUmGmGmCmAmCmCmGmAmGmUmC |
| | | mGmGmUmGmCmU*mU*mU*mU |
| gRNA | 712 | mC*mC*mA*CUCUGCCCCAUGGGCUCGUUUUAGAmGmCmUmAmGm |
| G016017 | | AmAmAmUmAmGmCAAGUUAAAAUAAGGCUAGUCCGUUAUCAmAm |
| | | CmUmUmGmAmAmAmAmAmGmUmGmGmCmAmCmCmGmAmGmUmC |
| | | mGmGmUmGmCmU*mU*mU*mU |
| gRNA | 713 | mC*mG*mC*UGUCAAGUCCAGUUCUAGUUUUAGAmGmCmUmAmGm |
| G016206 | | AmAmAmUmAmGmCAAGUUAAAAUAAGGCUAGUCCGUUAUCAmAm |
| | | CmUmUmGmAmAmAmAmAmGmUmGmGmCmAmCmCmGmAmGmUmC |
| | | mGmGmUmGmCmU*mU*mU*mU |
| gRNA | 714 | mG*mC*mG*UCCACAUCCUGCAAGGGGUUUUAGAmGmCmUmAmGm |
| G018117 | | AmAmAmUmAmGmCAAGUUAAAAUAAGGCUAGUCCGUUAUCAmAm |
| | | CmUmUmGmAmAmAmAmAmGmUmGmGmCmAmCmCmGmAmGmUmC |
| | | mGmGmUmGmCmU*mU*mU*mU |
| gRNA | 715 | mU*mG*mG*UCAGGGCAAGAGCUAUUGUUUUAGAmGmCmUmAmGm |
| G013676 | | AmAmAmUmAmGmCAAGUUAAAAUAAGGCUAGUCCGUUAUCAmAm |
| | | CmUmUmGmAmAmAmAmAmGmUmGmGmCmAmCmCmGmAmGmUmC |
| | | mGmGmUmGmCmU*mU*mU*mU |
| gRNA | 716 | mA*mC*mA*GCGACGCCGCGAGCCAGGUUUUAGAmGmCmUmAmGm |
| G018995 | | AmAmAmUmAmGmCAAGUUAAAAUAAGGCUAGUCCGUUAUCAmAm |
| | | CmUmUmGmAmAmAmAmAmGmUmGmGmCmAmCmCmGmAmGmUmC |
| | | mGmGmUmGmCmU*mU*mU*mU |
| pINT1405, | 717 | ttggccactccctctctgcgcgctcgctcgctcactgaggccgggcgaccaaaggtcgcccgacgcccgggcttt |
| HD1TCR | | gcccgggcggcctcagtgagcgagcgagcgcgcagagagggagtggccaactccatcactaggggttcctag |
| insertion | | atcttgccaacataccataaacctcccattctgctaatgcccagcctaagttggggagaccactccagattccaagat |
| including | | gtacagtttgctttgctgggcctttttcccatgcctgcctttactctgccagagttatattgctggggttttgaagaagat |
| ITRs | | cctattaaataaaagaataagcagtattattaagtagccctgcatttcaggtttccttgagtggcaggccaggcctgg |
| | | ccgtgaacgttcactgaaatcatggcctcttggccaagattgatagcttgtgcctgtccctgagtcccagtccatcac |
| | | gagcagctggtttctaagatgctatttcccgtataaagcatgagaccgtgacttgccagccccacagagccccgcc |
| | | cttgtccatcactggcatctggactccagcctgggttggggcaaagagggaaatgagatcatgtcctaaccctgat |
| | | cctcttgtcccacagatatccagaaccctgaccctgcggctccggtgcccgtcagtgggcagagcgcacatcgcc |
| | | cacagtccccgagaagttggggggaggggtcggcaattgaaccggtgcctagagaaggtggcgcggggtaaa |
| | | ctgggaaagtgatgtcgtgtactggctccgcctttttcccgagggtgggggagaaccgtatataagtgcagtagtc |
| | | gccgtgaacgttctttttcgcaacgggtttgccgccagaacacaggtaagtgccgtgtgtggttcccgcgggcctg |
| | | gcctctttacgggttatggcccttgcgtgccttgaattacttccacgcccctggctgcagtacgtgattcttgatcccg |
| | | agcttcgggttggaagtgggtgggagagttcgaggccttgcgcttaaggagccccttcgcctcgtgcttgagttga |
| | | ggcctggcttgggcgctggggccgccgcgtgcgaatctggtggcaccttcgcgcctgtctcgctgctttcgataag |
| | | tctctagccatttaaaatttttgatgacctgctgcgacgctttttttctggcaagatagtcttgtaaatgcgggccaagat |
| | | gtgcacactggtatttcggtttttggggccgcgggcggcgacggggcccgtgcgtcccagcgcacatgttcggcg |
| | | aggcggggcctgcgagcgcggccaccgagaatcggacgggggtagtctcaagctggccggcctgctctggtg |
| | | cctggcctcgcgccgccgtgtatcgccccgccctgggcggcaaggctggcccggtcggcaccagttgcgtgag |
| | | cggaaagatggccgcttcccggccctgctgcagggagctcaaaatggaggacgcggcgctcgggagagcggg |
| | | cgggtgagtcacccacacaaaggaaaagggcctttccgtcctcagccgtcgcttcatgtgactccacggagtacc |
| | | gggcgccgtccaggcacctcgattagttctcgagcttttggagtacgtcgtctttaggttggggggaggggttttatg |
| | | cgatggagtttccccacactgagtgggtggagactgaagttaggccagcttggcacttgatgtaattctccttggaat |
| | | ttgccctttttgagtttggatcttggttcattctcaagcctcagacagtggttcaaagtttttttcttccatttcaggtgt |
| | | cgtgatgcggccgccaccatgggatcttggacactgtgttgcgtgtccctgtgcatcctggtggccaagcacacagatg |
| | | ccggcgtgatccagtctcctagacacgaagtgaccgagatgggccaagaagtgaccctgcgctgcaagcctatc |
| | | agcggccacgattacctgttctggtacagacagaccatgatgagaggcctggaactgctgatctacttcaacaaca |
| | | acgtgcccatcgacgacagcggcatgcccgaggatagattcagcgccaagatgcccaacgccagcttcagcac |
| | | cctgaagatccagcctagcgagcccagagatagcgccgtgtacttctgcgccagcagaaagacaggcggctac |
| | | agcaatcagccccagcactttggagatggcacccggctgagcatcctggaagatctgaagaacgtgttcccacct |
| | | gaggtggccgtgttcgagccttctgaggccgagatcagccacacacagaaagccacactcgtgtgtctggccac |
| | | cggcttctatcccgatcacgtggaactgtcttggtgggtcaacggcaaagaggtgcacagcggcgtcagcaccga |
| | | tcctcagcctctgaaagagcagcccgctctgaacgacagcagatactgcctgagcagcagactgagagtgtccg |
| | | ccaccttctggcagaaccccagaaaccacttcagatgccaggtgcagttctacggcctgagcgagaacgatgagt |
| | | ggacccaggatagagccaagcctgtgacacagatcgtgtctgccgaagcctggggcagagccgattgtggcttt |
| | | accagcgagagctaccagcagggcgtgctgtctgccacaatcctgtacgagatcctgctgggcaaagccactctg |
| | | tacgccgtgctggtgtctgccctggtgctgatggccatggtcaagcggaaggatagcaggggcggctccggtgc |
| | | cacaaacttctccctgctcaagcaggccggagatgtggaagagaaccctggccctatggaaaccctgctgaaggt |
| | | gctgagcggcacactgctgtggcagctgacatgggtccgatctcagcagcctgtgcagtctcctcaggccgtgatt |
| | | ctgagagaaggcgaggacgccgtgatcaactgcagcagctctaaggccctgtacagcgtgcactggtacagaca |
| | | gaagcacggcgaggcccctgtgttcctgatgatcctgctgaaaggcggcgagcagaagggccacgagaagatc |
| | | agcgccagcttcaacgagaagaagcagcagtccagcctgtacctgacagccagccagctgagctacagcggca |
| | | cctacttttgtggcaccgcctggatcaacgactacaagctgtctttcggagccggcaccacagtgacagtgcgggc |
| | | caatattcagaaccccgatcctgccgtgtaccagctgagagacagcaagagcagcgacaagagcgtgtgcctgtt |
| | | caccgacttcgacagccagaccaacgtgtcccagagcaaggacagcgacgtgtacatcaccgataagactgtgc |
| | | tggacatgcggagcatggacttcaagagcaacagcgccgtggcctggtccaacaagagcgatttcgcctgcgcc |
| | | aacgccttcaacaacagcattatccccgaggacacattcttcccaagtcctgagagcagctgcgacgtgaagctg |
| | | gtggaaaagagcttcgagacagacaccaacctgaacttccagaacctgagcgtgatcggcttcagaatcctgctg |
| | | ctcaaggtggccggcttcaacctgctgatgaccctgagactgtggtccagctaacctCGACTGTGCCTT |
| | | CTAGTTGCCAGCCATCTGTTGTTTGCCCCTCCCCCGTGCCTTCCTTGA |
| | | CCCTGGAAGGTGCCACTCCCACTGTCCTTTCCTAATAAAATGAGGAA |
| | | ATTGCATCGCATTGTCTGAGTAGGTGTCATTCTATTCTGGGGGGTGG |
| | | GGTGGGGCAGGACAGCAAGGGGGAGGATTGGGAAGACAATAGCAG |
| | | GCATGCTGGGGATGCGGTGGGCTCTATGGcttctgaggcggaaagaaccagctgggg |
| | | ctctagggggtatccccactagtcgtgtaccagctgagagactctaaatccagtgacaagtctgtctgcctattcacc |
| | | gattttgattctcaaacaaatgtgtcacaaagtaaggattctgatgtgtatatcacagacaaaactgtgctagacatga |
| | | ggtctatggacttcaagagcaacagtgctgtggcctggagcaacaaatctgactttgcatgtgcaaacgccttcaac |
| | | aacagcattattccagaagacaccttcttccccagcccaggtaagggcagctttggtgccttcgcaggctgtttcctt |
| | | gcttcaggaatggccaggttctgcccagagctctggtcaatgatgtctaaaactcctctgattggtggtctcggcctt |
| | | atccattgccaccaaaaccctctttttactaagaaacagtgagccttgttctggcagtccagagaatgacacgggaa |
| | | aaaagcagatgaagagaaggtggcaggagagggcacgtggcccagcctcagtctctagatctaggaaccccta |
| | | gtgatggagttggccactccctctctgcgcgctcgctcgctcactgaggccgcccgggcaaagcccgggcgtcg |
| | | ggcgacctttggtcgcccggcctcagtgagcgagcgagcgcgcagagagggagtggccaa |
| gRNA | 718 | mC*mC*mA*CACCCAAAAGGCCACACGUUUUAGAmGmCmUmAmGm |
| G016200 | | AmAmAmUmAmGmCAAGUUAAAAUAAGGCUAGUCCGUUAUCAmAm |
| | | CmUmUmGmAmAmAmAmAmGmUmGmGmCmAmCmCmGmAmGmUmC |
| | | mGmGmUmGmCmU*mU*mU*mU |
| gRNA | 719 | mC*mG*mC*CCAGGUCCUCACGUCUGGUUUUAGAmGmCmUmAmGm |
| G016086 | | AmAmAmUmAmGmCAAGUUAAAAUAAGGCUAGUCCGUUAUCAmAm |
| | | CmUmUmGmAmAmAmAmAmGmUmGmGmCmAmCmCmGmAmGmUmC |
| | | mGmGmUmGmCmU*mU*mU*mU |
| AAV6-1008 | 720 | tgcatcatcaccgtttttctggacaaccccaaagtaccccgtctccctggctttagccacctctccatcctcttgctttct |
| GFP insert | | ttgcctggacaccccgttctcctgtggattcgggtcacctctcactcctttcatttgggcagctcccctaccccccttac |
| for AAVS1 | | ctctctagtctgtgctagctcttccagccccctgtcatggcatcttccaggggtccgagagctcagctagtcttcttcct |
| | | ccaacccgggcccctatgtccacttcaggacagcatgtttgctgcctccagggatcctgtgtccccgagctgggac |
| | | caccttatattcccagggccggttaatgtggctctggttctgggtacttttatctgtcccctccaccccacagtggggc |
| | | cactagggacaggattggtgacagaaaagccccatccttaggcctcctccttccgagtaattcatacaaaaggact |
| | | cgcccctgccttggggaatcccagggaccgtcgttaaactcccactaacgtagaacccagagatcgctgcgttcc |
| | | cgccccctcacccgcccgctctcgtcatcactgaggtggagaagagcatgcgtgaggctccggtgcccgtcagt |
| | | gggcagagcgcacatcgcccacagtccccgagaagttggggggaggggtcggcaattgaaccggtgcctaga |
| | | gaaggtggcgcggggtaaactgggaaagtgatgtcgtgtactggctccgcctttttcccgagggtgggggagaa |
| | | ccgtatataagtgcagtagtcgccgtgaacgttctttttcgcaacgggtttgccgccagaacacaggtaagtgccgt |
| | | gtgtggttcccgcgggcctggcctctttacgggttatggcccttgcgtgccttgaattacttccacgcccctggctgc |
| | | agtacgtgattcttgatcccgagcttcgggttggaagtgggtgggagagttcgaggccttgcgcttaaggagcccc |
| | | ttcgcctcgtgcttgagttgaggcctggcttgggcgctggggccgccgcgtgcgaatctggtggcaccttcgcgc |
| | | ctgtctcgctgctttcgataagtctctagccatttaaaatttttgatgacctgctgcgacgctttttttctggcaagatagt |
| | | cttgtaaatgcgggccaacatctgcacactggtatttcggtttttggggccgcgggcggcgacggggcccgtgcgt |
| | | cccagcgcacatgttcggcgaggcggggcctgcgagcgcggccaccgagaatcggacgggggtagtctcaag |
| | | ctggccggcctgctctggtgcctggcctcgcgccgccgtgtatcgccccgccctgggcggcaaggctggcccg |
| | | gtcggcaccagttgcgtgagcggaaagatggccgcttcccggccctgctgcagggagctcaaaatggaggacg |
| | | cggcgctcgggagagcgggcgggtgagtcacccacacaaaggaaaagggcctttccgtcctcagccgtcgctt |
| | | catgtgactccacggagtaccgggcgccgtccaggcacctcgattagttctcgagcttttggagtacgtcgtcttta |
| | | ggttggggggaggggttttatgcgatggagtttccccacactgagtgggtggagactgaagttaggccagcttgg |
| | | cacttgatgtaattctccttggaatttgccctttttgagtttggatcttggttcattctcaagcctcagacagtggttcaaa |
| | | gtttttttcttccatttcaggtgtcgtgacgctagcgctaccggactcaatctcgagctcaagcttcgaattctgcagtc |
| | | gacggtaccgcgggcccgggatccaccggtcgccaccatggtgAGCAAGGGCGAGGAGCTG |
| | | TTCACCGGGGTGGTGCCCATCCTGGTCGAGCTGGACGGCGACGTAAA |
| | | CGGCCACAAGTTCAGCGTGTCCGGCGAGGGCGAGGGCGATGCCACC |
| | | TACGGCAAGCTGACCCTGAAGTTCATCTGCACCACCGGCAAGCTGCC |
| | | CGTGCCCTGGCCCACCCTCGTGACCACCCTGACCTACGGCGTGCAGT |
| | | GCTTCAGCCGCTACCCCGACCACATGAAGCAGCACGACTTCTTCAAG |
| | | TCCGCCATGCCCGAAGGCTACGTCCAGGAGCGCACCATCTTCTTCAA |
| | | GGACGACGGCAACTACAAGACCCGCGCCGAGGTGAAGTTCGAGGGC |
| | | GACACCCTGGTGAACCGCATCGAGCTGAAGGGCATCGACTTCAAGG |
| | | AGGACGGCAACATCCTGGGGCACAAGCTGGAGTACAACTACAACAG |
| | | CCACAACGTCTATATCATGGCCGACAAGCAGAAGAACGGCATCAAG |
| | | GTGAACTTCAAGATCCGCCACAACATCGAGGACGGCAGCGTGCAGC |
| | | TCGCCGACCACTACCAGCAGAACACCCCCATCGGCGACGGCCCCGTG |
| | | CTGCTGCCCGACAACCACTACCTGAGCACCCAGTCCGCCCTGAGCAA |
| | | AGACCCCAACGAGAAGCGCGATCACATGGTCCTGCTGGAGTTCGTG |
| | | ACCGCCGCCGGGATCACTCTCGGCATGGACGAGCTGTACAAGtaatagcg |
| | | gccgcgactctagatcataatcagccataccacatttgtagaggttttacttgctttaaaaaacctcccacacctcccc |
| | | ctgaacctgaaacataaaatgaatgcaattgttgttgttaacttgtttattgcagcttataatggttacaaataaagcaat |
| | | agcatcacaaatttcacaaataaagcatttttttcactgcattctagttgtggtttgtccaaactcatcaatgtatcttaag |
| | | gcgttagtctcctgatattgggtctaacccccacctcctgttaggcagattccttatctggtgacacacccccatttcct |
| | | ggagccatctctctccttgccagaacctctaaggtttgcttacgatggagccagagaggatcctgggagggagag |
| | | cttggcagggggtgggagggaagggggggatgcgtgacctgcccggttctcagtggccaccctgcgctaccct |
| | | ctcccagaacctgagctgctctgacgcggccgtctggtgcgtttcactgatcctggtgctgcagcttccttacacttc |
| | | ccaagaggagaagcagtttggaaaaacaaaatcagaataagttggtcctgagttctaactttggctcttcacctttcta |
| | | gtccccaatttatattgttcctccgtgcgtcagttttacctgtgagataaggccagtagccagccccgt |
| AAV6-231 | 721 | gaccactttgagctctactggcttctgcgccgcctctggcccactgtttccccttcccaggcaggtcctgctttctctg |
| GFP insert | | acctgcattctctcccctgggcctgtgccgctttctgtctgcagcttgtggcctgggtcacctctacggctggcccag |
| for AAVSI | | atccttccctgccgcctccttcaggttccgtcttcctccactccctcttccccttgctctctgctgtgttgctgcccaagg |
| | | atgctctttccggagcacttccttctcggcgctgcaccacgtgatgtcctctgagcggatcctccccgtgtctgggtc |
| | | ctctccgggcatctctcctccctcacccaaccccatgccgtcttcactcgctgggttcccttttccttctccttctgggg |
| | | cctgtgccatctctcgtttcttaggatggccttctccgacggatgtctcccttgcgtcccgcctccccttcttgtaggcc |
| | | tgcatcatcaccgtttttctggacaaccccaaagtaccccgtctccctggctttagccacctctccatcctcttgctttct |
| | | ttgcctggacaccccgttctcctgtggattcgggtcacctctcactcctttcatttgggcagctcccctaccccccttac |
| | | ctctctagtctgtgctagctcttccagccccctgtcatggcatcttccaggggtccgagagctcagctagtcttcttcct |
| | | ccaacccgggcccctatgtccacttcaggacagcatgtttgctgcctccagggatcctgtgtccccgagctgggac |
| | | caccttatattcccagggccggttaatgtggctctggttctgggtacttttatctgtcccctccaccccacagtggggc |
| | | cactagggacaggattggtgacagaaaagccccatccttaggcctcctccttagttattaatgagtaattcatacaaa |
| | | aggactcgcccctgccttggggaatcccagggaccgtcgttaaactcccactaacgtagaacccagagatcgctg |
| | | cgttcccgccccctcacccgcccgctctcgtcatcactgaggtggagaagagcatgcgtgaggctccggtgccc |
| | | gtcagtgggcagagcgcacatcgcccacagtccccgagaagttggggggaggggtcggcaattgaaccggtg |
| | | cctagagaaggtggcgcggggtaaactgggaaagtgatgtcgtgtactggctccgcctttttcccgagggtgggg |
| | | gagaaccgtatataagtgcagtagtcgccgtgaacgttctttttcgcaacgggtttgccgccagaacacaggtaagt |
| | | gccgtgtgtggttcccgcgggcctggcctctttacgggttatggcccttgcgtgccttgaattacttccacgcccctg |
| | | gctgcagtacgtgattcttgatcccgagcttcgggttggaagtgggtgggagagttcgaggccttgcgcttaagga |
| | | gccccttcgcctcgtgcttgagttgaggcctggcttgggcgctggggccgccgcgtgcgaatctggtggcaccttc |
| | | gcgcctgtctcgctgctttcgataagtctctagccatttaaaatttttgatgacctgctgcgacgctttttttctggcaag |
| | | atagtcttgtaaatgcgggccaaCatctgcacactggtatttcggtttttggggccgcgggcggcgacggggccc |
| | | gtgcgtcccagcgcacatgttcggcgaggcggggcctgcgagcgcggccaccgagaatcggacgggggtagt |
| | | ctcaagctggccggcctgctctggtgcctggcctcgcgccgccgtgtatcgccccgccctgggcggcaaggctg |
| | | gcccggtcggcaccagttgcgtgagcggaaagatggccgcttcccggccctgctgcagggagctcaaaatgga |
| | | ggacgcggcgctcgggagagcgggcgggtgagtcacccacacaaaggaaaagggcctttccgtcctcagccg |
| | | tcgcttcatgtgactccacggagtaccgggcgccgtccaggcacctcgattagttctcgagcttttggagtacgtcg |
| | | tctttaggttggggggaggggttttatgcgatggagtttccccacactgagtgggtggagactgaagttaggccag |
| | | cttggcacttgatgtaattctccttggaatttgccctttttgagtttggatcttggttcattctcaagcctcagacagtggtt |
| | | caaagtttttttcttccatttcaggtgtcgtgacgctagcgctaccggactcaatctcgagctcaagcttcgaattctgc |
| | | agtcgacggtaccgcgggcccgggatccaccggtcgccaccATGgtgAGCAAGGGCGAGGA |
| | | GCTGTTCACCGGGGTGGTGCCCATCCTGGTCGAGCTGGACGGCGACG |
| | | TAAACGGCCACAAGTTCAGCGTGTCCGGCGAGGGCGAGGGCGATGC |
| | | CACCTACGGCAAGCTGACCCTGAAGTTCATCTGCACCACCGGCAAGC |
| | | TGCCCGTGCCCTGGCCCACCCTCGTGACCACCCTGACCTACGGCGTG |
| | | CAGTGCTTCAGCCGCTACCCCGACCACATGAAGCAGCACGACTTCTT |
| | | CAAGTCCGCCATGCCCGAAGGCTACGTCCAGGAGCGCACCATCTTCT |
| | | TCAAGGACGACGGCAACTACAAGACCCGCGCCGAGGTGAAGTTCGA |
| | | GGGCGACACCCTGGTGAACCGCATCGAGCTGAAGGGCATCGACTTC |
| | | AAGGAGGACGGCAACATCCTGGGGCACAAGCTGGAGTACAACTACA |
| | | ACAGCCACAACGTCTATATCATGGCCGACAAGCAGAAGAACGGCAT |
| | | CAAGGTGAACTTCAAGATCCGCCACAACATCGAGGACGGCAGCGTG |
| | | CAGCTCGCCGACCACTACCAGCAGAACACCCCCATCGGCGACGGCC |
| | | CCGTGCTGCTGCCCGACAACCACTACCTGAGCACCCAGTCCGCCCTG |
| | | AGCAAAGACCCCAACGAGAAGCGCGATCACATGGTCCTGCTGGAGT |
| | | TCGTGACCGCCGCCGGGATCACTCTCGGCATGGACGAGCTGTACAAG |
| | | TAAtagcggccgcgactctagatcataatcagccataccacatttgtagaggttttacttgctttaaaaaacctccc |
| | | acacctccccctgaacctgaaacataaaatgaatgcaattgttgttgttaacttgtttattgcagcttataatggttacaa |
| | | ataaagcaatagcatcacaaatttcacaaataaagcatttttttcactgcattctagttgtggtttgtccaaactcatcaat |
| | | gtatcttaaggcgtgtctaacccccacctcctgttaggcagattccttatctggtgacacacccccatttcctggagcc |
| | | atctctctccttgccagaacctctaaggtttgcttacgatggagccagagaggatcctgggagggagagcttggca |
| | | gggggtgggagggaagggggggatgcgtgacctgcccggttctcagtggccaccctgcgctaccctctcccag |
| | | aacctgagctgctctgacgcggccgtctggtgcgtttcactgatcctggtgctgcagcttccttacacttcccaagag |
| | | gagaagcagtttggaaaaacaaaatcagaataagttggtcctgagttctaactttggctcttcacctttctagtcccca |
| | | atttatattgttcctccgtgcgtcagttttacctgtgagataaggccagtagccagccccgtcctggcagggctgtggt |
| | | gaggaggggggtgtccgtgtggaaaactccctttgtgagaatggtgcgtcctaggtgttcaccaggtcgtggccg |
| | | cctctactccctttctctttctccatccttctttccttaaagagtccccagtgctatctgggacatattcctccgcccagag |
| | | cagggtcccgcttccctaaggccctgctctgggcttctgggtttgagtccttggcaagcccaggagaggcgctcag |
| | | gcttccctgtcccccttcctcgtccaccatctcatgcccctggctctcctgccccttccctacaggggttcctggctct |
| | | gctcttcagactgagccccgttcccctgcatccccgttcccctgcatcccccttcccctgcatcccccagaggcccc |
| | | aggccacctacttggcctggaccccacgagaggccaccccagccctgtctaccaggctgccttttgggtggattct |
| | | cctccaa |
| AAV6-1018 | 722 | agatcttaatcttctgggtttccgttttctcgaatgaaaaatgcaggtccgagcagttaactggctggggcaccattag |
| GFP insert | | caagtcacttagcatctctggggccagtctgcaaagcgagggggcagccttaatgtgcctccagcctgaagtccta |
| for B2M | | gaatgagcgcccggtgtcccaagctggggcgcgcaccccagatcggagggcgccgatgtacagacagcaaac |
| | | tcacccagtctagtgcatgccttcttaaacatcacgagactctaagaaaaggaaactgaaaacgggaaagtccctct |
| | | ctctaacctggcactgcgtcgctggcttggagacaggtgacggtccctgcgggccttgtcctgattggctgggcac |
| | | gcgtttaatataagtggaggcgtcgcgctggcgggcattcctgaagctgacagcattcgggccgagaGGCTC |
| | | CGGTGCCCGTCAGTGGGCAGAGCGCACATCGCCCACAGTCCCCGAG |
| | | AAGTTGGGGGGAGGGGTCGGCAATTGAACCGGTGCCTAGAGAAGGT |
| | | GGCGCGGGGTAAACTGGGAAAGTGATGTCGTGTACTGGCTCCGCCTT |
| | | TTTCCCGAGGGTGGGGGAGAACCGTATATAAGTGCAGTAGTCGCCGT |
| | | GAACGTTCTTTTTCGCAACGGGTTTGCCGCCAGAACACAGGTAAGTG |
| | | CCGTGTGTGGTTCCCGCGGGCCTGGCCTCTTTACGGGTTATGGCCCTT |
| | | GCGTGCCTTGAATTACTTCCACGCCCCTGGCTGCAGTACGTGATTCTT |
| | | GATCCCGAGCTTCGGGTTGGAAGTGGGTGGGAGAGTTCGAGGCCTTG |
| | | CGCTTAAGGAGCCCCTTCGCCTCGTGCTTGAGTTGAGGCCTGGCTTG |
| | | GGCGCTGGGGCCGCCGCGTGCGAATCTGGTGGCACCTTCGCGCCTGT |
| | | CTCGCTGCTTTCGATAAGTCTCTAGCCATTTAAAATTTTTGATGACCT |
| | | GCTGCGACGCTTTTTTTCTGGCAAGATAGTCTTGTAAATGCGGGCCA |
| | | AGATgTGCACACTGGTATTTCGGTTTTTGGGGCCGCGGGCGGCGACG |
| | | GGGCCCGTGCGTCCCAGCGCACATGTTCGGCGAGGCGGGGCCTGCG |
| | | AGCGCGGCCACCGAGAATCGGACGGGGGTAGTCTCAAGCTGGCCGG |
| | | CCTGCTCTGGTGCCTGGCCTCGCGCCGCCGTGTATCGCCCCGCCCTG |
| | | GGCGGCAAGGCTGGCCCGGTCGGCACCAGTTGCGTGAGCGGAAAGA |
| | | TGGCCGCTTCCCGGCCCTGCTGCAGGGAGCTCAAAATGGAGGACGC |
| | | GGCGCTCGGGAGAGCGGGCGGGTGAGTCACCCACACAAAGGAAAAG |
| | | GGCCTTTCCGTCCTCAGCCGTCGCTTCATGTGACTCCACGGAGTACC |
| | | GGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCA |
| | | CACTGAGTGGGTGGAGACTGAAGTTAGGCCAGCTTGGCACTTGATGT |
| | | AATTCTCCTTGGAATTTGCCCTTTTTGAGTTTGGATCTTGGTTCATTCT |
| | | CAAGCCTCAGACAGTGGTTCAAAGTTTTTTTCTTCCATTTCAGGTGTC |
| | | GTGAcggCCGGCCCCGCCACCatggtgAGCAAGGGCGAGGAGCTGTTCAC |
| | | CGGGGTGGTGCCCATCCTGGTCGAGCTGGACGGCGACGTAAACGGC |
| | | CACAAGTTCAGCGTGTCCGGCGAGGGCGAGGGCGATGCCACCTACG |
| | | GCAAGCTGACCCTGAAGTTCATCTGCACCACCGGCAAGCTGCCCGTG |
| | | CCCTGGCCCACCCTCGTGACCACCCTGACCTACGGCGTGCAGTGCTT |
| | | CAGCCGCTACCCCGACCACATGAAGCAGCACGACTTCTTCAAGTCCG |
| | | CCATGCCCGAAGGCTACGTCCAGGAGCGCACCATCTTCTTCAAGGAC |
| | | GACGGCAACTACAAGACCCGCGCCGAGGTGAAGTTCGAGGGCGACA |
| | | CCCTGGTGAACCGCATCGAGCTGAAGGGCATCGACTTCAAGGAGGA |
| | | CGGCAACATCCTGGGGCACAAGCTGGAGTACAACTACAACAGCCAC |
| | | AACGTCTATATCATGGCCGACAAGCAGAAGAACGGCATCAAGGTGA |
| | | ACTTCAAGATCCGCCACAACATCGAGGACGGCAGCGTGCAGCTCGC |
| | | CGACCACTACCAGCAGAACACCCCCATCGGCGACGGCCCCGTGCTGC |
| | | TGCCCGACAACCACTACCTGAGCACCCAGTCCGCCCTGAGCAAAGAC |
| | | CCCAACGAGAAGCGCGATCACATGGTCCTGCTGGAGTTCGTGACCGC |
| | | CGCCGGGATCACTCTCGGCATGGACGAGCTGTACAAGTAATCTAGAc |
| | | ctCGACTGTGCCTTCTAGTTGCCAGCCATCTGTTGTTTGCCCCTCCCCC |
| | | GTGCCTTCCTTGACCCTGGAAGGTGCCACTCCCACTGTCCTTTCCTAA |
| | | TAAAATGAGGAAATTGCATCGCATTGTCTGAGTAGGTGTCATTCTAT |
| | | TCTGGGGGGTGGGGTGGGGCAGGACAGCAAGGGGGAGGATTGGGA |
| | | AGACAATAGCAGGCATGCTGGGGATGCGGTGGGCTCTATGGcttctgagg |
| | | cggaaagaaccagctggggctctagggggtatccccACTAGTtgtctcgctccgtggccttagctgtgctcg |
| | | cgctactctctctttctggcctggaggctatccagcgtgagtctctcctaccctcccgctctggtccttcctctcccgct |
| | | ctgcaccctctgtggccctcgctgtgctctctcgctccgtgacttcccttctccaagttctccttggtggcccgccgtg |
| | | gggctagtccagggctggatctcggggaagcggcggggtggcctgggagtggggaagggggtgcgcacccg |
| | | ggacgcgcgctacttgcccctttcggcggggagcaggggagacctttggcctacggcgacgggagggtcggg |
| | | acaaagtttagggcgtcgataagcgtcagagcgccgaggttgggggagggtttctcttccgctctttcgcggggcc |
| | | tctggctcccccagcgcagctggagtgggggacgggtaggct |
| G014832 | 723 | mG*mG*mC*UCUCGGAGAAUGACGAGGUUUUAGAmGmCmUmAmGm |
| | | AmAmAmUmAmGmCAAGUUAAAAUAAGGCUAGUCCGUUAUCAmAm |
| | | CmUmUmGmAmAmAmAmAmGmUmGmGmCmAmCmCmGmAmGmUmC |
| | | mGmGmUmGmCmU*mU*mU*mU |
| ORF | SEQ ID | ATGGACAAGAAGTACAGCATCGGACTGGACATCGGAACAAACAGCG |
| encoding | NO: 1 | TCGGATGGGCAGTCATCACAGACGAATACAAGGTCCCGAGCAAGAA |
| Sp. Cas9 | | GTTCAAGGTCCTGGGAAACACAGACAGACACAGCATCAAGAAGAAC |
| | | CTGATCGGAGCACTGCTGTTCGACAGCGGAGAAACAGCAGAAGCAA |
| | | CAAGACTGAAGAGAACAGCAAGAAGAAGATACACAAGAAGAAAGA |
| | | ACAGAATCTGCTACCTGCAGGAAATCTTCAGCAACGAAATGGCAAA |
| | | GGTCGACGACAGCTTCTTCCACAGACTGGAAGAAAGCTTCCTGGTCG |
| | | AAGAAGACAAGAAGCACGAAAGACACCCGATCTTCGGAAACATCGT |
| | | CGACGAAGTCGCATACCACGAAAAGTACCCGACAATCTACCACCTG |
| | | AGAAAGAAGCTGGTCGACAGCACAGACAAGGCAGACCTGAGACTGA |
| | | TCTACCTGGCACTGGCACACATGATCAAGTTCAGAGGACACTTCCTG |
| | | ATCGAAGGAGACCTGAACCCGGACAACAGCGACGTCGACAAGCTGT |
| | | TCATCCAGCTGGTCCAGACATACAACCAGCTGTTCGAAGAAAACCCG |
| | | ATCAACGCAAGCGGAGTCGACGCAAAGGCAATCCTGAGCGCAAGAC |
| | | TGAGCAAGAGCAGAAGACTGGAAAACCTGATCGCACAGCTGCCGGG |
| | | AGAAAAGAAGAACGGACTGTTCGGAAACCTGATCGCACTGAGCCTG |
| | | GGACTGACACCGAACTTCAAGAGCAACTTCGACCTGGCAGAAGACG |
| | | CAAAGCTGCAGCTGAGCAAGGACACATACGACGACGACCTGGACAA |
| | | CCTGCTGGCACAGATCGGAGACCAGTACGCAGACCTGTTCCTGGCAG |
| | | CAAAGAACCTGAGCGACGCAATCCTGCTGAGCGACATCCTGAGAGT |
| | | CAACACAGAAATCACAAAGGCACCGCTGAGCGCAAGCATGATCAAG |
| | | AGATACGACGAACACCACCAGGACCTGACACTGCTGAAGGCACTGG |
| | | TCAGACAGCAGCTGCCGGAAAAGTACAAGGAAATCTTCTTCGACCA |
| | | GAGCAAGAACGGATACGCAGGATACATCGACGGAGGAGCAAGCCA |
| | | GGAAGAATTCTACAAGTTCATCAAGCCGATCCTGGAAAAGATGGAC |
| | | GGAACAGAAGAACTGCTGGTCAAGCTGAACAGAGAAGACCTGCTGA |
| | | GAAAGCAGAGAACATTCGACAACGGAAGCATCCCGCACCAGATCCA |
| | | CCTGGGAGAACTGCACGCAATCCTGAGAAGACAGGAAGACTTCTAC |
| | | CCGTTCCTGAAGGACAACAGAGAAAAGATCGAAAAGATCCTGACAT |
| | | TCAGAATCCCGTACTACGTCGGACCGCTGGCAAGAGGAAACAGCAG |
| | | ATTCGCATGGATGACAAGAAAGAGCGAAGAAACAATCACACCGTGG |
| | | AACTTCGAAGAAGTCGTCGACAAGGGAGCAAGCGCACAGAGCTTCA |
| | | TCGAAAGAATGACAAACTTCGACAAGAACCTGCCGAACGAAAAGGT |
| | | CCTGCCGAAGCACAGCCTGCTGTACGAATACTTCACAGTCTACAACG |
| | | AACTGACAAAGGTCAAGTACGTCACAGAAGGAATGAGAAAGCCGGC |
| | | ATTCCTGAGCGGAGAACAGAAGAAGGCAATCGTCGACCTGCTGTTC |
| | | AAGACAAACAGAAAGGTCACAGTCAAGCAGCTGAAGGAAGACTACT |
| | | TCAAGAAGATCGAATGCTTCGACAGCGTCGAAATCAGCGGAGTCGA |
| | | AGACAGATTCAACGCAAGCCTGGGAACATACCACGACCTGCTGAAG |
| | | ATCATCAAGGACAAGGACTTCCTGGACAACGAAGAAAACGAAGACA |
| | | TCCTGGAAGACATCGTCCTGACACTGACACTGTTCGAAGACAGAGAA |
| | | ATGATCGAAGAAAGACTGAAGACATACGCACACCTGTTCGACGACA |
| | | AGGTCATGAAGCAGCTGAAGAGAAGAAGATACACAGGATGGGGAA |
| | | GACTGAGCAGAAAGCTGATCAACGGAATCAGAGACAAGCAGAGCGG |
| | | AAAGACAATCCTGGACTTCCTGAAGAGCGACGGATTCGCAAACAGA |
| | | AACTTCATGCAGCTGATCCACGACGACAGCCTGACATTCAAGGAAG |
| | | ACATCCAGAAGGCACAGGTCAGCGGACAGGGAGACAGCCTGCACGA |
| | | ACACATCGCAAACCTGGCAGGAAGCCCGGCAATCAAGAAGGGAATC |
| | | CTGCAGACAGTCAAGGTCGTCGACGAACTGGTCAAGGTCATGGGAA |
| | | GACACAAGCCGGAAAACATCGTCATCGAAATGGCAAGAGAAAACCA |
| | | GACAACACAGAAGGGACAGAAGAACAGCAGAGAAAGAATGAAGAG |
| | | AATCGAAGAAGGAATCAAGGAACTGGGAAGCCAGATCCTGAAGGAA |
| | | CACCCGGTCGAAAACACACAGCTGCAGAACGAAAAGCTGTACCTGT |
| | | ACTACCTGCAGAACGGAAGAGACATGTACGTCGACCAGGAACTGGA |
| | | CATCAACAGACTGAGCGACTACGACGTCGACCACATCGTCCCGCAG |
| | | AGCTTCCTGAAGGACGACAGCATCGACAACAAGGTCCTGACAAGAA |
| | | GCGACAAGAACAGAGGAAAGAGCGACAACGTCCCGAGCGAAGAAG |
| | | TCGTCAAGAAGATGAAGAACTACTGGAGACAGCTGCTGAACGCAAA |
| | | GCTGATCACACAGAGAAAGTTCGACAACCTGACAAAGGCAGAGAGA |
| | | GGAGGACTGAGCGAACTGGACAAGGCAGGATTCATCAAGAGACAGC |
| | | TGGTCGAAACAAGACAGATCACAAAGCACGTCGCACAGATCCTGGA |
| | | CAGCAGAATGAACACAAAGTACGACGAAAACGACAAGCTGATCAGA |
| | | GAAGTCAAGGTCATCACACTGAAGAGCAAGCTGGTCAGCGACTTCA |
| | | GAAAGGACTTCCAGTTCTACAAGGTCAGAGAAATCAACAACTACCA |
| | | CCACGCACACGACGCATACCTGAACGCAGTCGTCGGAACAGCACTG |
| | | ATCAAGAAGTACCCGAAGCTGGAAAGCGAATTCGTCTACGGAGACT |
| | | ACAAGGTCTACGACGTCAGAAAGATGATCGCAAAGAGCGAACAGGA |
| | | AATCGGAAAGGCAACAGCAAAGTACTTCTTCTACAGCAACATCATG |
| | | AACTTCTTCAAGACAGAAATCACACTGGCAAACGGAGAAATCAGAA |
| | | AGAGACCGCTGATCGAAACAAACGGAGAAACAGGAGAAATCGTCTG |
| | | GGACAAGGGAAGAGACTTCGCAACAGTCAGAAAGGTCCTGAGCATG |
| | | CCGCAGGTCAACATCGTCAAGAAGACAGAAGTCCAGACAGGAGGAT |
| | | TCAGCAAGGAAAGCATCCTGCCGAAGAGAAACAGCGACAAGCTGAT |
| | | CGCAAGAAAGAAGGACTGGGACCCGAAGAAGTACGGAGGATTCGAC |
| | | AGCCCGACAGTCGCATACAGCGTCCTGGTCGTCGCAAAGGTCGAAA |
| | | AGGGAAAGAGCAAGAAGCTGAAGAGCGTCAAGGAACTGCTGGGAA |
| | | TCACAATCATGGAAAGAAGCAGCTTCGAAAAGAACCCGATCGACTT |
| | | CCTGGAAGCAAAGGGATACAAGGAAGTCAAGAAGGACCTGATCATC |
| | | AAGCTGCCGAAGTACAGCCTGTTCGAACTGGAAAACGGAAGAAAGA |
| | | GAATGCTGGCAAGCGCAGGAGAACTGCAGAAGGGAAACGAACTGGC |
| | | ACTGCCGAGCAAGTACGTCAACTTCCTGTACCTGGCAAGCCACTACG |
| | | AAAAGCTGAAGGGAAGCCCGGAAGACAACGAACAGAAGCAGCTGTT |
| | | CGTCGAACAGCACAAGCACTACCTGGACGAAATCATCGAACAGATC |
| | | AGCGAATTCAGCAAGAGAGTCATCCTGGCAGACGCAAACCTGGACA |
| | | AGGTCCTGAGCGCATACAACAAGCACAGAGACAAGCCGATCAGAGA |
| | | ACAGGCAGAAAACATCATCCACCTGTTCACACTGACAAACCTGGGA |
| | | GCACCGGCAGCATTCAAGTACTTCGACACAACAATCGACAGAAAGA |
| | | GATACACAAGCACAAAGGAAGTCCTGGACGCAACACTGATCCACCA |
| | | GAGCATCACAGGACTGTACGAAACAAGAATCGACCTGAGCCAGCTG |
| | | GGAGGAGACGGAGGAGGAAGCCCGAAGAAGAAGAGAAAGGTCTAG |
| ORF | SEQ ID | ATGGACAAGAAGTACTCCATCGGCCTGGACATCGGCACCAACTCCGT |
| encoding | NO: 2 | GGGCTGGGCCGTGATCACCGACGAGTACAAGGTGCCCTCCAAGAAG |
| Sp. Cas9 | | TTCAAGGTGCTGGGCAACACCGACCGGCACTCCATCAAGAAGAACC |
| | | TGATCGGCGCCCTGCTGTTCGACTCCGGCGAGACCGCCGAGGCCACC |
| | | CGGCTGAAGCGGACCGCCCGGCGGCGGTACACCCGGCGGAAGAACC |
| | | GGATCTGCTACCTGCAGGAGATCTTCTCCAACGAGATGGCCAAGGTG |
| | | GACGACTCCTTCTTCCACCGGCTGGAGGAGTCCTTCCTGGTGGAGGA |
| | | GGACAAGAAGCACGAGCGGCACCCCATCTTCGGCAACATCGTGGAC |
| | | GAGGTGGCCTACCACGAGAAGTACCCCACCATCTACCACCTGCGGA |
| | | AGAAGCTGGTGGACTCCACCGACAAGGCCGACCTGCGGCTGATCTA |
| | | CCTGGCCCTGGCCCACATGATCAAGTTCCGGGGCCACTTCCTGATCG |
| | | AGGGCGACCTGAACCCCGACAACTCCGACGTGGACAAGCTGTTCATC |
| | | CAGCTGGTGCAGACCTACAACCAGCTGTTCGAGGAGAACCCCATCA |
| | | ACGCCTCCGGCGTGGACGCCAAGGCCATCCTGTCCGCCCGGCTGTCC |
| | | AAGTCCCGGCGGCTGGAGAACCTGATCGCCCAGCTGCCCGGCGAGA |
| | | AGAAGAACGGCCTGTTCGGCAACCTGATCGCCCTGTCCCTGGGCCTG |
| | | ACCCCCAACTTCAAGTCCAACTTCGACCTGGCCGAGGACGCCAAGCT |
| | | GCAGCTGTCCAAGGACACCTACGACGACGACCTGGACAACCTGCTG |
| | | GCCCAGATCGGCGACCAGTACGCCGACCTGTTCCTGGCCGCCAAGAA |
| | | CCTGTCCGACGCCATCCTGCTGTCCGACATCCTGCGGGTGAACACCG |
| | | AGATCACCAAGGCCCCCCTGTCCGCCTCCATGATCAAGCGGTACGAC |
| | | GAGCACCACCAGGACCTGACCCTGCTGAAGGCCCTGGTGCGGCAGC |
| | | AGCTGCCCGAGAAGTACAAGGAGATCTTCTTCGACCAGTCCAAGAA |
| | | CGGCTACGCCGGCTACATCGACGGCGGCGCCTCCCAGGAGGAGTTCT |
| | | ACAAGTTCATCAAGCCCATCCTGGAGAAGATGGACGGCACCGAGGA |
| | | GCTGCTGGTGAAGCTGAACCGGGAGGACCTGCTGCGGAAGCAGCGG |
| | | ACCTTCGACAACGGCTCCATCCCCCACCAGATCCACCTGGGCGAGCT |
| | | GCACGCCATCCTGCGGCGGCAGGAGGACTTCTACCCCTTCCTGAAGG |
| | | ACAACCGGGAGAAGATCGAGAAGATCCTGACCTTCCGGATCCCCTA |
| | | CTACGTGGGCCCCCTGGCCCGGGGCAACTCCCGGTTCGCCTGGATGA |
| | | CCCGGAAGTCCGAGGAGACCATCACCCCCTGGAACTTCGAGGAGGT |
| | | GGTGGACAAGGGCGCCTCCGCCCAGTCCTTCATCGAGCGGATGACCA |
| | | ACTTCGACAAGAACCTGCCCAACGAGAAGGTGCTGCCCAAGCACTC |
| | | CCTGCTGTACGAGTACTTCACCGTGTACAACGAGCTGACCAAGGTGA |
| | | AGTACGTGACCGAGGGCATGCGGAAGCCCGCCTTCCTGTCCGGCGA |
| | | GCAGAAGAAGGCCATCGTGGACCTGCTGTTCAAGACCAACCGGAAG |
| | | GTGACCGTGAAGCAGCTGAAGGAGGACTACTTCAAGAAGATCGAGT |
| | | GCTTCGACTCCGTGGAGATCTCCGGCGTGGAGGACCGGTTCAACGCC |
| | | TCCCTGGGCACCTACCACGACCTGCTGAAGATCATCAAGGACAAGG |
| | | ACTTCCTGGACAACGAGGAGAACGAGGACATCCTGGAGGACATCGT |
| | | GCTGACCCTGACCCTGTTCGAGGACCGGGAGATGATCGAGGAGCGG |
| | | CTGAAGACCTACGCCCACCTGTTCGACGACAAGGTGATGAAGCAGCT |
| | | GAAGCGGCGGCGGTACACCGGCTGGGGCCGGCTGTCCCGGAAGCTG |
| | | ATCAACGGCATCCGGGACAAGCAGTCCGGCAAGACCATCCTGGACT |
| | | TCCTGAAGTCCGACGGCTTCGCCAACCGGAACTTCATGCAGCTGATC |
| | | CACGACGACTCCCTGACCTTCAAGGAGGACATCCAGAAGGCCCAGG |
| | | TGTCCGGCCAGGGCGACTCCCTGCACGAGCACATCGCCAACCTGGCC |
| | | GGCTCCCCCGCCATCAAGAAGGGCATCCTGCAGACCGTGAAGGTGG |
| | | TGGACGAGCTGGTGAAGGTGATGGGCCGGCACAAGCCCGAGAACAT |
| | | CGTGATCGAGATGGCCCGGGAGAACCAGACCACCCAGAAGGGCCAG |
| | | AAGAACTCCCGGGAGCGGATGAAGCGGATCGAGGAGGGCATCAAGG |
| | | AGCTGGGCTCCCAGATCCTGAAGGAGCACCCCGTGGAGAACACCCA |
| | | GCTGCAGAACGAGAAGCTGTACCTGTACTACCTGCAGAACGGCCGG |
| | | GACATGTACGTGGACCAGGAGCTGGACATCAACCGGCTGTCCGACT |
| | | ACGACGTGGACCACATCGTGCCCCAGTCCTTCCTGAAGGACGACTCC |
| | | ATCGACAACAAGGTGCTGACCCGGTCCGACAAGAACCGGGGCAAGT |
| | | CCGACAACGTGCCCTCCGAGGAGGTGGTGAAGAAGATGAAGAACTA |
| | | CTGGCGGCAGCTGCTGAACGCCAAGCTGATCACCCAGCGGAAGTTC |
| | | GACAACCTGACCAAGGCCGAGCGGGGCGGCCTGTCCGAGCTGGACA |
| | | AGGCCGGCTTCATCAAGCGGCAGCTGGTGGAGACCCGGCAGATCAC |
| | | CAAGCACGTGGCCCAGATCCTGGACTCCCGGATGAACACCAAGTAC |
| | | GACGAGAACGACAAGCTGATCCGGGAGGTGAAGGTGATCACCCTGA |
| | | AGTCCAAGCTGGTGTCCGACTTCCGGAAGGACTTCCAGTTCTACAAG |
| | | GTGCGGGAGATCAACAACTACCACCACGCCCACGACGCCTACCTGA |
| | | ACGCCGTGGTGGGCACCGCCCTGATCAAGAAGTACCCCAAGCTGGA |
| | | GTCCGAGTTCGTGTACGGCGACTACAAGGTGTACGACGTGCGGAAG |
| | | ATGATCGCCAAGTCCGAGCAGGAGATCGGCAAGGCCACCGCCAAGT |
| | | ACTTCTTCTACTCCAACATCATGAACTTCTTCAAGACCGAGATCACCC |
| | | TGGCCAACGGCGAGATCCGGAAGCGGCCCCTGATCGAGACCAACGG |
| | | CGAGACCGGCGAGATCGTGTGGGACAAGGGCCGGGACTTCGCCACC |
| | | GTGCGGAAGGTGCTGTCCATGCCCCAGGTGAACATCGTGAAGAAGA |
| | | CCGAGGTGCAGACCGGCGGCTTCTCCAAGGAGTCCATCCTGCCCAAG |
| | | CGGAACTCCGACAAGCTGATCGCCCGGAAGAAGGACTGGGACCCCA |
| | | AGAAGTACGGCGGCTTCGACTCCCCCACCGTGGCCTACTCCGTGCTG |
| | | GTGGTGGCCAAGGTGGAGAAGGGCAAGTCCAAGAAGCTGAAGTCCG |
| | | TGAAGGAGCTGCTGGGCATCACCATCATGGAGCGGTCCTCCTTCGAG |
| | | AAGAACCCCATCGACTTCCTGGAGGCCAAGGGCTACAAGGAGGTGA |
| | | AGAAGGACCTGATCATCAAGCTGCCCAAGTACTCCCTGTTCGAGCTG |
| | | GAGAACGGCCGGAAGCGGATGCTGGCCTCCGCCGGCGAGCTGCAGA |
| | | AGGGCAACGAGCTGGCCCTGCCCTCCAAGTACGTGAACTTCCTGTAC |
| | | CTGGCCTCCCACTACGAGAAGCTGAAGGGCTCCCCCGAGGACAACG |
| | | AGCAGAAGCAGCTGTTCGTGGAGCAGCACAAGCACTACCTGGACGA |
| | | GATCATCGAGCAGATCTCCGAGTTCTCCAAGCGGGTGATCCTGGCCG |
| | | ACGCCAACCTGGACAAGGTGCTGTCCGCCTACAACAAGCACCGGGA |
| | | CAAGCCCATCCGGGAGCAGGCCGAGAACATCATCCACCTGTTCACCC |
| | | TGACCAACCTGGGCGCCCCCGCCGCCTTCAAGTACTTCGACACCACC |
| | | ATCGACCGGAAGCGGTACACCTCCACCAAGGAGGTGCTGGACGCCA |
| | | CCCTGATCCACCAGTCCATCACCGGCCTGTACGAGACCCGGATCGAC |
| | | CTGTCCCAGCTGGGCGGCGACGGCGGCGGCTCCCCCAAGAAGAAGC |
| | | GGAAGGTGTGA |
| open reading | SEQ ID | AUGGACAAGAAGUACUCCAUCGGCCUGGACAUCGGCACCAACUCC |
| frame for | NO: 3 | GUGGGCUGGGCCGUGAUCACCGACGAGUACAAGGUGCCCUCCAAG |
| Cas9 with | | AAGUUCAAGGUGCUGGGCAACACCGACCGGCACUCCAUCAAGAAG |
| Hibit tag | | AACCUGAUCGGCGCCCUGCUGUUCGACUCCGGCGAGACCGCCGAGG |
| | | CCACCCGGCUGAAGCGGACCGCCCGGCGGCGGUACACCCGGCGGAA |
| | | GAACCGGAUCUGCUACCUGCAGGAGAUCUUCUCCAACGAGAUGGC |
| | | CAAGGUGGACGACUCCUUCUUCCACCGGCUGGAGGAGUCCUUCCU |
| | | GGUGGAGGAGGACAAGAAGCACGAGCGGCACCCCAUCUUCGGCAA |
| | | CAUCGUGGACGAGGUGGCCUACCACGAGAAGUACCCCACCAUCUA |
| | | CCACCUGCGGAAGAAGCUGGUGGACUCCACCGACAAGGCCGACCU |
| | | GCGGCUGAUCUACCUGGCCCUGGCCCACAUGAUCAAGUUCCGGGG |
| | | CCACUUCCUGAUCGAGGGCGACCUGAACCCCGACAACUCCGACGUG |
| | | GACAAGCUGUUCAUCCAGCUGGUGCAGACCUACAACCAGCUGUUC |
| | | GAGGAGAACCCCAUCAACGCCUCCGGCGUGGACGCCAAGGCCAUCC |
| | | UGUCCGCCCGGCUGUCCAAGUCCCGGCGGCUGGAGAACCUGAUCGC |
| | | CCAGCUGCCCGGCGAGAAGAAGAACGGCCUGUUCGGCAACCUGAU |
| | | CGCCCUGUCCCUGGGCCUGACCCCCAACUUCAAGUCCAACUUCGAC |
| | | CUGGCCGAGGACGCCAAGCUGCAGCUGUCCAAGGACACCUACGAC |
| | | GACGACCUGGACAACCUGCUGGCCCAGAUCGGCGACCAGUACGCCG |
| | | ACCUGUUCCUGGCCGCCAAGAACCUGUCCGACGCCAUCCUGCUGUC |
| | | CGACAUCCUGCGGGUGAACACCGAGAUCACCAAGGCCCCCCUGUCC |
| | | GCCUCCAUGAUCAAGCGGUACGACGAGCACCACCAGGACCUGACCC |
| | | UGCUGAAGGCCCUGGUGCGGCAGCAGCUGCCCGAGAAGUACAAGG |
| | | AGAUCUUCUUCGACCAGUCCAAGAACGGCUACGCCGGCUACAUCG |
| | | ACGGCGGCGCCUCCCAGGAGGAGUUCUACAAGUUCAUCAAGCCCA |
| | | UCCUGGAGAAGAUGGACGGCACCGAGGAGCUGCUGGUGAAGCUGA |
| | | ACCGGGAGGACCUGCUGCGGAAGCAGCGGACCUUCGACAACGGCU |
| | | CCAUCCCCCACCAGAUCCACCUGGGCGAGCUGCACGCCAUCCUGCG |
| | | GCGGCAGGAGGACUUCUACCCCUUCCUGAAGGACAACCGGGAGAA |
| | | GAUCGAGAAGAUCCUGACCUUCCGGAUCCCCUACUACGUGGGCCCC |
| | | CUGGCCCGGGGCAACUCCCGGUUCGCCUGGAUGACCCGGAAGUCCG |
| | | AGGAGACCAUCACCCCCUGGAACUUCGAGGAGGUGGUGGACAAGG |
| | | GCGCCUCCGCCCAGUCCUUCAUCGAGCGGAUGACCAACUUCGACAA |
| | | GAACCUGCCCAACGAGAAGGUGCUGCCCAAGCACUCCCUGCUGUAC |
| | | GAGUACUUCACCGUGUACAACGAGCUGACCAAGGUGAAGUACGUG |
| | | ACCGAGGGCAUGCGGAAGCCCGCCUUCCUGUCCGGCGAGCAGAAG |
| | | AAGGCCAUCGUGGACCUGCUGUUCAAGACCAACCGGAAGGUGACC |
| | | GUGAAGCAGCUGAAGGAGGACUACUUCAAGAAGAUCGAGUGCUUC |
| | | GACUCCGUGGAGAUCUCCGGCGUGGAGGACCGGUUCAACGCCUCC |
| | | CUGGGCACCUACCACGACCUGCUGAAGAUCAUCAAGGACAAGGAC |
| | | UUCCUGGACAACGAGGAGAACGAGGACAUCCUGGAGGACAUCGUG |
| | | CUGACCCUGACCCUGUUCGAGGACCGGGAGAUGAUCGAGGAGCGG |
| | | CUGAAGACCUACGCCCACCUGUUCGACGACAAGGUGAUGAAGCAG |
| | | CUGAAGCGGCGGCGGUACACCGGCUGGGGCCGGCUGUCCCGGAAG |
| | | CUGAUCAACGGCAUCCGGGACAAGCAGUCCGGCAAGACCAUCCUG |
| | | GACUUCCUGAAGUCCGACGGCUUCGCCAACCGGAACUUCAUGCAG |
| | | CUGAUCCACGACGACUCCCUGACCUUCAAGGAGGACAUCCAGAAG |
| | | GCCCAGGUGUCCGGCCAGGGCGACUCCCUGCACGAGCACAUCGCCA |
| | | ACCUGGCCGGCUCCCCCGCCAUCAAGAAGGGCAUCCUGCAGACCGU |
| | | GAAGGUGGUGGACGAGCUGGUGAAGGUGAUGGGCCGGCACAAGCC |
| | | CGAGAACAUCGUGAUCGAGAUGGCCCGGGAGAACCAGACCACCCA |
| | | GAAGGGCCAGAAGAACUCCCGGGAGCGGAUGAAGCGGAUCGAGGA |
| | | GGGCAUCAAGGAGCUGGGCUCCCAGAUCCUGAAGGAGCACCCCGU |
| | | GGAGAACACCCAGCUGCAGAACGAGAAGCUGUACCUGUACUACCU |
| | | GCAGAACGGCCGGGACAUGUACGUGGACCAGGAGCUGGACAUCAA |
| | | CCGGCUGUCCGACUACGACGUGGACCACAUCGUGCCCCAGUCCUUC |
| | | CUGAAGGACGACUCCAUCGACAACAAGGUGCUGACCCGGUCCGAC |
| | | AAGAACCGGGGCAAGUCCGACAACGUGCCCUCCGAGGAGGUGGUG |
| | | AAGAAGAUGAAGAACUACUGGCGGCAGCUGCUGAACGCCAAGCUG |
| | | AUCACCCAGCGGAAGUUCGACAACCUGACCAAGGCCGAGCGGGGC |
| | | GGCCUGUCCGAGCUGGACAAGGCCGGCUUCAUCAAGCGGCAGCUG |
| | | GUGGAGACCCGGCAGAUCACCAAGCACGUGGCCCAGAUCCUGGAC |
| | | UCCCGGAUGAACACCAAGUACGACGAGAACGACAAGCUGAUCCGG |
| | | GAGGUGAAGGUGAUCACCCUGAAGUCCAAGCUGGUGUCCGACUUC |
| | | CGGAAGGACUUCCAGUUCUACAAGGUGCGGGAGAUCAACAACUAC |
| | | CACCACGCCCACGACGCCUACCUGAACGCCGUGGUGGGCACCGCCC |
| | | UGAUCAAGAAGUACCCCAAGCUGGAGUCCGAGUUCGUGUACGGCG |
| | | ACUACAAGGUGUACGACGUGCGGAAGAUGAUCGCCAAGUCCGAGC |
| | | AGGAGAUCGGCAAGGCCACCGCCAAGUACUUCUUCUACUCCAACA |
| | | UCAUGAACUUCUUCAAGACCGAGAUCACCCUGGCCAACGGCGAGA |
| | | UCCGGAAGCGGCCCCUGAUCGAGACCAACGGCGAGACCGGCGAGA |
| | | UCGUGUGGGACAAGGGCCGGGACUUCGCCACCGUGCGGAAGGUGC |
| | | UGUCCAUGCCCCAGGUGAACAUCGUGAAGAAGACCGAGGUGCAGA |
| | | CCGGCGGCUUCUCCAAGGAGUCCAUCCUGCCCAAGCGGAACUCCGA |
| | | CAAGCUGAUCGCCCGGAAGAAGGACUGGGACCCCAAGAAGUACGG |
| | | CGGCUUCGACUCCCCCACCGUGGCCUACUCCGUGCUGGUGGUGGCC |
| | | AAGGUGGAGAAGGGCAAGUCCAAGAAGCUGAAGUCCGUGAAGGAG |
| | | CUGCUGGGCAUCACCAUCAUGGAGCGGUCCUCCUUCGAGAAGAAC |
| | | CCCAUCGACUUCCUGGAGGCCAAGGGCUACAAGGAGGUGAAGAAG |
| | | GACCUGAUCAUCAAGCUGCCCAAGUACUCCCUGUUCGAGCUGGAG |
| | | AACGGCCGGAAGCGGAUGCUGGCCUCCGCCGGCGAGCUGCAGAAG |
| | | GGCAACGAGCUGGCCCUGCCCUCCAAGUACGUGAACUUCCUGUACC |
| | | UGGCCUCCCACUACGAGAAGCUGAAGGGCUCCCCCGAGGACAACG |
| | | AGCAGAAGCAGCUGUUCGUGGAGCAGCACAAGCACUACCUGGACG |
| | | AGAUCAUCGAGCAGAUCUCCGAGUUCUCCAAGCGGGUGAUCCUGG |
| | | CCGACGCCAACCUGGACAAGGUGCUGUCCGCCUACAACAAGCACCG |
| | | GGACAAGCCCAUCCGGGAGCAGGCCGAGAACAUCAUCCACCUGUU |
| | | CACCCUGACCAACCUGGGCGCCCCCGCCGCCUUCAAGUACUUCGAC |
| | | ACCACCAUCGACCGGAAGCGGUACACCUCCACCAAGGAGGUGCUG |
| | | GACGCCACCCUGAUCCACCAGUCCAUCACCGGCCUGUACGAGACCC |
| | | GGAUCGACCUGUCCCAGCUGGGCGGCGACGGCGGCGGCUCCCCCAA |
| | | GAAGAAGCGGAAGGUGUCCGAGUCCGCCACCCCCGAGUCCGUGUC |
| | | CGGCUGGCGGCUGUUCAAGAAGAUCUCCUGA |
| Not used | SEQ ID | |
| | NO: 4 | |
| Not used | SEQ ID | |
| | NO: 5 | |
| amino acid | SEQ ID | MDKKYSIGLDIGTNSVGWAVITDEYKVPSKKFKVLGNTDRHSIKKNLIG |
| sequence | NO: 6 | ALLFDSGETAEATRLKRTARRRYTRRKNRICYLQEIFSNEMAKVDDSFF |
| encoded by | | HRLEESFLVEEDKKHERHPIFGNIVDEVAYHEKYPTIYHLRKKLVDSTD |
| SEQ ID | | KADLRLIYLALAHMIKFRGHFLIEGDLNPDNSDVDKLFIQLVQTYNQLFE |
| NOs: 1-3 of | | ENPINASGVDAKAILSARLSKSRRLENLIAQLPGEKKNGLFGNLIALSLG |
| Cas9 | | LTPNFKSNFDLAEDAKLQLSKDTYDDDLDNLLAQIGDQYADLFLAAKN |
| | | LSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNR |
| | | EDLLRKQRTFDNGSIPHQIHLGELHAILRRQEDFYPFLKDNREKIEKILTF |
| | | RIPYYVGPLARGNSRFAWMTRKSEETITPWNFEEVVDKGASAQSFIERM |
| | | TNFDKNLPNEKVLPKHSLLYEYFTVYNELTKVKYVTEGMRKPAFLSGE |
| | | QKKAIVDLLFKTNRKVTVKQLKEDYFKKIECFDSVEISGVEDRFNASLG |
| | | TYHDLLKIIKDKDFLDNEENEDILEDIVLTLTLFEDREMIEERLKTYAHLF |
| | | DDKVMKQLKRRRYTGWGRLSRKLINGIRDKQSGKTILDFLKSDGFANR |
| | | NFMQLIHDDSLTFKEDIQKAQVSGQGDSLHEHIANLAGSPAIKKGILQTV |
| | | KVVDELVKVMGRHKPENIVIEMARENQTTQKGQKNSRERMKRIEEGIK |
| | | ELGSQILKEHPVENTQLQNEKLYLYYLQNGRDMYVDQELDINRLSDYD |
| | | VDHIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVAQIL |
| | | DSRMNTKYDENDKLIREVKVITLKSKLVSDFRKDFQFYKVREINNYHHA |
| | | HDAYLNAVVGTALIKKYPKLESEFVYGDYKVYDVRKMIAKSEQEIGKA |
| | | TAKYFFYSNIMNFFKTEITLANGEIRKRPLIETNGETGEIVWDKGRDFAT |
| | | VRKVLSMPQVNIVKKTEVQTGGFSKESILPKRNSDKLIARKKDWDPKK |
| | | YGGFDSPTVAYSVLVVAKVEKGKSKKLKSVKELLGITIMERSSFEKNPID |
| | | FLEAKGYKEVKKDLIIKLPKYSLFELENGRKRMLASAGELQKGNELALP |
| | | SKYVNFLYLASHYEKLKGSPEDNEQKQLFVEQHKHYLDEIIEQISEFSKR |
| | | VILADANLDKVLSAYNKHRDKPIREQAENIIHLFTLTNLGAPAAFKYFDT |
| | | TIDRKRYTSTKEVLDATLIHQSITGLYETRIDLSQLGGDGGGSPKKKRKV |
| | | * |
| amino acid | SEQ ID | MDKKYSIGLDIGTNSVGWAVITDEYKVPSKKFKVLGNTDRHSIKKNLIG |
| sequence for | NO: 7 | ALLFDSGETAEATRLKRTARRRYTRRKNRICYLQEIFSNEMAKVDDSFF |
| Sp Cas9- | | HRLEESFLVEEDKKHERHPIFGNIVDEVAYHEKYPTIYHLRKKLVDSTD |
| Hibit fusion | | KADLRLIYLALAHMIKFRGHFLIEGDLNPDNSDVDKLFIQLVQTYNQLFE |
| | | ENPINASGVDAKAILSARLSKSRRLENLIAQLPGEKKNGLFGNLIALSLG |
| | | LTPNFKSNFDLAEDAKLQLSKDTYDDDLDNLLAQIGDQYADLFLAAKN |
| | | LSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNR |
| | | EDLLRKQRTFDNGSIPHQIHLGELHAILRRQEDFYPFLKDNREKIEKILTF |
| | | RIPYYVGPLARGNSRFAWMTRKSEETITPWNFEEVVDKGASAQSFIERM |
| | | TNFDKNLPNEKVLPKHSLLYEYFTVYNELTKVKYVTEGMRKPAFLSGE |
| | | QKKAIVDLLFKTNRKVTVKQLKEDYFKKIECFDSVEISGVEDRFNASLG |
| | | TYHDLLKIIKDKDFLDNEENEDILEDIVLTLTLFEDREMIEERLKTYAHLF |
| | | DDKVMKQLKRRRYTGWGRLSRKLINGIRDKQSGKTILDFLKSDGFANR |
| | | NFMQLIHDDSLTFKEDIQKAQVSGQGDSLHEHIANLAGSPAIKKGILQTV |
| | | KVVDELVKVMGRHKPENIVIEMARENQTTQKGQKNSRERMKRIEEGIK |
| | | ELGSQILKEHPVENTQLQNEKLYLYYLQNGRDMYVDQELDINRLSDYD |
| | | VDHIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVAQIL |
| | | DSRMNTKYDENDKLIREVKVITLKSKLVSDFRKDFQFYKVREINNYHHA |
| | | HDAYLNAVVGTALIKKYPKLESEFVYGDYKVYDVRKMIAKSEQEIGKA |
| | | TAKYFFYSNIMNFFKTEITLANGEIRKRPLIETNGETGEIVWDKGRDFAT |
| | | VRKVLSMPQVNIVKKTEVQTGGFSKESILPKRNSDKLIARKKDWDPKK |
| | | YGGFDSPTVAYSVLVVAKVEKGKSKKLKSVKELLGITIMERSSFEKNPID |
| | | FLEAKGYKEVKKDLIIKLPKYSLFELENGRKRMLASAGELQKGNELALP |
| | | SKYVNFLYLASHYEKLKGSPEDNEQKQLFVEQHKHYLDEIIEQISEFSKR |
| | | VILADANLDKVLSAYNKHRDKPIREQAENIIHLFTLTNLGAPAAFKYFDT |
| | | TIDRKRYTSTKEVLDATLIHQSITGLYETRIDLSQLGGDGGGSPKKKRKV |
| | | SESATPESVSGWRLFKKIS |
| GFP insert | SEQ ID | ATGGTGAGCAAGGGCGAGGAGCTGTTCACCGGGGTGGTGCCCATCC |
| forHDRT- | NO: 8 | TGGTCGAGCTGGACGGCGACGTAAACGGCCACAAGTTCAGCGTGTC |
| GFP: | | CGGCGAGGGCGAGGGCGATGCCACCTACGGCAAGCTGACCCTGAAG |
| P00894 | | TTCATCTGCACCACCGGCAAGCTGCCCGTGCCCTGGCCCACCCTCGT |
| | | GACCACCCTGACCTACGGCGTGCAGTGCTTCAGCCGCTACCCCGACC |
| | | ACATGAAGCAGCACGACTTCTTCAAGTCCGCCATGCCCGAAGGCTAC |
| | | GTCCAGGAGCGCACCATCTTCTTCAAGGACGACGGCAACTACAAGA |
| | | CCCGCGCCGAGGTGAAGTTCGAGGGCGACACCCTGGTGAACCGCAT |
| | | CGAGCTGAAGGGCATCGACTTCAAGGAGGACGGCAACATCCTGGGG |
| | | CACAAGCTGGAGTACAACTACAACAGCCACAACGTCTATATCATGGC |
| | | CGACAAGCAGAAGAACGGCATCAAGGTGAACTTCAAGATCCGCCAC |
| | | AACATCGAGGACGGCAGCGTGCAGCTCGCCGACCACTACCAGCAGA |
| | | ACACCCCCATCGGCGACGGCCCCGTGCTGCTGCCCGACAACCACTAC |
| | | CTGAGCACCCAGTCCGCCCTGAGCAAAGACCCCAACGAGAAGCGCG |
| | | ATCACATGGTCCTGCTGGAGTTCGTGACCGCCGCCGGGATCACTCTC |
| | | GGCATGGACGAGCTGTACAAGTAA |
| Full HDRT | SEQ ID | tgccaacataccataaacctcccattctgctaatgcccagcctaagttggggagaccactccagattccaagatgta |
| template- | NO: 9 | cagtttgctttgctgggcctttttcccatgcctgcctttactctgccagagttatattgctggggttttgaagaagatccta |
| transgenic | | ttaaataaaagaataagcagtattattaagtagccctgcatttcaggtttccttgagtggcaggccaggcctggccgt |
| WTI TCR | | gaacgttcactgaaatcatggcctcttggccaagattgatagcttgtgcctgtccctgagtcccagtccatcacgagc |
| and TRAC | | agctggtttctaagatgctatttcccgtataaagcatgagaccgtgacttgccagccccacagagccccgcccttgt |
| homology | | ccatcactggcatctggactccagcctgggttggggcaaagagggaaatgagatcatgtcctaaccctgatcctctt |
| arms | | gtcccacagATATCCAGAACCCTGACCCTGCGGCTCCGGTGCCCGTCAGT |
| | | GGGCAGAGCGCACATCGCCCACAGTCCCCGAGAAGTTGGGGGGAGG |
| | | GGTCGGCAATTGAACCGGTGCCTAGAGAAGGTGGCGCGGGGTAAAC |
| | | TGGGAAAGTGATGTCGTGTACTGGCTCCGCCTTTTTCCCGAGGGTGG |
| | | GGGAGAACCGTATATAAGTGCAGTAGTCGCCGTGAACGTTCTTTTTC |
| | | GCAACGGGTTTGCCGCCAGAACACAGGTAAGTGCCGTGTGTGGTTCC |
| | | CGCGGGCCTGGCCTCTTTACGGGTTATGGCCCTTGCGTGCCTTGAATT |
| | | ACTTCCACGCCCCTGGCTGCAGTACGTGATTCTTGATCCCGAGCTTCG |
| | | GGTTGGAAGTGGGTGGGAGAGTTCGAGGCCTTGCGCTTAAGGAGCC |
| | | CCTTCGCCTCGTGCTTGAGTTGAGGCCTGGCTTGGGCGCTGGGGCCG |
| | | CCGCGTGCGAATCTGGTGGCACCTTCGCGCCTGTCTCGCTGCTTTCGA |
| | | TAAGTCTCTAGCCATTTAAAATTTTTGATGACCTGCTGCGACGCTTTT |
| | | TTTCTGGCAAGATAGTCTTGTAAATGCGGGCCAAGATgTGCACACTG |
| | | GTATTTCGGTTTTTGGGGCCGCGGGCGGCGACGGGGCCCGTGCGTCC |
| | | CAGCGCACATGTTCGGCGAGGCGGGGCCTGCGAGCGCGGCCACCGA |
| | | GAATCGGACGGGGGTAGTCTCAAGCTGGCCGGCCTGCTCTGGTGCCT |
| | | GGCCTCGCGCCGCCGTGTATCGCCCCGCCCTGGGCGGCAAGGCTGGC |
| | | CCGGTCGGCACCAGTTGCGTGAGCGGAAAGATGGCCGCTTCCCGGCC |
| | | CTGCTGCAGGGAGCTCAAAATGGAGGACGCGGCGCTCGGGAGAGCG |
| | | GGCGGGTGAGTCACCCACACAAAGGAAAAGGGCCTTTCCGTCCTCA |
| | | GCCGTCGCTTCATGTGACTCCACGGAGTACCGGGCGCCGTCCAGGCA |
| | | CCTCGATTAGTTCTCGAGCTTTTGGAGTACGTCGTCTTTAGGTTGGGG |
| | | GGAGGGGTTTTATGCGATGGAGTTTCCCCACACTGAGTGGGTGGAGA |
| | | CTGAAGTTAGGCCAGCTTGGCACTTGATGTAATTCTCCTTGGAATTTG |
| | | CCCTTTTTGAGTTTGGATCTTGGTTCATTCTCAAGCCTCAGACAGTGG |
| | | TTCAAAGTTTTTTTCTTCCATTTCAGGTGTCGTGAtGCGGCCGCCACC |
| | | ATGGGATCTTGGACACTGTGTTGCGTGTCCCTGTGCATCCTGGTGGC |
| | | CAAGCACACAGATGCCGGCGTGATCCAGTCTCCTAGACACGAAGTG |
| | | ACCGAGATGGGCCAAGAAGTGACCCTGCGCTGCAAGCCTATCAGCG |
| | | GCCACGATTACCTGTTCTGGTACAGACAGACCATGATGAGAGGCCTG |
| | | GAACTGCTGATCTACTTCAACAACAACGTGCCCATCGACGACAGCGG |
| | | CATGCCCGAGGATAGATTCAGCGCCAAGATGCCCAACGCCAGCTTCA |
| | | GCACCCTGAAGATCCAGCCTAGCGAGCCCAGAGATAGCGCCGTGTA |
| | | CTTCTGCGCCAGCAGAAAGACAGGCGGCTACAGCAATCAGCCCCAG |
| | | CACTTTGGAGATGGCACCCGGCTGAGCATCCTGGAAGATCTGAAGA |
| | | ACGTGTTCCCACCTGAGGTGGCCGTGTTCGAGCCTTCTGAGGCCGAG |
| | | ATCAGCCACACACAGAAAGCCACACTCGTGTGTCTGGCCACCGGCTT |
| | | CTATCCCGATCACGTGGAACTGTCTTGGTGGGTCAACGGCAAAGAGG |
| | | TGCACAGCGGCGTCAGCACCGATCCTCAGCCTCTGAAAGAGCAGCCC |
| | | GCTCTGAACGACAGCAGATACTGCCTGAGCAGCAGACTGAGAGTGT |
| | | CCGCCACCTTCTGGCAGAACCCCAGAAACCACTTCAGATGCCAGGTG |
| | | CAGTTCTACGGCCTGAGCGAGAACGATGAGTGGACCCAGGATAGAG |
| | | CCAAGCCTGTGACACAGATCGTGTCTGCCGAAGCCTGGGGCAGAGC |
| | | CGATTGTGGCTTTACCAGCGAGAGCTACCAGCAGGGCGTGCTGTCTG |
| | | CCACAATCCTGTACGAGATCCTGCTGGGCAAAGCCACTCTGTACGCC |
| | | GTGCTGGTGTCTGCCCTGGTGCTGATGGCCATGGTCAAGCGGAAGGA |
| | | TAGCAGGGGCGGCTCCGGTGCCACAAACTTCTCCCTGCTCAAGCAGG |
| | | CCGGAGATGTGGAAGAGAACCCTGGCCCTATGGAAACCCTGCTGAA |
| | | GGTGCTGAGCGGCACACTGCTGTGGCAGCTGACATGGGTCCGATCTC |
| | | AGCAGCCTGTGCAGTCTCCTCAGGCCGTGATTCTGAGAGAAGGCGAG |
| | | GACGCCGTGATCAACTGCAGCAGCTCTAAGGCCCTGTACAGCGTGCA |
| | | CTGGTACAGACAGAAGCACGGCGAGGCCCCTGTGTTCCTGATGATCC |
| | | TGCTGAAAGGCGGCGAGCAGAAGGGCCACGAGAAGATCAGCGCCAG |
| | | CTTCAACGAGAAGAAGCAGCAGTCCAGCCTGTACCTGACAGCCAGC |
| | | CAGCTGAGCTACAGCGGCACCTACTTTTGTGGCACCGCCTGGATCAA |
| | | CGACTACAAGCTGTCTTTCGGAGCCGGCACCACAGTGACAGTGCGGG |
| | | CCAATATTCAGAACCCCGATCCTGCCGTGTACCAGCTGAGAGACAGC |
| | | AAGAGCAGCGACAAGAGCGTGTGCCTGTTCACCGACTTCGACAGCC |
| | | AGACCAACGTGTCCCAGAGCAAGGACAGCGACGTGTACATCACCGA |
| | | TAAGACTGTGCTGGACATGCGGAGCATGGACTTCAAGAGCAACAGC |
| | | GCCGTGGCCTGGTCCAACAAGAGCGATTTCGCCTGCGCCAACGCCTT |
| | | CAACAACAGCATTATCCCCGAGGACACATTCTTCCCAAGTCCTGAGA |
| | | GCAGCTGCGACGTGAAGCTGGTGGAAAAGAGCTTCGAGACAGACAC |
| | | CAACCTGAACTTCCAGAACCTGAGCGTGATCGGCTTCAGAATCCTGC |
| | | TGCTCAAGGTGGCCGGCTTCAACCTGCTGATGACCCTGAGACTGTGG |
| | | TCCAGCTAACCTCGACTGTGCCTTCTAGTTGCCAGCCATCTGTTGTTT |
| | | GCCCCTCCCCCGTGCCTTCCTTGACCCTGGAAGGTGCCACTCCCACTG |
| | | TCCTTTCCTAATAAAATGAGGAAATTGCATCGCATTGTCTGAGTAGG |
| | | TGTCATTCTATTCTGGGGGGTGGGGTGGGGCAGGACAGCAAGGGGG |
| | | AGGATTGGGAAGACAATAGCAGGCATGCTGGGGATGCGGTGGGCTC |
| | | TATGGCTTCTGAGGCGGAAAGAACCAGCTGGGGCTCTAGGGGGTAT |
| | | CCCCACTAGTCGTGTACCAGCTGAGAGACTCTAAATCCAGTGACAAG |
| | | TCTGTCTGCCTATTCACCGATTTTGATTCTCAAACAAATGTGTCACAA |
| | | AGTAAGGATTCTGATGTGTATATCACAGACAAAACTGTGCTAGACAT |
| | | GAGGTCTATGGACTTCAAGAGCAACAGTGCTGTGGCCTGGAGCAAC |
| | | AAATCTGACTTTGCATGTGCAAACGCCTTCAACAACAGCATTATTCC |
| | | AGAAGACACCTTCTTCCCCAGCCCAGgtaagggcagctttggtgcct |
| | | tcgcaggctgtttccttgcttcaggaatggccaggttctgcccagagctc |
| | | tggtcaatgatgtctaaaactcctctgattggtggtctcggc |
| | | cttatccattgccaccaaaaccctctttttactaagaaacagtgagc |
| | | cttgttctggcagtccagagaatgacacgggaaaaaagcagatgaag |
| | | agaaggtggcaggagagggcacgtggcccagcctcagtctct |
| TCR P chain | SEQ ID | MGSWTLCCVSLCILVAKHTDAGVIQSPRHEVTEMGQEVTLRCKPISGHD |
| pINT1066 | NO: 10 | YLFWYRQTMMRGLELLIYFNNNVPIDDSGMPEDRFSAKMPNASFSTLKI |
| | | QPSEPRDSAVYFCASRKTGGYSNQPQHFGDGTRLSILEDLKNVFPPEVA |
| | | VFEPSEAEISHTQKATLVCLATGFYPDHVELSWWVNGKEVHSGVSTDP |
| | | QPLKEQPALNDSRYCLSSRLRVSATFWQNPRNHFRCQVQFYGLSENDE |
| | | WTQDRAKPVTQIVSAEAWGRADCGFTSESYQQGVLSATILYEILLGKAT |
| | | LYAVLVSALVLMAMVKRKDSRG |
| TCR a chain | SEQ ID | METLLKVLSGTLLWQLTWVRSQQPVQSPQAVILREGEDAVINCSSSKAL |
| pINT1066 | NO: 11 | YSVHWYRQKHGEAPVFLMILLKGGEQKGHEKISASFNEKKQQSSLYLT |
| | | ASQLSYSGTYFCGTAWINDYKLSFGAGTTVTVRANIQNPDPAVYQLRDS |
| | | KSSDKSVCLFTDFDSQTNVSQSKDSDVYITDKTVLDMRSMDFKSNSAV |
| | | AWSNKSDFACANAFNNSIIPEDTFFPSPESSCDVKLVEKSFETDTNLNFQ |
| | | NLSVIGFRILLLKVAGFNLLMTLRLWSS* |
| TCRp- | SEQ ID | MGSWTLCCVSLCILVAKHTDAGVIQSPRHEVTEMGQEVTLRCKPISGHD |
| linker-a | NO: 12 | YLFWYRQTMMRGLELLIYFNNNVPIDDSGMPEDRFSAKMPNASFSTLKI |
| configuration | | QPSEPRDSAVYFCASRKTGGYSNQPQHFGDGTRLSILEDLKNVFPPEVA |
| pINT1066 | | VFEPSEAEISHTQKATLVCLATGFYPDHVELSWWVNGKEVHSGVSTDP |
| | | QPLKEQPALNDSRYCLSSRLRVSATFWQNPRNHFRCQVQFYGLSENDE |
| | | WTQDRAKPVTQIVSAEAWGRADCGFTSESYQQGVLSATILYEILLGKAT |
| | | LYAVLVSALVLMAMVKRKDSRGGSGATNFSLLKQAGDVEENPGPMET |
| | | LLKVLSGTLLWQLTWVRSQQPVQSPQAVILREGEDAVINCSSSKALYSV |
| | | HWYRQKHGEAPVFLMILLKGGEQKGHEKISASFNEKKQQSSLYLTASQ |
| | | LSYSGTYFCGTAWINDYKLSFGAGTTVTVRANIQNPDPAVYQLRDSKSS |
| | | DKSVCLFTDFDSQTNVSQSKDSDVYITDKTVLDMRSMDFKSNSAVAWS |
| | | NKSDFACANAFNNSIIPEDTFFPSPESSCDVKLVEKSFETDTNLNFQNLSV |
| | | IGFRILLLKVAGFNLLMTLRLWSS* |
| Full HDRT | SEQ ID | GAGGGCCGCGGCAGCCTGCTGACCTGCGGCGACGTGGAGGAGAAtCC |
| template - | NO: 13 | CGGCCCCATGgtgAGCAAGGGCGAGGAGCTGTTCACCGGGGTGGTGC |
| GFP T2A | | CCATCCTGGTCGAGCTGGACGGCGACGTAAACGGCCACAAGTTCAG |
| insert | | CGTGTCCGGCGAGGGCGAGGGCGATGCCACCTACGGCAAGCTGACC |
| GFP: | | CTGAAGTTCATCTGCACCACCGGCAAGCTGCCCGTGCCCTGGCCCAC |
| P00894 | | CCTCGTGACCACCCTGACCTACGGCGTGCAGTGCTTCAGCCGCTACC |
| | | CCGACCACATGAAGCAGCACGACTTCTTCAAGTCCGCCATGCCCGAA |
| | | GGCTACGTCCAGGAGCGCACCATCTTCTTCAAGGACGACGGCAACTA |
| | | CAAGACCCGCGCCGAGGTGAAGTTCGAGGGCGACACCCTGGTGAAC |
| | | CGCATCGAGCTGAAGGGCATCGACTTCAAGGAGGACGGCAACATCC |
| | | TGGGGCACAAGCTGGAGTACAACTACAACAGCCACAACGTCTATAT |
| | | CATGGCCGACAAGCAGAAGAACGGCATCAAGGTGAACTTCAAGATC |
| | | CGCCACAACATCGAGGACGGCAGCGTGCAGCTCGCCGACCACTACC |
| | | AGCAGAACACCCCCATCGGCGACGGCCCCGTGCTGCTGCCCGACAA |
| | | CCACTACCTGAGCACCCAGTCCGCCCTGAGCAAAGACCCCAACGAG |
| | | AAGCGCGATCACATGGTCCTGCTGGAGTTCGTGACCGCCGCCGGGAT |
| | | CACTCTCGGCATGGACGAGCTGTACAAGTAAcctCGACTGTGCCTTCT |
| | | AGTTGCCAGCCATCTGTTGTTTGCCCCTCCCCCGTGCCTTCCTTGACC |
| | | CTGGAAGGTGCCACTCCCACTGTCCTTTCCTAATAAAATGAGGAAAT |
| | | TGCATCGCATTGTCTGAGTAGGTGTCATTCTATTCTGGGGGGTGGGG |
| | | TGGGGCAGGACAGCAAGGGGGAGGATTGGGAAGACAATAGCAGGC |
| | | ATGCTGGGGATGCGGTGGGCTCTATGGcttctgaggcggaaagaaccagctggggctct |
| | | agggggtatccccACTAGTCGTGTACCAGCTGAGAGACTCTAAATCCAGTG |
| | | ACAAGTCTGTCTGCCTATTCACCGATTTTGATTCTCAAACAAATGTGT |
| | | CACAAAGTAAGGATTCTGATGTGTATATCACAGACAAAACTGTGCTA |
| | | GACATGAGGTCTATGGACTTCAAGAGCAACAGTGCTGTGGCCTGGA |
| | | GCAACAAATCTGACTTTGCATGTGCAAACGCCTTCAACAACAGCATT |
| | | ATTCCAGAAGACACCTTCTTCCCCAGCCCAGgtaagggcagctttggtgccttcgca |
| | | ggctgtttccttgcttcaggaatggccaggttctgcccagagctctggtcaatgatg |
| | | tctaaaactcctctgattggtggtctcggccttatccattgccaccaaaaccctctt |
| | | tttactaagaaacagtgagccttgttctggcagtccagagaatg |
| | | acacgggaaaaaagcagatgaagagaaggtggcaggagagggcacgtgg |
| | | cccagcctcagtctct |
| eGFP ORF | SEQ ID | ATGGTGAGCAAGGGCGAGGAGCTGTTCACCGGGGTGGTGCCCATCC |
| GFP: | NO: 14 | TGGTCGAGCTGGACGGCGACGTAAACGGCCACAAGTTCAGCGTGTC |
| P00894, | | CGGCGAGGGCGAGGGCGATGCCACCTACGGCAAGCTGACCCTGAAG |
| GFP PO1018 | | TTCATCTGCACCACCGGCAAGCTGCCCGTGCCCTGGCCCACCCTCGT |
| | | GACCACCCTGACCTACGGCGTGCAGTGCTTCAGCCGCTACCCCGACC |
| | | ACATGAAGCAGCACGACTTCTTCAAGTCCGCCATGCCCGAAGGCTAC |
| | | GTCCAGGAGCGCACCATCTTCTTCAAGGACGACGGCAACTACAAGA |
| | | CCCGCGCCGAGGTGAAGTTCGAGGGCGACACCCTGGTGAACCGCAT |
| | | CGAGCTGAAGGGCATCGACTTCAAGGAGGACGGCAACATCCTGGGG |
| | | CACAAGCTGGAGTACAACTACAACAGCCACAACGTCTATATCATGGC |
| | | CGACAAGCAGAAGAACGGCATCAAGGTGAACTTCAAGATCCGCCAC |
| | | AACATCGAGGACGGCAGCGTGCAGCTCGCCGACCACTACCAGCAGA |
| | | ACACCCCCATCGGCGACGGCCCCGTGCTGCTGCCCGACAACCACTAC |
| | | CTGAGCACCCAGTCCGCCCTGAGCAAAGACCCCAACGAGAAGCGCG |
| | | ATCACATGGTCCTGCTGGAGTTCGTGACCGCCGCCGGGATCACTCTC |
| | | GGCATGGACGAGCTGTACAAGTAA |
| Full HDRT | SEQ ID | agatcttaatcttctgggtttccgttttctcgaatgaaa |
| template- | NO: 15 | aatgcaggtccgagcagttaactggctggggcaccattagcaagtcactt |
| GFP with | | agcatctctggggccagtctgcaaagcgagggggcagccttaatgtgcct |
| B2M | | ccagcctgaagtcctagaatgagcgcccggtgtcccaagctggggcgcgc |
| homology | | accccagatcggagggcgccgatgtacagacagcaaactcacccagtcta |
| arms | | gtgcatgccttcttaaacatcacgagactctaagaaaaggaaactgaaaa |
| GFP: | | cgggaaagtccctctctctaacctggcactgcgtcgctggcttggagaca |
| P01018 | | ggtgacggtccctgcgggccttgtcctgattggctgggcacgcgtttaat |
| | | ataagtggaggcgtcgcgctggcgggcattcctgaagctgacagcattcg |
| | | ggccgagaGGCTC |
| | | CGGTGCCCGTCAGTGGGCAGAGCGCACATCGCCCACAGTCCCCGAG |
| | | AAGTTGGGGGGAGGGGTCGGCAATTGAACCGGTGCCTAGAGAAGGT |
| | | GGCGCGGGGTAAACTGGGAAAGTGATGTCGTGTACTGGCTCCGCCTT |
| | | TTTCCCGAGGGTGGGGGAGAACCGTATATAAGTGCAGTAGTCGCCGT |
| | | GAACGTTCTTTTTCGCAACGGGTTTGCCGCCAGAACACAGGTAAGTG |
| | | CCGTGTGTGGTTCCCGCGGGCCTGGCCTCTTTACGGGTTATGGCCCTT |
| | | GCGTGCCTTGAATTACTTCCACGCCCCTGGCTGCAGTACGTGATTCTT |
| | | GATCCCGAGCTTCGGGTTGGAAGTGGGTGGGAGAGTTCGAGGCCTTG |
| | | CGCTTAAGGAGCCCCTTCGCCTCGTGCTTGAGTTGAGGCCTGGCTTG |
| | | GGCGCTGGGGCCGCCGCGTGCGAATCTGGTGGCACCTTCGCGCCTGT |
| | | CTCGCTGCTTTCGATAAGTCTCTAGCCATTTAAAATTTTTGATGACCT |
| | | GCTGCGACGCTTTTTTTCTGGCAAGATAGTCTTGTAAATGCGGGCCA |
| | | AGATgTGCACACTGGTATTTCGGTTTTTGGGGCCGCGGGCGGCGACG |
| | | GGGCCCGTGCGTCCCAGCGCACATGTTCGGCGAGGCGGGGCCTGCG |
| | | AGCGCGGCCACCGAGAATCGGACGGGGGTAGTCTCAAGCTGGCCGG |
| | | CCTGCTCTGGTGCCTGGCCTCGCGCCGCCGTGTATCGCCCCGCCCTG |
| | | GGCGGCAAGGCTGGCCCGGTCGGCACCAGTTGCGTGAGCGGAAAGA |
| | | TGGCCGCTTCCCGGCCCTGCTGCAGGGAGCTCAAAATGGAGGACGC |
| | | GGCGCTCGGGAGAGCGGGCGGGTGAGTCACCCACACAAAGGAAAAG |
| | | GGCCTTTCCGTCCTCAGCCGTCGCTTCATGTGACTCCACGGAGTACC |
| | | GGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCA |
| | | CACTGAGTGGGTGGAGACTGAAGTTAGGCCAGCTTGGCACTTGATGT |
| | | AATTCTCCTTGGAATTTGCCCTTTTTGAGTTTGGATCTTGGTTCATTCT |
| | | CAAGCCTCAGACAGTGGTTCAAAGTTTTTTTCTTCCATTTCAGGTGTC |
| | | GTGAcggCCGGCCCCGCCACCatggtgAGCAAGGGCGAGGAGCTGTTCAC |
| | | CGGGGTGGTGCCCATCCTGGTCGAGCTGGACGGCGACGTAAACGGC |
| | | CACAAGTTCAGCGTGTCCGGCGAGGGCGAGGGCGATGCCACCTACG |
| | | GCAAGCTGACCCTGAAGTTCATCTGCACCACCGGCAAGCTGCCCGTG |
| | | CCCTGGCCCACCCTCGTGACCACCCTGACCTACGGCGTGCAGTGCTT |
| | | CAGCCGCTACCCCGACCACATGAAGCAGCACGACTTCTTCAAGTCCG |
| | | CCATGCCCGAAGGCTACGTCCAGGAGCGCACCATCTTCTTCAAGGAC |
| | | GACGGCAACTACAAGACCCGCGCCGAGGTGAAGTTCGAGGGCGACA |
| | | CCCTGGTGAACCGCATCGAGCTGAAGGGCATCGACTTCAAGGAGGA |
| | | CGGCAACATCCTGGGGCACAAGCTGGAGTACAACTACAACAGCCAC |
| | | AACGTCTATATCATGGCCGACAAGCAGAAGAACGGCATCAAGGTGA |
| | | ACTTCAAGATCCGCCACAACATCGAGGACGGCAGCGTGCAGCTCGC |
| | | CGACCACTACCAGCAGAACACCCCCATCGGCGACGGCCCCGTGCTGC |
| | | TGCCCGACAACCACTACCTGAGCACCCAGTCCGCCCTGAGCAAAGAC |
| | | CCCAACGAGAAGCGCGATCACATGGTCCTGCTGGAGTTCGTGACCGC |
| | | CGCCGGGATCACTCTCGGCATGGACGAGCTGTACAAGTAATCTAGAc |
| | | ctCGACTGTGCCTTCTAGTTGCCAGCCATCTGTTGTTTGCCCCTCCCCC |
| | | GTGCCTTCCTTGACCCTGGAAGGTGCCACTCCCACTGTCCTTTCCTAA |
| | | TAAAATGAGGAAATTGCATCGCATTGTCTGAGTAGGTGTCATTCTAT |
| | | TCTGGGGGGTGGGGTGGGGCAGGACAGCAAGGGGGAGGATTGGGA |
| | | AGACAATAGCAGGCATGCTGGGGATGCGGT |
| | | GGGCTCTATGGcttctgaggcggaaagaac |
| | | cagctggggctctagggggtatccccACTA |
| | | GTtgtctcgctccgtggccttagctgtgct |
| | | cgcgctactctctctttctggcctggaggc |
| | | tatccagcgtgagtctctcctaccctcccg |
| | | ctctggtccttcctctcccgctctgcaccc |
| | | tctgtggccctcgctgtgctctctcgctcc |
| | | gtgacttcccttctccaagttctccttggt |
| | | ggcccgccgtggggctagtccagggctgga |
| | | tctcggggaagcggcggggtggcctgggag |
| | | tggggaagggggtgcgcacccgggacgcgc |
| | | gctacttgcccctttcggcggggagcaggg |
| | | gagacctttggcctacggcgacgggagggt |
| | | cgggacaaagtttagggcgtcgataagcgt |
| | | cagagcgccgaggttgggggagggtttctc |
| | | ttccgctctttcgcggggcctctggctccc |
| | | ccagcgcagctggagtgggggacgggtagg |
| | | ct |
| Cas9 amino | SEQ ID | MDKKYSIGLDIGTNSVGWAVITDEYKVPSKKFKVLGNTDRHSIKKNLIG |
| acid | NO: 16 | ALLFDSGETAEATRLKRTARRRYTRRKNRICYLQEIFSNEMAKVDDSFF |
| sequence for | | HRLEESFLVEEDKKHERHPIFGNIVDEVAYHEKYPTIYHLRKKLVDSTD |
| RNP | | KADLRLIYLALAHMIKFRGHFLIEGDLNPDNSDVDKLFIQLVQTYNQLFE |
| “*” in this | | ENPINASGVDAKAILSARLSKSRRLENLIAQLPGEKKNGLFGNLIALSLG |
| sequence | | LTPNFKSNFDLAEDAKLQLSKDTYDDDLDNLLAQIGDQYADLFLAAKN |
| denotes a | | LSDAILLSDILRVNTEITKAPLSASMIKRYDEHHQDLTLLKALVRQQLPE |
| stop codon | | KYKEIFFDQSKNGYAGYIDGGASQEEFYKFIKPILEKMDGTEELLVKLNR |
| | | EDLLRKQRTFDNGSIPHQIHLGELHAILRRQEDFYPFLKDNREKIEKILTF |
| | | RIPYYVGPLARGNSRFAWMTRKSEETITPWNFEEVVDKGASAQSFIERM |
| | | TNFDKNLPNEKVLPKHSLLYEYFTVYNELTKVKYVTEGMRKPAFLSGE |
| | | QKKAIVDLLFKTNRKVTVKQLKEDYFKKIECFDSVEISGVEDRFNASLG |
| | | TYHDLLKIIKDKDFLDNEENEDILEDIVLTLTLFEDREMIEERLKTYAHLF |
| | | DDKVMKQLKRRRYTGWGRLSRKLINGIRDKQSGKTILDFLKSDGFANR |
| | | NFMQLIHDDSLTFKEDIQKAQVSGQGDSLHEHIANLAGSPAIKKGILQTV |
| | | KVVDELVKVMGRHKPENIVIEMARENQTTQKGQKNSRERMKRIEEGIK |
| | | ELGSQILKEHPVENTQLQNEKLYLYYLQNGRDMYVDQELDINRLSDYD |
| | | VDHIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVAQIL |
| | | DSRMNTKYDENDKLIREVKVITLKSKLVSDFRKDFQFYKVREINNYHHA |
| | | HDAYLNAVVGTALIKKYPKLESEFVYGDYKVYDVRKMIAKSEQEIGKA |
| | | TAKYFFYSNIMNFFKTEITLANGEIRKRPLIETNGETGEIVWDKGRDFAT |
| | | VRKVLSMPQVNIVKKTEVQTGGFSKESILPKRNSDKLIARKKDWDPKK |
| | | YGGFDSPTVAYSVLVVAKVEKGKSKKLKSVKELLGITIMERSSFEKNPID |
| | | FLEAKGYKEVKKDLIIKLPKYSLFELENGRKRMLASAGELQKGNELALP |
| | | SKYVNFLYLASHYEKLKGSPEDNEQKQLFVEQHKHYLDEIIEQISEFSKR |
| | | VILADANLDKVLSAYNKHRDKPIREQAENIIHLFTLTNLGAPAAFKYFDT |
| | | TIDRKRYTSTKEVLDATLIHQSITGLYETRIDLSQLGGDGGGSPKKKRKV |
| | | * |
| mRNA | SEQ ID | GGGAAGCUCAGAAUAAACGCUCAACUUUGGCCGGAUCUGCCACCA |
| encoding | NO: 17 | UGGAGGCCUCCCCCGCCUCCGGCCCCCGGCACCUGAUGGACCCCCA |
| BC22n with | | CAUCUUCACCUCCAACUUCAACAACGGCAUCGGCCGGCACAAGACC |
| Hibit tag | | UACCUGUGCUACGAGGUGGAGCGGCUGGACAACGGCACCUCCGUG |
| | | AAGAUGGACCAGCACCGGGGCUUCCUGCACAACCAGGCCAAGAAC |
| | | CUGCUGUGCGGCUUCUACGGCCGGCACGCCGAGCUGCGGUUCCUG |
| | | GACCUGGUGCCCUCCCUGCAGCUGGACCCCGCCCAGAUCUACCGGG |
| | | UGACCUGGUUCAUCUCCUGGUCCCCCUGCUUCUCCUGGGGCUGCGC |
| | | CGGCGAGGUGCGGGCCUUCCUGCAGGAGAACACCCACGUGCGGCU |
| | | GCGGAUCUUCGCCGCCCGGAUCUACGACUACGACCCCCUGUACAAG |
| | | GAGGCCCUGCAGAUGCUGCGGGACGCCGGCGCCCAGGUGUCCAUC |
| | | AUGACCUACGACGAGUUCAAGCACUGCUGGGACACCUUCGUGGAC |
| | | CACCAGGGCUGCCCCUUCCAGCCCUGGGACGGCCUGGACGAGCACU |
| | | CCCAGGCCCUGUCCGGCCGGCUGCGGGCCAUCCUGCAGAACCAGGG |
| | | CAACUCCGGCUCCGAGACCCCCGGCACCUCCGAGUCCGCCACCCCC |
| | | GAGUCCGACAAGAAGUACUCCAUCGGCCUGGCCAUCGGCACCAAC |
| | | UCCGUGGGCUGGGCCGUGAUCACCGACGAGUACAAGGUGCCCUCC |
| | | AAGAAGUUCAAGGUGCUGGGCAACACCGACCGGCACUCCAUCAAG |
| | | AAGAACCUGAUCGGCGCCCUGCUGUUCGACUCCGGCGAGACCGCCG |
| | | AGGCCACCCGGCUGAAGCGGACCGCCCGGCGGCGGUACACCCGGCG |
| | | GAAGAACCGGAUCUGCUACCUGCAGGAGAUCUUCUCCAACGAGAU |
| | | GGCCAAGGUGGACGACUCCUUCUUCCACCGGCUGGAGGAGUCCUU |
| | | CCUGGUGGAGGAGGACAAGAAGCACGAGCGGCACCCCAUCUUCGG |
| | | CAACAUCGUGGACGAGGUGGCCUACCACGAGAAGUACCCCACCAU |
| | | CUACCACCUGCGGAAGAAGCUGGUGGACUCCACCGACAAGGCCGA |
| | | CCUGCGGCUGAUCUACCUGGCCCUGGCCCACAUGAUCAAGUUCCGG |
| | | GGCCACUUCCUGAUCGAGGGCGACCUGAACCCCGACAACUCCGACG |
| | | UGGACAAGCUGUUCAUCCAGCUGGUGCAGACCUACAACCAGCUGU |
| | | UCGAGGAGAACCCCAUCAACGCCUCCGGCGUGGACGCCAAGGCCAU |
| | | CCUGUCCGCCCGGCUGUCCAAGUCCCGGCGGCUGGAGAACCUGAUC |
| | | GCCCAGCUGCCCGGCGAGAAGAAGAACGGCCUGUUCGGCAACCUG |
| | | AUCGCCCUGUCCCUGGGCCUGACCCCCAACUUCAAGUCCAACUUCG |
| | | ACCUGGCCGAGGACGCCAAGCUGCAGCUGUCCAAGGACACCUACG |
| | | ACGACGACCUGGACAACCUGCUGGCCCAGAUCGGCGACCAGUACGC |
| | | CGACCUGUUCCUGGCCGCCAAGAACCUGUCCGACGCCAUCCUGCUG |
| | | UCCGACAUCCUGCGGGUGAACACCGAGAUCACCAAGGCCCCCCUGU |
| | | CCGCCUCCAUGAUCAAGCGGUACGACGAGCACCACCAGGACCUGAC |
| | | CCUGCUGAAGGCCCUGGUGCGGCAGCAGCUGCCCGAGAAGUACAA |
| | | GGAGAUCUUCUUCGACCAGUCCAAGAACGGCUACGCCGGCUACAU |
| | | CGACGGCGGCGCCUCCCAGGAGGAGUUCUACAAGUUCAUCAAGCC |
| | | CAUCCUGGAGAAGAUGGACGGCACCGAGGAGCUGCUGGUGAAGCU |
| | | GAACCGGGAGGACCUGCUGCGGAAGCAGCGGACCUUCGACAACGG |
| | | CUCCAUCCCCCACCAGAUCCACCUGGGCGAGCUGCACGCCAUCCUG |
| | | CGGCGGCAGGAGGACUUCUACCCCUUCCUGAAGGACAACCGGGAG |
| | | AAGAUCGAGAAGAUCCUGACCUUCCGGAUCCCCUACUACGUGGGC |
| | | CCCCUGGCCCGGGGCAACUCCCGGUUCGCCUGGAUGACCCGGAAGU |
| | | CCGAGGAGACCAUCACCCCCUGGAACUUCGAGGAGGUGGUGGACA |
| | | AGGGCGCCUCCGCCCAGUCCUUCAUCGAGCGGAUGACCAACUUCGA |
| | | CAAGAACCUGCCCAACGAGAAGGUGCUGCCCAAGCACUCCCUGCUG |
| | | UACGAGUACUUCACCGUGUACAACGAGCUGACCAAGGUGAAGUAC |
| | | GUGACCGAGGGCAUGCGGAAGCCCGCCUUCCUGUCCGGCGAGCAG |
| | | AAGAAGGCCAUCGUGGACCUGCUGUUCAAGACCAACCGGAAGGUG |
| | | ACCGUGAAGCAGCUGAAGGAGGACUACUUCAAGAAGAUCGAGUGC |
| | | UUCGACUCCGUGGAGAUCUCCGGCGUGGAGGACCGGUUCAACGCC |
| | | UCCCUGGGCACCUACCACGACCUGCUGAAGAUCAUCAAGGACAAG |
| | | GACUUCCUGGACAACGAGGAGAACGAGGACAUCCUGGAGGACAUC |
| | | GUGCUGACCCUGACCCUGUUCGAGGACCGGGAGAUGAUCGAGGAG |
| | | CGGCUGAAGACCUACGCCCACCUGUUCGACGACAAGGUGAUGAAG |
| | | CAGCUGAAGCGGCGGCGGUACACCGGCUGGGGCCGGCUGUCCCGG |
| | | AAGCUGAUCAACGGCAUCCGGGACAAGCAGUCCGGCAAGACCAUC |
| | | CUGGACUUCCUGAAGUCCGACGGCUUCGCCAACCGGAACUUCAUG |
| | | CAGCUGAUCCACGACGACUCCCUGACCUUCAAGGAGGACAUCCAG |
| | | AAGGCCCAGGUGUCCGGCCAGGGCGACUCCCUGCACGAGCACAUCG |
| | | CCAACCUGGCCGGCUCCCCCGCCAUCAAGAAGGGCAUCCUGCAGAC |
| | | CGUGAAGGUGGUGGACGAGCUGGUGAAGGUGAUGGGCCGGCACAA |
| | | GCCCGAGAACAUCGUGAUCGAGAUGGCCCGGGAGAACCAGACCAC |
| | | CCAGAAGGGCCAGAAGAACUCCCGGGAGCGGAUGAAGCGGAUCGA |
| | | GGAGGGCAUCAAGGAGCUGGGCUCCCAGAUCCUGAAGGAGCACCC |
| | | CGUGGAGAACACCCAGCUGCAGAACGAGAAGCUGUACCUGUACUA |
| | | CCUGCAGAACGGCCGGGACAUGUACGUGGACCAGGAGCUGGACAU |
| | | CAACCGGCUGUCCGACUACGACGUGGACCACAUCGUGCCCCAGUCC |
| | | UUCCUGAAGGACGACUCCAUCGACAACAAGGUGCUGACCCGGUCC |
| | | GACAAGAACCGGGGCAAGUCCGACAACGUGCCCUCCGAGGAGGUG |
| | | GUGAAGAAGAUGAAGAACUACUGGCGGCAGCUGCUGAACGCCAAG |
| | | CUGAUCACCCAGCGGAAGUUCGACAACCUGACCAAGGCCGAGCGG |
| | | GGCGGCCUGUCCGAGCUGGACAAGGCCGGCUUCAUCAAGCGGCAG |
| | | CUGGUGGAGACCCGGCAGAUCACCAAGCACGUGGCCCAGAUCCUG |
| | | GACUCCCGGAUGAACACCAAGUACGACGAGAACGACAAGCUGAUC |
| | | CGGGAGGUGAAGGUGAUCACCCUGAAGUCCAAGCUGGUGUCCGAC |
| | | UUCCGGAAGGACUUCCAGUUCUACAAGGUGCGGGAGAUCAACAAC |
| | | UACCACCACGCCCACGACGCCUACCUGAACGCCGUGGUGGGCACCG |
| | | CCCUGAUCAAGAAGUACCCCAAGCUGGAGUCCGAGUUCGUGUACG |
| | | GCGACUACAAGGUGUACGACGUGCGGAAGAUGAUCGCCAAGUCCG |
| | | AGCAGGAGAUCGGCAAGGCCACCGCCAAGUACUUCUUCUACUCCA |
| | | ACAUCAUGAACUUCUUCAAGACCGAGAUCACCCUGGCCAACGGCG |
| | | AGAUCCGGAAGCGGCCCCUGAUCGAGACCAACGGCGAGACCGGCG |
| | | AGAUCGUGUGGGACAAGGGCCGGGACUUCGCCACCGUGCGGAAGG |
| | | UGCUGUCCAUGCCCCAGGUGAACAUCGUGAAGAAGACCGAGGUGC |
| | | AGACCGGCGGCUUCUCCAAGGAGUCCAUCCUGCCCAAGCGGAACUC |
| | | CGACAAGCUGAUCGCCCGGAAGAAGGACUGGGACCCCAAGAAGUA |
| | | CGGCGGCUUCGACUCCCCCACCGUGGCCUACUCCGUGCUGGUGGUG |
| | | GCCAAGGUGGAGAAGGGCAAGUCCAAGAAGCUGAAGUCCGUGAAG |
| | | GAGCUGCUGGGCAUCACCAUCAUGGAGCGGUCCUCCUUCGAGAAG |
| | | AACCCCAUCGACUUCCUGGAGGCCAAGGGCUACAAGGAGGUGAAG |
| | | AAGGACCUGAUCAUCAAGCUGCCCAAGUACUCCCUGUUCGAGCUG |
| | | GAGAACGGCCGGAAGCGGAUGCUGGCCUCCGCCGGCGAGCUGCAG |
| | | AAGGGCAACGAGCUGGCCCUGCCCUCCAAGUACGUGAACUUCCUG |
| | | UACCUGGCCUCCCACUACGAGAAGCUGAAGGGCUCCCCCGAGGACA |
| | | ACGAGCAGAAGCAGCUGUUCGUGGAGCAGCACAAGCACUACCUGG |
| | | ACGAGAUCAUCGAGCAGAUCUCCGAGUUCUCCAAGCGGGUGAUCC |
| | | UGGCCGACGCCAACCUGGACAAGGUGCUGUCCGCCUACAACAAGC |
| | | ACCGGGACAAGCCCAUCCGGGAGCAGGCCGAGAACAUCAUCCACCU |
| | | GUUCACCCUGACCAACCUGGGCGCCCCCGCCGCCUUCAAGUACUUC |
| | | GACACCACCAUCGACCGGAAGCGGUACACCUCCACCAAGGAGGUGC |
| | | UGGACGCCACCCUGAUCCACCAGUCCAUCACCGGCCUGUACGAGAC |
| | | CCGGAUCGACCUGUCCCAGCUGGGCGGCGACGGCGGCGGCUCCCCC |
| | | AAGAAGAAGCGGAAGGUGUCCGAGUCCGCCACCCCCGAGUCCGUG |
| | | UCCGGCUGGCGGCUGUUCAAGAAGAUCUCCUGACUAGCACCAGCC |
| | | UCAAGAACACCCGAAUGGAGUCUCUAAGCUACAUAAUACCAACUU |
| | | ACACUUUACAAAAUGUUGUCCCCCAAAAUGUAGCCAUUCGUAUCU |
| | | GCUCCUAAUAAAAAGAAAGUUUCUUCACAUUCUCUCGAGAAAAAA |
| | | AAAAAAUGGAAAAAAAAAAAACGGAAAAAAAAAAAAGGUAAAAA |
| | | AAAAAAAUAUAAAAAAAAAAAACAUAAAAAAAAAAAACGAAAAA |
| | | AAAAAAACGUAAAAAAAAAAAACUCAAAAAAAAAAAAGAUAAAA |
| | | AAAAAAAACCUAAAAAAAAAAAAUGUAAAAAAAAAAAAGGGAAA |
| | | AAAAAAAAACGCAAAAAAAAAAAACACAAAAAAAAAAAAUGCAAA |
| | | AAAAAAAAAUCGAAAAAAAAAAAAUCUAAAAAAAAAAAACGAAA |
| | | AAAAAAAAACCCAAAAAAAAAAAAGACAAAAAAAAAAAAUAGAAA |
| | | AAAAAAAAAGUUAAAAAAAAAAAACUGAAAAAAAAAAAAUUUAA |
| | | AAAAAAAAAAUCUAG |
| Open | SEQ ID | AUGGAGGCCUCCCCCGCCUCCGGCCCCCGGCACCUGAUGGACCCCC |
| reading | NO: 18 | ACAUCUUCACCUCCAACUUCAACAACGGCAUCGGCCGGCACAAGAC |
| frame for | | CUACCUGUGCUACGAGGUGGAGCGGCUGGACAACGGCACCUCCGU |
| BC22n with | | GAAGAUGGACCAGCACCGGGGCUUCCUGCACAACCAGGCCAAGAA |
| Hibit tag | | CCUGCUGUGCGGCUUCUACGGCCGGCACGCCGAGCUGCGGUUCCUG |
| | | GACCUGGUGCCCUCCCUGCAGCUGGACCCCGCCCAGAUCUACCGGG |
| | | UGACCUGGUUCAUCUCCUGGUCCCCCUGCUUCUCCUGGGGCUGCGC |
| | | CGGCGAGGUGCGGGCCUUCCUGCAGGAGAACACCCACGUGCGGCU |
| | | GCGGAUCUUCGCCGCCCGGAUCUACGACUACGACCCCCUGUACAAG |
| | | GAGGCCCUGCAGAUGCUGCGGGACGCCGGCGCCCAGGUGUCCAUC |
| | | AUGACCUACGACGAGUUCAAGCACUGCUGGGACACCUUCGUGGAC |
| | | CACCAGGGCUGCCCCUUCCAGCCCUGGGACGGCCUGGACGAGCACU |
| | | CCCAGGCCCUGUCCGGCCGGCUGCGGGCCAUCCUGCAGAACCAGGG |
| | | CAACUCCGGCUCCGAGACCCCCGGCACCUCCGAGUCCGCCACCCCC |
| | | GAGUCCGACAAGAAGUACUCCAUCGGCCUGGCCAUCGGCACCAAC |
| | | UCCGUGGGCUGGGCCGUGAUCACCGACGAGUACAAGGUGCCCUCC |
| | | AAGAAGUUCAAGGUGCUGGGCAACACCGACCGGCACUCCAUCAAG |
| | | AAGAACCUGAUCGGCGCCCUGCUGUUCGACUCCGGCGAGACCGCCG |
| | | AGGCCACCCGGCUGAAGCGGACCGCCCGGCGGCGGUACACCCGGCG |
| | | GAAGAACCGGAUCUGCUACCUGCAGGAGAUCUUCUCCAACGAGAU |
| | | GGCCAAGGUGGACGACUCCUUCUUCCACCGGCUGGAGGAGUCCUU |
| | | CCUGGUGGAGGAGGACAAGAAGCACGAGCGGCACCCCAUCUUCGG |
| | | CAACAUCGUGGACGAGGUGGCCUACCACGAGAAGUACCCCACCAU |
| | | CUACCACCUGCGGAAGAAGCUGGUGGACUCCACCGACAAGGCCGA |
| | | CCUGCGGCUGAUCUACCUGGCCCUGGCCCACAUGAUCAAGUUCCGG |
| | | GGCCACUUCCUGAUCGAGGGCGACCUGAACCCCGACAACUCCGACG |
| | | UGGACAAGCUGUUCAUCCAGCUGGUGCAGACCUACAACCAGCUGU |
| | | UCGAGGAGAACCCCAUCAACGCCUCCGGCGUGGACGCCAAGGCCAU |
| | | CCUGUCCGCCCGGCUGUCCAAGUCCCGGCGGCUGGAGAACCUGAUC |
| | | GCCCAGCUGCCCGGCGAGAAGAAGAACGGCCUGUUCGGCAACCUG |
| | | AUCGCCCUGUCCCUGGGCCUGACCCCCAACUUCAAGUCCAACUUCG |
| | | ACCUGGCCGAGGACGCCAAGCUGCAGCUGUCCAAGGACACCUACG |
| | | ACGACGACCUGGACAACCUGCUGGCCCAGAUCGGCGACCAGUACGC |
| | | CGACCUGUUCCUGGCCGCCAAGAACCUGUCCGACGCCAUCCUGCUG |
| | | UCCGACAUCCUGCGGGUGAACACCGAGAUCACCAAGGCCCCCCUGU |
| | | CCGCCUCCAUGAUCAAGCGGUACGACGAGCACCACCAGGACCUGAC |
| | | CCUGCUGAAGGCCCUGGUGCGGCAGCAGCUGCCCGAGAAGUACAA |
| | | GGAGAUCUUCUUCGACCAGUCCAAGAACGGCUACGCCGGCUACAU |
| | | CGACGGCGGCGCCUCCCAGGAGGAGUUCUACAAGUUCAUCAAGCC |
| | | CAUCCUGGAGAAGAUGGACGGCACCGAGGAGCUGCUGGUGAAGCU |
| | | GAACCGGGAGGACCUGCUGCGGAAGCAGCGGACCUUCGACAACGG |
| | | CUCCAUCCCCCACCAGAUCCACCUGGGCGAGCUGCACGCCAUCCUG |
| | | CGGCGGCAGGAGGACUUCUACCCCUUCCUGAAGGACAACCGGGAG |
| | | AAGAUCGAGAAGAUCCUGACCUUCCGGAUCCCCUACUACGUGGGC |
| | | CCCCUGGCCCGGGGCAACUCCCGGUUCGCCUGGAUGACCCGGAAGU |
| | | CCGAGGAGACCAUCACCCCCUGGAACUUCGAGGAGGUGGUGGACA |
| | | AGGGCGCCUCCGCCCAGUCCUUCAUCGAGCGGAUGACCAACUUCGA |
| | | CAAGAACCUGCCCAACGAGAAGGUGCUGCCCAAGCACUCCCUGCUG |
| | | UACGAGUACUUCACCGUGUACAACGAGCUGACCAAGGUGAAGUAC |
| | | GUGACCGAGGGCAUGCGGAAGCCCGCCUUCCUGUCCGGCGAGCAG |
| | | AAGAAGGCCAUCGUGGACCUGCUGUUCAAGACCAACCGGAAGGUG |
| | | ACCGUGAAGCAGCUGAAGGAGGACUACUUCAAGAAGAUCGAGUGC |
| | | UUCGACUCCGUGGAGAUCUCCGGCGUGGAGGACCGGUUCAACGCC |
| | | UCCCUGGGCACCUACCACGACCUGCUGAAGAUCAUCAAGGACAAG |
| | | GACUUCCUGGACAACGAGGAGAACGAGGACAUCCUGGAGGACAUC |
| | | GUGCUGACCCUGACCCUGUUCGAGGACCGGGAGAUGAUCGAGGAG |
| | | CGGCUGAAGACCUACGCCCACCUGUUCGACGACAAGGUGAUGAAG |
| | | CAGCUGAAGCGGCGGCGGUACACCGGCUGGGGCCGGCUGUCCCGG |
| | | AAGCUGAUCAACGGCAUCCGGGACAAGCAGUCCGGCAAGACCAUC |
| | | CUGGACUUCCUGAAGUCCGACGGCUUCGCCAACCGGAACUUCAUG |
| | | CAGCUGAUCCACGACGACUCCCUGACCUUCAAGGAGGACAUCCAG |
| | | AAGGCCCAGGUGUCCGGCCAGGGCGACUCCCUGCACGAGCACAUCG |
| | | CCAACCUGGCCGGCUCCCCCGCCAUCAAGAAGGGCAUCCUGCAGAC |
| | | CGUGAAGGUGGUGGACGAGCUGGUGAAGGUGAUGGGCCGGCACAA |
| | | GCCCGAGAACAUCGUGAUCGAGAUGGCCCGGGAGAACCAGACCAC |
| | | CCAGAAGGGCCAGAAGAACUCCCGGGAGCGGAUGAAGCGGAUCGA |
| | | GGAGGGCAUCAAGGAGCUGGGCUCCCAGAUCCUGAAGGAGCACCC |
| | | CGUGGAGAACACCCAGCUGCAGAACGAGAAGCUGUACCUGUACUA |
| | | CCUGCAGAACGGCCGGGACAUGUACGUGGACCAGGAGCUGGACAU |
| | | CAACCGGCUGUCCGACUACGACGUGGACCACAUCGUGCCCCAGUCC |
| | | UUCCUGAAGGACGACUCCAUCGACAACAAGGUGCUGACCCGGUCC |
| | | GACAAGAACCGGGGCAAGUCCGACAACGUGCCCUCCGAGGAGGUG |
| | | GUGAAGAAGAUGAAGAACUACUGGCGGCAGCUGCUGAACGCCAAG |
| | | CUGAUCACCCAGCGGAAGUUCGACAACCUGACCAAGGCCGAGCGG |
| | | GGCGGCCUGUCCGAGCUGGACAAGGCCGGCUUCAUCAAGCGGCAG |
| | | CUGGUGGAGACCCGGCAGAUCACCAAGCACGUGGCCCAGAUCCUG |
| | | GACUCCCGGAUGAACACCAAGUACGACGAGAACGACAAGCUGAUC |
| | | CGGGAGGUGAAGGUGAUCACCCUGAAGUCCAAGCUGGUGUCCGAC |
| | | UUCCGGAAGGACUUCCAGUUCUACAAGGUGCGGGAGAUCAACAAC |
| | | UACCACCACGCCCACGACGCCUACCUGAACGCCGUGGUGGGCACCG |
| | | CCCUGAUCAAGAAGUACCCCAAGCUGGAGUCCGAGUUCGUGUACG |
| | | GCGACUACAAGGUGUACGACGUGCGGAAGAUGAUCGCCAAGUCCG |
| | | AGCAGGAGAUCGGCAAGGCCACCGCCAAGUACUUCUUCUACUCCA |
| | | ACAUCAUGAACUUCUUCAAGACCGAGAUCACCCUGGCCAACGGCG |
| | | AGAUCCGGAAGCGGCCCCUGAUCGAGACCAACGGCGAGACCGGCG |
| | | AGAUCGUGUGGGACAAGGGCCGGGACUUCGCCACCGUGCGGAAGG |
| | | UGCUGUCCAUGCCCCAGGUGAACAUCGUGAAGAAGACCGAGGUGC |
| | | AGACCGGCGGCUUCUCCAAGGAGUCCAUCCUGCCCAAGCGGAACUC |
| | | CGACAAGCUGAUCGCCCGGAAGAAGGACUGGGACCCCAAGAAGUA |
| | | CGGCGGCUUCGACUCCCCCACCGUGGCCUACUCCGUGCUGGUGGUG |
| | | GCCAAGGUGGAGAAGGGCAAGUCCAAGAAGCUGAAGUCCGUGAAG |
| | | GAGCUGCUGGGCAUCACCAUCAUGGAGCGGUCCUCCUUCGAGAAG |
| | | AACCCCAUCGACUUCCUGGAGGCCAAGGGCUACAAGGAGGUGAAG |
| | | AAGGACCUGAUCAUCAAGCUGCCCAAGUACUCCCUGUUCGAGCUG |
| | | GAGAACGGCCGGAAGCGGAUGCUGGCCUCCGCCGGCGAGCUGCAG |
| | | AAGGGCAACGAGCUGGCCCUGCCCUCCAAGUACGUGAACUUCCUG |
| | | UACCUGGCCUCCCACUACGAGAAGCUGAAGGGCUCCCCCGAGGACA |
| | | ACGAGCAGAAGCAGCUGUUCGUGGAGCAGCACAAGCACUACCUGG |
| | | ACGAGAUCAUCGAGCAGAUCUCCGAGUUCUCCAAGCGGGUGAUCC |
| | | UGGCCGACGCCAACCUGGACAAGGUGCUGUCCGCCUACAACAAGC |
| | | ACCGGGACAAGCCCAUCCGGGAGCAGGCCGAGAACAUCAUCCACCU |
| | | GUUCACCCUGACCAACCUGGGCGCCCCCGCCGCCUUCAAGUACUUC |
| | | GACACCACCAUCGACCGGAAGCGGUACACCUCCACCAAGGAGGUGC |
| | | UGGACGCCACCCUGAUCCACCAGUCCAUCACCGGCCUGUACGAGAC |
| | | CCGGAUCGACCUGUCCCAGCUGGGCGGCGACGGCGGCGGCUCCCCC |
| | | AAGAAGAAGCGGAAGGUGUCCGAGUCCGCCACCCCCGAGUCCGUG |
| | | UCCGGCUGGCGGCUGUUCAAGAAGAUCUCCUGA |
| Amino acid | SEQ ID | MEASPASGPRHLMDPHIFTSNFNNGIGRHKTYLCYEVERLDNGTSVKM |
| sequence for | NO: 19 | DQHRGFLHNQAKNLLCGFYGRHAELRFLDLVPSLQLDPAQIYRVTWFIS |
| BC22n with | | WSPCFSWGCAGEVRAFLQENTHVRLRIFAARIYDYDPLYKEALQMLRD |
| Hibit tag | | AGAQVSIMTYDEFKHCWDTFVDHQGCPFQPWDGLDEHSQALSGRLRAI |
| | | LQNQGNSGSETPGTSESATPESDKKYSIGLAIGTNSVGWAVITDEYKVPS |
| | | KKFKVLGNTDRHSIKKNLIGALLFDSGETAEATRLKRTARRRYTRRKNR |
| | | ICYLQEIFSNEMAKVDDSFFHRLEESFLVEEDKKHERHPIFGNIVDEVAY |
| | | HEKYPTIYHLRKKLVDSTDKADLRLIYLALAHMIKFRGHFLIEGDLNPD |
| | | NSDVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDDLD |
| | | NLLAQIGDQYADLFLAAKNLSDAILLSDILRVNTEITKAPLSASMIKRYD |
| | | EHHQDLTLLKALVRQQLPEKYKEIFFDQSKNGYAGYIDGGASQEEFYKF |
| | | IKPILEKMDGTEELLVKLNREDLLRKQRTFDNGSIPHQIHLGELHAILRR |
| | | QEDFYPFLKDNREKIEKILTFRIPYYVGPLARGNSRFAWMTRKSEETITP |
| | | WNFEEVVDKGASAQSFIERMTNFDKNLPNEKVLPKHSLLYEYFTVYNE |
| | | LTKVKYVTEGMRKPAFLSGEQKKAIVDLLFKTNRKVTVKQLKEDYFKK |
| | | lECFDSVEISGVEDRFNASLGTYHDLLKIIKDKDFLDNEENEDILEDIVLT |
| | | LTLFEDREMIEERLKTYAHLFDDKVMKQLKRRRYTGWGRLSRKLINGIR |
| | | DKQSGKTILDFLKSDGFANRNFMQLIHDDSLTFKEDIQKAQVSGQGDSL |
| | | HEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQN |
| | | GRDMYVDQELDINRLSDYDVDHIVPQSFLKDDSIDNKVLTRSDKNRGK |
| | | SDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLV |
| | | SDFRKDFQFYKVREINNYHHAHDAYLNAVVGTALIKKYPKLESEFVYG |
| | | DYKVYDVRKMIAKSEQEIGKATAKYFFYSNIMNFFKTEITLANGEIRKRP |
| | | LIETNGETGEIVWDKGRDFATVRKVLSMPQVNIVKKTEVQTGGFSKESI |
| | | LPKRNSDKLIARKKDWDPKKYGGFDSPTVAYSVLVVAKVEKGKSKKL |
| | | KSVKELLGITIMERSSFEKNPIDFLEAKGYKEVKKDLIIKLPKYSLFELEN |
| | | GRKRMLASAGELQKGNELALPSKYVNFLYLASHYEKLKGSPEDNEQKQ |
| | | LFVEQHKHYLDEIIEQISEFSKRVILADANLDKVLSAYNKHRDKPIREQA |
| | | ENIIHLFTLTNLGAPAAFKYFDTTIDRKRYTSTKEVLDATLIHQSITGLYE |
| | | TRIDLSQLGGDGGGSPKKKRKVSESATPESVSGWRLFKKIS |
| mRNA | SEQ ID | GGGAGACCCAAGCUGGCUAGCUCCCGCAGUCGGCGUCCAGCGGCUC |
| encoding | NO: 20 | UGCUUGUUCGUGUGUGUGUCGUUGCAGGCCUUAUUCGGAUCCGCC |
| UGI | | ACCAUGGGACCGAAGAAGAAGAGAAAGGUCGGAGGAGGAAGCACA |
| | | AACCUGUCGGACAUCAUCGAAAAGGAAACAGGAAAGCAGCUGGUC |
| | | AUCCAGGAAUCGAUCCUGAUGCUGCCGGAAGAAGUCGAAGAAGUC |
| | | AUCGGAAACAAGCCGGAAUCGGACAUCCUGGUCCACACAGCAUAC |
| | | GACGAAUCGACAGACGAAAACGUCAUGCUGCUGACAUCGGACGCA |
| | | CCGGAAUACAAGCCGUGGGCACUGGUCAUCCAGGACUCGAACGGA |
| | | GAAAACAAGAUCAAGAUGCUGUGAUAGUCUAGACAUCACAUUUAA |
| | | AAGCAUCUCAGCCUACCAUGAGAAUAAGAGAAAGAAAAUGAAGAU |
| | | CAAUAGCUUAUUCAUCUCUUUUUCUUUUUCGUUGGUGUAAAGCCA |
| | | ACACCCUGUCUAAAAAACAUAAAUUUCUUUAAUCAUUUUGCCUCU |
| | | UUUCUCUGUGCUUCAAUUAAUAAAAAAUGGAAAGAACCUCGAGUC |
| | | UAG |
| Open | SEQ ID | AUGGGACCGAAGAAGAAGAGAAAGGUCGGAGGAGGAAGCACAAAC |
| reading | NO: 21 | CUGUCGGACAUCAUCGAAAAGGAAACAGGAAAGCAGCUGGUCAUC |
| frame for | | CAGGAAUCGAUCCUGAUGCUGCCGGAAGAAGUCGAAGAAGUCAUC |
| UGI | | GGAAACAAGCCGGAAUCGGACAUCCUGGUCCACACAGCAUACGAC |
| | | GAAUCGACAGACGAAAACGUCAUGCUGCUGACAUCGGACGCACCG |
| | | GAAUACAAGCCGUGGGCACUGGUCAUCCAGGACUCGAACGGAGAA |
| | | AACAAGAUCAAGAUGCUGUGA |
| Amino acid | SEQ ID | MTNLSDIIEKETGKQLVIQESILMLPEEVEEVIGNKPESDILVHTAYDEST |
| sequence for | NO: 22 | DENVMLLTSDAPEYKPWALVIQDSNGENKIKMLSGGSKRTADGSEFESP |
| UGI | | KKKRKVE |

TABLE 90
List of lipids

| | | Com- | |
|---|---|---|---|
| Lipid | | pound | |
| ID | Filing | ID | Structure |
| Lipid B | WO2020/ 072605 A1 | Com- pound 1 | 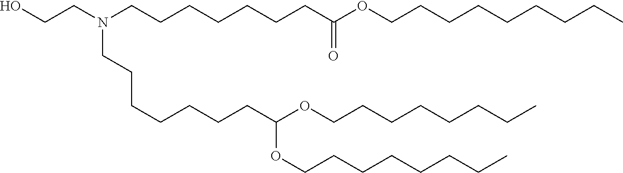 [SMILES: CCCCCCCCCOC(=O)CCCCCCCN(CCO)CCCCCCCC(OCCCCCCCC)OCCCCCCCC] |
| Lipid C | WO2020/ 072605 A1 | Com- pound 10 | 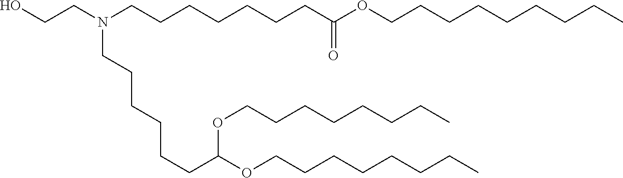 [SMILES: CCCCCCCCCOC(=O)CCCCCCCN(CCO)CCCCCCC(OCCCCCCCC)OCCCCCCCC] |
| Lipid D | WO2020/ 072605 A1 | Com- pound 18 | 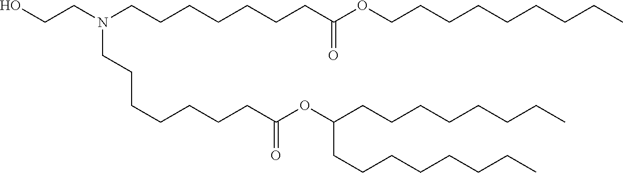 [SMILES: CCCCCCCCCOC(=O)CCCCCCCN(CCO)CCCCCCCC(=O)OC(CCCCCCCC)CCCCCCCC] |
| Lipid E | WO2020/ 118041 A1 | Com- pound 45 | 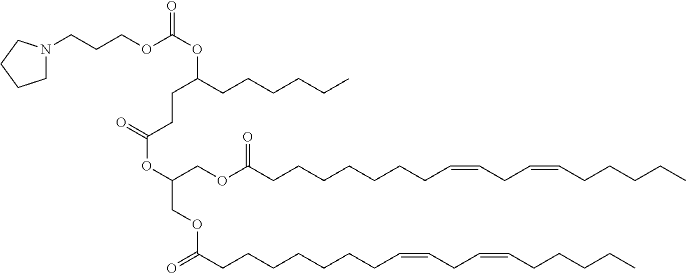 [SMILES: CCCCC/C=C\C/C=C\CCCCCCCC(=O)OCC(COC(=O)CCCCCCC/C=C\C/C=C\CCCCC)OC(=O)CCC(CCCCCC)OC(=O)OCCCN1CCCC1] |
| Lipid F | WO2020/ 118041 A1 | Com- pound 50 | 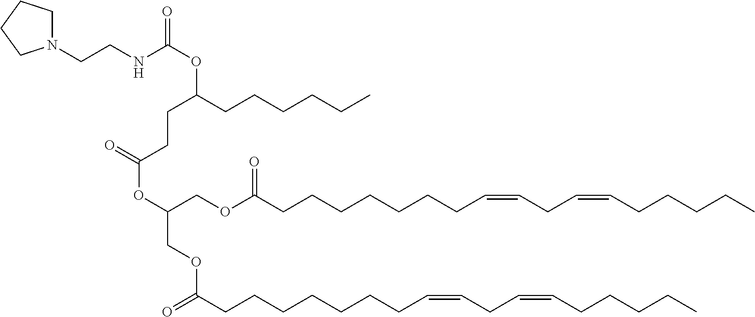 [SMILES: CCCCC/C=C\C/C=C\CCCCCCCC(=O)OCC(COC(=O)CCCCCCC/C=C\C/C=C\CCCCC)OC(=O)CCC(CCCCCC)OC(=O)NCCN1CCCC1] |
| Lipid G | WO2020/ 118041 A1 | Com- pound 85 | 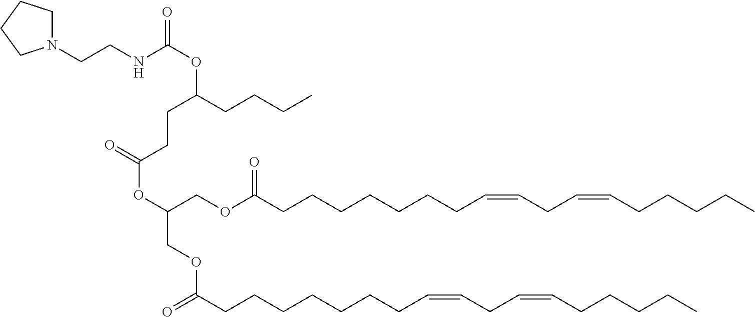 [SMILES: CCCCC/C=C\C/C=C\CCCCCCCC(=O)OCC(COC(=O)CCCCCCC/C=C\C/C=C\CCCCC)OC(=O)CCC(CCCC)OC(=O)NCCN1CCCC1] |
| Lipid H | WO2015/ 095340 A1 | S024 | 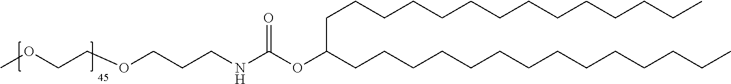 |
| Lipid J | WO2015/ 095340 A1 | S029 | 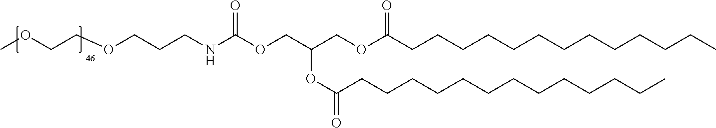 |

1. A cell population comprising edited cells comprising multiple genome edits per cell, wherein at least 50% of the cells in the cell population comprise at least two genome edits and
wherein: (i) fewer than 1%, fewer than 0.5%, fewer than 0.2%, or fewer than 0.1% of the cells in the cell population have a target-to-target translocation; or (ii) the cell population has less than 2 times the background level of reciprocal translocations, complex translocations, or off-target translocations; or
wherein the cell population is capable of expansion 50-fold ex vivo within 14 days in culture after initiation of editing; or
wherein: (i) fewer than 1%, fewer than 0.5%, fewer than 0.2%, or fewer than 0.1% of the cells in the cell population have a target-to-target translocation; or (ii) the cell population has less than 2 times the background level of reciprocal translocations, complex translocations, or off-target translocations, and wherein the cell population is capable of expansion 50-fold ex vivo within 14 days in culture after initiation of editing.

2-4. (canceled)

5. The cell population of claim 1, wherein at least one genome edit of the multiple genome edits is produced by a genome editing tool comprising an RNA-guided DNA binding agent.

6-14. (canceled)

15. The cell population of claim 1, wherein the cells are T cells.

16-18. (canceled)

19. The cell population of claim 1, wherein at least 95% of the cells in the cell population comprise a genome edit of an endogenous T cell receptor (TCR) sequence.

20. The cell population of claim 1, wherein a genome edit comprises insertion of an exogenous nucleic acid coding for a targeting ligand or an alternative antigen binding moiety, and wherein at least 70% of the cells of the cell population comprise an insertion of an exogenous nucleic acid into a target sequence.

21-25. (canceled)

26. A method of producing multiple genome edits in an in vitro-cultured cell, comprising the steps of:
a. contacting the cell in vitro with at least a first lipid nanoparticle (LNP) composition and a second LNP composition, wherein the first LNP composition comprises a first guide RNA (gRNA) directed to a first target sequence and a first nucleic acid genome editing tool and the second LNP composition comprises a second gRNA directed to a second target sequence different from the first target sequence and a second nucleic acid genome editing tool; and
b. expanding the cell in vitro;
thereby producing multiple genome edits in the cell.

27-28. (canceled)

29. The method of claim 26, wherein the first nucleic acid genome editing tool or the second nucleic acid genome editing tool comprises a nucleic acid encoding an RNA-guided DNA binding agent.

30. The method of claim 26, wherein the cell is further contacted with a donor nucleic acid for insertion in a target sequence.

31-32. (canceled)

33. A method of delivering lipid nanoparticle (LNP) compositions to a population of in vitro-cultured cells, comprising the steps of:
a. contacting the population of cells in vitro with at least a first LNP composition comprising a first nucleic acid, thereby producing a contacted population of cells;
b. culturing the contacted population of cells in vitro, thereby producing a population of cultured contacted cells;
c. contacting the contacted population of cells or the population of cultured contacted cells in vitro with at least a second LNP composition comprising a second nucleic acid, wherein the second nucleic acid is different from the first nucleic acid; and
(i) wherein at least 70%, 80%, 90%, or 95% of the cells in the population of cells are viable 24 hours after the last contact with an LNP composition, or
(ii) wherein the method further comprises: d. expanding the population of cells in vitro, and wherein the expanded population of cells exhibits a survival rate of at least 70%, 80%, 90%, or 95% at 24 hours of expansion.

34-39. (canceled)

40. A method of gene editing in a population of cells, comprising the steps of:
a. contacting the population of cells in vitro with a first lipid nanoparticle (LNP) composition comprising a first genome editing tool and a second LNP composition comprising a second genome editing tool; and
b-1. expanding the population of cells in vitro, or
b-2. culturing the population of cells in vitro, wherein at least 70%, 80%, 90%, or 95% of the cells in the population of cells are viable 24 hours after the last contact with an LNP composition;
thereby editing the population of cells.

41. (canceled)

42. The method of claim 40, wherein the first genome editing tool or the first genome editing tool comprises a guide RNA.

43. (canceled)

44. The method of claim 40, wherein at least one of the LNP compositions comprises an RNA-guided DNA binding agent.

45-49. (canceled)

50. The method of claim 26, wherein the cell is a T cell.

51-54. (canceled)

55. A method of producing multiple genome edits in an in vitro-cultured T cell, comprising the steps of:
a. contacting the T cell in vitro with (i) a first lipid nanoparticle (LNP) composition comprising a guide RNA (gRNA) directed to a first target sequence and (ii) one or two additional LNP compositions, wherein each additional LNP composition comprises a gRNA directed to a target sequence that differs from the first target sequence or a genome editing tool;
b. activating the T cell in vitro;
c. contacting the activated T cell in vitro with (i) a further LNP composition comprising a further guide RNA directed to a target sequence that differs from the target sequence(s) of (a) and (ii) one or more LNP compositions, wherein each LNP composition comprises a guide RNA directed to a target sequence that differs from the target sequence(s) of (a) and from each other or a genome editing tool;
d. expanding the cell in vitro;
thereby producing multiple genome edits in the T cell.

56-85. (canceled)

86. The method of claim 26, wherein the first nucleic acid genome editing tool or the second nucleic acid genome editing tool comprises a donor nucleic acid.

87. The method of claim 26, wherein the first nucleic acid genome editing tool or the second nucleic acid genome editing tool comprises an RNA-guided DNA binding agent.

88-109. (canceled)

110. The method of claim 26, wherein the method further comprises contacting the cell with one or more donor nucleic acids, wherein at least one of the one or more donor nucleic acids comprises regions having homology with corresponding regions of a TRAC locus, a B2M locus, an AAVS1 locus, a CIITA locus, or a TRBC locus.

111-128. (canceled)

129. The method of claim 40, wherein the cells are T cells, and wherein at least 95% of the cells in the edited population comprise a genome edit of an endogenous T cell receptor (TCR) sequence.

130-145. (canceled)

146. The method of claim 40, wherein the first LNP composition or the second LNP composition comprises an amine lipid, a helper lipid, and a PEG lipid.

147-169. (canceled)

170. A cell population made by or obtainable by the method of claim 40.

171. (canceled)

172. A method of treating cancer or an autoimmune disease, comprising administering an effective amount of the population of claim 40.

173-175. (canceled)


SEQUENCE LISTING (723 sequences)

SEQ ID NO: 1 - DNA, 4140 bp, synthetic construct
  misc_feature 1..4140: note = Synthetic: ORF encoding Sp. Cas9
  source 1..4140: mol_type = other DNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 2 - DNA, 4140 bp, synthetic construct
  misc_feature 1..4140: note = Synthetic: ORF encoding Sp. Cas9
  source 1..4140: mol_type = other DNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 3 - RNA, 4197 nt, synthetic construct
  misc_feature 1..4197: note = Synthetic: open reading frame for Cas9 with Hibit tag
  source 1..4197: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctga

SEQ ID NO: 4-5 - skipped (2 entries)

SEQ ID NO: 6 - protein, 1379 aa, synthetic construct
  REGION 1..1379: note = Synthetic: amino acid sequence encoded by SEQ ID NOs: 1-3 of Cas9
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 7 - protein, 1398 aa, synthetic construct
  REGION 1..1398: note = Synthetic: amino acid sequence for Sp Cas9-Hibit fusion
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKVSESATPESVSGWRLFKKIS

SEQ ID NO: 8 - DNA, 720 bp, synthetic construct
  misc_feature 1..720: note = Synthetic: GFP insert for HDRT GFP: P00894
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 9 - DNA, 4305 bp, synthetic construct
  misc_feature 1..4305: note = Synthetic: Full HDRT template - transgenic WT1 TCR and TRAC homology arms
  source 1..4305: mol_type = other DNA
tgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgcggccgccaccatgggatcttggacactgtgttgcgtgtccctgtgcatcctggtggccaagcacacagatgccggcgtgatccagtctcctagacacgaagtgaccgagatgggccaagaagtgaccctgcgctgcaagcctatcagcggccacgattacctgttctggtacagacagaccatgatgagaggcctggaactgctgatctacttcaacaacaacgtgcccatcgacgacagcggcatgcccgaggatagattcagcgccaagatgcccaacgccagcttcagcaccctgaagatccagcctagcgagcccagagatagcgccgtgtacttctgcgccagcagaaagacaggcggctacagcaatcagccccagcactttggagatggcacccggctgagcatcctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggcggctccggtgccacaaacttctccctgctcaagcaggccggagatgtggaagagaaccctggccctatggaaaccctgctgaaggtgctgagcggcacactgctgtggcagctgacatgggtccgatctcagcagcctgtgcagtctcctcaggccgtgattctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgtggcaccgcctggatcaacgactacaagctgtctttcggagccggcaccacagtgacagtgcgggccaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagactgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttcagaatcctgctgctcaaggtggccggcttcaacctgctgatgaccctgagactgtggtccagctaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctct

SEQ ID NO: 10 - protein, 314 aa, synthetic construct
  REGION 1..314: note = Synthetic: TCR beta chain pINT1066
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRKTGGYSNQPQHFGDGTRLSILEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 11 - protein, 272 aa, synthetic construct
  REGION 1..272: note = Synthetic: TCR alpha chain pINT1066
METLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTAWINDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 12 - protein, 608 aa, synthetic construct
  REGION 1..608: note = Synthetic: TCR beta-linker-alpha configuration pINT1066
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRKTGGYSNQPQHFGDGTRLSILEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMETLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTAWINDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 13 - DNA, 1556 bp, synthetic construct
  misc_feature 1..1556: note = Synthetic: Full HDRT template - GFP T2A insert GFP: P00894
  source 1..1556: mol_type = other DNA
gagggccgcggcagcctgctgacctgcggcgacgtggaggagaatcccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctct

SEQ ID NO: 14 - DNA, 720 bp, synthetic construct; identical to SEQ ID NO: 8
  misc_feature 1..720: note = Synthetic: eGFP ORF GFP: P00894, GFP P01018
  source 1..720: mol_type = other DNA

SEQ ID NO: 15 - DNA, 3105 bp, synthetic construct
  misc_feature 1..3105: note = Synthetic: Full HDRT template - GFP with B2M homology arms GFP: P01018
  source 1..3105: mol_type = other DNA
agatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacggccggccccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagttgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggct

SEQ ID NO: 16 - protein, 1379 aa, synthetic construct; identical to SEQ ID NO: 6
  REGION 1..1379: note = Synthetic: Cas9 amino acid sequence for RNP

SEQ ID NO: 17 - RNA, 5399 nt, synthetic construct
  misc_feature 1..5399: note = Synthetic: mRNA encoding BC22n with Hibit tag
  source 1..5399: mol_type = other RNA
gggaagctcagaataaacgctcaactttggccggatctgccaccatggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaaagttaaaaaaaaaaaactgaaaaaaaaaaaatttaaaaaaaaaaaatctag

SEQ ID NO: 18 - RNA, 4839 nt, synthetic construct
  misc_feature 1..4839: note = Synthetic: Open reading frame for BC22n with Hibit tag
  source 1..4839: mol_type = other RNA
atggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctga

SEQ ID NO: 19 - protein, 1612 aa, synthetic construct
  REGION 1..1612: note = Synthetic: Amino acid sequence for BC22n with Hibit tag
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKVSESATPESVSGWRLFKKIS

SEQ ID NO: 20 - RNA, 589 nt, synthetic construct
  misc_feature 1..589: note = Synthetic: mRNA encoding UGI
  source 1..589: mol_type = other RNA
gggagacccaagctggctagctcccgcagtcggcgtccagcggctctgcttgttcgtgtgtgtgtcgttgcaggccttattcggatccgccaccatgggaccgaagaagaagagaaaggtcggaggaggaagcacaaacctgtcggacatcatcgaaaaggaaacaggaaagcagctggtcatccaggaatcgatcctgatgctgccggaagaagtcgaagaagtcatcggaaacaagccggaatcggacatcctggtccacacagcatacgacgaatcgacagacgaaaacgtcatgctgctgacatcggacgcaccggaatacaagccgtgggcactggtcatccaggactcgaacggagaaaacaagatcaagatgctgtgatagtctagacatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagtctag

SEQ ID NO: 21 - RNA, 291 nt, synthetic construct
  misc_feature 1..291: note = Synthetic: Open reading frame for UGI
  source 1..291: mol_type = other RNA
atgggaccgaagaagaagagaaaggtcggaggaggaagcacaaacctgtcggacatcatcgaaaaggaaacaggaaagcagctggtcatccaggaatcgatcctgatgctgccggaagaagtcgaagaagtcatcggaaacaagccggaatcggacatcctggtccacacagcatacgacgaatcgacagacgaaaacgtcatgctgctgacatcggacgcaccggaatacaagccgtgggcactggtcatccaggactcgaacggagaaaacaagatcaagatgctgtga

SEQ ID NO: 22 - protein, 107 aa, synthetic construct
  REGION 1..107: note = Synthetic: Amino acid sequence for UGI
MTNLSDIIEKETGKQLVIQESILMLPEEVEEVIGNKPESDILVHTAYDESTDENVMLLTSDAPEYKPWALVIQDSNGENKIKMLSGGSKRTADGSEFESPKKKRKVE

SEQ ID NO: 23 - protein, 7 aa, synthetic construct
  REGION 1..7: note = Synthetic: SV40 NLS
PKKKRKV

SEQ ID NO: 24 - protein, 7 aa, synthetic construct
  REGION 1..7: note = Synthetic: SV40 NLS
PKKKRRV

SEQ ID NO: 25 - protein, 16 aa, synthetic construct
  REGION 1..16: note = Synthetic: NLS
KRPAATKKAGQAKKKK

SEQ ID NO: 26 - RNA, 10 nt, synthetic construct
  misc_feature 1..10: note = Synthetic: Kozak sequence
  source 1..10: mol_type = other RNA
gccrccatgg

SEQ ID NO: 27 - RNA, 13 nt, synthetic construct
  misc_feature 1..13: note = Synthetic: Kozak sequence
  source 1..13: mol_type = other RNA
gccgccrccatgg

SEQ ID NO: 28 - RNA, 100 nt, synthetic construct
  misc_feature 1..100: note = Synthetic: sgRNA sequence
  modified_base 1..3: mod_base = OTHER; note = 2'-O-Me and PS bond
  variation 1..20: note = n is any natural or non-natural nucleotide
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 29-700 - skipped (672 entries)

SEQ ID NO: 701 - RNA, 100 nt, synthetic construct
  misc_feature 1..100: note = Synthetic: gRNA G000529
  modified_base 1..3: mod_base = OTHER; note = 2'-O-Me and PS bond
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA
ggccacggagcgagacatctgttttagagctagaaatagcaagttaaaataaggctagtccgttatcaacttgaaaaagtggcaccgagtcggtgctttt

SEQ ID NO: 702 - RNA, 100 nt, synthetic construct
  misc_feature 1..100: note = Synthetic: gRNA G013674
  modified_base 1..3: mod_base = OTHER; note = 2'-O-Me and PS bond
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA
ttctaggggccccaactccagttttagagctagaaatagcaagttaaaataaggctagtccgttatcaacttgaaaaagtggcaccgagtcggtgctttt

SEQ ID NO: 703 - RNA, 100 nt, synthetic construct
  misc_feature 1..100: note = Synthetic: gRNA G012086
  modified_base 1..3: mod_base = OTHER; note = 2'-O-Me and PS bond
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA
agagtctctcagctggtacagttttagagctagaaatagcaagttaaaataaggctagtccgttatcaacttgaaaaagtggcaccgagtcggtgctttt

SEQ ID NO: 704-706 - skipped (3 entries)

SEQ ID NO: 707 - RNA, 100 nt, synthetic construct
  misc_feature 1..100: note = Synthetic: gRNA G016239
  modified_base 1..3: mod_base = OTHER; note = 2'-O-Me and PS bond
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA
ggcctcggcgctgacgatctgttttagagctagaaatagcaagttaaaataaggctagtccgttatcaacttgaaaaagtggcaccgagtcggtgctttt

SEQ ID NO: 708 - RNA, 100 nt, synthetic construct
  misc_feature 1..100: note = Synthetic: gRNA G013006
  modified_base 1..3: mod_base = OTHER; note = 2'-O-Me and PS bond
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA
ctctcagctggtacacggcagttttagagctagaaatagcaagttaaaataaggctagtccgttatcaacttgaaaaagtggcaccgagtcggtgctttt

SEQ ID NO: 709 - RNA, 100 nt, synthetic construct; identical to SEQ ID NO: 701
  misc_feature 1..100: note = Synthetic: gRNA G012738
  modified_base 1..3: mod_base = OTHER; note = 2'-O-Me and PS bond
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA

SEQ ID NO: 710 - RNA, 100 nt, synthetic construct
  misc_feature 1..100: note = Synthetic: gRNA G000562
  modified_base 1..3: mod_base = OTHER; note = 2'-O-Me and PS bond
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA
ccaatatcaggagactaggagttttagagctagaaatagcaagttaaaataaggctagtccgttatcaacttgaaaaagtggcaccgagtcggtgctttt

SEQ ID NO: 711 - RNA, 100 nt, synthetic construct
  misc_feature 1..100: note = Synthetic: gRNA G015995
  modified_base 1..3: mod_base = OTHER; note = 2'-O-Me and PS bond
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA
ttaccccacttaactatcttgttttagagctagaaatagcaagttaaaataaggctagtccgttatcaacttgaaaaagtggcaccgagtcggtgctttt

SEQ ID NO: 712 - RNA, 100 nt, synthetic construct
  misc_feature 1..100: note = Synthetic: gRNA G016017
  modified_base 1..3: mod_base = OTHER; note = 2'-O-Me and PS bond
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA
ccactctgccccatgggctcgttttagagctagaaatagcaagttaaaataaggctagtccgttatcaacttgaaaaagtggcaccgagtcggtgctttt

SEQ ID NO: 713 - RNA, 100 nt, synthetic construct
  misc_feature 1..100: note = Synthetic: gRNA G016206
  modified_base 1..3: mod_base = OTHER; note = 2'-O-Me and PS bond
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA
cgctgtcaagtccagttctagttttagagctagaaatagcaagttaaaataaggctagtccgttatcaacttgaaaaagtggcaccgagtcggtgctttt

SEQ ID NO: 714 - RNA, 100 nt, synthetic construct
  misc_feature 1..100: note = Synthetic: gRNA G018117
  modified_base 1..3: mod_base = OTHER; note = 2'-O-Me and PS bond
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA
gcgtccacatcctgcaaggggttttagagctagaaatagcaagttaaaataaggctagtccgttatcaacttgaaaaagtggcaccgagtcggtgctttt

SEQ ID NO: 715 - RNA, 100 nt, synthetic construct
  misc_feature 1..100: note = Synthetic: gRNA G013676
  modified_base 1..3: mod_base = OTHER; note = 2'-O-Me and PS bond
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA
tggtcagggcaagagctattgttttagagctagaaatagcaagttaaaataaggctagtccgttatcaacttgaaaaagtggcaccgagtcggtgctttt

SEQ ID NO: 716 - RNA, 100 nt, synthetic construct
  misc_feature 1..100: note = Synthetic: gRNA G018995
  modified_base 1..3: mod_base = OTHER; note = 2'-O-Me and PS bond
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA
acagcgacgccgcgagccaggttttagagctagaaatagcaagttaaaataaggctagtccgttatcaacttgaaaaagtggcaccgagtcggtgctttt

SEQ ID NO: 717 - DNA, 4607 bp, synthetic construct
  misc_feature 1..4607: note = Synthetic: pINT1405, HD1 TCR insertion including ITRs
  source 1..4607: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgcggccgccaccatgggatcttggacactgtgttgcgtgtccctgtgcatcctggtggccaagcacacagatgccggcgtgatccagtctcctagacacgaagtgaccgagatgggccaagaagtgaccctgcgctgcaagcctatcagcggccacgattacctgttctggtacagacagaccatgatgagaggcctggaactgctgatctacttcaacaacaacgtgcccatcgacgacagcggcatgcccgaggatagattcagcgccaagatgcccaacgccagcttcagcaccctgaagatccagcctagcgagcccagagatagcgccgtgtacttctgcgccagcagaaagacaggcggctacagcaatcagccccagcactttggagatggcacccggctgagcatcctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggcggctccggtgccacaaacttctccctgctcaagcaggccggagatgtggaagagaaccctggccctatggaaaccctgctgaaggtgctgagcggcacactgctgtggcagctgacatgggtccgatctcagcagcctgtgcagtctcctcaggccgtgattctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgtggcaccgcctggatcaacgactacaagctgtctttcggagccggcaccacagtgacagtgcgggccaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagactgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttcagaatcctgctgctcaaggtggccggcttcaacctgctgatgaccctgagactgtggtccagctaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 718 - RNA, 100 nt, synthetic construct
  misc_feature 1..100: note = Synthetic: gRNA G016200
  modified_base 1..3: mod_base = OTHER; note = 2'-O-Me and PS bond
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA
ccacacccaaaaggccacacgttttagagctagaaatagcaagttaaaataaggctagtccgttatcaacttgaaaaagtggcaccgagtcggtgctttt

SEQ ID NO: 719 - RNA, 100 nt, synthetic construct
  misc_feature 1..100: note = Synthetic: gRNA G016086
  modified_base 1..3: mod_base = OTHER; note = 2'-O-Me and PS bond
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA
cgcccaggtcctcacgtctggttttagagctagaaatagcaagttaaaataaggctagtccgttatcaacttgaaaaagtggcaccgagtcggtgctttt

SEQ ID NO: 720 - DNA, 3294 bp, synthetic construct
  misc_feature 1..3294: note = Synthetic: AAV6-1008 GFP insert for AAVS1
  source 1..3294: mol_type = other DNA
tgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttccgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaacatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgctagcgctaccggactcaatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgttagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgt

SEQ ID NO: 721 - DNA, 4250 bp, synthetic construct
  misc_feature 1..4250: note = Synthetic: AAV6-231 GFP insert for AAVS1
  source 1..4250: mol_type = other DNA
gaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttagttattaatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaacatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgctagcgctaccggactcaatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtgtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctccaa

SEQ ID NO: 722 - DNA, 3105 bp, synthetic construct; identical to SEQ ID NO: 15
  misc_feature 1..3105: note = Synthetic: AAV6-1018 GFP insert for B2M
  source 1..3105: mol_type = other DNA

SEQ ID NO: 723 - RNA, 100 nt, synthetic construct
  misc_feature 1..100: note = Synthetic: G014832
  modified_base 1..3: mod_base = OTHER; note = 2'-O-Me and PS bond
  modified_base 29..40: mod_base = OTHER; note = 2'-O-Me
  modified_base 69..100: mod_base = OTHER; note = 2'-O-Me
  modified_base 97..99: mod_base = OTHER; note = PS bond
  source 1..100: mol_type = other RNA
ggctctcggagaatgacgaggttttagagctagaaatagcaagttaaaataaggctagtccgttatcaacttgaaaaagtggcaccgagtcggtgctttt